(19) United States
(12) Patent Application Publication
(10) Pub. No.: US 2025/0000973 A1
(43) Pub. Date: Jan. 2, 2025

(54) METHODS OF MAKING CHIMERIC ANTIGEN RECEPTOR-EXPRESSING CELLS

(71) Applicant: Novartis AG, Basel (CH)

(72) Inventors: Michael Bardroff, Loerrach (DE); Regis Cebe, Basel (CH); Brian Walter Granda, Salisbury, MA (US); Shyamali Jayashankar, Walnut Creek, CA (US); Sandeep Tharian Koshy, Cambridge, MA (US); Sandra Miller, Munich (DE); Andrew Patrick Price, Cambridge, MA (US); Amy Rayo, Cambridge, MA (US); Darko Skegro, Riehen (CH); Louise Mary Treanor, Cambridge, MA (US); Jennifer Yang, Somerville, MA (US)

(21) Appl. No.: 18/684,417

(22) PCT Filed: Aug. 19, 2022
(86) PCT No.: PCT/IB2022/057799

§ 371 Date: Feb. 16, 2024

Related U.S. Application Data
(60) Provisional application No. 63/235,634, filed on Aug. 20, 2021.

(51) Int. Cl. A61K 39/00; A61P 35/00; C07K 14/705; C07K 14/725; C07K 16/28; C12N 5/0783

(52) U.S. Cl. CPC A61K 39/4611; A61K 39/4631; A61K 39/464402; A61K 39/464412; A61P 35/00; C07K 14/7051; C07K 14/70517; C07K 14/70578; C07K 16/2803; C07K 16/2809; C07K 16/2818; C07K 16/2878; C12N 5/0636; C07K 2317/53; C07K 2317/622; C07K 2319/02; C07K 2319/03; C12N 2501/2302; C12N 2501/2306; C12N 2501/2307; C12N 2501/2315

(57) ABSTRACT

The invention provides methods of making immune effector cells (for example, T cells, NK cells) that express a chimeric antigen receptor (CAR), and compositions generated by such methods.

### RELATED APPLICATIONS

This application claims priority to U.S. Provisional Application 63/235,634 filed on Aug. 20, 2021, the entire contents of which are hereby incorporated by reference.

### FIELD OF THE INVENTION

The present invention relates generally to methods of making immune effector cells (for example, T cells or NK cells) engineered to express a Chimeric Antigen Receptor (CAR), and compositions comprising the same.

### SEQUENCE LISTING

The instant application contains a Sequence Listing which has been submitted electronically in XML format compliant with WIPO Standard ST.26 and is hereby incorporated by reference in its entirety. Said XLM copy, created on Aug. 15, 2022, is named N2067-7191WO.XML and is 962.9 kb in size.

### BACKGROUND OF THE INVENTION

Adoptive cell transfer (ACT) therapy with T cells, especially with T cells transduced with Chimeric Antigen Receptors (CARs), has shown promise in several hematologic cancer trials. The manufacture of gene-modified T cells is currently a complex process. There exists a need for methods and processes to improve production of the CAR-expressing cell therapy product, enhance product quality, and maximize the therapeutic efficacy of the product.

### SUMMARY OF THE INVENTION

The present disclosure pertains to methods of making immune effector cells (for example, T cells or NK cells) engineered to express a CAR, and compositions generated using such methods. Also disclosed are methods of using such compositions for treating a disease, for example, cancer, in a subject.

In some aspects, the disclosure provides a method of making a population of cells (for example, T cells) that express a chimeric antigen receptor (CAR). The method comprises: (i) contacting (for example, binding) a population of cells (for example, T cells, for example, T cells isolated from a frozen or fresh leukapheresis product) with a multispecific binding molecule comprising (A) an anti-CD3 binding domain, and (B) a costimulatory molecule binding domain (e.g., an anti-CD2 binding domain or an anti-CD28 binding domain), and (C) an Fc region comprising: a L234A, L235A, S267K, and P329A mutation (LALASKPA), numbered according to the EU numbering system; a L234A, L235A, and P329G mutation (LALAPG), numbered according to the EU numbering system; a G237A, D265A, P329A, and S267K mutation (GADAPASK), numbered according to the EU numbering system; a L234A, L235A, and G237A mutation (LALGA), numbered according to the EU numbering system; a D265A, P329A, and S267K mutation (DAPASK), numbered according to the EU numbering system; a G237A, D265A, and P329A mutation (GADAPA), numbered according to the EU numbering system; or a L234A, L235A, and P329A mutation (LALAPA), numbered according to the EU numbering system; (ii) contacting the population of cells (for example, T cells) with a nucleic acid molecule (for example, a DNA or RNA molecule) encoding the CAR, thereby providing a population of cells (for example, T cells) comprising the nucleic acid molecule, and (iii) harvesting the population of cells (for example, T cells) for storage (for example, reformulating the population of cells in cryopreservation media) or administration, wherein: (a) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 26 hours after the beginning of step (i), for example, no later than 22, 23, 24, or 25 hours after the beginning of step (i), for example, no later than 24 hours after the beginning of step (i); (b) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 30, 36, or 48 hours after the beginning of step (ii), for 25 example, no later than 22, 23, 24, 25, 26, 27, 28, 29, 30, 31, 32, 33, 34, 35, 36, 37, 38, 39, 40, 41, 42, 43, 44, 45, 46, 47, 48 hours after the beginning of step (ii); or (c) the population of cells from step (iii) are not expanded, or expanded by no more than 5, 6, 7, 8, 9, 10, 11, 12, 13, 14, 15, 16, 17, 18, 19, 20, 25, 30, 35, or 40%, for example, no more than 10%, for example, as assessed by the number of living cells, compared to the population of cells at the beginning of step (i). In some embodiments, the nucleic acid molecule in step (ii) is a DNA molecule. In some embodiments, the nucleic acid molecule in step (ii) is an RNA molecule. In some embodiments, the nucleic acid molecule in step (ii) is on a viral vector, for example, a viral vector chosen from a lentivirus vector, an adenoviral vector, or a retrovirus vector. In some embodiments, the nucleic acid molecule in step (ii) is on a non-viral vector. In some embodiments, the nucleic acid molecule in step (ii) is on a plasmid. In some embodiments, the nucleic acid molecule in step (ii) is not on any vector. In some embodiments, step (ii) comprises transducing the population of cells (for example, T cells) with a viral vector comprising a nucleic acid molecule encoding the CAR.

In some aspects, the disclosure provides a multispecific binding molecule comprising (A) an anti-CD3 binding domain, (B) a costimulatory molecule binding domain (e.g., an anti-CD2 binding domain or an anti-CD28 binding domain); and optionally (C) an Fc region comprising: a L234A, L235A, S267K, and P329A mutation (LALASKPA), numbered according to the EU numbering system; a L234A, L235A, and P329G mutation (LALAPG), numbered according to the EU numbering system; a G237A, D265A, P329A, and S267K mutation (GADAPASK), numbered according to the EU numbering system; a L234A, L235A, and G237A mutation (LALGA), numbered according to the EU numbering system; a D265A, P329A, and S267K mutation (DAPASK), numbered according to the EU numbering system; a G237A, D265A, and P329A mutation (GADAPA), numbered according to the EU numbering system; or a L234A, L235A, and P329A mutation (LALAPA), numbered according to the EU numbering system.

In some embodiments, the anti-CD3 binding domain, e.g., an anti-CD3 scFv, is situated N-terminal of the costimulatory molecule binding domain, e.g., an anti-CD2 Fab or an anti-CD28 Fab. In some embodiments, the anti-CD3 binding domain, e.g., an anti-CD3 scFv, is situated C-terminal of the costimulatory molecule binding domain, e.g., an anti-CD2 Fab or an anti-CD28 Fab. In some embodiments, the Fc region comprises a CH2. In some embodiments, the Fc region comprises a CH3. In some embodiments, the Fc region is situated between the anti-CD3 binding domain and the costimulatory molecule binding domain. In some embodiments, the anti-CD3 binding domain is situated C-terminal of the CH2. In some embodiments, the anti-CD3 binding domain is situated N-terminal of the CH2.

In some embodiments of the compositions and methods herein, the multispecific binding molecule comprises: (i) a first polypeptide comprising from N-terminal to C-terminal: VH of the anti-CD3 binding domain, VL of the anti-CD3 binding domain, VH of the costimulatory molecule binding domain, CH1, CH2, and CH3; and (ii) a second polypeptide comprising from N-terminal to C-terminal: VL of the costimulatory molecule binding domain and CL. In some embodiments, the multispecific binding molecule comprises: (i) a first polypeptide comprising from N-terminal to C-terminal: VH of the costimulatory molecule binding domain, CH1, CH2, CH3, VH of the anti-CD3 binding domain, and VL of the anti-CD3 binding domain; and (ii) a second polypeptide comprising from N-terminal to C-terminal: VL of the costimulatory molecule binding domain and CL. In some embodiments, the multispecific binding molecule comprises: (i) a first polypeptide comprising from N-terminal to C-terminal: VH of the costimulatory molecule binding domain, CH1, VH of the anti-CD3 binding domain, VL of the anti-CD3 binding domain, CH2, and CH3; and (ii) a second polypeptide comprising from N-terminal to C-terminal: VL of the costimulatory molecule binding domain and CL. In some embodiments, the anti-CD3 binding domain comprises an scFv and the costimulatory molecule binding domain is part of a Fab fragment.

In some embodiments, the anti-CD3 binding domain comprises: (i) a variable heavy chain region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3 of an anti-CD3 antibody molecule of Table 27 (for example the anti-CD3 (1), anti-CD3 (2), anti-CD3 (3), or anti-CD3 (4)); and/or (ii) the amino acid sequence of any one of the VH and/or VL region of an 20) anti-CD3 antibody molecule provided in Table 27 (for example the anti-CD3 (1), anti-CD3 (2), anti-CD3 (3), or anti-CD3 (4)), or an amino acid sequence at least 95% identical thereto.

In some embodiments, the costimulatory molecule binding domain is an anti-CD2 antigen binding domain. In some embodiments, the anti-CD2 antigen binding domain comprises: (i) a VH comprising a HCDR1, a HCDR2, and a HCDR3, and a VL comprising a LCDR1, a LCDR2, and a LCDR3 of an anti-CD2 antibody molecule of Table 27 (for example the anti-CD2 (1)); and/or (ii) the amino acid sequence of any one of the VH and/or VL region of an anti-CD2 antibody molecule provided in Table 27 (for example the anti-CD2 (1)), or an amino acid sequence at least 95% identical thereto.

In some embodiments, the costimulatory molecule binding domain is an anti-CD28 antigen binding domain. In some embodiments, the anti-CD28 antigen binding domain comprises: (i) a VH comprising a HCDR1, a HCDR2, and a HCDR3, and a VL comprising a LCDR1, a LCDR2, and a LCDR3 of an anti-CD28 antibody molecule of Table 27 (for example the anti-CD28 (1) or anti-CD28 (2)); and/or (ii) the amino acid sequence of any one of the VH and/or VL region of an anti-CD28 antibody molecule provided in Table 27 (for example the anti-CD28 (1) or anti-CD28 (2)), or an amino acid sequence at least 95% identical thereto.

In some embodiments, the anti-CD3 binding domain comprises an scFv. In some embodiments, the anti-CD3 binding domain comprises a VH linked to a VL by a peptide linker, e.g., a glycine-serine linker, e.g., a (G₄S)₄ linker. In some embodiments, the anti-CD3 binding domain comprises a VH and a VL, wherein the VH is N-terminal of the VL.

In some embodiments, the costimulatory molecule binding domain is part of a Fab fragment, e.g., a Fab fragment that is part of a polypeptide sequence that comprises an Fc domain, optionally wherein the Fc domain comprises an amino acid sequence provided in Table 28, or a sequence with at least 95% sequence identity thereto.

In some embodiments, the anti-CD3 binding domain is situated N-terminal of the costimulatory molecule binding domain. In some embodiments, the anti-CD3 binding domain is linked to the costimulatory molecule binding domain by a peptide linker, e.g., a glycine-serine linker, e.g., a (G4S) 4 linker.

In some embodiments, the anti-CD3 binding domain is situated C-terminal of the costimulatory molecule binding domain, wherein optionally an Fc region is situated between the anti-CD3 binding domain and the costimulatory molecule binding domain.

In some embodiments, the multispecific binding molecule comprises a CH1. In some embodiments, the CH1 is C-terminal of the VH of the costimulatory molecule binding domain.

In some embodiments, the multispecific binding molecule comprises one or both of a CH2 and a CH3.

In some embodiments, the anti-CD3 binding domain is linked to the CH3 by a peptide linker, e.g., a glycine-serine linker, e.g., a (G4S) 4 linker. In some embodiments, the anti-CD3 binding domain is situated C-terminal of the CH1. In some embodiments, the construct comprises a CH2, and the anti-CD3 binding domain is situated N-terminal of the CH2. In some embodiments, anti-CD3 binding domain is linked to the CH1 by a peptide linker, e.g., a glycine-serine linker, e.g., a (G4S) 2 linker. In some embodiments, the anti-CD3 binding domain is linked to the CH2 by a peptide linker, e.g., a glycine-serine linker, e.g., a (G4S) 4 linker.

In some embodiments, the multispecific binding molecule further comprises a CL. In some embodiments, the CL is C-terminal of the VL of the costimulatory molecule binding domain. In some embodiments, the CL domain is linked to the CH1, e.g., via a disulfide bridge.

In some embodiments, the multispecific binding molecule comprises: (i) the amino acid sequence of any heavy chain provided in Table 28, or an amino acid sequence with at least 95% sequence identity thereto; and/or (ii) the amino acid sequence of any light chain provided in Table 28, or an amino acid sequence with at least 95% sequence identity thereto.

In some embodiments, this invention features a method of making a population of cells (for example, T cells) that express a chimeric antigen receptor (CAR), the method comprising: (i) contacting (for example, binding) a population of cells (for example, T cells, for example, T cells isolated from a frozen or fresh leukapheresis product) with (A) an agent that stimulates a CD3/TCR complex and/or (B) an agent that stimulates a costimulatory molecule and/or growth factor receptor on the surface of the cells; (ii) contacting the population of cells (for example, T cells) with a nucleic acid molecule (for example, a DNA or RNA molecule) encoding the CAR, thereby providing a population of cells (for example, T cells) comprising the nucleic acid molecule, and (iii) harvesting the population of cells (for example, T cells) for storage (for example, reformulating the population of cells in cryopreservation media) or administration, wherein: (a) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 26 hours after the beginning of step (i), for example, no later than 22, 23, 24, or 25 hours after the beginning of step (i), for example, no later than 24 hours after the beginning of step (i); (b) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 30, 36, or 48 hours after the beginning of step (ii), for example, no later than 22, 23, 24, 25, 26, 27, 28, 29, 30, 31, 32, 33, 34, 35, 36, 37, 38, 39, 40, 41, 42, 43, 44, 45, 46, 47, 48 hours after the beginning of step (ii); or (c) the population of cells from step (iii) are not expanded, or expanded by no more than 5, 6, 7, 8, 9, 10, 11, 12, 13, 14, 15, 16, 17, 18, 19, 20, 25, 30, 35, or 40%, for example, no more than 10%, for example, as assessed by the number of living cells, compared to the population of cells at the beginning of step (i). In some embodiments, the nucleic acid molecule in step (ii) is a DNA molecule. In some embodiments, the nucleic acid molecule in step (ii) is an RNA molecule. In some embodiments, the nucleic acid molecule in step (ii) is on a viral vector, for example, a viral vector chosen from a lentivirus vector, an adenoviral vector, or a retrovirus vector. In some embodiments, the nucleic acid molecule in step (ii) is on a non-viral vector. In some embodiments, the nucleic acid molecule in step (ii) is on a plasmid. In some embodiments, the nucleic acid molecule in step (ii) is not on any vector. In some embodiments, step (ii) comprises transducing the population of cells (for example, T cells) with a viral vector comprising a nucleic acid molecule encoding the CAR. In some embodiments, step (ii) further comprises contacting the population of cells (for example, T cells) with shRNA that targets Tet2 comprising (A) a sense strand comprising a Tet2 target sequence and (B) an antisense strand complementary to the sense strand in whole or in part or a vector encoding the shRNA. In some embodiments, sense strand comprises the Tet2 target sequence GGGTAAGCCAAGAAAGAAA (SEQ ID NO: 418). In some embodiments, the anti-sense strand comprises the reverse complement thereof, i.e. TTTCTTTCTTGGCTTACCC (SEQ ID NO: 419). In some embodiments, the vector encoding the shRNA is the same or different from the vector encoding the CAR. In some embodiments, the vector encoding the shRNA sequence comprises promoter (such as but not limited to a U6 promoter), a sense strand comprising a Tet2 target sequence, a loop, an anti-sense strand complementary to the sense strand in whole or in part, and, optionally, a polyT tail, e.g. the sequences in Table 29. In some embodiments, step (ii) is performed together with step (i). In some embodiments, step (ii) is performed no later than 20 hours after the beginning of step (i). In some embodiments, step (ii) is performed no later than 12, 13, 14, 15, 16, 17, or 18 hours after the beginning of step (i). In some embodiments, step (ii) is performed no later than 18 hours after the beginning of step (i). In some embodiments, step (iii) is performed no later than 26 hours after the beginning of step (i). In some embodiments, step (iii) is performed no later than 22, 23, 24, or 25 hours after the beginning of step (i). In some embodiments, step (iii) is performed no later than 24 hours after the beginning of step (i). In some embodiments, step (iii) is performed no later than 30, 36, or 48 hours after the beginning of step (ii). In some embodiments, step (iii) is performed no later than 22, 23, 24, 25, 26, 27, 28, 29, 30, 31, 32, 33, 34, 35, 36, 37, 38, 39, 40, 41, 42, 43, 44, 45, 46, 47, 48 hours after the beginning of step (ii).

In some embodiments, the population of cells from step (iii) are not expanded. In some embodiments, the population of cells from step (iii) are expanded by no more than 5, 6, 7, 8, 9, 10, 11, 12, 13, 14, 15, 16, 17, 18, 19, 20, 25, 30, 35, or 40%, for example, as assessed by the number of living cells, compared to the population of cells at the beginning of step (i). In some embodiments, the population of cells from step (iii) are expanded by no more than 10%, for example, as assessed by the number of living cells, compared to the population of cells at the beginning of step (i).

In some embodiments, the agent that stimulates a CD3/TCR complex is an agent that stimulates CD3. In some embodiments, the agent that stimulates a costimulatory molecule and/or growth factor receptor is an agent that stimulates CD28, ICOS, CD27, HVEM, LIGHT, CD40, 4-1BB, OX40, DR3, GITR, CD30, TIM1, CD2, CD226, or any combination thereof. In some embodiments, the agent that stimulates a costimulatory molecule and/or growth factor receptor is an agent that stimulates CD28. In some embodiments, the agent that stimulates a CD3/TCR complex is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand). In some embodiments, the agent that stimulates a costimulatory molecule and/or growth factor receptor is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand). In some embodiments, the agent that stimulates a CD3/TCR complex does not comprise a bead. In some embodiments, the agent that stimulates a costimulatory molecule and/or growth factor receptor does not comprise a bead. In some embodiments, the agent that stimulates a CD3/TCR complex comprises an anti-CD3 antibody. In some embodiments, the agent that stimulates a costimulatory molecule and/or growth factor receptor comprises an anti-CD28 antibody. In some embodiments, the agent that stimulates a CD3/TCR complex comprises an anti-CD3 antibody covalently attached to a colloidal polymeric nanomatrix. In some embodiments, the agent that stimulates CD3 comprises one or more of a CD3 or TCR antigen binding domain, such as but not limited to an anti-CD3 or anti-TCR antibody or an antibody fragment comprising one or more CDRs, heavy chain, and/or light chain thereof-such as but not limited to an anti-CD3 or anti-TCR antibody provided in Table 27. In some embodiments, the agent that stimulates a costimulatory molecule and/or growth factor receptor comprises an anti-CD28 antibody covalently attached to a colloidal polymeric nanomatrix. In some embodiments, the agent that stimulates a costimulatory molecule and/or growth factor receptor is an agent that stimulates CD28, ICOS, CD27, CD25, 4-1BB, IL6RA, IL6RB, or CD2. In some embodiments, the agent that stimulates a costimulatory molecule and/or growth factor receptor comprises one or more of a CD28, ICOS, CD27, CD25, 4-1BB, IL6RB, and/or CD2 antigen binding domain, such as but not limited to an anti-CD28, anti-ICOS, anti-CD27, anti-CD25, anti-4-1BB, anti-IL6RA, anti-IL6RB, or anti-CD2 antibody or an antibody fragment comprising one or more CDRs, heavy chain, and/or light chain thereof-such as but not limited to an anti-CD28, anti-ICOS, anti-CD27, anti-CD25, anti-4-1BB, anti-IL6RA, anti-IL6RB, or anti-CD2 antibody provided in Table 27. In some embodiments, the agent that stimulates a CD3/TCR complex and the agent that stimulates a costimulatory molecule and/or growth factor receptor comprise T Cell TransAct™. In some embodiments, the agent that stimulates a CD3/TCR complex and the agent that stimulates a costimulatory molecule and/or growth factor receptor are comprised in a multispecific binding molecule. In some embodiments, the multispecific binding molecule comprises a CD3 antigen binding domain and a CD28 or CD2 antigen binding domain. In some embodiments, the multispecific binding molecules comprise one or more heavy and/or light chains-such as but not limited to the heavy and/or light chains provided in Table 28. In some embodiments, the multispecific binding molecule comprises a bispecific antibody. In some embodiments, the bispecific antibody is configured in any one of the schema provided in FIG. 50A. In some embodiments, the bispecific antibody is monovalent or bivalent. In some embodiments, the bispecific antibody comprises an Fc region. In some embodiments, the Fc region of the bispecific antibody is silenced. In some embodiments, the multispecific binding molecule comprises a plurality of bispecific antibodies. In some embodiments, one or more of the plurality of bispecific antibodies is monovalent. In some embodiments, one or more of the plurality of bispecific antibodies comprises an Fc region. In some embodiments, the Fc region of the one or more of the plurality of bispecific antibodies is silenced. In some embodiments, one or more of the plurality of bispecific antibodies are conjugated together into a multimer. In some embodiments, the multimer is configured in any one of the schema provided in FIG. 50B.

In some embodiments, the agent that stimulates a CD3/TCR complex does not comprise hydrogel. In some embodiments, the agent that stimulates a costimulatory molecule and/or growth factor receptor does not comprise hydrogel. In some embodiments, the agent that stimulates a CD3/TCR complex does not comprise alginate. In some embodiments, the agent that stimulates a costimulatory molecule and/or growth factor receptor does not comprise alginate.

In some embodiments, the agent that stimulates a CD3/TCR complex comprises hydrogel. In some embodiments, the agent that stimulates a costimulatory molecule and/or growth factor receptor comprises hydrogel. In some embodiments, the agent that stimulates a CD3/TCR complex comprises alginate. In some embodiments, the agent that stimulates a costimulatory molecule and/or growth factor receptor comprises alginate. In some embodiments, the agent that stimulates a CD3/TCR complex or the agent that stimulates a costimulatory molecule and/or growth factor receptor comprises MagCloudz™ from Quad Technologies.

In some embodiments, step (i) increases the percentage of CAR-expressing cells in the population of cells from step (iii), for example, the population of cells from step (iii) shows a higher percentage of CAR-expressing cells (for example, at least 10, 20, 30, 40, 50, or 60% higher), compared with cells made by an otherwise similar method without step (i).

In some embodiments, the percentage of naïve cells, for example, naïve T cells, for example, CD45RA+CD45RO− CCR7+ T cells, in the population of cells from step (iii) is the same as the percentage of naïve cells, for example, naïve T cells, for example, CD45RA+CD45RO− CCR7+ cells, in the population of cells at the beginning of step (i). In some embodiments, the percentage of naïve cells, for example, naïve T cells, for example, CD45RA+CD45RO− CCR7+ T cells, in the population of cells from step (iii) differs by no more than 3, 4, 5, 6, 7, 8, 9, 10, 11, or 12% from the percentage of naïve cells, for example, naïve T cells, for example, CD45RA+CD45RO− CCR7+ cells, in the population of cells at the beginning of step (i). In some embodiments, the percentage of naïve cells, for example, naïve T cells, for example, CD45RA+CD45RO− CCR7+ T cells, in the population of cells from step (iii) differs by no more than 5 or 10% from the percentage of naïve cells, for example, naïve T cells, for example, CD45RA+CD45RO− CCR7+ cells, in the population of cells at the beginning of step (i).

In some embodiments, the population of cells from step (iii) shows a higher percentage of naïve cells, for example, naïve T cells, for example, CD45RA+CD45RO− CCR7+ T cells (for example, at least 10, 11, 12, 13, 14, 15, 16, 17, 18, 19, 20, 25, 30, 35, or 40% higher), compared with cells made by an otherwise similar method in which step (iii) is performed more than 26 hours after the beginning of step (i), for example, more than 5, 6, 7, 8, 9, 10, 11, or 12 days after the beginning of step (i). In some embodiments, the population of cells from step (iii) shows a higher percentage of naïve cells, for example, naïve T cells, for example, CD45RA+CD45RO− CCR7+ T cells (for example, at least 10, 11, 12, 13, 14, 15, 16, 17, 18, 19, 20, 25, 30, 35, or 40% higher), compared with cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the percentage of central memory cells, for example, central memory T cells, for example, CD95+ central memory T cells, in the population of cells from step (iii) is the same as the percentage of central memory cells, for example, central memory T cells, for example, CD95+ central memory T cells, in the population of cells at the beginning of step (i). In some embodiments, the percentage of central memory cells, for example, central memory T cells, for example, CD95+ central memory T cells, in the population of cells from step (iii) differs by no more than 3, 4, 5, 6, 7, 8, 9, 10, 11, or 12% from the percentage of central memory cells, for example, central memory T cells, for example, CD95+ central memory T cells, in the population of cells at the beginning of step (i). In some embodiments, the percentage of central memory cells, for example, central memory T cells, for example, CD95+ central memory T cells, in the population of cells from step (iii) differs by no more than 5 or 10% from the percentage of central memory cells, for example, central memory T cells, for example, CD95+ central memory T cells, in the population of cells at the beginning of step (i).

In some embodiments, the population of cells from step (iii) shows a lower percentage of central memory cells, for example, central memory T cells, for example, CD95+ central memory T cells (for example, at least 10, 11, 12, 13, 14, 15, 16, 17, 18, 19, 20, 25, 30, 35, or 40% lower), compared with cells made by an otherwise similar method in which step (iii) is performed more than 26 hours after the beginning of step (i), for example, more than 5, 6, 7, 8, 9, 10, 11, or 12 days after the beginning of step (i). In some embodiments, the population of cells from step (iii) shows a lower percentage of central memory cells, for example, central memory T cells, for example, CD95+ central memory T cells (for example, at least 10, 11, 12, 13, 14, 15, 16, 17, 18, 19, 20, 25, 30, 35, or 40% lower), compared with cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the percentage of stem memory T cells, for example, CD45RA+CD95+IL-2 receptor β+CCR7+CD62L+ T cells, in the population of cells from step (iii) is increased, as compared to the percentage of stem memory T cells, for example, CD45RA+CD95+IL-2 receptor β+CCR7+CD62L+ T cells, in the population of cells at the beginning of step (i). In some embodiments, the percentage of CAR-expressing stem memory T cells, for example, CAR-expressing CD45RA+CD95+IL-2 receptor β+CCR7+CD62L+ T cells, in the population of cells from step (iii) is increased, as compared to the percentage of CAR-expressing stem memory T cells, for example, CAR-expressing CD45RA+CD95+IL-2 receptor β+CCR7+CD62L+ T cells, in the population of cells at the beginning of step (i). In some embodiments, the percentage of stem memory T cells, for example, CD45RA+CD95+IL-2 receptor β+CCR7+CD62L+ T cells, in the population of cells from step (iii) is higher than the percentage of stem memory T cells, for example, CD45RA+CD95+IL-2 receptor β+CCR7+CD62L+ T cells, in cells made by an otherwise similar method in which step (iii) is performed more than 26 hours after the beginning of step (i), for example, more than 5, 6, 7, 8, 9, 10, 11, or 12 days after the beginning of step (i). In some embodiments, the percentage of CAR-expressing stem memory T cells, for example, CAR-expressing CD45RA+CD95+IL-2 receptor β+CCR7+CD62L+ T cells, in the population of cells from step (iii) is higher than the percentage of CAR-expressing stem memory T cells, for example, CAR-expressing CD45RA+CD95+IL-2 receptor β+CCR7+CD62L+ T cells, in cells made by an otherwise similar method in which step (iii) is performed more than 26 hours after the beginning of step (i), for example, more than 5, 6, 7, 8, 9, 10, 11, or 12 days after the beginning of step (i). In some embodiments, the percentage of stem memory T cells, for example, CD45RA+CD95+IL-2 receptor β+CCR7+CD62L+ T cells, in the population of cells from step (iii) is higher than the percentage of stem memory T cells, for example, CD45RA+CD95+IL-2 receptor β+CCR7+CD62L+ T cells, in cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the percentage of CAR-expressing stem memory T cells, for example, CAR-expressing CD45RA+CD95+IL-2 receptor β+CCR7+CD62L+ T cells, in the population of cells from step (iii) is higher than the percentage of CAR-expressing stem memory T cells, for example, CAR-expressing CD45RA+CD95+IL-2 receptor β+CCR7+CD62L+ T cells, in cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the median GeneSetScore (Up TEM vs. Down TSCM) of the population of cells from step (iii) is about the same as or differs by no more than (for example, increased by no more than) about 25, 50, 75, 100, or 125% from the median GeneSetScore (Up TEM vs. Down TSCM) of the population of cells at the beginning of step (i). In some embodiments, the median GeneSetScore (Up TEM vs. Down TSCM) of the population of cells from step (iii) is lower (for example, at least about 100, 150, 200, 250, or 300% lower) than the median GeneSetScore (Up TEM vs. Down TSCM) of cells made by an otherwise similar method in which step (iii) is performed more than 26 hours after the beginning of step (i), for example, more than 5, 6, 7, 8, 9, 10, 11, or 12 days after the beginning of step (i). In some embodiments, the median GeneSetScore (Up TEM vs. Down TSCM) of the population of cells from step (iii) is lower (for example, at least about 100, 150, 200, 250, or 300% lower) than the median GeneSetScore (Up TEM vs. Down TSCM) of cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the median GeneSetScore (Up Treg vs. Down Teff) of the population of cells from step (iii) is about the same as or differs by no more than (for example, increased by no more than) about 25, 50, 100, 150, or 200% from the median GeneSetScore (Up Treg vs. Down Teff) of the population of cells at the beginning of step (i). In some embodiments, the median GeneSetScore (Up Treg vs. Down Teff) of the population of cells from step (iii) is lower (for example, at least about 50, 100, 125, 150, or 175% lower) than the median GeneSetScore (Up Treg vs. Down Teff) of cells made by an otherwise similar method in which step (iii) is performed more than 26 hours after the beginning of step (i), for example, more than 5, 6, 7, 8, 9, 10, 11, or 12 days after the beginning of step (i). In some embodiments, the median GeneSetScore (Up Treg vs. Down Teff) of the population of cells from step (iii) is lower (for example, at least about 50, 100, 125, 150, or 175% lower) than the median GeneSetScore (Up Treg vs. Down Teff) of cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the median GeneSetScore (Down stemness) of the population of cells from step (iii) is about the same as or differs by no more than (for example, increased by no more than) about 25, 50, 100, 150, 200, or 250% from the median GeneSetScore (Down stemness) of the population of cells at the beginning of step (i). In some embodiments, the median GeneSetScore (Down stemness) of the population of cells from step (iii) is lower (for example, at least about 50, 100, or 125% lower) than the median GeneSetScore (Down stemness) of cells made by an otherwise similar method in which step (iii) is performed more than 26 hours after the beginning of step (i), for example, more than 5, 6, 7, 8, 9, 10, 11, or 12 days after the beginning of step (i). In some embodiments, the median GeneSetScore (Down stemness) of the population of cells from step (iii) is lower (for example, at least about 50, 100, or 125% lower) than the median GeneSetScore (Down stemness) of cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the median GeneSetScore (Up hypoxia) of the population of cells from step (iii) is about the same as or differs by no more than (for example, increased by no more than) about 125, 150, 175, or 200% from the median GeneSetScore (Up hypoxia) of the population of cells at the beginning of step (i). In some embodiments, the median GeneSetScore (Up hypoxia) of the population of cells from step (iii) is lower (for example, at least about 40, 50, 60, 70, or 80% lower) than the median GeneSetScore (Up hypoxia) of cells made by an otherwise similar method in which step (iii) is performed more than 26 hours after the beginning of step (i), for example, more than 5, 6, 7, 8, 9, 10, 11, or 12 days after the beginning of step (i). In some embodiments, the median GeneSetScore (Up hypoxia) of the population of cells from step (iii) is lower (for example, at least about 40, 50, 60, 70, or 80% lower) than the median GeneSetScore (Up hypoxia) of cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the median GeneSetScore (Up autophagy) of the population of cells from step (iii) is about the same as or differs by no more than (for example, increased by no more than) about 180, 190, 200, or 210% from the median GeneSetScore (Up autophagy) of the population of cells at the beginning of step (i). In some embodiments, the median GeneSetScore (Up autophagy) of the population of cells from step (iii) is lower (for example, at least 20, 30, or 40% lower) than the median GeneSetScore (Up autophagy) of cells made by an otherwise similar method in which step (iii) is performed more than 26 hours after the beginning of step (i), for example, more than 5, 6, 7, 8, 9, 10, 11, or 12 days after the beginning of step (i). In some embodiments, the median GeneSetScore (Up autophagy) of the population of cells from step (iii) is lower (for example, at least 20, 30, or 40% lower) than the median GeneSetScore (Up autophagy) of cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii), after being incubated with a cell expressing an antigen recognized by the CAR, secretes IL-2 at a higher level (for example, at least 2, 4, 6, 8, 10, 12, or 14-fold higher) than cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days, for example, as assessed using methods described in Example 8 with respect to FIGS. 29C-29D.

In some embodiments, the population of cells from step (iii), after being administered in vivo, persists longer or expands at a higher level (for example, at least 20, 25, 30, 35, 40, 45, 50, 55, 60, 65, 70, 75, 80, 85, or 90% higher) (for example, as assessed using methods described in Example 1 with respect to FIG. 4C), compared with cells made by an otherwise similar method in which step (iii) is performed more than 26 hours after the beginning of step (i), for example, more than 5, 6, 7, 8, 9, 10, 11, or 12 days after the beginning of step (i). In some embodiments, the population of cells from step (iii), after being administered in vivo, persists longer or expands at a higher level (for example, at least 20, 25, 30, 35, 40, 45, 50, 55, 60, 65, 70, 75, 80, 85, or 90% higher) (for example, as assessed using methods described in Example 1 with respect to FIG. 4C), compared with cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii), after being administered in vivo, shows a stronger anti-tumor activity (for example, a stronger anti-tumor activity at a low dose, for example, a dose no more than 0.15×10⁶, 0.2×10⁶, 0.25×10⁶, or 0.3×10⁶ viable CAR-expressing cells) than cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii) are not expanded, for example, as assessed by the number of living cells, compared to the population of cells at the beginning of step (i). In some embodiments, the population of cells from step (iii) decreases from the number of living cells in the population of cells at the beginning of step (i), for example, as assessed by the number of living cells. In some embodiments, the population of cells from step (iii) are expanded by no more than 5, 6, 7, 8, 9, 10, 11, 12, 13, 14, 15, 16, 17, 18, 19, 20, 25, 30, 35, or 40%, for example, no more than 10%, for example, as assessed by the number of living cells, compared to the population of cells at the beginning of step (i). In some embodiments, the population of cells from step (iii) are not expanded, or expanded by less than 0.5, 1, 1.5, or 2 hours, for example, less than 1 or 1.5 hours, compared to the population of cells at the beginning of step (i).

In some embodiments, steps (i) and (ii) are performed in cell media (for example, serum-free media) comprising IL-2, IL-15 (for example, hetIL-15 (IL15/sIL-15Ra)), IL-6 (for example, IL-6/sIL-6Ra), a LSD1 inhibitor, or a MALT1 inhibitor. In some embodiments, steps (i) and (ii) are performed in cell media (for example, serum-free media) comprising IL-7, IL-21, or a combination thereof. In some embodiments, steps (i) and (ii) are performed in cell media (for example, serum-free media) comprising IL-2, IL-15 (for example, hetIL-15 (IL15/sIL-15Ra)), IL-21, IL-7, IL-6 (for example, IL-6/sIL-6Ra), a LSD1 inhibitor, a MALT1 inhibitor, or a combination thereof. In some embodiments, step (i) is performed in cell media (for example, serum-free media) comprising IL-2, IL-15 (for example, hetIL-15 (IL15/sIL-15Ra)), IL-6 (for example, IL-6/sIL-6Ra), a LSD1 inhibitor, or a MALT1 inhibitor. In some embodiments, step (ii) is performed in cell media (for example, serum-free media) comprising IL-2, IL-15 (for example, hetIL-15 (IL15/sIL-15Ra)), IL-6 (for example, IL-6/sIL-6Ra), a LSD1 inhibitor, or a MALT1 inhibitor. In some embodiments, step (i) is performed in cell media (for example, serum-free media) comprising IL-7, IL-21, or a combination thereof. In some embodiments, step (ii) is performed in cell media (for example, serum-free media) comprising IL-7, IL-21, or a combination thereof. In some embodiments, step (i) is performed in cell media (for example, serum-free media) comprising IL-2, IL-15 (for example, hetIL-15 (IL15/sIL-15Ra)), IL-21, IL-7, IL-6 (for example, IL-6/sIL-6Ra), a LSD1 inhibitor, a MALT1 inhibitor, or a combination thereof. In some embodiments, step (ii) is performed in cell media (for example, serum-free media) comprising IL-2, IL-15 (for example, hetIL-15 (IL15/sIL-15Ra)), IL-21, IL-7, IL-6 (for example, IL-6/sIL-6Ra), a LSD1 inhibitor, a MALT1 inhibitor, or a combination thereof. In some embodiments, the cell media is a serum-free media comprising a serum replacement. In some embodiments, the serum replacement is CTS™ Immune Cell Serum Replacement (ICSR).

In some embodiments, the aforementioned methods further comprise prior to step (i): (iv) receiving a fresh leukapheresis product (or an alternative source of hematopoietic tissue such as a fresh whole blood product, a fresh bone marrow product, or a fresh tumor or organ biopsy or removal (for example, a fresh product from thymectomy)) from an entity, for example, a laboratory, hospital, or healthcare provider.

In some embodiments, the aforementioned methods further comprise prior to step (i): (v) isolating the population of cells (for example, T cells, for example, CD8+ and/or CD4+ T cells) contacted in step (i) from a fresh leukapheresis product (or an alternative source of hematopoietic tissue such as a fresh whole blood product, a fresh bone marrow product, or a fresh tumor or organ biopsy or removal (for example, a fresh product from thymectomy)). In some embodiments, step (iii) is performed no later than 35, 36, or 48 hours after the beginning of step (v), for example, no later than 27, 28, 29, 30, 31, 32, 33, 34, 35, 36, 37, 38, 39, 40, 41, 42, 43, 44, 45, 46, 47, or 48 hours after the beginning of step (v), for example, no later than 30, 36, or 48 hours after the beginning of step (v). In some embodiments, the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

In some embodiments, the aforementioned methods further comprise prior to step (i): receiving cryopreserved T cells isolated from a leukapheresis product (or an alternative source of hematopoietic tissue such as cryopreserved T cells isolated from whole blood, bone marrow, or tumor or organ biopsy or removal (for example, thymectomy)) from an entity, for example, a laboratory, hospital, or healthcare provider.

In some embodiments, the aforementioned methods further comprise prior to step (i): (iv) receiving a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)) from an entity, for example, a laboratory, hospital, or healthcare provider.

In some embodiments, the aforementioned methods further comprise prior to step (i): (v) isolating the population of cells (for example, T cells, for example, CD8+ and/or CD4+ T cells) contacted in step (i) from a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)). In some embodiments, step (iii) is performed no later than 35, 36, or 48 hours after the beginning of step (v), for example, no later than 27, 28, 29, 30, 31, 32, 33, 34, 35, 36, 37, 38, 39, 40, 41, 42, 43, 44, 45, 46, 47, or 48 hours after the beginning of step (v), for example, no later than 30, 36, or 38 hours after the beginning of step (v). In some embodiments, the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

In some embodiments, this invention features a method of making a population of cells (for example, T cells) that express a chimeric antigen receptor (CAR), the method comprising: (1) contacting a population of cells (for example, T cells, for example, T cells isolated from a frozen leukapheresis product) with a cytokine chosen from IL-2, IL-7, IL-15, IL-21, IL-6, or a combination thereof, (2) contacting the population of cells (for example, T cells) with a nucleic acid molecule (for example, a DNA or RNA molecule) encoding the CAR, thereby providing a population of cells (for example, T cells) comprising the nucleic acid molecule, and (3) harvesting the population of cells (for example, T cells) for storage (for example, reformulating the population of cells in cryopreservation media) or administration, wherein: (a) step (2) is performed together with step (1) or no later than 5 hours after the beginning of step (1), for example, no later than 1, 2, 3, 4, or 5 hours after the beginning of step (1), and step (3) is performed no later than 26 hours after the beginning of step (1), for example, no later than 22, 23, 24, or 25 hours after the beginning of step (1), for example, no later than 24 hours after the beginning of step (1), or (b) the population of cells from step (3) are not expanded, or expanded by no more than 5, 10, 15, 20, 25, 30, 35, or 40%, for example, no more than 10%, for example, as assessed by the number of living cells, compared to the population of cells at the beginning of step (1). In some embodiments, the nucleic acid molecule in step (2) is a DNA molecule. In some embodiments, the nucleic acid molecule in step (2) is an RNA molecule. In some embodiments, the nucleic acid molecule in step (2) is on a viral vector, for example, a viral vector chosen from a lentivirus vector, an adenoviral vector, or a retrovirus vector. In some embodiments, the nucleic acid molecule in step (2) is on a non-viral vector. In some embodiments, the nucleic acid molecule in step (2) is on a plasmid. In some embodiments, the nucleic acid molecule in step (2) is not on any vector. In some embodiments, step (2) comprises contacting, optionally transducing, the population of cells (for example, T cells) with a viral vector comprising a nucleic acid molecule encoding the CAR. In some embodiments, step (2) further comprises contacting the population of cells (for example, T cells) with shRNA that targets Tet2 comprising (A) a sense strand comprising a Tet2 target sequence and (B) an antisense strand complementary to the sense strand in whole or in part or a vector encoding the shRNA. In some embodiments, sense strand comprises the Tet2 target sequence GGGTAAGCCAAGAAAGAAA (SEQ ID NO: 418). In some embodiments, the anti-sense strand comprises the reverse complement thereof, i.e. TTTCTTTCTTGGCTTACCC (SEQ ID NO: 419). In some embodiments, the vector encoding the shRNA is the same or different from the vector encoding the CAR. In some embodiments, the vector encoding the shRNA sequence comprises promoter (such as but not limited to a U6 promoter), a sense strand comprising a Tet2 target sequence, a loop, an anti-sense strand complementary to the sense strand in whole or in part, and, optionally, a polyT tail, e.g. the sequences in Table 29.

In some embodiments, step (2) is performed together with step (1). In some embodiments, step (2) is performed no later than 5 hours after the beginning of step (1). In some embodiments, step (2) is performed no later than 1, 2, 3, 4, or 5 hours after the beginning of step (1). In some embodiments, step (3) is performed no later than 26 hours after the beginning of step (1). In some embodiments, step (3) is performed no later than 22, 23, 24, or 25 hours after the beginning of step (1). In some embodiments, step (3) is performed no later than 24 hours after the beginning of step (1).

In some embodiments, the population of cells from step (3) are not expanded, for example, as assessed by the number of living cells, compared to the population of cells at the beginning of step (1). In some embodiments, the population of cells from step (3) are expanded by no more than 5, 6, 7, 8, 9, 10, 11, 12, 13, 14, 15, 16, 17, 18, 19, 20, 25, 30, 35, or 40%, for example, as assessed by the number of living cells, compared to the population of cells at the beginning of step (1). In some embodiments, the population of cells from step (3) are expanded by no more than 10%, for example, as assessed by the number of living cells, compared to the population of cells at the beginning of step (1).

In some embodiments, step (1) comprises contacting the population of cells (for example, T cells) with IL-2. In some embodiments, step (1) comprises contacting the population of cells (for example, T cells) with IL-7. In some embodiments, step (1) comprises contacting the population of cells (for example, T cells) with IL-15 (for example, hetIL-15 (IL15/sIL-15Ra)). In some embodiments, step (1) comprises contacting the population of cells (for example, T cells) with IL-21. In some embodiments, step (1) comprises contacting the population of cells (for example, T cells) with IL-6 (for example, IL-6/sIL-6Ra). In some embodiments, step (1) comprises contacting the population of cells (for example, T cells) with IL-2 and IL-7. In some embodiments, step (1) comprises contacting the population of cells (for example, T cells) with IL-2 and IL-15 (for example, hetIL-15 (IL15/sIL-15Ra)). In some embodiments, step (1) comprises contacting the population of cells (for example, T cells) with IL-2 and IL-21. In some embodiments, step (1) comprises contacting the population of cells (for example, T cells) with IL-2 and IL-6 (for example, IL-6/sIL-6Ra). In some embodiments, step (1) comprises contacting the population of cells (for example, T cells) with IL-7 and IL-15 (for example, hetIL-15 (IL15/sIL-15Ra)). In some embodiments, step (1) comprises contacting the population of cells (for example, T cells) with IL-7 and IL-21. In some embodiments, step (1) comprises contacting the population of cells (for example, T cells) with IL-7 and IL-6 (for example, IL-6/sIL-6Ra). In some embodiments, step (1) comprises contacting the population of cells (for example, T cells) with IL-15 (for example, hetIL-15 (IL15/sIL-15Ra)) and IL-21. In some embodiments, step (1) comprises contacting the population of cells (for example, T cells) with IL-15 (for example, hetIL-15 (IL15/sIL-15Ra)) and IL-6 (for example, IL-6/sIL-6Ra). In some embodiments, step (1) comprises contacting the population of cells (for example, T cells) with IL-21 and IL-6 (for example, IL-6/sIL-6Ra). In some embodiments, step (1) comprises contacting the population of cells (for example, T cells) with IL-7, IL-15 (for example, hetIL-15 (IL15/sIL-15Ra)), and IL-21.

In some embodiments, the population of cells from step (3) shows a higher percentage of naïve cells among CAR-expressing cells (for example, at least 10, 11, 12, 13, 14, 15, 16, 17, 18, 19, 20, 25, 30, 35, or 40% higher), compared with cells made by an otherwise similar method which further comprises contacting the population of cells with, for example, an anti-CD3 antibody.

In some embodiments, the percentage of naïve cells, for example, naïve T cells, for example, CD45RA+CD45RO− CCR7+ T cells, in the population of cells from step (3) is the same as the percentage of naïve cells, for example, naïve T cells, for example, CD45RA+CD45RO− CCR7+ cells, in the population of cells at the beginning of step (1). In some embodiments, the percentage of naïve cells, for example, naïve T cells, for example, CD45RA+CD45RO− CCR7+ T cells, in the population of cells from step (3) differs by no more than 3, 4, 5, 6, 7, 8, 9, 10, 11, or 12% from the percentage of naïve cells, for example, naïve T cells, for example, CD45RA+CD45RO− CCR7+ cells, in the population of cells at the beginning of step (1). In some embodiments, the percentage of naïve cells, for example, naïve T cells, for example, CD45RA+CD45RO− CCR7+ T cells, in the population of cells from step (3) differs by no more than 5 or 10% from the percentage of naïve cells, for example, naïve T cells, for example, CD45RA+CD45RO− CCR7+ cells, in the population of cells at the beginning of step (1). In some embodiments, the percentage of naïve cells, for example, naïve T cells, for example, CD45RA+CD45RO− CCR7+ T cells, in the population of cells from step (3) is increased as compared to the percentage of naïve cells, for example, naïve T cells, for example, CD45RA+CD45RO− CCR7+ cells, in the population of cells at the beginning of step (1). In some embodiments, the percentage of naïve cells, for example, naïve T cells, for example, CD45RA+CD45RO− CCR7+ T cells, in the population of cells from step (3) is increased by at least 10, 11, 12, 13, 14, 15, 16, 17, 18, 19, or 20%, as compared to the percentage of naïve cells, for example, naïve T cells, for example, CD45RA+CD45RO− CCR7+ cells, in the population of cells at the beginning of step (1). In some embodiments, the percentage of naïve cells, for example, naïve T cells, for example, CD45RA+CD45RO− CCR7+ T cells, in the population of cells from step (3) is increased by at least 10 or 20%, as compared to the percentage of naïve cells, for example, naïve T cells, for example, CD45RA+CD45RO− CCR7+ cells, in the population of cells at the beginning of step (1).

In some embodiments, the population of cells from step (3) shows a higher percentage of naïve cells, for example, naïve T cells, for example, CD45RA+CD45RO− CCR7+ T cells (for example, at least 10, 11, 12, 13, 14, 15, 16, 17, 18, 19, 20, 25, 30, 35, or 40% higher), compared with cells made by an otherwise similar method in which step (3) is performed more than 26 hours after the beginning of step (1), for example, more than 5, 6, 7, 8, 9, 10, 11, or 12 days after the beginning of step (1). In some embodiments, the population of cells from step (3) shows a higher percentage of naïve cells, for example, naïve T cells, for example, CD45RA+CD45RO− CCR7+ T cells (for example, at least 10, 11, 12, 13, 14, 15, 16, 17, 18, 19, 20, 25, 30, 35, or 40% higher), compared with cells made by an otherwise similar method which further comprises, after step (2) and prior to step (3), expanding the population of cells (for example, T cells) in vitro for more than 3 days, for example, for 5, 6, 7, 8 or 9 days.

In some embodiments, the percentage of central memory cells, for example, central memory T cells, for example, CD95+ central memory T cells, in the population of cells from step (3) is the same as the percentage of central memory cells, for example, central memory T cells, for example, CD95+ central memory T cells, in the population of cells at the beginning of step (i). In some embodiments, the percentage of central memory cells, for example, central memory T cells, for example, CD95+ central memory T cells, in the population of cells from step (3) differs by no more than 3, 4, 5, 6, 7, 8, 9, 10, 11, or 12% from the percentage of central memory cells, for example, central memory T cells, for example, CD95+ central memory T cells, in the population of cells at the beginning of step (i). In some embodiments, the percentage of central memory cells, for example, central memory T cells, for example, CD95+ central memory T cells, in the population of cells from step (3) differs by no more than 5 or 10% from the percentage of central memory cells, for example, central memory T cells, for example, CD95+ central memory T cells, in the population of cells at the beginning of step (i). In some embodiments, the percentage of central memory cells, for example, central memory T cells, for example, CD95+ central memory T cells, in the population of cells from step (3) is decreased as compared to the percentage of central memory cells, for example, central memory T cells, for example, CD95+ central memory T cells, in the population of cells at the beginning of step (1). In some embodiments, the percentage of central memory cells, for example, central memory T cells, for example, CD95+ central memory T cells, in the population of cells from step (3) is decreased by at least 10 or 20%, as compared to the percentage of central memory cells, for example, central memory T cells, for example, CD95+ central memory T cells, in the population of cells at the beginning of step (1). In some embodiments, the percentage of central memory cells, for example, central memory T cells, for example, CD95+ central memory T cells, in the population of cells from step (3) is decreased by at least 10, 11, 12, 13, 14, 15, 16, 17, 18, 19, or 20%, as compared to the percentage of central memory cells, for example, central memory T cells, for example, CD95+ central memory T cells, in the population of cells at the beginning of step (1).

In some embodiments, the population of cells from step (3) shows a lower percentage of central memory cells, for example, central memory T cells, for example, CD95+ central memory T cells (for example, at least 10, 11, 12, 13, 14, 15, 16, 17, 18, 19, 20, 25, 30, 35, or 40% lower), compared with cells made by an otherwise similar method in which step (3) is performed more than 26 hours after the beginning of step (1), for example, more than 5, 6, 7, 8, 9, 10, 11, or 12 days after the beginning of step (1). In some embodiments, the population of cells from step (3) shows a lower percentage of central memory cells, for example, central memory T cells, for example, CD95+ central memory T cells (for example, at least 10, 11, 12, 13, 14, 15, 16, 17, 18, 19, 20, 25, 30, 35, or 40% lower), compared with cells made by an otherwise similar method which further comprises, after step (2) and prior to step (3), expanding the population of cells (for example, T cells) in vitro for more than 3 days, for example, for 5, 6, 7, 8 or 9 days.

In some embodiments, the population of cells from step (3), after being administered in vivo, persists longer or expands at a higher level (for example, at least 20, 25, 30, 35, 40, 45, 50, 55, 60, 65, 70, 75, 80, 85, or 90% higher) (for example, as assessed using methods described in Example 1 with respect to FIG. 4C), compared with cells made by an otherwise similar method in which step (3) is performed more than 26 hours after the beginning of step (1), for example, more than 5, 6, 7, 8, 9, 10, 11, or 12 days after the beginning of step (1). In some embodiments, the population of cells from step (3), after being administered in vivo, persists longer or expands at a higher level (for example, at least 20, 25, 30, 35, 40, 45, 50, 55, 60, 65, 70, 75, 80, 85, or 90% higher) (for example, as assessed using methods described in Example 1 with respect to FIG. 4C), compared with cells made by an otherwise similar method which further comprises, after step (2) and prior to step (3), expanding the population of cells (for example, T cells) in vitro for more than 3 days, for example, for 5, 6, 7, 8 or 9 days.

In some embodiments, the population of cells from step (3) are not expanded, for example, as assessed by the number of living cells, compared to the population of cells at the beginning of step (1). In some embodiments, the population of cells from step (3) are expanded by no more than 5, 6, 7, 8, 9, 10, 11, 12, 13, 14, 15, 16, 17, 18, 19, 20, 25, 30, 35, or 40%, for example, as assessed by the number of living cells, compared to the population of cells at the beginning of step (1). In some embodiments, the population of cells from step (3) are expanded by no more than 10%, for example, as assessed by the number of living cells, compared to the population of cells at the beginning of step (1). In some embodiments, the number of living cells in the population of cells from step (3) decreases from the number of living cells in the population of cells at the beginning of step (1), for example, as assessed by the number of living cells.

In some embodiments, the population of cells from step (3) are not expanded compared to the population of cells at the beginning of step (1), for example, as assessed by the number of living cells. In some embodiments, the population of cells from step (3) are expanded by less than 0.5, 1, 1.5, or 2 hours, for example, less than 1 or 1.5 hours, compared to the population of cells at the beginning of step (1).

In some embodiments, the population of cells is not contacted in vitro with (A) an agent that stimulates a CD3/TCR complex and/or (B) an agent that stimulates a costimulatory molecule and/or growth factor receptor on the surface of the cells, or if contacted, the contacting step is less than 2 hours, for example, no more than 1 or 1.5 hours. In some embodiments, the agent that stimulates a CD3/TCR complex is an agent that stimulates CD3 (for example, an anti-CD3 antibody). In some embodiments, the agent that stimulates a costimulatory molecule and/or growth factor receptor is an agent that stimulates CD28, ICOS, CD27, HVEM, LIGHT, CD40, 4-1BB, OX40, DR3, GITR, CD30, TIM1, CD2, CD226, or any combination thereof. In some embodiments, the agent that stimulates a costimulatory molecule and/or growth factor receptor is an agent that stimulates CD28. In some embodiments, the agent that stimulates a CD3/TCR complex or the agent that stimulates a costimulatory molecule and/or growth factor receptor is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand).

In some embodiments, steps (1) and/or (2) are performed in cell media comprising no more than 5, 4, 3, 2, 1, or 0% serum. In some embodiments, steps (1) and/or (2) are performed in cell media comprising no more than 2% serum. In some embodiments, steps (1) and/or (2) are performed in cell media comprising about 2% serum. In some embodiments, steps (1) and/or (2) are performed in cell media comprising a LSD1 inhibitor or a MALT1 inhibitor. In some embodiments, step (1) is performed in cell media comprising no more than 5, 4, 3, 2, 1, or 0% serum. In some embodiments, step (1) is performed in cell media comprising no more than 2% serum. In some embodiments, step (1) is performed in cell media comprising about 2% serum. In some embodiments, step (2) is performed in cell media comprising no more than 5, 4, 3, 2, 1, or 0% serum. In some embodiments, step (2) is performed in cell media comprising no more than 2% serum. In some embodiments, step (2) is performed in cell media comprising about 2% serum. In some embodiments, step (1) is performed in cell media comprising a LSD1 inhibitor or a MALT1 inhibitor. In some embodiments, step (2) is performed in cell media comprising a LSD1 inhibitor or a MALT1 inhibitor.

In some embodiments, the aforementioned methods further comprise prior to step (i): (iv) receiving a fresh leukapheresis product (or an alternative source of hematopoietic tissue such as a fresh whole blood product, a fresh bone marrow product, or a fresh tumor or organ biopsy or removal (for example, a fresh product from thymectomy)) from an entity, for example, a laboratory, hospital, or healthcare provider.

In some embodiments, the aforementioned methods further comprise prior to step (i): (v) isolating the population of cells (for example, T cells, for example, CD8+ and/or CD4+T cells) contacted in step (i) from a fresh leukapheresis product (or an alternative source of hematopoietic tissue such as a fresh whole blood product, a fresh bone marrow product, or a fresh tumor or organ biopsy or removal (for example, a fresh product from thymectomy)). In some embodiments, step (iii) is performed no later than 35, 36, or 48 hours after the beginning of step (v), for example, no later than 27, 28, 29, 30, 31, 32, 33, 34, 35, 36, 37, 38, 39, 40, 41, 42, 43, 44, 45, 46, 47, or 48 hours after the beginning of step (v), for example, no later than 30, 36, or 48 hours after the beginning of step (v). In some embodiments, the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

In some embodiments, the aforementioned methods further comprise prior to step (i): receiving cryopreserved T cells isolated from a leukapheresis product (or an alternative source of hematopoietic tissue such as cryopreserved T cells isolated from whole blood, bone marrow, or tumor or organ biopsy or removal (for example, thymectomy)) from an entity, for example, a laboratory, hospital, or healthcare provider.

In some embodiments, the aforementioned methods further comprise prior to step (i): (iv) receiving a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)) from an entity, for example, a laboratory, hospital, or healthcare provider.

In some embodiments, the aforementioned methods further comprise prior to step (i): (v) isolating the population of cells (for example, T cells, for example, CD8+ and/or CD4+ T cells) contacted in step (i) from a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)). In some embodiments, step (iii) is performed no later than 35, 36, or 48 hours after the beginning of step (v), for example, no later than 27, 28, 29, 30, 31, 32, 33, 34, 35, 36, 37, 38, 39, 40, 41, 42, 43, 44, 45, 46, 47, or 48 hours after the beginning of step (v), for example, no later than 30, 36, or 48 hours after the beginning of step (v). In some embodiments, the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

In some embodiments, the population of cells at the beginning of step (i) or step (1) has been enriched for IL6R-expressing cells (for example, cells that are positive for IL6Ra and/or IL6RB). In some embodiments, the population of cells at the beginning of step (i) or step (1) comprises no less than 40, 45, 50, 55, 60, 65, or 70% of IL6R-expressing cells (for example, cells that are positive for IL6Ra and/or IL6RB).

In some embodiments, steps (i) and (ii) or steps (1) and (2) are performed in cell media comprising IL-15 (for example, hetIL-15 (IL15/sIL-15Ra)). In some embodiments, IL-15 increases the ability of the population of cells to expand, for example, 10, 15, 20, or 25 days later. In some embodiments, IL-15 increases the percentage of IL6RB-expressing cells in the population of cells.

In some embodiments of the aforementioned methods, the methods are performed in a closed system. In some embodiments, T cell separation, activation, transduction, incubation, and washing are all performed in a closed system. In some embodiments of the aforementioned methods, the methods are performed in separate devices. In some embodiments, T cell separation, activation and transduction, incubation, and washing are performed in separate devices.

In some embodiments of the aforementioned methods, the methods further comprise adding an adjuvant or a transduction enhancement reagent in the cell culture medium to enhance transduction efficiency. In some embodiments, the adjuvant or transduction enhancement reagent comprises a cationic polymer. In some embodiments, the adjuvant or transduction enhancement reagent is chosen from: LentiBOOST™ (Sirion Biotech), vectofusin-1, F108 (Poloxamer 338 or Pluronic® F-38), protamine sulfate, hexadimethrine bromide (Polybrene), PEA, Pluronic F68, Pluronic F127, Synperonic or LentiTrans™. In some embodiments, the transduction enhancement reagent is LentiBOOST™ (Sirion Biotech). In some embodiments, the transduction enhancement reagent is F108 (Poloxamer 338 or Pluronic® F-38).

In some embodiments of the aforementioned methods, the transducing the population of cells (for example, T cells) with a viral vector comprises subjecting the population of cells and viral vector to a centrifugal force under conditions such that transduction efficiency is enhanced. In an embodiment, the cells are transduced by spinoculation.

In some embodiments of the aforementioned methods, cells (e.g., T cells) are activated and transduced in a cell culture flask comprising a gas-permeable membrane at the base that supports large media volumes without substantially compromising gas exchange. In some embodiments, cell growth is achieved by providing access, e.g., substantially uninterrupted access, to nutrients through convection.

In some embodiments of the aforementioned methods, the CAR comprises an antigen binding domain, a transmembrane domain, and an intracellular signaling domain.

In some embodiments, the antigen binding domain binds to an antigen chosen from: CD19, CD20, CD22, BCMA, mesothelin, EGFRVIII, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, Lewis Y, CD24, PDGFR-beta, SSEA-4, folate receptor alpha, ERBBs (for example, ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRα4, or a peptide of any of these antigens presented on MHC. In some embodiments, the antigen binding domain comprises a CDR, VH, VL, scFv or a CAR sequence disclosed herein. In some embodiments, the antigen binding domain comprises a VH and a VL, wherein the VH and VL are connected by a linker, optionally wherein the linker comprises the amino acid sequence of SEQ ID NO: 63 or 104.

In some embodiments, the transmembrane domain comprises a transmembrane domain of a protein chosen from the alpha, beta or zeta chain of T-cell receptor, CD28, CD3 epsilon, CD45, CD4, CD5, CD8, CD9, CD16, CD22, CD33, CD37, CD64, CD80, CD86, CD134, CD137 and CD154. In some embodiments, the transmembrane domain comprises a transmembrane domain of CD8. In some embodiments, the transmembrane domain comprises the amino acid sequence of SEQ ID NO: 6, or an amino acid sequence having at least about 85%, 90%, 95%, or 99% sequence identity thereof. In some embodiments, the nucleic acid molecule comprises a nucleic acid sequence encoding the transmembrane domain, wherein the nucleic acid sequence comprises the nucleic acid sequence of SEQ ID NO: 17, or a nucleic acid sequence having at least about 85%, 90%, 95%, or 99% sequence identity thereof.

In some embodiments, the antigen binding domain is connected to the transmembrane domain by a hinge region. In some embodiments, the hinge region comprises the amino acid sequence of SEQ ID NO: 2, 3, or 4, or an amino acid sequence having at least about 85%, 90%, 95%, or 99% sequence identity thereof. In some embodiments, the nucleic acid molecule comprises a nucleic acid sequence encoding the hinge region, wherein the nucleic acid sequence comprises the nucleic acid sequence of SEQ ID NO: 13, 14, or 15, or a nucleic acid sequence having at least about 85%, 90%, 95%, or 99% sequence identity thereof.

In some embodiments, the intracellular signaling domain comprises a primary signaling domain. In some embodiments, the primary signaling domain comprises a functional signaling domain derived from CD3 zeta, TCR zeta, FcR gamma, FcR beta, CD3 gamma, CD3 delta, CD3 epsilon, CD5, CD22, CD79a, CD79b, CD278 (ICOS), FcεRI, DAP10, DAP12, or CD66d. In some embodiments, the primary signaling domain comprises a functional signaling domain derived from CD3 zeta. In some embodiments, the primary signaling domain comprises the amino acid sequence of SEQ ID NO: 9 or 10, or an amino acid sequence having at least about 85%, 90%, 95%, or 99% sequence identity thereof. In some embodiments, the nucleic acid molecule comprises a nucleic acid sequence encoding the primary signaling domain, wherein the nucleic acid sequence comprises the nucleic acid sequence of SEQ ID NO: 20 or 21, or a nucleic acid sequence having at least about 85%, 90%, 95%, or 99% sequence identity thereof.

In some embodiments, the intracellular signaling domain comprises a costimulatory signaling domain. In some embodiments, the costimulatory signaling domain comprises a functional signaling domain derived from a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, or a ligand that specifically binds with CD83. In some embodiments, the costimulatory signaling domain comprises a functional signaling domain derived from 4-1BB. In some embodiments, the costimulatory signaling domain comprises the amino acid sequence of SEQ ID NO: 7, or an amino acid sequence having at least about 85%, 90%, 95%, or 99% sequence identity thereof. In some embodiments, the nucleic acid molecule comprises a nucleic acid sequence encoding the costimulatory signaling domain, wherein the nucleic acid sequence comprises the nucleic acid sequence of SEQ ID NO: 18, or a nucleic acid sequence having at least about 85%, 90%, 95%, or 99% sequence identity thereof.

In some embodiments, the intracellular signaling domain comprises a functional signaling domain derived from 4-1BB and a functional signaling domain derived from CD3 zeta. In some embodiments, the intracellular signaling domain comprises the amino acid sequence of SEQ ID NO: 7 (or an amino acid sequence having at least about 85%, 90%, 95%, or 99% sequence identity thereof) and the amino acid sequence of SEQ ID NO: 9 or 10 (or an amino acid sequence having at least about 85%, 90%, 95%, or 99% sequence identity thereof). In some embodiments, the intracellular signaling domain comprises the amino acid sequence of SEQ ID NO: 7 and the amino acid sequence of SEQ ID NO: 9 or 10.

In some embodiments, the CAR further comprises a leader sequence comprising the amino acid sequence of SEQ ID NO: 1.

In some embodiments, this invention features a population of CAR-expressing cells (for example, autologous or allogeneic CAR-expressing T cells or NK cells) made by any of the aforementioned methods or any other method disclosed herein. In some embodiments, disclosed herein is a pharmaceutical composition comprising a population of CAR-expressing cells disclosed herein and a pharmaceutically acceptable carrier.

In some embodiments, in the final CAR cell product manufactured using the methods described herein, the total amount of beads (e.g., CD4 beads, CD8 beads, and/or TransACT beads) is no more than 0.04, 0.05, 0.06, 0.07, 0.08, 0.09, 0.1, 0.2, 0.3, 0.4, or 0.5% of the total amount of beads added during the manufacturing process.

In some embodiments, this invention features a population of CAR-expressing cells (for example, autologous or allogeneic CAR-expressing T cells or NK cells) comprising one or more of the following characteristics: (a) about the same percentage of naïve cells, for example, naïve T cells, for example, CD45RO− CCR7+ T cells, as compared to the percentage of naïve cells, for example, naïve T cells, for example, CD45RO− CCR7+ cells, in the same population of cells prior to being engineered to express the CAR; (b) a change within about 5% to about 10% of naïve cells, for example, naïve T cells, for example, CD45RO− CCR7+ T cells, for example, as compared to the percentage of naïve cells, for example, naïve T cells, for example, CD45RO− CCR7+ cells, in the same population of cells prior to being engineered to express the CAR; (c) an increased percentage of naïve cells, for example, naïve T cells, for example, CD45RO− CCR7+ T cells, for example, increased by at least 1.2, 1.4, 1.6, 1.8, 2.0, 2.2, 2.4, 2.6, 2.8, or 3-fold, as compared to the percentage of naïve cells, for example, naïve T cells, for example, CD45RO− CCR7+ cells, in the same population of cells prior to being engineered to express the CAR; (d) about the same percentage of central memory cells, for example, central memory T cells, for example, CCR7+CD45RO+ T cells, as compared to the percentage of central memory cells, for example, central memory T cells, for example, CCR7+CD45RO+ T cells, in the same population of cells prior to being engineered to express the CAR; (e) a change within about 5% to about 10% of central memory cells, for example, central memory T cells, for example, CCR7+CD45RO+ T cells, as compared to the percentage of central memory cells, for example, central memory T cells, for example, CCR7+CD45RO+ T cells, in the same population of cells prior to being engineered to express the CAR; (f) a decreased percentage of central memory cells, for example, central memory T cells, for example, CCR7+CD45RO+ T cells, for example, decreased by at least 20, 25, 30, 35, 40, 45, or 50%, as compared to the percentage of central memory cells, for example, central memory T cells, for example, CCR7+CD45RO+ T cells, in the same population of cells prior to being engineered to express the CAR; (g) about the same percentage of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR; (h) a change within about 5% to about 10% of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR; or (i) an increased percentage of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR.

In some embodiments, this invention features a population of CAR-expressing cells (for example, autologous or allogeneic CAR-expressing T cells or NK cells), wherein: (a) the median GeneSetScore (Up TEM vs. Down TSCM) of the population of cells is about the same as or differs by no more than (for example, increased by no more than) about 25, 50, 75, 100, or 125% from the median GeneSetScore (Up TEM vs. Down TSCM) of the same population of cells prior to being engineered to express the CAR; (b) the median GeneSetScore (Up Treg vs. Down Teff) of the population of cells is about the same as or differs by no more than (for example, increased by no more than) about 25, 50, 100, 150, or 200% from the median GeneSetScore (Up Treg vs. Down Teff) of the population of cells prior to being engineered to express the CAR; (c) the median GeneSetScore (Down stemness) of the population of cells is about the same as or differs by no more than (for example, increased by no more than) about 25, 50, 100, 150, 200, or 250% from the median GeneSetScore (Down stemness) of the population of cells prior to being engineered to express the CAR; (d) the median GeneSetScore (Up hypoxia) of the population of cells is about the same as or differs by no more than (for example, increased by no more than) about 125, 150, 175, or 200% from the median GeneSetScore (Up hypoxia) of the population of cells prior to being engineered to express the CAR; or (e) the median GeneSetScore (Up autophagy) of the population of cells is about the same as or differs by no more than (for example, increased by no more than) about 180, 190, 200, or 210% from the median GeneSetScore (Up autophagy) of the population of cells prior to being engineered to express the CAR.

In some embodiments, this invention features a method of increasing an immune response in a subject, comprising administering a population of CAR-expressing cells disclosed herein or a pharmaceutical composition disclosed herein to the subject, thereby increasing an immune response in the subject.

In some embodiments, disclosed herein is a method of treating a cancer in a subject, comprising administering a population of CAR-expressing cells disclosed herein or a pharmaceutical composition disclosed herein to the subject, thereby treating the cancer in the subject. In some embodiments, the cancer is a solid cancer, for example, chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof. In some embodiments, the cancer is a liquid cancer, for example, chosen from: chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+large B-cell lymphoma, large B-cell lymphoma arising in HHV8-associated multicentric Castleman disease, primary effusion lymphoma, B-cell lymphoma, acute myeloid leukemia (AML), or unclassifiable lymphoma.

In some embodiments, the method further comprises administering a second therapeutic agent to the subject. In some embodiments, the second therapeutic agent is an anti-cancer therapeutic agent, for example, a chemotherapy, a radiation therapy, or an immune-regulatory therapy. In some embodiments, the second therapeutic agent is IL-15 (for example, hetIL-15 (IL15/sIL-15Ra)).

In some aspects, the disclosure features an antibody molecule that binds CD28, comprising a heavy chain variable region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3, wherein (i) the HCDR1, HCDR2, HCDR3, LCDR1, LCDR2, and LCDR3 comprise the amino acid sequences of SEQ ID NO: 538, 539, 540, 530, 531, and 532, respectively; (ii) the HCDR1, HCDR2, HCDR3, LCDR1, LCDR2, and LCDR3 comprise the amino acid sequences of SEQ ID NO: 541, 539, 540, 530, 531, and 532, respectively; (iii) the HCDR1, HCDR2, HCDR3, LCDR1, LCDR2, and LCDR3 comprise the amino acid sequences of SEQ ID NO: 542, 543, 540, 533, 534, and 535, respectively; or (iv) the HCDR1, HCDR2, HCDR3, LCDR1, LCDR2, and LCDR3 comprise the amino acid sequences of SEQ ID NO: 544, 545, 546, 536, 534, and 532, respectively.

In some aspects, the disclosure features an antibody molecule that binds CD28, comprising a heavy chain variable region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3, and an Fc region, wherein (i) the HCDR1, HCDR2, HCDR3, LCDR1, LCDR2, and LCDR3 comprise the amino acid sequences of SEQ ID NO: 538, 539, 540, 530, 531, and 532, respectively; (ii) the HCDR1, HCDR2, HCDR3, LCDR1, LCDR2, and LCDR3 comprise the amino acid sequences of SEQ ID NO: 541, 539, 540, 530, 531, and 532, respectively; (iii) the HCDR1, HCDR2, HCDR3, LCDR1, LCDR2, and LCDR3 comprise the amino acid sequences of SEQ ID NO: 542, 543, 540, 533, 534, and 535, respectively; or (iv) the HCDR1, HCDR2, HCDR3, LCDR1, LCDR2, and LCDR3 comprise the amino acid sequences of SEQ ID NO: 544, 545, 546, 536, 534, and 532, respectively; and wherein the Fc region comprises: (a) a L234A, L235A, S267K, and P329A mutation (LALASKPA), numbered according to the EU numbering system; (b) a L234A, L235A, and P329G mutation (LALAPG), numbered according to the EU numbering system; (c) a G237A, D265A, P329A, and S267K mutation (GADAPASK), numbered according to the EU numbering system; (d) a L234A, L235A, and G237A mutation (LALGA), numbered according to the EU numbering system; (e) a D265A, P329A, and S267K mutation (DAPASK), numbered according to the EU numbering system; (f) a G237A, D265A, and P329A mutation (GADAPA), numbered according to the EU numbering system; or (g) a L234A, L235A, and P329A mutation (LALAPA), numbered according to the EU numbering system

In some embodiments, the anti-CD28 antibody molecule described herein comprises: (i) a VH comprising the amino acid sequence of SEQ ID NO: 547 or 548, or a sequence with at least 95% sequence identity to SEQ ID NO: 547 or 548; and/or (ii) a VL comprising the amino acid sequence of SEQ ID NO: 537, or a sequence with at least 95% sequence identity thereto. In some embodiments, the anti-CD28 antibody molecule comprises: (i) a VH comprising the amino acid sequence of SEQ ID NO: 547 or a sequence with at least 95% sequence identity thereto, and a VL comprising the amino acid sequence of SEQ ID NO: 537, or a sequence with at least 95% sequence identity thereto; or (ii) a VH comprising the amino acid sequence of SEQ ID NO: 548 or a sequence with at least 95% sequence identity thereto, and a VL comprising the amino acid sequence of SEQ ID NO: 537, or a sequence with at least 95% sequence identity thereto. In some embodiments, the anti-CD28 antibody molecule is a human antibody, a full length antibody, a bispecific antibody, Fab, F(ab′)2, Fv, or a single chain Fv fragment (scFv). In some embodiments, the antibody molecule comprises a heavy chain constant region selected from IgG1, IgG2, IgG3, and IgG4, and a light chain constant region chosen from the light chain constant regions of kappa or lambda.

In some aspects, the disclosure features an antibody molecule that (i) competes for binding to CD28 with an anti-CD28 antibody molecule described herein; and/or (ii) binds to the same epitope as, substantially the same epitope as, an epitope that overlaps with, or an epitope that substantially overlaps with, the epitope of an anti-CD28 antibody molecule described herein.

In some aspects, the disclosure features an antibody molecule comprising an Fc region that (i) competes for binding to CD28 with an anti-CD28 antibody molecule described herein; and/or (ii) binds to the same epitope as, substantially the same epitope as, an epitope that overlaps with, or an epitope that substantially overlaps with, the epitope of an anti-CD28 antibody molecule described herein, wherein the Fc region is silenced by a combination of amino acid substitutions selected from the group consisting of LALGA (L234A, L235A, and G237A), LALASKPA (L234A, L235A, S267K, and P329A), DAPASK (D265A, P329A, and S267K), GADAPA (G237A, D265A, and P329A), GADAPASK (G237A, D265A, P329A, and S267K), LALAPG (L234A, L235A, and P329G), and LALAPA (L234A, L235A, and P329A), wherein the amino acid residues are numbered according to the EU numbering system.

In some aspects, the disclosure features a multispecific binding molecule comprising: (i) an anti-CD3 binding domain, and (ii) a CD28 antigen binding domain comprising an anti-CD28 antibody molecule described herein. In some embodiments, the anti-CD3 binding domain comprises: (i) a HCDR1, HCDR2, HCDR3, LCDR1, LCDR2, and LCDR3 of an anti-CD3 antibody molecule of Table 27 (for example the anti-CD3 (1), anti-CD3 (2), anti-CD3 (3), or anti-CD3 (4)); (ii) the amino acid sequence of any one of the VH and/or VL region of an anti-CD3 antibody molecule provided in Table 27 (for example the anti-CD3 (1), anti-CD3 (2), anti-CD3 (3), or anti-CD3 (4)), or an amino acid sequence at least 95% identical thereto.

In some aspects, the disclosure features a multispecific binding molecule comprising: (i) an anti-CD3 binding domain, (ii) an anti-CD28 binding domain comprising a heavy chain variable region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3, wherein: (a) the HCDR1, HCDR2, HCDR3, LCDR1, LCDR2, and LCDR3 comprise the amino acid sequences of SEQ ID NOs: 538, 539, 540, 530, 531, and 532, respectively; (b) the HCDR1, HCDR2, HCDR3, LCDR1, LCDR2, and LCDR3 comprise the amino acid sequences of SEQ ID NOs: 541, 539, 540, 530, 531, and 532, respectively; (c) the HCDR1, HCDR2, HCDR3, LCDR1, LCDR2, and LCDR3 comprise the amino acid sequences of SEQ ID NOs: 542, 543, 540, 533, 534, and 535, respectively; or (d) the HCDR1, HCDR2, HCDR3, LCDR1, LCDR2, and LCDR3 comprise the amino acid sequences of SEQ ID NOs: 544, 545, 546, 536, 534, and 532, respectively; and (iii) an Fc region comprising: a L234A, L235A, S267K, and P329A mutation (LALASKPA), numbered according to the EU numbering system; a L234A, L235A, and P329G mutation (LALAPG), numbered according to the EU numbering system; a G237A, D265A, P329A, and S267K mutation (GADAPASK), numbered according to the EU numbering system; a L234A, L235A, and G237A mutation (LALGA), numbered according to the EU numbering system; a D265A, P329A, and S267K mutation (DAPASK), numbered according to the EU numbering system; a G237A, D265A, and P329A mutation (GADAPA), numbered according to the EU numbering system; or a L234A, L235A, and P329A mutation (LALAPA), numbered according to the EU numbering system.

In some aspects, the disclosure features a multispecific binding molecule, comprising a first binding domain and a second binding domain: (i) a first polypeptide comprising from N-terminal to C-terminal: VH of the first binding domain, VL of the first binding domain, VH of the second binding domain, CH1, an Fc region comprising a CH2 and a CH3; and (ii) a second polypeptide comprising from N-terminal to C-terminal: VL of the second binding domain and CL; wherein the Fc region comprises: a L234A, L235A, S267K, and P329A mutation (LALASKPA), numbered according to the EU numbering system; a L234A, L235A, and P329G mutation (LALAPG), numbered according to the EU numbering system; a G237A, D265A, P329A, and S267K mutation (GADAPASK), numbered according to the EU numbering system; a L234A, L235A, and G237A mutation (LALGA), numbered according to the EU numbering system; a D265A, P329A, and S267K mutation (DAPASK), numbered according to the EU numbering system; a G237A, D265A, and P329A mutation (GADAPA), numbered according to the EU numbering system; or a L234A, L235A, and P329A mutation (LALAPA), numbered according to the EU numbering system. In some embodiments, the first binding domain comprises an anti-CD3 binding domain and the second binding domain comprises a costimulatory molecule binding domain. In some embodiments, the first binding domain comprises a costimulatory molecule binding domain and the second binding domain comprises an anti-CD3 binding domain. In some embodiments, the costimulatory molecule binding domain comprises an anti-CD2 binding domain or an anti-CD28 binding domain.

In some aspects, the disclosure features a multispecific binding molecule comprising a first binding domain and a second binding domain: (i) a first polypeptide comprising from N-terminal to C-terminal: VH of the second binding domain, CH1, and an Fc region comprising a CH2 and a CH3, VH of the first binding domain, and VL of the first binding domain; and (ii) a second polypeptide comprising from N-terminal to C-terminal: VL of the second binding domain and CL; wherein the Fc region comprises: a L234A, L235A, S267K, and P329A mutation (LALASKPA), numbered according to the EU numbering system; a L234A, L235A, and P329G mutation (LALAPG), numbered according to the EU numbering system; a G237A, D265A, P329A, and S267K mutation (GADAPASK), numbered according to the EU numbering system; a L234A, L235A, and G237A mutation (LALGA), numbered according to the EU numbering system; a D265A, P329A, and S267K mutation (DAPASK), numbered according to the EU numbering system; a G237A, D265A, and P329A mutation (GADAPA), numbered according to the EU numbering system; or a L234A, L235A, and P329A mutation (LALAPA), numbered according to the EU numbering system.

In some embodiments, the first binding domain comprises an anti-CD3 binding domain and the second binding domain comprises a costimulatory molecule binding domain. In some embodiments, the first binding domain comprises a costimulatory molecule binding domain and the second binding domain comprises an anti-CD3 binding domain. In some embodiments, the costimulatory molecule binding domain comprises an anti-CD2 binding domain or an anti-CD28 binding domain.

In some aspects, the disclosure features a multispecific binding molecule comprising a first binding domain and a second binding domain: (i) a first polypeptide comprising from N-terminal to C-terminal: VH of the second binding domain, CH1, VH of the first binding domain, VL of the first binding domain, an Fc region comprising a CH2 and a CH3; and (ii) a second polypeptide comprising from N-terminal to C-terminal: VL of the second binding domain and CL; wherein the Fc region comprises: a L234A, L235A, S267K, and P329A mutation (LALASKPA), numbered according to the EU numbering system; a L234A, L235A, and P329G mutation (LALAPG), numbered according to the EU numbering system; a G237A, D265A, P329A, and S267K mutation (GADAPASK), numbered according to the EU numbering system; a L234A, L235A, and G237A mutation (LALGA), numbered according to the EU numbering system; a D265A, P329A, and S267K mutation (DAPASK), numbered according to the EU numbering system; a G237A, D265A, and P329A mutation (GADAPA), numbered according to the EU numbering system; or a L234A, L235A, and P329A mutation (LALAPA), numbered according to the EU numbering system. In some embodiments, the first binding domain comprises an anti-CD3 binding domain and the second binding domain comprises a costimulatory molecule binding domain. In some embodiments, the first binding domain comprises a costimulatory molecule binding domain and the second binding domain comprises an anti-CD3 binding domain. In some embodiments, the costimulatory molecule binding domain comprises an anti-CD2 binding domain or an anti-CD28 binding domain.

In some embodiments, the Fc region, e.g., the Fc region of a multispecific binding molecule described herein or to be used in a method described herein, is silenced by a combination of amino acid substitutions selected from the group consisting of LALGA (L234A, L235A, and G237A), LALASKPA (L234A, L235A, S267K, and P329A), DAPASK (D265A, P329A, and S267K), GADAPA (G237A, D265A, and P329A), GADAPASK (G237A, D265A, P329A, and S267K), LALAPG (L234A, L235A, and P329G), and LALAPA (L234A, L235A, and P329A), wherein the amino acid residues are numbered according to the EU numbering system.

In some embodiments, the Fc region, e.g., the Fc region of a multispecific binding molecule described herein or to be used in a method described herein, comprises: a L234A, L235A, S267K, and P329A mutation (LALASKPA), numbered according to the EU numbering system; a L234A, L235A, and P329G mutation (LALAPG), numbered according to the EU numbering system; a G237A, D265A, P329A, and S267K mutation (GADAPASK), numbered according to the EU numbering system; a L234A, L235A, and G237A mutation (LALGA), numbered according to the EU numbering system; a D265A, P329A, and S267K mutation (DAPASK), numbered according to the EU numbering system; a G237A, D265A, and P329A mutation (GADAPA), numbered according to the EU numbering system; or a L234A, L235A, and P329A mutation (LALAPA), numbered according to the EU numbering system.

In some embodiments, a multispecific binding molecule comprises a heavy chain comprising the amino acid sequence of any of SEQ ID NOs: 794, 795, 798, 800, or 815-816 or an amino acid sequence having at least 95% sequence identity thereto; and/or a light chain comprising the amino acid sequence of any of SEQ ID NOs: 673, 796, 797, 799, or 801, or an amino acid sequence having at least 95% sequence identity thereto. In some embodiments, the multispecific binding molecule comprises a heavy chain comprising the amino acid sequence of SEQ ID NO: 794 or an amino acid sequence having at least 95% sequence identity thereto, and a light chain comprising the amino acid sequence of SEQ ID NO: 796, or an amino acid sequence having at least 95% sequence identity thereto. In some embodiments, the multispecific binding molecule comprises a heavy chain comprising the amino acid sequence of SEQ ID NO: 794 or an amino acid sequence having at least 95% sequence identity thereto, and a light chain comprising the amino acid sequence of SEQ ID NO: 797, or an amino acid sequence having at least 95% sequence identity thereto. In some embodiments, the multispecific binding molecule comprises a heavy chain comprising the amino acid sequence of SEQ ID NO: 795 or an amino acid sequence having at least 95% sequence identity thereto, and a light chain comprising the amino acid sequence of SEQ ID NO: 796, or an amino acid sequence having at least 95% sequence identity thereto. In some embodiments, the 20) multispecific binding molecule comprises a heavy chain comprising the amino acid sequence of SEQ ID NO: 795 or an amino acid sequence having at least 95% sequence identity thereto, and a light chain comprising the amino acid sequence of SEQ ID NO: 797, or an amino acid sequence having at least 95% sequence identity thereto. In some embodiments, the multispecific binding molecule comprises a heavy chain comprising the amino acid sequence of SEQ ID NO: 798 or an amino acid sequence having at least 95% sequence identity thereto, and a light chain comprising the amino acid sequence of SEQ ID NO: 799, or an amino acid sequence having at least 95% sequence identity thereto. In some embodiments, the multispecific binding molecule comprises a heavy chain comprising the amino acid sequence of SEQ ID NO: 815 or an amino acid sequence having at least 95% sequence identity thereto, and a light chain comprising the amino acid sequence of SEQ ID NO: 799, or an amino acid sequence having at least 95% sequence identity thereto. In some embodiments, the multispecific binding molecule comprises a heavy chain comprising the amino acid sequence of SEQ ID NO: 800 or an amino acid sequence having at least 95% sequence identity thereto, and a light chain comprising the amino acid sequence of SEQ ID NO: 801, or an amino acid sequence having at least 95% sequence identity thereto. In some embodiments, the multispecific binding molecule comprises a heavy chain comprising the amino acid sequence of SEQ ID NO: 816 or an amino acid sequence having at least 95% sequence identity thereto, and a light chain comprising the amino acid sequence of SEQ ID NO: 673, or an amino acid sequence having at least 95% sequence identity thereto. In some embodiments, the multispecific binding molecule comprises a heavy chain comprising the amino acid sequence of SEQ ID NO: 817 or an amino acid sequence having at least 95% sequence identity thereto, and a light chain comprising the amino acid sequence of SEQ ID NO: 673, or an amino acid sequence having at least 95% sequence identity thereto.

In some aspects, the disclosure features a method of activating cells (e.g., immune effector cells, e.g., T cells), comprising contacting (for example, binding) a population of cells (for example, T cells, for example, T cells isolated from a frozen or fresh leukapheresis product) with a multispecific binding molecule described herein.

In some aspects, the disclosure features a method of transducing cells (e.g., immune effector cells, e.g., T cells), comprising contacting (for example, binding) a population of cells (for example, T cells, for example, T cells isolated from a frozen or fresh leukapheresis product) with (i) a multispecific binding molecule described herein and (ii) a nucleic acid molecule, e.g., a nucleic acid molecule encoding a CAR.

Those skilled in the art will recognize or be able to ascertain using no more than routine experimentation, many equivalents to the specific embodiments of the invention described herein. Such equivalents are intended to be encompassed by the following enumerated embodiments.

### ENUMERATED EMBODIMENTS

1. A method of making a population of cells (for example, T cells) that express a chimeric antigen receptor (CAR), the method comprising:
(i) contacting (for example, binding) a population of cells (for example, T cells, for example, T cells isolated from a frozen or fresh leukapheresis product) with a multispecific binding molecule comprising (A) an anti-CD3 binding domain, (B) a costimulatory molecule binding domain (e.g., an anti-CD2 binding domain or an anti-CD28 binding domain), and (C) an Fc region comprising:
   a L234A, L235A, S267K, and P329A mutation (LALASKPA), numbered according to the EU numbering system;
   a L234A, L235A, and P329G mutation (LALAPG), numbered according to the EU numbering system;
   a G237A, D265A, P329A, and S267K mutation (GADAPASK), numbered according to the EU numbering system;
   a L234A, L235A, and G237A mutation (LALGA), numbered according to the EU numbering system;
   a D265A, P329A, and S267K mutation (DAPASK), numbered according to the EU numbering system;
   a G237A, D265A, and P329A mutation (GADAPA), numbered according to the EU numbering system; or
   a L234A, L235A, and P329A mutation (LALAPA), numbered according to the EU numbering system;
(ii) contacting the population of cells (for example, T cells) with a nucleic acid molecule (for example, a DNA or RNA molecule) encoding the CAR, thereby providing a population of cells (for example, T cells) comprising the nucleic acid molecule, and
(iii) harvesting the population of cells (for example, T cells) for storage (for example, reformulating the population of cells in cryopreservation media) or administration, wherein:
(a) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and
   step (iii) is performed no later than 30 (for example, 26) hours after the beginning of step (i), for example, no later than 22, 23, 24, 25, 26, 27, 28, 29, or 30 hours after the beginning of step (i), for example, no later than 24 hours after the beginning of step (i),
(b) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and
   step (iii) is performed no later than 30 hours after the beginning of step (ii), for example, no later than 22, 23, 24, 25, 26, 27, 28, 29, or 30 hours after the beginning of step (ii), or
(c) the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i),
optionally wherein the nucleic acid molecule in step (ii) is on a viral vector, optionally wherein the nucleic acid molecule in step (ii) is an RNA molecule on a viral vector, optionally wherein step (ii) comprises transducing the population of cells (for example, T cells) with a viral vector comprising a nucleic acid molecule encoding the CAR.

2. The method of embodiment 1, wherein:
(i) the anti-CD3 binding domain, e.g., an anti-CD3 scFv, is situated N-terminal of the costimulatory molecule binding domain, e.g., an anti-CD2 Fab or an anti-CD28 Fab; or
(ii) the anti-CD3 binding domain, e.g., an anti-CD3 scFv, is situated C-terminal of the costimulatory molecule binding domain, e.g., an anti-CD2 Fab or an anti-CD28 Fab.

3. The method of embodiment 1 or 2, wherein the Fc region comprises a CH2.

4. The method of any one of embodiments 1-3, wherein the Fc region comprises a CH3.

5. The method of any one of embodiments 1-4, wherein the anti-CD3 binding domain is situated C-terminal of the Fc region.

6. The method of any one of embodiments 1-4, wherein the anti-CD3 binding domain is situated N-terminal of the Fc region.

7. The method of any one of embodiments 1-6, wherein the Fc region is situated between the anti-CD3 binding domain and the costimulatory molecule binding domain.

8. The method of any one of embodiments 1-4 or 6, wherein the multispecific binding molecule comprises:
(i) a first polypeptide comprising from N-terminal to C-terminal: VH of the anti-CD3 binding domain, VL of the anti-CD3 binding domain, VH of the costimulatory molecule binding domain, CH1, CH2, and CH3; and
(ii) a second polypeptide comprising from N-terminal to C-terminal: VL of the costimulatory molecule binding domain and CL.

9. The method of any one of embodiments 1-5 or 7, wherein the multispecific binding molecule comprises:
(i) a first polypeptide comprising from N-terminal to C-terminal: VH of the costimulatory molecule binding domain, CH1, CH2, CH3, VH of the anti-CD3 binding domain, and VL of the anti-CD3 binding domain; and
(ii) a second polypeptide comprising from N-terminal to C-terminal: VL of the costimulatory molecule binding domain and CL.

10. The method of any one of embodiments 1-4 or 6, wherein the multispecific binding molecule comprises:
(i) a first polypeptide comprising from N-terminal to C-terminal: VH of the costimulatory molecule binding domain, CH1, VH of the anti-CD3 binding domain, VL of the anti-CD3 binding domain, CH2, and CH3; and
(ii) a second polypeptide comprising from N-terminal to C-terminal: VL of the costimulatory molecule binding domain and CL.

11. The method of any one of embodiments 1-10, wherein the anti-CD3 binding domain comprises an scFv and the costimulatory molecule binding domain is part of a Fab fragment.

12. The method of any one of embodiments 1-11, wherein the anti-CD3 binding domain comprises:
(i) a variable heavy chain region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3 of an anti-CD3 antibody molecule of Table 27 (for example the anti-CD3 (1), anti-CD3 (2), anti-CD3 (3), or anti-CD3 (4)); and/or
(ii) the amino acid sequence of any VH and/or VL region of an anti-CD3 antibody molecule provided in Table 27 (for example the anti-CD3 (1), anti-CD3 (2), anti-CD3 (3), or anti-CD3 (4)), or an amino acid sequence at least 95% identical thereto.

13. The method of any one of embodiments 1-12, wherein the costimulatory molecule binding domain is an anti-CD2 binding domain, optionally wherein the anti-CD2 binding domain comprises:
(i) a VH comprising a HCDR1, a HCDR2, and a HCDR3, and a VL comprising a LCDR1, a LCDR2, and a LCDR3 of an anti-CD2 antibody molecule of Table 27 (for example the anti-CD2 (1)); and/or
(ii) the amino acid sequence of any VH and/or VL region of an anti-CD2 antibody molecule provided in Table 27 (for example the anti-CD2 (1)), or an amino acid sequence at least 95% identical thereto.

14. The method of any one of embodiments 1-13, wherein the costimulatory molecule binding domain is an anti-CD28 binding domain, optionally wherein the anti-CD28 binding domain comprises:
(i) a VH comprising a HCDR1, a HCDR2, and a HCDR3, and a VL comprising a LCDR1, a LCDR2, and a LCDR3 of an anti-CD28 antibody molecule of Table 27 (for example the anti-CD28 (1) or anti-CD28 (2)); and/or
(ii) the amino acid sequence of any VH and/or VL region of an anti-CD28 antibody molecule provided in Table 27 (for example the anti-CD28 (1) or anti-CD28 (2)), or an amino acid sequence at least 95% identical thereto.

15. The method of any one of embodiments 1-14, wherein the anti-CD3 binding domain comprises:
(i) an scFv;
(ii) a VH linked to a VL by a peptide linker, e.g., a glycine-serine linker, e.g., a (G₄S)₄ linker; or
(iii) a VH and a VL, wherein the VH is N-terminal of the VL.

16. The method of any one of embodiments 1-15, wherein the costimulatory molecule binding domain is part of a Fab fragment, e.g., a Fab fragment that is part of a polypeptide sequence that comprises the Fc region.

17. The method of any one of embodiments 1-16, wherein the anti-CD3 binding domain is situated N-terminal of the costimulatory molecule binding domain, optionally wherein the anti-CD3 binding domain is linked to the costimulatory molecule binding domain by a peptide linker, e.g., a glycine-serine linker, e.g., a (G₄S)₄ linker.

18. The method of any one of embodiments 1-7 or 9-17, wherein the anti-CD3 binding domain is situated C-terminal of the costimulatory molecule binding domain.

19. The method of embodiment 18, wherein:
(i) the Fc region is situated between the anti-CD3 binding domain and the costimulatory molecule binding domain; and/or
(ii) the multispecific binding molecule comprises one or both of a CH2 and a CH3, optionally wherein the anti-CD3 binding domain is linked to the CH3 by a peptide linker, e.g., a glycine-serine linker, e.g., a (G₄S)₄ linker.

20. The method of embodiment 18, wherein:
(i) the multispecific binding molecule comprises a CH2, and the anti-CD3 binding domain is situated N-terminal of the CH2;
(ii) the anti-CD3 binding domain is linked to a CH1 by a peptide linker, e.g., a glycine-serine linker, e.g., a (G₄S)₂ linker; and/or
(iii) the anti-CD3 binding domain is linked to a CH2 by a peptide linker, e.g., a glycine-serine linker, e.g., a (G₄S)₄ linker.

21. The method of any one of embodiments 1-20, wherein step (i) increases the percentage of CAR-expressing cells in the population of cells from step (iii), for example, the population of cells from step (iii) shows a higher percentage of CAR-expressing cells (for example, at least 10, 20, 30, 40, 50, or 60% higher), compared with cells made by an otherwise similar method without step (i).

22. The method of any one of embodiments 1-21, wherein:
(a) the percentage of naïve cells, for example, naïve T cells, for example, CD45RA+CD45RO− CCR7+ T cells, in the population of cells from step (iii) is the same as or differs by no more than 5 or 10% from the percentage of naïve cells, for example, naïve T cells, for example, CD45RA+CD45RO− CCR7+ cells, in the population of cells at the beginning of step (i);
(b) the percentage of naïve cells, for example, naïve T cells, for example, CD45RA+CD45RO− CCR7+ T cells, in the population of cells from step (iii) is increased by, for example, at least 1.2, 1.4, 1.6, 1.8, 2.0, 2.2, 2.4, 2.6, 2.8, or 3-fold, as compared to the percentage of naïve cells, for example, naïve T cells, for example, CD45RA+CD45RO− CCR7+ cells, in the population of cells at the beginning of step (i);
(c) the percentage of CAR-expressing naïve T cells, for example, CAR-expressing CD45RA+CD45RO− CCR7+ T cells in the population of cells increases during the duration of step (ii), for example, increases by, for example, at least 30, 35, 40, 45, 50, 55, or 60%, between 18-24 hours after the beginning of step (ii); or
(d) the percentage of naïve cells, for example, naïve T cells, for example, CD45RA+CD45RO− CCR7+ T cells, in the population of cells from step (iii) does not decrease, or decreases by no more than 5 or 10%, as compared to the percentage of naïve cells, for example, naïve T cells, for example, CD45RA+CD45RO− CCR7+ cells, in the population of cells at the beginning of step (i).

23. The method of any one of embodiments 1-22, wherein:
(a) the population of cells from step (iii) shows a higher percentage of naïve cells, for example, naïve T cells, for example, CD45RA+CD45RO− CCR7+ T cells (for example, at least 10, 20, 30, or 40% higher), compared with cells made by an otherwise similar method in which step (iii) is performed more than 26 hours after the beginning of step (i), for example, more than 5, 6, 7, 8, 9, 10, 11, or 12 days after the beginning of step (i);
(b) the percentage of naïve cells, for example, naïve T cells, for example, CD45RA+CD45RO− CCR7+ T cells, in the population of cells from step (iii) is higher (for example, at least 1.2, 1.4, 1.6, 1.8, 2.0, 2.2, 2.4, 2.6, 2.8, or 3-fold higher) than the percentage of naïve cells, for example, naïve T cells, for example, CD45RA+CD45RO− CCR7+ T cells, in cells made by an otherwise similar method in which step (iii) is performed more than 26 hours after the beginning of step (i), for example, more than 5, 6, 7, 8, 9, 10, 11, or 12 days after the beginning of step (i);
(c) the percentage of CAR-expressing naïve T cells, for example, CAR-expressing CD45RA+CD45RO− CCR7+ T cells, in the population of cells from step (iii) is higher (for example, at least 4, 6, 8, 10, or 12-fold higher) than the percentage of CAR-expressing naïve T cells, for example, CAR-expressing CD45RA+CD45RO− CCR7+ T cells, in cells made by an otherwise similar method in which step (iii) is performed more than 26 hours after the beginning of step (i), for example, more than 5, 6, 7, 8, 9, 10, 11, or 12 days after the beginning of step (i);
(d) the population of cells from step (iii) shows a higher percentage of naïve cells, for example, naïve T cells, for example, CD45RA+CD45RO− CCR7+ T cells (for example, at least 10, 20, 30, or 40% higher), compared with cells made by an otherwise similar method which further comprises, after step (ii) and prior to step (iii), expanding the population of cells (for example, T cells) in vitro for more than 3 days, for example, for 5, 6, 7, 8 or 9 days;
(e) the percentage of naïve cells, for example, naïve T cells, for example, CD45RA+CD45RO− CCR7+ T cells, in the population of cells from step (iii) is higher (for example, at least 1.2, 1.4, 1.6, 1.8, 2.0, 2.2, 2.4, 2.6, 2.8, or 3-fold higher) than the percentage of naïve cells, for example, naïve T cells, for example, CD45RA+CD45RO− CCR7+ T cells, in cells made by an otherwise similar method which further comprises, after step (ii) and prior to step (iii), expanding the population of cells (for example, T cells) in vitro for more than 3 days, for example, for 5, 6, 7, 8 or 9 days; or
(f) the percentage of CAR-expressing naïve T cells, for example, CAR-expressing CD45RA+CD45RO− CCR7+ T cells, in the population of cells from step (iii) is higher (for example, at least 4, 6, 8, 10, or 12-fold higher) than the percentage of CAR-expressing naïve T cells, for example, CAR-expressing CD45RA+CD45RO− CCR7+ T cells, in cells made by an otherwise similar method which further comprises, after step (ii) and prior to step (iii), expanding the population of cells (for example, T cells) in vitro for more than 3 days, for example, for 5, 6, 7, 8 or 9 days.

24. The method of any one of embodiments 1-23, wherein:
(a) the percentage of central memory cells, for example, central memory T cells, for example, CD95+ central memory T cells, in the population of cells from step (iii) is the same as or differs by no more than 5 or 10% from the percentage of central memory cells, for example, central memory T cells, for example, CD95+ central memory T cells, in the population of cells at the beginning of step (i);
(b) the percentage of central memory cells, for example, central memory T cells, for example, CCR7+CD45RO+ T cells, in the population of cells from step (iii) is reduced by at least 20, 25, 30, 35, 40, 45, or 50%, as compared to the percentage of central memory cells, for example, central memory T cells, for example, CCR7+CD45RO+ T cells, in the population of cells at the beginning of step (i);
(c) the percentage of CAR-expressing central memory T cells, for example, CAR-expressing CCR7+CD45RO+ cells, decreases during the duration of step (ii), for example, decreases by, for example, at least 8, 10, 12, 14, 16, 18, or 20%, between 18-24 hours after the beginning of step (ii); or
(d) the percentage of central memory cells, for example, central memory T cells, for example, CCR7+CD45RO+ T cells, in the population of cells from step (iii) does not increase, or increases by no more than 5 or 10%, as compared to the percentage of central memory cells, for example, central memory T cells, for example, CCR7+CD45RO+ T cells, in the population of cells at the beginning of step (i).

25. The method of any one of embodiments 1-24, wherein:
(a) the population of cells from step (iii) shows a lower percentage of central memory cells, for example, central memory T cells, for example, CD95+ central memory T cells (for example, at least 10, 20, 30, or 40% lower), compared with cells made by an otherwise similar method in which step (iii) is performed more than 26 hours after the beginning of step (i), for example, more than 5, 6, 7, 8, 9, 10, 11, or 12 days after the beginning of step (i);
(b) the percentage of central memory cells, for example, central memory T cells, for example, CCR7+CD45RO+ T cells in the population of cells from step (iii) is lower (for example, at least 20, 30, 40, or 50% lower) than the percentage of central memory cells, for example, central memory T cells, for example, CCR7+CD45RO+ T cells, in cells made by an otherwise similar method in which step (iii) is performed more than 26 hours after the beginning of step (i), for example, more than 5, 6, 7, 8, 9, 10, 11, or 12 days after the beginning of step (i);
(c) the percentage of CAR-expressing central memory T cells, for example, CAR-expressing CCR7+CD45RO+ T cells in the population of cells from step (iii) is lower (for example, at least 10, 20, 30, or 40% lower) than the percentage of CAR-expressing central memory T cells, for example, CAR-expressing CCR7+CD45RO+ T cells, in cells made by an otherwise similar method in which step (iii) is performed more than 26 hours after the beginning of step (i), for example, more than 5, 6, 7, 8, 9, 10, 11, or 12 days after the beginning of step (i);
(d) the population of cells from step (iii) shows a lower percentage of central memory cells, for example, central memory T cells, for example, CD95+ central memory T cells (for example, at least 10, 20, 30, or 40% lower), compared with cells made by an otherwise similar method which further comprises, after step (ii) and prior to step (iii), expanding the population of cells (for example, T cells) in vitro for more than 3 days, for example, for 5, 6, 7, 8 or 9 days;
(e) the percentage of central memory cells, for example, central memory T cells, for example, CCR7+CD45RO+ T cells in the population of cells from step (iii) is lower (for example, at least 20, 30, 40, or 50% lower) than the percentage of central memory cells, for example, central memory T cells, for example, CCR7+CD45RO+ T cells, in cells made by an otherwise similar method which further comprises, after step (ii) and prior to step (iii), expanding the population of cells (for example, T cells) in vitro for more than 3 days, for example, for 5, 6, 7, 8 or 9 days; or
(f) the percentage of CAR-expressing central memory T cells, for example, CAR-expressing CCR7+CD45RO+ T cells in the population of cells from step (iii) is lower (for example, at least 10, 20, 30, or 40% lower) than the percentage of CAR-expressing central memory T cells, for example, CAR-expressing CCR7+CD45RO+ T cells, in cells made by an otherwise similar method which further comprises, after step (ii) and prior to step (iii), expanding the population of cells (for example, T cells) in vitro for more than 3 days, for example, for 5, 6, 7, 8 or 9 days.

26. The method of any one of embodiments 1-25, wherein:
(a) the percentage of stem memory T cells, for example, CD45RA+CD95+IL-2 receptor β+CCR7+CD62L+ T cells, in the population of cells from step (iii) is increased, as compared to the percentage of stem memory T cells, for example, CD45RA+CD95+IL-2 receptor β+CCR7+CD62L+ T cells, in the population of cells at the beginning of step (i);
(b) the percentage of CAR-expressing stem memory T cells, for example, CAR-expressing CD45RA+CD95+IL-2 receptor β+CCR7+CD62L+ T cells, in the population of cells from step (iii) is increased, as compared to the percentage of CAR-expressing stem memory T cells, for example, CAR-expressing CD45RA+CD95+IL-2 receptor β+CCR7+CD62L+ T cells, in the population of cells at the beginning of step (i);
(c) the percentage of stem memory T cells, for example, CD45RA+CD95+IL-2 receptor β+CCR7+CD62L+ T cells, in the population of cells from step (iii) is higher than the percentage of stem memory T cells, for example, CD45RA+CD95+IL-2 receptor β+CCR7+CD62L+ T cells, in cells made by an otherwise similar method in which step (iii) is performed more than 26 hours after the beginning of step (i), for example, more than 5, 6, 7, 8, 9, 10, 11, or 12 days after the beginning of step (i); or
(d) the percentage of CAR-expressing stem memory T cells, for example, CAR-expressing CD45RA+CD95+IL-2 receptor β+CCR7+CD62L+ T cells, in the population of cells from step (iii) is higher than the percentage of CAR-expressing stem memory T cells, for example, CAR-expressing CD45RA+CD95+IL-2 receptor β+CCR7+CD62L+ T cells, in cells made by an otherwise similar method in which step (iii) is performed more than 26 hours after the beginning of step (i), for example, more than 5, 6, 7, 8, 9, 10, 11, or 12 days after the beginning of step (i);
(e) the percentage of stem memory T cells, for example, CD45RA+CD95+IL-2 receptor β+CCR7+CD62L+ T cells, in the population of cells from step (iii) is higher than the percentage of stem memory T cells, for example, CD45RA+CD95+IL-2 receptor β+CCR7+CD62L+ T cells, in cells made by an otherwise similar method which further comprises, after step (ii) and prior to step (iii), expanding the population of cells (for example, T cells) in vitro for more than 3 days, for example, for 5, 6, 7, 8 or 9 days; or
(f) the percentage of CAR-expressing stem memory T cells, for example, CAR-expressing CD45RA+CD95+IL-2 receptor β+CCR7+CD62L+ T cells, in the population of cells from step (iii) is higher than the percentage of CAR-expressing stem memory T cells, for example, CAR-expressing CD45RA+CD95+IL-2 receptor β+CCR7+CD62L+ T cells, in cells made by an otherwise similar method which further comprises, after step (ii) and prior to step (iii), expanding the population of cells (for example, T cells) in vitro for more than 3 days, for example, for 5, 6, 7, 8 or 9 days.

27. The method of any one of embodiments 1-26, wherein:
(a) the median GeneSetScore (Up TEM vs. Down TSCM) of the population of cells from step (iii) is about the same as or differs by no more than (for example, increased by no more than) about 25, 50, 75, 100, or 125% from the median GeneSetScore (Up TEM vs. Down TSCM) of the population of cells at the beginning of step (i);
(b) the median GeneSetScore (Up TEM vs. Down TSCM) of the population of cells from step (iii) is lower (for example, at least about 100, 150, 200, 250, or 300% lower) than the median GeneSetScore (Up TEM vs. Down TSCM) of:
   cells made by an otherwise similar method in which step (iii) is performed more than 26 hours after the beginning of step (i), for example, more than 5, 6, 7, 8, 9, 10, 11, or 12 days after the beginning of step (i), or
   cells made by an otherwise similar method which further comprises, after step (ii) and prior to step (iii), expanding the population of cells (for example, T cells) in vitro for more than 3 days, for example, for 5, 6, 7, 8 or 9 days;
(c) the median GeneSetScore (Up Treg vs. Down Teff) of the population of cells from step (iii) is about the same as or differs by no more than (for example, increased by no more than) about 25, 50, 100, 150, or 200% from the median GeneSetScore (Up Treg vs. Down Teff) of the population of cells at the beginning of step (i);
(d) the median GeneSetScore (Up Treg vs. Down Teff) of the population of cells from step (iii) is lower (for example, at least about 50, 100, 125, 150, or 175% lower) than the median GeneSetScore (Up Treg vs. Down Teff) of:
   cells made by an otherwise similar method in which step (iii) is performed more than 26 hours after the beginning of step (i), for example, more than 5, 6, 7, 8, 9, 10, 11, or 12 days after the beginning of step (i), or
   cells made by an otherwise similar method which further comprises, after step (ii) and prior to step (iii), expanding the population of cells (for example, T cells) in vitro for more than 3 days, for example, for 5, 6, 7, 8 or 9 days;
(e) the median GeneSetScore (Down stemness) of the population of cells from step (iii) is about the same as or differs by no more than (for example, increased by no more than) about 25, 50, 100, 150, 200, or 250% from the median GeneSetScore (Down stemness) of the population of cells at the beginning of step (i);
(f) the median GeneSetScore (Down stemness) of the population of cells from step (iii) is lower (for example, at least about 50, 100, or 125% lower) than the median GeneSetScore (Down stemness) of:
   cells made by an otherwise similar method in which step (iii) is performed more than 26 hours after the beginning of step (i), for example, more than 5, 6, 7, 8, 9, 10, 11, or 12 days after the beginning of step (i), or
   cells made by an otherwise similar method which further comprises, after step (ii) and prior to step (iii), expanding the population of cells (for example, T cells) in vitro for more than 3 days, for example, for 5, 6, 7, 8 or 9 days;
(g) the median GeneSetScore (Up hypoxia) of the population of cells from step (iii) is about the same as or differs by no more than (for example, increased by no more than) about 125, 150, 175, or 200% from the median GeneSetScore (Up hypoxia) of the population of cells at the beginning of step (i);
(h) the median GeneSetScore (Up hypoxia) of the population of cells from step (iii) is lower (for example, at least about 40, 50, 60, 70, or 80% lower) than the median GeneSetScore (Up hypoxia) of:
   cells made by an otherwise similar method in which step (iii) is performed more than 26 hours after the beginning of step (i), for example, more than 5, 6, 7, 8, 9, 10, 11, or 12 days after the beginning of step (i), or
   cells made by an otherwise similar method which further comprises, after step (ii) and prior to step (iii), expanding the population of cells (for example, T cells) in vitro for more than 3 days, for example, for 5, 6, 7, 8 or 9 days;
(j) the median GeneSetScore (Up autophagy) of the population of cells from step (iii) is about the same as or differs by no more than (for example, increased by no more than) about 180, 190, 200, or 210% from the median GeneSetScore (Up autophagy) of the population of cells at the beginning of step (i); or
(k) the median GeneSetScore (Up autophagy) of the population of cells from step (iii) is lower (for example, at least 20, 30, or 40% lower) than the median GeneSetScore (Up autophagy) of:
   cells made by an otherwise similar method in which step (iii) is performed more than 26 hours after the beginning of step (i), for example, more than 5, 6, 7, 8, 9, 10, 11, or 12 days after the beginning of step (i), or
   cells made by an otherwise similar method which further comprises, after step (ii) and prior to step (iii), expanding the population of cells (for example, T cells) in vitro for more than 3 days, for example, for 5, 6, 7, 8 or 9 days.

28. The method of any one of embodiments 1-27, wherein the population of cells from step (iii), after being incubated with a cell expressing an antigen recognized by the CAR, secretes IL-2 at a higher level (for example, at least 2, 4, 6, 8, 10, 12, or 14-fold higher) than cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days, for example, as assessed using methods described in Example 8 with respect to FIGS. 29C-29D.

29. The method of any one of embodiments 1-28, wherein the population of cells from step (iii), after being administered in vivo, persists longer or expands at a higher level (for example, as assessed using methods described in Example 1 with respect to FIG. 4C), compared with cells made by an otherwise similar method in which step (iii) is performed more than 26 hours after the beginning of step (i), for example, more than 5, 6, 7, 8, 9, 10, 11, or 12 days after the beginning of step (i), or compared with cells made by an otherwise similar method which further comprises, after step (ii) and prior to step (iii), expanding the population of cells (for example, T cells) in vitro for more than 3 days, for example, for 5, 6, 7, 8 or 9 days.

30. The method of any one of embodiments 1-29, wherein the population of cells from step (iii), after being administered in vivo, shows a stronger anti-tumor activity (for example, a stronger anti-tumor activity at a low dose, for example, a dose no more than 0.15×10⁶, 0.2×10⁶, 0.25×10⁶, or 0.3×10⁶ viable CAR-expressing cells) than cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days.

31. The method of any one of embodiments 1-30, the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i), optionally wherein the number of living cells in the population of cells from step (iii) decreases from the number of living cells in the population of cells at the beginning of step (i).

32. The method of any one of embodiments 1-31, wherein the population of cells from step (iii) are not expanded, or expanded by less than 2 hours, for example, less than 1 or 1.5 hours, compared to the population of cells at the beginning of step (i).

33. The method of any one of embodiments 1-32, wherein steps (i) and/or (ii) are performed in cell media (for example, serum-free media) comprising IL-2, IL-15 (for example, hetIL-15 (IL15/sIL-15Ra)), IL-7, IL-21, IL-6 (for example, IL-6/sIL-6Ra), a LSD1 inhibitor, a MALT1 inhibitor, or a combination thereof.

34. The method of any one of embodiments 1-33, wherein steps (i) and/or (ii) are performed in serum-free cell media comprising a serum replacement.

35. The method of embodiment 30, wherein the serum replacement is CTS™ Immune Cell Serum Replacement (ICSR).

36. The method of any one of embodiments 1-35, further comprising prior to step (i):
(iv) (optionally) receiving a fresh leukapheresis product (or an alternative source of hematopoietic tissue such as a fresh whole blood product, a fresh bone marrow product, or a fresh tumor or organ biopsy or removal (for example, a fresh product from thymectomy)) from an entity, for example, a laboratory, hospital, or healthcare provider, and
(v) isolating the population of cells (for example, T cells, for example, CD8+ and/or CD4+ T cells) contacted in step (i) from a fresh leukapheresis product (or an alternative source of hematopoietic tissue such as a fresh whole blood product, a fresh bone marrow product, or a fresh tumor or organ biopsy or removal (for example, a fresh product from thymectomy)), optionally wherein:
step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v), or the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

37. The method of any one of embodiments 1-36, further comprising prior to step (i): receiving cryopreserved T cells isolated from a leukapheresis product (or an alternative source of hematopoietic tissue such as cryopreserved T cells isolated from whole blood, bone marrow, or tumor or organ biopsy or removal (for example, thymectomy)) from an entity, for example, a laboratory, hospital, or healthcare provider.

38. The method of any one of embodiments 1-36, further comprising prior to step (i):
(iv) (optionally) receiving a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)) from an entity, for example, a laboratory, hospital, or healthcare provider, and
(v) isolating the population of cells (for example, T cells, for example, CD8+ and/or CD4+ T cells) contacted in step (i) from a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)), optionally wherein:
step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v), or
the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

39. The method of any one of embodiments 1-38, further comprising step (vi):
culturing a portion of the population of cells from step (iii) for at least 2, 2.5, 3, 3.5, 4, 4.5, 5, 5.5, 6, 6.5, or 7 days, for example, at least 2 days and no more than 7 days, and measuring CAR expression level in the portion (for example, measuring the percentage of viable, CAR-expressing cells in the portion), optionally wherein:
step (iii) comprises harvesting and freezing the population of cells (for example, T cells) and step (vi) comprises thawing a portion of the population of cells from step (iii), culturing the portion for at least 2, 2.5, 3, 3.5, 4, 4.5, 5, 5.5, 6, 6.5, or 7 days, for example, at least 2 days and no more than 7 days, and measuring CAR expression level in the portion (for example, measuring the percentage of viable, CAR-expressing cells in the portion).

40. The method of any one of embodiments 1-39, wherein step (ii) further comprises adding F108 during transduction and/or contacting the population of cells (for example, T cells) with an shRNA that targets Tet2.

41. The method of any one of embodiments 1-40, wherein the population of cells at the beginning of step (i) or step (1) has been enriched for IL6R-expressing cells (for example, cells that are positive for IL6Rα and/or IL6Rβ).

42. The method of any one of embodiments 1-41, wherein the population of cells at the beginning of step (i) or step (1) comprises no less than 50, 60, or 70% of IL6R-expressing cells (for example, cells that are positive for IL6Rα and/or IL6Rβ).

43. The method of any one of embodiments 1-42, wherein steps (i) and (ii) or steps (1) and (2) are performed in cell media comprising IL-15 (for example, hetIL-15 (IL15/sIL-15Ra)).

44. The method of embodiment 43, wherein IL-15 increases the ability of the population of cells to expand, for example, 10, 15, 20, or 25 days later.

45. The method of embodiment 43, wherein IL-15 increases the percentage of IL6Rβ-expressing cells in the population of cells.

46. The method of any one of embodiments 1-45, wherein the CAR comprises an antigen binding domain, a transmembrane domain, and an intracellular signaling domain.

47. The method of embodiment 46, wherein the antigen binding domain binds to an antigen chosen from: CD19, CD20, CD22, BCMA, mesothelin, EGFRVIII, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, Lewis Y, CD24, PDGFR-beta, SSEA-4, folate receptor alpha, ERBBs (for example, ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRα4, or a peptide of any of these antigens presented on MHC.

48. The method of embodiment 46 or 47, wherein the antigen binding domain comprises a CDR, VH, VL, scFv or CAR sequence disclosed herein, optionally wherein:
(a) the antigen binding domain binds to BCMA and comprises a CDR, VH, VL, scFv or CAR sequence disclosed in Tables 3-15, or a sequence having at least 80%, 85%, 90%, 95%, or 99% identity thereto;
(b) the antigen binding domain binds to CD19 and comprises a CDR, VH, VL, scFv or CAR sequence disclosed in Table 2, or a sequence having at least 80%, 85%, 90%, 95%, or 99% identity thereto;
(c) the antigen binding domain binds to CD20 and comprises a CDR, VH, VL, scFv or CAR sequence disclosed herein, or a sequence having at least 80%, 85%, 90%, 95%, or 99% identity thereto; or
(d) the antigen binding domain binds to CD22 and comprises a CDR, VH, VL, scFv or CAR sequence disclosed herein, or a sequence having at least 80%, 85%, 90%, 95%, or 99% identity thereto.

49. The method of any one of embodiments 46-48, wherein the antigen binding domain comprises a VH and a VL, wherein the VH and VL are connected by a linker, optionally wherein the linker comprises the amino acid sequence of SEQ ID NO: 63 or 104.

50. The method of any one of embodiments 46-49, wherein:
(a) the transmembrane domain comprises a transmembrane domain of a protein chosen from the alpha, beta or zeta chain of T-cell receptor, CD28, CD3 epsilon, CD45, CD4, CD5, CD8, CD9, CD16, CD22, CD33, CD37, CD64, CD80, CD86, CD134, CD137 and CD154,
(b) the transmembrane domain comprises a transmembrane domain of CD8,
(c) the transmembrane domain comprises the amino acid sequence of SEQ ID NO: 6, or an amino acid sequence having at least about 85%, 90%, 95%, or 99% sequence identity thereof, or
(d) the nucleic acid molecule comprises a nucleic acid sequence encoding the transmembrane domain, wherein the nucleic acid sequence comprises the nucleic acid sequence of SEQ ID NO: 17, or a nucleic acid sequence having at least about 85%, 90%, 95%, or 99% sequence identity thereof.

51. The method of any one of embodiments 46-50, wherein the antigen binding domain is connected to the transmembrane domain by a hinge region, optionally wherein:
(a) the hinge region comprises the amino acid sequence of SEQ ID NO: 2, 3, or 4, or an amino acid sequence having at least about 85%, 90%, 95%, or 99% sequence identity thereof, or
(b) the nucleic acid molecule comprises a nucleic acid sequence encoding the hinge region, wherein the nucleic acid sequence comprises the nucleic acid sequence of SEQ ID NO: 13, 14, or 15, or a nucleic acid sequence having at least about 85%, 90%, 95%, or 99% sequence identity thereof.

52. The method of any one of embodiments 46-51, wherein the intracellular signaling domain comprises a primary signaling domain, optionally wherein the primary signaling domain comprises a functional signaling domain derived from CD3 zeta, TCR zeta, FcR gamma, FcR beta, CD3 gamma, CD3 delta, CD3 epsilon, CD5, CD22, CD79a, CD79b, CD278 (ICOS), FcεRI, DAP10, DAP12, or CD66d, optionally wherein:
(a) the primary signaling domain comprises a functional signaling domain derived from CD3 zeta,
(b) the primary signaling domain comprises the amino acid sequence of SEQ ID NO: 9 or 10, or an amino acid sequence having at least about 85%, 90%, 95%, or 99% sequence identity thereof, or
(c) the nucleic acid molecule comprises a nucleic acid sequence encoding the primary signaling domain, wherein the nucleic acid sequence comprises the nucleic acid sequence of SEQ ID NO: 20 or 21, or a nucleic acid sequence having at least about 85%, 90%, 95%, or 99% sequence identity thereof.

53. The method of any one of embodiments 46-52, wherein the intracellular signaling domain comprises a costimulatory signaling domain, optionally wherein the costimulatory signaling domain comprises a functional signaling domain derived from a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, or a ligand that specifically binds with CD83, optionally wherein:
(a) the costimulatory signaling domain comprises a functional signaling domain derived from 4-1BB,
(b) the costimulatory signaling domain comprises the amino acid sequence of SEQ ID NO: 7, or an amino acid sequence having at least about 85%, 90%, 95%, or 99% sequence identity thereof, or
(c) the nucleic acid molecule comprises a nucleic acid sequence encoding the costimulatory signaling domain, wherein the nucleic acid sequence comprises the nucleic acid sequence of SEQ ID NO: 18, or a nucleic acid sequence having at least about 85%, 90%, 95%, or 99% sequence identity thereof.

54. The method of any one of embodiments 46-53, wherein the intracellular signaling domain comprises a functional signaling domain derived from 4-1BB and a functional signaling domain derived from CD3 zeta, optionally wherein the intracellular signaling domain comprises the amino acid sequence of SEQ ID NO: 7 (or an amino acid sequence having at least about 85%, 90%, 95%, or 99% sequence identity thereof) and the amino acid sequence of SEQ ID NO: 9 or 10 (or an amino acid sequence having at least about 85%, 90%, 95%, or 99% sequence identity thereof), optionally wherein the intracellular signaling domain comprises the amino acid sequence of SEQ ID NO: 7 and the amino acid sequence of SEQ ID NO: 9 or 10.

55. The method of any one of embodiments 46-54, wherein the CAR further comprises a leader sequence comprising the amino acid sequence of SEQ ID NO: 1.

56. A population of CAR-expressing cells (for example, autologous or allogeneic CAR-expressing T cells or NK cells) made by the method of any one of embodiments 1-55.

57. A pharmaceutical composition comprising the population of CAR-expressing cells of embodiment 56 and a pharmaceutically acceptable carrier.

58. A method of increasing an immune response in a subject, comprising administering the population of CAR-expressing cells of embodiment 56 or the pharmaceutical composition of embodiment 57 to the subject, thereby increasing an immune response in the subject.

59. A method of treating a cancer in a subject, comprising administering the population of CAR-expressing cells of embodiment 56 or the pharmaceutical composition of embodiment 57 to the subject, thereby treating the cancer in the subject.

60. The method of embodiment 59, wherein the cancer is a solid cancer, for example, chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof.

61. The method of embodiment 59, wherein the cancer is a liquid cancer, for example, chosen from: chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+large B-cell lymphoma, large B-cell lymphoma arising in HHV8-associated multicentric Castleman disease, primary effusion lymphoma, B-cell lymphoma, acute myeloid leukemia (AML), or unclassifiable lymphoma.

62. The method of any one of embodiments 58-61, further comprising administering a second therapeutic agent to the subject.

63. The method of any one of embodiments 58-62, wherein the population of CAR-expressing cells is administered at a dose determined based on the percentage of CAR-expressing cells measured in embodiment 39.

64. The population of CAR-expressing cells of embodiment 56 or the pharmaceutical composition of embodiment 57 for use in a method of increasing an immune response in a subject, said method comprising administering to the subject an effective amount of the population of CAR-expressing cells or an effective amount of the pharmaceutical composition.

65. The population of CAR-expressing cells of embodiment 56 or the pharmaceutical composition of embodiment 57 for use in a method of treating a cancer in a subject, said method comprising administering to the subject an effective amount of the population of CAR-expressing cells or an effective amount of the pharmaceutical composition.

66. A multispecific binding molecule comprising:
(i) an anti-CD3 binding domain,
(ii) an anti-CD28 binding domain comprising a heavy chain variable region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3 wherein:
   (a) the HCDR1, HCDR2, HCDR3, LCDR1, LCDR2, and LCDR3 comprise the amino acid sequences of SEQ ID NOs: 538, 539, 540, 530, 531, and 532, respectively;
   (b) the HCDR1, HCDR2, HCDR3, LCDR1, LCDR2, and LCDR3 comprise the amino acid sequences of SEQ ID NOs: 541, 539, 540, 530, 531, and 532, respectively;
   (c) the HCDR1, HCDR2, HCDR3, LCDR1, LCDR2, and LCDR3 comprise the amino acid sequences of SEQ ID NOs: 542, 543, 540, 533, 534, and 535, respectively; or
   (d) the HCDR1, HCDR2, HCDR3, LCDR1, LCDR2, and LCDR3 comprise the amino acid sequences of SEQ ID NOs: 544, 545, 546, 536, 534, and 532, respectively; and
(iii) an Fc region comprising:
   a L234A, L235A, S267K, and P329A mutation (LALASKPA), numbered according to the EU numbering system;
   a L234A, L235A, and P329G mutation (LALAPG), numbered according to the EU numbering system;
   a G237A, D265A, P329A, and S267K mutation (GADAPASK), numbered according to the EU numbering system;
   a L234A, L235A, and G237A mutation (LALGA), numbered according to the EU numbering system;
   a D265A, P329A, and S267K mutation (DAPASK), numbered according to the EU numbering system;
   a G237A, D265A, and P329A mutation (GADAPA), numbered according to the EU numbering system; or
   a L234A, L235A, and P329A mutation (LALAPA), numbered according to the EU numbering system.

67. The multispecific binding molecule of embodiment 66, wherein the anti-CD28 binding domain comprises:
(i) a VH comprising the amino acid sequence of SEQ ID NO: 547 or 548, or a sequence with at least 95% sequence identity to SEQ ID NO: 547 or 548;
(ii) a VL comprising the amino acid sequence of SEQ ID NO: 537, or a sequence with at least 95% sequence identity thereto;
(iii) a VH comprising the amino acid sequence of SEQ ID NO: 547 or a sequence with at least 95% sequence identity thereto, and a VL comprising the amino acid sequence of SEQ ID NO: 537, or a sequence with at least 95% sequence identity thereto; or
(iv) a VH comprising the amino acid sequence of SEQ ID NO: 548 or a sequence with at least 95% sequence identity thereto, and a VL comprising the amino acid sequence of SEQ ID NO: 537, or a sequence with at least 95% sequence identity thereto.

68. The multispecific binding molecule of embodiment 66 or 67, which further comprises a light chain constant region chosen from the light chain constant regions of kappa or lambda.

69. The multispecific binding molecule of any one of embodiments 66-68, wherein the Fc region comprises a CH2, a CH3, or both a CH2 and CH3, optionally wherein the CH2 and/or CH3 are selected from IgG1, IgG2, IgG3, or IgG4.

70. The multispecific binding molecule of any one of embodiments 66-69, wherein the anti-CD3 binding domain comprises:
(i) a HCDR1, HCDR2, HCDR3, LCDR1, LCDR2, and LCDR3 of an anti-CD3 antibody molecule of Table 27 (for example the anti-CD3 (1), anti-CD3 (2), anti-CD3 (3), or anti-CD3 (4)); or
(ii) the amino acid sequence of any VH and/or VL region of an anti-CD3 antibody molecule provided in Table 27 (for example the anti-CD3 (1), anti-CD3 (2), anti-CD3 (3), or anti-CD3 (4)), or an amino acid sequence at least 95% identical thereto.

71. A multispecific binding molecule, comprising a first binding domain and a second binding domain, wherein the multispecific binding molecule comprises:
(i) a first polypeptide comprising from N-terminal to C-terminal: VH of the first binding domain, VL of the first binding domain, VH of the second binding domain, CH1 and an Fc region, which comprises a CH2 and a CH3; and
(ii) a second polypeptide comprising from N-terminal to C-terminal: VL of the second binding domain and CL;
wherein the Fc region comprises:
   a L234A, L235A, S267K, and P329A mutation (LALASKPA), numbered according to the EU numbering system;
   a L234A, L235A, and P329G mutation (LALAPG), numbered according to the EU numbering system;
   a G237A, D265A, P329A, and S267K mutation (GADAPASK), numbered according to the EU numbering system;
   a L234A, L235A, and G237A mutation (LALGA), numbered according to the EU numbering system;
   a D265A, P329A, and S267K mutation (DAPASK), numbered according to the EU numbering system;
   a G237A, D265A, and P329A mutation (GADAPA), numbered according to the EU numbering system; or
a L234A, L235A, and P329A mutation (LALAPA), numbered according to the EU numbering system.

72. A multispecific binding molecule, comprising a first binding domain and a second binding domain, wherein the multispecific binding molecule comprises:
(i) a first polypeptide comprising from N-terminal to C-terminal: VH of the second binding domain, CH1, an Fc region comprising a CH2 and a CH3, VH of the first binding domain, and VL of the first binding domain; and
(ii) a second polypeptide comprising from N-terminal to C-terminal: VL of the second binding domain and CL;
wherein the Fc region comprises:
   a L234A, L235A, S267K, and P329A mutation (LALASKPA), numbered according to the EU numbering system;
   a L234A, L235A, and P329G mutation (LALAPG), numbered according to the EU numbering system;
   a G237A, D265A, P329A, and S267K mutation (GADAPASK), numbered according to the EU numbering system;
   a L234A, L235A, and G237A mutation (LALGA), numbered according to the EU numbering system;
   a D265A, P329A, and S267K mutation (DAPASK), numbered according to the EU numbering system;
   a G237A, D265A, and P329A mutation (GADAPA), numbered according to the EU numbering system; or
a L234A, L235A, and P329A mutation (LALAPA), numbered according to the EU numbering system.

73. A multispecific binding molecule, comprising a first binding domain and a second binding domain, wherein the multispecific binding molecule comprises:
(i) a first polypeptide comprising from N-terminal to C-terminal: VH of the second binding domain, CH1, VH of the first binding domain, VL of the first binding domain, and an Fc region comprising a CH2 and a CH3; and
(ii) a second polypeptide comprising from N-terminal to C-terminal: VL of the second binding domain and CL;
wherein the Fc region comprises:
   a L234A, L235A, S267K, and P329A mutation (LALASKPA), numbered according to the EU numbering system;
   a L234A, L235A, and P329G mutation (LALAPG), numbered according to the EU numbering system;
   a G237A, D265A, P329A, and S267K mutation (GADAPASK), numbered according to the EU numbering system;
   a L234A, L235A, and G237A mutation (LALGA), numbered according to the EU numbering system;
   a D265A, P329A, and S267K mutation (DAPASK), numbered according to the EU numbering system;
   a G237A, D265A, and P329A mutation (GADAPA), numbered according to the EU numbering system; or
a L234A, L235A, and P329A mutation (LALAPA), numbered according to the EU numbering system.

74. The multispecific binding molecule of any one of embodiments 71-73, wherein the first binding domain comprises an anti-CD3 binding domain and the second binding domain comprises a costimulatory molecule binding domain.

75. The multispecific binding molecule of any one of embodiments 71-73, wherein the first binding domain comprises a costimulatory molecule binding domain and the second binding domain comprises an anti-CD3 binding domain.

76. The multispecific binding molecule of embodiment 74 or 75, wherein the costimulatory molecule binding domain comprises an anti-CD2 binding domain or an anti-CD28 binding domain.

77. The method of any one of embodiments 1-55, or the multispecific binding molecule of any one of embodiments 66-76, wherein the multispecific binding molecule comprises:
(i) a heavy chain comprising the amino acid sequence of any of SEQ ID NOs: 794, 795, 798, 800, or 815-817 or an amino acid sequence having at least 95% sequence identity thereto; and/or
(ii) a light chain comprising the amino acid sequence of any of SEQ ID NOs: 673, 796, 797, 799, or 801, or an amino acid sequence having at least 95% sequence identity thereto.

78. The method of any one of embodiments 1-55, or the multispecific binding molecule of any one of embodiments 66-77, wherein the multispecific binding molecule comprises:
(i) a heavy chain comprising the amino acid sequence of SEQ ID NO: 794, or an amino acid sequence having at least 95% sequence identity thereto, and a light chain comprising the amino acid sequence of SEQ ID NO: 796, or an amino acid sequence having at least 95% sequence identity thereto;
(ii) a heavy chain comprising the amino acid sequence of SEQ ID NO: 794, or an amino acid sequence having at least 95% sequence identity thereto, and a light chain comprising the amino acid sequence of SEQ ID NO: 797, or an amino acid sequence having at least 95% sequence identity thereto;
(iii) a heavy chain comprising the amino acid sequence of SEQ ID NO: 795, or an amino acid sequence having at least 95% sequence identity thereto, and a light chain comprising the amino acid sequence of SEQ ID NO: 796, or an amino acid sequence having at least 95% sequence identity thereto;
(iv) a heavy chain comprising the amino acid sequence of SEQ ID NO: 795, or an amino acid sequence having at least 95% sequence identity thereto, and a light chain comprising the amino acid sequence of SEQ ID NO: 797, or an amino acid sequence having at least 95% sequence identity thereto;
(v) a heavy chain comprising the amino acid sequence of SEQ ID NO: 798, or an amino acid sequence having at least 95% sequence identity thereto, and a light chain comprising the amino acid sequence of SEQ ID NO: 799, or an amino acid sequence having at least 95% sequence identity thereto;
(vi) a heavy chain comprising the amino acid sequence of SEQ ID NO: 815, or an amino acid sequence having at least 95% sequence identity thereto, and a light chain comprising the amino acid sequence of SEQ ID NO: 799, or an amino acid sequence having at least 95% sequence identity thereto;
(vii) a heavy chain comprising the amino acid sequence of SEQ ID NO: 800, or an amino acid sequence having at least 95% sequence identity thereto, and a light chain comprising the amino acid sequence of SEQ ID NO: 801, or an amino acid sequence having at least 95% sequence identity thereto;
(viii) a heavy chain comprising the amino acid sequence of SEQ ID NO: 816, or an amino acid sequence having at least 95% sequence identity thereto, and a light chain comprising the amino acid sequence of SEQ ID NO: 673, or an amino acid sequence having at least 95% sequence identity thereto; or
(ix) a heavy chain comprising the amino acid sequence of SEQ ID NO: 817, or an amino acid sequence having at least 95% sequence identity thereto, and a light chain comprising the amino acid sequence of SEQ ID NO: 673, or an amino acid sequence having at least 95% sequence identity thereto.

79. A method of activating cells (e.g., immune effector cells, e.g., T cells), comprising contacting (for example, binding) a population of cells (for example, T cells, for example, T cells isolated from a frozen or fresh leukapheresis product) with the multispecific binding molecule of any one of embodiments 66-78.

80. A method of transducing cells (e.g., immune effector cells, e.g., T cells), comprising contacting (for example, binding) a population of cells (for example, T cells, for example, T cells isolated from a frozen or fresh leukapheresis product) with (i) the multispecific binding molecule of any one of embodiments 66-78, and (ii) a nucleic acid molecule, e.g., a nucleic acid molecule encoding a CAR.

Although methods and materials similar or equivalent to those described herein can be used in the practice or testing of the present invention, suitable methods and materials are described below. All publications, patent applications, patents, and other references (for example, sequence database reference numbers) mentioned herein are incorporated by reference in their entirety. For example, all GenBank, Unigene, and Entrez sequences referred to herein, for example, in any Table herein, are incorporated by reference. When one gene or protein references a plurality of sequence accession numbers, all of the sequence variants are encompassed.

In addition, the materials, methods, and examples are illustrative only and not intended to be limiting. Headings, sub-headings or numbered or lettered elements, for example, (a), (b), (i) etc., are presented merely for ease of reading. The use of headings or numbered or lettered elements in this document does not require the steps or elements be performed in alphabetical order or that the steps or elements are necessarily discrete from one another. Other features, objects, and advantages of the invention will be apparent from the description and drawings, and from the claims.

### BRIEF DESCRIPTION OF THE FIGURES

FIGS. 1A-1I: When purified T cells were incubated with cytokines, the naïve cells were the predominant population transduced. FIG. 1A is a graph showing exemplary cytokine process. FIG. 1B is a pair of graphs showing the percentages of CD3+ CAR+ cells at each indicated time point after transduction. FIG. 1C is a set of graphs showing the transduction within the CD3+CCR7+CD45RO− population in a CD3/CD28 bead stimulated populations (left) compared to cytokines only populations (right) in two independent donors. For the sample referred to as “Short stim IL7+IL15” in FIG. 1C, the cells were stimulated with beads for 2 days and then they were removed in the presence of IL7 and IL15. FIGS. 1D, 1E, and 1F are a set of flow cytometry graphs showing the transduction of T-cell subsets cultured with IL2 (FIG. 1D), IL15 (FIG. 1E), and IL7+IL15 (FIG. 1F) daily over a three-day period. FIG. 1G is a set of flow cytometry graphs showing the T cell differentiation on day 0 (left) and on day 1 (right) for CCR7 and CD45RO after stimulation with IL2 (upper right panel) or IL-15 (lower right panel). FIGS. 1H and 1I are a set of graphs showing the percentages of CD3+CCR7+RO−, CD3+CCR7+RO+, CD3+CCR7−RO+, and CD3+CCR7−RO− cells at day 0 or after 24-hour incubation with the indicated cytokines.

FIGS. 2A-2D: CARTs generated with one day of cytokine stimulation were functional. FIG. 2A: Purified T cells were transduced with a MOI of 1 and in all the cytokine conditions tested, the percentages of CAR-expressing cells observed at day 1 and day 10 were similar. The CARTs were generated within one day and expanded via CD3/CD28 beads after harvest for 9 days to mimic the in vivo setting. FIG. 2A is a pair of graphs showing the average percentages of CD3+ CAR+ cells under each condition for day 1 CARTs (left) and day 10 CARTs (right). FIG. 2B: The cytotoxicity capacity of the day 1 CARTs post expansion was measured using Nalm6 as the target cells. FIG. 2B is a graph showing % killing of CD19 positive Nalm6 cells by CARTs from each condition. Day 10 CARTs expanded using CD3/CD28 beads are marked as “Day 10.” All the other samples were day 1 CARTs. FIG. 2C: The secretion of IFNg of the expanded day 1 CARTs in response to Nalm6 target cells was tested. FIG. 2C is a graph showing the amount of IFN-gamma secretion by CARTs from each condition in the presence of CD19 positive or CD19 negative target cells. FIG. 2D: The proliferative capacity of the day 1 CARTs was tested by measurement of the incorporation of EDU. FIG. 2D is a graph showing the average percentages of EDU-positive cells for each condition. Similar to FIG. 2B, day 10 CARTs are marked as “Day 10” and all the other samples were day 1 CARTs.

FIGS. 3A-3B: The impact of MOI and media composition on transduction on day 0. FIG. 3A: Purified T cells were transduced with a range of MOIs from 1 to 10 in the presence of IL15, IL2+IL15, IL2+IL7, or IL7+IL15. Regardless of cytokine used, a linear increase in transduction was observed. FIG. 3A is a set of graphs where the percentages of CD3+ CAR+ cells are plotted against MOIs for each condition tested. FIG. 3B: The composition of the media impacted the transduction in the cytokine process. FIG. 3B is a pair of graphs showing the percentages of CD3+ CAR+ cells on day 1 (left) or day 8 (right) for each condition tested. “2.50” indicates a MOI of 2.50. “5.00” indicates a MOI of 5.00.

FIGS. 4A-4D: CAR T cells generated within 24 hours can eliminate tumor. FIG. 4A: Purified T cells were transduced with an anti-CD19 CAR and 24 hours later were harvested. FIG. 4A is a set of flow cytometry plots showing the transduction of T cells with the anti-CD19 CAR that were cultured with IL2, IL15 and IL7+IL15, illustrating the transduction with each cytokine condition. FIG. 4B: A graph showing average viability which was above 80% in all the conditions tested. FIG. 4C: The expansion of the day 1 CARTs in the peripheral blood is increased in vivo as compared to their day 10 counterparts. The percentage of live CD45+CD11b−CD3+CAR+ cells at indicated time points after infusion for each condition tested. The day 10 CARTs are marked as “D10 1e6” or “D10 5e6” and all the other samples were day 1 CARTs. FIG. 4D: The day 1 CARTs could eliminate tumor in vivo although with a delayed kinetics as compared to the day 10 CARTs. FIG. 4D is a graph showing total flux at indicated time points after tumor inoculation for each condition tested. CARTs were administered 4 days after tumor inoculation. The day 10 CARTs are marked as “5e6 d. 10” and all the other samples were day 1 CARTs.

FIGS. 5A-5B: The cytokine process was scalable. FIG. 5A: The T cells were enriched on a CliniMACS® Prodigy® and the B cell compartment was reduced to less than 1%. FIG. 5A is a set of flow cytometry plots showing the staining of cells with an anti-CD3 antibody (left) or an anti-CD19 antibody and an anti-CD14 antibody (right) for leukopak cells (upper) or cells post CD4+CD8+ enrichment (lower). FIG. 5B: Purified T cells from a frozen apheresis were transduced with an anti-CD19 CAR in either a 24 well plate or a PL30 bag post enrichment. The CARTs were harvested 24 hours later. FIG. 5B is a set of flow cytometry plots showing staining for CD3 and CAR of cells manufactured in the presence of either IL2 or hetIL-15 (IL15/sIL-15Ra).

FIGS. 6A-6C: The CARTs manufactured by the activation process showed superior anti-tumor efficacy in vivo. FIGS. 6A and 6B are graphs where tumor burden is plotted against the indicated time point after tumor implantation. “d.1” indicates CARTs manufactured using the activation process. “d.9” indicates CARTs manufactured with a traditional 9-day expansion protocol, serving as a positive control in this study. FIG. 6C is a set of representative images showing bioluminescence from mice.

FIGS. 7A-7B: IL6Rα and IL6Rβ expressing cells were enriched in less differentiated T cell population. Fresh T cells were stained for indicated surface antigens and examined for expression levels of IL6Rα and IL6Rβ on CD4 (FIG. 7A) and CD8 (FIG. 7B) T cell subsets.

FIGS. 8A and 8B: Both IL6Rα and IL6Rβ expressing cells were enriched in less differentiated T cell population. Fresh T cells were stained for indicated surface antigens and examined for expression levels of indicated surface antigens on CD4 (FIG. 8A) and CD8 (FIG. 8B) T cell subsets.

FIG. 9: IL6Rα expressing cells expressed surface markers of less differentiated T cells. Fresh T cells were stained for indicated surface antigens and examined for expression levels of various surface antigens in IL6Rα high, middle, and low expressing cell subsets.

FIG. 10: IL6Rβ expressing cells expressed surface markers of less differentiated T cells. Fresh T cells were stained for indicated surface antigens and examined for expression levels of various surface antigens in IL6Rβ high, middle, and low expressing cell subsets.

FIG. 11: IL6Rα but not IL6Rβ expression was down-regulated following TCR engagement. T cells were activated with αCD3αCD28 beads at day 0 and then examined for expression levels of IL6Rα and IL6Rβ at indicated time points.

FIG. 12: Fold expansion of cytokine treated T cells after TCR engagement. T cells were activated with αCD3αCD28 beads at day 0 in the presence of indicated cytokines and then monitored for cell numbers at indicated time points.

FIGS. 13A and 13B: IL2, IL7, and IL15 treatment did not affect cell size and viability after TCR engagement. T cells were activated with αCD3αCD28 beads at day 0 in the presence of indicated cytokines and then monitored for cell size (FIG. 13A) and viability (FIG. 13B) at indicated time points.

FIG. 14: Expression kinetics of various surface molecules on CD4 T cells after cytokine treatment. T cells were activated with αCD3αCD28 beads at day 0 in the presence of indicated cytokines and then examined for expression of various surface molecules by flow cytometry at indicated time points.

FIG. 15: Expression kinetics of various surface molecules on CD8 T cells after cytokine treatment. T cells were activated with αCD3αCD28 beads at day 0 in the presence of indicated cytokines and then examined for expression of various surface molecules by flow cytometry at indicated time points.

FIG. 16: IL6Rβ expression was mainly restricted on CD27 expressing T cell subsets after TCR engagement. T cells were activated with αCD3αCD28 beads at day 0 in the presence of indicated cytokines and then examined for IL6Rβ expression by flow cytometry at day 15.

FIG. 17: IL6Rβ expression was mainly restricted on CD57 non-expressing T cell subsets after TCR engagement. T cells were activated with αCD3αCD28 beads at day 0 in the presence of indicated cytokines and then examined for IL6Rβ expression by flow cytometry at day 25.

FIG. 18: Common γ-chain cytokine treated T cells produced functional cytokines at day 25. T cells were activated with αCD3αCD28 beads at day 0 in the presence of indicated cytokines and then examined for percentages of IL2, IFNγ, and TNFα producing T cells by flow cytometry at day 25.

FIGS. 19A and 19B: BCMA CAR expression on Day 1 using ARM at MOI=2.5 in T cells from two healthy donors. FIG. 19A is a panel of histograms showing BCMA CAR expression as measured by flow cytometry. FIG. 19B is a table listing reagents/conditions used in the flow cytometry analysis.

FIGS. 20A, 20B, and 20C: In vitro CAR expression kinetics from day 1 to day 4 of cells manufactured using the ARM process. CARs were stably expressed on day 3. FIG. 20A is a panel of histograms showing CAR expression at the indicated time points measured by flow cytometry. FIGS. 20B and 20C are graphs showing CAR+% and MFI values over time, respectively.

FIGS. 21A and 21B: In vivo triage in a KMS-11-luc multiple myeloma xenograft mouse model. Each mouse received 1.5E6 of day 1 CART product. FIG. 21A is a panel of histograms showing the day 1 and day 7 CAR expression in the CART cells. FIG. 21B is a graph showing the tumor kinetics (BLI level) after CART treatment.

FIGS. 22A, 22B, and 22C: In vivo triage of BCMA CAR using dose titration in a KMS-11-luc multiple myeloma xenograft mouse model. FIG. 22A is a panel of histograms showing the CAR expression at day 1 and day 3. FIG. 22B is a graph showing tumor intake kinetics after CART treatment using two different doses: a dose of 1.5e5 CAR+ T cells and a dose of 5e4 CAR+ T cells. The doses of CAR+ cells were normalized based on the day 3 CAR expression. FIG. 22C is a graph showing body weight kinetics over the course of this study.

FIGS. 23A, 23B, and 23C. FIGS. 23A and 23B are graphs showing percentage of T cell expressing the CAR on their cell surface (FIG. 23A) and mean fluorescence intensity (MFI) of CD3+CAR+ cells (FIG. 23B) observed over time (replicate efficiencies are averaged from the two flow panels shown in FIG. 23C). FIG. 23C is a panel of flow cytometry plots showing gating strategy for surface CAR expression on viable CD3+ cells, as based on UTD samples. Numbers in the plots indicate percent CAR positive.

FIGS. 24A and 24B. FIG. 24A is a graph showing end-to-end composition of the starting material (Prodigy® product) and at harvest at various time points after culture initiation. Naive (n), central memory (cm), effector memory (em), and effector (eff) subsets were defined by CD4, CD8, CCR7, and CD45RO surface expression or lack thereof. CD4 composition is indicated. For each time point, the left bar shows cell composition of the overall CD3+ population (bulk) and the right bar shows cell composition of the CAR+ fraction. FIG. 24B is a panel of flow cytometry plots showing gating strategy applied on live CD3+ events to determine overall transduction efficiency (top row), CD4/CD8 composition (middle row), and memory subsets (bottom row) within the overall CD3+ population (bulk) and the CAR+ fraction.

FIG. 25. Kinetics of T cell subsets expressing surface CAR over time, expressed as number of viable cells in the respective subsets.

FIG. 26. Viable cell recovery (number of viable cells recovered at harvest versus number of viable cells seeded) 12 to 24 hours after culture initiation as determined from pre-wash counts.

FIG. 27. Viability of rapid CARTs harvested 12 to 24 hours after culture initiation, as determined pre-wash and post-wash at the time of harvest.

FIGS. 28A, 28B, 28C, and 28D. FIG. 28A is a graph showing composition of the starting material (healthy donor leukopak; LKPK) and the T cell-enriched product as analyzed by flow cytometry. Numbers indicate % of parent (live, single cells). T: T cells; mono: monocytes; B: B cells; CD56 (NK): NK cells. FIG. 28B is a panel of flow cytometry plots showing gating strategy on live CD3+ events used to determine transduction rate (forward scatter FSC vs. CAR) and T cell subsets (CD4 vs. CD8 and CCR7 vs. CD45RO). For ARM-CD19 CAR (CD19 CART cells manufactured using the Activated Rapid Manufacturing (ARM) process) and TM-CD19 CAR (CD19 CART cells manufactured using the traditional manufacturing (TM) process), the left lower panels represent bulk cultures, while the right panels represent CAR+ T cells. “ARM-UTD” and “TM-UTD” refer to untransduced T cells (UTD) manufactured according to the ARM and the TM processes, respectively. Numbers in quadrants indicate % of parental population. Boxes in the TM-UTD and TM-CD19 CAR plots indicate skewing toward a T_{CM} phenotype for the TM process. Boxes in the ARM-UTD and ARM-CD19 CAR plots indicate the maintenance of naïve-like cells by the ARM process. NA: not applicable. FIG. 28C is a graph showing end-to-end T cell composition of ARM-CD19 CAR and TM-CD19 CAR. Composition is shown for “bulk” and “CAR+” populations where applicable. The percentage of the respective populations refers to % of parental, either CD3+ or CAR+CD3+ as applicable. The % of CD4 cells of the respective bulk or CAR+ population is indicated. LKPK: Leukopak starting material; 4 and 8: CD4+ and CD8+, respectively; eff: effector; em: effector memory; cm: central memory; n: naïve-like. Data is representative of 3 full-scale runs with 3 different healthy donors (n=3) and several small-scale runs used to optimize the process. FIG. 28D is a table showing the percentages shown in FIG. 28C.

FIGS. 29A, 29B, 29C, and 29D. Cytokine concentration in cell culture supernatants. IFN-γ (FIGS. 29A and 29B) and IL-2 (FIGS. 29C and 29D). FIGS. 29A and 29C: TM-CD19 CAR, ARM-CD19 CAR, and respective UTD were co-cultured with NALM6-WT (ALL), TMD-8 (DLBCL), or without cancer cells (T cells alone). Supernatant was collected 48 h later. FIGS. 29B and 29D: ARM-CD19 CAR was cocultured with NALM6-WT, NALM6-19KO (CD19-negative) or alone. Supernatant was collected after 24 h or 48 h. To further assess antigen-specific cytokine secretion, ARM-CD19 CAR was cultured alone for 24 h, washed and then co-cultured with target cells for 24 h. Data shown is derived from 2 healthy donor T cells and is representative of 2 experiments with three donors total.

FIGS. 30A, 30B, and 30C. FIG. 30A is a graph outlining the xenograft mouse model to study the anti-tumor activity of ARM-CD19 CAR. FIG. 30B is a panel of flow cytometry plots showing determination of CAR expression on ARM-CD19 CAR cells from a sentinel vial. ARM-CD19 CAR cells were cultured for the time period described in the figure, prior to flow-cytometry analysis. Gating for CAR expression was based on an isotype control (Iso) staining. FIG. 30C is a graph showing in vivo efficacy of ARM-CD19 CAR in the xenograft mouse model. NSG mice were injected with the pre-B ALL line NALM6, expressing the luciferase reporter gene; the tumor burden is expressed as total body luminescence (p/s), depicted as mean tumor burden with 95% confidence interval. On day 7 post tumor inoculation, mice were treated with ARM-CD19 CAR or TM-CD19 CAR at the respective doses (number of viable CAR+ T cells). High dose ARM-CD19 CAR group was terminated on day 33 due to onset of X-GVHD. Vehicle (PBS) and non-transduced T cells (UTD) served as negative controls. n=5 mice for all groups, except n=4 for ARM-UTD 1×10⁶ dose and all TM-CD19 CAR dose groups. Five xenograft studies were run with CAR-T cells generated from 5 different healthy donors, three of which included a comparison to TM-CD19 CAR.

FIGS. 31A, 31B, 31C, and 31D. Plasma cytokine levels of NALM6 tumor-bearing mice treated with ARM-CD19 CAR or TM-CD19 CAR at respective CAR-T cell doses. Mice were bled and plasma cytokine measured by MSD assay. IFN-γ (FIGS. 31A and 31B) and IL-2 (FIGS. 31C and 31D) are shown for mice treated with CAR-T (FIGS. 31A and 31C) or ARM- and TM-UTD cells (FIGS. 31B and 31D). Bars within each dose represent the mean cytokine level within the group at different time points (from left: day 4, 7, 10, 12, 16, 19, 23, 26). Horizontal bars and numbers indicate the fold-change comparisons between ARM-CD19 CAR (1×10⁶ dose group) and TM-CD19 CAR (0.5×10⁶ dose group) described in the text: 3-fold for IFN-γ; and 10-fold for IL-2. Groups taken down due to tumor burden or body weight loss do not show the last time points. Plasma cytokine levels were measured for 2 studies. no tum: no tumor.

FIG. 32. Time course of total and CAR+ T cell concentrations in NALM6 tumor-bearing mice treated with PBS vehicle, UTD, TM-CD19 CAR, or ARM-CD19 CAR. Blood samples were taken at 4, 7, 14, 21 and 28 days post CAR-T cell injection. Total T cells (CD3+, upper) and CAR+ T cell (CD3+CAR+, lower) concentrations were analyzed by flow cytometry at designed time points, depicted as mean cells with 95% confidence interval.

FIGS. 33A and 33B. IL-6 protein levels in three-party co-culture supernatants in μg/mL. ARM-CD19 CAR/K562 co-cultured cells (FIG. 33A) or TM-CD19 CAR/K562 cell co-cultured cells (FIG. 33B), for 6 or 24 hours incubated at different ratios (1:1 and 1:2.5), were then added to PMA-differentiated THP-1 cells for another 24 hours. Results from CAR-T cells co-cultured with K562-CD19 cells, CAR-T cells co-cultured with K562-Mesothelin cells, and CAR-T cells alone are shown. 1:5 ratios are not shown for clarity. ARM-CD19 CAR only and TM-CD19 CAR only designated bars represent CAR-T cell cultures (6 h, 24 h) without target cells. Mean+SEM, duplicates of n=1 (TM-CD19 CAR) and n=3 (ARM-CD19 CAR).

FIGS. 34A, 34B, and 34C. ARM process preserves BCMA CAR+ T cell stemness. PI61, R1G5 and BCMA10 CART cells manufactured using the ARM process were assessed for CAR expression at thaw (FIG. 34A) and 48 h post-thaw (FIG. 34B). CCR7/CD45RO markers were also assessed for the 48 h post-thaw product (FIG. 34C). Data shown is one representative from two experiments performed using two donor T cells.

FIGS. 35A and 35B. The TM process mainly resulted in central-memory T cells (TCM) (CD45RO+/CCR7+), while the naive-like T cell population is almost gone in the CAR+ T cells with TM process. PI61, R1G5 and BCMA10 CART cells manufactured using the TM process were assessed for CAR expression at day 9 (FIG. 35A). CCR7/CD45RO markers were also assessed at day 9 post-thaw product (FIG. 35B). Data shown is one representative from two experiments performed using two donor T cells.

FIGS. 36A, 36B, 36C, and 36D. ARM processed BCMA CAR-T cells demonstrates BCMA-specific activation and secretes higher levels of IL2 and IFN-γ. IL-2 and IFN-γ concentrations in cell culture supernatants. PI61, R1G5 and BCMA10 CART cells manufactured using the ARM or TM process, and respective UTD were co-cultured with KMS-11 at 2.5:1 ratio. Supernatants were collected 20 h later. For the ARM products, IFN-γ concentrations are shown in FIG. 36A and IL-2 concentrations are shown in FIG. 36B. For the TM products, IFN-γ concentrations are shown in FIG. 36C and IL-2 concentrations are shown in FIG. 36D. Data shown is one representative from two experiments performed using two donor T cells.

FIGS. 37A, 37B, and 37C. Single cell RNA-seq data for input cells (FIG. 37A), Day 1 cells (FIG. 37B), and Day 9 cells (FIG. 37C). The “nGene” graphs show the number of expressed genes per cell. The “nUMI” graphs show the number of unique molecular identifiers (UMIs) per cell.

FIGS. 38A, 38B, 38C, and 38D. T-Distributed Stochastic Neighbor Embedding (TSNE) plots comparing input cells (FIG. 38A), Day 1 cells (FIG. 38B), and Day 9 cells (FIG. 38C) for a proliferation signature, which was determined based on expression of genes CCNB1, CCND1, CCNE1, PLK1, and MK167. Each dot represents a cell in that sample. Cells shown as light grey do not express the proliferation genes whereas dark shaded cells express one or more of the proliferation genes. FIG. 38D is a violin plot showing the distribution of gene set scores for a gene set comprised of genes that characterize a resting vs. activated T cell state for Day 1 cells, Day 9 cells, and input cells. In FIG. 38D, a higher gene set score (Up resting vs. Down activated) indicates an increasing resting T cell phenotype, whereas a lower gene set score (Up resting vs. Down activated) indicates an increasing activated T cell phenotype. Input cells were overall in more of a resting state compared to Day 9 and Day 1 cells. Day 1 cells show the greatest activation gene set score.

FIGS. 39A, 39B, 39C, 39D and 39E. Gene set analysis for input cells, Day 1 cells, and Day 9 cells. In FIG. 39A, a higher gene set score for the gene set “Up TEM vs. Down TSCM” indicates an increasing effector memory T cell (TEM) phenotype of the cells in that sample, whereas a lower gene set score indicates an increasing stem cell memory T cell (TSCM) phenotype. In FIG. 39B, a higher gene set score for the gene set “Up Treg vs. Down Teff” indicates an increasing regulatory T cell (Treg) phenotype, whereas a lower gene set score indicates an increasing effector T cell (Teff) phenotype. In FIG. 39C, a lower gene set score for the gene set “Down stemness” indicates an increasing stemness phenotype. In FIG. 39D, a higher gene set score for the gene set “Up hypoxia” indicates an increasing hypoxia phenotype. In FIG. 39E, a higher gene set score for the gene set “Up autophagy” indicates an increasing autophagy phenotype. Day 1 cells looked similar to the input cells in terms of memory, stem-like and differentiation signature. Day 9 cells, on the other hand, show a higher enrichment for metabolic stress.

FIGS. 40A, 40B, and 40C. Gene cluster analysis for input cells. FIGS. 40A-40C are violin plots showing the gene set scores from gene set analysis of the four clusters of the input cells. Each dot overlaying the violin plots in FIGS. 40A-40C represents a cell's gene set score. In FIG. 40A, a higher gene set score of the gene set “Up Treg vs. Down Teff” indicates an increasing Treg cell phenotype, whereas a lower gene set score of the gene set “Up Treg vs. Down Teff” indicates an increasing Teff cell phenotype. In FIG. 40B, a higher gene set score of the gene set “Progressively up in memory differentiation” indicates an increasing late memory T cell phenotype, whereas a lower gene set score of the gene set “Progressively up in memory differentiation” indicates an increasing early memory T cell phenotype. In FIG. 40C, a higher gene set score of the gene set “Up TEM vs. Down TN” indicates an increasing effector memory T cell phenotype, whereas a lower gene set score of the gene set “Up TEM vs. Down TN” indicates an increasing naïve T cell phenotype. The cells in Cluster 3 are shown to be in a later memory, further differentiated T cell state compared to the cells in Cluster 1 and Cluster 2 which are in an early memory, less differentiated T cell state. Cluster 0 appears to be in an intermediate T cell state. Taken together, this data shows that there is a considerable level of heterogeneity within input cells.

FIGS. 41A, 41B, and 41C. TCR sequencing and measuring clonotype diversity. Day 9 cells have flatter distribution of clonotype frequencies (higher diversity).

FIG. 42 is a flow chart showing the design of a Phase I clinical trial testing BCMA CART cells manufactured using the ARM process in adult patients with relapsed and/or refractory multiple myeloma.

FIG. 43 is a graph showing FACS analyses for ARM-BCMA CAR expression at different collection time points post viral addition in the presence or absence of AZT at two different concentrations (30 μM and 100 μM). Lentiviral vector was added 1 h later prior to AZT treatment at the time of activation and cell seeding.

FIGS. 44A and 44B are graphs showing assessment of ARM-BCMA CAR for CAR expression at thaw (FIG. 44A) and 48 h post-thaw and CCR7/CD45RO markers at 48 h post-thaw product as well as day 9 for TM-BCMA CAR (FIG. 44B). Data shown is one representative from two experiments performed using T cells from two donors.

FIGS. 45A and 45B are graphs showing cytokine concentrations in cell culture supernatants. ARM-BCMA CAR and TM-BCMA CAR, and respective UTD were co-cultured with KMS-11. Supernatant was collected 24 h later. Data shown is one representative from two experiments performed using T cells from two donors.

FIG. 46 is a graph showing outline of xenograft efficacy study to test ARM-BCMA.

FIG. 47 is a graph comparing the efficacy of ARM-BCMA CAR with that of TM-BCMA CAR in a xenograft model. NSG mice were injected with MM cell line KMS11, expressing the luciferase reporter gene. The tumor burden is expressed as total body luminescence (p/s), depicted as mean tumor burden+SEM. On day 8 post tumor inoculation, mice were treated with ARM-BCMA CAR or TM-BCMA CAR at the respective doses (number of viable CAR+ T cells). Vehicle (PBS) and UTD T cells served as negative controls. N=5 mice for all groups, except N=4 for ARM-BCMA CAR (1e4 cells), PBS, and UTD groups.

FIGS. 48A, 48B, and 48C are graphs showing plasma IFN-γ kinetics of mice treated with ARM-BCMA CAR or TM-BCMA CAR. Plasma IFN-γ levels of KMS11-luc tumor-bearing mice treated with UTD, ARM-BCMA CAR, or TM-BCMA CAR at respective CAR-T doses. All IFN-γ levels were depicted as mean±SEM. Mice were bled and plasma cytokine measured by Meso Scale Discovery (MSD) assay.

FIG. 49 is a graph showing cellular kinetics of ARM-BCMA CAR and TM-BCMA CAR in vivo. Cellular kinetics in peripheral blood of KMS11 tumor-bearing mice treated with TM UTD, ARM UTD, ARM-BCMA CAR, and TM-BCMA CAR at different doses. Cell count is expressed as mean cell count+SD. On day 8 post tumor inoculation, mice were treated with ARM-BCMA CAR or TM-BCMA CAR at the respective doses (number of viable CAR+ T cells). Vehicle (PBS) and UTD T cells served as negative controls. Blood samples were taken at 7, 14, and 21 days post CAR-T injection and were analyzed by flow cytometry at designed time points. N=5 mice for all groups, except N=4 for ARM-BCMA CAR (1e4 cells), PBS, and UTD groups

FIG. 50A-C provide exemplary schema for bispecific antibodies, including single bispecific antibody schema (FIG. 50A), multimeric bispecific antibody schema (FIG. 50B), and a figure legend (FIG. 50C).

FIGS. 51A-B depict schema of the 17 different constructs comprising a CD3 antigen binding domain comprising a heavy and light chain derived from an anti-CD3 antibody and, in all but control Constructs 11, 14, and 17, an a CD28 or CD2 antigen binding domain, as noted, comprising a heavy and light chain derived from an anti-CD28 or CD2 antibody, respectively.

Construct 1 comprises an anti-CD3 scFv fused to an anti-CD2 Fab, which is further fused to an Fc region. Construct 1 comprises a first chain and a second chain. The first chain comprises, from the N-terminus to the C-terminus, anti-CD2 VL and CL. The second chain comprises, from the N-terminus to the C-terminus, anti-CD3 VH, (G4S) 4 linker (SEQ ID NO: 63), anti-CD3 VL, (G4S) 4 linker (SEQ ID NO: 63), anti-CD2 VH, CH1, CH2, and CH3. Construct 2 comprises an anti-CD3 scFv fused to an anti-CD28 Fab, which is further fused to an Fc region. Construct 2 comprises a first chain and a second chain. The first chain comprises, from the N-terminus to the C-terminus, anti-CD28 VL and CL. The second chain comprises, from the N-terminus to the C-terminus, anti-CD3 VH, (G4S) 4 linker (SEQ ID NO: 63), anti-CD3 VL, (G4S) 4 linker (SEQ ID NO: 63), anti-CD28 VH, CH1, CH2, and CH3.

Construct 3 comprises an anti-CD2 Fab fused to an Fc region, which is further fused to an anti-CD3 scFv. Construct 3 comprises a first chain and a second chain. The first chain comprises, from the N-terminus to the C-terminus, anti-CD2 VL and CL. The second chain comprises, from the N-terminus to the C-terminus, anti-CD2 VH, CH1, CH2, CH3, (G4S) 4 linker (SEQ ID NO: 63), anti-CD3 VH, (G4S) 4 linker (SEQ ID NO: 63), anti-CD3 VL. Construct 4 comprises an anti-CD28 Fab fused to an Fc region, which is further fused to an anti-CD3 scFv. Construct 4 comprises a first chain and a second chain. The first chain comprises, from the N-terminus to the C-terminus, anti-CD28 VL and CL. The second chain comprises, from the N-terminus to the C-terminus, anti-CD28 VH, CH1, CH2, CH3, (G4S) 4 linker (SEQ ID NO: 63), anti-CD3 VH, (G4S) 4 linker (SEQ ID NO: 63), anti-CD3 VL.

Construct 5 comprises an anti-CD2 Fab fused to an anti-CD3 scFv, which is further fused to an Fc region. Construct 5 comprises a first chain and a second chain. The first chain comprises, from the N-terminus to the C-terminus, anti-CD2 VL and CL. The second chain comprises, from the N-terminus to the C-terminus, anti-CD2 VH, CH1, (G4S) 2 linker (SEQ ID NO: 5), anti-CD3 VH, (G4S) 4 linker (SEQ ID NO: 63), anti-CD3 VL, (G4S) 4 linker (SEQ ID NO: 63), CH2, and CH3. Construct 6 comprises an anti-CD28 Fab fused to an anti-CD3 scFv, which is further fused to an Fc region. Construct 6 comprises a first chain and a second chain. The first chain comprises, from the N-terminus to the C-terminus, anti-CD28 VL and CL. The second chain comprises, from the N-terminus to the C-terminus, anti-CD28 VH, CH1, (G4S) 2 linker (SEQ ID NO: 5), anti-CD3 VH, (G4S) 4 linker (SEQ ID NO: 63), anti-CD3 VL, (G4S) 4 linker (SEQ ID NO: 63), CH2, and CH3.

Construct 7 comprises an anti-CD3 scFv fused to an Fc region, which is further fused to an anti-CD2 Fab. Construct 7 comprises a first chain and a second chain. The first chain comprises, from the N-terminus to the C-terminus, anti-CD2 VL and CL. The second chain comprises, from the N-terminus to the C-terminus, anti-CD3 VH, (G4S) 4 linker (SEQ ID NO: 63), anti-CD3 VL, (G4S) linker (SEQ ID NO: 25), CH2, CH3, (G4S) 4 linker (SEQ ID NO: 63), anti-CD2 VH, and CH1. Construct 8 comprises an anti-CD3 scFv fused to an Fc region, which is further fused to an anti-CD28 Fab. Construct 8 comprises a first chain and a second chain. The first chain comprises, from the N-terminus to the C-terminus, anti-CD28 VL and CL. The second chain comprises, from the N-terminus to the C-terminus, anti-CD3 VH, (G4S) 4 linker (SEQ ID NO: 63), anti-CD3 VL, (G4S) linker (SEQ ID NO: 25), CH2, CH3, (G4S) 4 linker (SEQ ID NO: 63), anti-CD28 VH, and CH1.

Construct 9 comprises an anti-CD2 Fab fused to a first Fc region and an anti-CD3 scFv fused to a second Fc region. Construct 9 comprises a first chain, a second chain, and a third chain. The first chain comprises, from the N-terminus to the C-terminus, anti-CD2 VL and CL. The second chain comprises, from the N-terminus to the C-terminus, anti-CD2 VH, CH1, CH2, and CH3. The third chain comprises, from the N-terminus to the C-terminus, anti-CD3 VH, (G4S) 4 linker (SEQ ID NO: 63), anti-CD3 VL, (G4S) linker (SEQ ID NO: 25), CH2, and CH3. Construct 10 comprises an anti-CD28 Fab fused to a first Fc region and an anti-CD3 scFv fused to a second Fc region. Construct 10 comprises a first chain, a second chain, and a third chain. The first chain comprises, from the N-terminus to the C-terminus, anti-CD28 VL and CL. The second chain comprises, from the N-terminus to the C-terminus, anti-CD28 VH, CH1, CH2, and CH3. The third chain comprises, from the N-terminus to the C-terminus, anti-CD3 VH, (G4S) 4 linker (SEQ ID NO: 63), anti-CD3 VL, (G4S) linker (SEQ ID NO: 25), CH2, and CH3.

Construct 11 comprises an anti-CD3 scFv fused to an Fc region. Construct 11 comprises a first chain and a second chain. The first chain comprises, from the N-terminus to the C-terminus, CH2 and CH3. The second chain comprises, from the N-terminus to the C-terminus, anti-CD3 VH, (G4S) 4 linker (SEQ ID NO: 63), anti-CD3 VL, (G4S) linker (SEQ ID NO: 25), CH2, and CH3.

Construct 12 comprises an anti-CD2 Fab fused to a first Fc region and an anti-CD3 scFv fused to a second Fc region. Construct 12 comprises a first chain, a second chain, and a third chain. The first chain comprises, from the N-terminus to the C-terminus, anti-CD2 VL and CL. The second chain comprises, from the N-terminus to the C-terminus, anti-CD2 VH, CH1, CH2, and CH3. The third chain comprises, from the N-terminus to the C-terminus, anti-CD3 VH, (G4S) 4 linker (SEQ ID NO: 63), anti-CD3 VL, (G4S) linker (SEQ ID NO: 25), CH2, CH3, (G4S) 3 linker (SEQ ID NO: 104), and Matrilin1. Construct 13 comprises an anti-CD28 Fab fused to a first Fc region and an anti-CD3 scFv fused to a second Fc region. Construct 13 comprises a first chain, a second chain, and a third chain. The first chain comprises, from the N-terminus to the C-terminus, anti-CD28 VL and CL. The second chain comprises, from the N-terminus to the C-terminus, anti-CD28 VH, CH1, CH2, and CH3. The third chain comprises, from the N-terminus to the C-terminus, anti-CD3 VH, (G4S) 4 linker (SEQ ID NO: 63), anti-CD3 VL, (G4S) linker (SEQ ID NO: 25), CH2, CH3, (G4S) 3 linker ((SEQ ID NO: 104), and Matrilin1.

Construct 14 comprises an anti-CD3 scFv fused to an Fc region. Construct 14 comprises a first chain and a second chain. The first chain comprises, from the N-terminus to the C-terminus, CH2 and CH3. The second chain comprises, from the N-terminus to the C-terminus, anti-CD3 VH, (G4S) 4 (SEQ ID NO: 63), linker, anti-CD3 VL, (G4S) linker (SEQ ID NO: 25), CH2, CH3, (G4S) 3 linker (SEQ ID NO: 104), and Matrilin1.

Construct 15 comprises an anti-CD2 Fab fused to a first Fc region and an anti-CD3 scFv fused to a second Fc region. Construct 15 comprises a first chain, a second chain, and a third chain. The first chain comprises, from the N-terminus to the C-terminus, anti-CD2 VL and CL. The second chain comprises, from the N-terminus to the C-terminus, anti-CD2 VH, CH1, CH2, and CH3. The third chain comprises, from the N-terminus to the C-terminus, anti-CD3 VH, (G4S) 4 linker (SEQ ID NO: 63), anti-CD3 VL, (G4S) linker (SEQ ID NO: 25), CH2, CH3, (G4S) linker (SEQ ID NO: 25), and the coiled-coil domain of cartilage oligomeric matrix protein (COMPcc). Construct 16 comprises an anti-CD28 Fab fused to a first Fc region and an anti-CD3 scFv fused to a second Fc region. Construct 16 comprises a first chain, a second chain, and a third chain. The first chain comprises, from the N-terminus to the C-terminus, anti-CD28 VL and CL. The second chain comprises, from the N-terminus to the C-terminus, anti-CD28 VH, CH1, CH2, and CH3. The third chain comprises, from the N-terminus to the C-terminus, anti-CD3 VH, (G4S) 4 linker (SEQ ID NO: 63), anti-CD3 VL, (G4S) linker (SEQ ID NO: 25), CH2, CH3, (G4S) linker (SEQ ID NO: 25), and COMPcc.

Construct 17 comprises an anti-CD3 scFv fused to an Fc region. Construct 17 comprises a first chain and a second chain. The first chain comprises, from the N-terminus to the C-terminus, CH2 and CH3. The second chain comprises, from the N-terminus to the C-terminus, anti-CD3 VH, (G4S) 4 linker (SEQ ID NO: 63), anti-CD3 VL, (G4S) linker (SEQ ID NO: 25), CH2, CH3, (G4S) linker (SEQ ID NO: 25), and COMPcc.

FIG. 52 provides images of T cells from brightfield microscopy on day 4, after use of 10 μg/mL of the constructs and positive control. The numbers at the upper left corner of each image refer to the name of the constructs tested. For example, “1” refers to Construct 1, “2” refers to Construct 2, and so on and so forth. “TA” stands for TransAct.

FIG. 53 shows the results of the IFN gamma and IL-2 readouts from the MSD for each of the 17 constructs and TransAct. The numbers on the x-axis refer to the name of the constructs tested. For example, “1” refers to Construct 1, “2” refers to Construct 2, and so on and so forth. “TA” stands for TransAct.

FIG. 54 shows percent transduction of an anti-CD19 CAR for each of the 17 constructs and TransAct. The numbers on the x-axis refer to the name of the constructs tested. For example, “1” refers to Construct 1, “2” refers to Construct 2, and so on and so forth. “TA” stands for TransAct.

FIG. 55 depicts CAR transduction as a function of valency or costimulatory molecule targeted (CD2/CD28). In FIGS. 55, F1, F2, F3, F4, F5, and F7 have a ligand valency of 2; F12 and F13 have a ligand valency of 3; and F15 and F16 have a ligand valency of 5. F1, F3, F5, F7, F12, and F15 bind to CD2, whereas F2, F4, F13, and F16 bind to CD28.

FIGS. 56A-56D show specific killing of tumor cells (calculated by subtracting average % kill in Nalm6 CD19KO cells from average % kill in Nalm6 WT cells) for CAR T-cells generated using Constructs 1 (F1), 3 (F3), 4 (F4), 5 (F5) versus TransAct (“TA”). “H” (in “3H” and “5H”) indicates an antibody concentration of 10 μg/mL; “M” (in “1M,” “3M,” “4M,” and “5M”) indicates an antibody concentration of 1 μg/mL; and “L” (in “1 L,” “3 L,” “4 L,” and “5 L”) indicates an antibody concentration of 0.1 μg/mL.

FIGS. 57A-B show secreted cytokine levels of CAR T-cells generated using Construct 1 (F1), 3 (F3), 4 (F4), or 5 (F5) or TransAct, when co-cultured with Nalm6 WT cells (“ARM vs. Nalm6 WT”) or Nalm6 CD19 knockout cells (“ARM vs. Nalm6 CD19 KO”).

FIG. 58 shows the tumor burden as a function of time in the Nalm6 xenograft mouse model treated with CAR transduced or untransduced cells from both donors.

FIG. 59 shows both CAR+ and CD3+ counts (per 20 μL) from mice treated with CAR transduced or untransduced cells from both donors. These counts were obtained from week 2 blood samples subjected to FACS analysis.

FIGS. 60A-60D show anti-tumor activity (FIGS. 60A-60B) and in vivo CAR expansion (FIGS. 60C-60D) by donor.

FIGS. 61A-61B shows the binding information (FIG. 61A) and configuration (FIG. 61B) of second generation stimulatory constructs. “F5 ANTI-CD3 (2)” refers to an F5 construct with an anti-CD3 binder based on ANTI-CD3 (2).

FIG. 62 shows the transduction efficiency of various stimulatory constructs, including those shown in FIG. 61A. “TA” stands for TransAct. TransAct was used at 0.1% by volume (1 μL for every 1000 μL of culture).

FIGS. 63A-63B show the binder information (FIG. 63A) and configuration (FIG. 63B) of third generation stimulatory constructs.

FIG. 64 shows the transduction efficiency of various stimulatory constructs, including those shown in FIG. 63A. “TA” stands for TransAct.

FIGS. 65A and 65B show specific killing of Nalm6 cells (FIG. 65A) and non-specific killing of FcγR-bearing PL21 cells (FIG. 65B). “F3 Fc-silent” refers to NEG2042 and “F4 Fc-silent” refers to NEG2043 in FIG. 63A.

FIGS. 66A and 66B. FIG. 66A shows the percentages of T cells expressing an anti-CD19 CAR (“CAR19+”; upper), an anti-BCMA CAR (“BCMA+”; middle), or co-expressing both CARs (“BCMA+CAR19+”; lower) following co-transduction with two vectors. FIG. 66B shows the percentages of T cells expressing an anti-CD22 CAR (“CAR22+”) or co-expressing anti-CD19 CAR and anti-CD22 CAR (“CAR19+22+”) following transduction with a dual CAR-encoding vector, as determined at two different time points post-manufacturing.

FIG. 67 shows the tumor reduction of CD19 CARs prepared using an Fc-silenced (LALASKPA) Anti-CD3 (4)/anti-CD28 (2) bispecific construct (SEQ ID NO: 794 and 796), Anti-CD3 (2)/anti-CD28 (2) construct (SEQ ID NO: 798 and 799), Anti-CD3 (4)/anti-CD28 (1) construct (SEQ ID NO: 800 and 801), or TransAct (“TA”) and compared to untransduced controls (“UTD”) and PBS.

FIGS. 68A and 68B show specific killing of Nalm6 cells (FIG. 68A) and non-specific killing of FcγR-bearing PL21 cells (FIG. 68B). TA stands for TransAct. In FIG. 68B, the tumor cells were co-cultured with either CART cells (“CART”) or T cells not expressing CAR (UTD).

FIG. 69 show non-specific killing of FcγR-bearing PL21 cells. TA stands for Trans Act.

FIGS. 70A-70C show BCMA CAR expression (FIG. 70A), T cell memory phenotype (FIG. 70B) and activation phenotype (FIG. 70C). BCMA CART cells were manufactured using Anti-CD3 (4)/anti-CD28 (2) bispecific (SEQ ID NO: 794 and 796) (5 μg/mL) or TransAct (“TA”). DO stands for Day 0, D1 stands for Day 1, EFF stands for effector T cells, EM stands for effector memory T cells, CM stands for central memory T cells, and N stands for naive T cells.

FIGS. 71A-71F show CD19 CAR expression (FIGS. 71A and 71D), T cell memory phenotype (FIGS. 71B and 71E) and activation phenotype (FIGS. 71C and 71F). FIGS. 71A-71C show data generated using T cells from a first donor and FIGS. 71D-71F show data generated using T cells from a second donor. D0 stands for Day 0, D1 stands for Day 1, EFF stands for effector T cells, EM stands for effector memory T cells, CM stands for central memory T cells, and N stands for naive T cells.

### DETAILED DESCRIPTION

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The compositions and methods of the present invention encompass polypeptides and nucleic acids having the sequences specified, or sequences substantially identical or similar thereto, for example, sequences at least 85%, 90%, or 95% identical or higher to the sequence specified. In the context of an amino acid sequence, the term “substantially identical” is used herein to refer to a first amino acid sequence that contains a sufficient or minimum number of amino acid residues that are i) identical to, or ii) conservative substitutions of aligned amino acid residues in a second amino acid sequence such that the first and second amino acid sequences can have a common structural domain and/or common functional activity, for example, amino acid sequences that contain a common structural domain having at least about 85%, 90%, 91%, 92%, 93%, 94%, 95%, 96%, 97%, 98% or 99% identity to a reference sequence, for example, a sequence provided herein.

In the context of a nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity, for example, nucleotide sequences having at least about 75%, 80%, 85%, 90%, 91%, 92%, 93%, 94%, 95%, 96%, 97%, 98% or 99% identity to a reference sequence, for example, a sequence provided herein.

The term “variant” refers to a polypeptide that has a substantially identical amino acid sequence to a reference amino acid sequence, or is encoded by a substantially identical nucleotide sequence. In some embodiments, the variant is a functional variant.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

The term cytokine (for example, IL-2, IL-7, IL-15, IL-21, or IL-6) includes full length, a fragment or a variant, for example, a functional variant, of a naturally-occurring cytokine (including fragments and functional variants thereof having at least 10%, 30%, 50%, or 80% of the activity, e.g., the immunomodulatory activity, of the naturally-occurring cytokine). In some embodiments, the cytokine has an amino acid sequence that is substantially identical (e.g., at least about 85%, 90%, 91%, 92%, 93%, 94%, 95%, 96%, 97%, 98% or 99% identity) to a naturally-occurring cytokine, or is encoded by a nucleotide sequence that is substantially identical (e.g., at least about 75%, 80%, 85%, 90%, 91%, 92%, 93%, 94%, 95%, 96%, 97%, 98% or 99% identity) to a naturally-occurring nucleotide sequence encoding a cytokine. In some embodiments, as understood in context, the cytokine further comprises a receptor domain, e.g., a cytokine receptor domain (e.g., an IL-15/IL-15R).

The term “Chimeric Antigen Receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, for example, comprise a chimeric fusion protein. In some embodiments, the domains in the CAR polypeptide construct are not contiguous with each other, for example, are in different polypeptide chains, for example, as provided in an RCAR as described herein.

In some embodiments, the cytoplasmic signaling domain comprises a primary signaling domain (for example, a primary signaling domain of CD3-zeta). In some embodiments, the cytoplasmic signaling domain further comprises one or more functional signaling domains derived from at least one costimulatory molecule as defined below. In some embodiments, the costimulatory molecule is chosen from 41BB (i.e., CD137), CD27, ICOS, and/or CD28. In some embodiments, the CAR comprises a chimeric fusion protein comprising an extracellular antigen recognition domain, a transmembrane domain and an intracellular signaling domain comprising a functional signaling domain derived from a stimulatory molecule. In some embodiments, the CAR comprises a chimeric fusion protein comprising an extracellular antigen recognition domain, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen recognition domain, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen recognition domain, a transmembrane domain and an intracellular signaling domain comprising at least two functional signaling domains derived from one or more costimulatory molecule(s) and a functional signaling domain derived from a stimulatory molecule. In some embodiments the CAR comprises an optional leader sequence at the amino-terminus (N-terminus) of the CAR fusion protein. In some embodiments, the CAR further comprises a leader sequence at the N-terminus of the extracellular antigen recognition domain, wherein the leader sequence is optionally cleaved from the antigen recognition domain (for example, an scFv) during cellular processing and localization of the CAR to the cellular membrane.

A CAR that comprises an antigen binding domain (for example, an scFv, a single domain antibody, or TCR (for example, a TCR alpha binding domain or TCR beta binding domain)) that targets a specific tumor marker X, wherein X can be a tumor marker as described herein, is also referred to as XCAR. For example, a CAR that comprises an antigen binding domain that targets BCMA is referred to as BCMA CAR. The CAR can be expressed in any cell, for example, an immune effector cell as described herein (for example, a T cell or an NK cell).

The term “signaling domain” refers to the functional portion of a protein which acts by transmitting information within the cell to regulate cellular activity via defined signaling pathways by generating second messengers or functioning as effectors by responding to such messengers.

The term “antibody,” as used herein, refers to a protein, or polypeptide sequence derived from an immunoglobulin molecule, which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an intact antibody, or recombinant variants thereof, and refers to the antigen binding domain, for example, an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, and Fv fragments, scFv antibody fragments, linear antibodies, single domain antibodies such as sdAb (either VL or VH), camelid VHH domains, and multi-specific molecules formed from antibody fragments such as a bivalent fragment comprising two or more, for example, two, Fab fragments linked by a disulfide bridge at the hinge region, or two or more, for example, two isolated CDR or other epitope binding fragments of an antibody linked. An antibody fragment can also be incorporated into single domain antibodies, maxibodies, minibodies, nanobodies, intrabodies, diabodies, triabodies, tetrabodies, v-NAR and bis-scFv (see, for example, Hollinger and Hudson, Nature Biotechnology 23:1126-1136, 2005). Antibody fragments can also be grafted into scaffolds based on polypeptides such as a fibronectin type III (Fn3) (see U.S. Pat. No. 6,703,199, which describes fibronectin polypeptide minibodies).

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, for example, with respect to the N-terminal and C-terminal ends of the polypeptide, the scFv may comprise VL-linker-VH or may comprise VH-linker-VL. In some embodiments, the scFv may comprise the structure of NH₂-V_{L}-linker-V_{H}—COOH or NH₂-V_{H}-linker-V_{L}-COOH.

The terms “complementarity determining region” or “CDR,” as used herein, refer to the sequences of amino acids within antibody variable regions which confer antigen specificity and binding affinity. For example, in general, there are three CDRs in each heavy chain variable region (for example, HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme), or a combination thereof. In a combined Kabat and Chothia numbering scheme, in some embodiments, the CDRs correspond to the amino acid residues that are part of a Kabat CDR, a Chothia CDR, or both.

The portion of the CAR composition of the invention comprising an antibody or antibody fragment thereof may exist in a variety of forms, for example, where the antigen binding domain is expressed as part of a polypeptide chain including, for example, a single domain antibody fragment (sdAb), a single chain antibody (scFv), or for example, a human or humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). In some embodiments, the antigen binding domain of a CAR composition of the invention comprises an antibody fragment. In some embodiments, the CAR comprises an antibody fragment that comprises an scFv.

As used herein, the term “binding domain” or “antibody molecule” (also referred to herein as “anti-target binding domain”) refers to a protein, for example, an immunoglobulin chain or fragment thereof, comprising at least one immunoglobulin variable domain sequence. The term “binding domain” or “antibody molecule” encompasses antibodies and antibody fragments. In some embodiments, an antibody molecule is a multispecific antibody molecule, for example, it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In some embodiments, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The terms “bispecific antibody” and “bispecific antibodies” refer to molecules that combine the antigen binding sites of two antibodies within a single molecule. Thus, a bispecific antibody is able to bind two different antigens simultaneously or sequentially. Methods for making bispecific antibodies are well known in the art. Various formats for combining two antibodies are also known in the art. Forms of bispecific antibodies of the invention include, but are not limited to, a diabody, a single-chain diabody, Fab dimerization (Fab-Fab), Fab-scFv, and a tandem antibody, as known to those of skill in the art.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The term “multispecific binding molecule” refers to a molecule that specifically binds to at least two antigens and comprise two or more antigen-binding domains. The antigen-binding domains can each independently be an antibody fragment (e.g., scFv, Fab, nanobody), a ligand, or a non-antibody derived binder (e.g., fibronectin, Fynomer, DARPin).

The term “monovalent” as used herein in the context of a multispecific binding molecule, antibody (e.g., bispecific antibody), or antibody fragment refers to a multispecific binding molecule, antibody (e.g., bispecific antibody), or antibody fragment in which there is a single antigen binding domain for each antigen to which the multispecific binding molecule, antibody (e.g., bispecific antibody), or antibody fragment binds.

The term “bivalent” as used herein in the context of a multispecific binding molecule, antibody (e.g., bispecific antibody), or antibody fragment refers to a multispecific binding molecule, antibody (e.g., bispecific antibody), or antibody fragment in which there are two antigen binding domains for each antigen to which the multispecific binding molecule, antibody (e.g., bispecific antibody), or antibody fragment binds.

The term “multimer” refers to an aggregate of a plurality of molecules (such as but not limited to antibodies (e.g. bispecific antibodies)), optionally conjugated to one another.

The term “conjugated to” refers to one or more molecules covalently or non-covalently bound together, optionally, directly or via linker.

The term “Fc silent” refers to an Fc domain that has been modified to have minimal interaction with effector cells, e.g., to reduce or eliminate the ability of a binding molecule to mediate antibody dependent cellular cytotoxicity (ADCC) and/or antibody dependent cellular phagocytosis (ADCP). Silenced effector functions may be obtained by mutation in the Fc region of the antibodies and have been described in the art, such as, but not limited to, LALA and N297A (Strohl, W., 2009, Curr. Opin. Biotechnol. vol. 20 (6): 685-691); and D265A (Baudino et al., 2008, J. Immunol. 181:6664-69) see also Heusser et al., WO2012065950. Unless otherwise specified herein, numbering of amino acid residues in the Fc region or constant region is according to the EU numbering system, also called the EU index, as described in Kabat, et al., Sequences of Proteins of Immunological Interest, 5th Ed. Public Health Service, National Institutes of Health, Bethesda, Md. (1991). Examples of Fc silencing mutations include the LALA mutant comprising L234A and L235A mutation in the IgG1 Fc amino acid sequence, DAPA (D265A, P329A) (see, e.g., U.S. Pat. No. 6,737,056), N297A, DANAPA (D265A, N297A, and P329A), and/or LALADANAPS (L234A, L235A, D265A, N297A and P331S). Further, non-limiting exemplary embodiments of silencing mutations include LALGA (L234A, L235A, and G237A), LALASKPA (L234A, L235A, S267K, and P329A), DAPASK (D265A, P329A, and S267K), GADAPA (G237A, D265A, and P329A), GADAPASK (G237A, D265A, P329A, and S267K), LALAPG (L234A, L235A, and P329G), and LALAPA (L234A, L235A, and P329A), wherein the amino acid residues are numbered according to the EU numbering system. It is understood that the terms “LALA,” “DAPA,” “DANAPA,” “LALADANAPS,” “LALAGA”, “LALASKPA”, “DAPASK”, “GADAPA”, “GADAPASK”, “LALAPG”, and “LALAPA” represent shorthand terminology for the different combinations of substitutions described in this paragraph rather than contiguous amino acid sequences.

The term “CD3/TCR complex” refers to a complex on the T-cell surface comprising a TCR including a TCR alpha and TCR beta chain; CD3 including one CD3 gamma chain, one CD3 delta chain, and two CD3 epsilon chains; and a zeta domain. UniProt accession numbers P01848 (TCR alpha, constant domain), P01850 (TCR beta, constant domain 1), A0A5B9 (TCR beta, constant domain 2), P09693 (CD3 gamma), P04234 (CD3 delta), P07766 (CD3 epsilon) provide exemplary human sequences for these chains, with the exception of the zeta chain, responsible for intracellular signaling, which is discussed in further detail below. Further relevant accession numbers include A0A075B662 (murine TCR alpha, constant domain), A0A0A6YWV4 and/or A0A075B5J3 (murine TCR beta, constant domain 1), A0A075B5J4 (murine TCR beta, constant domain 2), P11942 (murine CD3 gamma), P04235 (murine CD3 delta), P22646 (murine CD3 epsilon).

The term “CD28” refers to a T-cell specific glycoprotein CD28, also referred to as Tp44, as well as all alternate names thereof, which functions as a costimulatory molecule. UniProt accession number P10747 provides exemplary human CD28 amino acid sequences (see also HGNC: 1653, Entrez Gene: 940, Ensembl: ENSG00000178562, and OMIM: 186760). Further relevant CD28 sequences include UniProt accession number P21041 (murine CD28).

The term “ICOS” refers to inducible T-cell costimulator, also referred to as AILIM, CVID1, CD278, as well as all alternate names thereof, which functions as a costimulatory molecule. UniProt accession number Q9Y6W8 provides exemplary human ICOS amino acid sequences (see also HGNC: 5351, Entrez Gene: 29851, Ensembl: ENSG00000163600, and OMIM: 604558). Further relevant ICOS sequences include UniProt accession number Q9WVS0 (murine ICOS).

The term “CD27” refers to T-cell activation antigen CD27, Tumor necrosis factor receptor superfamily member 7, T14, T-cell activation antigen S152, Tp55, as well as alternate names thereof, which functions as a costimulatory molecule. UniProt accession number P26842 provides exemplary human CD27 amino acid sequences (see also HGNC: 11922, Entrez Gene: 939, Ensembl: ENSG00000139193, and OMIM: 186711). Further relevant CD27 sequences include UniProt accession number P41272 (murine CD27).

The term “CD25” refers to IL-2 subunit alpha, TAC antigen, p55, insulin dependent diabetes mellitus 10, IMD21, P55, TCGFR, as well as alternate names thereof, which functions as a growth factor receptor. UniProt accession number P01589 provides exemplary human CD25 amino acid sequences (see also HGNC: 6008, Entrez Gene: 3559, Ensembl: ENSG00000134460, and OMIM: 147730). Further relevant CD25 sequences include UniProt accession number P01590 (murine CD25).

The term “4-1BB” refers to CD137 or Tumor necrosis factor receptor superfamily member 9, as well as alternate names thereof, which functions as a costimulatory molecule. UniProt accession number Q07011 provides exemplary human 4-1BB amino acid sequences (see also HGNC: 11924, Entrez Gene: 3604, Ensembl: ENSG00000049249, and OMIM: 602250). Further relevant 4-1BB sequences include UniProt accession number P20334 (murine 4-1BB).

The term “IL6RA” refers to IL-6 receptor subunit alpha or CD126, as well as alternate names thereof, which functions as a growth factor receptor. UniProt accession number P08887 provides exemplary human IL6RA amino acid sequences (see also HGNC: 6019, Entrez Gene: 3570, Ensembl: ENSG00000160712, and OMIM: 147880 Further relevant IL6RA sequences include UniProt accession number P22272 (murine IL6RA).

The term “IL6RB” refers to IL-6 receptor subunit beta or CD130, as well as alternate names thereof, which functions as a growth factor receptor. UniProt accession number P40189 provides exemplary human IL6RB amino acid sequences. Further relevant IL6RB sequences include UniProt accession number Q00560 (murine IL6RB).

The term “CD2” refers to T-cell surface antigen T11/Leu-5/CD2, lymphocyte function antigen 2, T11, or erythrocyte/rosette/LFA-3 receptor, as well as alternate names thereof, which functions as a growth factor receptor. UniProt accession number P06729 provides exemplary human CD2 amino acid sequences (see also HGNC: 1639, Entrez Gene: 914, Ensembl: ENSG00000116824, and OMIM: 186990). Further relevant CD2 sequences include UniProt accession number P08920 (murine CD2).

The terms “anti-tumor effect” and “anti-cancer effect” are used interchangeably and refer to a biological effect which can be manifested by various means, including but not limited to, for example, a decrease in tumor volume or cancer volume, a decrease in the number of tumor cells or cancer cells, a decrease in the number of metastases, an increase in life expectancy, a decrease in tumor cell proliferation or cancer cell proliferation, a decrease in tumor cell survival or cancer cell survival, or amelioration of various physiological symptoms associated with the cancerous condition. An “anti-tumor effect” or “anti-cancer effect” can also be manifested by the ability of the peptides, polynucleotides, cells and antibodies of the invention in prevention of the occurrence of tumor or cancer in the first place.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some embodiments, allogeneic material from individuals of the same species may be sufficiently unlike genetically to interact antigenically.

The term “xenogeneic” refers to a graft derived from an animal of a different species.

The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, for example, by retransfusion. Thus, in the context of “an apheresis sample” refers to a sample obtained using apheresis.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some embodiments cancers treated by the methods described herein include multiple myeloma, Hodgkin's lymphoma or non-Hodgkin's lymphoma.

The terms “tumor” and “cancer” are used interchangeably herein, for example, both terms encompass solid and liquid, for example, diffuse or circulating, tumors. As used herein, the term “cancer” or “tumor” includes premalignant, as well as malignant cancers and tumors.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connotate or include a limitation to a particular process of producing the intracellular signaling domain, for example, it does not mean that, to provide the intracellular signaling domain, one must start with a CD3zeta sequence and delete unwanted sequence, or impose mutations, to arrive at the intracellular signaling domain.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative substitutions are ones in which the amino acid residue is replaced with an amino acid residue having a similar side chain. Families of amino acid residues having similar side chains have been defined in the art. These families include amino acids with basic side chains (for example, lysine, arginine, histidine), acidic side chains (for example, aspartic acid, glutamic acid), uncharged polar side chains (for example, glycine, asparagine, glutamine, serine, threonine, tyrosine, cysteine, tryptophan), nonpolar side chains (for example, alanine, valine, leucine, isoleucine, proline, phenylalanine, methionine), beta-branched side chains (for example, threonine, valine, isoleucine) and aromatic side chains (for example, tyrosine, phenylalanine, tryptophan, histidine). Thus, one or more amino acid residues within a CAR of the invention can be replaced with other amino acid residues from the same side chain family and the altered CAR can be tested using the functional assays described herein.

The term “stimulation” in the context of stimulation by a stimulatory and/or costimulatory molecule refers to a response, for example, a primary or secondary response, induced by binding of a stimulatory molecule (for example, a TCR/CD3 complex) and/or a costimulatory molecule (for example, CD28 or 4-1BB) with its cognate ligand thereby mediating a signal transduction event, such as, but not limited to, signal transduction via the TCR/CD3 complex. Stimulation can mediate altered expression of certain molecules and/or reorganization of cytoskeletal structures, and the like.

The term “stimulatory molecule,” refers to a molecule expressed by a T cell that provides the primary cytoplasmic signaling sequence(s) that regulate primary activation of the TCR complex in a stimulatory way for at least some aspect of the T cell signaling pathway. In some embodiments, the ITAM-containing domain within the CAR recapitulates the signaling of the primary TCR independently of endogenous TCR complexes. In some embodiments, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing primary cytoplasmic signaling sequence that is of particular use in the invention includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”), FcεRI and CD66d, DAP10 and DAP12. In a specific CAR of the invention, the intracellular signaling domain in any one or more CARS of the invention comprises an intracellular signaling sequence, for example, a primary signaling sequence of CD3-zeta. The term “antigen presenting cell” or “APC” refers to an immune system cell such as an accessory cell (for example, a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

An “intracellular signaling domain,” as the term is used herein, refers to an intracellular portion of a molecule. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The intracellular signaling domain generates a signal that promotes an immune effector function of the CAR containing cell, for example, a CART cell. Examples of immune effector function, for example, in a CART cell, include cytolytic activity and helper activity, including the secretion of cytokines.

In some embodiments, the intracellular signaling domain can comprise a primary intracellular signaling domain. Exemplary primary intracellular signaling domains include those derived from the molecules responsible for primary stimulation, or antigen dependent simulation. In some embodiments, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, CD278 (also known as “ICOS”), FcεRI, CD66d, DAP10 and DAP12.

The term “zeta” or alternatively “zeta chain”, “CD3-zeta” or “TCR-zeta” refers to CD247. Swiss-Prot accession number P20963 provides exemplary human CD3 zeta amino acid sequences. A “zeta stimulatory domain” or alternatively a “CD3-zeta stimulatory domain” or a “TCR-zeta stimulatory domain” refers to a stimulatory domain of CD3-zeta or a variant thereof (for example, a molecule having mutations, for example, point mutations, fragments, insertions, or deletions). In some embodiments, the cytoplasmic domain of zeta comprises residues 52 through 164 of GenBank Acc. No. BAG36664.1 or a variant thereof (for example, a molecule having mutations, for example, point mutations, fragments, insertions, or deletions). In some embodiments, the “zeta stimulatory domain” or a “CD3-zeta stimulatory domain” is the sequence provided as SEQ ID NO: 9 or 10, or a variant thereof (for example, a molecule having mutations, for example, point mutations, fragments, insertions, or deletions).

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class I molecule, TNF receptor proteins, Immunoglobulin-like proteins, cytokine receptors, integrins, signaling lymphocytic activation molecules (SLAM proteins), activating NK cell receptors, BTLA, Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, and a ligand that specifically binds with CD83.

A costimulatory intracellular signaling domain refers to the intracellular portion of a costimulatory molecule.

The intracellular signaling domain can comprise the entire intracellular portion, or the entire native intracellular signaling domain, of the molecule from which it is derived, or a functional fragment thereof.

A “4-1BB costimulatory domain” refers to a costimulatory domain of 4-1BB, or a variant thereof (for example, a molecule having mutations, for example, point mutations, fragments, insertions, or deletions). In some embodiments, the “4-1BB costimulatory domain” is the sequence provided as SEQ ID NO: 7 or a variant thereof (for example, a molecule having mutations, for example, point mutations, fragments, insertions, or deletions).

“Immune effector cell,” as that term is used herein, refers to a cell that is involved in an immune response, for example, in the promotion of an immune effector response. Examples of immune effector cells include T cells, for example, alpha/beta T cells and gamma/delta T cells, B cells, natural killer (NK) cells, natural killer T (NKT) cells, mast cells, and myeloic-derived phagocytes.

“Immune effector function or immune effector response,” as that term is used herein, refers to function or response, for example, of an immune effector cell, that enhances or promotes an immune attack of a target cell. For example, an immune effector function or response refers a property of a T or NK cell that promotes killing or the inhibition of growth or proliferation, of a target cell. In the case of a T cell, primary stimulation and costimulation are examples of immune effector function or response.

The term “effector function” refers to a specialized function of a cell. Effector function of a T cell, for example, may be cytolytic activity or helper activity including the secretion of cytokines.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, IRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence. In some embodiments, expression comprises translation of an mRNA introduced into a cell.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (for example, naked or contained in liposomes) and viruses (for example, lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17 (8): 1453-1464 (2009). Other examples of lentivirus vectors that may be used in the clinic, include but are not limited to, for example, the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, for example, between two nucleic acid molecules, such as, two DNA molecules or two RNA molecules, or between two polypeptide molecules. When a subunit position in both of the two molecules is occupied by the same monomeric subunit; for example, if a position in each of two DNA molecules is occupied by adenine, then they are homologous or identical at that position. The homology between two sequences is a direct function of the number of matching or homologous positions; for example, if half (for example, five positions in a polymer ten subunits in length) of the positions in two sequences are homologous, the two sequences are 50% homologous; if 90% of the positions (for example, 9 of 10), are matched or homologous, the two sequences are 90% homologous.

“Humanized” forms of non-human (for example, murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321:522-525, 1986; Reichmann et al., Nature, 332:323-329, 1988; Presta, Curr. Op. Struct. Biol., 2:593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, for example, where necessary to join two protein coding regions, are in the same reading frame.

The term “parenteral” administration of an immunogenic composition includes, for example, subcutaneous (s.c.), intravenous (i.v.), intramuscular (i.m.), or intrasternal injection, intratumoral, or infusion techniques.

The term “nucleic acid,” “nucleic acid molecule,” “polynucleotide,” or “polynucleotide molecule” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. In some embodiments, a “nucleic acid,” “nucleic acid molecule,” “polynucleotide,” or “polynucleotide molecule” comprise a nucleotide/nucleoside derivative or analog. Unless otherwise indicated, a particular nucleic acid sequence also implicitly encompasses conservatively modified variants thereof (for example, degenerate codon substitutions, for example, conservative substitutions), alleles, orthologs, SNPs, and complementary sequences as well as the sequence explicitly indicated. Specifically, degenerate codon substitutions, for example, conservative substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “cancer associated antigen,” “tumor antigen,” “hyperproliferative disorder antigen,” and “antigen associated with a hyperproliferative disorder” interchangeably refer to antigens that are common to specific hyperproliferative disorders. In some embodiments, these terms refer to a molecule (typically a protein, carbohydrate or lipid) that is expressed on the surface of a cancer cell, either entirely or as a fragment (for example, MHC/peptide), and which is useful for the preferential targeting of a pharmacological agent to the cancer cell. In some embodiments, a tumor antigen is a marker expressed by both normal cells and cancer cells, for example, a lineage marker, for example, CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (for example, MHC/peptide), and not synthesized or expressed on the surface of a normal cell. In some embodiments, the hyperproliferative disorder antigens of the present invention are derived from, cancers including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer (for example, castrate-resistant or therapy-resistant prostate cancer, or metastatic prostate cancer), ovarian cancer, pancreatic cancer, and the like, or a plasma cell proliferative disorder, for example, asymptomatic myeloma (smoldering multiple myeloma or indolent myeloma), monoclonal gammopathy of undetermined significance (MGUS), Waldenstrom's macroglobulinemia, plasmacytomas (for example, plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome). In some embodiments, the CARs of the present invention include CARs comprising an antigen binding domain (for example, antibody or antibody fragment) that binds to a MHC presented peptide. Normally, peptides derived from endogenous proteins fill the pockets of Major histocompatibility complex (MHC) class I molecules and are recognized by T cell receptors (TCRs) on CD8+T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, for example, Sastry et al., J Virol. 2011 85 (5): 1935-1942; Sergeeva et al., Blood, 2011 117 (16): 4262-4272; Verma et al., J Immunol 2010 184 (4): 2156-2165; Willemsen et al., Gene Ther 2001 8 (21): 1601-1608; Dao et al., Sci Transl Med 2013 5 (176): 176ra33; Tassev et al., Cancer Gene Ther 2012 19 (2): 84-100). For example, TCR-like antibody can be identified from screening a library, such as a human scFv phage displayed library.

The term “tumor-supporting antigen” or “cancer-supporting antigen” interchangeably refer to a molecule (typically a protein, carbohydrate or lipid) that is expressed on the surface of a cell that is, itself, not cancerous, but supports the cancer cells, for example, by promoting their growth or survival for example, resistance to immune cells. Exemplary cells of this type include stromal cells and myeloid-derived suppressor cells (MDSCs). The tumor-supporting antigen itself need not play a role in supporting the tumor cells so long as the antigen is present on a cell that supports cancer cells.

The term “flexible polypeptide linker” or “linker” as used in the context of an scFv refers to a peptide linker that consists of amino acids such as glycine and/or serine residues used alone or in combination, to link variable heavy and variable light chain regions together. In some embodiments, the flexible polypeptide linker is a Gly/Ser linker and comprises the amino acid sequence (Gly-Gly-Gly-Ser) n, where n is a positive integer equal to or greater than 1 (SEQ ID NO: 41). For example, n=1, n=2, n=3. n=4, n=5 and n=6, n=7, n=8, n=9 and n=10 In some embodiments, the flexible polypeptide linkers include, but are not limited to, (Gly4 Ser) 4 (SEQ ID NO: 27) or (Gly4 Ser) 3 (SEQ ID NO: 28). In some embodiments, the linkers include multiple repeats of (Gly2Ser), (GlySer) or (Gly3Ser) (SEQ ID NO: 29). Also included within the scope of the invention are linkers described in WO2012/138475, incorporated herein by reference.

As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA that has been synthesized in vitro. In some embodiments the RNA is mRNA. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In some embodiments of a construct for transient expression, the poly(A) is between 50 and 5000 (SEQ ID NO: 30). In some embodiments the poly(A) is greater than 64. In some embodiments the poly(A) is greater than 100. In some embodiments the poly(A) is greater than 300. In some embodiments the poly(A) is greater than 400. poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (for example, one or more therapeutic agents such as a CAR of the invention). In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”—refer to the inhibition of the progression of a proliferative disorder, either physically by, for example, stabilization of a discernible symptom, physiologically by, for example, stabilization of a physical parameter, or both. In other embodiments the terms “treat”, “treatment” and “treating” refer to the reduction or stabilization of tumor size or cancerous cell count.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (for example, mammals, for example, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In some embodiments, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a cognate binding partner (for example, a stimulatory and/or costimulatory molecule present on a T cell) protein present in a sample, but which antibody or ligand does not substantially recognize or bind other molecules in the sample.

“Regulatable chimeric antigen receptor (RCAR),” as used herein, refers to a set of polypeptides, typically two in the simplest embodiments, which when in an immune effector cell, provides the cell with specificity for a target cell, typically a cancer cell, and with intracellular signal generation. In some embodiments, an RCAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined herein in the context of a CAR molecule. In some embodiments, the set of polypeptides in the RCAR are not contiguous with each other, for example, are in different polypeptide chains. In some embodiments, the RCAR includes a dimerization switch that, upon the presence of a dimerization molecule, can couple the polypeptides to one another, for example, can couple an antigen binding domain to an intracellular signaling domain. In some embodiments, the RCAR is expressed in a cell (for example, an immune effector cell) as described herein, for example, an RCAR-expressing cell (also referred to herein as “RCARX cell”). In some embodiments the RCARX cell is a T cell and is referred to as a RCART cell. In some embodiments the RCARX cell is an NK cell, and is referred to as a RCARN cell. The RCAR can provide the RCAR-expressing cell with specificity for a target cell, typically a cancer cell, and with regulatable intracellular signal generation or proliferation, which can optimize an immune effector property of the RCAR-expressing cell. In embodiments, an RCAR cell relies at least in part, on an antigen binding domain to provide specificity to a target cell that comprises the antigen bound by the antigen binding domain.

“Membrane anchor” or “membrane tethering domain”, as that term is used herein, refers to a polypeptide or moiety, for example, a myristoyl group, sufficient to anchor an extracellular or intracellular domain to the plasma membrane.

“Switch domain,” as that term is used herein, for example, when referring to an RCAR, refers to an entity, typically a polypeptide-based entity, that, in the presence of a dimerization molecule, associates with another switch domain. The association results in a functional coupling of a first entity linked to, for example, fused to, a first switch domain, and a second entity linked to, for example, fused to, a second switch domain. A first and second switch domain are collectively referred to as a dimerization switch. In embodiments, the first and second switch domains are the same as one another, for example, they are polypeptides having the same primary amino acid sequence and are referred to collectively as a homodimerization switch. In embodiments, the first and second switch domains are different from one another, for example, they are polypeptides having different primary amino acid sequences, and are referred to collectively as a heterodimerization switch. In embodiments, the switch is intracellular. In embodiments, the switch is extracellular. In embodiments, the switch domain is a polypeptide-based entity, for example, FKBP or FRB-based, and the dimerization molecule is small molecule, for example, a rapalogue. In embodiments, the switch domain is a polypeptide-based entity, for example, an scFv that binds a myc peptide, and the dimerization molecule is a polypeptide, a fragment thereof, or a multimer of a polypeptide, for example, a myc ligand or multimers of a myc ligand that bind to one or more myc scFvs. In embodiments, the switch domain is a polypeptide-based entity, for example, myc receptor, and the dimerization molecule is an antibody or fragments thereof, for example, myc antibody.

“Dimerization molecule,” as that term is used herein, for example, when referring to an RCAR, refers to a molecule that promotes the association of a first switch domain with a second switch domain. In embodiments, the dimerization molecule does not naturally occur in the subject or does not occur in concentrations that would result in significant dimerization. In embodiments, the dimerization molecule is a small molecule, for example, rapamycin or a rapalogue, for example, RAD001.

The term “low, immune enhancing, dose” when used in conjunction with an mTOR inhibitor, for example, an allosteric mTOR inhibitor, for example, RAD001 or rapamycin, or a catalytic mTOR inhibitor, refers to a dose of mTOR inhibitor that partially, but not fully, inhibits mTOR activity, for example, as measured by the inhibition of P70 S6 kinase activity. Methods for evaluating mTOR activity, for example, by inhibition of P70 S6 kinase, are discussed herein. The dose is insufficient to result in complete immune suppression but is sufficient to enhance the immune response. In some embodiments, the low, immune enhancing, dose of mTOR inhibitor results in a decrease in the number of PD-1 positive T cells and/or an increase in the number of PD-1 negative T cells, or an increase in the ratio of PD-1 negative T cells/PD-1 positive T cells. In some embodiments, the low, immune enhancing, dose of mTOR inhibitor results in an increase in the number of naive T cells. In some embodiments, the low, immune enhancing, dose of mTOR inhibitor results in one or more of the following:
an increase in the expression of one or more of the following markers: CD62L^{high}, CD127^{high}, CD27⁺, and BCL2, for example, on memory T cells, for example, memory T cell precursors;
a decrease in the expression of KLRG1, for example, on memory T cells, for example, memory T cell precursors; and
an increase in the number of memory T cell precursors, for example, cells with any one or combination of the following characteristics: increased CD62L^{high}, increased CD127^{high}, increased CD27⁺, decreased KLRG1, and increased BCL2;
wherein any of the changes described above occurs, for example, at least transiently, for example, as compared to a non-treated subject.

“Refractory” as used herein refers to a disease, for example, cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

“Relapsed” or “relapse” as used herein refers to the return or reappearance of a disease (for example, cancer) or the signs and symptoms of a disease such as cancer after a period of improvement or responsiveness, for example, after prior treatment of a therapy, for example, cancer therapy. The initial period of responsiveness may involve the level of cancer cells falling below a certain threshold, for example, below 20%, 1%, 10%, 5%, 4%, 3%, 2%, or 1%. The reappearance may involve the level of cancer cells rising above a certain threshold, for example, above 20%, 1%, 10%, 5%, 4%, 3%, 2%, or 1%. For example, for example, in the context of B-ALL, the reappearance may involve, for example, a reappearance of blasts in the blood, bone marrow (>5%), or any extramedullary site, after a complete response. A complete response, in this context, may involve <5% BM blast. More generally, in some embodiments, a response (for example, complete response or partial response) can involve the absence of detectable MRD (minimal residual disease). In some embodiments, the initial period of responsiveness lasts at least 1, 2, 3, 4, 5, or 6 days; at least 1, 2, 3, or 4 weeks; at least 1, 2, 3, 4, 6, 8, 10, or 12 months; or at least 1, 2, 3, 4, or 5 years.

Ranges: throughout this disclosure, various embodiments of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98%, or 99% identity, and includes subranges such as 96-99%, 96-98%, 96-97%, 97-99%, 97-98%, and 98-99% identity. This applies regardless of the breadth of the range.

A “gene editing system” as the term is used herein, refers to a system, for example, one or more molecules, that direct and effect an alteration, for example, a deletion, of one or more nucleic acids at or near a site of genomic DNA targeted by said system. Gene editing systems are known in the art and are described more fully below.

Administered “in combination”, as used herein, means that two (or more) different treatments are delivered to the subject during the course of the subject's affliction with the disorder, for example, the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, for example, an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The term “depletion” or “depleting”, as used interchangeably herein, refers to the decrease or reduction of the level or amount of a cell, a protein, or macromolecule in a sample after a process, for example, a selection step, for example, a negative selection, is performed. The depletion can be a complete or partial depletion of the cell, protein, or macromolecule. In some embodiments, the depletion is at least a 1%, 2%, 5%, 10%, 15%, 20%, 25%, 30%, 35%, 40%, 45%, 50%, 55%, 60%, 65%, 70%, 75%, 80%, 85%, 90%, 95%, or 99% decrease or reduction of the level or amount of a cell, a protein, or macromolecule, as compared to the level or amount of the cell, protein or macromolecule in the sample before the process was performed.

As used herein, a “naïve T cell” refers to a T cell that is antigen-inexperienced. In some embodiments, an antigen-inexperienced T cell has encountered its cognate antigen in the thymus but not in the periphery. In some embodiments, naïve T cells are precursors of memory cells. In some embodiments, naïve T cells express both CD45RA and CCR7, but do not express CD45RO. In some embodiments, naïve T cells may be characterized by expression of CD62L, CD27, CCR7, CD45RA, CD28, and CD127, and the absence of CD95 or CD45RO isoform. In some embodiments, naïve T cells express CD62L, IL-7 receptor-α, IL-6 receptor, and CD132, but do not express CD25, CD44, CD69, or CD45RO. In some embodiments, naïve T cells express CD45RA, CCR7, and CD62L and do not express CD95 or IL-2 receptor β. In some embodiments, surface expression levels of markers are assessed using flow cytometry.

The term “central memory T cells” refers to a subset of T cells that in humans are CD45RO positive and express CCR7. In some embodiments, central memory T cells express CD95. In some embodiments, central memory T cells express IL-2R, IL-7R and/or IL-15R. In some embodiments, central memory T cells express CD45RO, CD95, IL-2 receptor β, CCR7, and CD62L. In some embodiments, surface expression levels of markers are assessed using flow cytometry.

The term “stem memory T cells,” “stem cell memory T cells,” “stem cell-like memory T cells,” “memory stem T cells,” “T memory stem cells,” “T stem cell memory cells” or “TSCM cells” refers to a subset of memory T cells with stem cell-like ability, for example, the ability to self-renew and/or the multipotent capacity to reconstitute memory and/or effector T cell subsets. In some embodiments, stem memory T cells express CD45RA, CD95, IL-2 receptor β, CCR7, and CD62L. In some embodiments, surface expression levels of markers are assessed using flow cytometry. In some embodiments, exemplary stem memory T cells are disclosed in Gattinoni et al., Nat Med. 2017 Jan. 6; 23 (1): 18-27, herein incorporated by reference in its entirety.

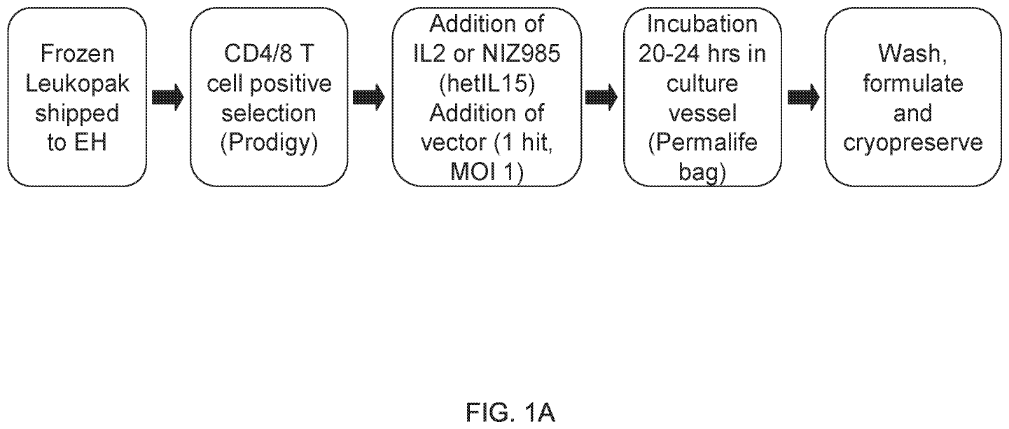

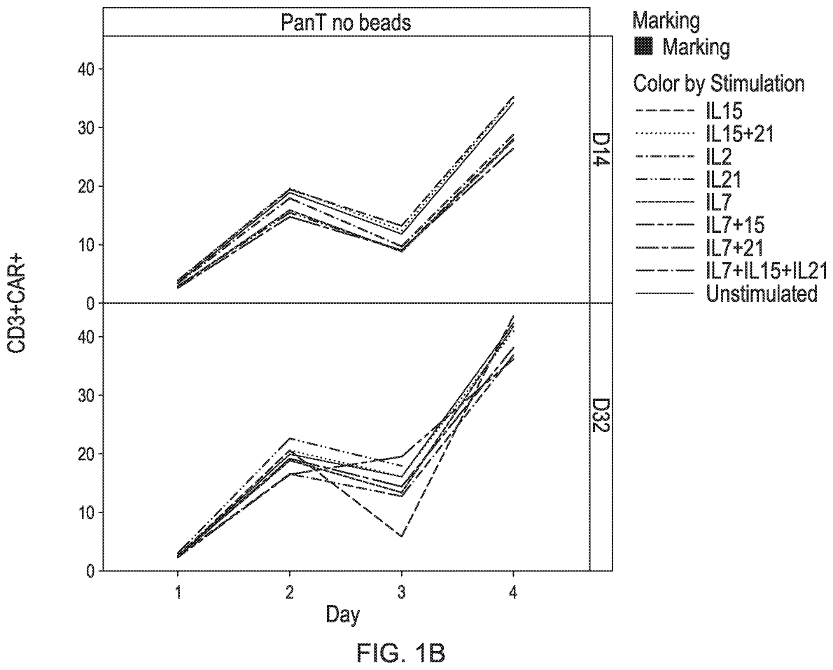

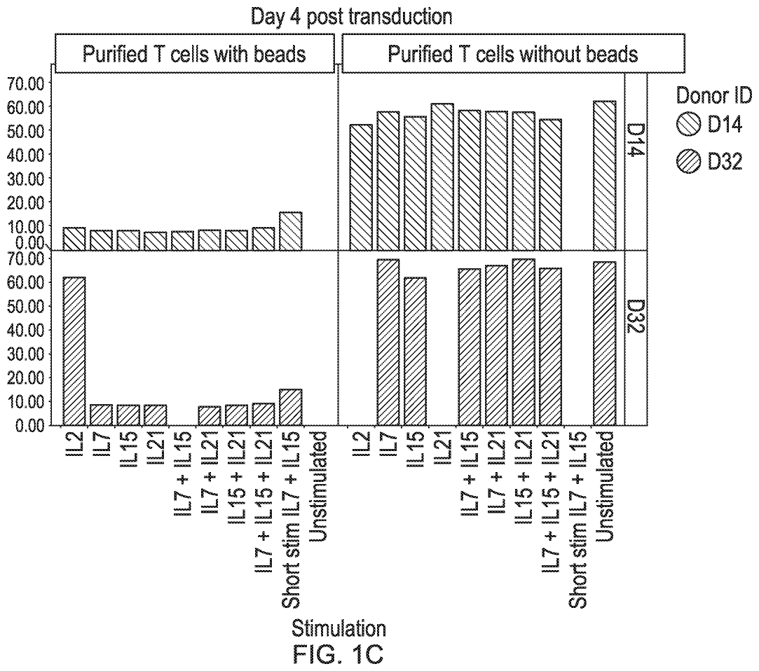

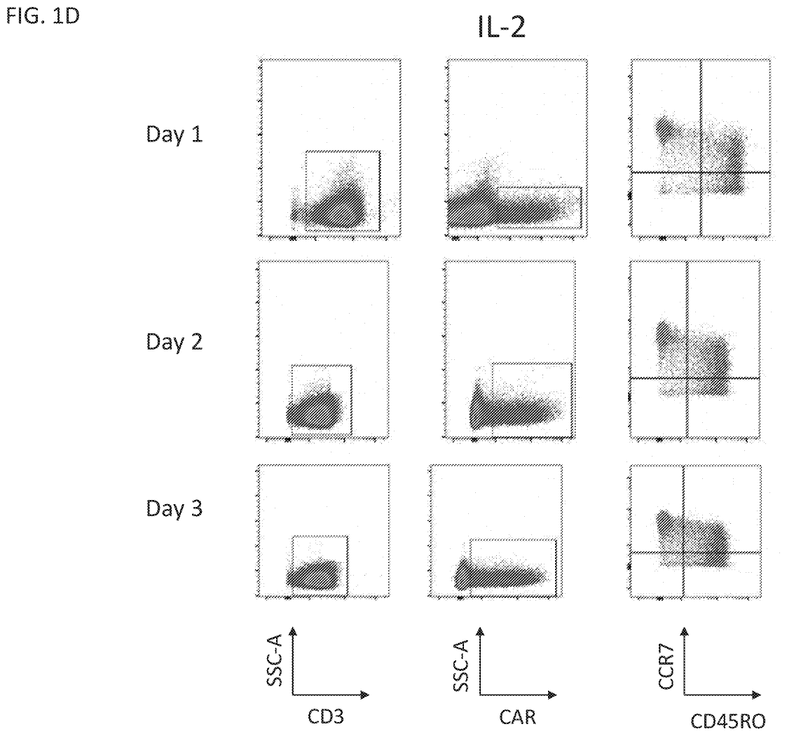

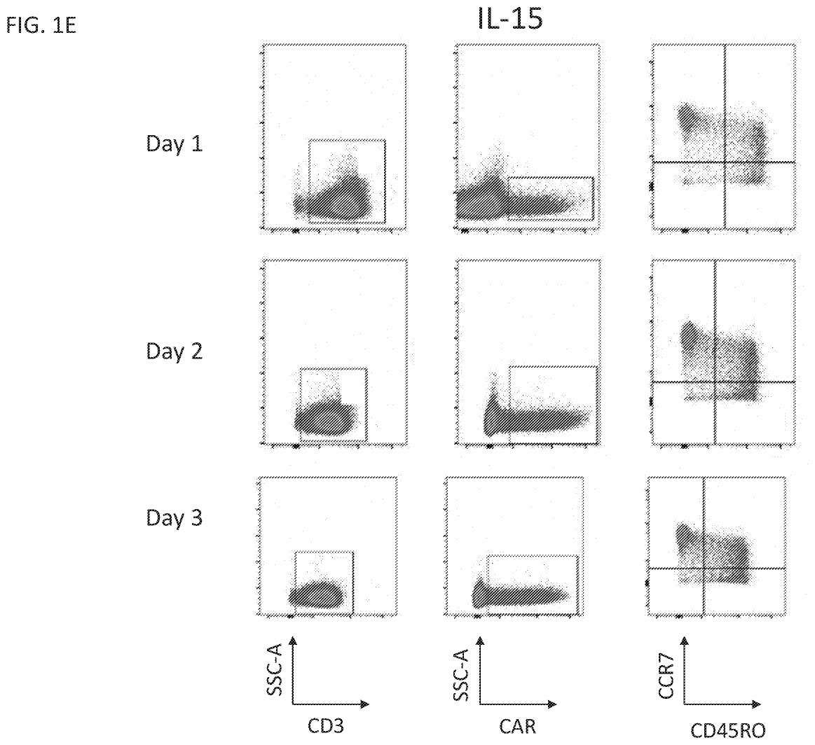

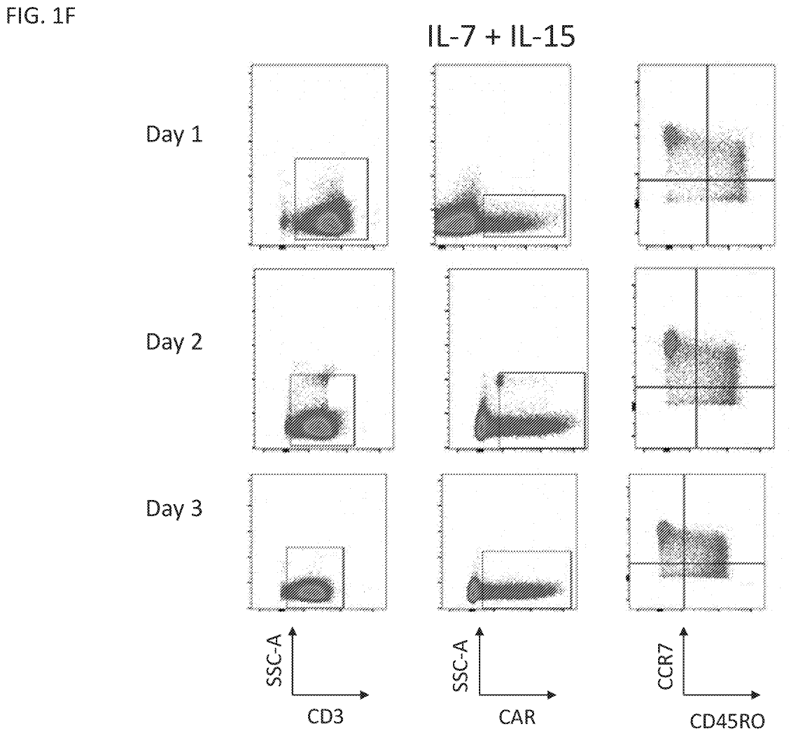

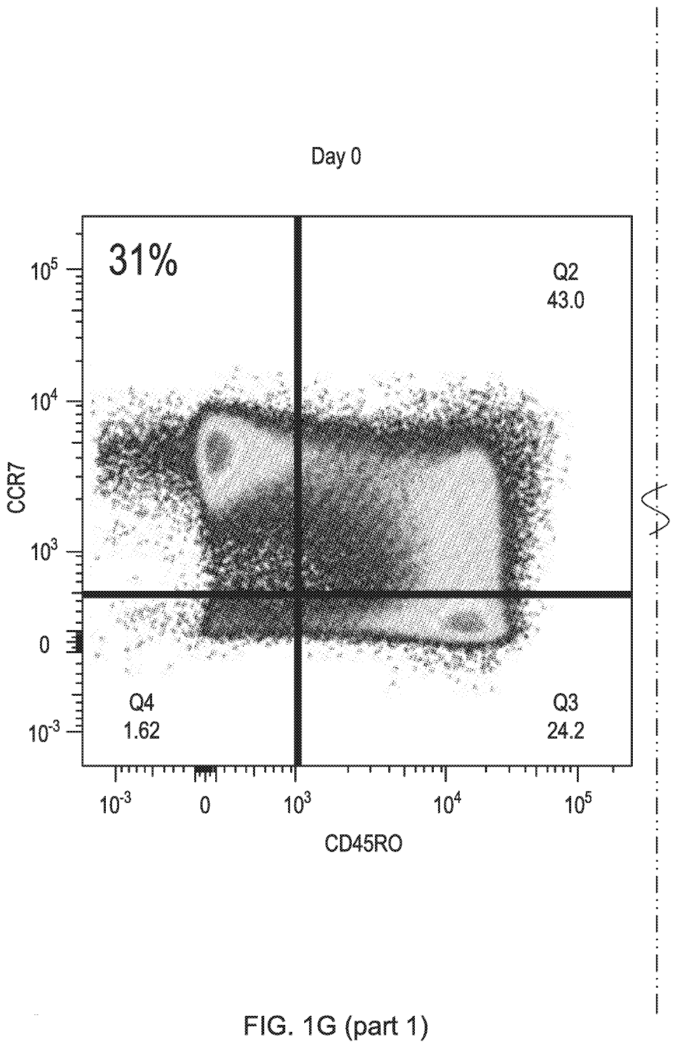

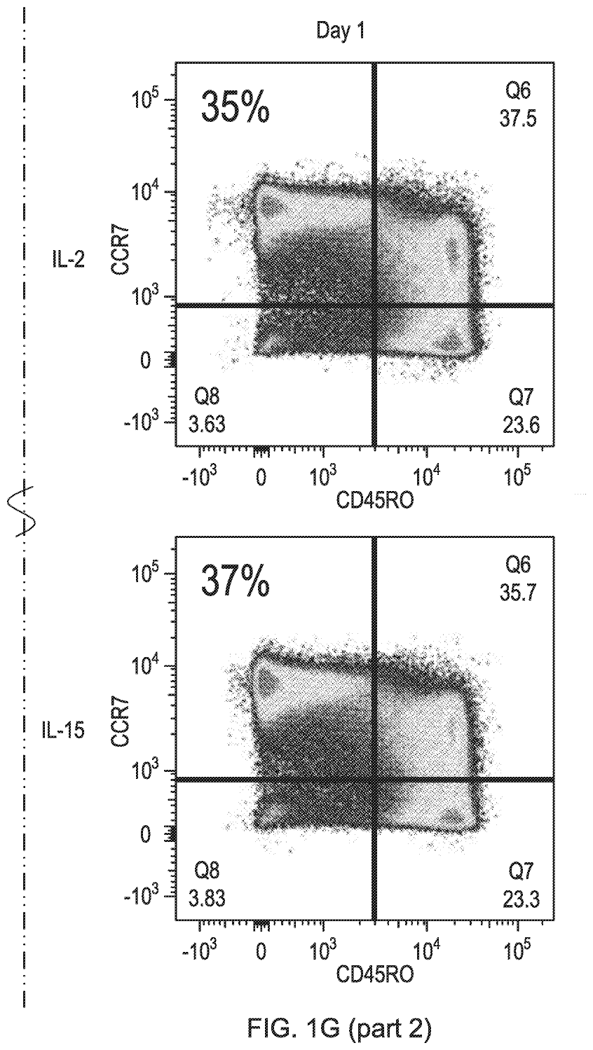

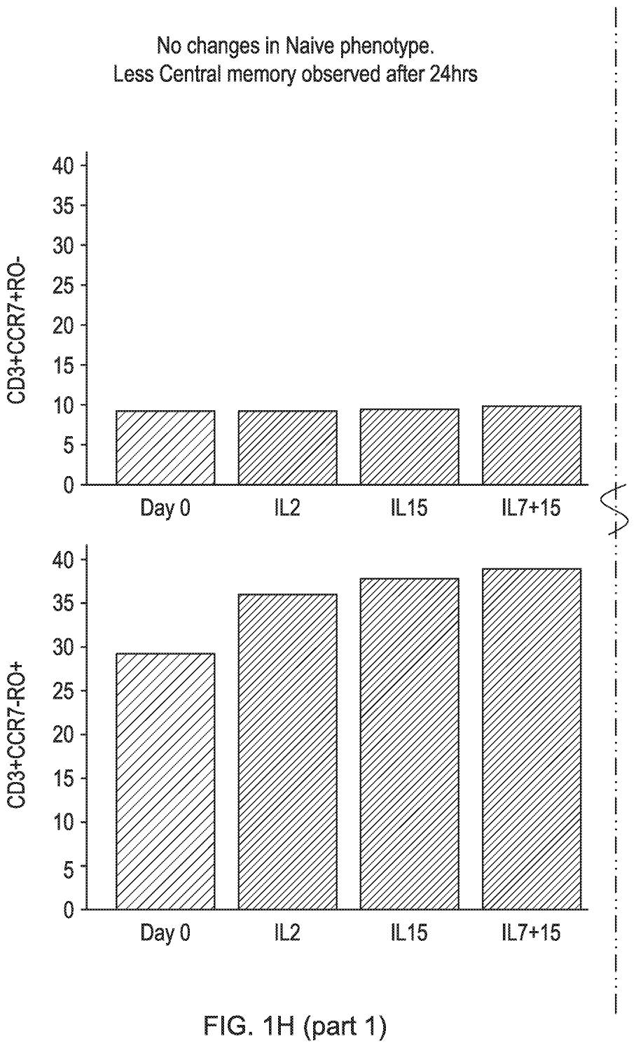

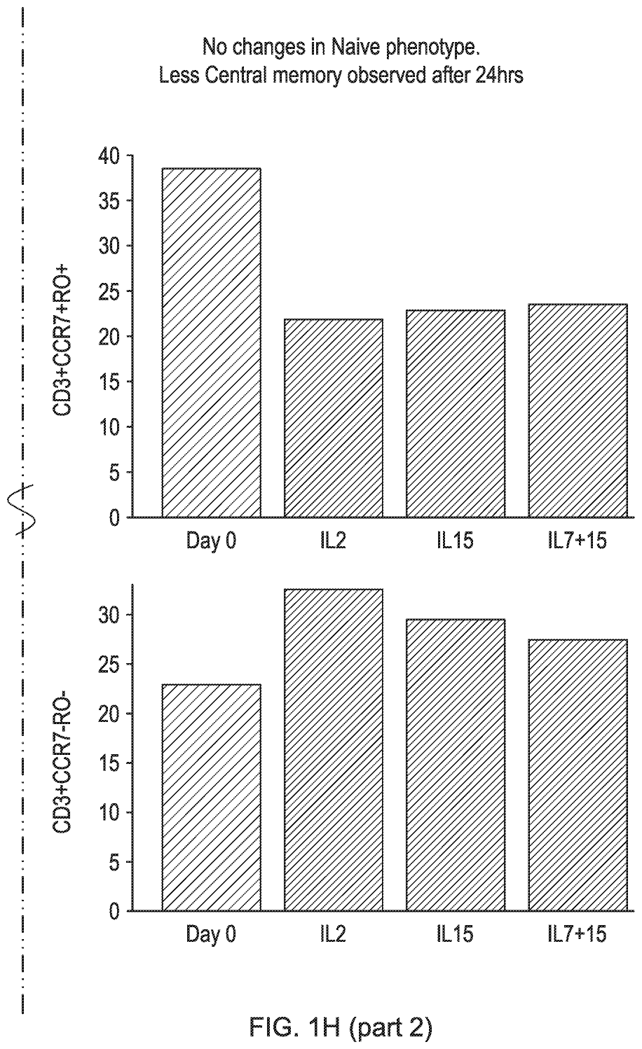

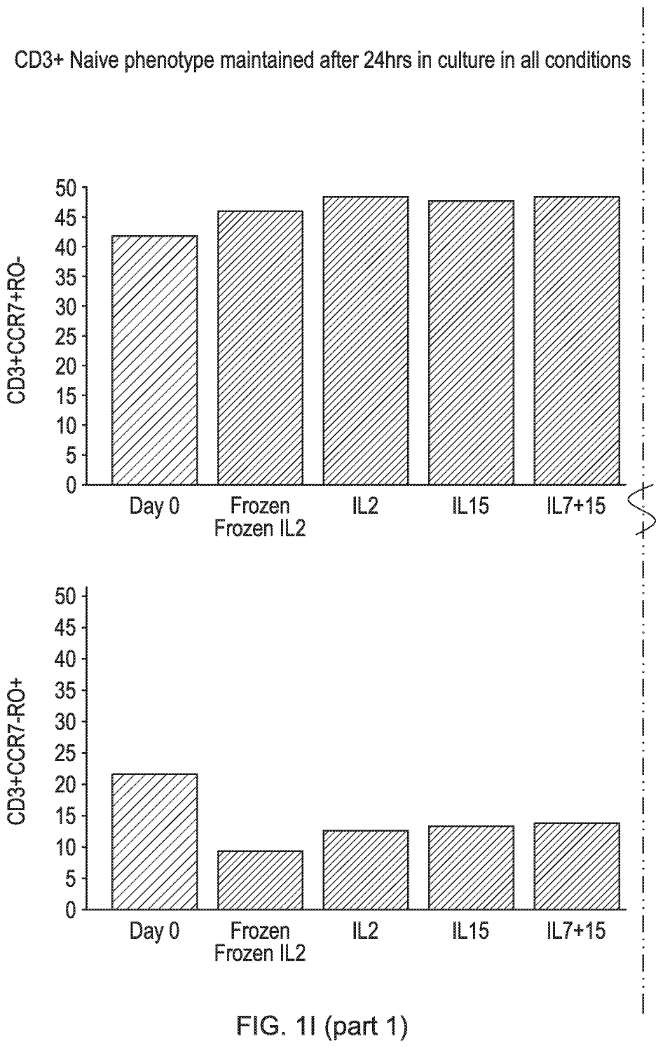

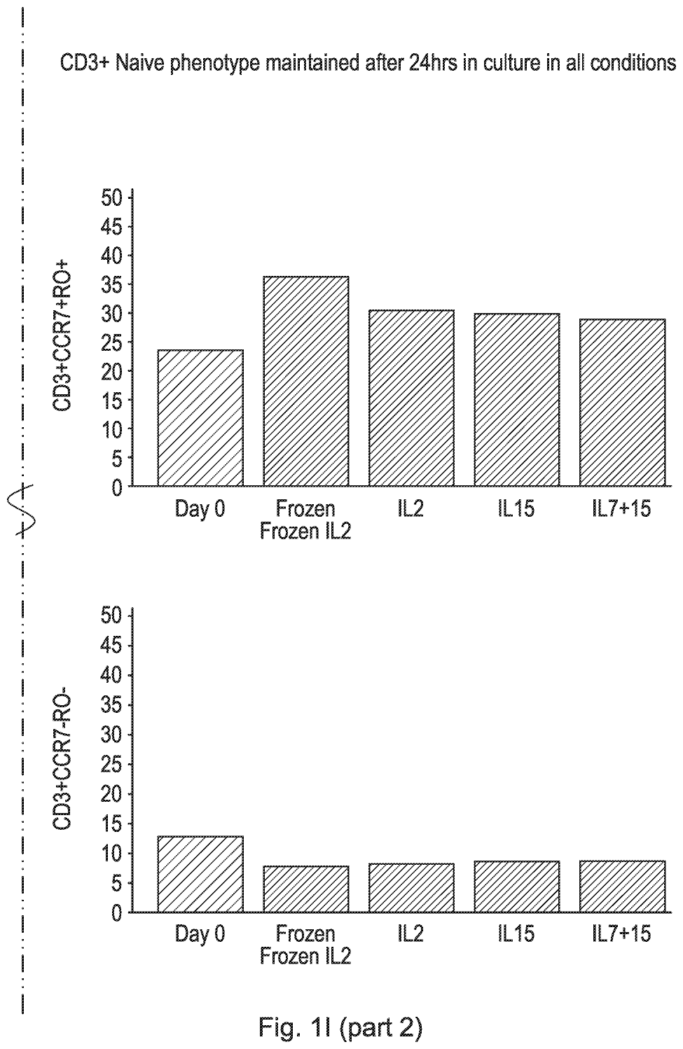

For clarity purposes, unless otherwise noted, classifying a cell or a population of cells as “not expressing,” or having an “absence of” or being “negative for” a particular marker may not necessarily mean an absolute absence of the marker. The skilled artisan can readily compare the cell against a positive and/or a negative control, and/or set a predetermined threshold, and classify the cell or population of cells as not expressing or being negative for the marker when the cell has an expression level below the predetermined threshold or a population of cells has an overall expression level below the predetermined threshold using conventional detection methods, e.g., using flow cytometry, for example, as described in the Examples herein. For example, representative gating strategies are shown in FIG. 1G. For example, CCR7 positive, CD45RO negative cells are shown in the top left quadrant in FIG. 1G.

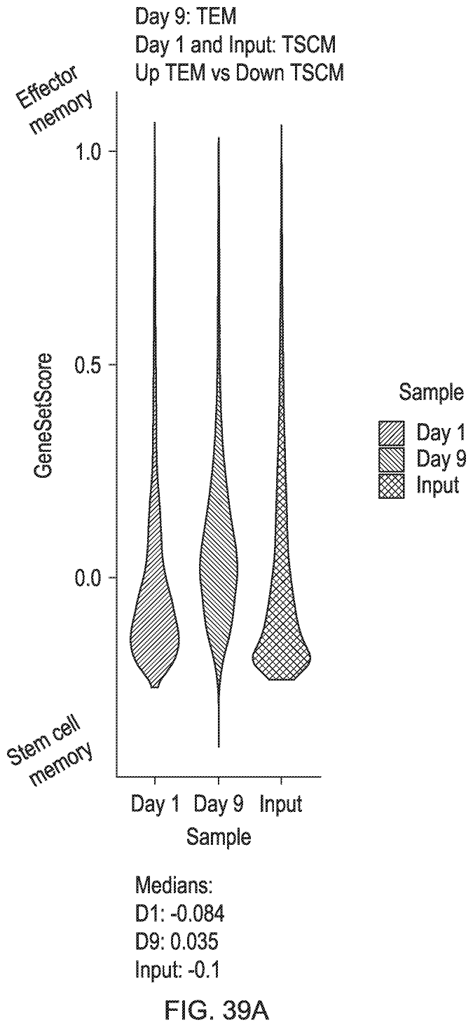

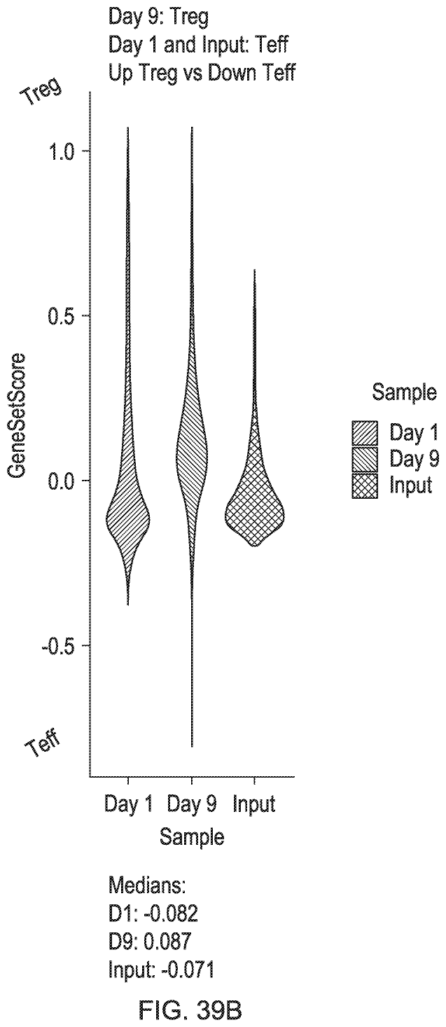

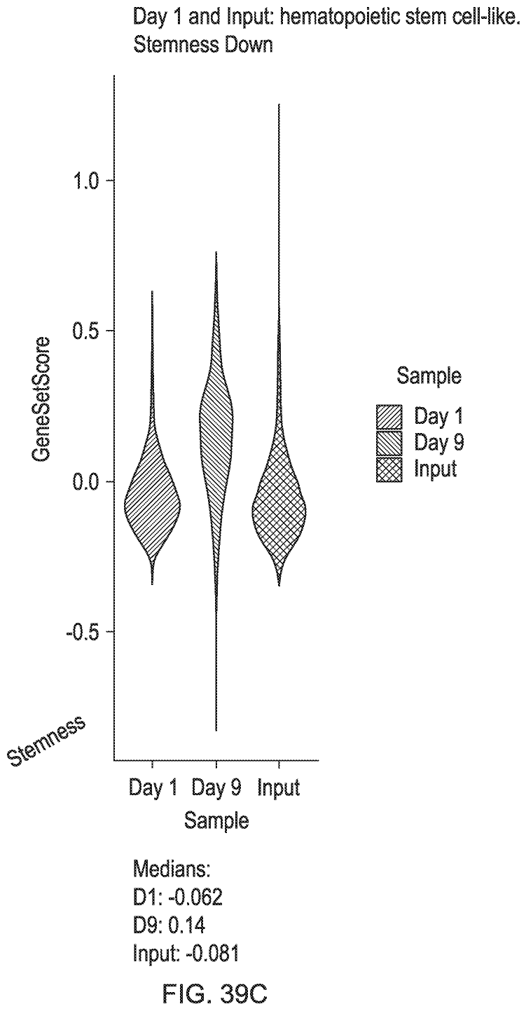

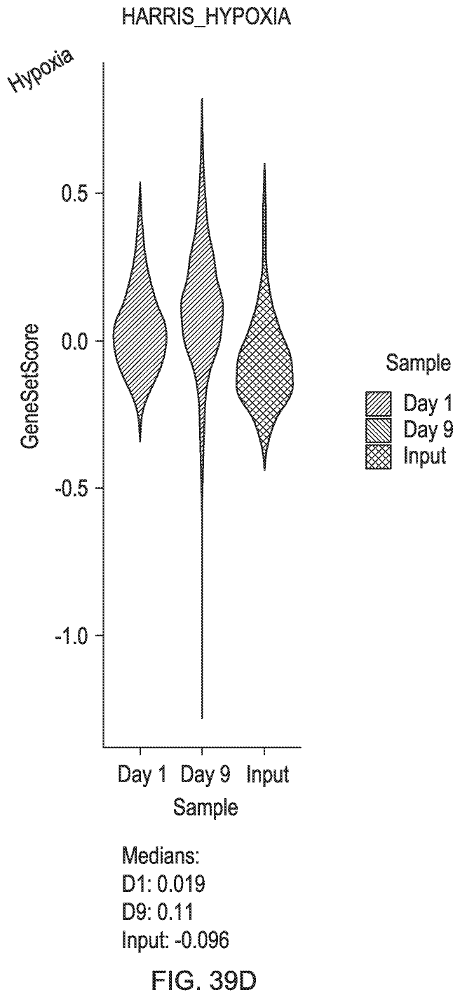

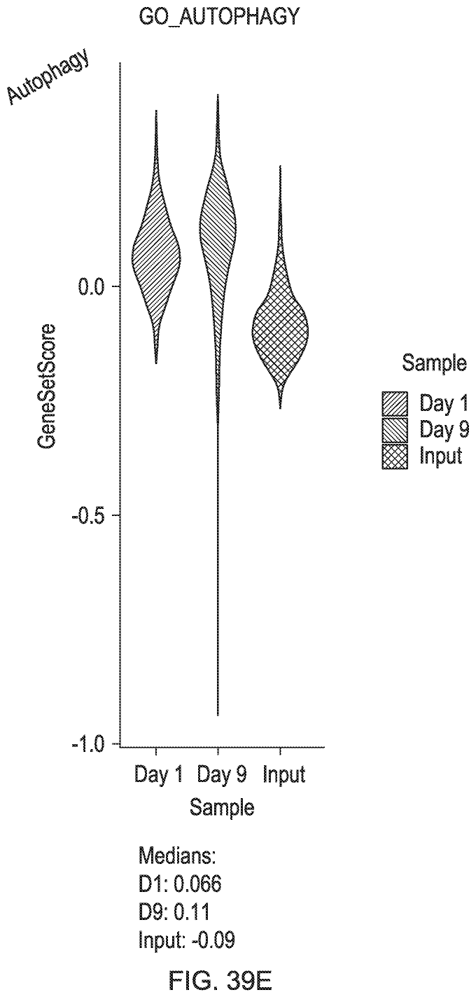

As used herein, the term “GeneSetScore (Up TEM vs. Down TSCM)” of a cell refers to a score that reflects the degree at which the cell shows an effector memory T cell (TEM) phenotype vs. a stem cell memory T cell (TSCM) phenotype. A higher GeneSetScore (Up TEM vs. Down TSCM) indicates an increasing TEM phenotype, whereas a lower GeneSetScore (Up TEM vs. Down TSCM) indicates an increasing TSCM phenotype. In some embodiments, the GeneSetScore (Up TEM vs. Down TSCM) is determined by measuring the expression of one or more genes that are up-regulated in TEM cells and/or down-regulated in TSCM cells, for example, one or more genes selected from the group consisting of MXRA7, CLIC1, NAT13, TBC1D2B, GLCCI1, DUSP10, APOBEC3D, CACNB3, ANXA2P2, TPRG1, EOMES, MATK, ARHGAP10, ADAM8, MAN1A1, SLFN12L, SH2D2A, EIF2C4, CD58, MYO1F, RAB27B, ERN1, NPC1, NBEAL2, APOBEC3G, SYTL2, SLC4A4, PIK3AP1, PTGDR, MAF, PLEKHA5, ADRB2, PLXND1, GNAO1, THBS1, PPP2R2B, CYTH3, KLRF1, FLJ16686, AUTS2, PTPRM, GNLY, and GFPT2. In some embodiments, the GeneSetScore (Up TEM vs. Down TSCM) is determined for each cell using RNA-seq, for example, single-cell RNA-seq (scRNA-seq), for example, as exemplified in Example 10 with respect to FIG. 39A. In some embodiments, the GeneSetScore (Up TEM vs. Down TSCM) is calculated by taking the mean log normalized gene expression value of all of the genes in the gene set.

As used herein, the term “GeneSetScore (Up Treg vs. Down Teff)” of a cell refers to a score that reflects the degree at which the cell shows a regulatory T cell (Treg) phenotype vs. an effector T cell (Teff) phenotype. A higher GeneSetScore (Up Treg vs. Down Teff) indicates an increasing Treg phenotype, whereas a lower GeneSetScore (Up Treg vs. Down Teff) indicates an increasing Teff phenotype. In some embodiments, the GeneSetScore (Up Treg vs. Down Teff) is determined by measuring the expression of one or more genes that are up-regulated in Treg cells and/or down-regulated in Teff cells, for example, one or more genes selected from the group consisting of C12orf75, SELPLG, SWAP70, RGS1, PRR11, SPATS2L, SPATS2L, TSHR, C14orf145, CASP8, SYT11, ACTN4, ANXA5, GLRX, HLA-DMB, PMCH, RAB11FIP1, IL32, FAM160B1, SHMT2, FRMD4B, CCR3, TNFRSF13B, NTNG2, CLDND1, BARD1, FCER1G, TYMS, ATP1B1, GJB6, FGL2, TK1, SLC2A8, CDKN2A, SKAP2, GPR55, CDCA7, S100A4, GDPD5, PMAIP1, ACOT9, CEP55, SGMS1, ADPRH, AKAP2, HDAC9, IKZF4, CARD17, VAV3, OBFC2A, ITGB1, CIITA, SETD7, HLA-DMA, CCR10, KIAA0101, SLC14A1, PTTG3P, DUSP10, FAM164A, PYHIN1, MYO1F, SLC1A4, MYBL2, PTTG1, RRM2, TP53INP1, CCR5, ST8SIA6, TOX, BFSP2, ITPRIPL1, NCAPH, HLA-DPB2, SYT4, NINJ2, FAM46C, CCR4, GBP5, C15orf53, LMCD1, MKI67, NUSAP1, PDE4A, E2F2, CD58, ARHGEF12, LOC100188949, FAS, HLA-DPB1, SELP, WEE1, HLA-DPA1, FCRL1, ICA1, CNTNAP1, OAS1, METTL7A, CCR6, HLA-DRB4, ANXA2P3, STAM, HLA-DQB2, LGALS1, ANXA2, PI16, DUSP4, LAYN, ANXA2P2, PTPLA, ANXA2P1, ZNF365, LAIR2, LOC541471, RASGRP4, BCAS1, UTS2, MIAT, PRDM1, SEMA3G, FAM129A, HPGD, NCF4, LGALS3, CEACAM4, JAKMIP1, TIGIT, HLA-DRA, IKZF2, HLA-DRB1, FANK1, RTKN2, TRIB1, FCRL3, and FOXP3. In some embodiments, the GeneSetScore (Up Treg vs. Down Teff) is determined using RNA-seq, for example, single-cell RNA-seq (scRNA-seq), for example, as exemplified in Example 10 with respect to FIG. 39B. In some embodiments, the GeneSetScore (Up Treg vs. Down Teff) is calculated by taking the mean log normalized gene expression value of all of the genes in the gene set.

As used herein, the term “GeneSetScore (Down stemness)” of a cell refers to a score that reflects the degree at which the cell shows a stemness phenotype. A lower GeneSetScore (Down stemness) indicates an increasing stemness phenotype. In some embodiments, the GeneSetScore (Down stemness) is determined by measuring the expression of one or more genes that are upregulated in a differentiating stem cell vs downregulated in a hematopoietic stem cell, for example, one or more genes selected from the group consisting of ACE, BATF, 30) CDK6, CHD2, ERCC2, HOXB4, MEOX1, SFRP1, SP7, SRF, TAL1, and XRCC5. In some embodiments, the GeneSetScore (Down stemness) is determined using RNA-seq, for example, single-cell RNA-seq (scRNA-seq), for example, as exemplified in Example 10 with respect to FIG. 39C. In some embodiments, the GeneSetScore (Down stemness) is calculated by taking the mean log normalized gene expression value of all of the genes in the gene set.

As used herein, the term “GeneSetScore (Up hypoxia)” of a cell refers to a score that reflects the degree at which the cell shows a hypoxia phenotype. A higher GeneSetScore (Up hypoxia) indicates an increasing hypoxia phenotype. In some embodiments, the GeneSetScore (Up hypoxia) is determined by measuring the expression of one or more genes that are up-regulated in cells undergoing hypoxia, for example, one or more genes selected from the group consisting of ABCB1, ACAT1, ADM, ADORA2B, AK2, AK3, ALDH1A1, ALDH1A3, ALDOA, ALDOC, ANGPT2, ANGPTL4, ANXA1, ANXA2, ANXA5, ARHGAP5, ARSE, ART1, BACE2, BATF3, BCL2L1, BCL2L2, BHLHE40, BHLHE41, BIK, BIRC2, BNIP3, BNIP3L, BPI, BTG1, C11orf2, C7orf68, CA12, CA9, CALD1, CCNG2, CCT6A, CD99, CDK1, CDKN1A, CDKN1B, CITED2, CLK1, CNOT7, COL4A5, COL5A1, COL5A2, COL5A3, CP, CTSD, CXCR4, D4S234E, DDIT3, DDIT4, 1-Dec, DKC1, DR1, EDN1, EDN2, EFNA1, EGF, EGR1, EIF4A3, ELF3, ELL2, ENG, ENO1, ENO3, ENPEP, EPO, ERRFI1, ETS1, F3, FABP5, FGF3, FKBP4, FLT1, FN1, FOS, FTL, GAPDH, GBE1, GLRX, GPI, GPRC5A, HAP1, HBP1, HDAC1, HDAC9, HERC3, HERPUD1, HGF, HIF1A, HK1, HK2, HLA-DQB1, HMOX1, HMOX2, HSPA5, HSPD1, HSPH1, HYOU1, ICAM1, ID2, IFI27, IGF2, IGFBP1, IGFBP2, IGFBP3, IGFBP5, IL6, IL8, INSIG1, IRF6, ITGA5, JUN, KDR, 20) KRT14, KRT18, KRT19, LDHA, LDHB, LEP, LGALS1, LONP1, LOX, LRP1, MAP4, MET, MIF, MMP13, MMP2, MMP7, MPI, MT1L, MTL3P, MUC1, MXI1, NDRG1, NFIL3, NFKB1, NFKB2, NOS1, NOS2, NOS2P1, NOS2P2, NOS3, NR3C1, NR4A1, NT5E, ODC1, P4HA1, P4HA2, PAICS, PDGFB, PDK3, PFKFB1, PFKFB3, PFKFB4, PFKL, PGAM1, PGF, PGK1, PGK2, PGM1, PIM1, PIM2, PKM2, PLAU, PLAUR, PLIN2, PLOD2, PNN, PNP, POLM, PPARA, PPAT, PROK1, PSMA3, PSMD9, PTGS1, PTGS2, QSOX1, RBPJ, RELA, RIOK3, RNASEL, RPL36A, RRP9, SATI, SERPINB2, SERPINE1, SGSM2, SIAH2, SIN3A, SIRPA, SLC16A1, SLC16A2, SLC20A1, SLC2A1, SLC2A3, SLC3A2, SLC6A10P, SLC6A16, SLC6A6, SLC6A8, SORL1, SPP1, SRSF6, SSSCA1, STC2, STRA13, SYT7, TBPL1, TCEAL1, TEK, TF, TFF3, TFRC, TGFA, TGFB1, TGFB3, TGFBI, TGM2, TH, THBS1, THBS2, TIMM17A, TNFAIP3, TP53, TPBG, TPD52, TPI1, TXN, TXNIP, UMPS, VEGFA, VEGFB, VEGFC, VIM, VPS11, and XRCC6. In some embodiments, the GeneSetScore (Up hypoxia) is determined using RNA-seq, for example, single-cell RNA-seq (scRNA-seq), for example, as exemplified in Example 10 with respect to FIG. 39D. In some embodiments, the GeneSetScore (Up hypoxia) is calculated by taking the mean log normalized gene expression value of all of the genes in the gene set.

As used herein, the term “GeneSetScore (Up autophagy)” of a cell refers to a score that reflects the degree at which the cell shows an autophagy phenotype. A higher GeneSetScore (Up autophagy) indicates an increasing autophagy phenotype. In some embodiments, the GeneSetScore (Up autophagy) is determined by measuring the expression of one or more genes that are up-regulated in cells undergoing autophagy, for example, one or more genes selected from the group consisting of ABL1, ACBD5, ACIN1, ACTRT1, ADAMTS7, AKR1E2, ALKBH5, ALPK1, AMBRA1, ANXA5, ANXA7, ARSB, ASB2, ATG10, ATG12, ATG13, ATG14, ATG16L1, ATG16L2, ATG2A, ATG2B, ATG3, ATG4A, ATG4B, ATG4C, ATG4D, ATG5, ATG7, ATG9A, ATG9B, ATP13A2, ATP1B1, ATPAF1-AS1, ATPIF1, BECN1, BECN1P1, BLOC1S1, BMP2KL, BNIP1, BNIP3, BOC, C11orf2, C11orf41, C12orf44, C12orf5, C14orf133, C1orf210, C5, C6orf106, C7orf59, C7orf68, C8orf59, C9orf72, CA7, CALCB, CALCOCO2, CAPS, CCDC36, CD163L1, CD93, CDC37, CDKN2A, CHAF1B, CHMP2A, CHMP2B, CHMP3, CHMP4A, CHMP4B, CHMP4C, CHMP6, CHST3, CISD2, CLDN7, CLEC16A, CLN3, CLVS1, COX8A, CPA3, CRNKL1, CSPG5, CTSA, CTSB, CTSD, CXCR7, DAP, DKKL1, DNAAF2, DPF3, DRAM1, DRAM2, DYNLL1, DYNLL2, DZANK1, EI24, EIF2S1, EPG5, EPM2A, FABP1, FAM125A, FAM131B, FAM134B, FAM13B, FAM176A, FAM176B, FAM48A, FANCC, FANCF, FANCL, FBXO7, FCGR3B, FGF14, FGF7, FGFBP1, FIS1, FNBP1L, FOXO1, FUNDC1, FUNDC2, FXR2, GABARAP, GABARAPL1, GABARAPL2, GABARAPL3, GABRA5, GDF5, GMIP, HAP1, HAPLN1, HBXIP, HCAR1, HDAC6, HGS, HIST1H3A, HIST1H3B, HIST1H3C, HIST1H3D, HIST1H3E, HIST1H3F, HIST1H3G, HIST1H3H, HIST1H3I, HIST1H3J, HK2, HMGB1, HPR, HSF2BP, HSP90AA1, HSPA8, IFI16, IPPK, IRGM, IST1, ITGB4, ITPKC, KCNK3, KCNQ1, KIAA0226, KIAA1324, KRCC1, KRT15, KRT73, LAMP1, LAMP2, LAMTOR1, LAMTOR2, LAMTOR3, LARP1B, LENG9, LGALS8, LIX1, LIX1L, LMCD1, LRRK2, LRSAM1, LSM4, MAP1A, MAP1LC3A, MAP1LC3B, MAP1LC3B2, MAP1LC3C, MAP1S, MAP2K1, MAP3K12, MARK2, MBD5, MDH1, MEX3C, MFN1, MFN2, MLST8, MRPS10, MRPS2, MSTN, MTERFD1, MTMR14, MTMR3, MTOR, MTSS1, MYH11, MYLK, MYOM1, NBR1, NDUFB9, NEFM, NHLRC1, NME2, NPC1, NR2C2, NRBF2, NTHL1, NUP93, OBSCN, OPTN, P2RX5, PACS2, PARK2, PARK7, PDK1, PDK4, PEX13, PEX3, PFKP, PGK2, PHF23, PHYHIP, PI4K2A, PIK3C3, PIK3CA, PIK3CB, PIK3R4, PINK1, PLEKHM1, PLOD2, PNPO, PPARGC1A, PPY, PRKAA1, PRKAA2, PRKAB1, PRKAB2, PRKAG1, PRKAG2, PRKAG3, PRKD2, PRKG1, PSEN1, PTPN22, RAB12, RAB1A, RAB1B, RAB23, RAB24, RAB33B, RAB39, RAB7A, RB1CC1, RBM18, REEP2, REP15, RFWD3, RGS19, RHEB, RIMS3, RNF185, RNF41, RPS27A, RPTOR, RRAGA, RRAGB, RRAGC, RRAGD, S100A8, S100A9, SCN1A, SERPINB10, SESN2, SFRP4, SH3GLB1, SIRT2, SLC1A3, SLC1A4, SLC22A3, SLC25A19, SLC35B3, SLC35C1, SLC37A4, SLC6A1, SLCO1A2, SMURF1, SNAP29, SNAPIN, SNF8, SNRPB, SNRPB2, SNRPD1, SNRPF, SNTG1, SNX14, SPATA18, SQSTM1, SRPX, STAM, STAM2, STAT2, STBD1, STK11, STK32A, STOM, STX12, STX17, SUPT3H, TBC1D17, TBC1D25, TBC1D5, TCIRG1, TEAD4, TECPR1, TECPR2, TFEB, TM9SF1, TMBIM6, TMEM203, TMEM208, TMEM39A, TMEM39B, TMEM59, TMEM74, TMEM93, TNIK, TOLLIP, TOMM20, TOMM22, TOMM40, TOMM5, TOMM6, TOMM7, TOMM70A, TP53INP1, TP53INP2, TRAPPC8, TREM1, TRIM17, TRIM5, TSG101, TXLNA, UBA52, UBB, UBC, UBQLN1, UBQLN2, UBQLN4, ULK1, ULK2, ULK3, USP10, USP13, USP30, UVRAG, VAMP7, VAMP8, VDAC1, VMP1, VPS11, VPS16, VPS18, VPS25, VPS28, VPS33A, VPS33B, VPS36, VPS37A, VPS37B, VPS37C, VPS37D, VPS39, VPS41, VPS4A, VPS4B, VTA1, VTI1A, VTI1B, WDFY3, WDR45, WDR45L, WIPI1, WIPI2, XBP1, YIPF1, ZCCHC17, ZFYVE1, ZKSCAN3, ZNF189, ZNF593, and ZNF681. In some embodiments, the GeneSetScore (Up autophagy) is determined using RNA-seq, for example, single-cell RNA-seq (scRNA-seq), for example, as exemplified in Example 10 with respect to FIG. 39E. In some embodiments, the GeneSetScore (Up autophagy) is calculated by taking the mean log normalized gene expression value of all of the genes in the gene set.

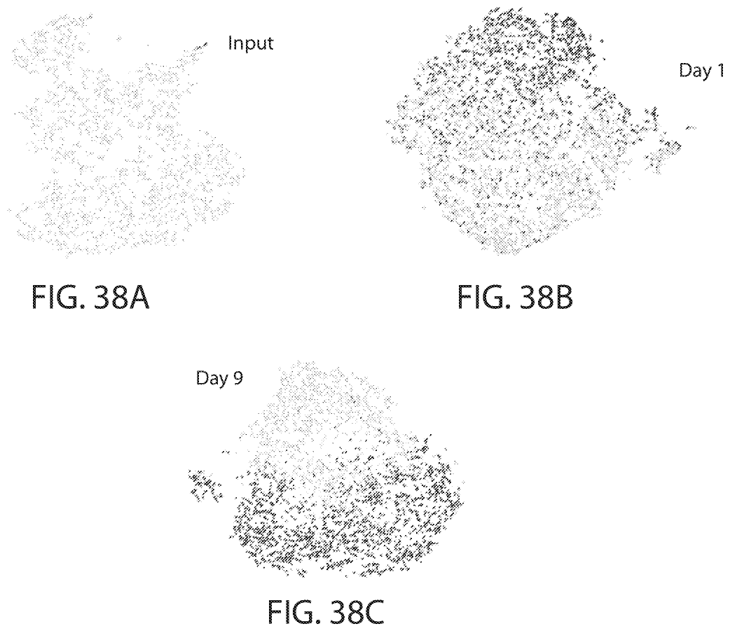

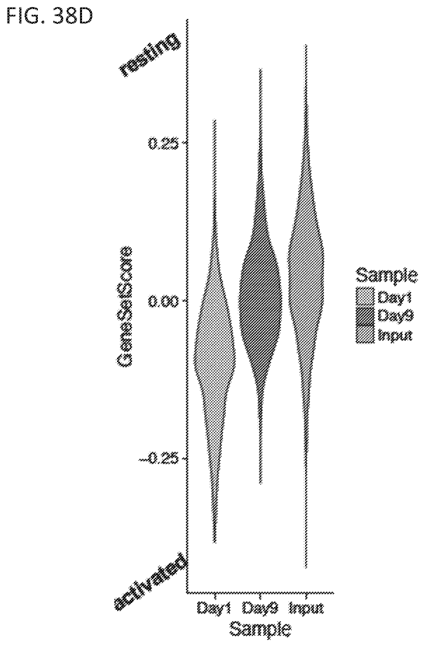

As used herein, the term “GeneSetScore (Up resting vs. Down activated)” of a cell refers to a score that reflects the degree at which the cell shows a resting T cell phenotype vs. an activated T cell phenotype. A higher GeneSetScore (Up resting vs. Down activated) indicates an increasing resting T cell phenotype, whereas a lower GeneSetScore (Up resting vs. Down activated) indicates an increasing activated T cell phenotype. In some embodiments, the GeneSetScore (Up resting vs. Down activated) is determined by measuring the expression of one or more genes that are up-regulated in resting T cells and/or down-regulated in activated T cells, for example, one or more genes selected from the group consisting of ABCA7, ABCF3, ACAP2, AMT, ANKH, ATF7IP2, ATG14, ATP1A1, ATXN7, ATXN7L3B, BCL7A, BEX4, BSDC1, BTG1, BTG2, BTN3A1, C11orf21, C19orf22, C21orf2, CAMK2G, CARS2, CCNL2, CD248, CD5, CD55, CEP164, CHKB, CLK1, CLK4, CTSL1, DBP, DCUN1D2, DENND1C, DGKD, DLG1, DUSP1, EAPP, ECE1, ECHDC2, ERBB2IP, FAM117A, FAM134B, FAM134C, FAM169A, FAM190B, FAU, FLJ10038, FOXJ2, FOXJ3, FOXL1, FOXO1, FXYD5, FYB, HLA-E, HSPA1L, HYAL2, ICAM2, IFIT5, IFITM1, IKBKB, IQSEC1, IRS4, KIAA0664L3, KIAA0748, KLF3, KLF9, KRT18, LEF1, LINC00342, LIPA, LIPT1, LLGL2, LMBR1L, LPAR2, LTBP3, LYPD3, LZTFL1, MANBA, MAP2K6, MAP3K1, MARCH8, MAU2, MGEA5, MMP8, MPO, MSL1, MSL3, MYH3, MYLIP, NAGPA, NDST2, NISCH, NKTR, NLRP1, NOSIP, NPIP, NUMA1, PAIP2B, PAPD7, PBXIP1, PCIF1, PI4KA, PLCL2, PLEKHA1, PLEKHF2, PNISR, PPFIBP2, PRKCA, PRKCZ, PRKD3, PRMT2, PTP4A3, PXN, RASA2, RASA3, RASGRP2, RBM38, REPIN1, RNF38, RNF44, ROR1, RPL30, RPL32, RPLP1, RPS20, RPS24, RPS27, RPS6, RPS9, RXRA, RYK, SCAND2, SEMA4C, SETD1B, SETD6, SETX, SF3B1, SH2B1, SLC2A4RG, SLC35E2B, SLC46A3, SMAGP, SMARCE1, SMPD1, SNPH, SP140L, SPATA6, SPG7, SREK1IP1, SRSF5, STAT5B, SVIL, SYF2, SYNJ2BP, TAF1C, TBC1D4, TCF20, TECTA, TES, TMEM127, TMEM159, TMEM30B, TMEM66, TMEM8B, TP53TG1, TPCN1, TRIM22, TRIM44, TSC1, TSC22D1, TSC22D3, TSPYL2, TTC9, TTN, UBE2G2, USP33, USP34, VAMP1, VILL, VIPR1, VPS13C, ZBED5, ZBTB25, ZBTB40, ZC3H3, ZFP161, ZFP36L1, ZFP36L2, ZHX2, ZMYM5, ZNF136, ZNF148, ZNF318, ZNF350, ZNF512B, ZNF609, ZNF652, ZNF83, ZNF862, and ZNF91. In some embodiments, the GeneSetScore (Up resting vs. Down activated) is determined using RNA-seq, for example, single-cell RNA-seq (scRNA-seq), for example, as exemplified in Example 10 with respect to FIG. 38D. In some embodiments, the GeneSetScore (Up resting vs. Down activated) is calculated by taking the mean log normalized gene expression value of all of the genes in the gene set.

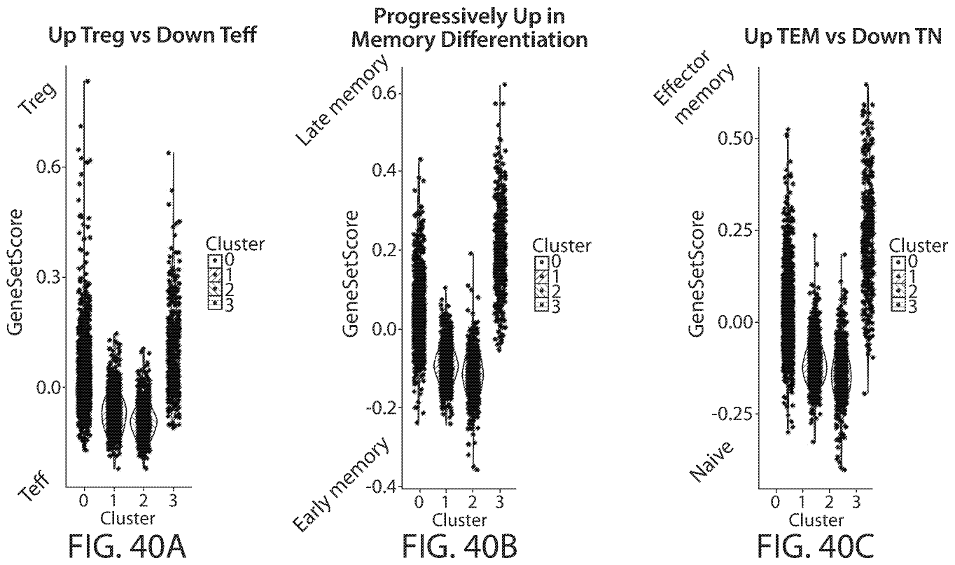

As used herein, the term “GeneSetScore (Progressively up in memory differentiation)” of a cell refers to a score that reflects the stage of the cell in memory differentiation. A higher GeneSetScore (Progressively up in memory differentiation) indicates an increasing late memory T cell phenotype, whereas a lower GeneSetScore (Progressively up in memory differentiation) indicates an increasing early memory T cell phenotype. In some embodiments, the GeneSetScore (Up autophagy) is determined by measuring the expression of one or more genes that are up-regulated during memory differentiation, for example, one or more genes selected from the group consisting of MTCH2, RAB6C, KIAA0195, SETD2, C2orf24, NRD1, GNA13, COPA, SELT, TNIP1, CBFA2T2, LRP10, PRKC1, BRE, ANKS1A, PNPLA6, ARL6IP1, WDFY1, MAPK1, GPR153, SHKBP1, MAP1LC3B2, PIP4K2A, HCN3, GTPBP1, TLN1, C4orf34, KIF3B, TCIRG1, PPP3CA, ATG4D, TYMP, TRAF6, C17orf76, WIPF1, FAM108A1, MYL6, NRM, SPCS2, GGT3P, GALK1, CLIP4, ARL4C, YWHAQ, LPCAT4, ATG2A, IDS, TBC1D5, DMPK, ST6GALNAC6, REEP5, ABHD6, KIAA0247, EMB, TSEN54, SPIRE2, PIWIL4, ZSCAN22, ICAM1, CHD9, LPIN2, SETD8, ZC3H12A, ULBP3, IL15RA, HLA-DQB2, LCP1, CHP, RUNX3, TMEM43, REEP4, MEF2D, ABL1, TMEM39A, PCBP4, PLCD1, CHST12, RASGRP1, C1orf58, C11orf63, C6orf129, FHOD1, DKFZp434F142, PIK3CG, ITPR3, BTG3, C4orf50, CNNM3, IFI16, AK1, CDK2AP1, REL, BCL2L1, MVD, TTC39C, PLEKHA2, FKBP11, EML4, FANCA, CDCA4, FUCA2, MFSD10, TBCD, CAPN2, IQGAP1, CHST11, PIK3R1, MYO5A, KIR2DL3, DLG3, MXD4, RALGDS, S1PR5, WSB2, CCR3, TIPARP, SP140, CD151, SOX13, KRTAP5-2, NF1, PEA15, PARP8, RNF166, UEVLD, LIMK1, CACNB1, TMX4, SLC6A6, LBA1, SV2A, LLGL2, IRF1, PPP2R5C, CD99, RAPGEF1, PPP4R1, OSBPL7, FOXP4, SLA2, TBC1D2B, ST7, JAZF1, GGA2, PI4K2A, CD68, LPGAT1, STX11, ZAK, FAM160B1, RORA, C8orf80, APOBEC3F, TGFBI, DNAJC1, GPR114, LRP8, CD69, CMIP, NAT13, TGFB1, FLJ00049, ANTXR2, NR4A3, IL12RB1, NTNG2, RDX, MLLT4, GPRIN3, ADCY9, CD300A, SCD5, ABI3, PTPN22, LGALS1, SYTL3, BMPR1A, TBK1, PMAIP1, RASGEF1A, GCNT1, GABARAPL1, STOM, CALHM2, ABCA2, PPP1R16B, SYNE2, PAM, C12orf75, CLCF1, MXRA7, APOBEC3C, CLSTN3, ACOT9, HIP1, LAG3, TNFAIP3, DCBLD1, KLF6, CACNB3, RNF19A, RAB27A, FADS3, DLG5, APOBEC3D, TNFRSF1B, ACTN4, TBKBP1, ATXN1, ARAP2, ARHGEF12, FAM53B, MAN1A1, FAM38A, PLXNC1, GRLF1, SRGN, HLA-DRB5, B4GALT5, WIPI1, PTPRJ, SLFN11, DUSP2, ANXA5, AHNAK, NEO1, CLIC1, EIF2C4, MAP3K5, IL2RB, PLEKHG1, MYO6, GTDC1, EDARADD, GALM, TARP, ADAM8, MSC, HNRPLL, SYT11, ATP2B4, NHSL2, MATK, ARHGAP18, SLFN12L, SPATS2L, RAB27B, PIK3R3, TP53INP1, MBOAT1, GYG1, KATNAL1, FAM46C, ZC3HAV1L, ANXA2P2, CTNNA1, NPC1, C3AR1, CRIM1, SH2D2A, ERN1, YPEL1, TBX21, SLC1A4, FASLG, PHACTR2, GALNT3, ADRB2, PIK3AP1, TLR3, PLEKHA5, DUSP10, GNAO1, PTGDR, FRMD4B, ANXA2, EOMES, CADM1, MAF, TPRG1, NBEAL2, PPP2R2B, PELO, SLC4A4, KLRF1, FOSL2, RGS2, TGFBR3, PRF1, MYO1F, GAB3, C17orf66, MICAL2, CYTH3, TOX, HLA-DRA, SYNE1, WEE1, PYHIN1, F2R, PLD1, THBS1, CD58, FAS, NETO2, CXCR6, ST6GALNAC2, DUSP4, AUTS2, C1orf21, KLRG1, TNIP3, GZMA, PRR5L, PRDM1, ST8SIA6, PLXND1, PTPRM, GFPT2, MYBL1, SLAMF7, FLJ16686, GNLY, ZEB2, CST7, IL18RAP, CCL5, KLRD1, and KLRB1. In some embodiments, the GeneSetScore (Progressively up in memory differentiation) is determined using RNA-seq, for example, single-cell RNA-seq (scRNA-seq), for example, as exemplified in Example 10 with respect to FIG. 40B. In some embodiments, the GeneSetScore (Progressively up in memory differentiation) is calculated by taking the mean log normalized gene expression value of all of the genes in the gene set.

As used herein, the term “GeneSetScore (Up TEM vs. Down TN)” of a cell refers to a score that reflects the degree at which the cell shows an effector memory T cell (TEM) phenotype vs. a naïve T cell (TN) phenotype. A higher GeneSetScore (Up TEM vs. Down TN) indicates an increasing TEM phenotype, whereas a lower GeneSetScore (Up TEM vs. Down TN) indicates an increasing TN phenotype. In some embodiments, the GeneSetScore (Up TEM vs. Down TN) is determined by measuring the expression of one or more genes that are up-regulated in TEM cells and/or down-regulated in TN cells, for example, one or more genes selected from the group consisting of MYO5A, MXD4, STK3, S1PR5, GLCCI1, CCR3, SOX13, KRTAP5-2, PEA15, PARP8, RNF166, UEVLD, LIMK1, SLC6A6, SV2A, KPNA2, OSBPL7, ST7, GGA2, PI4K2A, CD68, ZAK, RORA, TGFBI, DNAJC1, JOSD1, ZFYVE28, LRP8, OSBPL3, CMIP, NAT13, TGFB1, ANTXR2, NR4A3, RDX, ADCY9, CHN1, CD300A, SCD5, PTPN22, LGALS1, RASGEF1A, GCNT1, GLUL, ABCA2, CLDND1, PAM, CLCF1, MXRA7, CLSTN3, ACOT9, METRNL, BMPR1A, LRIG1, APOBEC3G, CACNB3, RNF19A, RAB27A, FADS3, ACTN4, TBKBP1, FAM53B, MAN1A1, FAM38A, GRLF1, B4GALT5, WIPI1, DUSP2, ANXA5, AHNAK, CLIC1, MAP3K5, ST8SIA1, TARP, ADAM8, MATK, SLFN12L, PIK3R3, FAM46C, ANXA2P2, CTNNA1, NPC1, SH2D2A, ERN1, YPEL1, TBX21, STOM, PHACTR2, GBP5, ADRB2, PIK3AP1, DUSP10, PTGDR, EOMES, MAF, TPRG1, NBEAL2, NCAPH, SLC4A4, FOSL2, RGS2, TGFBR3, MYO1F, C17orf66, CYTH3, WEE1, PYHIN1, F2R, THBS1, CD58, AUTS2, FAM129A, TNIP3, GZMA, PRR5L, PRDM1, PLXND1, PTPRM, GFPT2, MYBL1, SLAMF7, ZEB2, CST7, CCL5, GZMK, and KLRB1. In some embodiments, the GeneSetScore (Up TEM vs. Down TN) is determined using RNA-seq, for example, single-cell RNA-seq (scRNA-seq), for example, as exemplified in Example 10 with respect to FIG. 40C. In some embodiments, the GeneSetScore (Up TEM vs. Down TN) is calculated by taking the mean log normalized gene expression value of all of the genes in the gene set.

In the context of GeneSetScore values (e.g., median GeneSetScore values), when a positive GeneSetScore is reduced by 100%, the value becomes 0. When a negative GeneSetScore is increased by 100%, the value becomes 0. For example, in FIG. 39A, the median GeneSetScore of the Day 1 sample is-0.084; the median GeneSetScore of the Day 9 sample is 0.035; and the median GeneSetScore of the input sample is-0.1. In FIG. 39A, increasing the median GeneSetScore of the input sample by 100% leads to a GeneSetScore value of 0; and increasing the median GeneSetScore of the input sample by 200% leads to a GeneSetScore value of 0.1. In FIG. 39A, decreasing the median GeneSetScore of the Day 9 sample by 100% leads to a GeneSetScore value of 0; and decreasing the median GeneSetScore of the Day 9 sample by 200% leads to a GeneSetScore value of −0.035.

As used herein, the term “bead” refers to a discrete particle with a solid surface, ranging in size from approximately 0.1 μm to several millimeters in diameter. Beads may be spherical (for example, microspheres) or have an irregular shape. Beads may comprise a variety of materials including, but not limited to, paramagnetic materials, ceramic, plastic, glass, polystyrene, methylstyrene, acrylic polymers, titanium, latex, Sepharose™, cellulose, nylon and the like. In some embodiments, the beads are relatively uniform, about 4.5 μm in diameter, spherical, superparamagnetic polystyrene beads, for example, coated, for example, covalently coupled, with a mixture of antibodies against CD3 (for example, CD3 epsilon) and CD28. In some embodiments, the beads are Dynabeads®. In some embodiments, both anti-CD3 and anti-CD28 antibodies are coupled to the same bead, mimicking stimulation of T cells by antigen presenting cells. The property of Dynabeads® and the use of Dynabeads® for cell isolation and expansion are well known in the art, for example, see, Neurauter et al., Cell isolation and expansion using Dynabeads, Adv Biochem Eng Biotechnol. 2007; 10⁶:41-73, herein incorporated by reference in its entirety.

As used herein, the term “nanomatrix” refers to a nanostructure comprising a matrix of mobile polymer chains. The nanomatrix is 1 to 500 nm, for example, 10 to 200 nm, in size. In some embodiments, the matrix of mobile polymer chains is attached to one or more agonists which provide activation signals to T cells, for example, agonist anti-CD3 and/or anti-CD28 antibodies. In some embodiments, the nanomatrix comprises a colloidal polymeric nanomatrix attached, for example, covalently attached, to an agonist of one or more stimulatory molecules and/or an agonist of one or more costimulatory molecules. In some embodiments, the agonist of one or more stimulatory molecules is a CD3 agonist (for example, an anti-CD3 agonistic antibody). In some embodiments, the agonist of one or more costimulatory molecules is a CD28 agonist (for example, an anti-CD28 agonistic antibody). In some embodiments, the nanomatrix is characterized by the absence of a solid surface, for example, as the attachment point for the agonists, such as anti-CD3 and/or anti-CD28 antibodies. In some embodiments, the nanomatrix is the nanomatrix disclosed in WO2014/048920A1 or as given in the MACS® GMP T Cell TransAct™ kit from Miltenyi Biotcc GmbH, herein incorporated by reference in their entirety. MACS® GMP T Cell TransAct™ consists of a colloidal polymeric nanomatrix covalently attached to humanized recombinant agonist antibodies against human CD3 and CD28.

Various embodiments of the compositions and methods herein are described in further detail below. Additional definitions are set out throughout the specification.

### DESCRIPTION

Provided herein are methods of manufacturing immune effector cells (for example, T cells or NK cells) engineered to express a CAR, for example, a CAR described herein, compositions comprising such cells, and methods of using such cells for treating a disease, such as cancer, in a subject. In some embodiments, the methods disclosed herein may manufacture immune effector cells engineered to express a CAR in less than 24 hours. Without wishing to be bound by theory, the methods provided herein preserve the undifferentiated phenotype of T cells, such as naïve T cells, during the manufacturing process. These CAR-expressing cells with an undifferentiated phenotype may persist longer and/or expand better in vivo after infusion. In some embodiments, CART cells produced by the manufacturing methods provided herein comprise a higher percentage of stem cell memory T cells, compared to CART cells produced by the traditional manufacturing process, e.g., as measured using scRNA-seq (e.g., as measured using methods described in Example 10 with respect to FIG. 39A). In some embodiments, CART cells produced by the manufacturing methods provided herein comprise a higher percentage of effector T cells, compared to CART cells produced by the traditional manufacturing process, e.g., as measured using scRNA-seq (e.g., as measured using methods described in Example 10 with respect to FIG. 39B). In some embodiments, CART cells produced by the manufacturing methods provided herein better preserve the stemness of T cells, compared to CART cells produced by the traditional manufacturing process, e.g., as measured using scRNA-seq (e.g., as measured using methods described in Example 10 with respect to FIG. 39C). In some embodiments, CART cells produced by the manufacturing methods provided herein show a lower level of hypoxia, compared to CART cells produced by the traditional manufacturing process, e.g., as measured using scRNA-seq (e.g., as measured using methods described in Example 10 with respect to FIG. 39D). In some embodiments, CART cells produced by the manufacturing methods provided herein show a lower level of autophagy, compared to CART cells produced by the traditional manufacturing process, e.g., as measured using scRNA-seq (e.g., as measured using methods described in Example 10 with respect to FIG. 39E).

In some embodiments, the methods disclosed herein do not involve using a bead, such as Dynabeads® (for example, CD3/CD28 Dynabeads®), and do not involve a de-beading step. In some embodiments, the CART cells manufactured by the methods disclosed herein may be administered to a subject with minimal ex vivo expansion, for example, less than 1 day, less than 12 hours, less than 8 hours, less than 6 hours, less than 4 hours, less than 3 hours, less than 2 hours, less than 1 hour, or no ex vivo expansion. Accordingly, the methods described herein provide a fast manufacturing process of making improved CAR-expressing cell products for use in treating a disease in a subject.

### Cytokine Process

In some embodiments, the present disclosure provides methods of making a population of cells (for example, T cells) that express a chimeric antigen receptor (CAR) comprising: (1) contacting a population of cells with a cytokine chosen from IL-2, IL-7, IL-15, IL-21, IL-6, or a combination thereof, (2) contacting the population of cells (for example, T cells) with a nucleic acid molecule (for example, a DNA or RNA molecule) encoding the CAR, thereby providing a population of cells (for example, T cells) comprising the nucleic acid molecule, and (3) harvesting the population of cells (for example, T cells) for storage (for example, reformulating the population of cells in cryopreservation media) or administration, wherein: (a) step (2) is performed together with step (1) or no later than 5 hours after the beginning of step (1), for example, no later than 1, 2, 3, 4, or 5 hours after the beginning of step (1), and step (3) is performed no later than 26 hours after the beginning of step (1), for example, no later than 22, 23, or 24 hours after the beginning of step (1), for example, no later than 24 hours after the beginning of step (1), or (b) the population of cells from step (3) are not expanded, or expanded by no more than 5, 10, 15, 20, 25, 30, 35, or 40%, for example, no more than 10%, for example, as assessed by the number of living cells, compared to the population of cells at the beginning of step (1). In some embodiments, the nucleic acid molecule in step (2) is a DNA molecule. In some embodiments, the nucleic acid molecule in step (2) is an RNA molecule. In some embodiments, the nucleic acid molecule in step (2) is on a viral vector, for example, a viral vector chosen from a lentivirus vector, an adenoviral vector, or a retrovirus vector. In some embodiments, the nucleic acid molecule in step (2) is on a non-viral vector. In some embodiments, the nucleic acid molecule in step (2) is on a plasmid. In some embodiments, the nucleic acid molecule in step (2) is not on any vector. In some embodiments, step (2) comprises transducing the population of cells (for example, T cells) with a viral vector comprising a nucleic acid molecule encoding the CAR. In some embodiments, step (2) further comprises contacting the population of cells (for example, T cells) with shRNA that targets Tet2 comprising (A) a sense strand comprising a Tet2 target sequence and (B) an antisense strand complementary to the sense strand in whole or in part or a vector encoding the shRNA. In some embodiments, sense strand comprises the Tet2 target sequence GGGTAAGCCAAGAAAGAAA (SEQ ID NO: 418). In some embodiments, the anti-sense strand comprises the reverse complement thereof, i.e. TTTCTTTCTTGGCTTACCC (SEQ ID NO: 419). In some embodiments, the vector encoding the shRNA is the same or different from the vector encoding the CAR. In some embodiments, the vector encoding the shRNA sequence comprises promoter (such as but not limited to a U6 promoter), a sense strand comprising a Tet2 target sequence, a loop, an anti-sense strand complementary to the sense strand in whole or in part, and, optionally, a polyT tail, e.g. the sequences in Table 29.

In some embodiments, the population of cells (for example, T cells) is collected from an apheresis sample (for example, a leukapheresis sample) from a subject.

In some embodiments, the apheresis sample (for example, a leukapheresis sample) is collected from the subject and shipped as a frozen sample (for example, a cryopreserved sample) to a cell manufacturing facility. The frozen apheresis sample is then thawed, and T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then seeded for CART manufacturing using the cytokine process described herein. In some embodiments, at the end of the cytokine process, the CAR T cells are cryopreserved and later thawed and administered to the subject. In some embodiments, the selected T cells (for example, CD4+ T cells and/or CD8+ T cells) undergo one or more rounds of freeze-thaw before being seeded for CART manufacturing.

In some embodiments, the apheresis sample (for example, a leukapheresis sample) is collected from the subject and shipped as a fresh product (for example, a product that is not frozen) to a cell manufacturing facility. T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then seeded for CART manufacturing using the cytokine process described herein. In some embodiments, the selected T cells (for example, CD4+ T cells and/or CD8+ T cells) undergo one or more rounds of freeze-thaw before being seeded for CART manufacturing.

In some embodiments, the apheresis sample (for example, a leukapheresis sample) is collected from the subject. T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then shipped as a frozen sample (for example, a cryopreserved sample) to a cell manufacturing facility. The selected T cells (for example, CD4+ T cells and/or CD8+ T cells) are later thawed and seeded for CART manufacturing using the cytokine process described herein.

In some embodiments, after cells (for example, T cells) are seeded, one or more cytokines (for example, one or more cytokines chosen from IL-2, IL-7, IL-15 (for example, hetIL-15 (IL15/sIL-15Ra)), IL-21, or IL-6 (for example, IL-6/sIL-6R)) as well as vectors (for example, lentiviral vectors) encoding a CAR are added to the cells. After incubation for 20-24 hours, the cells are washed and formulated for storage or administration.

Different from traditional CART manufacturing approaches, the cytokine process provided herein does not involve CD3 and/or CD28 stimulation, or ex vivo T cell expansion. T cells that are contacted with anti-CD3 and anti-CD28 antibodies and expanded extensively ex vivo tend to show differentiation towards a central memory phenotype. Without wishing to be bound by theory, the cytokine process provided herein preserves or increases the undifferentiated phenotype of T cells during CART manufacturing, generating a CART product that may persist longer after being infused into a subject.

In some embodiments, the population of cells is contacted with one or more cytokines (for example, one or more cytokines chosen from IL-2, IL-7, IL-15 (for example, hetIL-15 (IL15/sIL-15Ra)), IL-21, or IL-6 (for example, IL-6/sIL-6Ra).

In some embodiments, the population of cells is contacted with IL-2. In some embodiments, the population of cells is contacted with IL-7. In some embodiments, the population of cells is contacted with IL-15 (for example, hetIL-15 (IL15/sIL-15Ra)). In some embodiments, the population of cells is contacted with IL-21. In some embodiments, the population of cells is contacted with IL-6 (for example, IL-6/sIL-6Ra). In some embodiments, the population of cells is contacted with IL-2 and IL-7. In some embodiments, the population of cells is contacted with IL-2 and IL-15 (for example, hetIL-15 (IL15/sIL-15Ra)). In some embodiments, the population of cells is contacted with IL-2 and IL-21. In some embodiments, the population of cells is contacted with IL-2 and IL-6 (for example, IL-6/sIL-6Ra). In some embodiments, the population of cells is contacted with IL-7 and IL-15 (for example, hetIL-15 (IL15/sIL-15Ra)). In some embodiments, the population of cells is contacted with IL-7 and IL-21. In some embodiments, the population of cells is contacted with IL-7 and IL-6 (for example, IL-6/sIL-6Ra). In some embodiments, the population of cells is contacted with IL-15 (for example, hetIL-15 (IL15/sIL-15Ra)) and IL-21. In some embodiments, the population of cells is contacted with IL-15 (for example, hetIL-15 (IL15/sIL-15Ra)) and IL-6 (for example, IL-6/sIL-6Ra). In some embodiments, the population of cells is contacted with IL-21 and IL-6 (for example, IL-6/sIL-6Ra). In some embodiments, the population of cells is contacted with IL-7, IL-15 (for example, hetIL-15 (IL15/sIL-15Ra)), and IL-21. In some embodiments, the population of cells is further contacted with a LSD1 inhibitor. In some embodiments, the population of cells is further contacted with a MALT1 inhibitor.

In some embodiments, the population of cells is contacted with 20, 30, 40, 50, 60, 70, 80, 90, 100, 110, 120, 130, 140, 150, 160, 170, 180, 190, 200, 210, 220, 230, 240, 250, 260, 270, 280, 290, or 300 U/ml of IL-2. In some embodiments, the population of cells is contacted with 1, 2, 3, 4, 5, 6, 7, 8, 9, 10, 11, 12, 13, 14, 15, 16, 17, 18, 19, or 20 ng/ml of IL-7. In some embodiments, the population of cells is contacted with 1, 2, 3, 4, 5, 6, 7, 8, 9, 10, 11, 12, 13, 14, 15, 16, 17, 18, 19, or 20 ng/ml of IL-15.

In some embodiments, the population of cells is contacted with a nucleic acid molecule encoding a CAR. In some embodiments, the population of cells is transduced with a DNA molecule encoding a CAR.

In some embodiments, contacting the population of cells with the nucleic acid molecule encoding the CAR occurs simultaneously with contacting the population of cells with the one or more cytokines described above. In some embodiments, contacting the population of cells with the nucleic acid molecule encoding the CAR occurs no later than 1, 1.5, 2, 2.5, 3, 3.5, 4, 4.5, 5, 5.5, 6, 6.5, 7, 7.5, 8, 8.5, 9, 9.5 or 10 hours after the beginning of contacting the population of cells with the one or more cytokines described above. In some embodiments, contacting the population of cells with the nucleic acid molecule encoding the CAR occurs no later than 5 hours after the beginning of contacting the population of cells with the one or more cytokines described above. In some embodiments, contacting the population of cells with the nucleic acid molecule encoding the CAR occurs no later than 4 hours after the beginning of contacting the population of cells with the one or more cytokines described above. In some embodiments, contacting the population of cells with the nucleic acid molecule encoding the CAR occurs no later than 3 hours after the beginning of contacting the population of cells with the one or more cytokines described above. In some embodiments, contacting the population of cells with the nucleic acid molecule encoding the CAR occurs no later than 2 hours after the beginning of contacting the population of cells with the one or more cytokines described above. In some embodiments, contacting the population of cells with the nucleic acid molecule encoding the CAR occurs no later than 1 hour after the beginning of contacting the population of cells with the one or more cytokines described above.

In some embodiments, the population of cells is harvested for storage or administration.

In some embodiments, the population of cells is harvested for storage or administration no later than 72, 60, 48, 36, 32, 31, 30, 29, 28, 27, 26, 25, 24, 23, 22, 21, 20, 19, or 18 hours after the beginning of contacting the population of cells with the one or more cytokines described above. In some embodiments, the population of cells is harvested for storage or administration no later than 26 hours after the beginning of contacting the population of cells with the one or more cytokines described above. In some embodiments, the population of cells is harvested for storage or administration no later than 25 hours after the beginning of contacting the population of cells with the one or more cytokines described above. In some embodiments, the population of cells is harvested for storage or administration no later than 24 hours after the beginning of contacting the population of cells with the one or more cytokines described above. In some embodiments, the population of cells is harvested for storage or administration no later than 23 hours after the beginning of contacting the population of cells with the one or more cytokines described above. In some embodiments, the population of cells is harvested for storage or administration no later than 22 hours after the beginning of contacting the population of cells with the one or more cytokines described above.

In some embodiments, the population of cells is not expanded ex vivo.

In some embodiments, the population of cells is expanded by no more than 5, 6, 7, 8, 9, 10, 11, 12, 13, 14, 15, 16, 17, 18, 19, 20, 25, 30, 35, 40, 45, 50, 55, or 60%, for example, as assessed by the number of living cells, compared to the population of cells before it is contacted with the one or more cytokines described above. In some embodiments, the population of cells is expanded by no more than 5%, for example, as assessed by the number of living cells, compared to the population of cells before it is contacted with the one or more cytokines described above. In some embodiments, the population of cells is expanded by no more than 10%, for example, as assessed by the number of living cells, compared to the population of cells before it is contacted with the one or more cytokines described above. In some embodiments, the population of cells is expanded by no more than 15%, for example, as assessed by the number of living cells, compared to the population of cells before it is contacted with the one or more cytokines described above. In some embodiments, the population of cells is expanded by no more than 20%, for example, as assessed by the number of living cells, compared to the population of cells before it is contacted with the one or more cytokines described above. In some embodiments, the population of cells is expanded by no more than 25%, for example, as assessed by the number of living cells, compared to the population of cells before it is contacted with the one or more cytokines described above. In some embodiments, the population of cells is expanded by no more than 30%, for example, as assessed by the number of living cells, compared to the population of cells before it is contacted with the one or more cytokines described above. In some embodiments, the population of cells is expanded by no more than 35%, for example, as assessed by the number of living cells, compared to the population of cells before it is contacted with the one or more cytokines described above. In some embodiments, the population of cells is expanded by no more than 40%, for example, as assessed by the number of living cells, compared to the population of cells before it is contacted with the one or more cytokines described above.

In some embodiments, the population of cells is expanded by no more than 1, 1.5, 2, 2.5, 3, 3.5, 4, 4.5, 5, 6, 7, 8, 9, 10, 11, 12, 16, 20, 24, 36, or 48 hours, for example, as assessed by the number of living cells, compared to the population of cells before it is contacted with the one or more cytokines described above.

In some embodiments, the population of cells is not contacted in vitro with an agent that stimulates a CD3/TCR complex (for example, an anti-CD3 antibody) and/or an agent that stimulates a costimulatory molecule and/or growth factor receptor on the surface of the cells (for example, an anti-CD28 antibody), or if contacted, the contacting step is less than 1, 1.5, 2, 2.5, 3, 3.5, 4, 4.5, or 5 hours.

In some embodiments, the population of cells is contacted in vitro with an agent that stimulates a CD3/TCR complex (for example, an anti-CD3 antibody) and/or an agent that stimulates a costimulatory molecule and/or growth factor receptor on the surface of the cells (for example, an anti-CD28 antibody) for 20, 21, 22, 23, 24, 25, 26, 27, or 28 hours.

In some embodiments, the population of cells manufactured using the cytokine process provided herein shows a higher percentage of naïve cells among CAR-expressing cells (for example, at least 5, 6, 7, 8, 9, 10, 11, 12, 13, 14, 15, 16, 17, 18, 19, 20, 25, 30, 35, 40, 45, 50, 55, or 60% higher), compared with cells made by an otherwise similar method which further comprises contacting the population of cells with, for example, an agent that binds a CD3/TCR complex (for example, an anti-CD3 antibody) and/or an agent that binds a costimulatory molecule on the surface of the cells (for example, an anti-CD28 antibody).

In some embodiments, the cytokine process provided herein is conducted in cell media comprising no more than 0, 0.5, 1, 1.5, 2, 2.5, 3, 3.5, 4, 4.5, 5, 5.5, 6, 6.5, 7, 7.5, or 8% serum. In some embodiments, the cytokine process provided herein is conducted in cell media comprising a LSD1 inhibitor, a MALT1 inhibitor, or a combination thereof.

### Activation Process

In some embodiments, the present disclosure provides methods of making a population of cells (for example, T cells) that express a chimeric antigen receptor (CAR) comprising: (i) contacting a population of cells (for example, T cells, for example, T cells isolated from a frozen or fresh leukapheresis product) with (A) an agent that stimulates a CD3/TCR complex and/or (B) an agent that stimulates a costimulatory molecule and/or growth factor receptor on the surface of the cells; (ii) contacting the population of cells (for example, T cells) with a nucleic acid molecule (for example, a DNA or RNA molecule) encoding the CAR, thereby providing a population of cells (for example, T cells) comprising the nucleic acid molecule, and (iii) harvesting the population of cells (for example, T cells) for storage (for example, reformulating the population of cells in cryopreservation media) or administration, wherein: (a) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 26 hours after the beginning of step (i), for example, no later than 22, 23, or 24 hours after the beginning of step (i), for example, no later than 24 hours after the beginning of step (i); (b) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 30, 36, or 48 hours after the beginning of step (ii), for example, no later than 22, 23, 24, 25, 26, 27, 28, 29, 30, 31, 32, 33, 34, 35, 36, 37, 38, 39, 40, 41, 42, 43, 44, 45, 46, 47, or 48 hours after the beginning of step (ii); or (c) the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i). In some embodiments, the nucleic acid molecule in step (ii) is a DNA molecule. In some embodiments, the nucleic acid molecule in step (ii) is an RNA molecule. In some embodiments, the nucleic acid molecule in step (ii) is on a viral vector, for example, a viral vector chosen from a lentivirus vector, an adenoviral vector, or a retrovirus vector. In some embodiments, the nucleic acid molecule in step (ii) is on a non-viral vector. In some embodiments, the nucleic acid molecule in step (ii) is on a plasmid. In some embodiments, the nucleic acid molecule in step (ii) is not on any vector. In some embodiments, step (ii) comprises transducing the population of cells (for example, T cells) a viral vector comprising a nucleic acid molecule encoding the CAR. In some embodiments, step (2) further comprises contacting the population of cells (for example, T cells) with shRNA that targets Tet2 comprising (A) a sense strand comprising a Tet2 target sequence and (B) an antisense strand complementary to the sense strand in whole or in part or a vector encoding the shRNA. In some embodiments, sense strand comprises the Tet2 target sequence GGGTAAGCCAAGAAAGAAA. In some embodiments, the anti-sense strand comprises the reverse complement thereof, i.e. TTTCTTTCTTGGCTTACCC. In some embodiments, the vector encoding the shRNA is the same or different from the vector encoding the CAR. In some embodiments, the vector encoding the shRNA sequence comprises promoter (such as but not limited to a U6 promoter), a sense strand comprising a Tet2 target sequence, a loop, an anti-sense strand complementary to the sense strand in whole or in part, and, optionally, a polyT tail, e.g. the sequences in Table 29.

In some embodiments, the population of cells (for example, T cells) is collected from an apheresis sample (for example, a leukapheresis sample) from a subject.

In some embodiments, the apheresis sample (for example, a leukapheresis sample) is collected from the subject and shipped as a frozen sample (for example, a cryopreserved sample) to a cell manufacturing facility. Then the frozen apheresis sample is thawed, and T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then seeded for CART 30) manufacturing using the activation process described herein. In some embodiments, the selected T cells (for example, CD4+ T cells and/or CD8+ T cells) undergo one or more rounds of freeze-thaw before being seeded for CART manufacturing.

In some embodiments, the apheresis sample (for example, a leukapheresis sample) is collected from the subject and shipped as a fresh product (for example, a product that is not frozen) to a cell manufacturing facility. T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then seeded for CART manufacturing using the activation process described herein. In some embodiments, the selected T cells (for example, CD4+ T cells and/or CD8+ T cells) undergo one or more rounds of freeze-thaw before being seeded for CART manufacturing.

In some embodiments, the apheresis sample (for example, a leukapheresis sample) is collected from the subject. T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then shipped as a frozen sample (for example, a cryopreserved sample) to a cell manufacturing facility. The selected T cells (for example, CD4+ T cells and/or CD8+ T cells) are later thawed and seeded for CART manufacturing using the activation process described herein.

In some embodiments, cells (for example, T cells) are contacted with anti-CD3 and anti-CD28 antibodies for, for example, 12 hours, followed by transduction with a vector (for example, a lentiviral vector) encoding a CAR. 24 hours after culture initiation, the cells are washed and formulated for storage or administration.

Without wishing to be bound by theory, brief CD3 and CD28 stimulation may promote efficient transduction of self-renewing T cells. Compared to traditional CART manufacturing approaches, the activation process provided herein does not involve prolonged ex vivo expansion. Similar to the cytokine process, the activation process provided herein also preserves undifferentiated T cells during CART manufacturing.

In some embodiments, the population of cells is contacted with (A) an agent that stimulates a CD3/TCR complex and/or (B) an agent that stimulates a costimulatory molecule and/or growth factor receptor on the surface of the cells.

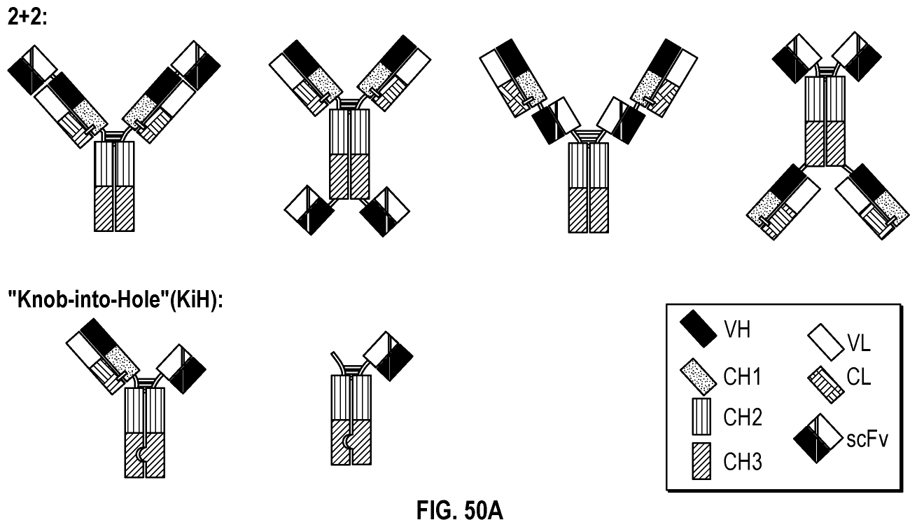

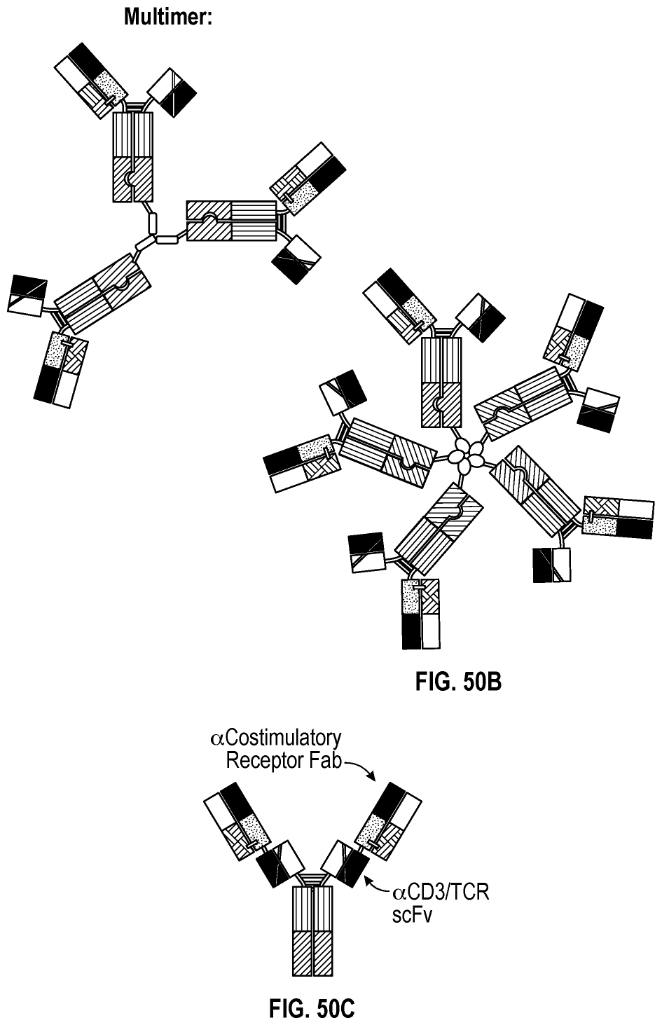

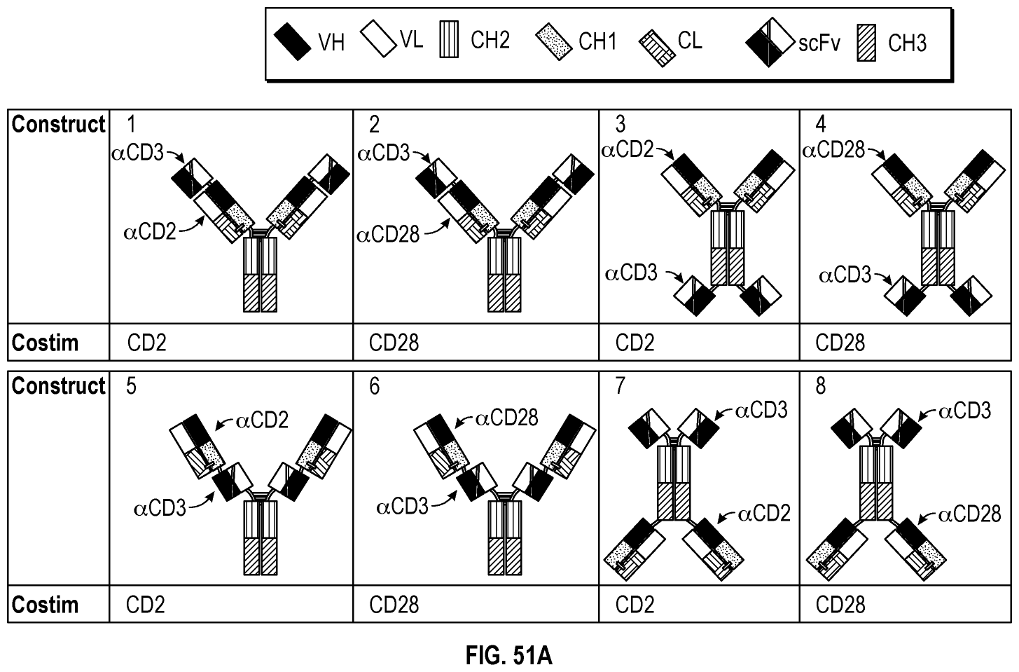

In some embodiments, the agent that stimulates a CD3/TCR complex is an agent that stimulates CD3. In some embodiments, the agent that stimulates a costimulatory molecule and/or growth factor receptor is an agent that stimulates CD28, ICOS, CD27, HVEM, LIGHT, CD40, 4-1BB, OX40, DR3, GITR, CD30, TIM1, CD2, CD226, or any combination thereof. In some embodiments, the agent that stimulates a costimulatory molecule and/or growth factor receptor is an agent that stimulates CD28. In some embodiments, the agent that stimulates a CD3/TCR complex is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand). In some embodiments, the agent that stimulates a costimulatory molecule and/or growth factor receptor is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand). In some embodiments, the agent that stimulates a CD3/TCR complex does not comprise a bead. In some embodiments, the agent that stimulates a costimulatory molecule and/or growth factor receptor does not comprise a bead. In some embodiments, the agent that stimulates a CD3/TCR complex comprises an anti-CD3 antibody. In some embodiments, the agent that stimulates a costimulatory molecule and/or growth factor receptor comprises an anti-CD28 antibody. In some embodiments, the agent that stimulates a CD3/TCR complex comprises an anti-CD3 antibody covalently attached to a colloidal polymeric nanomatrix. In some embodiments, the agent that stimulates CD3 comprises one or more of a CD3 or TCR antigen binding domain, such as but not limited to an anti-CD3 or anti-TCR antibody or an antibody fragment comprising one or more CDRs, heavy chain, and/or light chain thereof-such as but not limited to an anti-CD3 or anti-TCR antibody provided in Table 27. In some embodiments, the agent that stimulates a costimulatory molecule and/or growth factor receptor comprises an anti-CD28 antibody covalently attached to a colloidal polymeric nanomatrix. In some embodiments, the agent that stimulates a costimulatory molecule and/or growth factor receptor is an agent that stimulates CD28, ICOS, CD27, CD25, 4-1BB, IL6RA, IL6RB, or CD2. In some embodiments, the agent that stimulates a costimulatory molecule and/or growth factor receptor comprises one or more of a CD28, ICOS, CD27, CD25, 4-1BB, IL6RB, and/or CD2 antigen binding domain, such as but not limited to an anti-CD28, anti-ICOS, anti-CD27, anti-CD25, anti-4-1BB, anti-IL6RA, anti-IL6RB, or anti-CD2 antibody or an antibody fragment comprising one or more CDRs, heavy chain, and/or light chain thereof-such as but not limited to an anti-CD28, anti-ICOS, anti-CD27, anti-CD25, anti-4-1BB, anti-IL6RA, anti-IL6RB, or anti-CD2 antibody provided in Table 27. In some embodiments, the agent that stimulates a CD3/TCR complex and the agent that stimulates a costimulatory molecule and/or growth factor receptor comprise T Cell TransAct™. In some embodiments, the agent that stimulates a CD3/TCR complex and the agent that stimulates a costimulatory molecule and/or growth factor receptor are comprised in a multispecific binding molecule. In some embodiments, the multispecific binding molecule comprises a CD3 antigen binding domain and a CD28 or CD2 antigen binding domain. In some embodiments, the multispecific binding molecules comprise one or more heavy and/or light chains-such as but not limited to the heavy and/or light chains provided in Table 28. In some embodiments, the multispecific binding molecule comprises a bispecific antibody. In some embodiments, the bispecific antibody is configured in any one of the schema provided in FIG. 50A. In some embodiments, the bispecific antibody is monovalent or bivalent. In some embodiments, the bispecific antibody comprises an Fc region. In some embodiments, the Fc region of the bispecific antibody is silenced. In some embodiments, the Fc region of the bispecific antibody is silenced by a combination of amino acid substitutions selected from the group consisting of LALA, DAPA, DANAPA, LALADANAPS, LALAGA, LALASKPA, DAPASK, GADAPA, GADAPASK, LALAPG, and LALAPA. In some embodiments, the multispecific binding molecule comprises a plurality of bispecific antibodies. In some embodiments, one or more of the plurality of bispecific antibodies is monovalent. In some embodiments, one or more of the plurality of bispecific antibodies comprises an Fc region. In some embodiments, the Fc region of the one or more of the plurality of bispecific antibodies is silenced. In some embodiments, one or more of the plurality of bispecific antibodies are conjugated together into a multimer. In some embodiments, the multimer is configured in any one of the schema provided in FIG. 50B.

In some embodiments, the matrix comprises or consists of a polymeric, for example, biodegradable or biocompatible inert material, for example, which is non-toxic to cells. In some embodiments, the matrix is composed of hydrophilic polymer chains, which obtain maximal mobility in aqueous solution due to hydration of the chains. In some embodiments, the mobile matrix may be of collagen, purified proteins, purified peptides, polysaccharides, glycosaminoglycans, or extracellular matrix compositions. A polysaccharide may include for example, cellulose ethers, starch, gum arabic, agarose, dextran, chitosan, hyaluronic acid, pectins, xanthan, guar gum or alginate. Other polymers may include polyesters, polyethers, polyacrylates, polyacrylamides, polyamines, polyethylene imines, polyquaternium polymers, polyphosphazenes, polyvinylalcohols, polyvinylacetates, polyvinylpyrrolidones, block copolymers, or polyurethanes. In some embodiments, the mobile matrix is a polymer of dextran.

In some embodiments, the population of cells is contacted with a nucleic acid molecule encoding a CAR. In some embodiments, the population of cells is transduced with a DNA molecule encoding a CAR.

In some embodiments, contacting the population of cells with the nucleic acid molecule encoding the CAR occurs simultaneously with contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0.5 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 20 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 19 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 18 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 17 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 16 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 15 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 14 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 14 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 13 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 12 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 11 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 10 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 9 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 8 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 7 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 6 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 5 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 4 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 3 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 2 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 1 hour after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 30 minutes after the beginning of contacting the population of cells with the agent that stimulates a CD3/TCR complex and/or the agent that stimulates a costimulatory molecule and/or growth factor receptor on the surface of the cells described above.

In some embodiments, the population of cells is harvested for storage or administration.

In some embodiments, the population of cells is harvested for storage or administration no later than 72, 60, 48, 36, 32, 31, 30, 29, 28, 27, 26, 25, 24, 23, 22, 21, 20, 19, or 18 hours after the beginning of contacting the population of cells with the agent that stimulates a CD3/TCR complex and/or the agent that stimulates a costimulatory molecule and/or growth factor receptor on the surface of the cells described above. In some embodiments, the population of cells is harvested for storage or administration no later than 26 hours after the beginning of contacting the population of cells with the agent that stimulates a CD3/TCR complex and/or the agent that stimulates a costimulatory molecule and/or growth factor receptor on the surface of the cells described above. In some embodiments, the population of cells is harvested for storage or administration no later than 25 hours after the beginning of contacting the population of cells with the agent that stimulates a CD3/TCR complex and/or the agent that stimulates a costimulatory molecule and/or growth factor receptor on the surface of the cells described above. In some embodiments, the population of cells is harvested for storage or administration no later than 24 hours after the beginning of contacting the population of cells with the agent that stimulates a CD3/TCR complex and/or the agent that stimulates a costimulatory molecule and/or growth factor receptor on the surface of the cells described above. In some embodiments, the population of cells is harvested for storage or administration no later than 23 hours after the beginning of contacting the population of cells with the agent that stimulates a CD3/TCR complex and/or the agent that stimulates a costimulatory molecule and/or growth factor receptor on the surface of the cells described above. In some embodiments, the population of cells is harvested for storage or administration no later than 22 hours after the beginning of contacting the population of cells with the agent that stimulates a CD3/TCR complex and/or the agent that stimulates a costimulatory molecule and/or growth factor receptor on the surface of the cells described above.

In some embodiments, the population of cells is not expanded ex vivo.

In some embodiments, the population of cells is expanded by no more than 5, 6, 7, 8, 9, 10, 11, 12, 13, 14, 15, 16, 17, 18, 19, 20, 25, 30, 35, 40, 45, 50, 55, or 60%, for example, as assessed by the number of living cells, compared to the population of cells before it is contacted with the agent that stimulates a CD3/TCR complex and/or the agent that stimulates a costimulatory molecule and/or growth factor receptor on the surface of the cells described above. In some embodiments, the population of cells is expanded by no more than 5%, for example, as assessed by the number of living cells, compared to the population of cells before it is contacted with the agent that stimulates a CD3/TCR complex and/or the agent that stimulates a costimulatory molecule and/or growth factor receptor on the surface of the cells described above. In some embodiments, the population of cells is expanded by no more than 10%, for example, as assessed by the number of living cells, compared to the population of cells before it is contacted with the agent that stimulates a CD3/TCR complex and/or the agent that stimulates a costimulatory molecule and/or growth factor receptor on the surface of the cells described above. In some embodiments, the population of cells is expanded by no more than 15%, for example, as assessed by the number of living cells, compared to the population of cells before it is contacted with the agent that stimulates a CD3/TCR complex and/or the agent that stimulates a costimulatory molecule and/or growth factor receptor on the surface of the cells described above. In some embodiments, the population of cells is expanded by no more than 20%, for example, as assessed by the number of living cells, compared to the population of cells before it is contacted with the agent that stimulates a CD3/TCR complex and/or the agent that stimulates a costimulatory molecule and/or growth factor receptor on the surface of the cells described above. In some embodiments, the population of cells is expanded by no more than 25%, for example, as assessed by the number of living cells, compared to the population of cells before it is contacted with the agent that stimulates a CD3/TCR complex and/or the agent that stimulates a costimulatory molecule and/or growth factor receptor on the surface of the cells described above. In some embodiments, the population of cells is expanded by no more than 30%, for example, as assessed by the number of living cells, compared to the population of cells before it is contacted with the agent that stimulates a CD3/TCR complex and/or the agent that stimulates a costimulatory molecule and/or growth factor receptor on the surface of the cells described above. In some embodiments, the population of cells is expanded by no more than 35%, for example, as assessed by the number of living cells, compared to the population of cells before it is contacted with the agent that stimulates a CD3/TCR complex and/or the agent that stimulates a costimulatory molecule and/or growth factor receptor on the surface of the cells described above. In some embodiments, the population of cells is expanded by no more than 40%, for example, as assessed by the number of living cells, compared to the population of cells before it is contacted with the agent that stimulates a CD3/TCR complex and/or the agent that stimulates a costimulatory molecule and/or growth factor receptor on the surface of the cells described above.

In some embodiments, the population of cells is expanded by no more than 1, 1.5, 2, 2.5, 3, 3.5, 4, 4.5, 5, 6, 7, 8, 9, 10, 11, 12, 16, 20, 24, 36, or 48 hours, for example, as assessed by the number of living cells, compared to the population of cells before it is contacted with the one or more cytokines described above.

In some embodiments, the activation process is conducted in serum free cell media. In some embodiments, the activation process is conducted in cell media comprising one or more cytokines chosen from: IL-2, IL-15 (for example, hetIL-15 (IL15/sIL-15Ra)), or IL-6 (for example, IL-6/sIL-6Ra). In some embodiments, hetIL-15 comprises the amino acid sequence of
NWVNVISDLKKIEDLIQSMHIDATLYTESDVHPSCKVTAMKCFLLELQVISLESGDASIH DTVENLIILANNSLSSNGNVTESGCKECEELEEKNIKEFLQSFVHIVQMFINTSITCPPPM SVEHADIWVKSYSLYSRERYICNSGFKRKAGTSSLTECVLNKATNVAHWTTPSLKCIR DPALVHQRPAPPSTVTTAGVTPQPESLSPSGKEPAASSPSSNNTAATTAAIVPGSQLMPS KSPSTGTTEISSHESSHGTPSQTTAKNWELTASASHQPPGVYPQG (SEQ ID NO: 309). In some embodiments, hetIL-15 comprises an amino acid sequence having at least about 70, 75, 80, 85, 90, 95, or 99% identity to SEQ ID NO: 309. In some embodiments, the activation process is conducted in cell media comprising a LSD1 inhibitor. In some embodiments, the activation process is conducted in cell media comprising a MALT1 inhibitor. In some embodiments, the serum free cell media comprises a serum replacement. In some embodiments, the serum replacement is CTS™ Immune Cell Serum Replacement (ICSR). In some embodiments, the level of ICSR can be, for example, up to 5%, for example, about 1%, 2%, 3%, 4%, or 5%. Without wishing to be bound by theory, using cell media, for example, Rapid Media shown in Table 21 or Table 25, comprising ICSR, for example, 2% ICSR, may improve cell viability during a manufacture process described herein.

In some embodiments, the present disclosure provides methods of making a population of cells (for example, T cells) that express a chimeric antigen receptor (CAR) comprising: (a) providing an apheresis sample (for example, a fresh or cryopreserved leukapheresis sample) collected from a subject; (b) selecting T cells from the apheresis sample (for example, using negative selection, positive selection, or selection without beads); (c) seeding isolated T cells at, for example, 1×10⁶ to 1×10⁷ cells/mL; (d) contacting T cells with an agent that stimulates T cells, for example, an agent that stimulates a CD3/TCR complex and/or an agent that stimulates a costimulatory molecule and/or growth factor receptor on the surface of the cells (for example, contacting T cells with anti-CD3 and/or anti-CD28 antibody, for example, contacting T cells with TransAct); (e) contacting T cells with a nucleic acid molecule (for example, a DNA or RNA molecule) encoding the CAR (for example, contacting T cells with a virus comprising a nucleic acid molecule encoding the CAR) for, for example, 6-48 hours, for example, 20-28 hours; and (f) washing and harvesting T cells for storage (for example, reformulating T cells in cryopreservation media) or administration. In some embodiments, step (f) is performed no later than 30, 36, or 48 hours after the beginning of step (d) or (e), for example, no later than 22, 23, 24, 25, 26, 27, 28, 29, 30, 31, 32, 33, 34, 35, 36, 37, 38, 39, 40, 41, 42, 43, 44, 45, 46, 47, or 48 hours after the beginning of step (d) or (e).

In some embodiments of the aforementioned methods, the methods are performed in a closed system. In some embodiments, T cell separation, activation, transduction, incubation, and washing are all performed in a closed system. In some embodiments of the aforementioned methods, the methods are performed in separate devices. In some embodiments, T cell separation, activation and transduction, incubation, and washing are performed in separate devices.

In some embodiments of the aforementioned methods, the methods further comprise adding an adjuvant or a transduction enhancement reagent in the cell culture medium to enhance transduction efficiency. In some embodiments, the adjuvant or transduction enhancement reagent comprises a cationic polymer. In some embodiments, the adjuvant or transduction enhancement reagent is chosen from: LentiBOOST™ (Sirion Biotech), vectofusin-1, F108 (Poloxamer 338 or Pluronic® F-38), protamine sulfate, hexadimethrine bromide (Polybrene), PEA, Pluronic F68, Pluronic F127, Synperonic or LentiTrans™. In some embodiments, the transduction enhancement reagent is LentiBOOST™ (Sirion Biotech). In some embodiments, the transduction enhancement reagent is F108 (Poloxamer 338 or Pluronic® F-38)

In some embodiments of the aforementioned methods, the transducing the population of cells (for example, T cells) with a viral vector comprises subjecting the population of cells and viral vector to a centrifugal force under conditions such that transduction efficiency is enhanced. In an embodiment, the cells are transduced by spinoculation.

In some embodiments of the aforementioned methods, cells (e.g., T cells) are activated and transduced in a cell culture flask comprising a gas-permeable membrane at the base that supports large media volumes without substantially compromising gas exchange. In some embodiments, cell growth is achieved by providing access, e.g., substantially uninterrupted access, to nutrients through convection.

### Anti-CD28 Antibody Molecules

In some embodiments, the anti-CD28 antibody, e.g., an anti-CD28 antibody to be used in a multispecific binding molecule described herein, comprises at least one antigen-binding region, e.g., a variable region or an antigen-binding fragment thereof, from anti-CD28 (2) as described in Table 27. In some embodiments, the anti-CD28 antibody molecule comprises one or two variable regions from anti-CD28 (2), as described in Table 27. In some embodiments, the anti-CD28 antibody comprises a heavy chain variable region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3, wherein the HCDR1, HCDR2, HCDR3, LCDR1, LCDR2, and LCDR3 comprise the amino acid sequences of SEQ ID NOs: 538, 539, 540, 530, 531, and 532, respectively; the HCDR1, HCDR2, HCDR3, LCDR1, LCDR2, and LCDR3 comprise the amino acid sequences of SEQ ID NOs: 541, 539, 540, 530, 531, and 532, respectively; the HCDR1, HCDR2, HCDR3, LCDR1, LCDR2, and LCDR3 comprise the amino acid sequences of SEQ ID NOs: 542, 543, 540, 533, 534, and 535, respectively; or the HCDR1, HCDR2, HCDR3, LCDR1, LCDR2, and LCDR3 comprise the amino acid sequences of SEQ ID NOs: 544, 545, 546, 536, 534, and 532, respectively.

In some embodiments, the anti-CD28 antibody molecule comprises a VH comprising the amino acid sequence of SEQ ID NO: 547 or 548, or a sequence having at least about 80%, 85%, 90%, 92%, 95%, 97%, 98%, or 99% sequence identity to SEQ ID NO: 547 or 548. In some embodiments, the anti-CD28 antibody comprises a VL comprising the amino acid sequence of SEQ ID NO: 537, or a sequence having at least about 80%, 85%, 90%, 92%, 95%, 97%, 98%, or 99% sequence identity thereto. In some embodiments, the anti-CD28 antibody comprises a VH and a VL comprising the amino acid sequences of SEQ ID NOs: 547 and 537, respectively, or a sequence having at least about 80%, 85%, 90%, 92%, 95%, 97%, 98%, or 99% sequence identity to any of the aforesaid sequences. In some embodiments, the anti-CD28 antibody comprises a VH and a VL comprising the amino acid sequences of SEQ ID NOs: 548 and 537, respectively, or a sequence having at least about 80%, 85%, 90%, 92%, 95%, 97%, 98%, or 99% sequence identity to any of the aforesaid sequences.

It is understood that an anti-CD28 antibody described herein can be used in the context of a multispecific binding molecule, e.g., with an additional binding domain, e.g., an anti-CD3 binding domain described herein. It is also understood that anti-CD28 antibody described herein can be used in other contexts, e.g., as a monospecific antibody.

### Multispecific Binding Molecule Reagents

In some embodiments, the agent that stimulates a CD3/TCR complex is an agent that stimulates CD3. In some embodiments, the agent that stimulates CD3 comprises one or more of a CD3 or TCR antigen binding domain, such as but not limited to an anti-CD3 or anti-TCR antibody or an antibody fragment comprising one or more CDRs, VH, heavy chain, VL, and/or light chain thereof.

Anti-CD3 antibody sequences and methods of making such antibodies are known in the art. Non-limiting examples of anti-CD3 antibody sequences, along with the relevant CDR, VH, and VL sequences are provided in Table 27. In some embodiments, the anti-CD3 binding domain comprises a VH and a VL comprising the amino acid sequences of SEQ ID NOs: 437 and 427, respectively. In some embodiments, the anti-CD3 binding domain comprises a VH and a VL comprising the amino acid sequences of SEQ ID NOs: 456 and 445, respectively. In some embodiments, the anti-CD3 binding domain comprises a VH and a VL comprising the amino acid sequences of SEQ ID NOs: 457 and 446, respectively. In some embodiments, the anti-CD3 binding domain comprises a VH and a VL comprising the amino acid sequences of SEQ ID NOs: 475 and 467, respectively. In some embodiments, the anti-CD3 binding domain comprises a VH and a VL comprising the amino acid sequences of SEQ ID NOs: 476 and 468, respectively. In some embodiments, the anti-CD3 binding domain comprises a VH and a VL comprising the amino acid sequences of SEQ ID NOs: 494 and 484, respectively.

Anti-TCR antibody sequences and methods of making such antibodies are known in the art. Non-limiting examples of anti-TCR antibody sequences, along with the relevant CDR, VH, and VL sequences are provided in Table 27.

In some embodiments, the agent that stimulates a costimulatory molecule and/or growth factor receptor is an agent that stimulates CD28, ICOS, CD27, CD25, 4-1BB, IL6RA, IL6RB, or CD2. In some embodiments, the agent that stimulates a costimulatory molecule and/or growth factor receptor comprises one or more of a CD28, ICOS, CD27, CD25, 4-1BB, IL6RB, and/or CD2 antigen binding domain, such as but not limited to an anti-CD28, anti-ICOS, anti-CD27, anti-CD25, anti-4-1BB, anti-IL6RA, anti-IL6RB, or anti-CD2 antibody or an antibody fragment comprising one or more CDRs, heavy chain, and/or light chain thereof.

Anti-CD28 antibody sequences and methods of making such antibodies are known in the art. Non-limiting examples of anti-CD28 antibody sequences, along with the relevant CDR, VH, VL, HC and LC sequences are provided in Table 27. Anti-ICOS antibody sequences and methods of making such antibodies are known in the art. Non-limiting examples of anti-ICOS antibody sequences, along with the relevant CDR, VH, VL, and LC sequences are provided in Table 27.

Anti-CD27 antibody sequences and methods of making such antibodies are known in the art. Non-limiting examples of anti-CD27 antibody sequences, along with the relevant CDR, VH, and VL sequences are provided in Table 27.

Anti-CD25 antibody sequences and methods of making such antibodies are known in the art. Non-limiting examples of anti-CD25 antibody sequences, along with the relevant CDR, VH, VL, HC, and LC sequences are provided in Table 27.

Anti-4-1BB antibody sequences and methods of making such antibodies are known in the art. Non-limiting examples of anti-4-IBB antibody sequences, along with the relevant CDR, VH, and VL sequences are provided in Table 27.

Anti-IL6RA antibody sequences and methods of making such antibodies are known in the art. Non-limiting examples of IL6RA antibody sequences, along with the relevant CDR, VH, and VL sequences are provided in Table 27.

Anti-IL6RB antibody sequences and methods of making such antibodies are known in the art. Non-limiting examples of IL6RB antibody sequences, along with the relevant CDR, VH, and VL sequences are provided in Table 27.

Anti-CD2 antibody sequences and methods of making such antibodies are known in the art. Non-limiting examples of anti-CD2 antibody sequences, along with the relevant CDR, VH, VL, HC and LC sequences are provided in Table 27.

TABLE 27
Exemplary antibody, CDR, heavy chain variable region (VH), light chain
variable region (VL), heavy chain (HC), and light chain (LC) sequences
by target antigen

| SEQ ID NO | Description | Amino acid sequence |
|---|---|---|
| ANTI-CD3 (1) | | |
| SEQ ID NO: 420 | LCDR1 | SASSSVSYMN |
| (Combined) | | |
| SEQ ID NO: 421 | LCDR2 | DTSKLAS |
| (Combined) | | |
| SEQ ID NO: 422 | LCDR3 | QQWSSNPFT |
| (Combined) | | |
| SEQ ID NO: 420 | LCDR1 | SASSSVSYMN |
| (Kabat) | | |
| SEQ ID NO: 421 | LCDR2 | DTSKLAS |
| (Kabat) | | |
| SEQ ID NO: 422 | LCDR3 | QQWSSNPFT |
| (Kabat) | | |
| SEQ ID NO: 423 | LCDR1 | SSSVSY |
| (Chothia) | | |
| SEQ ID NO: 424 | LCDR2 | DTS |
| (Chothia) | | |
| SEQ ID NO: 425 | LCDR3 | WSSNPF |
| (Chothia) | | |
| SEQ ID NO: 426 | LCDR1 | SSVSY |
| (IMGT) | | |
| SEQ ID NO: 424 | LCDR2 | DTS |
| (IMGT) | | |
| SEQ ID NO: 422 | LCDR3 | QQWSSNPFT |
| (IMGT) | | |
| SEQ ID NO: 427 | VL | DIQMTQSPSSLSASVGDRVTITCSASSSVSYMNWYQ |
| | | QTPGKAPKRWIYDTSKLASGVPSRFSGSGSGTDYTFT |
| | | ISSLQPEDIATYYCQQWSSNPFTFGQGTKLQIT |
| SEQ ID NO: 428 | HCDR1 | GYTFTRYTMH |
| (Combined) | | |
| SEQ ID NO: 429 | HCDR2 | YINPSRGYTNYNQKVKD |
| (Combined) | | |
| SEQ ID NO: 430 | HCDR3 | YYDDHYSLDY |
| (Combined) | | |
| SEQ ID NO: 431 | HCDR1 | RYTMH |
| (Kabat) | | |
| SEQ ID NO: 429 | HCDR2 | YINPSRGYTNYNQKVKD |
| (Kabat) | | |
| SEQ ID NO: 430 | HCDR3 | YYDDHYSLDY |
| (Kabat) | | |
| SEQ ID NO: 432 | HCDR1 | GYTFTRY |
| (Chothia) | | |
| SEQ ID NO: 433 | HCDR2 | NPSRGY |
| (Chothia) | | |
| SEQ ID NO: 430 | HCDR3 | YYDDHYSLDY |
| (Chothia) | | |
| SEQ ID NO: 434 | HCDR1 | GYTFTRYT |
| (IMGT) | | |
| SEQ ID NO: 435 | HCDR2 | INPSRGYT |
| (IMGT) | | |
| SEQ ID NO: 436 | HCDR3 | ARYYDDHYSLDY |
| (IMGT) | | |
| SEQ ID NO: 437 | VH | QVQLVQSGGGVVQPGRSLRLSCKASGYTFTRYTMH |
| | | WVRQAPGKGLEWIGYINPSRGYTNYNQKVKDRFTIS |
| | | RDNSKNTAFLQMDSLRPEDTGVYFCARYYDDHYSL |
| | | DYWGQGTPVTVSS |
| ANTI-CD3 (2) | | |
| SEQ ID NO: 438 | LCDR1 | GSSTGAVTSGNYPN |
| (Combined) | | |
| SEQ ID NO: 439 | LCDR2 | GTKFLAP |
| (Combined) | | |
| SEQ ID NO: 440 | LCDR3 | VLWYSNRWV |
| (Combined) | | |
| SEQ ID NO: 438 | LCDR1 | GSSTGAVTSGNYPN |
| (Kabat) | | |
| SEQ ID NO: 439 | LCDR2 | GTKFLAP |
| (Kabat) | | |
| SEQ ID NO: 440 | LCDR3 | VLWYSNRWV |
| (Kabat) | | |
| SEQ ID NO: 441 | LCDR1 | STGAVTSGNY |
| (Chothia) | | |
| SEQ ID NO: 442 | LCDR2 | GTK |
| (Chothia) | | |
| SEQ ID NO: 443 | LCDR3 | WYSNRW |
| (Chothia) | | |
| SEQ ID NO: 444 | LCDR1 | TGAVTSGNY |
| (IMGT) | | |
| SEQ ID NO: 442 | LCDR2 | GTK |
| (IMGT) | | |
| SEQ ID NO: 440 | LCDR3 | VLWYSNRWV |
| (IMGT) | | |
| SEQ ID NO: 445 | VL | QTVVTQEPSLTVSPGGTVTLTCGSSTGAVTSGNYPN |
| | | WVQQKPGQAPRGLIGGTKFLAPGTPARFSGSLLGGK |
| | | AALTLSGVQPEDEAEYYCVLWYSNRWVFGGGTKLT |
| | | VL |
| SEQ ID NO: 446 | VL | QTVVTQEPSLTVSPGGTVTLTCGSSTGAVTSGNYPN |
| | | WVQQKPGQAPRGLIGGTKFLAPGTPARFSGSLLGGK |
| | | AALTLSGVQPEDEAEYYCVLWYSNRWVFGCGTKLT |
| | | VL |
| SEQ ID NO: 447 | HCDR1 | GFTFNKYAMN |
| (Combined) | | |
| SEQ ID NO: 448 | HCDR2 | RIRSKYNNYATYYADSVKD |
| (Combined) | | |
| SEQ ID NO: 449 | HCDR3 | HGNFGNSYISYWAY |
| (Combined) | | |
| SEQ ID NO: 450 | HCDR1 | KYAMN |
| (Kabat) | | |
| SEQ ID NO: 448 | HCDR2 | RIRSKYNNYATYYADSVKD |
| (Kabat) | | |
| SEQ ID NO: 449 | HCDR3 | HGNFGNSYISYWAY |
| (Kabat) | | |
| SEQ ID NO: 451 | HCDR1 | GFTFNKY |
| (Chothia) | | |
| SEQ ID NO: 452 | HCDR2 | RSKYNNYA |
| (Chothia) | | |
| SEQ ID NO: 449 | HCDR3 | HGNFGNSYISYWAY |
| (Chothia) | | |
| SEQ ID NO: 453 | HCDR1 | GFTFNKYA |
| (IMGT) | | |
| SEQ ID NO: 454 | HCDR2 | IRSKYNNYAT |
| (IMGT) | | |
| SEQ ID NO: 455 | HCDR3 | VRHGNFGNSYISYWAY |
| (IMGT) | | |
| SEQ ID NO: 456 | VH | EVQLVESGGGLVQPGGSLKLSCAASGFTFNKYAMN |
| | | WVRQAPGKGLEWVARIRSKYNNYATYYADSVKDR |
| | | FTISRDDSKNTAYLQMNNLKTEDTAVYYCVRHGNF |
| | | GNSYISYWAYWGQGTLVTVSS |
| SEQ ID NO: 457 | VH | EVQLVESGGGLVQPGGSLKLSCAASGFTFNKYAMN |
| | | WVRQAPGKCLEWVARIRSKYNNYATYYADSVKDR |
| | | FTISRDDSKNTAYLQMNNLKTEDTAVYYCVRHGNF |
| | | GNSYISYWAYWGQGTLVTVSS |
| ANTI-CD3 (3) | | |
| SEQ ID NO: 458 | LCDR1 | RSSTGAVTTSNYAN |
| (Combined) | | |
| SEQ ID NO: 459 | LCDR2 | GTNKRAP |
| (Combined) | | |
| SEQ ID NO: 460 | LCDR3 | ALWYSNLWV |
| (Combined) | | |
| SEQ ID NO: 458 | LCDR1 | RSSTGAVTTSNYAN |
| (Kabat) | | |
| SEQ ID NO: 459 | LCDR2 | GTNKRAP |
| (Kabat) | | |
| SEQ ID NO: 460 | LCDR3 | ALWYSNLWV |
| (Kabat) | | |
| SEQ ID NO: 461 | LCDR1 | STGAVTTSNY |
| (Chothia) | | |
| SEQ ID NO: 462 | LCDR2 | GTN |
| (Chothia) | | |
| SEQ ID NO: 463 | LCDR3 | WYSNLW |
| (Chothia) | | |
| SEQ ID NO: 464 | LCDR1 | TGAVTTSNY |
| (IMGT) | | |
| SEQ ID NO: 465 | LCDR2 | GGT |
| (IMGT) | | |
| SEQ ID NO: 466 | LCDR3 | ALWYSNL |
| (IMGT) | | |
| SEQ ID NO: 467 | VL | QAVVTQEPSLTVSPGGTVTLTCRSSTGAVTTSNYAN |
| | | WVQQKPGQAPRGLIGGTNKRAPWTPARFSGSLLGD |
| | | KAALTLSGAQPEDEAEYFCALWYSNLWVFGGGTKL |
| | | TVL |
| SEQ ID NO: 468 | VL | QAVVTQEPSLTVSPGGTVTLTCRSSTGAVTTSNYAN |
| | | WVQQKPGQAPRGLIGGTNKRAPWTPARFSGSLLGD |
| | | KAALTLSGAQPEDEAEYFCALWYSNLWVFGCGTKL |
| | | TVL |
| SEQ ID NO: 469 | HCDR1 | GFTFNTYAMN |
| (Combined) | | |
| SEQ ID NO: 448 | HCDR2 | RIRSKYNNYATYYADSVKD |
| (Combined) | | |
| SEQ ID NO: 470 | HCDR3 | HGNFGNSYVSWFAY |
| (Combined) | | |
| SEQ ID NO: 471 | HCDR1 | TYAMN |
| (Kabat) | | |
| SEQ ID NO: 448 | HCDR2 | RIRSKYNNYATYYADSVKD |
| (Kabat) | | |
| SEQ ID NO: 470 | HCDR3 | HGNFGNSYVSWFAY |
| (Kabat) | | |
| SEQ ID NO: 472 | HCDR1 | GFTFNTY |
| (Chothia) | | |
| SEQ ID NO: 452 | HCDR2 | RSKYNNYA |
| (Chothia) | | |
| SEQ ID NO: 470 | HCDR3 | HGNFGNSYVSWFAY |
| (Chothia) | | |
| SEQ ID NO: 473 | HCDR1 | GFTFNTYA |
| (IMGT) | | |
| SEQ ID NO: 454 | HCDR2 | IRSKYNNYAT |
| (IMGT) | | |
| SEQ ID NO: 474 | HCDR3 | VRHGNFGNSYVSWFAY |
| (IMGT) | | |
| SEQ ID NO: 475 | VH | EVQLVESGGGLVQPGGSLKLSCAASGFTFNTYAMN |
| | | WVRQASGKGLEWVGRIRSKYNNYATYYADSVKDR |
| | | FTISRDDSKSTLYLQMNSLKTEDTAVYYCVRHGNFG |
| | | NSYVSWFAYWGQGTLVTVSS |
| SEQ ID NO: 476 | VH | EVQLVESGGGLVQPGGSLKLSCAASGFTFNTYAMN |
| | | WVRQASGKCLEWVGRIRSKYNNYATYYADSVKDR |
| | | FTISRDDSKSTLYLQMNSLKTEDTAVYYCVRHGNFG |
| | | NSYVSWFAYWGQGTLVTVSS |
| ANTI-CD3 (4) | | |
| SEQ ID NO: 477 | LCDR1 | RSSQSLVRSEGTTYFN |
| (Combined) | | |
| SEQ ID NO: 478 | LCDR2 | RVSNRFS |
| (Combined) | | |
| SEQ ID NO: 479 | LCDR3 | LQSSHFPWT |
| (Combined) | | |
| SEQ ID NO: 477 | LCDR1 | RSSQSLVRSEGTTYFN |
| (Kabat) | | |
| SEQ ID NO: 478 | LCDR2 | RVSNRFS |
| (Kabat) | | |
| SEQ ID NO: 479 | LCDR3 | LQSSHFPWT |
| (Kabat) | | |
| SEQ ID NO: 480 | LCDR1 | SQSLVRSEGTTY |
| (Chothia) | | |
| SEQ ID NO: 481 | LCDR2 | RVS |
| (Chothia) | | |
| SEQ ID NO: 482 | LCDR3 | SSHFPW |
| (Chothia) | | |
| SEQ ID NO: 483 | LCDR1 | QSLVRSEGTTY |
| (IMGT) | | |
| SEQ ID NO: 481 | LCDR2 | RVS |
| (IMGT) | | |
| SEQ ID NO: 479 | LCDR3 | LQSSHFPWT |
| (IMGT) | | |
| SEQ ID NO: 484 | VL | DILVTQTPVSLPVSLGGHVSISCRSSQSLVRSEGTTYF |
| | | NWYLQKPGQSPQLLIYRVSNRFSGVPDRFSGSGSGT |
| | | DFTLKISRVEPEDLGVYYCLQSSHFPWTFGGGTKLEL |
| | | K |
| SEQ ID NO: 485 | HCDR1 | GFTFSKQGMH |
| (Combined) | | |
| SEQ ID NO: 486 | HCDR2 | MIYYDSSKMYYADTVKG |
| (Combined) | | |
| SEQ ID NO: 487 | HCDR3 | FWWDLDFDH |
| (Combined) | | |
| SEQ ID NO: 488 | HCDR1 | KQGMH |
| (Kabat) | | |
| SEQ ID NO: 486 | HCDR2 | MIYYDSSKMYYADTVKG |
| (Kabat) | | |
| SEQ ID NO: 487 | HCDR3 | FWWDLDFDH |
| (Kabat) | | |
| SEQ ID NO: 489 | HCDR1 | GFTFSKQ |
| (Chothia) | | |
| SEQ ID NO: 490 | HCDR2 | YYDSSK |
| (Chothia) | | |
| SEQ ID NO: 487 | HCDR3 | FWWDLDFDH |
| (Chothia) | | |
| SEQ ID NO: 491 | HCDR1 | GFTFSKQG |
| (IMGT) | | |
| SEQ ID NO: 492 | HCDR2 | IYYDSSKM |
| (IMGT) | | |
| SEQ ID NO: 493 | HCDR3 | ASFWWDLDFDH |
| (IMGT) | | |
| SEQ ID NO: 494 | VH | EVKLVESGGDLVQPGDSLTLSCVASGFTFSKQGMH |
| | | WIRQAPKKGLEWIAMIYYDSSKMYYADTVKGRFTIS |
| | | RDNSKNTLYLEMNSLRSEDTAMYYCASFWWDLDF |
| | | DHWGQGVMVTVSS |
| ANTI-TCR | | |
| SEQ ID NO: 495 | LCDR1 | SATSSVSYMH |
| (Combined) | | |
| SEQ ID NO: 421 | LCDR2 | DTSKLAS |
| (Combined) | | |
| SEQ ID NO: 496 | LCDR3 | QQWSSNPLT |
| (Combined) | | |
| SEQ ID NO: 495 | LCDR1 | SATSSVSYMH |
| (Kabat) | | |
| SEQ ID NO: 421 | LCDR2 | DTSKLAS |
| (Kabat) | | |
| SEQ ID NO: 496 | LCDR3 | QQWSSNPLT |
| (Kabat) | | |
| SEQ ID NO: 497 | LCDR1 | TSSVSY |
| (Chothia) | | |
| SEQ ID NO: 424 | LCDR2 | DTS |
| (Chothia) | | |
| SEQ ID NO: 498 | LCDR3 | WSSNPL |
| (Chothia) | | |
| SEQ ID NO: 426 | LCDR1 | SSVSY |
| (IMGT) | | |
| SEQ ID NO: 424 | LCDR2 | DTS |
| (IMGT) | | |
| SEQ ID NO: 496 | LCDR3 | QQWSSNPLT |
| (IMGT) | | |
| SEQ ID NO: 499 | VL | QIVLTQSPAIMSASPGEKVTMTCSATSSVSYMHWYQ |
| | | QKSGTSPKRWIYDTSKLASGVPARFSGSGSGTSYSLT |
| | | ISSMEAEDAATYYCQQWSSNPLTFGAGTKLELK |
| SEQ ID NO: 500 | HCDR1 | GYKFTSYVMH |
| (Combined) | | |
| SEQ ID NO: 501 | HCDR2 | YINPYNDVTKYNEKFKG |
| (Combined) | | |
| SEQ ID NO: 502 | HCDR3 | GSYYDYDGFVY |
| (Combined) | | |
| SEQ ID NO: 503 | HCDR1 | SYVMH |
| (Kabat) | | |
| SEQ ID NO: 501 | HCDR2 | YINPYNDVTKYNEKFKG |
| (Kabat) | | |
| SEQ ID NO: 502 | HCDR3 | GSYYDYDGFVY |
| (Kabat) | | |
| SEQ ID NO: 504 | HCDR1 | GYKFTSY |
| (Chothia) | | |
| SEQ ID NO: 505 | HCDR2 | NPYNDV |
| (Chothia) | | |
| SEQ ID NO: 502 | HCDR3 | GSYYDYDGFVY |
| (Chothia) | | |
| SEQ ID NO: 506 | HCDR1 | GYKFTSYV |
| (IMGT) | | |
| SEQ ID NO: 507 | HCDR2 | INPYNDVT |
| (IMGT) | | |
| SEQ ID NO: 508 | HCDR3 | ARGSYYDYDGFVY |
| (IMGT) | | |
| SEQ ID NO: 509 | VH | EVQLQQSGPELVKPGASVKMSCKASGYKFTSYVMH |
| | | WVKQKPGQGLEWIGYINPYNDVTKYNEKFKGKATL |
| | | TSDKSSSTAYMELSSLTSEDSAVHYCARGSYYDYDG |
| | | FVYWGQGTLVTVSA |
| ANTI-CD28 (1) | | |
| SEQ ID NO: 510 | LCDR1 | HASQNIYVWLN |
| (Combined) | | |
| SEQ ID NO: 511 | LCDR2 | KASNLHT |
| (Combined) | | |
| SEQ ID NO: 512 | LCDR3 | QQGQTYPYT |
| (Combined) | | |
| SEQ ID NO: 510 | LCDR1 | HASQNIYVWLN |
| (Kabat) | | |
| SEQ ID NO: 511 | LCDR2 | KASNLHT |
| (Kabat) | | |
| SEQ ID NO: 512 | LCDR3 | QQGQTYPYT |
| (Kabat) | | |
| SEQ ID NO: 513 | LCDR1 | SQNIYVW |
| (Chothia) | | |
| SEQ ID NO: 514 | LCDR2 | KAS |
| (Chothia) | | |
| SEQ ID NO: 515 | LCDR3 | GQTYPY |
| (Chothia) | | |
| SEQ ID NO: 516 | LCDR1 | QNIYVW |
| (IMGT) | | |
| SEQ ID NO: 514 | LCDR2 | KAS |
| (IMGT) | | |
| SEQ ID NO: 512 | LCDR3 | QQGQTYPYT |
| (IMGT) | | |
| SEQ ID NO: 517 | VL | DIQMTQSPSSLSASVGDRVTITCHASQNIYVWLNWY |
| | | QQKPGKAPKLLIYKASNLHTGVPSRFSGSGSGTDFTL |
| | | TISSLQPEDFATYYCQQGQTYPYTFGGGTKVEIK |
| SEQ ID NO: 518 | LC | DIQMTQSPSSLSASVGDRVTITCHASQNIYVWLNWY |
| | | QQKPGKAPKLLIYKASNLHTGVPSRFSGSGSGTDFTL |
| | | TISSLQPEDFATYYCQQGQTYPYTFGGGTKVEIKRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |
| SEQ ID NO: 519 | HCDR1 | GYTFTSYYIH |
| (Combined) | | |
| SEQ ID NO: 520 | HCDR2 | CIYPGNVNTNYNEKFKD |
| (Combined) | | |
| SEQ ID NO: 521 | HCDR3 | SHYGLDWNFDV |
| (Combined) | | |
| SEQ ID NO: 522 | HCDR1 | SYYIH |
| (Kabat) | | |
| SEQ ID NO: 520 | HCDR2 | CIYPGNVNTNYNEKFKD |
| (Kabat) | | |
| SEQ ID NO: 521 | HCDR3 | SHYGLDWNFDV |
| (Kabat) | | |
| SEQ ID NO: 523 | HCDR1 | GYTFTSY |
| (Chothia) | | |
| SEQ ID NO: 524 | HCDR2 | YPGNVN |
| (Chothia) | | |
| SEQ ID NO: 521 | HCDR3 | SHYGLDWNFDV |
| (Chothia) | | |
| SEQ ID NO: 525 | HCDR1 | GYTFTSYY |
| (IMGT) | | |
| SEQ ID NO: 526 | HCDR2 | IYPGNVNT |
| (IMGT) | | |
| SEQ ID NO: 527 | HCDR3 | TRSHYGLDWNFDV |
| (IMGT) | | |
| SEQ ID NO: 528 | VH | QVQLVQSGAEVKKPGASVKVSCKASGYTFTSYYIH |
| | | WVRQAPGQGLEWIGCIYPGNVNTNYNEKFKDRATL |
| | | TVDTSISTAYMELSRLRSDDTAVYFCTRSHYGLDWN |
| | | FDVWGQGTTVTVSS |
| SEQ ID NO: 529 | HC | QVQLVQSGAEVKKPGASVKVSCKASGYTFTSYYIH |
| | | WVRQAPGQGLEWIGCIYPGNVNTNYNEKFKDRATL |
| | | TVDTSISTAYMELSRLRSDDTAVYFCTRSHYGLDWN |
| | | FDVWGQGTTVTVSSASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNHKPSNTKVD |
| | | KRVEPKSC |
| ANTI-CD28 (2) | | |
| SEQ ID NO: 530 | LCDR1 | SGSSSNIVSNYVN |
| (Combined) | | |
| SEQ ID NO: 531 | LCDR2 | DNNKRPS |
| (Combined) | | |
| SEQ ID NO: 532 | LCDR3 | QSYAIGSYSVV |
| (Combined) | | |
| SEQ ID NO: 530 | LCDR1 | SGSSSNIVSNYVN |
| (Kabat) | | |
| SEQ ID NO: 531 | LCDR2 | DNNKRPS |
| (Kabat) | | |
| SEQ ID NO: 532 | LCDR3 | QSYAIGSYSVV |
| (Kabat) | | |
| SEQ ID NO: 533 | LCDR1 | SSSNIVSNY |
| (Chothia) | | |
| SEQ ID NO: 534 | LCDR2 | DNN |
| (Chothia) | | |
| SEQ ID NO: 535 | LCDR3 | YAIGSYSV |
| (Chothia) | | |
| SEQ ID NO: 536 | LCDR1 | SSNIVSNY |
| (IMGT) | | |
| SEQ ID NO: 534 | LCDR2 | DNN |
| (IMGT) | | |
| SEQ ID NO: 532 | LCDR3 | QSYAIGSYSVV |
| (IMGT) | | |
| SEQ ID NO: 537 | VL | DIVLTQPPSVSGAPGQRVTISCSGSSSNIVSNYVNWY |
| | | QQLPGTAPKLLIYDNNKRPSGVPDRFSGSKSGTSASL |
| | | AITGLQSEDEADYYCQSYAIGSYSVVFGGGTKLTVL |
| SEQ ID NO: 538 | HCDR1 | GFTFSTYGMS |
| (Combined) | | |
| SEQ ID NO: 539 | HCDR2 | SIFYTGSSTYYADSVKG |
| (Combined) | | |
| SEQ ID NO: 540 | HCDR3 | IGYAGDSKYAI |
| (Combined) | | |
| SEQ ID NO: 541 | HCDR1 | TYGMS |
| (Kabat) | | |
| SEQ ID NO: 539 | HCDR2 | SIFYTGSSTYYADSVKG |
| (Kabat) | | |
| SEQ ID NO: 540 | HCDR3 | IGYAGDSKYAI |
| (Kabat) | | |
| SEQ ID NO: 542 | HCDR1 | GFTFSTY |
| (Chothia) | | |
| SEQ ID NO: 543 | HCDR2 | FYTGSS |
| (Chothia) | | |
| SEQ ID NO: 540 | HCDR3 | IGYAGDSKYAI |
| (Chothia) | | |
| SEQ ID NO: 544 | HCDR1 | GFTFSTYG |
| (IMGT) | | |
| SEQ ID NO: 545 | HCDR2 | IFYTGSST |
| (IMGT) | | |
| SEQ ID NO: 546 | HCDR3 | ARIGYAGDSKYAI |
| (IMGT) | | |
| SEQ ID NO: 547 | VH | QVQLVESGGGLVQPGGSLRLSCAASGFTFSTYGMS |
| | | WVRQAPGKGLEWVSSIFYTGSSTYYADSVKGRFTIS |
| | | RDNSKNTLYLQMNSLRAEDTAVYYCARIGYAGDSK |
| | | YAIWGQGTLVTVSS |
| SEQ ID NO: 548 | VH | EVQLVESGGGLVQPGGSLRLSCAASGFTFSTYGMSW |
| | | VRQAPGKGLEWVSSIFYTGSSTYYADSVKGRFTISRD |
| | | NSKNTLYLQMNSLRAEDTAVYYCARIGYAGDSKYA |
| | | IWGQGTLVTVSS |
| ANTI-ICOS | | |
| SEQ ID NO: 549 | LCDR1 | KSSQSLLSGSFNYLT |
| (Combined) | | |
| SEQ ID NO: 550 | LCDR2 | YASTRHT |
| (Combined) | | |
| SEQ ID NO: 551 | LCDR3 | HHHYNAPPT |
| (Combined) | | |
| SEQ ID NO: 549 | LCDR1 | KSSQSLLSGSFNYLT |
| (Kabat) | | |
| SEQ ID NO: 550 | LCDR2 | YASTRHT |
| (Kabat) | | |
| SEQ ID NO: 551 | LCDR3 | HHHYNAPPT |
| (Kabat) | | |
| SEQ ID NO: 552 | LCDR1 | SQSLLSGSFNY |
| (Chothia) | | |
| SEQ ID NO: 553 | LCDR2 | YAS |
| (Chothia) | | |
| SEQ ID NO: 554 | LCDR3 | HYNAPP |
| (Chothia) | | |
| SEQ ID NO: 555 | LCDR1 | QSLLSGSFNY |
| (IMGT) | | |
| SEQ ID NO: 553 | LCDR2 | YAS |
| (IMGT) | | |
| SEQ ID NO: 551 | LCDR3 | HHHYNAPPT |
| (IMGT) | | |
| SEQ ID NO: 556 | VL | DIVMTQSPDSLAVSLGERATINCKSSQSLLSGSFNYL |
| | | TWYQQKPGQPPKLLIFYASTRHTGVPDRFSGSGSGT |
| | | DFTLTISSLQAEDVAVYYCHHHYNAPPTFGPGTKVDI |
| | | K |
| SEQ ID NO: 557 | LC | DIVMTQSPDSLAVSLGERATINCKSSQSLLSGSFNYL |
| | | TWYQQKPGQPPKLLIFYASTRHTGVPDRFSGSGSGT |
| | | DFTLTISSLQAEDVAVYYCHHHYNAPPTFGPGTKVDI |
| | | KRTVAAPSVFIFPPSDEQLKSGTASVVCLLNNFYPRE |
| | | AKVQWKVDNALQSGNSQESVTEQDSKDSTYSLSST |
| | | LTLSKADYEKHKVYACEVTHQGLSSPVTKSFNRGEC |
| SEQ ID NO: 558 | HCDR1 | GFTFSDYWMD |
| (Combined) | | |
| SEQ ID NO: 559 | HCDR2 | NIDEDGSITEYSPFVKG |
| (Combined) | | |
| SEQ ID NO: 560 | HCDR3 | WGRFGFDS |
| (Combined) | | |
| SEQ ID NO: 561 | HCDR1 | DYWMD |
| (Kabat) | | |
| SEQ ID NO: 559 | HCDR2 | NIDEDGSITEYSPFVKG |
| (Kabat) | | |
| SEQ ID NO: 560 | HCDR3 | WGRFGFDS |
| (Kabat) | | |
| SEQ ID NO: 562 | HCDR1 | GFTFSDY |
| (Chothia) | | |
| SEQ ID NO: 563 | HCDR2 | DEDGSI |
| (Chothia) | | |
| SEQ ID NO: 560 | HCDR3 | WGRFGFDS |
| (Chothia) | | |
| SEQ ID NO: 564 | HCDR1 | GFTFSDYW |
| (IMGT) | | |
| SEQ ID NO: 565 | HCDR2 | IDEDGSIT |
| (IMGT) | | |
| SEQ ID NO: 566 | HCDR3 | TRWGRFGFDS |
| (IMGT) | | |
| SEQ ID NO: 567 | VH | EVQLVESGGGLVQPGGSLRLSCAASGFTFSDYWMD |
| | | WVRQAPGKGLVWVSNIDEDGSITEYSPFVKGRFTISR |
| | | DNAKNTLYLQMNSLRAEDTAVYYCTRWGRFGFDS |
| | | WGQGTLVTVSS |
| ANTI-CD27 | | |
| SEQ ID NO: 568 | LCDR1 | RASQGISRWLA |
| (Combined) | | |
| SEQ ID NO: 55 | LCDR2 | AASSLQS |
| (Combined) | | |
| SEQ ID NO: 569 | LCDR3 | QQYNTYPRT |
| (Combined) | | |
| SEQ ID NO: 568 | LCDR1 | RASQGISRWLA |
| (Kabat) | | |
| SEQ ID NO: 55 | LCDR2 | AASSLQS |
| (Kabat) | | |
| SEQ ID NO: 569 | LCDR3 | QQYNTYPRT |
| (Kabat) | | |
| SEQ ID NO: 570 | LCDR1 | SQGISRW |
| (Chothia) | | |
| SEQ ID NO: 58 | LCDR2 | AAS |
| (Chothia) | | |
| SEQ ID NO: 571 | LCDR3 | YNTYPR |
| (Chothia) | | |
| SEQ ID NO: 572 | LCDR1 | QGISRW |
| (IMGT) | | |
| SEQ ID NO: 58 | LCDR2 | AAS |
| (IMGT) | | |
| SEQ ID NO: 569 | LCDR3 | QQYNTYPRT |
| (IMGT) | | |
| SEQ ID NO: 573 | VL | DIQMTQSPSSLSASVGDRVTITCRASQGISRWLAWY |
| | | QQKPEKAPKSLIYAASSLQSGVPSRFSGSGSGTDFTL |
| | | TISSLQPEDFATYYCQQYNTYPRTFGQGTKVEIK |
| SEQ ID NO: 574 | HCDR1 | GFTFSSYDMH |
| (Combined) | | |
| SEQ ID NO: 575 | HCDR2 | VIWYDGSNKYYADSVKG |
| (Combined) | | |
| SEQ ID NO: 576 | HCDR3 | GSGNWGFFDY |
| (Combined) | | |
| SEQ ID NO: 577 | HCDR1 | SYDMH |
| (Kabat) | | |
| SEQ ID NO: 575 | HCDR2 | VIWYDGSNKYYADSVKG |
| (Kabat) | | |
| SEQ ID NO: 576 | HCDR3 | GSGNWGFFDY |
| (Kabat) | | |
| SEQ ID NO: 47 | HCDR1 | GFTFSSY |
| (Chothia) | | |
| SEQ ID NO: 578 | HCDR2 | WYDGSN |
| (Chothia) | | |
| SEQ ID NO: 576 | HCDR3 | GSGNWGFFDY |
| (Chothia) | | |
| SEQ ID NO: 579 | HCDR1 | GFTFSSYD |
| (IMGT) | | |
| SEQ ID NO: 580 | HCDR2 | IWYDGSNK |
| (IMGT) | | |
| SEQ ID NO: 581 | HCDR3 | ARGSGNWGFFDY |
| (IMGT) | | |
| SEQ ID NO: 582 | VH | QVQLVESGGGVVQPGRSLRLSCAASGFTFSSYDMH |
| | | WVRQAPGGLEWVAVIWYDGSNKYYADSVKGRFTIS |
| | | RDNSKNTLYLQMNSLRAEDTAVYYCARGSGNWGFF |
| | | DYWGQGTLVTVSS |
| ANTI-CD25 (1) | | |
| SEQ ID NO: 583 | LCDR1 | SASSSRSYMQ |
| (Combined) | | |
| SEQ ID NO: 421 | LCDR2 | DTSKLAS |
| (Combined) | | |
| SEQ ID NO: 584 | LCDR3 | HQRSSYT |
| (Combined) | | |
| SEQ ID NO: 583 | LCDR1 | SASSSRSYMQ |
| (Kabat) | | |
| SEQ ID NO: 421 | LCDR2 | DTSKLAS |
| (Kabat) | | |
| SEQ ID NO: 584 | LCDR3 | HQRSSYT |
| (Kabat) | | |
| SEQ ID NO: 585 | LCDR1 | SSSRSY |
| (Chothia) | | |
| SEQ ID NO: 424 | LCDR2 | DTS |
| (Chothia) | | |
| SEQ ID NO: 586 | LCDR3 | RSSY |
| (Chothia) | | |
| SEQ ID NO: 587 | LCDR1 | SSRSY |
| (IMGT) | | |
| SEQ ID NO: 424 | LCDR2 | DTS |
| (IMGT) | | |
| SEQ ID NO: 584 | LCDR3 | HQRSSYT |
| (IMGT) | | |
| SEQ ID NO: 588 | VL | QIVSTQSPAIMSASPGEKVTMTCSASSSRSYMQWYQ |
| | | QKPGTSPKRWIYDTSKLASGVPARFSGSGSGTSYSLT |
| | | ISSMEAEDAATYYCHQRSSYTFGGGTKLEIK |
| SEQ ID NO: 589 | HCDR1 | GYSFTRYWMH |
| (Combined) | | |
| SEQ ID NO: 590 | HCDR2 | AIYPGNSDTSYNQKFEG |
| (Combined) | | |
| SEQ ID NO: 591 | HCDR3 | DYGYYFDF |
| (Combined) | | |
| SEQ ID NO: 592 | HCDR1 | RYWMH |
| (Kabat) | | |
| SEQ ID NO: 590 | HCDR2 | AIYPGNSDTSYNQKFEG |
| (Kabat) | | |
| SEQ ID NO: 591 | HCDR3 | DYGYYFDF |
| (Kabat) | | |
| SEQ ID NO: 593 | HCDR1 | GYSFTRY |
| (Chothia) | | |
| SEQ ID NO: 594 | HCDR2 | YPGNSD |
| (Chothia) | | |
| SEQ ID NO: 591 | HCDR3 | DYGYYFDF |
| (Chothia) | | |
| SEQ ID NO: 595 | HCDR1 | GYSFTRYW |
| (IMGT) | | |
| SEQ ID NO: 596 | HCDR2 | IYPGNSDT |
| (IMGT) | | |
| SEQ ID NO: 597 | HCDR3 | SRDYGYYFDF |
| (IMGT) | | |
| SEQ ID NO: 598 | VH | EVQLQQSGTVLARPGASVKMSCKASGYSFTRYWMH |
| | | WIKQRPGQGLEWIGAIYPGNSDTSYNQKFEGKAKLT |
| | | AVTSASTAYMELSSLTHEDSAVYYCSRDYGYYFDF |
| | | WGQGTTLTVSS |
| ANTI-CD25 (2) | | |
| SEQ ID NO: 599 | LCDR1 | KASQSVDYDGDSYMN |
| (Combined) | | |
| SEQ ID NO: 600 | LCDR2 | AASNLES |
| (Combined) | | |
| SEQ ID NO: 601 | LCDR3 | QQSNEDPYT |
| (Combined) | | |
| SEQ ID NO: 599 | LCDR1 | KASQSVDYDGDSYMN |
| (Kabat) | | |
| SEQ ID NO: 600 | LCDR2 | AASNLES |
| (Kabat) | | |
| SEQ ID NO: 601 | LCDR3 | QQSNEDPYT |
| (Kabat) | | |
| SEQ ID NO: 602 | LCDR1 | SQSVDYDGDSY |
| (Chothia) | | |
| SEQ ID NO: 58 | LCDR2 | AAS |
| (Chothia) | | |
| SEQ ID NO: 603 | LCDR3 | SNEDPY |
| (Chothia) | | |
| SEQ ID NO: 604 | LCDR1 | QSVDYDGDSY |
| (IMGT) | | |
| SEQ ID NO: 58 | LCDR2 | AAS |
| (IMGT) | | |
| SEQ ID NO: 601 | LCDR3 | QQSNEDPYT |
| (IMGT) | | |
| SEQ ID NO: 605 | VL | DIVLTQSPLSLPVTLGQPASISCKASQSVDYDGDSYM |
| | | NWYQQRPGQSPRLLIYAASNLESGVPDRFSGSGSGT |
| | | DFTLKISRVEAEDVGVYYCQQSNEDPYTFGGGTKVE |
| | | IK |
| SEQ ID NO: 606 | LC | DIVLTQSPLSLPVTLGQPASISCKASQSVDYDGDSYM |
| | | NWYQQRPGQSPRLLIYAASNLESGVPDRFSGSGSGT |
| | | DFTLKISRVEAEDVGVYYCQQSNEDPYTFGGGTKVE |
| | | IKRTVAAPSVFIFPPSDEQLKSGTASVVCLLNNFYPRE |
| | | AKVQWKVDNALQSGNSQESVTEQDSKDSTYSLSST |
| | | LTLSKADYEKHKVYACEVTHQGLSSPVTKSFNRGEC |
| SEQ ID NO: 607 | HCDR1 | GYAFTNYLIE |
| (Combined) | | |
| SEQ ID NO: 608 | HCDR2 | VINPGSGGTNYNEKFKG |
| (Combined) | | |
| SEQ ID NO: 609 | HCDR3 | WRGDGYYAYFDV |
| (Combined) | | |
| SEQ ID NO: 610 | HCDR1 | NYLIE |
| (Kabat) | | |
| SEQ ID NO: 608 | HCDR2 | VINPGSGGTNYNEKFKG |
| (Kabat) | | |
| SEQ ID NO: 609 | HCDR3 | WRGDGYYAYFDV |
| (Kabat) | | |
| SEQ ID NO: 611 | HCDR1 | GYAFTNY |
| (Chothia) | | |
| SEQ ID NO: 612 | HCDR2 | NPGSGG |
| (Chothia) | | |
| SEQ ID NO: 609 | HCDR3 | WRGDGYYAYFDV |
| (Chothia) | | |
| SEQ ID NO: 613 | HCDR1 | GYAFTNYL |
| (IMGT) | | |
| SEQ ID NO: 614 | HCDR2 | INPGSGGT |
| (IMGT) | | |
| SEQ ID NO: 615 | HCDR3 | ARWRGDGYYAYFDV |
| (IMGT) | | |
| SEQ ID NO: 616 | VH | EVQLVQSGAEVKKPGESLKISCKGSGYAFTNYLIEW |
| | | VRQMPGKGLEWMGVINPGSGGTNYNEKFKGQVTIS |
| | | ADKSISTAYLQWSSLKASDTAMYYCARWRGDGYY |
| | | AYFDVWGQGTTVTVSS |
| SEQ ID NO: 617 | HC | EVQLVQSGAEVKKPGESLKISCKGSGYAFTNYLIEW |
| | | VRQMPGKGLEWMGVINPGSGGTNYNEKFKGQVTIS |
| | | ADKSISTAYLQWSSLKASDTAMYYCARWRGDGYY |
| | | AYFDVWGQGTTVTVSSASTKGPSVFPLAPSSKSTSG |
| | | GTAALGCLVKDYFPEPVTVSWNSGALTSGVHTFPAV |
| | | LQSSGLYSLSSVVTVPSSSLGTQTYICNVNHKPSNTK |
| | | VDKRVEPKSC |
| ANTI-4-1BB | | |
| SEQ ID NO: 618 | LCDR1 | RASQSVSSYLA |
| (Combined) | | |
| SEQ ID NO: 619 | LCDR2 | DASNRAT |
| (Combined) | | |
| SEQ ID NO: 620 | LCDR3 | QQRSNWPPALT |
| (Combined) | | |
| SEQ ID NO: 618 | LCDR1 | RASQSVSSYLA |
| (Kabat) | | |
| SEQ ID NO: 619 | LCDR2 | DASNRAT |
| (Kabat) | | |
| SEQ ID NO: 620 | LCDR3 | QQRSNWPPALT |
| (Kabat) | | |
| SEQ ID NO: 621 | LCDR1 | SQSVSSY |
| (Chothia) | | |
| SEQ ID NO: 622 | LCDR2 | DAS |
| (Chothia) | | |
| SEQ ID NO: 623 | LCDR3 | RSNWPPAL |
| (Chothia) | | |
| SEQ ID NO: 624 | LCDR1 | QSVSSY |
| (IMGT) | | |
| SEQ ID NO: 622 | LCDR2 | DAS |
| (IMGT) | | |
| SEQ ID NO: 620 | LCDR3 | QQRSNWPPALT |
| (IMGT) | | |
| SEQ ID NO: 625 | VL | EIVLTQSPATLSLSPGERATLSCRASQSVSSYLAWYQ |
| | | QKPGQAPRLLIYDASNRATGIPARFSGSGSGTDFTLTI |
| | | SSLEPEDFAVYYCQQRSNWPPALTFCGGTKVEIK |
| SEQ ID NO: 626 | HCDR1 | GGSFSGYYWS |
| (Combined) | | |
| SEQ ID NO: 627 | HCDR2 | EINHGGYVTYNPSLES |
| (Combined) | | |
| SEQ ID NO: 628 | HCDR3 | DYGPGNYDWYFDL |
| (Combined) | | |
| SEQ ID NO: 629 | HCDR1 | GYYWS |
| (Kabat) | | |
| SEQ ID NO: 627 | HCDR2 | EINHGGYVTYNPSLES |
| (Kabat) | | |
| SEQ ID NO: 628 | HCDR3 | DYGPGNYDWYFDL |
| (Kabat) | | |
| SEQ ID NO: 630 | HCDR1 | GGSFSGY |
| (Chothia) | | |
| SEQ ID NO: 631 | HCDR2 | NHGGY |
| (Chothia) | | |
| SEQ ID NO: 628 | HCDR3 | DYGPGNYDWYFDL |
| (Chothia) | | |
| SEQ ID NO: 632 | HCDR1 | GGSFSGYY |
| (IMGT) | | |
| SEQ ID NO: 633 | HCDR2 | INHGGYV |
| (IMGT) | | |
| SEQ ID NO: 634 | HCDR3 | ARDYGPGNYDWYFDL |
| (IMGT) | | |
| SEQ ID NO: 635 | VH | QVQLQQWGAGLLKPSETLSLTCAVYGGSFSGYYWS |
| | | WIRQSPEKGLEWIGEINHGGYVTYNPSLESRVTISVD |
| | | TSKNQFSLKLSSVTAADTAVYYCARDYGPGNYDWY |
| | | FDLWGRGTLVTVSS |
| ANTI-IL6RA | | |
| SEQ ID NO: 636 | LCDR1 | RASQDISSYLN |
| (Combined) | | |
| SEQ ID NO: 637 | LCDR2 | YTSRLHS |
| (Combined) | | |
| SEQ ID NO: 300 | LCDR3 | QQGNTLPYT |
| (Combined) | | |
| SEQ ID NO: 636 | LCDR1 | RASQDISSYLN |
| (Kabat) | | |
| SEQ ID NO: 637 | LCDR2 | YTSRLHS |
| (Kabat) | | |
| SEQ ID NO: 300 | LCDR3 | QQGNTLPYT |
| (Kabat) | | |
| SEQ ID NO: 638 | LCDR1 | SQDISSY |
| (Chothia) | | |
| SEQ ID NO: 639 | LCDR2 | YTS |
| (Chothia) | | |
| SEQ ID NO: 640 | LCDR3 | GNTLPY |
| (Chothia) | | |
| SEQ ID NO: 641 | LCDR1 | QDISSY |
| (IMGT) | | |
| SEQ ID NO: 639 | LCDR2 | YTS |
| (IMGT) | | |
| SEQ ID NO: 300 | LCDR3 | QQGNTLPYT |
| (IMGT) | | |
| SEQ ID NO: 642 | VL | DIQMTQSPSSLSASVGDRVTITCRASQDISSYLNWYQ |
| | | QKPGKAPKLLIYYTSRLHSGVPSRFSGSGSGTDFTFTI |
| | | SSLQPEDIATYYCQQGNTLPYTFGQGTKVEIK |
| SEQ ID NO: 643 | HCDR1 | GYSITSDHAWS |
| (Combined) | | |
| SEQ ID NO: 644 | HCDR2 | YISYSGITTYNPSLKS |
| (Combined) | | |
| SEQ ID NO: 645 | HCDR3 | SLARTTAMDY |
| (Combined) | | |
| SEQ ID NO: 646 | HCDR1 | SDHAWS |
| (Kabat) | | |
| SEQ ID NO: 644 | HCDR2 | YISYSGITTYNPSLKS |
| (Kabat) | | |
| SEQ ID NO: 645 | HCDR3 | SLARTTAMDY |
| (Kabat) | | |
| SEQ ID NO: 647 | HCDR1 | GYSITSDH |
| (Chothia) | | |
| SEQ ID NO: 648 | HCDR2 | SYSGI |
| (Chothia) | | |
| SEQ ID NO: 645 | HCDR3 | SLARTTAMDY |
| (Chothia) | | |
| SEQ ID NO: 649 | HCDR1 | GYSITSDHA |
| (IMGT) | | |
| SEQ ID NO: 650 | HCDR2 | ISYSGIT |
| (IMGT) | | |
| SEQ ID NO: 651 | HCDR3 | ARSLARTTAMDY |
| (IMGT) | | |
| SEQ ID NO: 652 | VH | QVQLQESGPGLVRPSQTLSLTCTVSGYSITSDHAWS |
| | | WVRQPPGRGLEWIGYISYSGITTYNPSLKSRVTMLR |
| | | DTSKNQFSLRLSSVTAADTAVYYCARSLARTTAMD |
| | | YWGQGSLVTVSS |
| ANTI-IL6RB | | |
| SEQ ID NO: 54 | LCDR1 | RASQSISSYLN |
| (Combined) | | |
| SEQ ID NO: 55 | LCDR2 | AASSLQS |
| (Combined) | | |
| SEQ ID NO: 653 | LCDR3 | QQSYSTPPIT |
| (Combined) | | |
| SEQ ID NO: 54 | LCDR1 | RASQSISSYLN |
| (Kabat) | | |
| SEQ ID NO: 55 | LCDR2 | AASSLQS |
| (Kabat) | | |
| SEQ ID NO: 653 | LCDR3 | QQSYSTPPIT |
| (Kabat) | | |
| SEQ ID NO: 57 | LCDR1 | SQSISSY |
| (Chothia) | | |
| SEQ ID NO: 58 | LCDR2 | AAS |
| (Chothia) | | |
| SEQ ID NO: 654 | LCDR3 | SYSTPPI |
| (Chothia) | | |
| SEQ ID NO: 60 | LCDR1 | QSISSY |
| (IMGT) | | |
| SEQ ID NO: 58 | LCDR2 | AAS |
| (IMGT) | | |
| SEQ ID NO: 653 | LCDR3 | QQSYSTPPIT |
| (IMGT) | | |
| SEQ ID NO: 655 | VL | DIQMTQSPSSLSASVGDRVTITCRASQSISSYLNWYQ |
| | | QKPGKAPKLLIYAASSLQSGVPSRFSGSGSGTDFTLTI |
| | | SSLQPEDFATYYCQQSYSTPPITFGQGTRLEIK |
| SEQ ID NO: 656 | HCDR1 | GFTFSDYYMT |
| (Combined) | | |
| SEQ ID NO: 657 | HCDR2 | YISSSGTNKYNADSVKG |
| (Combined) | | |
| SEQ ID NO: 658 | HCDR3 | DPPWGMDV |
| (Combined) | | |
| SEQ ID NO: 659 | HCDR1 | DYYMT |
| (Kabat) | | |
| SEQ ID NO: 657 | HCDR2 | YISSSGTNKYNADSVKG |
| (Kabat) | | |
| SEQ ID NO: 658 | HCDR3 | DPPWGMDV |
| (Kabat) | | |
| SEQ ID NO: 562 | HCDR1 | GFTFSDY |
| (Chothia) | | |
| SEQ ID NO: 660 | HCDR2 | SSSGTN |
| (Chothia) | | |
| SEQ ID NO: 658 | HCDR3 | DPPWGMDV |
| (Chothia) | | |
| SEQ ID NO: 661 | HCDR1 | GFTFSDYY |
| (IMGT) | | |
| SEQ ID NO: 662 | HCDR2 | ISSSGTNK |
| (IMGT) | | |
| SEQ ID NO: 663 | HCDR3 | VRDPPWGMDV |
| (IMGT) | | |
| SEQ ID NO: 664 | VH | QVQLVESGGGLVKPGGSLRLSCAASGFTFSDYYMT |
| | | WIRQTPGKGLDWVSYISSSGTNKYNADSVKGRFTIS |
| | | RDNAKNSLYLQMNSLRAEDTAVYYCVRDPPWGMD |
| | | VWGQGTTVTVSS |
| ANTI-CD2 (1) | | |
| SEQ ID NO: 665 | LCDR1 | RSSQSLLHSSGNTYLN |
| (Combined) | | |
| SEQ ID NO: 666 | LCDR2 | LVSKLES |
| (Combined) | | |
| SEQ ID NO: 667 | LCDR3 | MQFTHYPYT |
| (Combined) | | |
| SEQ ID NO: 665 | LCDR1 | RSSQSLLHSSGNTYLN |
| (Kabat) | | |
| SEQ ID NO: 666 | LCDR2 | LVSKLES |
| (Kabat) | | |
| SEQ ID NO: 667 | LCDR3 | MQFTHYPYT |
| (Kabat) | | |
| SEQ ID NO: 668 | LCDR1 | SQSLLHSSGNTY |
| (Chothia) | | |
| SEQ ID NO: 669 | LCDR2 | LVS |
| (Chothia) | | |
| SEQ ID NO: 670 | LCDR3 | FTHYPY |
| (Chothia) | | |
| SEQ ID NO: 671 | LCDR1 | QSLLHSSGNTY |
| (IMGT) | | |
| SEQ ID NO: 669 | LCDR2 | LVS |
| (IMGT) | | |
| SEQ ID NO: 667 | LCDR3 | MQFTHYPYT |
| (IMGT) | | |
| SEQ ID NO: 672 | VL | DVVMTQSPPSLLVTLGQPASISCRSSQSLLHSSGNTY |
| | | LNWLLQRPGQSPQPLIYLVSKLESGVPDRFSGSGSGT |
| | | DFTLKISGVEAEDVGVYYCMQFTHYPYTFGQGTKLE |
| | | IK |
| SEQ ID NO: 673 | LC | DVVMTQSPPSLLVTLGQPASISCRSSQSLLHSSGNTY |
| | | LNWLLQRPGQSPQPLIYLVSKLESGVPDRFSGSGSGT |
| | | DFTLKISGVEAEDVGVYYCMQFTHYPYTFGQGTKLE |
| | | IKRTVAAPSVFIFPPSDEQLKSGTASVVCLLNNFYPRE |
| | | AKVQWKVDNALQSGNSQESVTEQDSKDSTYSLSST |
| | | LTLSKADYEKHKVYACEVTHQGLSSPVTKSFNRGEC |
| SEQ ID NO: 674 | HCDR1 | GYIFTEYYMY |
| (Combined) | | |
| SEQ ID NO: 675 | HCDR2 | RIDPEDGSIDYVEKFKK |
| (Combined) | | |
| SEQ ID NO: 676 | HCDR3 | GKFNYRFAY |
| (Combined) | | |
| SEQ ID NO: 677 | HCDR1 | EYYMY |
| (Kabat) | | |
| SEQ ID NO: 675 | HCDR2 | RIDPEDGSIDYVEKFKK |
| (Kabat) | | |
| SEQ ID NO: 676 | HCDR3 | GKFNYRFAY |
| (Kabat) | | |
| SEQ ID NO: 678 | HCDR1 | GYIFTEY |
| (Chothia) | | |
| SEQ ID NO: 679 | HCDR2 | DPEDGS |
| (Chothia) | | |
| SEQ ID NO: 676 | HCDR3 | GKFNYRFAY |
| (Chothia) | | |
| SEQ ID NO: 680 | HCDR1 | GYIFTEYY |
| (IMGT) | | |
| SEQ ID NO: 681 | HCDR2 | IDPEDGSI |
| (IMGT) | | |
| SEQ ID NO: 682 | HCDR3 | ARGKFNYRFAY |
| (IMGT) | | |
| SEQ ID NO: 683 | VH | QVQLVQSGAEVQRPGASVKVSCKASGYIFTEYYMY |
| | | WVRQAPGQGLELVGRIDPEDGSIDYVEKFKKKVTLT |
| | | ADTSSSTAYMELSSLTSDDTAVYYCARGKFNYRFAY |
| | | WGQGTLVTVSS |
| SEQ ID NO: 684 | HC | QVQLVQSGAEVQRPGASVKVSCKASGYIFTEYYMY |
| | | WVRQAPGQGLELVGRIDPEDGSIDYVEKFKKKVTLT |
| | | ADTSSSTAYMELSSLTSDDTAVYYCARGKFNYRFAY |
| | | WGQGTLVTVSSASTKGPSVFPLAPSSKSTSGGTAAL |
| | | GCLVKDYFPEPVTVSWNSGALTSGVHTFPAVLQSSG |
| | | LYSLSSVVTVPSSSLGTQTYICNVNHKPSNTKVDKK |
| | | VEPKSC |

In some embodiments, the agent that stimulates a CD3/TCR complex and the agent that stimulates a costimulatory molecule and/or growth factor receptor are comprised in a multispecific binding molecule. Thus, contemplated herein are multispecific binding molecules comprising an agent that stimulates a CD3/TCR complex and an agent that stimulates a costimulatory molecule and/or growth factor receptor, such as but not limited to a multispecific binding molecule comprising a CD3 antigen binding domain and one or more of a CD28, ICOS, CD27, CD25, 4-1BB, IL6RA, IL6RB, and/or CD2 antigen binding domain. Non-limiting examples of such binding domains, as noted above, are provided above, for example in Table 27 and the publications incorporated by reference herein.

In some embodiments, the multispecific binding molecule comprises a CD3 antigen binding domain and a CD28 or CD2 antigen binding domain. In some embodiments, the CD3 antigen binding domain is an anti-CD3 antibody, optionally the anti-CD3 (1), anti-CD3 (2), anti-CD3 (3), or anti-CD3 (4) provided in Table 27, or an antibody fragment comprising one or more CDRs, VH, and/or VL thereof. In some embodiments, the CD28 antigen binding domain is an anti-CD28 antibody, optionally the anti-CD28 (1) or anti-CD28 (2) provided in Table 27, or an antibody fragment comprising one or more CDRs, VH, heavy chain, VL, and/or light chain thereof. In some embodiments, the CD2 antigen binding domain is an anti-CD2 antibody, optionally the anti-CD2 (1), provided in Table 27, or an antibody fragment comprising one or more CDRs, VH, heavy chain, VL, and/or light chain thereof.

In some embodiments, the multispecific binding molecules comprise one or more heavy and/or light chains. Non-limiting exemplary heavy and light chain sequences that may be comprised in these multispecific binding molecules are provided in Table 28 below. Non-limiting exemplary combinations thereof are suggested in Table 28 based on the categorization of the recited heavy and/or light chains as within a Construct. This Construct organization provides examples of configurations of heavy and/or light chains but further combinations and permutations thereof are also possible.

In some embodiments, an Anti-CD3 (4)/anti-CD28 (2) bispecific construct is also referred to as Construct A herein. In some embodiments, an Anti-CD3 (2)/anti-CD28 (2) construct is referred to as Construct B herein. In some embodiments, an Anti-CD3 (4)/anti-CD28 (1) construct is referred to as Construct C herein.

TABLE 28
Exemplary Fc, heavy chain (HC), and light chain (LC) sequences

| SEQ ID | | |
|---|---|---|
| No | Chain | Amino acid sequence |
| 701 | Wild type Fc | DKTHTCPPCPAPELLGGPSVFLFPPKPKDTLMISRTPEVTCVVV |
| | | DVSHEDPEVKFNWYVDGVEVHNAKTKPREEQYNSTYRVVSV |
| | | LTVLHQDWLNGKEYKCKVSNKALPAPIEKTISKAKGQPREPQV |
| | | YTLPPSREEMTKNQVSLTCLVKGFYPSDIAVEWESNGQPENNY |
| | | KTTPPVLDSDGSFFLYSKLTVDKSRWQQGNVFSCSVMHEALH |
| | | NHYTQKSLSLSPGK |
| 702 | Fc DANAPA | DKTHTCPPCPAPELLGGPSVFLFPPKPKDTLMISRTPEVTCVVV |
| | | AVSHEDPEVKFNWYVDGVEVHNAKTKPREEQYASTYRVVSV |
| | | LTVLHQDWLNGKEYKCKVSNKALAAPIEKTISKAKGQPREPQ |
| | | VYTLPPSREEMTKNQVSLTCLVKGFYPSDIAVEWESNGQPENN |
| | | YKTTPPVLDSDGSFFLYSKLTVDKSRWQQGNVFSCSVMHEAL |
| | | HNHYTQKSLSLSPGK |
| 673 | NEG2042 LC | DVVMTQSPPSLLVTLGQPASISCRSSQSLLHSSGNTYLNWLLQR |
| | | PGQSPQPLIYLVSKLESGVPDRFSGSGSGTDFTLKISGVEAEDV |
| | | GVYYCMQFTHYPYTFGQGTKLEIKRTVAAPSVFIFPPSDEQLKS |
| | | GTASVVCLLNNFYPREAKVQWKVDNALQSGNSQESVTEQDSK |
| | | DSTYSLSSTLTLSKADYEKHKVYACEVTHQGLSSPVTKSFNRG |
| | | EC |
| 703 | NEG2042 HC | QVQLVQSGAEVQRPGASVKVSCKASGYIFTEYYMYWVRQAP |
| | | GQGLELVGRIDPEDGSIDYVEKFKKKVTLTADTSSSTAYMELSS |
| | | LTSDDTAVYYCARGKFNYRFAYWGQGTLVTVSSASTKGPSVF |
| | | PLAPSSKSTSGGTAALGCLVKDYFPEPVTVSWNSGALTSGVHT |
| | | FPAVLQSSGLYSLSSVVTVPSSSLGTQTYICNVNHKPSNTKVDK |
| | | RVEPKSCDKTHTCPPCPAPELLGGPSVFLFPPKPKDTLMISRTPE |
| | | VTCVVVAVSHEDPEVKFNWYVDGVEVHNAKTKPREEQYAST |
| | | YRVVSVLTVLHQDWLNGKEYKCKVSNKALAAPIEKTISKAKG |
| | | QPREPQVYTLPPSREEMTKNQVSLTCLVKGFYPSDIAVEWESN |
| | | GQPENNYKTTPPVLDSDGSFFLYSKLTVDKSRWQQGNVFSCSV |
| | | MHEALHNHYTQKSLSLSPGGGGSGGGGSGGGGSGGGGSQVQ |
| | | LVQSGGGVVQPGRSLRLSCKASGYTFTRYTMHWVRQAPGKG |
| | | LEWIGYINPSRGYTNYNQKVKDRFTISRDNSKNTAFLQMDSLR |
| | | PEDTGVYFCARYYDDHYSLDYWGQGTPVTVSSGGGGSGGGG |
| | | SGGGGSGGGGSDIQMTQSPSSLSASVGDRVTITCSASSSVSYMN |
| | | WYQQTPGKAPKRWIYDTSKLASGVPSRFSGSGSGTDYTFTISSL |
| | | QPEDIATYYCQQWSSNPFTFGQGTKLQIT |
| 518 | NEG2043 LC | DIQMTQSPSSLSASVGDRVTITCHASQNIYVWLNWYQQKPGK |
| | | APKLLIYKASNLHTGVPSRFSGSGSGTDFTLTISSLQPEDFATYY |
| | | CQQGQTYPYTFGGGTKVEIKRTVAAPSVFIFPPSDEQLKSGTAS |
| | | VVCLLNNFYPREAKVQWKVDNALQSGNSQESVTEQDSKDSTY |
| | | SLSSTLTLSKADYEKHKVYACEVTHQGLSSPVTKSFNRGEC |
| 704 | NEG2043 HC | QVQLVQSGAEVKKPGASVKVSCKASGYTFTSYYIHWVRQAPG |
| | | QGLEWIGCIYPGNVNTNYNEKFKDRATLTVDTSISTAYMELSR |
| | | LRSDDTAVYFCTRSHYGLDWNFDVWGQGTTVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRVEPKSCDKTHTCPPCPAPELLGGPSVFLFPPKPKDTLMISR |
| | | TPEVTCVVVAVSHEDPEVKFNWYVDGVEVHNAKTKPREEQY |
| | | ASTYRVVSVLTVLHQDWLNGKEYKCKVSNKALAAPIEKTISK |
| | | AKGQPREPQVYTLPPSREEMTKNQVSLTCLVKGFYPSDIAVEW |
| | | ESNGQPENNYKTTPPVLDSDGSFFLYSKLTVDKSRWQQGNVFS |
| | | CSVMHEALHNHYTQKSLSLSPGGGGSGGGGSGGGGSGGGGSQ |
| | | VQLVQSGGGVVQPGRSLRLSCKASGYTFTRYTMHWVRQAPG |
| | | KGLEWIGYINPSRGYTNYNQKVKDRFTISRDNSKNTAFLQMDS |
| | | LRPEDTGVYFCARYYDDHYSLDYWGQGTPVTVSSGGGGSGG |
| | | GGSGGGGSGGGGSDIQMTQSPSSLSASVGDRVTITCSASSSVSY |
| | | MNWYQQTPGKAPKRWIYDTSKLASGVPSRFSGSGSGTDYTFTI |
| | | SSLQPEDIATYYCQQWSSNPFTFGQGTKLQIT |
| 705 | NEG2044 LC | DIVLTQPPSVSGAPGQRVTISCSGSSSNIVSNYVNWYQQLPGTA |
| | | PKLLIYDNNKRPSGVPDRFSGSKSGTSASLAITGLQSEDEADYY |
| | | CQSYAIGSYSVVFGGGTKLTVLGQGTKVEIKRTVAAPSVFIFPP |
| | | SDEQLKSGTASVVCLLNNFYPREAKVQWKVDNALQSGNSQES |
| | | VTEQDSKDSTYSLSSTLTLSKADYEKHKVYACEVTHQGLSSPV |
| | | TKSFNRGEC |
| 706 | NEG2044 HC | QVQLVESGGGLVQPGGSLRLSCAASGFTFSTYGMSWVRQAPG |
| | | KGLEWVSSIFYTGSSTYYADSVKGRFTISRDNSKNTLYLQMNS |
| | | LRAEDTAVYYCARIGYAGDSKYAIWGQGTLVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRVEPKSCDKTHTCPPCPAPELLGGPSVFLFPPKPKDTLMISR |
| | | TPEVTCVVVAVSHEDPEVKFNWYVDGVEVHNAKTKPREEQY |
| | | ASTYRVVSVLTVLHQDWLNGKEYKCKVSNKALAAPIEKTISK |
| | | AKGQPREPQVYTLPPSREEMTKNQVSLTCLVKGFYPSDIAVEW |
| | | ESNGQPENNYKTTPPVLDSDGSFFLYSKLTVDKSRWQQGNVFS |
| | | CSVMHEALHNHYTQKSLSLSPGGGGSGGGGSGGGGSGGGGSQ |
| | | VQLVQSGGGVVQPGRSLRLSCKASGYTFTRYTMHWVRQAPG |
| | | KGLEWIGYINPSRGYTNYNQKVKDRFTISRDNSKNTAFLQMDS |
| | | LRPEDTGVYFCARYYDDHYSLDYWGQGTPVTVSSGGGGSGG |
| | | GGSGGGGSGGGGSDIQMTQSPSSLSASVGDRVTITCSASSSVSY |
| | | MNWYQQTPGKAPKRWIYDTSKLASGVPSRFSGSGSGTDYTFTI |
| | | SSLQPEDIATYYCQQWSSNPFTFGQGTKLQIT |
| 673 | NEG2050 LC | DVVMTQSPPSLLVTLGQPASISCRSSQSLLHSSGNTYLNWLLQR |
| | | PGQSPQPLIYLVSKLESGVPDRFSGSGSGTDFTLKISGVEAEDV |
| | | GVYYCMQFTHYPYTFGQGTKLEIKRTVAAPSVFIFPPSDEQLKS |
| | | GTASVVCLLNNFYPREAKVQWKVDNALQSGNSQESVTEQDSK |
| | | DSTYSLSSTLTLSKADYEKHKVYACEVTHQGLSSPVTKSFNRG |
| | | EC |
| 707 | NEG2050 HC | QVQLVQSGAEVQRPGASVKVSCKASGYIFTEYYMYWVRQAP |
| | | GQGLELVGRIDPEDGSIDYVEKFKKKVTLTADTSSSTAYMELSS |
| | | LTSDDTAVYYCARGKFNYRFAYWGQGTLVTVSSASTKGPSVF |
| | | PLAPSSKSTSGGTAALGCLVKDYFPEPVTVSWNSGALTSGVHT |
| | | FPAVLQSSGLYSLSSVVTVPSSSLGTQTYICNVNHKPSNTKVDK |
| | | RVEPKSCDKTHTCPPCPAPELLGGPSVFLFPPKPKDTLMISRTPE |
| | | VTCVVVAVSHEDPEVKFNWYVDGVEVHNAKTKPREEQYAST |
| | | YRVVSVLTVLHQDWLNGKEYKCKVSNKALAAPIEKTISKAKG |
| | | QPREPQVYTLPPSREEMTKNQVSLTCLVKGFYPSDIAVEWESN |
| | | GQPENNYKTTPPVLDSDGSFFLYSKLTVDKSRWQQGNVFSCSV |
| | | MHEALHNHYTQKSLSLSPGGGGSGGGGSGGGGSGGGGSEVQL |
| | | VESGGGLVQPGGSLKLSCAASGFTFNKYAMNWVRQAPGKCLE |
| | | WVARIRSKYNNYATYYADSVKDRFTISRDDSKNTAYLQMNNL |
| | | KTEDTAVYYCVRHGNFGNSYISYWAYWGQGTLVTVSSGGGG |
| | | SGGGGSGGGGSGGGGSQTVVTQEPSLTVSPGGTVTLTCGSSTG |
| | | AVTSGNYPNWVQQKPGQAPRGLIGGTKFLAPGTPARFSGSLLG |
| | | GKAALTLSGVQPEDEAEYYCVLWYSNRWVFGCGTKLTVL |
| 673 | NEG2051 LC | DVVMTQSPPSLLVTLGQPASISCRSSQSLLHSSGNTYLNWLLQR |
| | | PGQSPQPLIYLVSKLESGVPDRFSGSGSGTDFTLKISGVEAEDV |
| | | GVYYCMQFTHYPYTFGQGTKLEIKRTVAAPSVFIFPPSDEQLKS |
| | | GTASVVCLLNNFYPREAKVQWKVDNALQSGNSQESVTEQDSK |
| | | DSTYSLSSTLTLSKADYEKHKVYACEVTHQGLSSPVTKSFNRG |
| | | EC |
| 708 | NEG2051 HC | QVQLVQSGAEVQRPGASVKVSCKASGYIFTEYYMYWVRQAP |
| | | GQGLELVGRIDPEDGSIDYVEKFKKKVTLTADTSSSTAYMELSS |
| | | LTSDDTAVYYCARGKFNYRFAYWGQGTLVTVSSASTKGPSVF |
| | | PLAPSSKSTSGGTAALGCLVKDYFPEPVTVSWNSGALTSGVHT |
| | | FPAVLQSSGLYSLSSVVTVPSSSLGTQTYICNVNHKPSNTKVDK |
| | | RVEPKSCDKTHTCPPCPAPELLGGPSVFLFPPKPKDTLMISRTPE |
| | | VTCVVVAVSHEDPEVKFNWYVDGVEVHNAKTKPREEQYAST |
| | | YRVVSVLTVLHQDWLNGKEYKCKVSNKALAAPIEKTISKAKG |
| | | QPREPQVYTLPPSREEMTKNQVSLTCLVKGFYPSDIAVEWESN |
| | | GQPENNYKTTPPVLDSDGSFFLYSKLTVDKSRWQQGNVFSCSV |
| | | MHEALHNHYTQKSLSLSPGGGGSGGGGSGGGGSGGGGSEVQL |
| | | VESGGGLVQPGGSLKLSCAASGFTFNTYAMNWVRQASGKCLE |
| | | WVGRIRSKYNNYATYYADSVKDRFTISRDDSKSTLYLQMNSL |
| | | KTEDTAVYYCVRHGNFGNSYVSWFAYWGQGTLVTVSSGGGG |
| | | SGGGGSGGGGSQAVVTQEPSLTVSPGGTVTLTCRSSTGAVTTS |
| | | NYANWVQQKPGQAPRGLIGGTNKRAPWTPARFSGSLLGDKA |
| | | ALTLSGAQPEDEAEYFCALWYSNLWVFGCGTKLTVL |
| 518 | NEG2052 LC | DIQMTQSPSSLSASVGDRVTITCHASQNIYVWLNWYQQKPGK |
| | | APKLLIYKASNLHTGVPSRFSGSGSGTDFTLTISSLQPEDFATYY |
| | | CQQGQTYPYTFGGGTKVEIKRTVAAPSVFIFPPSDEQLKSGTAS |
| | | VVCLLNNFYPREAKVQWKVDNALQSGNSQESVTEQDSKDSTY |
| | | SLSSTLTLSKADYEKHKVYACEVTHQGLSSPVTKSFNRGEC |
| 709 | NEG2052 HC | QVQLVQSGAEVKKPGASVKVSCKASGYTFTSYYIHWVRQAPG |
| | | QGLEWIGCIYPGNVNTNYNEKFKDRATLTVDTSISTAYMELSR |
| | | LRSDDTAVYFCTRSHYGLDWNFDVWGQGTTVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRVEPKSCDKTHTCPPCPAPELLGGPSVFLFPPKPKDTLMISR |
| | | TPEVTCVVVAVSHEDPEVKFNWYVDGVEVHNAKTKPREEQY |
| | | ASTYRVVSVLTVLHQDWLNGKEYKCKVSNKALAAPIEKTISK |
| | | AKGQPREPQVYTLPPSREEMTKNQVSLTCLVKGFYPSDIAVEW |
| | | ESNGQPENNYKTTPPVLDSDGSFFLYSKLTVDKSRWQQGNVFS |
| | | CSVMHEALHNHYTQKSLSLSPGGGGSGGGGSGGGGSGGGGSE |
| | | VQLVESGGGLVQPGGSLKLSCAASGFTFNKYAMNWVRQAPG |
| | | KCLEWVARIRSKYNNYATYYADSVKDRFTISRDDSKNTAYLQ |
| | | MNNLKTEDTAVYYCVRHGNFGNSYISYWAYWGQGTLVTVSS |
| | | GGGGSGGGGSGGGGSGGGGSQTVVTQEPSLTVSPGGTVTLTC |
| | | GSSTGAVTSGNYPNWVQQKPGQAPRGLIGGTKFLAPGTPARFS |
| | | GSLLGGKAALTLSGVQPEDEAEYYCVLWYSNRWVFGCGTKLT |
| | | VL |
| 518 | NEG2053 LC | DIQMTQSPSSLSASVGDRVTITCHASQNIYVWLNWYQQKPGK |
| | | APKLLIYKASNLHTGVPSRFSGSGSGTDFTLTISSLQPEDFATYY |
| | | CQQGQTYPYTFGGGTKVEIKRTVAAPSVFIFPPSDEQLKSGTAS |
| | | VVCLLNNFYPREAKVQWKVDNALQSGNSQESVTEQDSKDSTY |
| | | SLSSTLTLSKADYEKHKVYACEVTHQGLSSPVTKSFNRGEC |
| 710 | NEG2053 HC | QVQLVQSGAEVKKPGASVKVSCKASGYTFTSYYIHWVRQAPG |
| | | QGLEWIGCIYPGNVNTNYNEKFKDRATLTVDTSISTAYMELSR |
| | | LRSDDTAVYFCTRSHYGLDWNFDVWGQGTTVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRVEPKSCDKTHTCPPCPAPELLGGPSVFLFPPKPKDTLMISR |
| | | TPEVTCVVVAVSHEDPEVKFNWYVDGVEVHNAKTKPREEQY |
| | | ASTYRVVSVLTVLHQDWLNGKEYKCKVSNKALAAPIEKTISK |
| | | AKGQPREPQVYTLPPSREEMTKNQVSLTCLVKGFYPSDIAVEW |
| | | ESNGQPENNYKTTPPVLDSDGSFFLYSKLTVDKSRWQQGNVFS |
| | | CSVMHEALHNHYTQKSLSLSPGGGGSGGGGSGGGGSGGGGSE |
| | | VQLVESGGGLVQPGGSLKLSCAASGFTFNTYAMNWVRQASG |
| | | KCLEWVGRIRSKYNNYATYYADSVKDRFTISRDDSKSTLYLQ |
| | | MNSLKTEDTAVYYCVRHGNFGNSYVSWFAYWGQGTLVTVSS |
| | | GGGGSGGGGSGGGGSQAVVTQEPSLTVSPGGTVTLTCRSSTGA |
| | | VTTSNYANWVQQKPGQAPRGLIGGTNKRAPWTPARFSGSLLG |
| | | DKAALTLSGAQPEDEAEYFCALWYSNLWVFGCGTKLTVL |
| 673 | Construct 1 | DVVMTQSPPSLLVTLGQPASISCRSSQSLLHSSGNTYLNWLLQR |
| | Light Chain | PGQSPQPLIYLVSKLESGVPDRFSGSGSGTDFTLKISGVEAEDV |
| | | GVYYCMQFTHYPYTFGQGTKLEIKRTVAAPSVFIFPPSDEQLKS |
| | | GTASVVCLLNNFYPREAKVQWKVDNALQSGNSQESVTEQDSK |
| | | DSTYSLSSTLTLSKADYEKHKVYACEVTHQGLSSPVTKSFNRG |
| | | EC |
| 711 | Construct 1 | QVQLVQSGGGVVQPGRSLRLSCKASGYTFTRYTMHWVRQAP |
| | Heavy Chain | GKGLEWIGYINPSRGYTNYNQKVKDRFTISRDNSKNTAFLQMD |
| | | SLRPEDTGVYFCARYYDDHYSLDYWGQGTPVTVSSGGGGSGG |
| | | GGSGGGGSGGGGSDIQMTQSPSSLSASVGDRVTITCSASSSVSY |
| | | MNWYQQTPGKAPKRWIYDTSKLASGVPSRFSGSGSGTDYTFTI |
| | | SSLQPEDIATYYCQQWSSNPFTFGQGTKLQITGGGGSGGGGSG |
| | | GGGSGGGGSQVQLVQSGAEVQRPGASVKVSCKASGYIFTEYY |
| | | MYWVRQAPGQGLELVGRIDPEDGSIDYVEKFKKKVTLTADTS |
| | | SSTAYMELSSLTSDDTAVYYCARGKFNYRFAYWGQGTLVTVS |
| | | SASTKGPSVFPLAPSSKSTSGGTAALGCLVKDYFPEPVTVSWNS |
| | | GALTSGVHTFPAVLQSSGLYSLSSVVTVPSSSLGTQTYICNVNH |
| | | KPSNTKVDKRVEPKSCDKTHTCPPCPAPELLGGPSVFLFPPKPK |
| | | DTLMISRTPEVTCVVVDVSHEDPEVKFNWYVDGVEVHNAKTK |
| | | PREEQYNSTYRVVSVLTVLHQDWLNGKEYKCKVSNKALPAPI |
| | | EKTISKAKGQPREPQVYTLPPSREEMTKNQVSLTCLVKGFYPS |
| | | DIAVEWESNGQPENNYKTTPPVLDSDGSFFLYSKLTVDKSRWQ |
| | | QGNVFSCSVMHEALHNHYTQKSLSLSPGK |
| 518 | Construct 2 | DIQMTQSPSSLSASVGDRVTITCHASQNIYVWLNWYQQKPGK |
| | Light Chain | APKLLIYKASNLHTGVPSRFSGSGSGTDFTLTISSLQPEDFATYY |
| | | CQQGQTYPYTFGGGTKVEIKRTVAAPSVFIFPPSDEQLKSGTAS |
| | | VVCLLNNFYPREAKVQWKVDNALQSGNSQESVTEQDSKDSTY |
| | | SLSSTLTLSKADYEKHKVYACEVTHQGLSSPVTKSFNRGEC |
| 712 | Construct 2 | QVQLVQSGGGVVQPGRSLRLSCKASGYTFTRYTMHWVRQAP |
| | Heavy Chain | GKGLEWIGYINPSRGYTNYNQKVKDRFTISRDNSKNTAFLQMD |
| | | SLRPEDTGVYFCARYYDDHYSLDYWGQGTPVTVSSGGGGSGG |
| | | GGSGGGGSGGGGSDIQMTQSPSSLSASVGDRVTITCSASSSVSY |
| | | MNWYQQTPGKAPKRWIYDTSKLASGVPSRFSGSGSGTDYTFTI |
| | | SSLQPEDIATYYCQQWSSNPFTFGQGTKLQITGGGGSGGGGSG |
| | | GGGSGGGGSQVQLVQSGAEVKKPGASVKVSCKASGYTFTSYY |
| | | IHWVRQAPGQGLEWIGCIYPGNVNTNYNEKFKDRATLTVDTSI |
| | | STAYMELSRLRSDDTAVYFCTRSHYGLDWNFDVWGQGTTVT |
| | | VSSASTKGPSVFPLAPSSKSTSGGTAALGCLVKDYFPEPVTVSW |
| | | NSGALTSGVHTFPAVLQSSGLYSLSSVVTVPSSSLGTQTYICNV |
| | | NHKPSNTKVDKRVEPKSCDKTHTCPPCPAPELLGGPSVFLFPPK |
| | | PKDTLMISRTPEVTCVVVDVSHEDPEVKFNWYVDGVEVHNAK |
| | | TKPREEQYNSTYRVVSVLTVLHQDWLNGKEYKCKVSNKALPA |
| | | PIEKTISKAKGQPREPQVYTLPPSREEMTKNQVSLTCLVKGFYP |
| | | SDIAVEWESNGQPENNYKTTPPVLDSDGSFFLYSKLTVDKSRW |
| | | QQGNVFSCSVMHEALHNHYTQKSLSLSPGK |
| 673 | Construct 3 | DVVMTQSPPSLLVTLGQPASISCRSSQSLLHSSGNTYLNWLLQR |
| | Light Chain | PGQSPQPLIYLVSKLESGVPDRFSGSGSGTDFTLKISGVEAEDV |
| | | GVYYCMQFTHYPYTFGQGTKLEIKRTVAAPSVFIFPPSDEQLKS |
| | | GTASVVCLLNNFYPREAKVQWKVDNALQSGNSQESVTEQDSK |
| | | DSTYSLSSTLTLSKADYEKHKVYACEVTHQGLSSPVTKSFNRG |
| | | EC |
| 713 | Construct 3 | QVQLVQSGAEVQRPGASVKVSCKASGYIFTEYYMYWVRQAP |
| | Heavy Chain | GQGLELVGRIDPEDGSIDYVEKFKKKVTLTADTSSSTAYMELSS |
| | | LTSDDTAVYYCARGKFNYRFAYWGQGTLVTVSSASTKGPSVF |
| | | PLAPSSKSTSGGTAALGCLVKDYFPEPVTVSWNSGALTSGVHT |
| | | FPAVLQSSGLYSLSSVVTVPSSSLGTQTYICNVNHKPSNTKVDK |
| | | RVEPKSCDKTHTCPPCPAPELLGGPSVFLFPPKPKDTLMISRTPE |
| | | VTCVVVDVSHEDPEVKFNWYVDGVEVHNAKTKPREEQYNST |
| | | YRVVSVLTVLHQDWLNGKEYKCKVSNKALPAPIEKTISKAKG |
| | | QPREPQVYTLPPSREEMTKNQVSLTCLVKGFYPSDIAVEWESN |
| | | GQPENNYKTTPPVLDSDGSFFLYSKLTVDKSRWQQGNVFSCSV |
| | | MHEALHNHYTQKSLSLSPGGGGSGGGGSGGGGSGGGGSQVQ |
| | | LVQSGGGVVQPGRSLRLSCKASGYTFTRYTMHWVRQAPGKG |
| | | LEWIGYINPSRGYTNYNQKVKDRFTISRDNSKNTAFLQMDSLR |
| | | PEDTGVYFCARYYDDHYSLDYWGQGTPVTVSSGGGGSGGGG |
| | | SGGGGSGGGGSDIQMTQSPSSLSASVGDRVTITCSASSSVSYMN |
| | | WYQQTPGKAPKRWIYDTSKLASGVPSRFSGSGSGTDYTFTISSL |
| | | QPEDIATYYCQQWSSNPFTFGQGTKLQIT |
| 518 | Construct 4 | DIQMTQSPSSLSASVGDRVTITCHASQNIYVWLNWYQQKPGK |
| | Light Chain | APKLLIYKASNLHTGVPSRFSGSGSGTDFTLTISSLQPEDFATYY |
| | | CQQGQTYPYTFGGGTKVEIKRTVAAPSVFIFPPSDEQLKSGTAS |
| | | VVCLLNNFYPREAKVQWKVDNALQSGNSQESVTEQDSKDSTY |
| | | SLSSTLTLSKADYEKHKVYACEVTHQGLSSPVTKSFNRGEC |
| 714 | Construct 4 | QVQLVQSGAEVKKPGASVKVSCKASGYTFTSYYIHWVRQAPG |
| | Heavy Chain | QGLEWIGCIYPGNVNTNYNEKFKDRATLTVDTSISTAYMELSR |
| | | LRSDDTAVYFCTRSHYGLDWNFDVWGQGTTVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRVEPKSCDKTHTCPPCPAPELLGGPSVFLFPPKPKDTLMISR |
| | | TPEVTCVVVDVSHEDPEVKFNWYVDGVEVHNAKTKPREEQY |
| | | NSTYRVVSVLTVLHQDWLNGKEYKCKVSNKALPAPIEKTISKA |
| | | KGQPREPQVYTLPPSREEMTKNQVSLTCLVKGFYPSDIAVEWE |
| | | SNGQPENNYKTTPPVLDSDGSFFLYSKLTVDKSRWQQGNVFSC |
| | | SVMHEALHNHYTQKSLSLSPGGGGSGGGGSGGGGSGGGGSQV |
| | | QLVQSGGGVVQPGRSLRLSCKASGYTFTRYTMHWVRQAPGK |
| | | GLEWIGYINPSRGYTNYNQKVKDRFTISRDNSKNTAFLQMDSL |
| | | RPEDTGVYFCARYYDDHYSLDYWGQGTPVTVSSGGGGSGGG |
| | | GSGGGGSGGGGSDIQMTQSPSSLSASVGDRVTITCSASSSVSYM |
| | | NWYQQTPGKAPKRWIYDTSKLASGVPSRFSGSGSGTDYTFTIS |
| | | SLQPEDIATYYCQQWSSNPFTFGQGTKLQIT |
| 673 | Construct 5 | DVVMTQSPPSLLVTLGQPASISCRSSQSLLHSSGNTYLNWLLQR |
| | Light Chain | PGQSPQPLIYLVSKLESGVPDRFSGSGSGTDFTLKISGVEAEDV |
| | | GVYYCMQFTHYPYTFGQGTKLEIKRTVAAPSVFIFPPSDEQLKS |
| | | GTASVVCLLNNFYPREAKVQWKVDNALQSGNSQESVTEQDSK |
| | | DSTYSLSSTLTLSKADYEKHKVYACEVTHQGLSSPVTKSFNRG |
| | | EC |
| 715 | Construct 5 | QVQLVQSGAEVQRPGASVKVSCKASGYIFTEYYMYWVRQAP |
| | Heavy Chain | GQGLELVGRIDPEDGSIDYVEKFKKKVTLTADTSSSTAYMELSS |
| | | LTSDDTAVYYCARGKFNYRFAYWGQGTLVTVSSASTKGPSVF |
| | | PLAPSSKSTSGGTAALGCLVKDYFPEPVTVSWNSGALTSGVHT |
| | | FPAVLQSSGLYSLSSVVTVPSSSLGTQTYICNVNHKPSNTKVDK |
| | | RVEPKSCGGGGSGGGGSQVQLVQSGGGVVQPGRSLRLSCKAS |
| | | GYTFTRYTMHWVRQAPGKGLEWIGYINPSRGYTNYNQKVKD |
| | | RFTISRDNSKNTAFLQMDSLRPEDTGVYFCARYYDDHYSLDY |
| | | WGQGTPVTVSSGGGGSGGGGSGGGGSGGGGSDIQMTQSPSSL |
| | | SASVGDRVTITCSASSSVSYMNWYQQTPGKAPKRWIYDTSKL |
| | | ASGVPSRFSGSGSGTDYTFTISSLQPEDIATYYCQQWSSNPFTFG |
| | | QGTKLQITGGGGSGGGGSGGGGSGGGGSDKTHTCPPCPAPELL |
| | | GGPSVFLFPPKPKDTLMISRTPEVTCVVVDVSHEDPEVKFNWY |
| | | VDGVEVHNAKTKPREEQYNSTYRVVSVLTVLHQDWLNGKEY |
| | | KCKVSNKALPAPIEKTISKAKGQPREPQVYTLPPSREEMTKNQ |
| | | VSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 518 | Construct 6 | DIQMTQSPSSLSASVGDRVTITCHASQNIYVWLNWYQQKPGK |
| | Light Chain | APKLLIYKASNLHTGVPSRFSGSGSGTDFTLTISSLQPEDFATYY |
| | | CQQGQTYPYTFGGGTKVEIKRTVAAPSVFIFPPSDEQLKSGTAS |
| | | VVCLLNNFYPREAKVQWKVDNALQSGNSQESVTEQDSKDSTY |
| | | SLSSTLTLSKADYEKHKVYACEVTHQGLSSPVTKSFNRGEC |
| 716 | Construct 6 | QVQLVQSGAEVKKPGASVKVSCKASGYTFTSYYIHWVRQAPG |
| | Heavy Chain | QGLEWIGCIYPGNVNTNYNEKFKDRATLTVDTSISTAYMELSR |
| | | LRSDDTAVYFCTRSHYGLDWNFDVWGQGTTVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRVEPKSCGGGGSGGGGSQVQLVQSGGGVVQPGRSLRLSCK |
| | | ASGYTFTRYTMHWVRQAPGKGLEWIGYINPSRGYTNYNQKV |
| | | KDRFTISRDNSKNTAFLQMDSLRPEDTGVYFCARYYDDHYSLD |
| | | YWGQGTPVTVSSGGGGSGGGGSGGGGSGGGGSDIQMTQSPSS |
| | | LSASVGDRVTITCSASSSVSYMNWYQQTPGKAPKRWIYDTSKL |
| | | ASGVPSRFSGSGSGTDYTFTISSLQPEDIATYYCQQWSSNPFTFG |
| | | QGTKLQITGGGGSGGGGSGGGGSGGGGSDKTHTCPPCPAPELL |
| | | GGPSVFLFPPKPKDTLMISRTPEVTCVVVDVSHEDPEVKFNWY |
| | | VDGVEVHNAKTKPREEQYNSTYRVVSVLTVLHQDWLNGKEY |
| | | KCKVSNKALPAPIEKTISKAKGQPREPQVYTLPPSREEMTKNQ |
| | | VSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 673 | Construct 7 | DVVMTQSPPSLLVTLGQPASISCRSSQSLLHSSGNTYLNWLLQR |
| | Light Chain | PGQSPQPLIYLVSKLESGVPDRFSGSGSGTDFTLKISGVEAEDV |
| | | GVYYCMQFTHYPYTFGQGTKLEIKRTVAAPSVFIFPPSDEQLKS |
| | | GTASVVCLLNNFYPREAKVQWKVDNALQSGNSQESVTEQDSK |
| | | DSTYSLSSTLTLSKADYEKHKVYACEVTHQGLSSPVTKSFNRG |
| | | EC |
| 717 | Construct 7 | QVQLVQSGGGVVQPGRSLRLSCKASGYTFTRYTMHWVRQAP |
| | Heavy Chain | GKGLEWIGYINPSRGYTNYNQKVKDRFTISRDNSKNTAFLQMD |
| | | SLRPEDTGVYFCARYYDDHYSLDYWGQGTPVTVSSGGGGSGG |
| | | GGSGGGGSGGGGSDIQMTQSPSSLSASVGDRVTITCSASSSVSY |
| | | MNWYQQTPGKAPKRWIYDTSKLASGVPSRFSGSGSGTDYTFTI |
| | | SSLQPEDIATYYCQQWSSNPFTFGQGTKLQITGGGGSDKTHTCP |
| | | PCPAPELLGGPSVFLFPPKPKDTLMISRTPEVTCVVVDVSHEDP |
| | | EVKFNWYVDGVEVHNAKTKPREEQYNSTYRVVSVLTVLHQD |
| | | WLNGKEYKCKVSNKALPAPIEKTISKAKGQPREPQVYTLPPSR |
| | | EEMTKNQVSLTCLVKGFYPSDIAVEWESNGQPENNYKTTPPVL |
| | | DSDGSFFLYSKLTVDKSRWQQGNVFSCSVMHEALHNHYTQKS |
| | | LSLSPGGGGSGGGGSGGGGSGGGGSQVQLVQSGAEVQRPGAS |
| | | VKVSCKASGYIFTEYYMYWVRQAPGQGLELVGRIDPEDGSIDY |
| | | VEKFKKKVTLTADTSSSTAYMELSSLTSDDTAVYYCARGKFN |
| | | YRFAYWGQGTLVTVSSASTKGPSVFPLAPSSKSTSGGTAALGC |
| | | LVKDYFPEPVTVSWNSGALTSGVHTFPAVLQSSGLYSLSSVVT |
| | | VPSSSLGTQTYICNVNHKPSNTKVDKRVEPKSC |
| 518 | Construct 8 | DIQMTQSPSSLSASVGDRVTITCHASQNIYVWLNWYQQKPGK |
| | Light Chain | APKLLIYKASNLHTGVPSRFSGSGSGTDFTLTISSLQPEDFATYY |
| | | CQQGQTYPYTFGGGTKVEIKRTVAAPSVFIFPPSDEQLKSGTAS |
| | | VVCLLNNFYPREAKVQWKVDNALQSGNSQESVTEQDSKDSTY |
| | | SLSSTLTLSKADYEKHKVYACEVTHQGLSSPVTKSFNRGEC |
| 718 | Construct 8 | QVQLVQSGGGVVQPGRSLRLSCKASGYTFTRYTMHWVRQAP |
| | Heavy Chain | GKGLEWIGYINPSRGYTNYNQKVKDRFTISRDNSKNTAFLQMD |
| | | SLRPEDTGVYFCARYYDDHYSLDYWGQGTPVTVSSGGGGSGG |
| | | GGSGGGGSGGGGSDIQMTQSPSSLSASVGDRVTITCSASSSVSY |
| | | MNWYQQTPGKAPKRWIYDTSKLASGVPSRFSGSGSGTDYTFTI |
| | | SSLQPEDIATYYCQQWSSNPFTFGQGTKLQITGGGGSDKTHTCP |
| | | PCPAPELLGGPSVFLFPPKPKDTLMISRTPEVTCVVVDVSHEDP |
| | | EVKFNWYVDGVEVHNAKTKPREEQYNSTYRVVSVLTVLHQD |
| | | WLNGKEYKCKVSNKALPAPIEKTISKAKGQPREPQVYTLPPSR |
| | | EEMTKNQVSLTCLVKGFYPSDIAVEWESNGQPENNYKTTPPVL |
| | | DSDGSFFLYSKLTVDKSRWQQGNVFSCSVMHEALHNHYTQKS |
| | | LSLSPGGGGSGGGGSGGGGSGGGGSQVQLVQSGAEVKKPGAS |
| | | VKVSCKASGYTFTSYYIHWVRQAPGQGLEWIGCIYPGNVNTN |
| | | YNEKFKDRATLTVDTSISTAYMELSRLRSDDTAVYFCTRSHYG |
| | | LDWNFDVWGQGTTVTVSSASTKGPSVFPLAPSSKSTSGGTAAL |
| | | GCLVKDYFPEPVTVSWNSGALTSGVHTFPAVLQSSGLYSLSSV |
| | | VTVPSSSLGTQTYICNVNHKPSNTKVDKRVEPKSC |
| 673 | Construct 9 | DVVMTQSPPSLLVTLGQPASISCRSSQSLLHSSGNTYLNWLLQR |
| | Light Chain | PGQSPQPLIYLVSKLESGVPDRFSGSGSGTDFTLKISGVEAEDV |
| | | GVYYCMQFTHYPYTFGQGTKLEIKRTVAAPSVFIFPPSDEQLKS |
| | | GTASVVCLLNNFYPREAKVQWKVDNALQSGNSQESVTEQDSK |
| | | DSTYSLSSTLTLSKADYEKHKVYACEVTHQGLSSPVTKSFNRG |
| | | EC |
| 719 | Construct 9 | QVQLVQSGAEVQRPGASVKVSCKASGYIFTEYYMYWVRQAP |
| | Heavy Chain 1 | GQGLELVGRIDPEDGSIDYVEKFKKKVTLTADTSSSTAYMELSS |
| | | LTSDDTAVYYCARGKFNYRFAYWGQGTLVTVSSASTKGPSVF |
| | | PLAPSSKSTSGGTAALGCLVKDYFPEPVTVSWNSGALTSGVHT |
| | | FPAVLQSSGLYSLSSVVTVPSSSLGTQTYICNVNHKPSNTKVDK |
| | | RVEPKSCDKTHTCPPCPAPELLGGPSVFLFPPKPKDTLMISRTPE |
| | | VTCVVVDVSHEDPEVKFNWYVDGVEVHNAKTKPREEQYNST |
| | | YRVVSVLTVLHQDWLNGKEYKCKVSNKALPAPIEKTISKAKG |
| | | QPREPQVCTLPPSREEMTKNQVSLSCAVKGFYPSDIAVEWESN |
| | | GQPENNYKTTPPVLDSDGSFFLVSKLTVDKSRWQQGNVFSCSV |
| | | MHEALHNRFTQKSLSLSPGK |
| 720 | Construct 9 | QVQLVQSGGGVVQPGRSLRLSCKASGYTFTRYTMHWVRQAP |
| | Heavy Chain 2 | GKGLEWIGYINPSRGYTNYNQKVKDRFTISRDNSKNTAFLQMD |
| | | SLRPEDTGVYFCARYYDDHYSLDYWGQGTPVTVSSGGGGSGG |
| | | GGSGGGGSGGGGSDIQMTQSPSSLSASVGDRVTITCSASSSVSY |
| | | MNWYQQTPGKAPKRWIYDTSKLASGVPSRFSGSGSGTDYTFTI |
| | | SSLQPEDIATYYCQQWSSNPFTFGQGTKLQITGGGGSDKTHTCP |
| | | PCPAPELLGGPSVFLFPPKPKDTLMISRTPEVTCVVVDVSHEDP |
| | | EVKFNWYVDGVEVHNAKTKPREEQYNSTYRVVSVLTVLHQD |
| | | WLNGKEYKCKVSNKALPAPIEKTISKAKGQPREPQVYTLPPCR |
| | | EEMTKNQVSLWCLVKGFYPSDIAVEWESNGQPENNYKTTPPV |
| | | LDSDGSFFLYSKLTVDKSRWQQGNVFSCSVMHEALHNHYTQK |
| | | SLSLSPGK |
| 518 | Construct 10 | DIQMTQSPSSLSASVGDRVTITCHASQNIYVWLNWYQQKPGK |
| | Light Chain | APKLLIYKASNLHTGVPSRFSGSGSGTDFTLTISSLQPEDFATYY |
| | | CQQGQTYPYTFGGGTKVEIKRTVAAPSVFIFPPSDEQLKSGTAS |
| | | VVCLLNNFYPREAKVQWKVDNALQSGNSQESVTEQDSKDSTY |
| | | SLSSTLTLSKADYEKHKVYACEVTHQGLSSPVTKSFNRGEC |
| 721 | Construct 10 | QVQLVQSGAEVKKPGASVKVSCKASGYTFTSYYIHWVRQAPG |
| | Heavy Chain 1 | QGLEWIGCIYPGNVNTNYNEKFKDRATLTVDTSISTAYMELSR |
| | | LRSDDTAVYFCTRSHYGLDWNFDVWGQGTTVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRVEPKSCDKTHTCPPCPAPELLGGPSVFLFPPKPKDTLMISR |
| | | TPEVTCVVVDVSHEDPEVKFNWYVDGVEVHNAKTKPREEQY |
| | | NSTYRVVSVLTVLHQDWLNGKEYKCKVSNKALPAPIEKTISKA |
| | | KGQPREPQVCTLPPSREEMTKNQVSLSCAVKGFYPSDIAVEWE |
| | | SNGQPENNYKTTPPVLDSDGSFFLVSKLTVDKSRWQQGNVFSC |
| | | SVMHEALHNRFTQKSLSLSPGK |
| 720 | Construct 10 | QVQLVQSGGGVVQPGRSLRLSCKASGYTFTRYTMHWVRQAP |
| | Heavy Chain 2 | GKGLEWIGYINPSRGYTNYNQKVKDRFTISRDNSKNTAFLQMD |
| | | SLRPEDTGVYFCARYYDDHYSLDYWGQGTPVTVSSGGGGSGG |
| | | GGSGGGGSGGGGSDIQMTQSPSSLSASVGDRVTITCSASSSVSY |
| | | MNWYQQTPGKAPKRWIYDTSKLASGVPSRFSGSGSGTDYTFTI |
| | | SSLQPEDIATYYCQQWSSNPFTFGQGTKLQITGGGGSDKTHTCP |
| | | PCPAPELLGGPSVFLFPPKPKDTLMISRTPEVTCVVVDVSHEDP |
| | | EVKFNWYVDGVEVHNAKTKPREEQYNSTYRVVSVLTVLHQD |
| | | WLNGKEYKCKVSNKALPAPIEKTISKAKGQPREPQVYTLPPCR |
| | | EEMTKNQVSLWCLVKGFYPSDIAVEWESNGQPENNYKTTPPV |
| | | LDSDGSFFLYSKLTVDKSRWQQGNVFSCSVMHEALHNHYTQK |
| | | SLSLSPGK |
| 722 | Construct 11 | DKTHTCPPCPAPELLGGPSVFLFPPKPKDTLMISRTPEVTCVVV |
| | Heavy Chain 1 | DVSHEDPEVKFNWYVDGVEVHNAKTKPREEQYNSTYRVVSV |
| | | LTVLHQDWLNGKEYKCKVSNKALPAPIEKTISKAKGQPREPQV |
| | | CTLPPSREEMTKNQVSLSCAVKGFYPSDIAVEWESNGQPENNY |
| | | KTTPPVLDSDGSFFLVSKLTVDKSRWQQGNVFSCSVMHEALH |
| | | NRFTQKSLSLSPGK |
| 720 | Construct 11 | QVQLVQSGGGVVQPGRSLRLSCKASGYTFTRYTMHWVRQAP |
| | Heavy Chain 2 | GKGLEWIGYINPSRGYTNYNQKVKDRFTISRDNSKNTAFLQMD |
| | | SLRPEDTGVYFCARYYDDHYSLDYWGQGTPVTVSSGGGGSGG |
| | | GGSGGGGSGGGGSDIQMTQSPSSLSASVGDRVTITCSASSSVSY |
| | | MNWYQQTPGKAPKRWIYDTSKLASGVPSRFSGSGSGTDYTFTI |
| | | SSLQPEDIATYYCQQWSSNPFTFGQGTKLQITGGGGSDKTHTCP |
| | | PCPAPELLGGPSVFLFPPKPKDTLMISRTPEVTCVVVDVSHEDP |
| | | EVKFNWYVDGVEVHNAKTKPREEQYNSTYRVVSVLTVLHQD |
| | | WLNGKEYKCKVSNKALPAPIEKTISKAKGQPREPQVYTLPPCR |
| | | EEMTKNQVSLWCLVKGFYPSDIAVEWESNGQPENNYKTTPPV |
| | | LDSDGSFFLYSKLTVDKSRWQQGNVFSCSVMHEALHNHYTQK |
| | | SLSLSPGK |
| 673 | Construct 12 | DVVMTQSPPSLLVTLGQPASISCRSSQSLLHSSGNTYLNWLLQR |
| | Light Chain | PGQSPQPLIYLVSKLESGVPDRFSGSGSGTDFTLKISGVEAEDV |
| | | GVYYCMQFTHYPYTFGQGTKLEIKRTVAAPSVFIFPPSDEQLKS |
| | | GTASVVCLLNNFYPREAKVQWKVDNALQSGNSQESVTEQDSK |
| | | DSTYSLSSTLTLSKADYEKHKVYACEVTHQGLSSPVTKSFNRG |
| | | EC |
| 719 | Construct 12 | QVQLVQSGAEVQRPGASVKVSCKASGYIFTEYYMYWVRQAP |
| | Heavy Chain 1 | GQGLELVGRIDPEDGSIDYVEKFKKKVTLTADTSSSTAYMELSS |
| | | LTSDDTAVYYCARGKFNYRFAYWGQGTLVTVSSASTKGPSVF |
| | | PLAPSSKSTSGGTAALGCLVKDYFPEPVTVSWNSGALTSGVHT |
| | | FPAVLQSSGLYSLSSVVTVPSSSLGTQTYICNVNHKPSNTKVDK |
| | | RVEPKSCDKTHTCPPCPAPELLGGPSVFLFPPKPKDTLMISRTPE |
| | | VTCVVVDVSHEDPEVKFNWYVDGVEVHNAKTKPREEQYNST |
| | | YRVVSVLTVLHQDWLNGKEYKCKVSNKALPAPIEKTISKAKG |
| | | QPREPQVCTLPPSREEMTKNQVSLSCAVKGFYPSDIAVEWESN |
| | | GQPENNYKTTPPVLDSDGSFFLVSKLTVDKSRWQQGNVFSCSV |
| | | MHEALHNRFTQKSLSLSPGK |
| 723 | Construct 12 | QVQLVQSGGGVVQPGRSLRLSCKASGYTFTRYTMHWVRQAP |
| | Heavy Chain 2 | GKGLEWIGYINPSRGYTNYNQKVKDRFTISRDNSKNTAFLQMD |
| | | SLRPEDTGVYFCARYYDDHYSLDYWGQGTPVTVSSGGGGSGG |
| | | GGSGGGGSGGGGSDIQMTQSPSSLSASVGDRVTITCSASSSVSY |
| | | MNWYQQTPGKAPKRWIYDTSKLASGVPSRFSGSGSGTDYTFTI |
| | | SSLQPEDIATYYCQQWSSNPFTFGQGTKLQITGGGGSDKTHTCP |
| | | PCPAPELLGGPSVFLFPPKPKDTLMISRTPEVTCVVVDVSHEDP |
| | | EVKFNWYVDGVEVHNAKTKPREEQYNSTYRVVSVLTVLHQD |
| | | WLNGKEYKCKVSNKALPAPIEKTISKAKGQPREPQVYTLPPCR |
| | | EEMTKNQVSLWCLVKGFYPSDIAVEWESNGQPENNYKTTPPV |
| | | LDSDGSFFLYSKLTVDKSRWQQGNVFSCSVMHEALHNHYTQK |
| | | SLSLSPGGGGGSGGGGSGGGGSEEDPCACESLVKFQAKVEGLL |
| | | QALTRKLEAVSKRLAILENTVV |
| 518 | Construct 13 | DIQMTQSPSSLSASVGDRVTITCHASQNIYVWLNWYQQKPGK |
| | Light Chain | APKLLIYKASNLHTGVPSRFSGSGSGTDFTLTISSLQPEDFATYY |
| | | CQQGQTYPYTFGGGTKVEIKRTVAAPSVFIFPPSDEQLKSGTAS |
| | | VVCLLNNFYPREAKVQWKVDNALQSGNSQESVTEQDSKDSTY |
| | | SLSSTLTLSKADYEKHKVYACEVTHQGLSSPVTKSFNRGEC |
| 721 | Construct 13 | QVQLVQSGAEVKKPGASVKVSCKASGYTFTSYYIHWVRQAPG |
| | Heavy Chain 1 | QGLEWIGCIYPGNVNTNYNEKFKDRATLTVDTSISTAYMELSR |
| | | LRSDDTAVYFCTRSHYGLDWNFDVWGQGTTVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRVEPKSCDKTHTCPPCPAPELLGGPSVFLFPPKPKDTLMISR |
| | | TPEVTCVVVDVSHEDPEVKFNWYVDGVEVHNAKTKPREEQY |
| | | NSTYRVVSVLTVLHQDWLNGKEYKCKVSNKALPAPIEKTISKA |
| | | KGQPREPQVCTLPPSREEMTKNQVSLSCAVKGFYPSDIAVEWE |
| | | SNGQPENNYKTTPPVLDSDGSFFLVSKLTVDKSRWQQGNVFSC |
| | | SVMHEALHNRFTQKSLSLSPGK |
| 723 | Construct 13 | QVQLVQSGGGVVQPGRSLRLSCKASGYTFTRYTMHWVRQAP |
| | Heavy Chain 2 | GKGLEWIGYINPSRGYTNYNQKVKDRFTISRDNSKNTAFLQMD |
| | | SLRPEDTGVYFCARYYDDHYSLDYWGQGTPVTVSSGGGGSGG |
| | | GGSGGGGSGGGGSDIQMTQSPSSLSASVGDRVTITCSASSSVSY |
| | | MNWYQQTPGKAPKRWIYDTSKLASGVPSRFSGSGSGTDYTFTI |
| | | SSLQPEDIATYYCQQWSSNPFTFGQGTKLQITGGGGSDKTHTCP |
| | | PCPAPELLGGPSVFLFPPKPKDTLMISRTPEVTCVVVDVSHEDP |
| | | EVKFNWYVDGVEVHNAKTKPREEQYNSTYRVVSVLTVLHQD |
| | | WLNGKEYKCKVSNKALPAPIEKTISKAKGQPREPQVYTLPPCR |
| | | EEMTKNQVSLWCLVKGFYPSDIAVEWESNGQPENNYKTTPPV |
| | | LDSDGSFFLYSKLTVDKSRWQQGNVFSCSVMHEALHNHYTQK |
| | | SLSLSPGGGGGSGGGGSGGGGSEEDPCACESLVKFQAKVEGLL |
| | | QALTRKLEAVSKRLAILENTVV |
| 722 | Construct 14 | DKTHTCPPCPAPELLGGPSVFLFPPKPKDTLMISRTPEVTCVVV |
| | Heavy Chain 1 | DVSHEDPEVKFNWYVDGVEVHNAKTKPREEQYNSTYRVVSV |
| | | LTVLHQDWLNGKEYKCKVSNKALPAPIEKTISKAKGQPREPQV |
| | | CTLPPSREEMTKNQVSLSCAVKGFYPSDIAVEWESNGQPENNY |
| | | KTTPPVLDSDGSFFLVSKLTVDKSRWQQGNVFSCSVMHEALH |
| | | NRFTQKSLSLSPGK |
| 723 | Construct 14 | QVQLVQSGGGVVQPGRSLRLSCKASGYTFTRYTMHWVRQAP |
| | Heavy Chain 2 | GKGLEWIGYINPSRGYTNYNQKVKDRFTISRDNSKNTAFLQMD |
| | | SLRPEDTGVYFCARYYDDHYSLDYWGQGTPVTVSSGGGGSGG |
| | | GGSGGGGSGGGGSDIQMTQSPSSLSASVGDRVTITCSASSSVSY |
| | | MNWYQQTPGKAPKRWIYDTSKLASGVPSRFSGSGSGTDYTFTI |
| | | SSLQPEDIATYYCQQWSSNPFTFGQGTKLQITGGGGSDKTHTCP |
| | | PCPAPELLGGPSVFLFPPKPKDTLMISRTPEVTCVVVDVSHEDP |
| | | EVKFNWYVDGVEVHNAKTKPREEQYNSTYRVVSVLTVLHQD |
| | | WLNGKEYKCKVSNKALPAPIEKTISKAKGQPREPQVYTLPPCR |
| | | EEMTKNQVSLWCLVKGFYPSDIAVEWESNGQPENNYKTTPPV |
| | | LDSDGSFFLYSKLTVDKSRWQQGNVFSCSVMHEALHNHYTQK |
| | | SLSLSPGGGGGSGGGGSGGGGSEEDPCACESLVKFQAKVEGLL |
| | | QALTRKLEAVSKRLAILENTVV |
| 673 | Construct 15 | DVVMTQSPPSLLVTLGQPASISCRSSQSLLHSSGNTYLNWLLQR |
| | Light Chain | PGQSPQPLIYLVSKLESGVPDRFSGSGSGTDFTLKISGVEAEDV |
| | | GVYYCMQFTHYPYTFGQGTKLEIKRTVAAPSVFIFPPSDEQLKS |
| | | GTASVVCLLNNFYPREAKVQWKVDNALQSGNSQESVTEQDSK |
| | | DSTYSLSSTLTLSKADYEKHKVYACEVTHQGLSSPVTKSFNRG |
| | | EC |
| 719 | Construct 15 | QVQLVQSGAEVQRPGASVKVSCKASGYIFTEYYMYWVRQAP |
| | Heavy Chain 1 | GQGLELVGRIDPEDGSIDYVEKFKKKVTLTADTSSSTAYMELSS |
| | | LTSDDTAVYYCARGKFNYRFAYWGQGTLVTVSSASTKGPSVF |
| | | PLAPSSKSTSGGTAALGCLVKDYFPEPVTVSWNSGALTSGVHT |
| | | FPAVLQSSGLYSLSSVVTVPSSSLGTQTYICNVNHKPSNTKVDK |
| | | RVEPKSCDKTHTCPPCPAPELLGGPSVFLFPPKPKDTLMISRTPE |
| | | VTCVVVDVSHEDPEVKFNWYVDGVEVHNAKTKPREEQYNST |
| | | YRVVSVLTVLHQDWLNGKEYKCKVSNKALPAPIEKTISKAKG |
| | | QPREPQVCTLPPSREEMTKNQVSLSCAVKGFYPSDIAVEWESN |
| | | GQPENNYKTTPPVLDSDGSFFLVSKLTVDKSRWQQGNVFSCSV |
| | | MHEALHNRFTQKSLSLSPGK |
| 724 | Construct 15 | QVQLVQSGGGVVQPGRSLRLSCKASGYTFTRYTMHWVRQAP |
| | Heavy Chain 2 | GKGLEWIGYINPSRGYTNYNQKVKDRFTISRDNSKNTAFLQMD |
| | | SLRPEDTGVYFCARYYDDHYSLDYWGQGTPVTVSSGGGGSGG |
| | | GGSGGGGSGGGGSDIQMTQSPSSLSASVGDRVTITCSASSSVSY |
| | | MNWYQQTPGKAPKRWIYDTSKLASGVPSRFSGSGSGTDYTFTI |
| | | SSLQPEDIATYYCQQWSSNPFTFGQGTKLQITGGGGSDKTHTCP |
| | | PCPAPELLGGPSVFLFPPKPKDTLMISRTPEVTCVVVDVSHEDP |
| | | EVKFNWYVDGVEVHNAKTKPREEQYNSTYRVVSVLTVLHQD |
| | | WLNGKEYKCKVSNKALPAPIEKTISKAKGQPREPQVYTLPPCR |
| | | EEMTKNQVSLWCLVKGFYPSDIAVEWESNGQPENNYKTTPPV |
| | | LDSDGSFFLYSKLTVDKSRWQQGNVFSCSVMHEALHNHYTQK |
| | | SLSLSPGGGGGSDLGPQMLRELQETNAALQDVRELLRQQVREI |
| | | TFLKNTVMECDACGMQQ |
| 518 | Construct 16 | DIQMTQSPSSLSASVGDRVTITCHASQNIYVWLNWYQQKPGK |
| | Light Chain | APKLLIYKASNLHTGVPSRFSGSGSGTDFTLTISSLQPEDFATYY |
| | | CQQGQTYPYTFGGGTKVEIKRTVAAPSVFIFPPSDEQLKSGTAS |
| | | VVCLLNNFYPREAKVQWKVDNALQSGNSQESVTEQDSKDSTY |
| | | SLSSTLTLSKADYEKHKVYACEVTHQGLSSPVTKSFNRGEC |
| 721 | Construct 16 | QVQLVQSGAEVKKPGASVKVSCKASGYTFTSYYIHWVRQAPG |
| | Heavy Chain 1 | QGLEWIGCIYPGNVNTNYNEKFKDRATLTVDTSISTAYMELSR |
| | | LRSDDTAVYFCTRSHYGLDWNFDVWGQGTTVTVSSASTKGPS |
| | | VFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRVEPKSCDKTHTCPPCPAPELLGGPSVFLFPPKPKDTLMISR |
| | | TPEVTCVVVDVSHEDPEVKFNWYVDGVEVHNAKTKPREEQY |
| | | NSTYRVVSVLTVLHQDWLNGKEYKCKVSNKALPAPIEKTISKA |
| | | KGQPREPQVCTLPPSREEMTKNQVSLSCAVKGFYPSDIAVEWE |
| | | SNGQPENNYKTTPPVLDSDGSFFLVSKLTVDKSRWQQGNVFSC |
| | | SVMHEALHNRFTQKSLSLSPGK |
| 724 | Construct 16 | QVQLVQSGGGVVQPGRSLRLSCKASGYTFTRYTMHWVRQAP |
| | Heavy Chain 2 | GKGLEWIGYINPSRGYTNYNQKVKDRFTISRDNSKNTAFLQMD |
| | | SLRPEDTGVYFCARYYDDHYSLDYWGQGTPVTVSSGGGGSGG |
| | | GGSGGGGSGGGGSDIQMTQSPSSLSASVGDRVTITCSASSSVSY |
| | | MNWYQQTPGKAPKRWIYDTSKLASGVPSRFSGSGSGTDYTFTI |
| | | SSLQPEDIATYYCQQWSSNPFTFGQGTKLQITGGGGSDKTHTCP |
| | | PCPAPELLGGPSVFLFPPKPKDTLMISRTPEVTCVVVDVSHEDP |
| | | EVKFNWYVDGVEVHNAKTKPREEQYNSTYRVVSVLTVLHQD |
| | | WLNGKEYKCKVSNKALPAPIEKTISKAKGQPREPQVYTLPPCR |
| | | EEMTKNQVSLWCLVKGFYPSDIAVEWESNGQPENNYKTTPPV |
| | | LDSDGSFFLYSKLTVDKSRWQQGNVFSCSVMHEALHNHYTQK |
| | | SLSLSPGGGGGSDLGPQMLRELQETNAALQDVRELLRQQVREI |
| | | TFLKNTVMECDACGMQQ |
| 722 | Construct 17 | DKTHTCPPCPAPELLGGPSVFLFPPKPKDTLMISRTPEVTCVVV |
| | Heavy Chain 1 | DVSHEDPEVKFNWYVDGVEVHNAKTKPREEQYNSTYRVVSV |
| | | LTVLHQDWLNGKEYKCKVSNKALPAPIEKTISKAKGQPREPQV |
| | | CTLPPSREEMTKNQVSLSCAVKGFYPSDIAVEWESNGQPENNY |
| | | KTTPPVLDSDGSFFLVSKLTVDKSRWQQGNVFSCSVMHEALH |
| | | NRFTQKSLSLSPGK |
| 724 | Construct 17 | QVQLVQSGGGVVQPGRSLRLSCKASGYTFTRYTMHWVRQAP |
| | Heavy Chain 2 | GKGLEWIGYINPSRGYTNYNQKVKDRFTISRDNSKNTAFLQMD |
| | | SLRPEDTGVYFCARYYDDHYSLDYWGQGTPVTVSSGGGGSGG |
| | | GGSGGGGSGGGGSDIQMTQSPSSLSASVGDRVTITCSASSSVSY |
| | | MNWYQQTPGKAPKRWIYDTSKLASGVPSRFSGSGSGTDYTFTI |
| | | SSLQPEDIATYYCQQWSSNPFTFGQGTKLQITGGGGSDKTHTCP |
| | | PCPAPELLGGPSVFLFPPKPKDTLMISRTPEVTCVVVDVSHEDP |
| | | EVKFNWYVDGVEVHNAKTKPREEQYNSTYRVVSVLTVLHQD |
| | | WLNGKEYKCKVSNKALPAPIEKTISKAKGQPREPQVYTLPPCR |
| | | EEMTKNQVSLWCLVKGFYPSDIAVEWESNGQPENNYKTTPPV |
| | | LDSDGSFFLYSKLTVDKSRWQQGNVFSCSVMHEALHNHYTQK |
| | | SLSLSPGGGGGSDLGPQMLRELQETNAALQDVRELLRQQVREI |
| | | TFLKNTVMECDACGMQQ |
| SEQ ID | Anti-CD3 | QVQLVESGGGLVQPGGSLRLSCAASGFTFSTYGMSWVR |
| NO: 794 | (4)/anti-CD28 | QAPGKGLEWVSSIFYTGSSTYYADSVKGRFTISRDNSKNT |
| | (2) bispecific | LYLQMNSLRAEDTAVYYCARIGYAGDSKYAIWGQGTLV |
| | heavy chain | TVSSASTKGPSVFPLAPSSKSTSGGTAALGCLVKDYFPEP |
| | | VTVSWNSGALTSGVHTFPAVLQSSGLYSLSSVVTVPSSSL |
| | | GTQTYICNVNHKPSNTKVDKRVEPKSCDKTHTCPPCPAP |
| | | EAAGGPSVFLFPPKPKDTLMISRTPEVTCVVVDVKHEDPE |
| | | VKFNWYVDGVEVHNAKTKPREEQYNSTYRVVSVLTVLH |
| | | QDWLNGKEYKCKVSNKALAAPIEKTISKAKGQPREPQVY |
| | | TLPPSREEMTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPGGGGGSGGGGSGGGGSGGGGS |
| | | EVKLVESGGDLVQPGDSLTLSCVASGFTFSKQGMHWIRQ |
| | | APKKGLEWIAMIYYDSSKMYYADTVKGRFTISRDNSKNT |
| | | LYLEMNSLRSEDTAMYYCASFWWDLDFDHWGQGVMVT |
| | | VSSGGGGSGGGGSGGGGSGGGGSDILVTQTPVSLPVSLG |
| | | GHVSISCRSSQSLVRSEGTTYFNWYLQKPGQSPQLLIYRV |
| | | SNRFSGVPDRFSGSGSGTDFTLKISRVEPEDLGVYYCLQSS |
| | | HFPWTFGGGTKLELK |
| SEQ ID | Anti-CD3 | EVQLVESGGGLVQPGGSLRLSCAASGFTFSTYGMSWVRQ |
| NO: 795 | (4)/anti-CD28 | APGKGLEWVSSIFYTGSSTYYADSVKGRFTISRDNSKNTL |
| | (2) bispecific | YLQMNSLRAEDTAVYYCARIGYAGDSKYAIWGQGTLVT |
| | alternative | VSSASTKGPSVFPLAPSSKSTSGGTAALGCLVKDYFPEPV |
| | heavy chain | TVSWNSGALTSGVHTFPAVLQSSGLYSLSSVVTVPSSSLG |
| | | TQTYICNVNHKPSNTKVDKRVEPKSCDKTHTCPPCPAPE |
| | | AAGGPSVFLFPPKPKDTLMISRTPEVTCVVVDVKHEDPEV |
| | | KFNWYVDGVEVHNAKTKPREEQYNSTYRVVSVLTVLHQ |
| | | DWLNGKEYKCKVSNKALAAPIEKTISKAKGQPREPQVYT |
| | | LPPSREEMTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPGGGGGSGGGGSGGGGSGGGGS |
| | | EVKLVESGGDLVQPGDSLTLSCVASGFTFSKQGMHWIRQ |
| | | APKKGLEWIAMIYYDSSKMYYADTVKGRFTISRDNSKNT |
| | | LYLEMNSLRSEDTAMYYCASFWWDLDFDHWGQGVMVT |
| | | VSSGGGGSGGGGSGGGGSGGGGSDILVTQTPVSLPVSLG |
| | | GHVSISCRSSQSLVRSEGTTYFNWYLQKPGQSPQLLIYRV |
| | | SNRFSGVPDRFSGSGSGTDFTLKISRVEPEDLGVYYCLQSS |
| | | HFPWTFGGGTKLELK |
| SEQ ID | Anti-CD3 | DIVLTQPPSVSGAPGQRVTISCSGSSSNIVSNYVNWYQQL |
| NO: 796 | (4)/anti-CD28 | PGTAPKLLIYDNNKRPSGVPDRFSGSKSGTSASLAITGLQS |
| | (2) bispecific | EDEADYYCQSYAIGSYSVVFGGGTKLTVLGQGTKVEIKR |
| | light chain 1 | TVAAPSVFIFPPSDEQLKSGTASVVCLLNNFYPREAKVQW |
| | | KVDNALQSGNSQESVTEQDSKDSTYSLSSTLTLSKADYE |
| | | KHKVYACEVTHQGLSSPVTKSFNRGEC |
| SEQ ID | Anti-CD3 | DIVLTQPPSVSGAPGQRVTISCSGSSSNIVSNYVNWYQQL |
| NO: 797 | (4)/anti-CD28 | PGTAPKLLIYDNNKRPSGVPDRFSGSKSGTSASLAITGLQS |
| | (2) bispecific | EDEADYYCQSYAIGSYSVVFGGGTKLTVLGQPKAAPSVT |
| | light chain 2 | LFPPSSEELQANKATLVCLISDFYPGAVTVAWKADSSPVK |
| | | AGVETTTPSKQSNNKYAASSYLSLTPEQWKSHRSYSCQV |
| | | THEGSTVEKTVAPTECS |
| SEQ ID | Anti-CD3 | QVQLVESGGGLVQPGGSLRLSCAASGFTFSTYGMSWVR |
| NO: 798 | (2)/anti-CD28 | QAPGKGLEWVSSIFYTGSSTYYADSVKGRFTISRDNSKNT |
| | (2) heavy | LYLQMNSLRAEDTAVYYCARIGYAGDSKYAIWGQGTLV |
| | chain | TVSSASTKGPSVFPLAPSSKSTSGGTAALGCLVKDYFPEP |
| | | VTVSWNSGALTSGVHTFPAVLQSSGLYSLSSVVTVPSSSL |
| | | GTQTYICNVNHKPSNTKVDKRVEPKSCDKTHTCPPCPAP |
| | | EAAGGPSVFLFPPKPKDTLMISRTPEVTCVVVDVKHEDPE |
| | | VKFNWYVDGVEVHNAKTKPREEQYNSTYRVVSVLTVLH |
| | | QDWLNGKEYKCKVSNKALAAPIEKTISKAKGQPREPQVY |
| | | TLPPSREEMTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPGGGGGSGGGGSGGGGSGGGGS |
| | | EVQLVESGGGLVQPGGSLKLSCAASGFTFNKYAMNWVR |
| | | QAPGKCLEWVARIRSKYNNYATYYADSVKDRFTISRDDS |
| | | KNTAYLQMNNLKTEDTAVYYCVRHGNFGNSYISYWAY |
| | | WGQGTLVTVSSGGGGSGGGGSGGGGSGGGGSQTVVTQE |
| | | PSLTVSPGGTVTLTCGSSTGAVTSGNYPNWVQQKPGQAP |
| | | RGLIGGTKFLAPGTPARFSGSLLGGKAALTLSGVQPEDEA |
| | | EYYCVLWYSNRWVFGCGTKLTVL |
| SEQ ID | Anti-CD3 | EVQLVESGGGLVQPGGSLRLSCAASGFTFSTYGMSWVRQ |
| NO: 815 | (2)/anti-CD28 | APGKGLEWVSSIFYTGSSTYYADSVKGRFTISRDNSKNTL |
| | (2) alternative | YLQMNSLRAEDTAVYYCARIGYAGDSKYAIWGQGTLVT |
| | heavy chain | VSSASTKGPSVFPLAPSSKSTSGGTAALGCLVKDYFPEPV |
| | | TVSWNSGALTSGVHTFPAVLQSSGLYSLSSVVTVPSSSLG |
| | | TQTYICNVNHKPSNTKVDKRVEPKSCDKTHTCPPCPAPE |
| | | AAGGPSVFLFPPKPKDTLMISRTPEVTCVVVDVKHEDPEV |
| | | KFNWYVDGVEVHNAKTKPREEQYNSTYRVVSVLTVLHQ |
| | | DWLNGKEYKCKVSNKALAAPIEKTISKAKGQPREPQVYT |
| | | LPPSREEMTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPGGGGGSGGGGSGGGGSGGGGS |
| | | EVQLVESGGGLVQPGGSLKLSCAASGFTFNKYAMNWVR |
| | | QAPGKCLEWVARIRSKYNNYATYYADSVKDRFTISRDDS |
| | | KNTAYLQMNNLKTEDTAVYYCVRHGNFGNSYISYWAY |
| | | WGQGTLVTVSSGGGGSGGGGSGGGGSGGGGSQTVVTQE |
| | | PSLTVSPGGTVTLTCGSSTGAVTSGNYPNWVQQKPGQAP |
| | | RGLIGGTKFLAPGTPARFSGSLLGGKAALTLSGVQPEDEA |
| | | EYYCVLWYSNRWVFGCGTKLTVL |
| SEQ ID | Anti-CD3 | DIVLTQPPSVSGAPGQRVTISCSGSSSNIVSNYVNWYQQL |
| NO: 799 | (2)/anti-CD28 | PGTAPKLLIYDNNKRPSGVPDRFSGSKSGTSASLAITGLQS |
| | (2) light chain | EDEADYYCQSYAIGSYSVVFGGGTKLTVLGQGTKVEIKR |
| | | TVAAPSVFIFPPSDEQLKSGTASVVCLLNNFYPREAKVQW |
| | | KVDNALQSGNSQESVTEQDSKDSTYSLSSTLTLSKADYE |
| | | KHKVYACEVTHQGLSSPVTKSFNRGEC |
| SEQ ID | Anti-CD3 | QVQLVQSGAEVKKPGASVKVSCKASGYTFTSYYIHWVR |
| NO: 800 | (4)/anti-CD28 | QAPGQGLEWIGCIYPGNVNTNYNEKFKDRATLTVDTSIST |
| | (1) heavy | AYMELSRLRSDDTAVYFCTRSHYGLDWNFDVWGQGTT |
| | chain | VTVSSASTKGPSVFPLAPSSKSTSGGTAALGCLVKDYFPE |
| | | PVTVSWNSGALTSGVHTFPAVLQSSGLYSLSSVVTVPSSS |
| | | LGTQTYICNVNHKPSNTKVDKRVEPKSCDKTHTCPPCPA |
| | | PEAAGGPSVFLFPPKPKDTLMISRTPEVTCVVVDVKHEDP |
| | | EVKFNWYVDGVEVHNAKTKPREEQYNSTYRVVSVLTVL |
| | | HQDWLNGKEYKCKVSNKALAAPIEKTISKAKGQPREPQV |
| | | YTLPPSREEMTKNQVSLTCLVKGFYPSDIAVEWESNGQPE |
| | | NNYKTTPPVLDSDGSFFLYSKLTVDKSRWQQGNVFSCSV |
| | | MHEALHNHYTQKSLSLSPGGGSEVKLVESGGDLVQPGDS |
| | | LTLSCVASGFTFSKQGMHWIRQAPKKGLEWIAMIYYDSS |
| | | KMYYADTVKGRFTISRDNSKNTLYLEMNSLRSEDTAMY |
| | | YCASFWWDLDFDHWGQGVMVTVSSGGGGSGGGGSGG |
| | | GGSGGGGSDILVTQTPVSLPVSLGGHVSISCRSSQSLVRSE |
| | | GTTYFNWYLQKPGQSPQLLIYRVSNRFSGVPDRFSGSGSG |
| | | TDFTLKISRVEPEDLGVYYCLQSSHFPWTFGGGTKLELK |
| SEQ ID | Anti-CD3 | DIQMTQSPSSLSASVGDRVTITCHASQNIYVWLNWYQQK |
| NO: 801 | (4)/anti-CD28 | PGKAPKLLIYKASNLHTGVPSRFSGSGSGTDFTLTISSLQP |
| | (1) light chain | EDFATYYCQQGQTYPYTFGGGTKVEIKRTVAAPSVFIFPP |
| | | SDEQLKSGTASVVCLLNNFYPREAKVQWKVDNALQSGN |
| | | SQESVTEQDSKDSTYSLSSTLTLSKADYEKHKVYACEVT |
| | | HQGLSSPVTKSFNRGEC |
| SEQ ID | Anti-CD3 | QVQLVQSGAEVQRPGASVKVSCKASGYIFTEYYMYWVR |
| NO: 816 | (1)/anti-CD2 | QAPGQGLELVGRIDPEDGSIDYVEKFKKKVTLTADTSSST |
| | (1) heavy | AYMELSSLTSDDTAVYYCARGKFNYRFAYWGQGTLVTV |
| | chain | SSASTKGPSVFPLAPSSKSTSGGTAALGCLVKDYFPEPVT |
| | GADAPASK | VSWNSGALTSGVHTFPAVLQSSGLYSLSSVVTVPSSSLGT |
| | | QTYICNVNHKPSNTKVDKKVEPKSCDKTHTCPPCPAPELL |
| | | GAPSVFLFPPKPKDTLMISRTPEVTCVVVAVKHEDPEVKF |
| | | NWYVDGVEVHNAKTKPREEQYNSTYRVVSVLTVLHQD |
| | | WLNGKEYKCKVSNKALAAPIEKTISKAKGQPREPQVYTL |
| | | PPSRREEMKNQVSLTCLVKGFYPSDIAVEWESNGQPENN |
| | | YKTTPPVLDSDGSFFLYSKLTVDKSRWQQGNVFSCSVMH |
| | | EALHNHYTQKSLSLSPGGGGGSGGGGSGGGGSGGGGSQ |
| | | VQLVQSGGGVVQPGRSLRLSCKASGYTFTRYTMHWVRQ |
| | | APGKGLEWIGYINPSRGYTNYNQKVKDRFTISRDNSKNT |
| | | AFLQMDSLRPEDIGVYFCARYYDDHYSLDYWGQGTPVT |
| | | VSSGGGGSGGGGSGGGGSGGGGSDIQMTQSPSSLSASVG |
| | | DRVTITCSASSSVSYMNWYQQTPGKAPKRWIYDTSKLAS |
| | | GVPSRFSGSGSGTDYTFTISSLQPEDIATYYCQQWSSNPFT |
| | | FGQGTKLQIT |
| SEQ ID | Anti-CD3 | QVQLVQSGAEVQRPGASVKVSCKASGYIFTEYYMYWVR |
| NO: 817 | (1)/anti-CD2 | QAPGQGLELVGRIDPEDGSIDYVEKFKKKVTLTADTSSST |
| | (1) heavy | AYMELSSLTSDDTAVYYCARGKFNYRFAYWGQGTLVTV |
| | chain | SSASTKGPSVFPLAPSSKSTSGGTAALGCLVKDYFPEPVT |
| | LALAPG | VSWNSGALTSGVHTFPAVLQSSGLYSLSSVVTVPSSSLGT |
| | | QTYICNVNHKPSNTKVDKKVEPKSCDKTHTCPPCPAPEA |
| | | AGGPSVFLFPPKPKDTLMISRTPEVTCVVVDVSHEDPEVK |
| | | FNWYVDGVEVHNAKTKPREEQYNSTYRVVSVLTVLHQD |
| | | WLNGKEYKCKVSNKALGAPIEKTISKAKGQPREPQVYTL |
| | | PPSRREEMKNQVSLTCLVKGFYPSDIAVEWESNGQPENN |
| | | YKTTPPVLDSDGSFFLYSKLTVDKSRWQQGNVFSCSVMH |
| | | EALHNHYTQKSLSLSPGGGGGSGGGGSGGGGSGGGGSQ |
| | | VQLVQSGGGVVQPGRSLRLSCKASGYTFTRYTMHWVRQ |
| | | APGKGLEWIGYINPSRGYTNYNQKVKDRFTISRDNSKNT |
| | | AFLQMDSLRPEDTGVYFCARYYDDHYSLDYWGQGTPVT |
| | | VSSGGGGSGGGGSGGGGSGGGGSDIQMTQSPSSLSASVG |
| | | DRVTITCSASSSVSYMNWYQQTPGKAPKRWIYDTSKLAS |
| | | GVPSRFSGSGSGTDYTFTISSLQPEDIATYYCQQWSSNPFT |
| | | FGQGTKLQIT |
| SEQ ID | Fc | DKTHTCPPCPAPEAAGGPSVFLFPPKPKDTLMISRTPEVTC |
| NO: 802 | LALAPG | VVVDVSHEDPEVKFNWYVDGVEVHNAKTKPREEQYNST |
| | | YRVVSVLTVLHQDWLNGKEYKCKVSNKALGAPIEKTISK |
| | | AKGQPREPQVYTLPPSRREEMKNQVSLTCLVKGFYPSDIA |
| | | VEWESNGQPENNYKTTPPVLDSDGSFFLYSKLTVDKSRW |
| | | QQGNVFSCSVMHEALHNHYTQKSLSLSPGK |
| SEQ ID | Fc | DKTHTCPPCPAPELLGGPSVFLFPPKPKDTLMISRTPEVTC |
| NO: 803 | DAPA | VVVAVSHEDPEVKFNWYVDGVEVHNAKTKPREEQYNST |
| | | YRVVSVLTVLHQDWLNGKEYKCKVSNKALAAPIEKTISK |
| | | AKGQPREPQVYTLPPSREEMTKNQVSLTCLVKGFYPSDIA |
| | | VEWESNGQPENNYKTTPPVLDSDGSFFLYSKLTVDKSRW |
| | | QQGNVFSCSVMHEALHNHYTQKSLSLSPGK |
| SEQ ID | Fc | DKTHTCPPCPAPEAAGGPSVFLFPPKPKDTLMISRTPEVTC |
| NO: 804 | LALASKPA | VVVDVKHEDPEVKFNWYVDGVEVHNAKTKPREEQYNS |
| | | TYRVVSVLTVLHQDWLNGKEYKCKVSNKALAAPIEKTIS |
| | | KAKGQPREPQVYTLPPSREEMTKNQVSLTCLVKGFYPSDI |
| | | AVEWESNGQPENNYKTTPPVLDSDGSFFLYSKLTVDKSR |
| | | WQQGNVFSCSVMHEALHNHYTQKSLSLSPGK |
| SEQ ID | Fc | DKTHTCPPCPAPELLGAPSVFLFPPKPKDTLMISRTPEVTC |
| NO: 805 | GADAPASK | VVVAVKHEDPEVKFNWYVDGVEVHNAKTKPREEQYNS |
| | | TYRVVSVLTVLHQDWLNGKEYKCKVSNKALAAPIEKTIS |
| | | KAKGQPREPQVYTLPPSRREEMKNQVSLTCLVKGFYPSDI |
| | | AVEWESNGQPENNYKTTPPVLDSDGSFFLYSKLTVDKSR |
| | | WQQGNVFSCSVMHEALHNHYTQKSLSLSPGK |

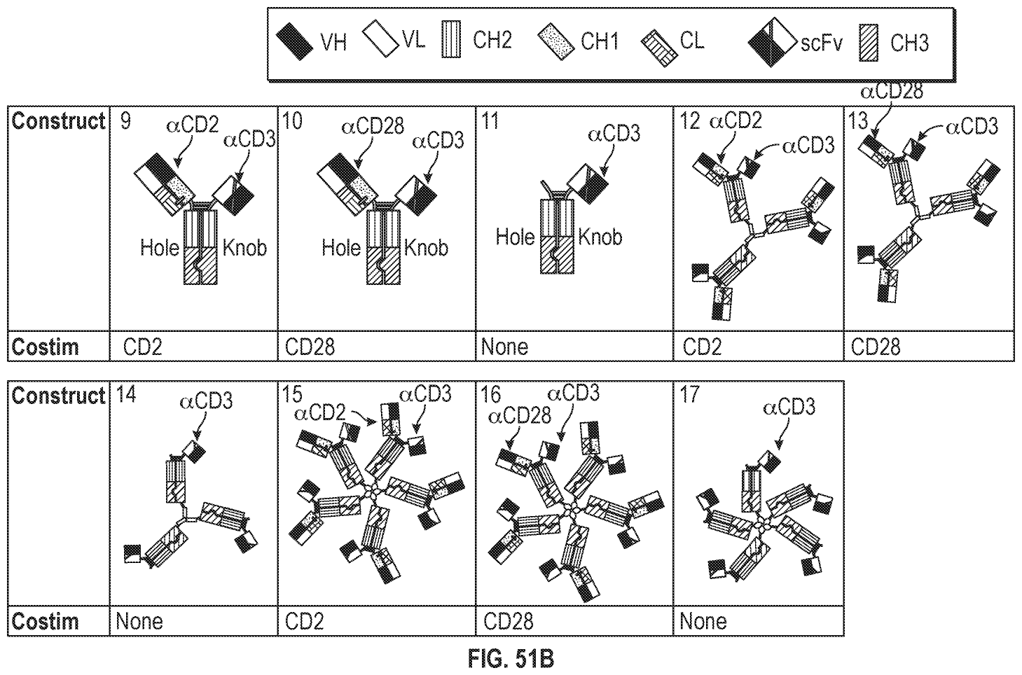

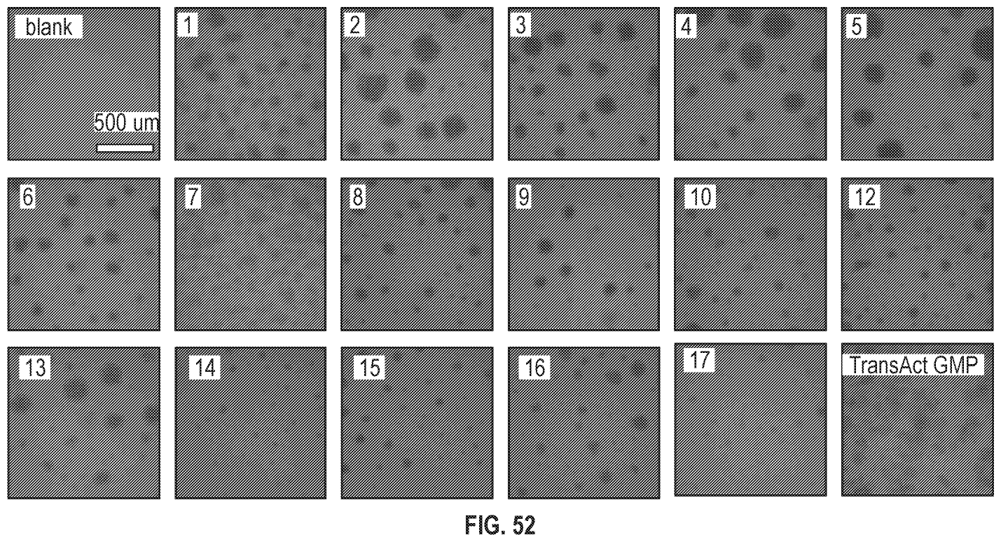

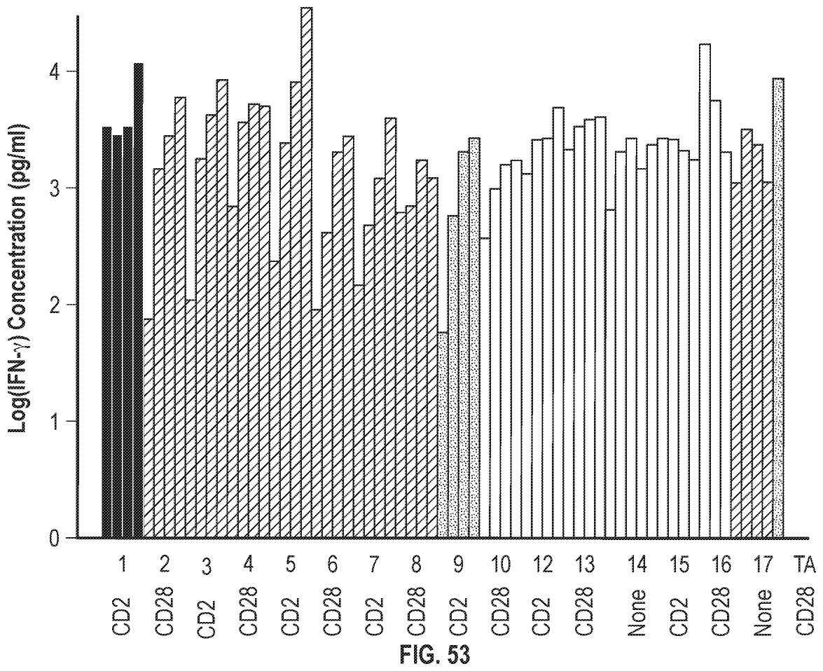

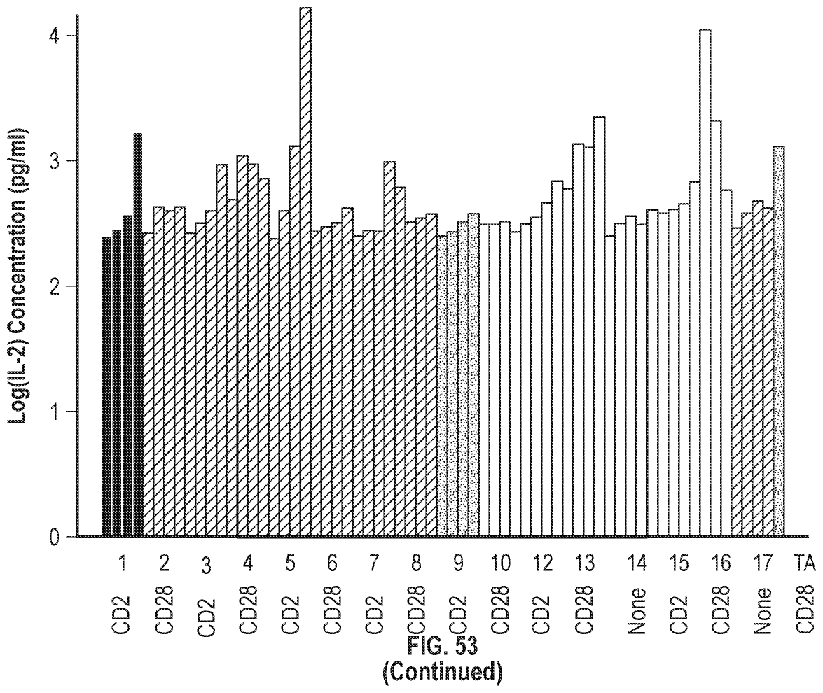

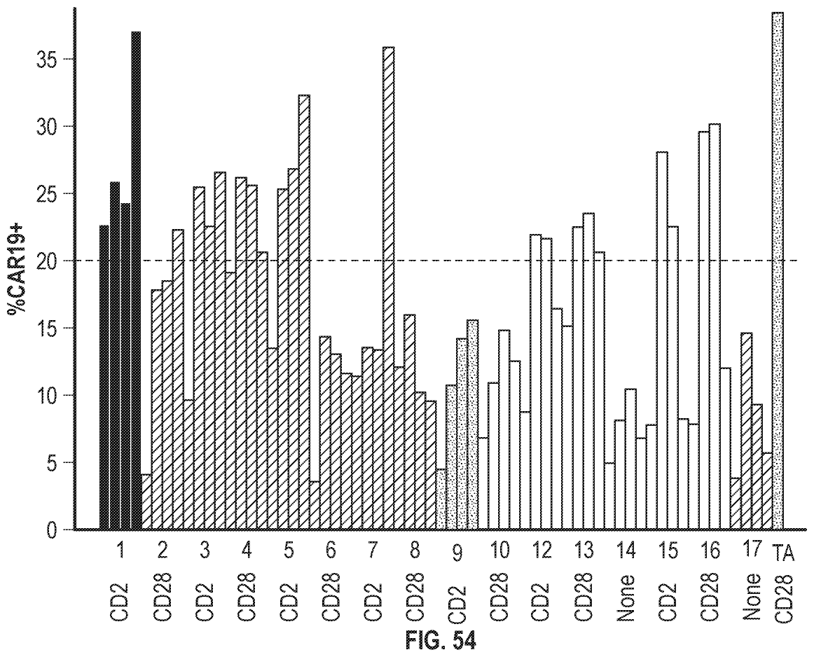

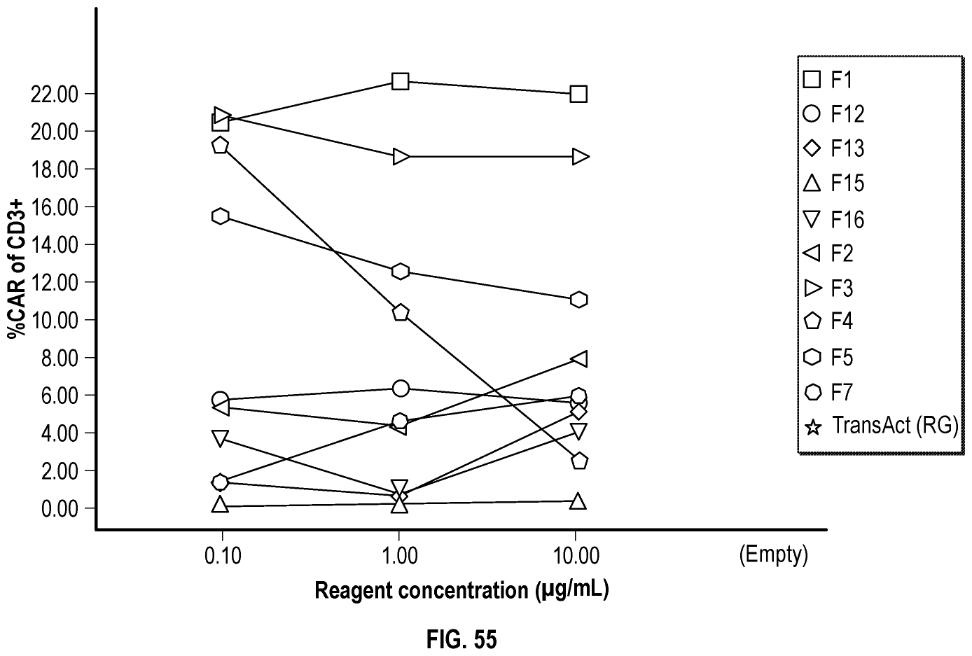

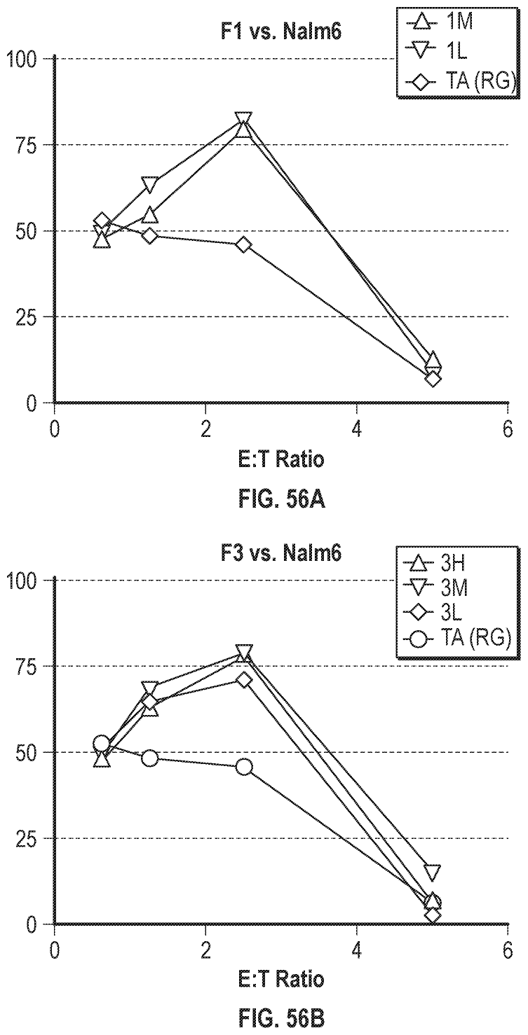

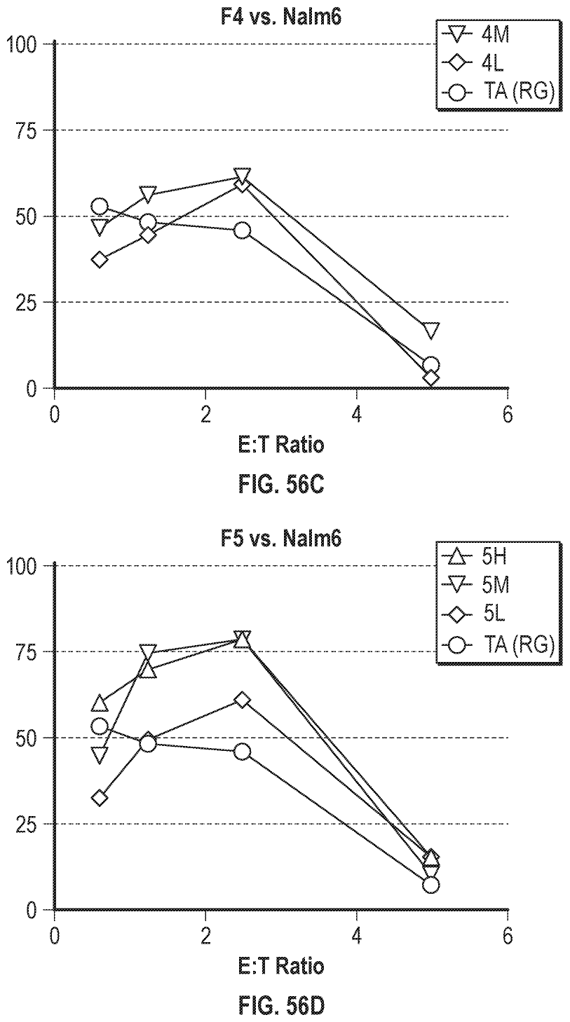

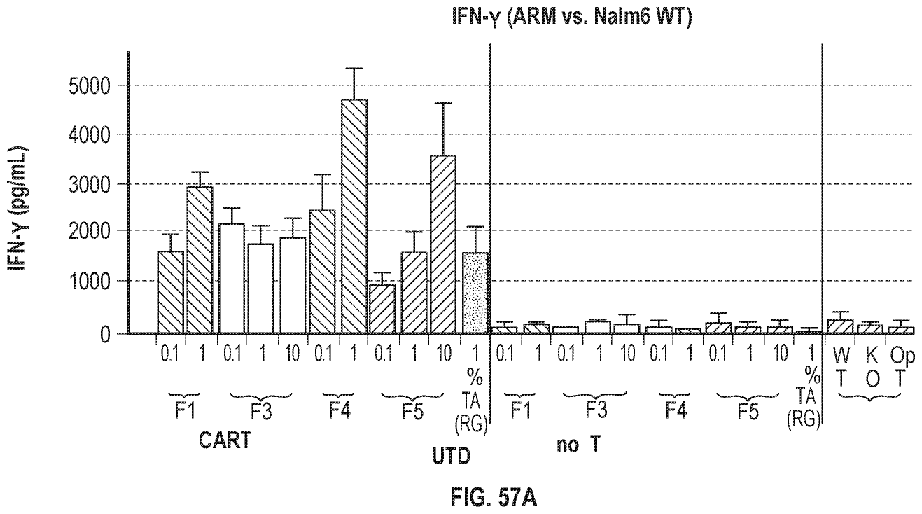

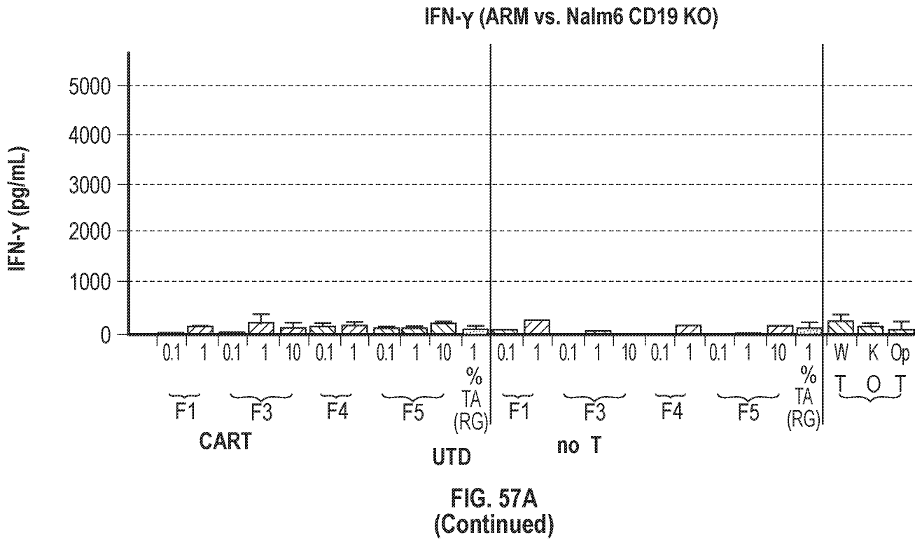

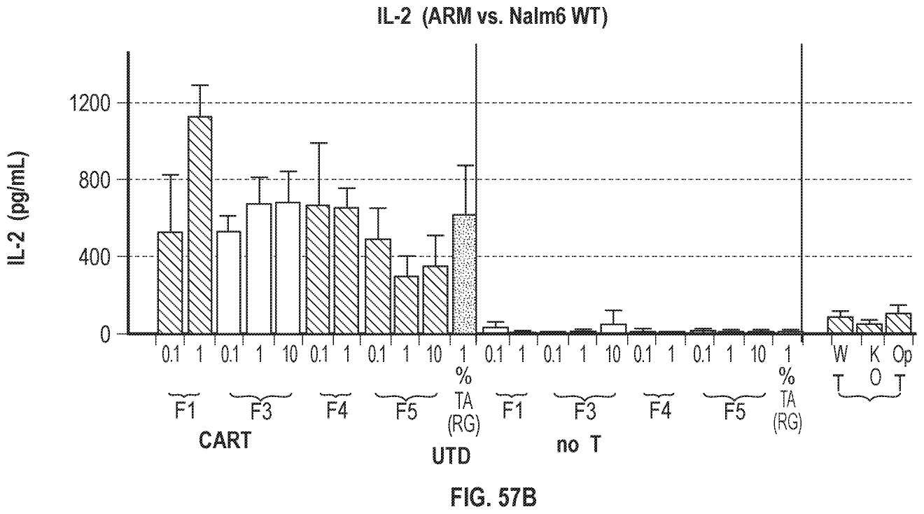

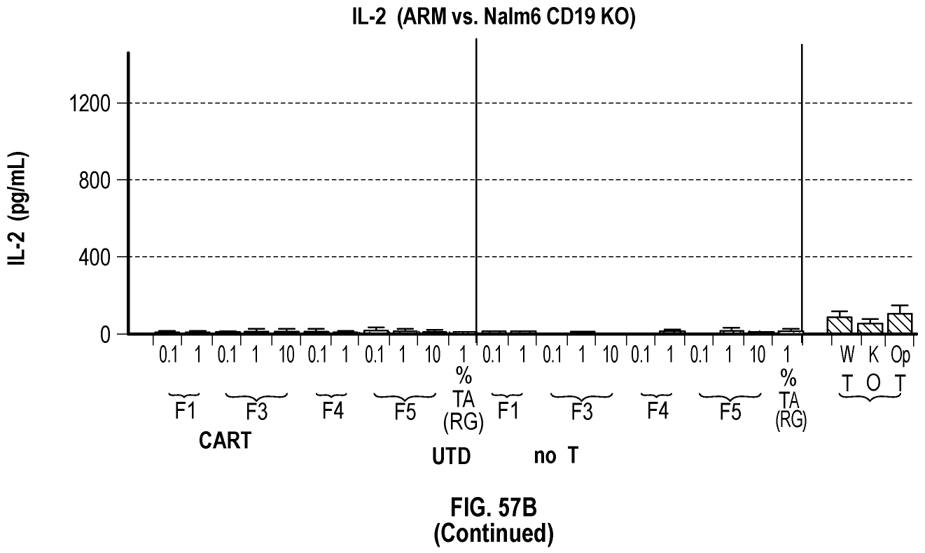

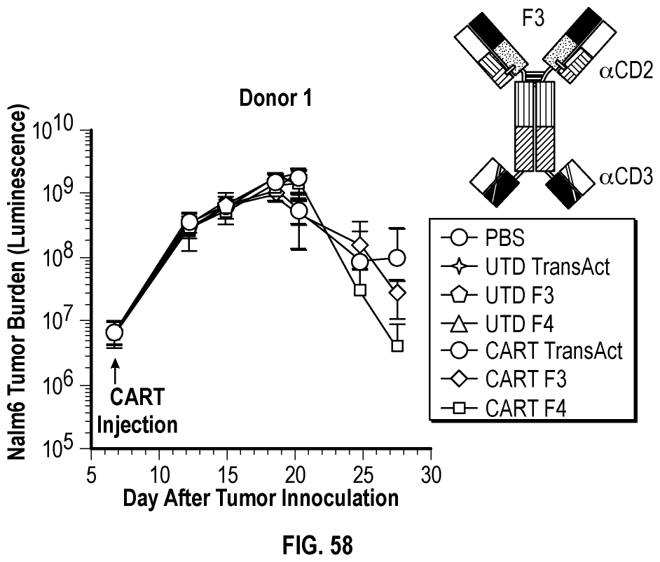

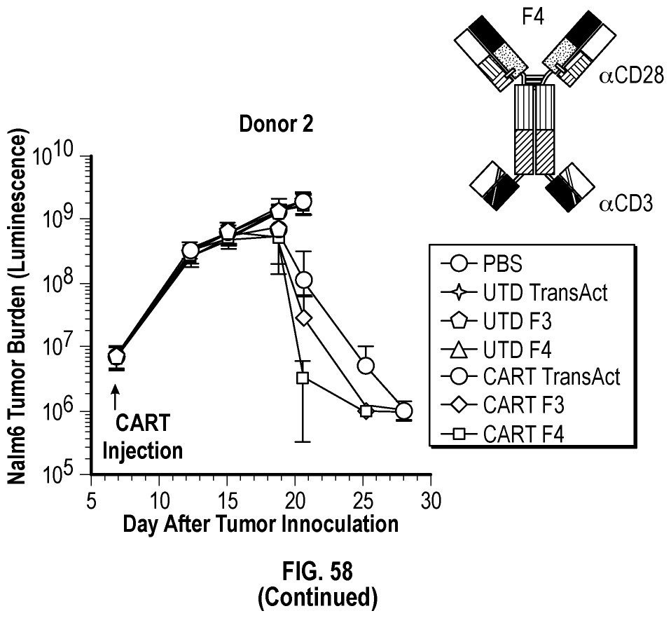

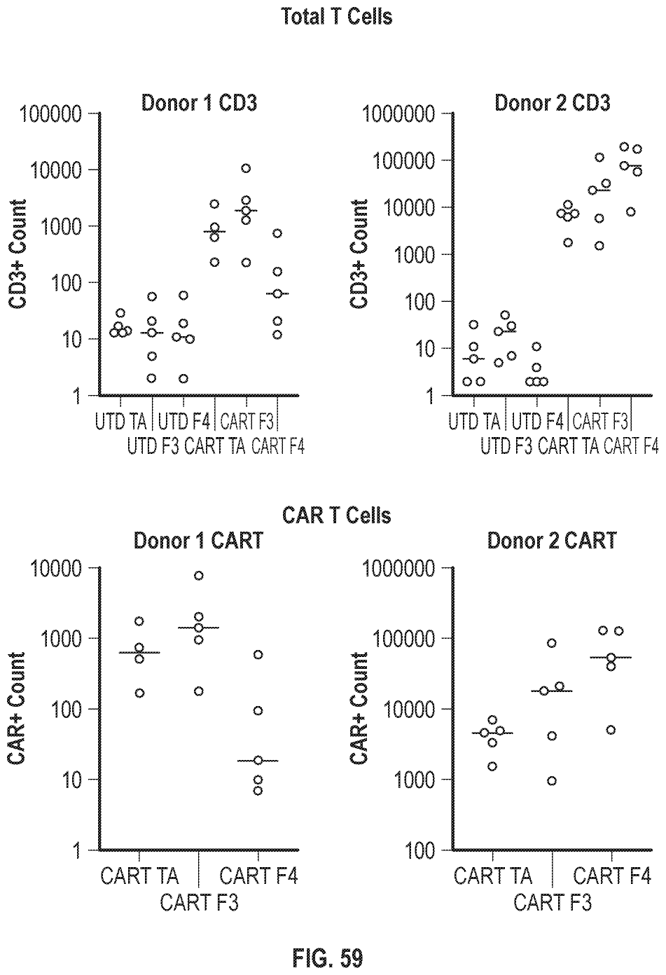

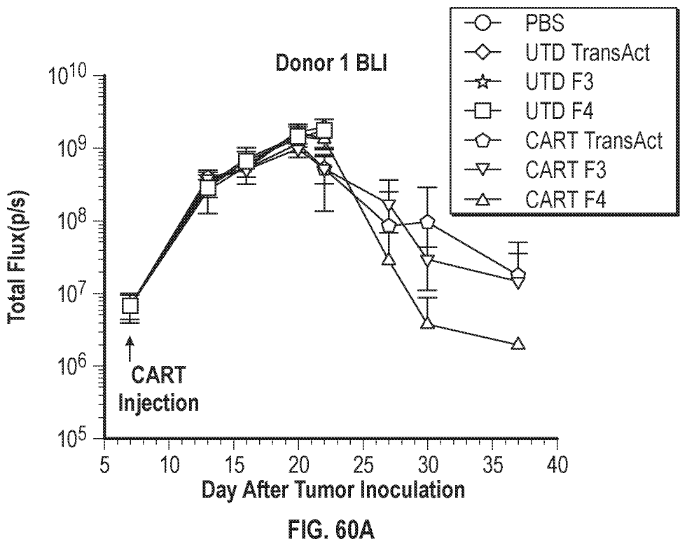

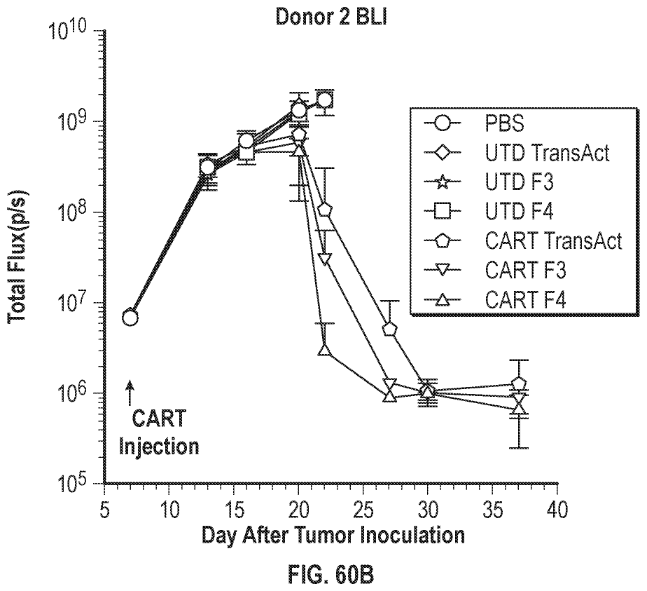

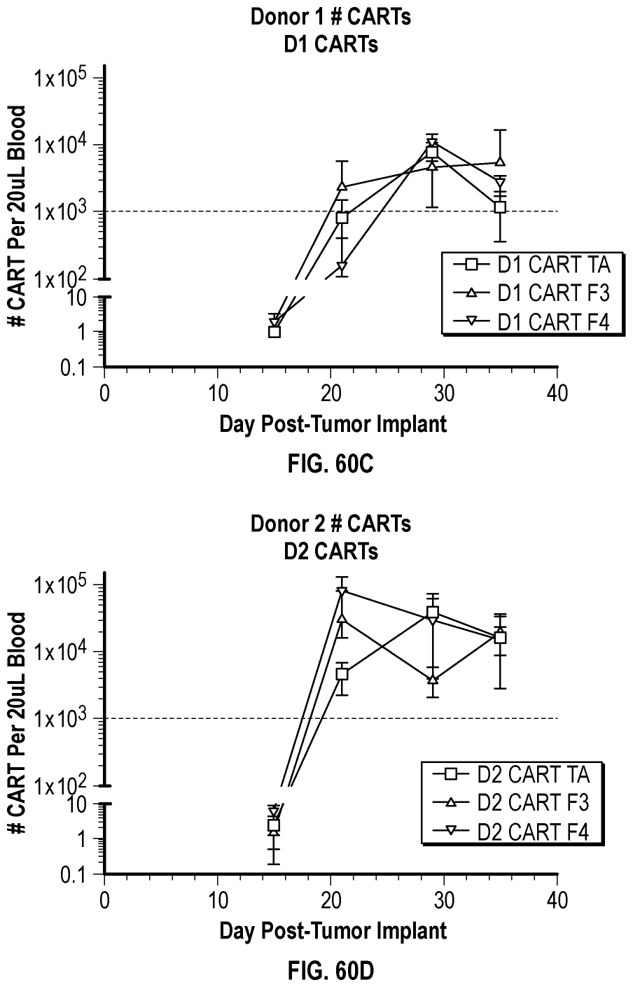

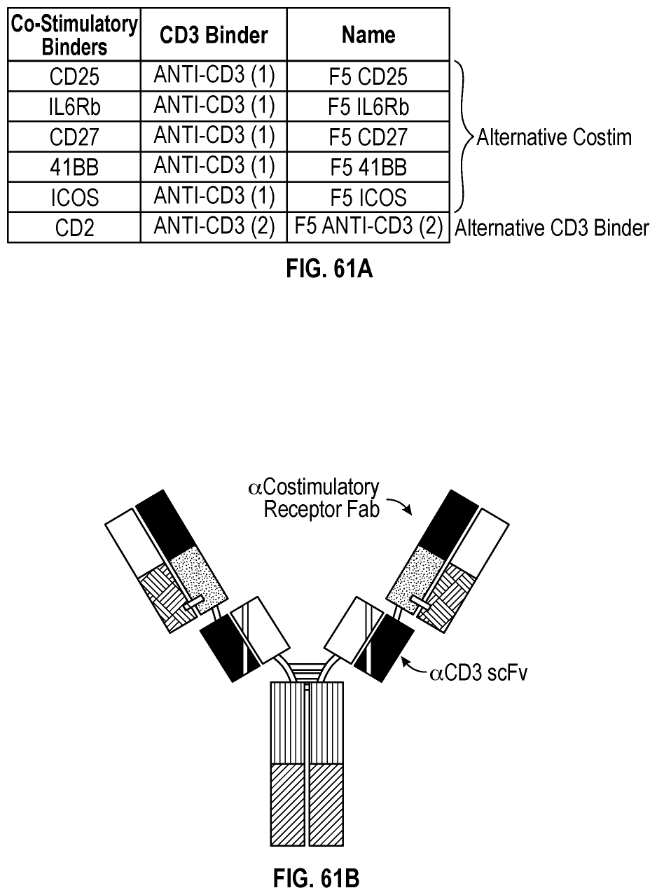

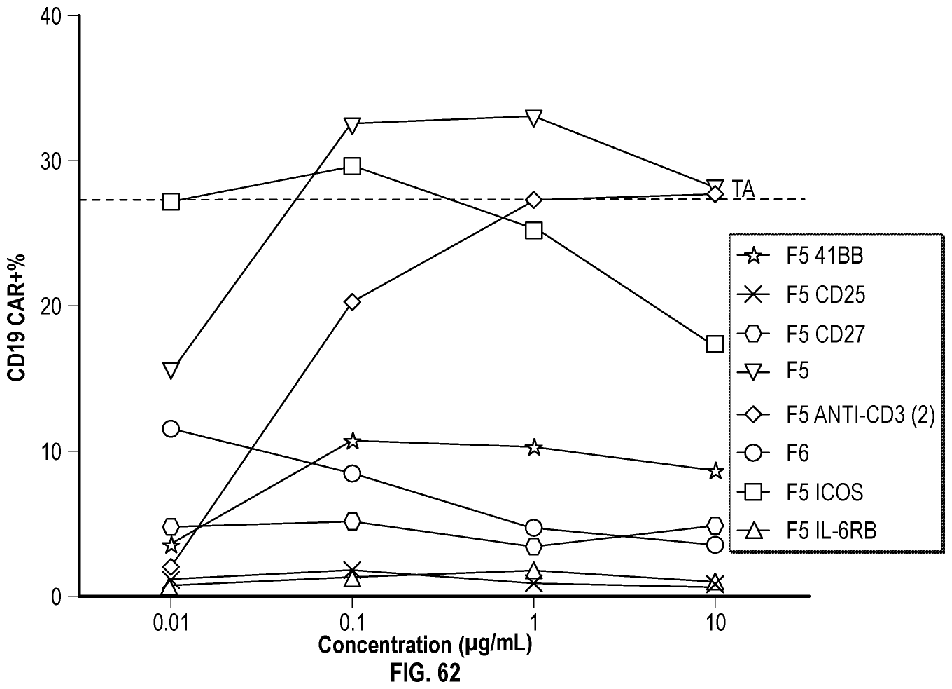

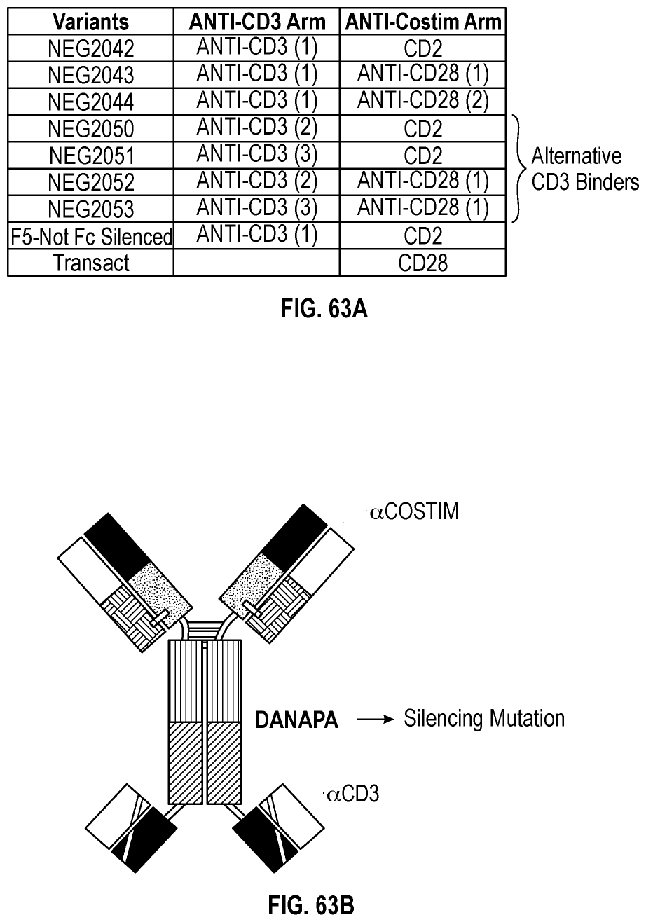

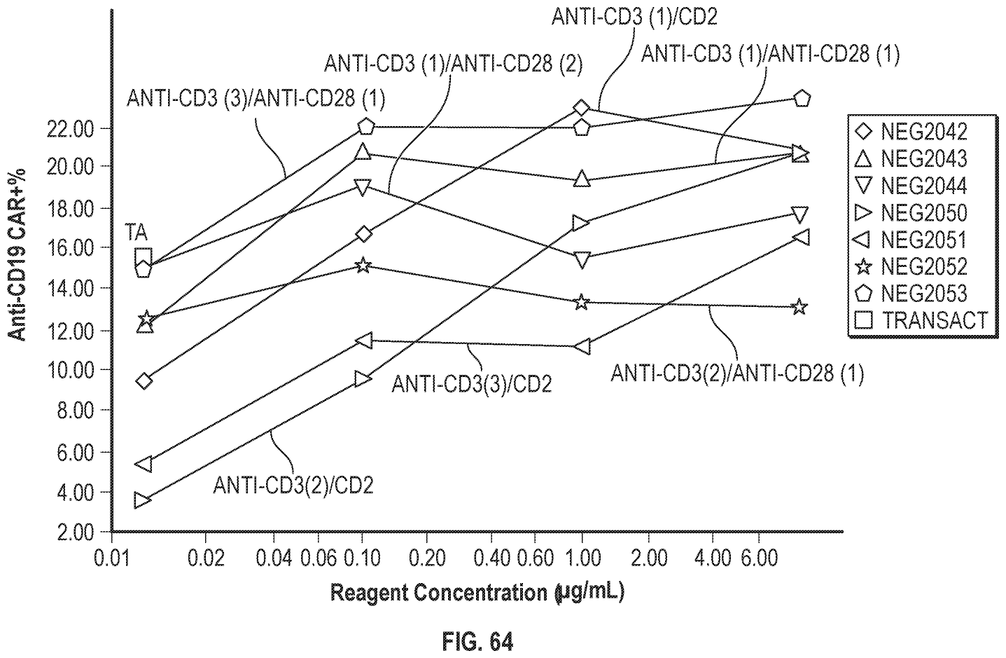

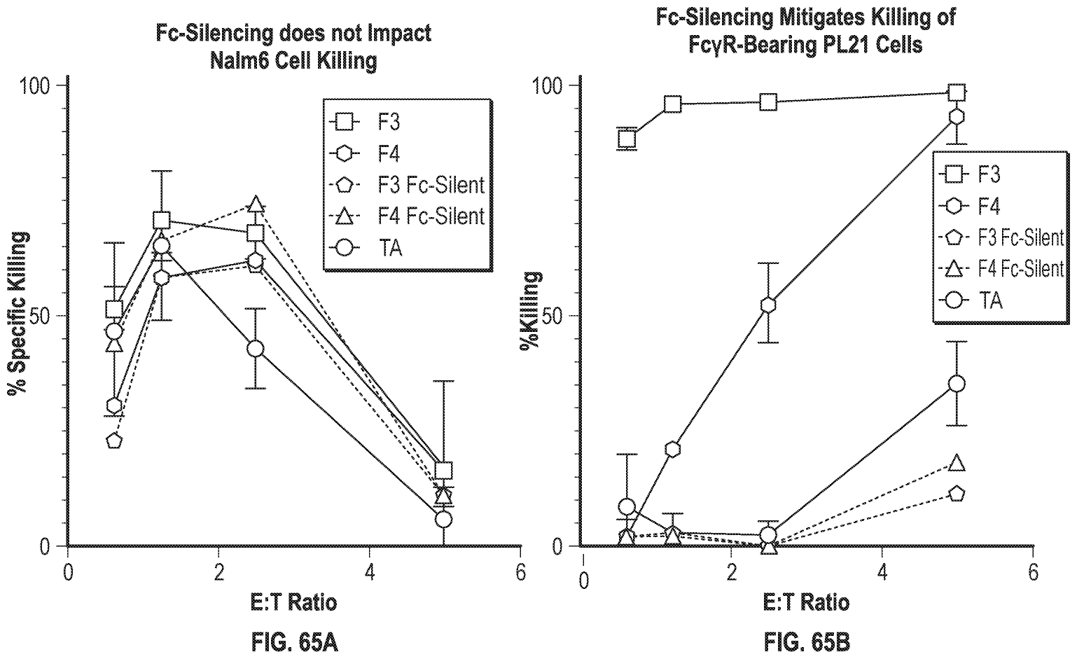

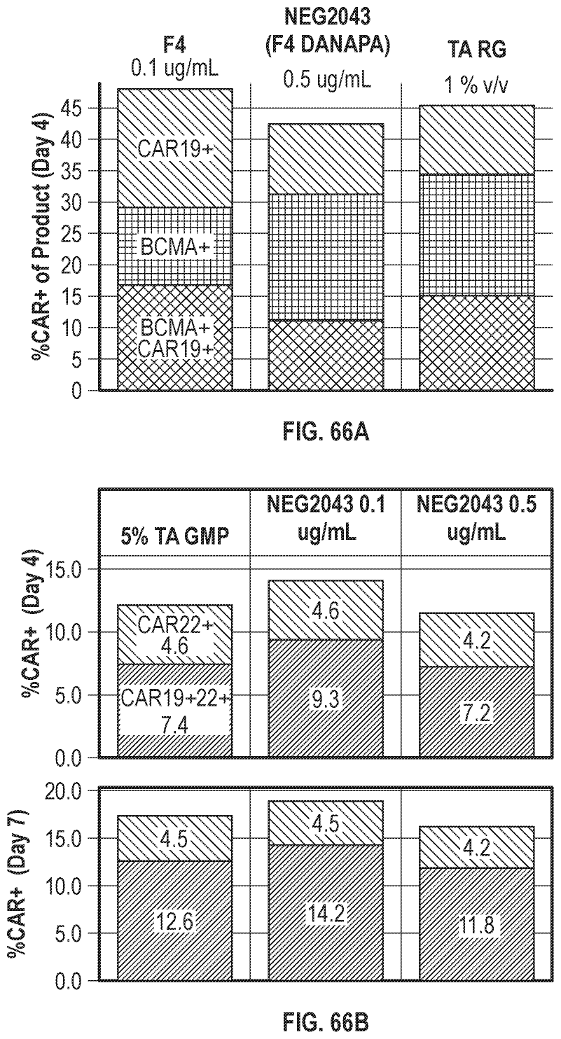

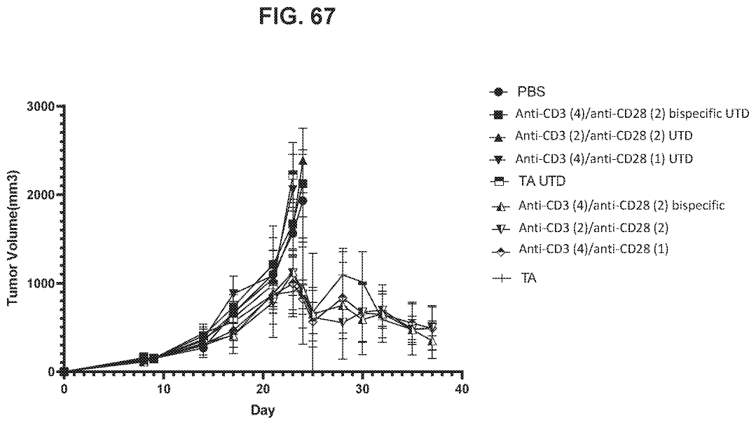

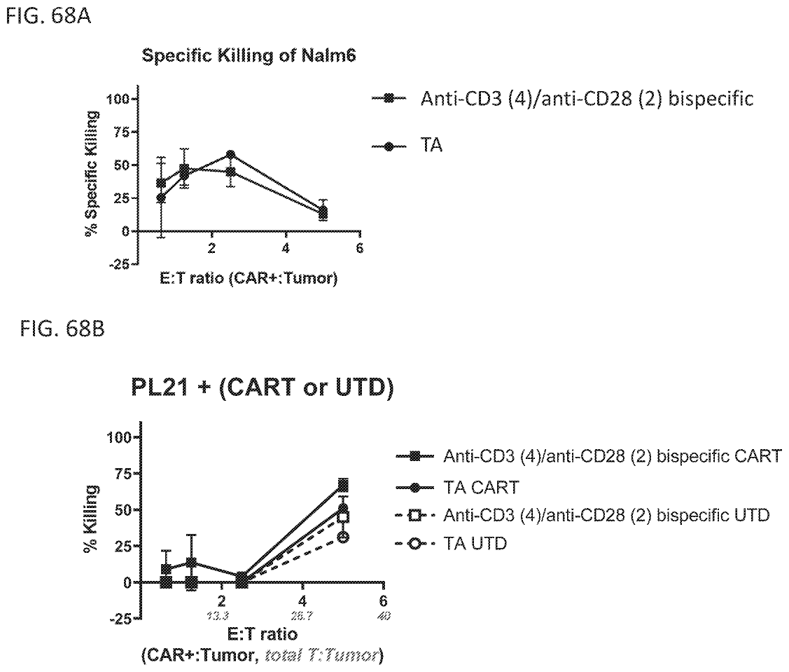

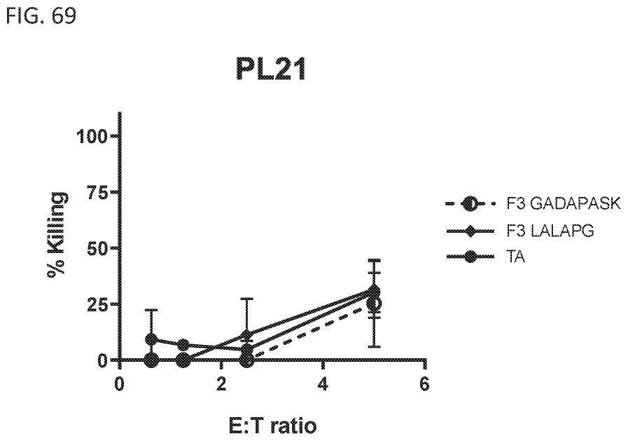

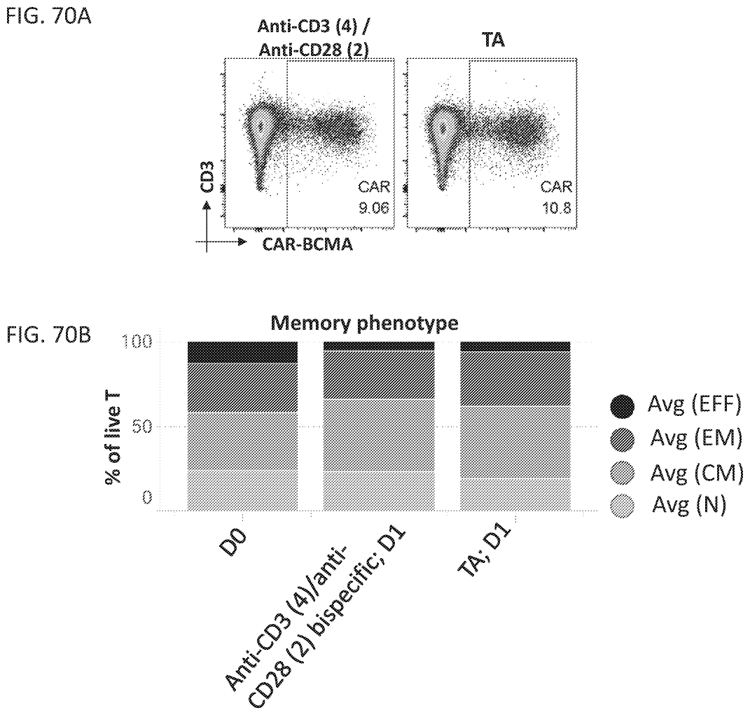

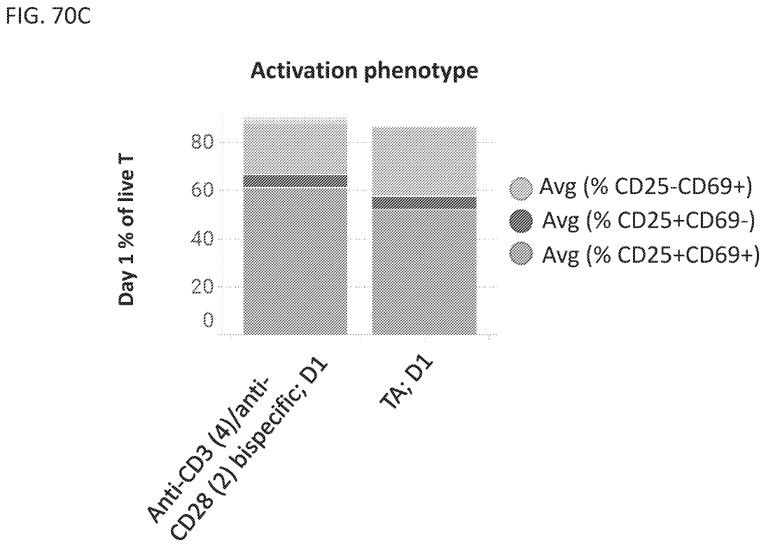

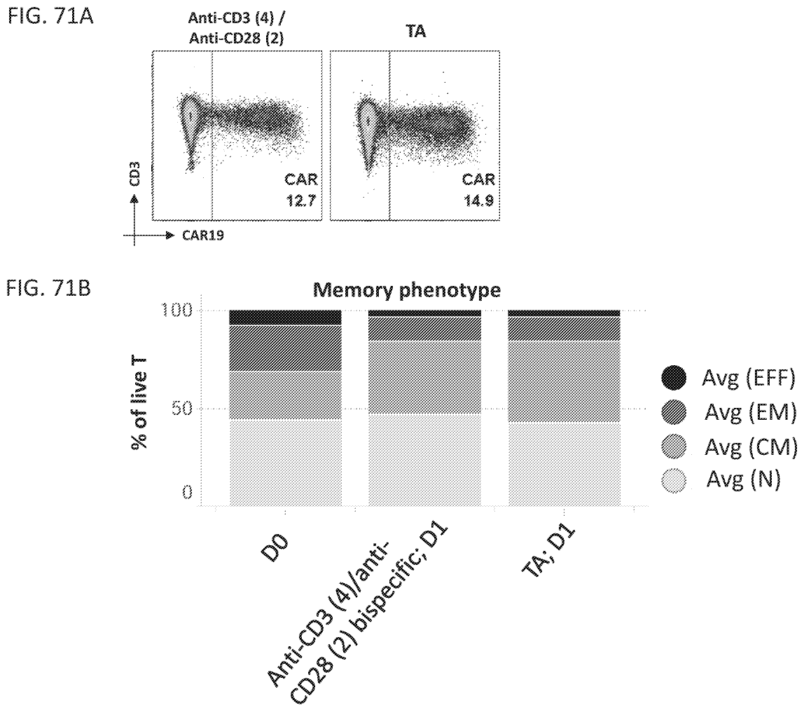

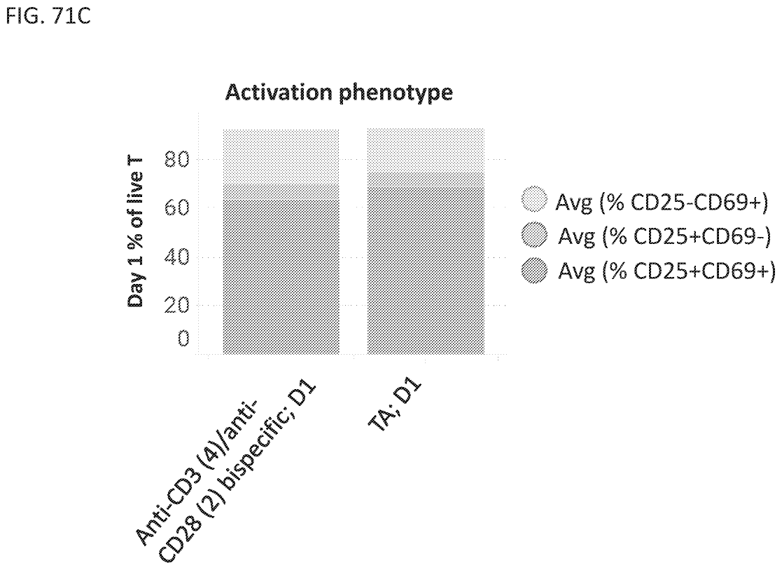

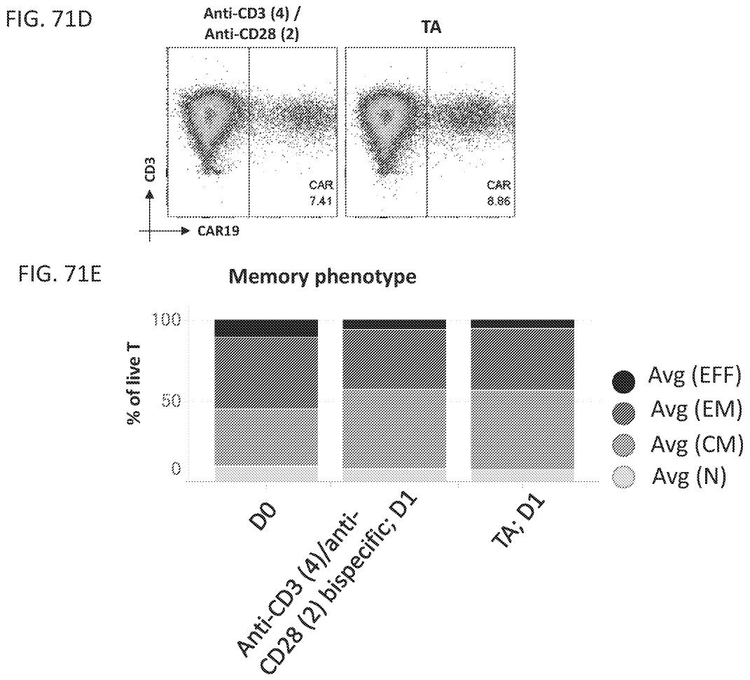

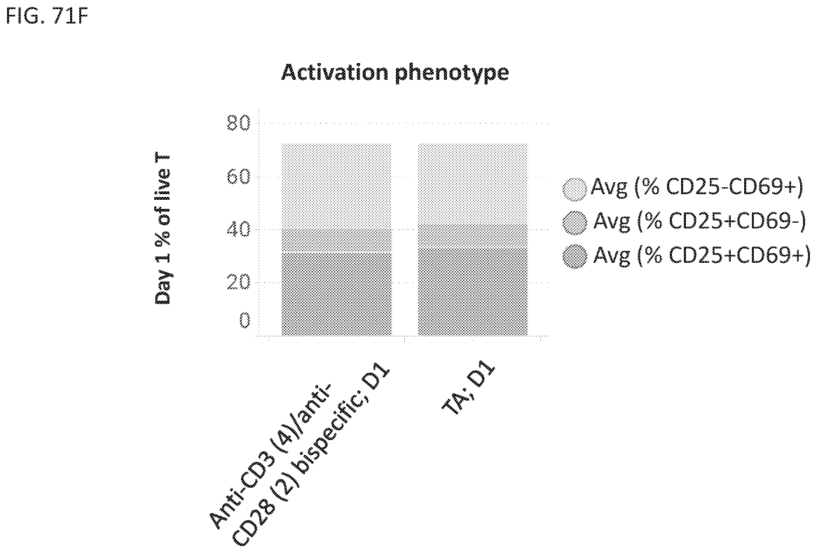

In some embodiments, the multispecific binding molecule comprises a bispecific antibody. In some embodiments, the bispecific antibody is configured in any one of the schema provided in FIG. 50A, FIGS. 51A-51B, and FIGS. 61A-61B, and FIGS. 63A-63B. In some embodiments, the bispecific antibody is monovalent or bivalent. In some embodiments, the bispecific antibody comprises an Fc region. In some embodiments, the Fc region of the bispecific antibody is silenced. In some embodiments, the Fc region of the bispecific antibody is silenced by a combination of amino acid substitutions selected from the group consisting of LALA, DAPA, DANAPA, LALADANAPS, LALAGA, LALASKPA, DAPASK, GADAPA, GADAPASK, LALAPG, and LALAPA.

In some embodiments, the multispecific binding molecule comprises a plurality of bispecific antibodies. In some embodiments, one or more of the plurality of bispecific antibodies is monovalent. In some embodiments, one or more of the plurality of bispecific antibodies comprises an Fc region. In some embodiments, the Fc region of the one or more of the plurality of bispecific antibodies is silenced. In some embodiments, one or more of the plurality of bispecific antibodies are conjugated together into a multimer. In some embodiments, the multimer is configured in any one of the schema provided in FIG. 50B and FIG. 51B.

In some embodiments, a multispecific binding molecule described herein comprises an Fc region, e.g., wherein the Fc region is Fc silent. In some embodiments, the Fc region comprises a mutation at one or more of (e.g., all of) D265, N297, and P329, wherein the amino acid residues are numbered according to the EU numbering system. In some embodiments, the Fc region comprises the mutations D265A, N297A, and P329A (DANAPA), wherein the amino acid residues are numbered according to the EU numbering system. In some embodiments, the Fc region comprises a mutation at one or more of (e.g., all of) L234A, L235A, and G237A, wherein the amino acid residues are numbered according to the EU numbering system. In some embodiments, the Fc region comprises the mutations L234A, L235A, and G237A (LALAGA), wherein the amino acid residues are numbered according to the EU numbering system. In some embodiments, the Fc region comprises a mutation at one or more of (e.g., all of) L234A, L235A, S267K, and P329A, wherein the amino acid residues are numbered according to the EU numbering system. In some embodiments, the Fc region comprises the mutations L234A, L235A, S267K, and P329A (LALASKPA), wherein the amino acid residues are numbered according to the EU numbering system. In some embodiments, the Fc region comprises a mutation at one or more of (e.g., all of) D265A, P329A, and S267K, wherein the amino acid residues are numbered according to the EU numbering system. In some embodiments, the Fc region comprises the mutations D265A, P329A, and S267K (DAPASK), wherein the amino acid residues are numbered according to the EU numbering system. In some embodiments, the Fc region comprises a mutation at one or more of (e.g., all of) G237A, D265A, and P329A, wherein the amino acid residues are numbered according to the EU numbering system. In some embodiments, the Fc region comprises the mutations G237A, D265A, and P329A (GADAPA), wherein the amino acid residues are numbered according to the EU numbering system. In some embodiments, the Fc region comprises a mutation at one or more of (e.g., all of) G237A, D265A, P329A, and S267K, wherein the amino acid residues are numbered according to the EU numbering system. In some embodiments, the Fc region comprises the mutations G237A, D265A, P329A, and S267K (GADAPASK), wherein the amino acid residues are numbered according to the EU numbering system. In some embodiments, the Fc region comprises a mutation at one or more of (e.g., all of) L234A, L235A, and P329G, wherein the amino acid residues are numbered according to the EU numbering system. In some embodiments, the Fc region comprises the mutations L234A, L235A, and P329G (LALAPG), wherein the amino acid residues are numbered according to the EU numbering system. In some embodiments, the Fc region comprises a mutation at one or more of (e.g., all of) L234A, L235A, and P329A, wherein the amino acid residues are numbered according to the EU numbering system. In some embodiments, the Fc region comprises the mutations L234A, L235A, and P329A (LALAPA), wherein the amino acid residues are numbered according to the EU numbering system.

In some embodiments, a multispecific binding molecule described herein comprises a first binding domain and a second binding domain. For instance, the first binding domain may be an anti-CD3 binding domain and the second binding domain may be a costimulatory molecule binding domain, or the first binding domain may be a costimulatory molecule binding domain and the second binding domain may be an anti-CD3 binding domain. In some embodiments, the costimulatory molecule binding domain binds to CD2, CD28, CD25, CD27, IL6Rb, ICOS, or 41BB. In some embodiments, the costimulatory molecule binding domain activates CD2, CD28, CD25, CD27, IL6Rb, ICOS, or 41BB. In some embodiments, a multispecific binding molecule described herein comprises an Fc region that is mutated to have reduced binding to Fc receptor or reduced ADCC, ADCP, or CDC activity, e.g., an Fc region comprising the mutations D265A, N297A, and P329A (DANAPA); L234A, L235A, and G237A (LALAGA); L234A, L235A, S267K, and P329A (LALASKPA); D265A, P329A, and S267K (DAPASK); G237A, D265A, and P329A (GADAPA); G237A, D265A, P329A, and S267K (GADAPASK); L234A, L235A, and P329G (LALAPG); or L234A, L235A, and 20) P329A (LALAPA), wherein the amino acid residues are numbered according to the EU numbering system.

In some embodiments, the first binding domain (e.g., an scFv) is N-terminal of the VH of the second binding domain (e.g., a Fab fragment), e.g., linked via a peptide linker. In some embodiments, the multispecific binding molecule further comprises one or more of (e.g., all of) a CH1, CH2, and CH3, e.g., in order from N-terminal to C-terminal. In some embodiments, a polypeptide of the multispecific binding molecule comprises the following sequences, from N-terminal to C-terminal: VH of the first binding domain, first peptide linker (e.g., a (G4S) 4 linker), VL of first binding domain, second peptide linker (e.g., a (G4S) 4 linker), VH of the second binding domain, CH1, CH2, and CH3. In some embodiments, a polypeptide of the multispecific binding molecule comprises the following sequences: from N-terminal to C-terminal: VL of the second binding domain and CL. In some embodiments, the multispecific binding molecule comprises an Fc region that is mutated to have reduced binding to Fc receptor or reduced ADCC, ADCP, or CDC activity, e.g., an Fc region comprising the mutations D265A, N297A, and P329A (DANAPA); L234A, L235A, and G237A (LALAGA); L234A, L235A, S267K, and P329A (LALASKPA); D265A, P329A, and S267K (DAPASK); G237A, D265A, and P329A (GADAPA); G237A, D265A, P329A, and S267K (GADAPASK); L234A, L235A, and P329G (LALAPG); or L234A, L235A, and P329A (LALAPA), wherein the amino acid residues are numbered according to the EU numbering system. In some embodiments, the first binding fragment comprises an anti-CD3 binding domain, e.g., an anti-CD3 scFv, e.g., comprising an anti-CD3 sequence disclosed in Table 27. In some embodiments, the second binding domain comprises a costimulatory molecule binding domain, e.g., an anti-CD2 binding domain, e.g., an anti-CD2 Fab, e.g., comprising an anti-CD2 sequence disclosed in Table 27. In some embodiments, the second binding domain comprises a costimulatory molecule binding domain, e.g., an anti-CD28 binding domain, e.g., an anti-CD28 Fab, e.g., comprising an anti-CD28 sequence disclosed in Table 27. In some embodiments, the first binding fragment comprises a costimulatory molecule binding domain, e.g., an anti-CD2 or anti-CD28 binding domain, e.g., an anti-CD2 or anti-CD28 scFv, e.g., comprising an anti-CD2 or anti-CD28 sequence disclosed in Table 27. In some embodiments, the second binding domain comprises an anti-CD3 binding domain, e.g., an anti-CD3 Fab, e.g., comprising an anti-CD3 sequence disclosed in Table 27. Examples of such multispecific binding molecules are depicted as the top left construct in FIG. 50A; Construct 1 or Construct 2 in FIG. 51A; and Construct 1 or Construct 2 in Table 28.

In some embodiments, the first binding domain (e.g., a Fab fragment) is N-terminal to a second binding domain (e.g., an scFv), e.g., wherein an Fc region is situated between the first and second binding domain. In some embodiments, the Fc region is mutated to have reduced binding to Fc receptor or reduced ADCC, ADCP, or CDC activity, e.g., an Fc region comprising the mutations D265A, N297A, and P329A (DANAPA); L234A, L235A, and G237A (LALAGA); L234A, L235A, S267K, and P329A (LALASKPA); D265A, P329A, and S267K (DAPASK); G237A, D265A, and P329A (GADAPA); G237A, D265A, P329A, and S267K (GADAPASK); L234A, L235A, and P329G (LALAPG); or L234A, L235A, and P329A (LALAPA), wherein the amino acid residues are numbered according to the EU numbering system. In some embodiments, the multispecific binding molecule further comprises one or more of (e.g., all of) a CH1, CH2, and CH3, e.g., in order from N-terminal to C-terminal. In some embodiments, a polypeptide of the multispecific binding molecule comprises the following sequences, from N-terminal to C-terminal: VH of the first binding domain, CH1, CH2, CH3, first peptide linker (e.g., a (G4S) 4 linker), VH of second binding domain, second peptide linker (e.g., a (G4S) 4 linker), and VL of the second binding domain. In some embodiments, a polypeptide of the multispecific binding molecule comprises the following sequences: from N-terminal to C-terminal: VL of the first binding domain and CL. In some embodiments, the first binding domain comprises a costimulatory molecule binding domain, e.g., an anti-CD2 binding domain, e.g., an anti-CD2 Fab, e.g., comprising an anti-CD2 sequence disclosed in Table 27. In some embodiments, the first binding domain comprises a costimulatory molecule binding domain, e.g., an anti-CD28 binding domain, e.g., an anti-CD28 Fab, e.g., comprising an anti-CD28 sequence disclosed in Table 27, e.g., anti-CD28 (1) or anti-CD28 (2). In some embodiments, the second binding domain comprises an anti-CD3 binding domain, e.g., an anti-CD3 scFv, e.g., comprising an anti-CD3 sequence disclosed in Table 27, e.g., anti-CD3 (1), anti-CD3 (2), anti-CD3 (3), or anti-CD3 (4). In some embodiments, the first binding domain comprises an anti-CD3 binding domain, e.g., an anti-CD3 Fab, e.g., comprising an anti-CD3 sequence disclosed in Table 27, e.g., anti-CD3 (1), anti-CD3 (2), anti-CD3 (3), or anti-CD3 (4). In some embodiments, the second binding domain comprises a costimulatory molecule binding domain, e.g., an anti-CD2 or anti-CD28 binding domain, e.g., an anti-CD2 or anti-CD28 scFv, e.g., comprising an anti-CD2 or anti-CD28 sequence disclosed in Table 27. Examples of such multispecific binding molecules are depicted as the second construct from the left in the top row of FIG. 50A; Construct 3 or Construct 4 in FIG. 51A; and Construct 3, Construct 4, Anti-CD3 (4)/anti-CD28 (2) bispecific construct, Anti-CD3 (2)/anti-CD28 (2) construct, or Anti-CD3 (4)/anti-CD28 (1) construct in Table 28.

In some embodiments, the first binding domain (e.g., a Fab fragment) is N terminal to a second binding domain (e.g., a scFv), e.g., via a peptide linker. In some embodiments, the multispecific binding molecule further comprises one or more of (e.g., all of) a CH1, CH2, and CH3, e.g., in order from N-terminal to C-terminal. In some embodiments, a polypeptide of the multispecific binding molecule comprises the following sequences, from N-terminal to C-terminal: VH of the first binding domain, CH1, first peptide linker (e.g., a (G4S) 2 linker), VH of the second binding domain, second peptide linker (e.g., a (G4S) 4 linker), VL of the second binding domain, third peptide linker (e.g., a (G4S) 4 linker), CH2, and CH3. In some embodiments, a polypeptide of the multispecific binding molecule comprises the following sequences: from N-terminal to C-terminal: VL of the first binding domain and CL. In some embodiments, the multispecific binding molecule comprises an Fc region that is mutated to have reduced binding to Fc receptor or reduced ADCC, ADCP, or CDC activity, e.g., an Fc region comprising the mutations D265A, N297A, and P329A (DANAPA); L234A, L235A, and G237A (LALAGA); L234A, L235A, S267K, and P329A (LALASKPA); D265A, P329A, and S267K (DAPASK); G237A, D265A, and P329A (GADAPA); G237A, D265A, P329A, and S267K (GADAPASK); L234A, L235A, and P329G (LALAPG); or L234A, L235A, and P329A (LALAPA), wherein the amino acid residues are numbered according to the EU numbering system. In some embodiments, the first binding domain comprises a costimulatory molecule binding domain, e.g., an anti-CD2 binding domain, e.g., an anti-CD2 Fab, e.g., comprising an anti-CD2 sequence disclosed in Table 27. In some embodiments, the first binding domain comprises a costimulatory molecule binding domain, e.g., an anti-CD28 binding domain, e.g., an anti-CD28 Fab, e.g., comprising an anti-CD28 sequence disclosed in Table 27, e.g., anti-CD28 (1) or anti-CD28 (2). In some embodiments, the first binding domain comprises a costimulatory molecule binding domain, e.g., an anti-CD25 binding domain (for example, an anti-CD25 Fab), an anti-CD27 binding domain (for example, an anti-CD27 Fab), an anti-IL6Rb binding domain (for example, an anti-IL6Rb Fab), an anti-ICOS binding domain (for example, an anti-ICOS Fab), or an anti-41BB binding domain (for example, an anti-41BB Fab). In some embodiments, the second binding domain comprises an anti-CD3 binding domain, e.g., an anti-CD3 scFv, e.g., comprising an anti-CD3 sequence disclosed in Table 27, e.g., anti-CD3 (1), anti-CD3 (2), anti-CD3 (3), or anti-CD3 (4). In some embodiments, the first binding domain comprises an anti-CD3 binding domain, e.g., an anti-CD3 Fab, e.g., comprising an anti-CD3 sequence disclosed in Table 27, e.g., anti-CD3 (1), anti-CD3 (2), anti-CD3 (3), or anti-CD3 (4). In some embodiments, the second binding domain comprises a costimulatory molecule binding domain, e.g., an anti-CD2 binding domain (for example, an anti-CD2 scFv), an anti-CD28 binding domain (for example, an anti-CD28 scFv), an anti-CD25 binding domain (for example, an anti-CD25 scFv), an anti-CD27 binding domain (for example, an anti-CD27 scFv), an anti-IL6Rb binding domain (for example, an anti-IL6Rb scFv), an anti-ICOS binding domain (for example, an anti-ICOS scFv), or an anti-41BB binding domain (for example, an anti-41BB scFv). Examples of such multispecific binding molecules are depicted as the third construct from the left in the top row of FIG. 50A; Construct 5 or Construct 6 in FIG. 51A; and Construct 5 or Construct 6 in Table 28.

In some embodiments, the first binding domain (e.g., an scFv) is N-terminal to a second binding domain (e.g., a Fab fragment), e.g., wherein an Fc region is situated between the first and second binding domain. In some embodiments, the Fc region is mutated to have reduced binding to Fc receptor or reduced ADCC, ADCP, or CDC activity, e.g., an Fc region comprising the mutations D265A, N297A, and P329A (DANAPA); L234A, L235A, and G237A (LALAGA); L234A, L235A, S267K, and P329A (LALASKPA); D265A, P329A, and S267K (DAPASK); G237A, D265A, and P329A (GADAPA); G237A, D265A, P329A, and S267K (GADAPASK); L234A, L235A, and P329G (LALAPG); or L234A, L235A, and P329A (LALAPA), wherein the amino acid residues are numbered according to the EU numbering system. In some embodiments, the multispecific binding molecule further comprises one or more of (e.g., all of) a CH2, CH3, and CH1, e.g., in order from N-terminal to C-terminal. In some embodiments, a polypeptide of the multispecific binding molecule comprises the following sequences, from N-terminal to C-terminal: VH of the first binding domain, first peptide linker (e.g., a (G4S) 4 linker), VL of the first binding domain, second peptide linker (e.g., a (G4S) linker), CH2, CH3, third peptide linker (e.g., a (G4S) 4 linker), VH of the second binding domain, and CH1. In some embodiments, a polypeptide of the multispecific binding molecule comprises the following sequences: from N-terminal to C-terminal: VL of the second binding domain and CL. In some embodiments, the first binding domain comprises an anti-CD3 binding domain, e.g., an anti-CD3 scFv, e.g., comprising an anti-CD3 sequence disclosed in Table 27. In some embodiments, the second binding domain comprises a costimulatory molecule binding domain, e.g., an anti-CD2 binding domain, e.g., an anti-CD2 Fab, e.g., comprising an anti-CD2 sequence disclosed in Table 27. In some embodiments, the second binding domain comprises a costimulatory molecule binding domain, e.g., an anti-CD28 binding domain, e.g., an anti-CD28 Fab, e.g., comprising an anti-CD28 sequence disclosed in Table 27. In some embodiments, the first binding domain comprises a costimulatory molecule binding domain, e.g., an anti-CD2 or anti-CD28 binding domain, e.g., an anti-CD2 or anti-CD28 scFv, e.g., comprising an anti-CD2 or anti-CD28 sequence disclosed in Table 27. In some embodiments, the second binding domain comprises an anti-CD3 binding domain, e.g., an anti-CD3 Fab, e.g., comprising an anti-CD3 sequence disclosed in Table 27. Examples of such multispecific binding molecules are depicted as the rightmost construct in the top row of FIG. 50A; Construct 7 or Construct 8 in FIG. 51A; and Construct 7 or Construct 8 in Table 28.

In some embodiments, the first binding domain (e.g., a Fab fragment) is situated N terminal to a first Fc region. In some embodiments, the multispecific binding molecule comprises one or more of (e.g., all of) a first CH1, a first CH2, and a first CH3, e.g., in order from N-terminal to C-terminal. In some embodiments, the second binding domain (e.g., an scFv) is situated N terminal to a second Fc region, e.g., in a second polypeptide chain. In some embodiments, the multispecific binding molecule comprises, e.g., in the second polypeptide chain, one or more of (e.g., both of) a second CH2 and a second CH3, e.g., in order from N-terminal to C-terminal. In some embodiments, the multispecific binding molecule comprises a heterodimeric antibody molecule, such as for instance, wherein the first and second Fc regions comprise knob-into-hole mutations. In some embodiments, the first Fc region binds the second Fc region more strongly than the first Fc region binds another copy of the first Fc region. In some embodiments, a first polypeptide of the multispecific binding molecule comprises the following sequences, from N-terminal to C-terminal: VH of the first binding domain, a first CH1, a first CH2, and a first CH3. In some embodiments, a second polypeptide of the multispecific binding molecule comprises the following sequences, from N-terminal to C-terminal: VH of the second binding domain, a first peptide linker (e.g., a (G4S) linker), VL of the second binding domain, a second CH2, and a second CH3. In some embodiments, a third polypeptide of the multispecific binding molecule comprises the following sequences: from N-terminal to C-terminal: VL of the first binding domain and CL. In some embodiments, the second polypeptide of the multispecific binding molecule further comprises a homomultimerization domain, e.g., a Matrilin1 protein or the coiled-coil domain of cartilage oligomeric matrix protein (COMPcc), C-terminal to the second CH3, e.g., via a peptide linker (e.g., a (G4S) 4 linker, a (G4S) linker, or a (G4S) 3 linker). In some embodiments, the multispecific binding molecule comprises two, three, four, or five copies of the first binding domain and the same number of copies of the second binding domain, e.g., as depicted in FIG. 50B. In some embodiments, the first binding domain comprises a costimulatory molecule binding domain, e.g., an anti-CD2 binding domain (for example, an anti-CD2 Fab). In some embodiments, the first binding domain comprises a costimulatory molecule binding domain, e.g., an anti-CD28 binding domain (for example, an anti-CD28 Fab). In some embodiments, the second binding domain comprises an anti-CD3 binding domain, e.g., an anti-CD3 scFv. Examples of such multispecific binding molecules are depicted as the leftmost construct in the bottom row of FIG. 50A; constructs in FIG. 50B, Construct 9, Construct 10, Construct 12, Construct 13, Construct 15, and Construct 16 in FIG. 51B; and Construct 9, Construct 10, Construct 12, Construct 13, Construct 15, and Construct 16 in Table 28.

In some embodiments, a binding molecule described herein comprises a binding domain. In some embodiments the binding domain (e.g., an scFv) is situated N terminal to an Fc region. In some embodiments, the binding molecule comprises a heterodimeric antibody molecule, such as for instance, wherein the first and second Fc regions comprise knob-into-hole mutations. In some embodiments, the first Fc region binds the second Fc region more strongly than the first Fc region binds another copy of the first Fc region. In some embodiments, the binding molecule comprises one or more of (e.g., all of) a CH2 and a CH3, e.g., in order from N-terminal to C-terminal. In some embodiments, a second polypeptide of the binding molecule comprises the following sequences, from N-terminal to C-terminal: VH of the binding domain, first peptide linker (e.g., a (G4S) 4 linker), VL of the binding domain, second peptide linker (e.g., (G4S) 4 linker or (G4S) linker), CH2, and CH3. In some embodiments, the second polypeptide of the binding molecule further comprises a homomultimerization domain, e.g., a Matrilin1 protein or the coiled-coil domain of cartilage oligomeric matrix protein (COMPcc), C-terminal to the second CH3, e.g., via a peptide linker (e.g., (G4S) 4 linker, (GS4) 3 linker, or (G4S) linker). In some embodiments, the binding molecule comprises two, three, four, or five copies of the binding, e.g., as depicted in FIG. 50B. In some embodiments, the binding domain comprises an anti-CD3 binding domain, e.g., an anti-CD3 scFv. In some embodiments, a costimulatory molecule binding domain is absent. Examples of such binding molecules are depicted as the rightmost construct in the bottom row of FIG. 50A; Construct 11, Construct 14, and Construct 17 in FIG. 51B; and Construct 11, Construct 14, and Construct 17 in Table 28.

In some embodiments, the multispecific binding protein comprises an anti-CD28 binding domain, e.g., an anti-CD28 Fab, e.g., comprising an anti-CD28 (2) sequence of Table 27 (or a sequence having at least 70%, 75%, 80%, 85%, 90%, 95%, 96%, 97%, 98%, or 99% sequence identity thereto), and an anti-CD3 scFv, e.g., comprising an anti-CD3 (4) sequence of Table 27 (or a sequence having at least 70%, 75%, 80%, 85%, 90%, 95%, 96%, 97%, 98%, or 99% sequence identity thereto). In some embodiments, the multispecific binding protein comprises an Fc region, wherein the anti-CD28 Fab is fused to the Fc region, which is further fused to the anti-CD3 scFv. In some embodiments, the Fc region comprises L234A, L235A, S267K, and P329A mutations (LALASKPA), numbered according to the Eu numbering system. In some embodiments, the multispecific binding protein comprises a heavy chain comprising the amino acid sequence of SEQ ID NO: 794 or 795, or an amino acid sequence at least 70%, 75%, 80%, 85%, 90%, 95%, 96%, 97%, 98%, or 99% identical to SEQ ID NO: 794 or 795. In some embodiments, the multispecific binding protein comprises a light chain comprising the amino acid sequence of SEQ ID NO: 796 or 797, or an amino acid sequence at least 70%, 75%, 80%, 85%, 90%, 95%, 96%, 97%, 98%, or 99% identical to SEQ ID NO: 796 or 797. In some embodiments, the multispecific binding protein comprises a heavy chain comprising the amino acid sequence of SEQ ID NO: 794 or 795, or an amino acid sequence at least 70%, 75%, 80%, 85%, 90%, 95%, 96%, 97%, 98%, or 99% identical to SEQ ID NO: 794 or 795, and a light chain comprising the amino acid sequence of SEQ ID NO: 796 or 797, or an amino acid sequence at least 70%, 75%, 80%, 85%, 90%, 95%, 96%, 97%, 98%, or 99% identical to SEQ ID NO: 796 or 797. In some embodiments, the multispecific binding protein comprises a heavy chain comprising the amino acid sequence of SEQ ID NO: 794, or an amino acid sequence at least 70%, 75%, 80%, 85%, 90%, 95%, 96%, 97%, 98%, or 99% identical thereto, and a light chain comprising the amino acid sequence of SEQ ID NO: 796, or an amino acid sequence at least 70%, 75%, 80%, 85%, 90%, 95%, 96%, 97%, 98%, or 99% identical thereto. In some embodiments, the multispecific binding protein comprises a heavy chain comprising the amino acid sequence of SEQ ID NO: 794, or an amino acid sequence at least 70%, 75%, 80%, 85%, 90%, 95%, 96%, 97%, 98%, or 99% identical thereto, and a light chain comprising the amino acid sequence of SEQ ID NO: 797, or an amino acid sequence at least 70%, 75%, 80%, 85%, 90%, 95%, 96%, 97%, 98%, or 99% identical thereto. In some embodiments, the multispecific binding protein comprises a heavy chain comprising the amino acid sequence of SEQ ID NO: 795, or an amino acid sequence at least 70%, 75%, 80%, 85%, 90%, 95%, 96%, 97%, 98%, or 99% identical thereto, and a light chain comprising the amino acid sequence of SEQ ID NO: 796, or an amino acid sequence at least 70%, 75%, 80%, 85%, 90%, 95%, 96%, 97%, 98%, or 99% identical thereto. In some embodiments, the multispecific binding protein comprises a heavy chain comprising the amino acid sequence of SEQ ID NO: 795, or an amino acid sequence at least 70%, 75%, 80%, 85%, 90%, 95%, 96%, 97%, 98%, or 99% identical thereto, and a light chain comprising the amino acid sequence of SEQ ID NO: 797, or an amino acid sequence at least 70%, 75%, 80%, 85%, 90%, 95%, 96%, 97%, 98%, or 99% identical thereto.

In some embodiments, the multispecific binding protein comprises an anti-CD28 binding domain, e.g., an anti-CD28 Fab, e.g., comprising an anti-CD28 (2) sequence of Table 27 (or a sequence having at least 70%, 75%, 80%, 85%, 90%, 95%, 96%, 97%, 98%, or 99% sequence identity thereto), and an anti-CD3 scFv, e.g., comprising an anti-CD3 (2) sequence of Table 27 (or a sequence having at least 70%, 75%, 80%, 85%, 90%, 95%, 96%, 97%, 98%, or 99% sequence identity thereto). In some embodiments, the multispecific binding protein comprises an Fc region, wherein the anti-CD28 Fab is fused to the Fc region, which is further fused to the anti-CD3 scFv. In some embodiments, the Fc region comprises L234A, L235A, S267K, and P329A mutations (LALASKPA), numbered according to the Eu numbering system. In some embodiments, the multispecific binding protein comprises a heavy chain comprising the amino acid sequence of SEQ ID NO: 798 or 815, or an amino acid sequence at least 70%, 75%, 80%, 85%, 90%, 95%, 96%, 97%, 98%, or 99% identical to SEQ ID NO: 798 or 815. In some embodiments, the multispecific binding protein comprises a light chain comprising the amino acid sequence of SEQ ID NO: 799, or an amino acid sequence at least 70%, 75%, 80%, 85%, 90%, 95%, 96%, 97%, 98%, or 99% identical thereto. In some embodiments, the multispecific binding protein comprises a heavy chain comprising the amino acid sequence of SEQ ID NO: 798, or an amino acid sequence at least 70%, 75%, 80%, 85%, 90%, 95%, 96%, 97%, 98%, or 99% identical thereto, and a light chain comprising the amino acid sequence of SEQ ID NO: 799, or an amino acid sequence at least 70%, 75%, 80%, 85%, 90%, 95%, 96%, 97%, 98%, or 99% identical thereto. In some embodiments, the multispecific binding protein comprises a heavy chain comprising the amino acid sequence of SEQ ID NO: 815, or an amino acid sequence at least 70%, 75%, 80%, 85%, 90%, 95%, 96%, 97%, 98%, or 99% identical thereto, and a light chain comprising the amino acid sequence of SEQ ID NO: 799, or an amino acid sequence at least 70%, 75%, 80%, 85%, 90%, 95%, 96%, 97%, 98%, or 99% identical thereto.

In some embodiments, the multispecific binding protein comprises an anti-CD28 binding domain, e.g., an anti-CD28 Fab, e.g., comprising an anti-CD28 (1) sequence of Table 27 (or a sequence having at least 70%, 75%, 80%, 85%, 90%, 95%, 96%, 97%, 98%, or 99% sequence identity thereto) and an anti-CD3 scFv, e.g., comprising an anti-CD3 (4) sequence of Table 27 (or a sequence having at least 70%, 75%, 80%, 85%, 90%, 95%, 96%, 97%, 98%, or 99% sequence identity thereto). In some embodiments, the multispecific binding protein comprises an Fc region, wherein the anti-CD28 Fab is fused to the Fc region, which is further fused to the anti-CD3 scFv. In some embodiments, the Fc region comprises L234A, L235A, S267K, and P329A mutations (LALASKPA), numbered according to the Eu numbering system. In some embodiments, the multispecific binding protein comprises a heavy chain comprising the amino acid sequence of SEQ ID NO: 800, or an amino acid sequence at least 70%, 75%, 80%, 85%, 90%, 95%, 96%, 97%, 98%, or 99% identical thereto. In some embodiments, the multispecific binding protein comprises a light chain comprising the amino acid sequence of SEQ ID NO: 801, or an amino acid sequence at least 70%, 75%, 80%, 85%, 90%, 95%, 96%, 97%, 98%, or 99% identical thereto. In some embodiments, the multispecific binding protein comprises a heavy chain comprising the amino acid sequence of SEQ ID NO: 800, or an amino acid sequence at least 70%, 75%, 80%, 85%, 90%, 95%, 96%, 97%, 98%, or 99% identical thereto, and a light chain comprising the amino acid sequence of SEQ ID NO: 801, or an amino acid sequence at least 70%, 75%, 80%, 85%, 90%, 95%, 96%, 97%, 98%, or 99% identical thereto.

In some embodiments, the multispecific binding protein comprises an anti-CD2 binding domain, e.g., comprising an anti-CD2 (1) sequence of Table 27 (or a sequence having at least 70%, 75%, 80%, 85%, 90%, 95%, 96%, 97%, 98%, or 99% sequence identity thereto) and an anti-CD3 binding domain, comprising an anti-CD3 (1) sequence of Table 27 (or a sequence having at least 70%, 75%, 80%, 85%, 90%, 95%, 96%, 97%, 98%, or 99% sequence identity thereto). In some embodiments, the multispecific binding protein comprises an Fc region. In some embodiments, the Fc region comprises a G237A, D265A, P329A, and S267K mutation (GADAPASK), numbered according to the EU numbering system. In some embodiments, the multispecific binding protein comprises a heavy chain comprising the amino acid sequence of SEQ ID NO: 816, or an amino acid sequence at least 70%, 75%, 80%, 85%, 90%, 95%, 96%, 97%, 98%, or 99% identical thereto. In some embodiments, the multispecific binding protein comprises a light chain comprising the amino acid sequence of SEQ ID NO: 673, or an amino acid sequence at least 70%, 75%, 80%, 85%, 90%, 95%, 96%, 97%, 98%, or 99% identical thereto. In some embodiments, the multispecific binding protein comprises a heavy chain comprising the amino acid sequence of SEQ ID NO: 816, or an amino acid sequence at least 70%, 75%, 80%, 85%, 90%, 95%, 96%, 97%, 98%, or 99% identical thereto, and a light chain comprising the amino acid sequence of SEQ ID NO: 673, or an amino acid sequence at least 70%, 75%, 80%, 85%, 90%, 95%, 96%, 97%, 98%, or 99% identical thereto.

In some embodiments, the multispecific binding protein comprises an anti-CD2 binding domain, e.g., comprising an anti-CD2 (1) sequence of Table 27 (or a sequence having at least 70%, 75%, 80%, 85%, 90%, 95%, 96%, 97%, 98%, or 99% sequence identity thereto) and an anti-CD3 binding domain, e.g., comprising an anti-CD3 (1) sequence of Table 27 (or a sequence having at least 70%, 75%, 80%, 85%, 90%, 95%, 96%, 97%, 98%, or 99% sequence identity thereto). In some embodiments, the multispecific binding protein comprises an Fc region. In some embodiments, the Fc region comprises a L234A, L235A, and P329G mutation (LALAPG), numbered according to the EU numbering system. In some embodiments, the multispecific binding protein comprises a heavy chain comprising the amino acid sequence of SEQ ID NO: 817, or an amino acid sequence at least 70%, 75%, 80%, 85%, 90%, 95%, 96%, 97%, 98%, or 99% identical thereto. In some embodiments, the multispecific binding protein comprises a light chain comprising the amino acid sequence of SEQ ID NO: 673, or an amino acid sequence at least 70%, 75%, 80%, 85%, 90%, 95%, 96%, 97%, 98%, or 99% identical thereto. In some embodiments, the multispecific binding protein comprises a heavy chain comprising the amino acid sequence of SEQ ID NO: 817, or an amino acid sequence at least 70%, 75%, 80%, 85%, 90%, 95%, 96%, 97%, 98%, or 99% identical thereto, and a light chain comprising the amino acid sequence of SEQ ID NO: 673, or an amino acid sequence at least 70%, 75%, 80%, 85%, 90%, 95%, 96%, 97%, 98%, or 99% identical thereto.

It is understood that in many of the embodiments herein, a multispecific binding molecule comprises two or more polypeptide chains that are covalently linked to each other, e.g., via a disulfide bridge. However, in some embodiments, the two or more polypeptide chains of the multispecific binding molecule may be noncovalently bound to each other.

It is also understood that a Fab fragment may be present as part of a larger protein, for instance, a Fab fragment may be fused with CH2 and CH3 and thus be part of full length antibody.

The multispecific binding molecule comprising an agent that stimulates a CD3/TCR complex and an agent that stimulates a costimulatory molecule and/or growth factor receptor disclosed herein is contemplated for use in the manufacturing embodiments disclosed herein, e.g., traditional manufacture or activated rapid manufacture.

In some embodiments, the multispecific binding molecule is a multispecific binding molecule described, e.g., in WO2021173985, the contents of which are hereby incorporated by reference in their entirety.

### Fc Variants

In some embodiments, a multispecific binding molecule described herein comprises an Fc region, e.g., as described herein. In some embodiments, the Fc region is a wild type Fc region, e.g., a wild type human Fc region. In some embodiments, the Fc region comprises a variant, e.g., an Fc region comprising an addition, substitution, or deletion of at least one amino acid residue in the Fc region which results in, e.g., reduced or ablated affinity for at least one Fc receptor.

In some embodiments, the Fc region of an antibody interacts with a number of receptors or ligands including Fc Receptors (e.g., FcγRI, FcγRIIA, FcγRIIIA), the complement protein CIq, and other molecules such as proteins A and G. These interactions promote a variety of effector functions and downstream signaling events including: antibody dependent cell-mediated cytotoxicity (ADCC), Antibody-dependent cellular phagocytosis (ADCP) and complement dependent cytotoxicity (CDC).

In some embodiments, a multispecific binding molecule described herein comprising a variant Fc region has reduced, e.g., ablated, affinity for an Fc receptor, e.g., an Fc receptor described herein. In some embodiments, the reduced affinity is compared to an otherwise similar antibody with a wild type Fc region.

In some embodiments, a multispecific binding molecule described herein comprising a variant Fc region has one or more of the following properties: (1) reduced effector function (e.g., reduced ADCC, ADCP and/or CDC); (2) reduced binding to one or more Fc receptors; and/or (3) reduced binding to C1q complement. In some embodiments, the reduction in any one, or all of properties (1)-(3) is compared to an otherwise similar antibody with a wildtype Fc region.

Exemplary Fc region variants are provided in Table 34 and also disclosed in Saunders O, (2019) Frontiers in Immunology; vol 10, article 1296, the entire contents of which is hereby incorporated by reference.

In some embodiments, a multispecific binding molecule described herein comprises any one or all, or any combination of Fc region variants, e.g., mutations, disclosed in Table 34. In some embodiments, the Fc region of a multispecific binding molecule described herein is silenced. In some embodiments, the Fc region of a multispecific binding protein described herein is silenced by a combination of amino acid substitutions selected from the group consisting of LALA, DAPA, DANAPA, LALADANAPS, LALAGA, LALASKPA, DAPASK, GADAPA, GADAPASK, LALAPG, and LALAPA (numbered according to the Eu numbering system).

In some embodiments, a multispecific binding molecule described herein comprises any one or all, or any combination of a mutant comprising a L234, e.g., L234A and/or L235, e.g., L234A, mutation (LALA) in the IgG1 Fc amino acid sequence, numbered according to the Eu numbering system; D265, e.g., D265A and/or P329, e.g., P329A (DAPA); N297, e.g., N297A; DANAPA (D265A, N297A, and P329A), numbered according to the Eu numbering system; and/or L234, e.g. L234A, L235, e.g., L235A, D265, e.g., D265A, N297, e.g., N297A, and P331, e.g., P331S (LALADANAPS), wherein the amino acid residues are numbered according to the EU numbering system. In some embodiments, a multispecific binding molecule described herein comprises any one or all, or any combination of a mutant comprising a L234, e.g. L234A, L235, e.g. L235A, and G237, e.g. G237A, mutation (LALAGA) in the IgG1 Fc amino acid sequence, numbered according to the Eu numbering system; L234, e.g. L234A, L235, e.g. L235A, S267, e.g. S267K, and P239, e.g. P329A (LALASKPA), numbered according to the Eu numbering system; D265, e.g. D265A, P239, e.g. P329A, and S267, e.g. S267K (DAPASK), numbered according to the Eu numbering system; G237, e.g. G237A, D265, e.g. D265A, and P329, e.g. P329A (GADAPA), numbered according to the Eu numbering system; G236, e.g. G237A, D265, e.g. D265A, P329, e.g. P329A, and S267, e.g. S267K (GADAPASK), numbered according to the Eu numbering system; L234, e.g. L234A, L 235, e.g. L235A, and P329, e.g. P329G (LALAPG), numbered according to the Eu numbering system; and/or L234, e.g. L234A, L235, e.g. L235A, and P329, e.g. P329A (LALAPA), wherein the amino acid residues are numbered according to the EU numbering system.

In some embodiments, the Fc region of a multispecific binding protein described herein comprises a mutation that results in reduced binding to an Fc receptor or reduced ADCC, ADCP, or CDC activity, e.g., an Fc region comprising: a D265 (e.g., D265A), N297 (e.g., N297A), and P329 (e.g., P329A) mutation (DANAPA), numbered according to the Eu numbering system; an L234 (e.g., L234A), L235 (e.g., L235A), and G237 (G237A) mutation (LALAGA), numbered according to the Eu numbering system; an L234 (L234A), L235 (e.g., L235A), S267 (e.g., S267K), and P329 (e.g., P329A) mutation (LALASKPA), numbered according to the Eu numbering system; a D265 (e.g., D265A), P329 (e.g., P329A), and S267 (e.g., S267K) mutation (DAPASK), numbered according to the Eu numbering system; a G237 (e.g., G237A), a D265 (e.g., D265A), and P329 (P329A) mutation (GADAPA), numbered according to the Eu numbering system; a G237 (e.g., G237A), D265 (e.g., D265A), P329 (e.g., P329A), and S267 (e.g., S267K) mutation (GADAPASK), numbered according to the Eu numbering system; an L234 (e.g., L234A), L235 (e.g., L235A), and P329 (e.g., P329G) mutation (LALAPG), numbered according to the Eu numbering system; or an L234 (e.g., L234A), L235 (e.g., L235A), and P329 (e.g., P329A) mutation (LALAPA), numbered according to the Eu numbering system.

In some embodiments, the Fc region comprises a mutation at one, two, three or all of positions L234 (e.g. L234A), L235 (e.g. L235A), S267 (e.g. S267K), and P239 (e.g. P329A), numbered according to the Eu numbering system. In some embodiments, the Fc region comprises a mutation at L234 (e.g. L234A), L235 (e.g. L235A), S267 (e.g. S267K), and P239 (e.g. P329A) (LALASKPA), numbered according to the EU numbering system. In some embodiments, the Fc region comprises a L234A, L235A, S267K, and P329A mutation (LALASKPA), numbered according to the EU numbering system.

TABLE 34
Exemplary Fc modifications

| Modification or mutation | Altered effector function |
|---|---|
| Leu235Glu | ADCC; |
| Leu234Ala/Leu235Ala (LALA) | ADCC; ADCP; CDC |
| Ser228Pro/Leu235Glu | |
| Leu234Ala/Leu235Ala/Pro329Gly | ADCP |
| Pro331Ser/Leu234Glu/Leu235Phe | CDC |
| Asp265Ala | ADCC, ADCP |
| Gly237Ala | ADCP |
| Glu318Ala | ADCP |
| Glu233Pro | |
| Gly236Arg/Leu328Arg | ADCC |
| His268Gln/Val309Leu/Ala330Ser/Pro331Ser | ADCC; ADCP; CDC |
| Val234Ala/Gly237Ala/Pro238Ser/ | ADCC; ADCP; CDC |
| His268Ala/Val309Leu/Ala330Ser/Pro331Ser | |
| Leu234Ala/L235Ala/Gly237Ala/P238Ser/ | ADCC; CDC |
| His268Ala/Ala330Ser/Pro331Ser | |
| Ala330Leu | CDC |
| Asp270Ala | CDC |
| Lys322Ala | CDC |
| Pro329Ala | CDC |
| Pro331Ala | CDC |
| Val264Ala | CDC |
| High mannose glycosylation | CDC |
| Phe241 Ala | CDC |
| Asn297Ala or Gly or Gln | ADCC; ADCP; CDC |
| S228P/Phe234Ala/Leu235Ala | ADCC; CDC |

Further non-limiting exemplary Fc modifications include LALAGA (L234A, L235A, and G237A), LALASKPA (L234A, L235A, S267K, and P329A), DAPASK (D265A, P329A, and S267K), GADAPA (G237A, D265A, and P329A), GADAPASK (G237A, D265A, P329A, and S267K), LALAPG (L234A, L235A, and P329G), and LALAPA (L234A, L235A, and P329A), wherein the amino acid residues are numbered according to the EU numbering system.

Non-limiting exemplary Fc regions featuring these and other silencing modifications disclosed herein are provided in Table 28 above. Additional non-limiting exemplary Fc regions featuring these and other silencing modifications disclosed herein are provided in Table 35 below.

TABLE 35
Additional exemplary silenced Fc sequences

| SEQ ID | | |
|---|---|---|
| No: | Chain | Amino acid sequence |
| SEQ ID | Human IgG1 Fc variant | APEAAGGPSVFLFPPKPKDTLMISRTPEVTCVVV |
| NO: 806 | L234A/L235A/P329A | DVSHEDPEVKFNWYVDGVEVHNAKTKPREEQY |
| | (LALAPA) | NSTYRVVSVLTVLHQDWLNGKEYKCKVSNKAL |
| | Amino acid sequence | AAPIEKTISKAKGQPREPQVYTLPPSRDELTKNQV |
| | | SLTCLVKGFYPSDIAVEWESNGQPENNYKTTPPV |
| | | LDSDGSFFLYSKLTVDKSRWQQGNVFSCSVMHE |
| | | ALHNHYTQKSLSLSPGK |
| SEQ ID | Human IgG1 Fc variant | APEAAGAPSVFLFPPKPKDTLMISRTPEVTCVVV |
| NO: 807 | L234A/L235A/G237A | DVSHEDPEVKFNWYVDGVEVHNAKTKPREEQY |
| | (LALAGA) | NSTYRVVSVLTVLHQDWLNGKEYKCKVSNKAL |
| | Amino acid sequence | PAPIEKTISKAKGQPREPQVYTLPPSRDELTKNQV |
| | | SLTCLVKGFYPSDIAVEWESNGQPENNYKTTPPV |
| | | LDSDGSFFLYSKLTVDKSRWQQGNVFSCSVMHE |
| | | ALHNHYTQKSLSLSPGK |
| SEQ ID | Human IgG1 Fc variant | APEAAGGPSVFLFPPKPKDTLMISRTPEVTCVVV |
| NO: 808 | L234A/L235A/P329G | DVSHEDPEVKFNWYVDGVEVHNAKTKPREEQY |
| | (LALAPG) | NSTYRVVSVLTVLHQDWLNGKEYKCKVSNKAL |
| | Amino acid sequence | GAPIEKTISKAKGQPREPQVYTLPPSRDELTKNQV |
| | | SLTCLVKGFYPSDIAVEWESNGQPENNYKTTPPV |
| | | LDSDGSFFLYSKLTVDKSRWQQGNVFSCSVMHE |
| | | ALHNHYTQKSLSLSPGK |
| SEQ ID | Human IgG1 Fc variant | APELLGGPSVFLFPPKPKDTLMISRTPEVTCVVVA |
| NO: 809 | D265A/P329A | VSHEDPEVKFNWYVDGVEVHNAKTKPREEQYN |
| | (DAPA) | STYRVVSVLTVLHQDWLNGKEYKCKVSNKALA |
| | Amino acid sequence | APIEKTISKAKGQPREPQVYTLPPSRDELTKNQVS |
| | | LTCLVKGFYPSDIAVEWESNGQPENNYKTTPPVL |
| | | DSDGSFFLYSKLTVDKSRWQQGNVFSCSVMHEA |
| | | LHNHYTQKSLSLSPGK |
| SEQ ID | Human IgG1 Fc variant | APEAAGGPSVFLFPPKPKDTLMISRTPEVTCVVV |
| NO: 810 | L234A/L235A/S267K/ | DVKHEDPEVKFNWYVDGVEVHNAKTKPREEQY |
| | P329A (LALASKPA) | NSTYRVVSVLTVLHQDWLNGKEYKCKVSNKAL |
| | Amino acid sequence | AAPIEKTISKAKGQPREPQVYTLPPSRDELTKNQV |
| | | SLTCLVKGFYPSDIAVEWESNGQPENNYKTTPPV |
| | | LDSDGSFFLYSKLTVDKSRWQQGNVFSCSVMHE |
| | | ALHNHYTQKSLSLSPGK |
| SEQ ID | Human IgG1 Fc variant | APELLGGPSVFLFPPKPKDTLMISRTPEVTCVVVA |
| NO: 811 | D265A/P329A/S267K | VKHEDPEVKFNWYVDGVEVHNAKTKPREEQYN |
| | (DAPASK) | STYRVVSVLTVLHQDWLNGKEYKCKVSNKALA |
| | Amino acid sequence | APIEKTISKAKGQPREPQVYTLPPSRDELTKNQVS |
| | | LTCLVKGFYPSDIAVEWESNGQPENNYKTTPPVL |
| | | DSDGSFFLYSKLTVDKSRWQQGNVFSCSVMHEA |
| | | LHNHYTQKSLSLSPGK |
| SEQ ID | Human IgG1 Fc variant | APELLGAPSVFLFPPKPKDTLMISRTPEVTCVVVA |
| NO: 812 | G237A/D265A/P329A | VSHEDPEVKFNWYVDGVEVHNAKTKPREEQYN |
| | (GADAPA) | STYRVVSVLTVLHQDWLNGKEYKCKVSNKALA |
| | Amino acid sequence | APIEKTISKAKGQPREPQVYTLPPSRDELTKNQVS |
| | | LTCLVKGFYPSDIAVEWESNGQPENNYKTTPPVL |
| | | DSDGSFFLYSKLTVDKSRWQQGNVFSCSVMHEA |
| | | LHNHYTQKSLSLSPGK |
| SEQ ID | Human IgG1 Fc variant | APELLGAPSVFLFPPKPKDTLMISRTPEVTCVVVA |
| NO: 813 | G237A/D265A/P329A/ | VKHEDPEVKFNWYVDGVEVHNAKTKPREEQYN |
| | S267K (GADAPASK) | STYRVVSVLTVLHQDWLNGKEYKCKVSNKALA |
| | Amino acid sequence | APIEKTISKAKGQPREPQVYTLPPSRDELTKNQVS |
| | | LTCLVKGFYPSDIAVEWESNGQPENNYKTTPPVL |
| | | DSDGSFFLYSKLTVDKSRWQQGNVFSCSVMHEA |
| | | LHNHYTQKSLSLSPGK |
| SEQ ID | Human IgG1 Fc variant | APELLGGPSVFLFPPKPKDTLMISRTPEVTCVVVA |
| NO: 814 | D265A/N297A/P329A | VSHEDPEVKFNWYVDGVEVHNAKTKPREEQYA |
| | (DANAPA) | STYRVVSVLTVLHQDWLNGKEYKCKVSNKALA |
| | Amino acid sequence | APIEKTISKAKGQPREPQVYTLPPSRDELTKNQVS |
| | | LTCLVKGFYPSDIAVEWESNGQPENNYKTTPPVL |
| | | DSDGSFFLYSKLTVDKSRWQQGNVFSCSVMHEA |
| | | LHNHYTQKSLSLSPGK |

### Population of CAR-Expressing Cells Manufactured by the Processes Disclosed Herein

In some embodiments, the disclosure features an immune effector cell (for example, T cell or NK cell), for example, made by any of the manufacturing methods described herein, engineered to express a CAR, wherein the engineered immune effector cell exhibits an antitumor property. In some embodiments, the CAR comprises an antigen binding domain, a transmembrane domain, and an intracellular signaling domain. An exemplary antigen is a cancer associated antigen described herein. In some embodiments, the cell (for example, T cell or NK cell) is transformed with the CAR and the CAR is expressed on the cell surface. In some embodiments, the cell (for example, T cell or NK cell) is transduced with a viral vector encoding the CAR. In some embodiments, the viral vector is a retroviral vector. In some embodiments, the viral vector is a lentiviral vector. In some such embodiments, the cell may stably express the CAR. In some embodiments, the cell (for example, T cell or NK cell) is transfected with a nucleic acid, for example, mRNA, cDNA, or DNA, encoding a CAR. In some such embodiments, the cell may transiently express the CAR.

In some embodiments, provided herein is a population of cells (for example, immune effector cells, for example, T cells or NK cells) made by any of the manufacturing processes described herein (for example, the cytokine process, or the activation process described herein), engineered to express a CAR.

In some embodiments, the percentage of naïve cells, for example, naïve T cells, for example, CD45RA+CD45RO− CCR7+ T cells, in the population of cells at the end of the manufacturing process (for example, at the end of the cytokine process or the activation process described herein) (1) is the same as, (2) differs, for example, by no more than 4, 5, 6, 7, 8, 9, 10, 11, 12, 13, 14, or 15%, from, or (3) is increased, for example, by at least 5, 6, 7, 8, 9, 10, 11, 12, 13, 14, 15, 16, 17, 18, 19, 20, 21, 22, 23, 24, or 25%, as compared to, the percentage of naïve cells, for example, naïve T cells, for example, CD45RA+CD45RO− CCR7+ cells, in the population of cells at the beginning of the manufacturing process (for example, at the beginning of the cytokine process or the activation process described herein). In some embodiments, the population of cells at the end of the manufacturing process (for example, at the end of the cytokine process or the activation process described herein) shows a higher percentage of naïve cells, for example, naïve T cells, for example, CD45RA+CD45RO− CCR7+ T cells (for example, at least 5, 6, 7, 8, 9, 10, 11, 12, 13, 14, 15, 16, 17, 18, 19, 20, 25, 30, 35, 40, 45, or 50% higher), compared with cells made by an otherwise similar method which lasts, for example, more than 26 hours (for example, which lasts more than 5, 6, 7, 8, 9, 10, 11, or 12 days) or which involves expanding the population of cells in vitro for, for example, more than 3 days (for example, expanding the population of cells in vitro for 3, 4, 5, 6, 7, 8, 9, 10, 11, 12, 13, 14, or 15 days).

In some embodiments, the percentage of naïve cells, for example, naïve T cells, for example, CD45RA+CD45RO− CCR7+ T cells, in the population of cells at the end of the manufacturing process (for example, at the end of the cytokine process or the activation process described herein) is not less than 20, 25, 30, 35, 40, 45, 50, 55, or 60%.

In some embodiments, the percentage of central memory cells, for example, central memory T cells, for example, CD95+ central memory T cells, in the population of cells at the end of the manufacturing process (for example, at the end of the cytokine process or the activation process described herein) (1) is the same as, (2) differs, for example, by no more than 4, 5, 6, 7, 8, 9, 10, 11, 12, 13, 14, or 15% from, or (3) is decreased, for example, by at least 5, 6, 7, 8, 9, 10, 11, 12, 13, 14, 15, 16, 17, 18, 19, 20, 21, 22, 23, 24, or 25%, as compared to, the percentage of central memory cells, for example, central memory T cells, for example, CD95+ central memory T cells, in the population of cells at the beginning of the manufacturing process (for example, at the beginning of the cytokine process or the activation process described herein). In some embodiments, the population of cells at the end of the manufacturing process (for example, at the end of the cytokine process or the activation process described herein) shows a lower percentage of central memory cells, for example, central memory T cells, for example, CD95+ central memory T cells (for example, at least 5, 6, 7, 8, 9, 10, 11, 12, 13, 14, 15, 16, 17, 18, 19, 20, 25, 30, 35, 40, 45, or 50% lower), compared with cells made by an otherwise similar method which lasts, for example, more than 26 hours (for example, which lasts more than 5, 6, 7, 8, 9, 10, 11, or 12 days) or which involves expanding the population of cells in vitro for, for example, more than 3 days (for example, expanding the population of cells in vitro for 3, 4, 5, 6, 7, 8, 9, 10, 11, 12, 13, 14, or 15 days).

In some embodiments, the percentage of central memory cells, for example, central memory T cells, for example, CD95+ central memory T cells, in the population of cells at the end of the manufacturing process (for example, at the end of the cytokine process or the activation process described herein) is no more than 40, 45, 50, 55, 60, 65, 70, 75, or 80%.

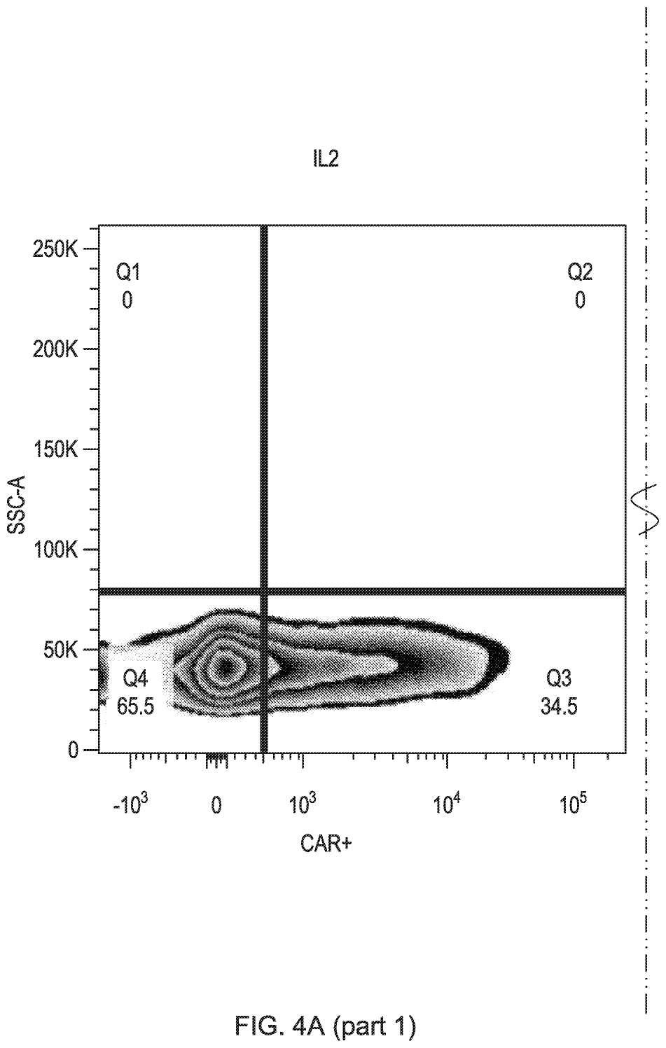

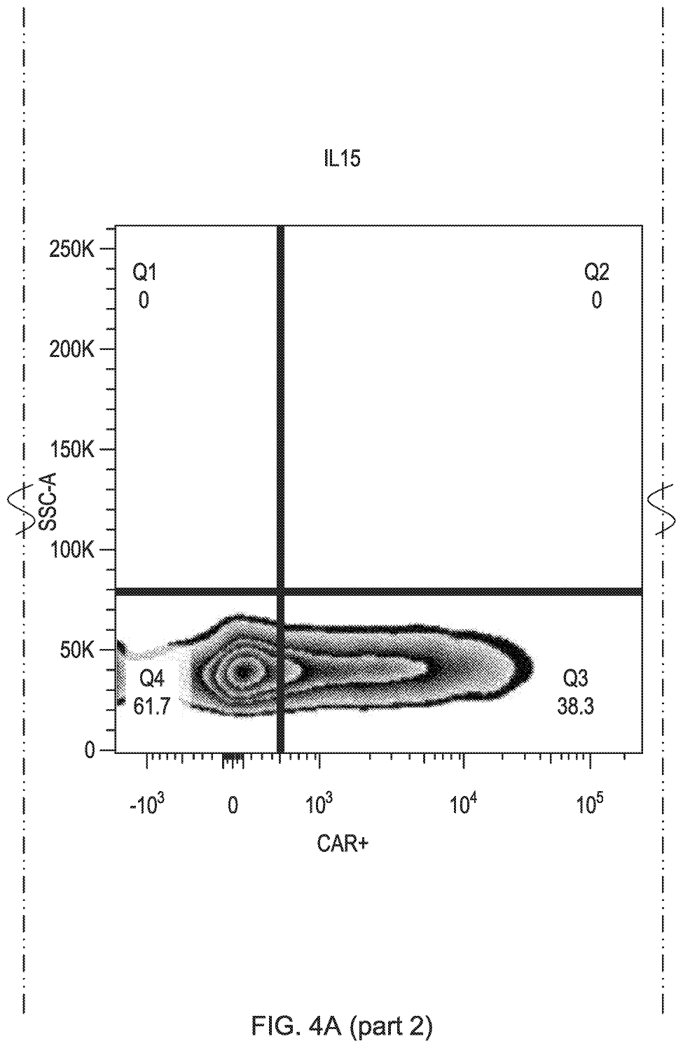

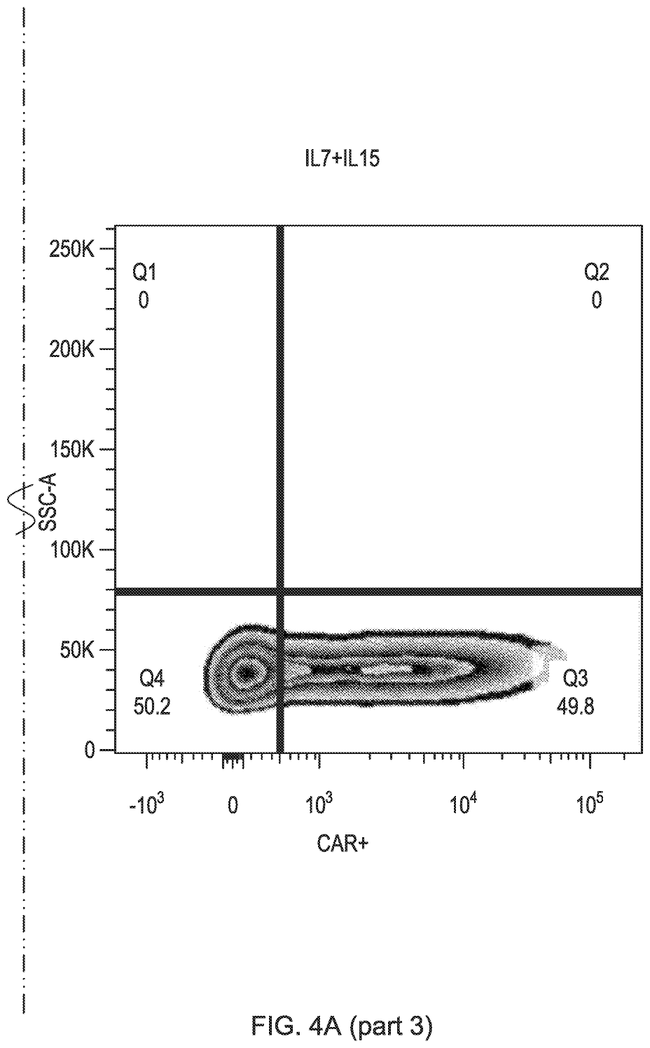

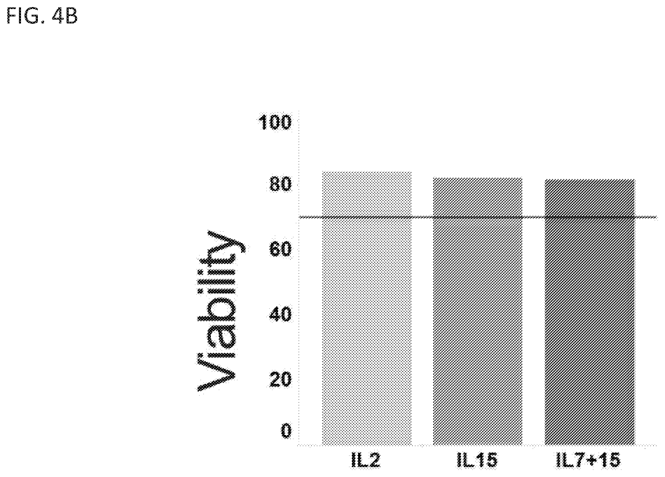

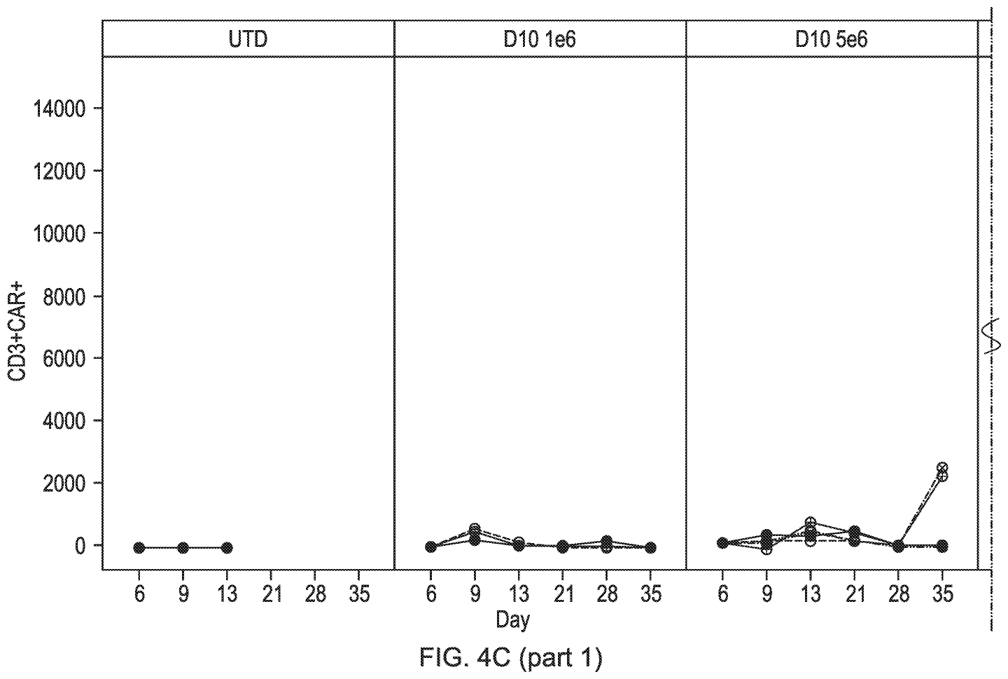

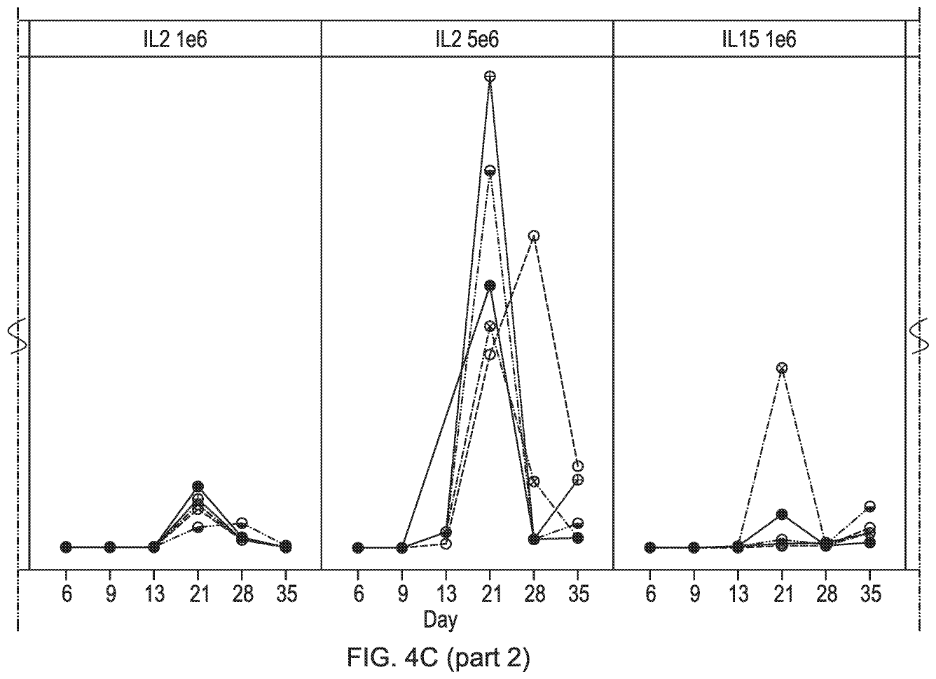

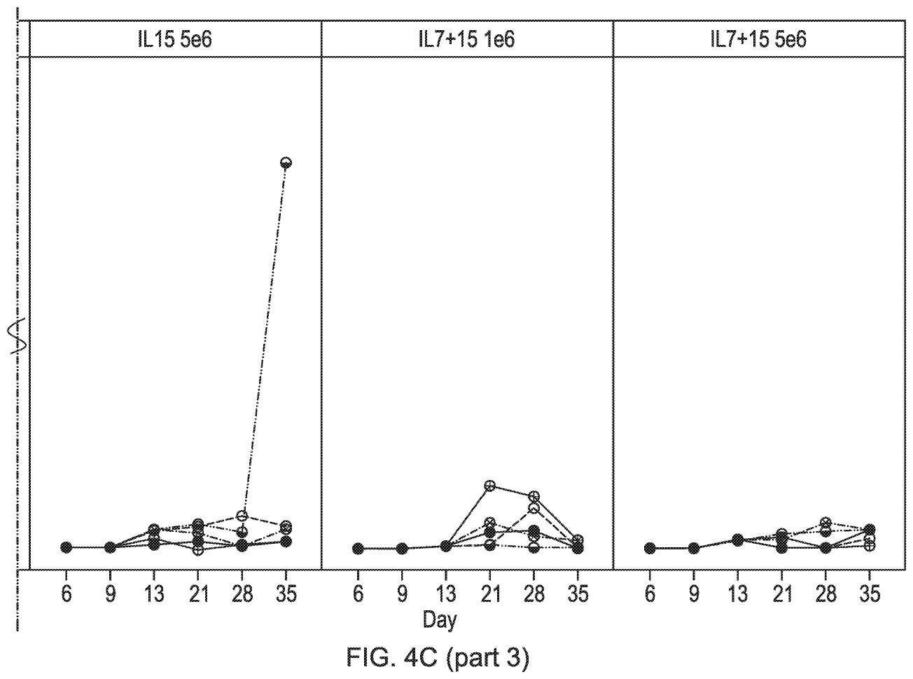

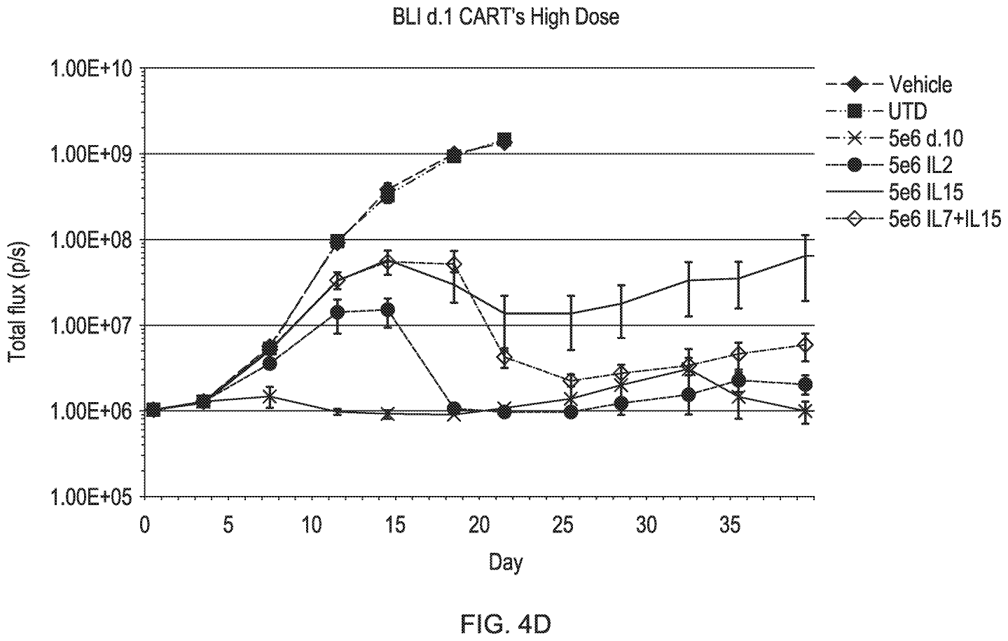

In some embodiments, the population of cells at the end of the manufacturing process (for example, at the end of the cytokine process or the activation process described herein) after being administered in vivo, persists longer or expands at a higher level (for example, at least 20, 25, 30, 35, 40, 45, 50, 55, 60, 65, 70, 75, 80, 85, or 90% higher) (for example, as assessed using methods described in Example 1 with respect to FIG. 4C), compared with cells made by an otherwise similar method which lasts, for example, more than 26 hours (for example, which lasts more than 5, 6, 7, 8, 9, 10, 11, or 12 days) or which involves expanding the population of cells in vitro for, for example, more than 3 days (for example, expanding the population of cells in vitro for 3, 4, 5, 6, 7, 8, 9, 10, 11, 12, 13, 14, or 15 days).

In some embodiments, the population of cells has been enriched for IL6R-expressing cells (for example, cells that are positive for IL6Rα and/or IL6Rβ) prior to the beginning of the manufacturing process (for example, prior to the beginning of the cytokine process or the activation process described herein). In some embodiments, the population of cells comprises, for example, no less than 30, 35, 40, 45, 50, 55, 60, 65, 70, 75, or 80% of IL6R-expressing cells (for example, cells that are positive for IL6Rα and/or IL6Rβ) at the beginning of the manufacturing process (for example, at the beginning of the cytokine process or the activation process described herein).

### Pharmaceutical Composition

Furthermore, the present disclosure provides CAR-expressing cell compositions and their use in medicaments or methods for treating, among other diseases, cancer or any malignancy or autoimmune diseases involving cells or tissues which express a tumor antigen as described herein. In some embodiments, provided herein are pharmaceutical compositions comprising a CAR-expressing cell, for example, a plurality of CAR-expressing cells, made by a manufacturing process described herein (for example, the cytokine process, or the activation process described herein), in combination with one or more pharmaceutically or physiologically acceptable carriers, diluents or excipients.

### Chimeric Antigen Receptor (CAR)

The present invention provides immune effector cells (for example, T cells or NK cells) that are engineered to contain one or more CARs that direct the immune effector cells to cancer. This is achieved through an antigen binding domain on the CAR that is specific for a cancer associated antigen. There are two classes of cancer associated antigens (tumor antigens) that can be targeted by the CARs described herein: (1) cancer associated antigens that are expressed on the surface of cancer cells; and (2) cancer associated antigens that themselves are intracellular, however, fragments (peptides) of such antigens are presented on the surface of the cancer cells by MHC (major histocompatibility complex).

Accordingly, an immune effector cell, for example, obtained by a method described herein, can be engineered to contain a CAR that targets one of the following cancer associated antigens (tumor antigens): CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, VEGFR2, Lewis Y, CD24, PDGFR-beta, PRSS21, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRC5D, CXORF61, CD97, CD179a, ALK, Plysialic acid, PLAC1, GloboH, NY-BR-1, UPK2, HAVCR1, ADRB3, PANX3, GPR20, LY6K, OR51E2, TARP, WT1, NY-ESO-1, LAGE-la, legumain, HPV E6,E7, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, and mut hsp70-2.

Sequences of non-limiting examples of various components that can be part of a CAR molecule described herein are listed in Table 1, where “aa” stands for amino acids, and “na” stands for nucleic acids that encode the corresponding peptide.

TABLE 1
Sequences of various components of CAR

| SEQ ID NO | Description | Sequence |
|---|---|---|
| SEQ ID NO: | EF-1α promoter | CGTGAGGCTCCGGTGCCCGTCAGTGGGCAGAGCGCACAT |
| 11 | (na) | CGCCCACAGTCCCCGAGAAGTTGGGGGGAGGGGTCGGC |
| | | AATTGAACCGGTGCCTAGAGAAGGTGGCGCGGGGTAAA |
| | | CTGGGAAAGTGATGTCGTGTACTGGCTCCGCCTTTTTCCC |
| | | GAGGGTGGGGGAGAACCGTATATAAGTGCAGTAGTCGC |
| | | CGTGAACGTTCTTTTTCGCAACGGGTTTGCCGCCAGAAC |
| | | ACAGGTAAGTGCCGTGTGTGGTTCCCGCGGGCCTGGCCT |
| | | CTTTACGGGTTATGGCCCTTGCGTGCCTTGAATTACTTCC |
| | | ACCTGGCTGCAGTACGTGATTCTTGATCCCGAGCTTCGG |
| | | GTTGGAAGTGGGTGGGAGAGTTCGAGGCCTTGCGCTTAA |
| | | GGAGCCCCTTCGCCTCGTGCTTGAGTTGAGGCCTGGCCT |
| | | GGGCGCTGGGGCCGCCGCGTGCGAATCTGGTGGCACCTT |
| | | CGCGCCTGTCTCGCTGCTTTCGATAAGTCTCTAGCCATTT |
| | | AAAATTTTTGATGACCTGCTGCGACGCTTTTTTTCTGGCA |
| | | AGATAGTCTTGTAAATGCGGGCCAAGATCTGCACACTGG |
| | | TATTTCGGTTTTTGGGGCCGCGGGCGGCGACGGGGCCCG |
| | | TGCGTCCCAGCGCACATGTTCGGCGAGGCGGGGCCTGCG |
| | | AGCGCGGCCACCGAGAATCGGACGGGGGTAGTCTCAAG |
| | | CTGGCCGGCCTGCTCTGGTGCCTGGCCTCGCGCCGCCGT |
| | | GTATCGCCCCGCCCTGGGCGGCAAGGCTGGCCCGGTCGG |
| | | CACCAGTTGCGTGAGCGGAAAGATGGCCGCTTCCCGGCC |
| | | CTGCTGCAGGGAGCTCAAAATGGAGGACGCGGCGCTCG |
| | | GGAGAGCGGGCGGGTGAGTCACCCACACAAAGGAAAAG |
| | | GGCCTTTCCGTCCTCAGCCGTCGCTTCATGTGACTCCACG |
| | | GAGTACCGGGCGCCGTCCAGGCACCTCGATTAGTTCTCG |
| | | AGCTTTTGGAGTACGTCGTCTTTAGGTTGGGGGGAGGGG |
| | | TTTTATGCGATGGAGTTTCCCCACACTGAGTGGGTGGAG |
| | | ACTGAAGTTAGGCCAGCTTGGCACTTGATGTAATTCTCC |
| | | TTGGAATTTGCCCTTTTTGAGTTTGGATCTTGGTTCATTC |
| | | TCAAGCCTCAGACAGTGGTTCAAAGTTTTTTTCTTCCATT |
| | | TCAGGTGTCGTGA |
| SEQ ID NO: | Leader (aa) | MALPVTALLLPLALLLHAARP |
| 1 | | |
| SEQ ID NO: | Leader (na) | ATGGCCCTGCCTGTGACAGCCCTGCTGCTGCCTCTGGCTC |
| 12 | | TGCTGCTGCATGCCGCTAGACCC |
| SEQ ID NO: | Leader (na) | ATGGCCCTCCCTGTCACCGCCCTGCTGCTTCCGCTGGCTC |
| 199 | | TTCTGCTCCACGCCGCTCGGCCC |
| SEQ ID NO: | CD8 hinge (aa) | TTTPAPRPPTPAPTIASQPLSLRPEACRPAAGGAVHTRGLDF |
| 2 | | ACD |
| SEQ ID NO: | CD8 hinge (na) | ACCACGACGCCAGCGCCGCGACCACCAACACCGGCGCC |
| 13 | | CACCATCGCGTCGCAGCCCCTGTCCCTGCGCCCAGAGGC |
| | | GTGCCGGCCAGCGGCGGGGGGCGCAGTGCACACGAGGG |
| | | GGCTGGACTTCGCCTGTGAT |
| SEQ ID NO: | Ig4 hinge (aa) | ESKYGPPCPPCPAPEFLGGPSVFLFPPKPKDTLMISRTPEVTC |
| 3 | | VVVDVSQEDPEVQFNWYVDGVEVHNAKTKPREEQFNSTY |
| | | RVVSVLTVLHQDWLNGKEYKCKVSNKGLPSSIEKTISKAK |
| | | GQPREPQVYTLPPSQEEMTKNQVSLTCLVKGFYPSDIAVEW |
| | | ESNGQPENNYKTTPPVLDSDGSFFLYSRLTVDKSRWQEGN |
| | | VFSCSVMHEALHNHYTQKSLSLSLGKM |
| SEQ ID NO: | Ig4 hinge (na) | GAGAGCAAGTACGGCCCTCCCTGCCCCCCTTGCCCTGCC |
| 14 | | CCCGAGTTCCTGGGCGGACCCAGCGTGTTCCTGTTCCCC |
| | | CCCAAGCCCAAGGACACCCTGATGATCAGCCGGACCCCC |
| | | GAGGTGACCTGTGTGGTGGTGGACGTGTCCCAGGAGGAC |
| | | CCCGAGGTCCAGTTCAACTGGTACGTGGACGGCGTGGAG |
| | | GTGCACAACGCCAAGACCAAGCCCCGGGAGGAGCAGTT |
| | | CAATAGCACCTACCGGGTGGTGTCCGTGCTGACCGTGCT |
| | | GCACCAGGACTGGCTGAACGGCAAGGAATACAAGTGTA |
| | | AGGTGTCCAACAAGGGCCTGCCCAGCAGCATCGAGAAA |
| | | ACCATCAGCAAGGCCAAGGGCCAGCCTCGGGAGCCCCA |
| | | GGTGTACACCCTGCCCCCTAGCCAAGAGGAGATGACCAA |
| | | GAACCAGGTGTCCCTGACCTGCCTGGTGAAGGGCTTCTA |
| | | CCCCAGCGACATCGCCGTGGAGTGGGAGAGCAACGGCC |
| | | AGCCCGAGAACAACTACAAGACCACCCCCCCTGTGCTGG |
| | | ACAGCGACGGCAGCTTCTTCCTGTACAGCCGGCTGACCG |
| | | TGGACAAGAGCCGGTGGCAGGAGGGCAACGTCTTTAGC |
| | | TGCTCCGTGATGCACGAGGCCCTGCACAACCACTACACC |
| | | CAGAAGAGCCTGAGCCTGTCCCTGGGCAAGATG |
| SEQ ID NO: | IgD hinge (aa) | RWPESPKAQASSVPTAQPQAEGSLAKATTAPATTRNTGRG |
| 4 | | GEEKKKEKEKEEQEERETKTPECPSHTQPLGVYLLTPAVQD |
| | | LWLRDKATFTCFVVGSDLKDAHLTWEVAGKVPTGGVEEG |
| | | LLERHSNGSQSQHSRLTLPRSLWNAGTSVTCTLNHPSLPPQ |
| | | RLMALREPAAQAPVKLSLNLLASSDPPEAASWLLCEVSGFS |
| | | PPNILLMWLEDQREVNTSGFAPARPPPQPGSTTFWAWSVLR |
| | | VPAPPSPQPATYTCVVSHEDSRTLLNASRSLEVSYVTDH |
| SEQ ID NO: | IgD hinge (na) | AGGTGGCCCGAAAGTCCCAAGGCCCAGGCATCTAGTGTT |
| 15 | | CCTACTGCACAGCCCCAGGCAGAAGGCAGCCTAGCCAA |
| | | AGCTACTACTGCACCTGCCACTACGCGCAATACTGGCCG |
| | | TGGCGGGGAGGAGAAGAAAAAGGAGAAAGAGAAAGAA |
| | | GAACAGGAAGAGAGGGAGACCAAGACCCCTGAATGTCC |
| | | ATCCCATACCCAGCCGCTGGGCGTCTATCTCTTGACTCCC |
| | | GCAGTACAGGACTTGTGGCTTAGAGATAAGGCCACCTTT |
| | | ACATGTTTCGTCGTGGGCTCTGACCTGAAGGATGCCCAT |
| | | TTGACTTGGGAGGTTGCCGGAAAGGTACCCACAGGGGG |
| | | GGTTGAGGAAGGGTTGCTGGAGCGCCATTCCAATGGCTC |
| | | TCAGAGCCAGCACTCAAGACTCACCCTTCCGAGATCCCT |
| | | GTGGAACGCCGGGACCTCTGTCACATGTACTCTAAATCA |
| | | TCCTAGCCTGCCCCCACAGCGTCTGATGGCCCTTAGAGA |
| | | GCCAGCCGCCCAGGCACCAGTTAAGCTTAGCCTGAATCT |
| | | GCTCGCCAGTAGTGATCCCCCAGAGGCCGCCAGCTGGCT |
| | | CTTATGCGAAGTGTCCGGCTTTAGCCCGCCCAACATCTT |
| | | GCTCATGTGGCTGGAGGACCAGCGAGAAGTGAACACCA |
| | | GCGGCTTCGCTCCAGCCCGGCCCCCACCCCAGCCGGGTT |
| | | CTACCACATTCTGGGCCTGGAGTGTCTTAAGGGTCCCAG |
| | | CACCACCTAGCCCCCAGCCAGCCACATACACCTGTGTTG |
| | | TGTCCCATGAAGATAGCAGGACCCTGCTAAATGCTTCTA |
| | | GGAGTCTGGAGGTTTCCTACGTGACTGACCATT |
| SEQ ID NO: | CD8 | IYIWAPLAGTCGVLLLSLVITLYC |
| 6 | Transmembrane | |
| | (aa) | |
| SEQ ID NO: | CD8 | ATCTACATCTGGGCGCCCTTGGCCGGGACTTGTGGGGTC |
| 17 | Transmembrane | CTTCTCCTGTCACTGGTTATCACCCTTTACTGC |
| | (na) | |
| SEQ ID NO: | 4-1BB intracellular | KRGRKKLLYIFKQPFMRPVQTTQEEDGCSCRFPEEEEGGCE |
| 7 | domain (aa) | L |
| SEQ ID NO: | 4-1BB intracellular | AAACGGGGCAGAAAGAAACTCCTGTATATATTCAAACA |
| 18 | domain (na) | ACCATTTATGAGACCAGTACAAACTACTCAAGAGGAAG |
| | | ATGGCTGTAGCTGCCGATTTCCAGAAGAAGAAGAAGGA |
| | | GGATGTGAACTG |
| SEQ ID NO: | CD27 (aa) | QRRKYRSNKGESPVEPAEPCRYSCPREEEGSTIPIQEDYRKP |
| 8 | | EPACSP |
| SEQ ID NO: | CD27 (na) | AGGAGTAAGAGGAGCAGGCTCCTGCACAGTGACTACAT |
| 19 | | GAACATGACTCCCCGCCGCCCCGGGCCCACCCGCAAGCA |
| | | TTACCAGCCCTATGCCCCACCACGCGACTTCGCAGCCTA |
| | | TCGCTCC |
| SEQ ID NO: | CD3-zeta (aa) | RVKFSRSADAPAYKQGQNQLYNELNLGRREEYDVLDKRR |
| 9 | (Q/K mutant) | GRDPEMGGKPRRKNPQEGLYNELQKDKMAEAYSEIGMKG |
| | | ERRRGKGHDGLYQGLSTATKDTYDALHMQALPPR |
| SEQ ID NO: | CD3-zeta (na) | AGAGTGAAGTTCAGCAGGAGCGCAGACGCCCCCGCGTA |
| 20 | (Q/K mutant) | CAAGCAGGGCCAGAACCAGCTCTATAACGAGCTCAATCT |
| | | AGGACGAAGAGAGGAGTACGATGTTTTGGACAAGAGAC |
| | | GTGGCCGGGACCCTGAGATGGGGGGAAAGCCGAGAAGG |
| | | AAGAACCCTCAGGAAGGCCTGTACAATGAACTGCAGAA |
| | | AGATAAGATGGCGGAGGCCTACAGTGAGATTGGGATGA |
| | | AAGGCGAGCGCCGGAGGGGCAAGGGGCACGATGGCCTT |
| | | TACCAGGGTCTCAGTACAGCCACCAAGGACACCTACGAC |
| | | GCCCTTCACATGCAGGCCCTGCCCCCTCGC |
| SEQ ID NO: | CD3-zeta (aa) | RVKFSRSADAPAYQQGQNQLYNELNLGRREEYDVLDKRR |
| 10 | (NCBI Reference | GRDPEMGGKPRRKNPQEGLYNELQKDKMAEAYSEIGMKG |
| | Sequence | ERRRGKGHDGLYQGLSTATKDTYDALHMQALPPR |
| | NM_000734.3) | |
| SEQ ID NO: | CD3-zeta (na) | AGAGTGAAGTTCAGCAGGAGCGCAGACGCCCCCGCGTA |
| 21 | (NCBI Reference | CCAGCAGGGCCAGAACCAGCTCTATAACGAGCTCAATCT |
| | Sequence | AGGACGAAGAGAGGAGTACGATGTTTTGGACAAGAGAC |
| | NM_000734.3) | GTGGCCGGGACCCTGAGATGGGGGGAAAGCCGAGAAGG |
| | | AAGAACCCTCAGGAAGGCCTGTACAATGAACTGCAGAA |
| | | AGATAAGATGGCGGAGGCCTACAGTGAGATTGGGATGA |
| | | AAGGCGAGCGCCGGAGGGGCAAGGGGCACGATGGCCTT |
| | | TACCAGGGTCTCAGTACAGCCACCAAGGACACCTACGAC |
| | | GCCCTTCACATGCAGGCCCTGCCCCCTCGC |
| SEQ ID NO: | CD28 Intracellular | RSKRSRLLHSDYMNMTPRRPGPTRKHYQPYAPPRDFAAYR |
| 36 | domain (amino acid | S |
| | sequence) | |
| SEQ ID NO: | CD28 Intracellular | AGGAGTAAGAGGAGCAGGCTCCTGCACAGTGACTACAT |
| 37 | domain (nucleotide | GAACATGACTCCCCGCCGCCCCGGGCCCACCCGCAAGCA |
| | sequence) | TTACCAGCCCTATGCCCCACCACGCGACTTCGCAGCCTA |
| | | TCGCTCC |
| SEQ ID NO: | ICOS Intracellular | TKKKYSSSVHDPNGEYMFMRAVNTAKKSR |
| 38 | domain (amino acid | LTDVTL |
| | sequence) | |
| SEQ ID NO: | ICOS Intracellular | ACAAAAAAGAAGTATTCATCCAGTGTGCACGACCCTAAC |
| 39 | domain (nucleotide | GGTGAATACATGTTCATGAGAGCAGTGAACACAGCCAA |
| | sequence) | AAAATCCAGACTCACAGATGTGACCCTA |
| SEQ ID NO: | GS hinge/linker | GGGGSGGGGS |
| 5 | (aa) | |
| SEQ ID NO: | GS hinge/linker | GGTGGCGGAGGTTCTGGAGGTGGAGGTTCC |
| 16 | (na) | |
| SEQ ID NO: | GS hinge/linker | GGTGGCGGAGGTTCTGGAGGTGGGGGTTCC |
| 40 | (na) | |
| SEQ ID NO: | linker | GGGGS |
| 25 | | |
| SEQ ID NO: | linker | (Gly-Gly-Gly-Gly-Ser)n, where n = 1-6, for example, |
| 26 | | GGGGSGGGGS GGGGSGGGGS GGGGSGGGGS |
| SEQ ID NO: | linker | GGGGSGGGGSGGGGGGGGS |
| 27 | | |
| SEQ ID NO: | linker | GGGGSGGGGSGGGGS |
| 28 | | |
| SEQ ID NO: | linker | GGGS |
| 29 | | |
| SEQ ID NO: | linker | (Gly-Gly-Gly-Ser)n where n is a positive integer equal to or |
| 41 | | greater than 1 |
| SEQ ID NO: | linker | (Gly-Gly-Gly-Ser)n, where n = 1-10, for example, |
| 42 | | GGGSGGGSGG GSGGGSGGGS GGGSGGGSGG |
| | | GSGGGSGGGS |
| SEQ ID NO: | linker | GSTSGSGKPGSGEGSTKG |
| 43 | | |
| SEQ ID NO: | poly(A) | (A)₅₀₀₀ |
| 30 | | This sequence may encompass 50-5000 adenines. |
| SEQ ID NO: | polyT | (T)₁₀₀ |
| 31 | | |
| SEQ ID NO: | polyT | (T)₅₀₀₀ |
| 32 | | This sequence may encompass 50-5000 thymines. |
| SEQ ID NO: | poly(A) | (A)₅₀₀₀ |
| 33 | | This sequence may encompass 100-5000 adenines. |
| SEQ ID NO: | poly(A) | (A)₄₀₀ |
| 34 | | This sequence may encompass 100-400 adenines. |
| SEQ ID NO: | poly(A) | (A)₂₀₀₀ |
| 35 | | This sequence may encompass 50-2000 adenines. |
| SEQ ID NO: | PD1 CAR (aa) | pgwfldspdrpwnpptfspallvvtegdnatftcsfsntsesfvlnwyrmspsnqtdklaaf |
| 22 | | pedrsqpgqdcrfrvtqlpngrdfhmsvvrarrndsgtylcgaislapkagikeslraelrvt |
| | | erraevptahpspsprpagqfqtlvtttpaprpptpaptiasqplslrpeacrpaaggavhtr |
| | | gldfacdiyiwaplagtcgvlllslvitlyckrgrkkllyifkqpfmrpvqttqeedgescrf |
| | | peeeeggcelrvkfsrsadapaykqgqnqlynelnlgrreeydvldkrrgrdpemggkprrk |
| | | npqeglynelqkdkmaeayseigmkgerrrgkghdglyqglstatkdtydalhmqalpp |
| | | r |
| SEQ ID NO: | PD-1 CAR (na) | atggccctccctgtcactgccctgcttctccccctegcactcctgctccacgccgctagacca |
| 23 | (PD1 ECD | cccggatggtttctggactctccggatcgcccgtggaatcccccaaccttctcaccggcactc |
| | underlined) | ttggttgtgactgagggcgataatgcgaccttcacgtgctcgttctccaacacctccgaatca |
| | | ttcgtgctgaactggtaccgcatgagcccgtcaaaccagaccgacaagctcgccgcgtttccg |
| | | gaagatcggtcgcaaccgggacaggattgtcggttccgcgtgactcaactgccgaatggcaga |
| | | gacttccacatgagcgtggtccgcgctaggcgaaacgactccgggacctacctgtgcggagc |
| | | catctcgctggcgcctaaggcccaaatcaaagagagcttgagggccgaactgagagtgacc |
| | | gagcgcagagctgaggtgccaactgcacatccatccccatcgcctoggcctgcggggcagt |
| | | ttcagaccctggtcacgaccactccggcgccgcgcccaccgactceggccccaactatogc |
| | | gagccagcccctgtcgctgaggccggaagcatgccgccctgccgccggaggtgctgtgcat |
| | | acccggggattggacttcgcatgcgacatctacatttgggctcctctcgccggaacttgtggc |
| | | gtgctccttctgtccctggtcatcaccctgtactgcaagcggggtcggaaaaagcttctgtac |
| | | attttcaagcagcccttcatgaggcccgtgcaaaccacccaggaggaggacggttgctcctgc |
| | | cggttccccgaagaggaagaaggaggttgcgagctgcgcgtgaagttctcccggagcgccgac |
| | | gcccccgcctataagcagggccagaaccagctgtacaacgaactgaacctgggacggcgg |
| | | gaagagtacgatgtgctggacaagcggcgcggccgggaccccgaaatggggggaagcc |
| | | tagaagaaagaaccctcaggaaggcctgtataacgagctgcagaaggacaagatggccga |
| | | ggcctactccgaaattgggatgaagggagagcggcggaggggaaaggggcacgacggcc |
| | | tgtaccaaggactgtccaccgccaccaaggacacatacgatgccctgcacatgcaggccctt |
| | | ccccctcgc |
| SEQ ID NO: | PD-1 CAR (aa) | Malpvtalllplalllhaarppgwfldspdrpwnpptfspallvvtegdnatftcsfsntses |
| 24 | with signal | fvlnwyrmspsnqtdklaafpedrsqpgqdcrfrvtqlpngrdfhmsvvrarrndsgtylc |
| | (PD1 ECD | gaislapkaqikeslraelrvterraevptahpspsprpagqfqtlvtttpaprpptpaptia |
| | underlined) | sqplslrpeacrpaaggavhtrgldfacdiyiwaplagtcgvlllslvitlyckrgrkkllyi |
| | | fkqpfmrpvqttqeedgescrfpeeeeggcelrvkfsrsadapaykqgqnqlynelnlgrree |
| | | ydvldkrrgrdpemggkprrknpqeglynelqkdkmaeayseigmkgerrrgkghdglyq |
| | | glstatkdtydalhmqalppr |

### Bispecific CARs

In some embodiments a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some embodiments the first and second epitopes are on the same antigen, for example, the same protein (or subunit of a multimeric protein). In some embodiments the first and second epitopes overlap. In some embodiments the first and second epitopes do not overlap. In some embodiments the first and second epitopes are on different antigens, for example, different proteins (or different subunits of a multimeric protein). In some embodiments a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In some embodiments a bispecific antibody molecule comprises a half antibody having binding specificity for a first epitope and a half antibody having binding specificity for a second epitope. In some embodiments a bispecific antibody molecule comprises a half antibody, or fragment thereof, having binding specificity for a first epitope and a half antibody, or fragment thereof, having binding specificity for a second epitope. In some embodiments a bispecific antibody molecule comprises a scFv, or fragment thereof, have binding specificity for a first epitope and a scFv, or fragment thereof, have binding specificity for a second epitope.

In certain embodiments, the antibody molecule is a multi-specific (for example, a bispecific or a trispecific) antibody molecule. Protocols for generating bispecific or heterodimeric antibody molecules, and various configurations for bispecific antibody molecules, are described in, for example, paragraphs 455-458 of WO2015/142675, filed Mar. 13, 2015, which is incorporated by reference in its entirety.

In some embodiments, the bispecific antibody molecule is characterized by a first immunoglobulin variable domain sequence, for example, a scFv, which has binding specificity for CD19, for example, comprises a scFv as described herein, or comprises the light chain CDRs and/or heavy chain CDRs from a scFv described herein, and a second immunoglobulin variable domain sequence that has binding specificity for a second epitope on a different antigen.

### Chimeric TCR

In some embodiments, the antibodies and antibody fragments of the present invention (for example, CD19 antibodies and fragments) can be grafted to one or more constant domain of a T cell receptor (“TCR”) chain, for example, a TCR alpha or TCR beta chain, to create a chimeric TCR. Without being bound by theory, it is believed that chimeric TCRs will signal through the TCR complex upon antigen binding. For example, an scFv as disclosed herein, can be grafted to the constant domain, for example, at least a portion of the extracellular constant domain, the transmembrane domain and the cytoplasmic domain, of a TCR chain, for example, the TCR alpha chain and/or the TCR beta chain. As another example, an antibody fragment, for example a VL domain as described herein, can be grafted to the constant domain of a TCR alpha chain, and an antibody fragment, for example a VH domain as described herein, can be grafted to the constant domain of a TCR beta chain (or alternatively, a VL domain may be grafted to the constant domain of the TCR beta chain and a VH domain may be grafted to a TCR alpha chain). As another example, the CDRs of an antibody or antibody fragment may be grafted into a TCR alpha and/or beta chain to create a chimeric TCR. For example, the LCDRs disclosed herein may be grafted into the variable domain of a TCR alpha chain and the HCDRs disclosed herein may be grafted to the variable domain of a TCR beta chain, or vice versa. Such chimeric TCRs may be produced, for example, by methods known in the art (For example, Willemsen R A et al, Gene Therapy 2000; 7:1369-1377; Zhang T et al, Cancer Gene Ther 2004; 11:487-496; Aggen et al, Gene Ther. 2012 April; 19 (4): 365-74).

### Non-Antibody Scaffolds

In embodiments, the antigen binding domain comprises a non-antibody scaffold, for example, a fibronectin, ankyrin, domain antibody, lipocalin, small modular immuno-pharmaceutical, maxybody, Protein A, or affilin. The non-antibody scaffold has the ability to bind to target antigen on a cell. In embodiments, the antigen binding domain is a polypeptide or fragment thereof of a naturally occurring protein expressed on a cell. In some embodiments, the antigen binding domain comprises a non-antibody scaffold. A wide variety of non-antibody scaffolds can be employed so long as the resulting polypeptide includes at least one binding region which specifically binds to the target antigen on a target cell.

Non-antibody scaffolds include: fibronectin (Novartis, MA), ankyrin (Molecular Partners AG, Zurich, Switzerland), domain antibodies (Domantis, Ltd., Cambridge, MA, and Ablynx nv, Zwijnaarde, Belgium), lipocalin (Pieris Proteolab AG, Freising, Germany), small modular immuno-pharmaceuticals (Trubion Pharmaceuticals Inc., Seattle, WA), maxybodies (Avidia, Inc., Mountain View, CA), Protein A (Affibody AG, Sweden), and affilin (gamma-crystallin or ubiquitin) (Scil Proteins GmbH, Halle, Germany).

In some embodiments the antigen binding domain comprises the extracellular domain, or a counter-ligand binding fragment thereof, of molecule that binds a counterligand on the surface of a target cell.

The immune effector cells can comprise a recombinant DNA construct comprising sequences encoding a CAR, wherein the CAR comprises an antigen binding domain (for example, antibody or antibody fragment, TCR or TCR fragment) that binds specifically to a tumor antigen, for example, a tumor antigen described herein, and an intracellular signaling domain. The intracellular signaling domain can comprise a costimulatory signaling domain and/or a primary signaling domain, for example, a zeta chain. As described elsewhere, the methods described herein can include transducing a cell, for example, from the population of T regulatory-depleted cells, with a nucleic acid encoding a CAR, for example, a CAR described herein.

In some embodiments, a CAR comprises a scFv domain, wherein the scFv may be preceded by an optional leader sequence such as provided in SEQ ID NO: 1, and followed by an optional hinge sequence such as provided in SEQ ID NO:2 or SEQ ID NO:36 or SEQ ID NO: 38, a transmembrane region such as provided in SEQ ID NO:6, an intracellular signaling domain that includes SEQ ID NO:7 or SEQ ID NO: 16 and a CD3 zeta sequence that includes SEQ ID NO:9 or SEQ ID NO:10, for example, wherein the domains are contiguous with and in the same reading frame to form a single fusion protein.

In some embodiments, an exemplary CAR constructs comprise an optional leader sequence (for example, a leader sequence described herein), an extracellular antigen binding domain (for example, an antigen binding domain described herein), a hinge (for example, a hinge region described herein), a transmembrane domain (for example, a transmembrane domain described herein), and an intracellular stimulatory domain (for example, an intracellular stimulatory domain described herein). In some embodiments, an exemplary CAR construct comprises an optional leader sequence (for example, a leader sequence described herein), an extracellular antigen binding domain (for example, an antigen binding domain described herein), a hinge (for example, a hinge region described herein), a transmembrane domain (for example, a transmembrane domain described herein), an intracellular costimulatory signaling domain (for example, a costimulatory signaling domain described herein) and/or an intracellular primary signaling domain (for example, a primary signaling domain described herein).

An exemplary leader sequence is provided as SEQ ID NO: 1. An exemplary hinge/spacer sequence is provided as SEQ ID NO: 2 or SEQ ID NO:36 or SEQ ID NO:38. An exemplary transmembrane domain sequence is provided as SEQ ID NO:6. An exemplary sequence of the intracellular signaling domain of the 4-1BB protein is provided as SEQ ID NO: 7. An exemplary sequence of the intracellular signaling domain of CD27 is provided as SEQ ID NO: 16. An exemplary CD3zeta domain sequence is provided as SEQ ID NO: 9 or SEQ ID NO: 10.

In some embodiments, the immune effector cell comprises a recombinant nucleic acid construct comprising a nucleic acid molecule encoding a CAR, wherein the nucleic acid molecule comprises a nucleic acid sequence encoding an antigen binding domain, wherein the sequence is contiguous with and in the same reading frame as the nucleic acid sequence encoding an intracellular signaling domain. An exemplary intracellular signaling domain that can be used in the CAR includes, but is not limited to, one or more intracellular signaling domains of, for example, CD3-zeta, CD28, CD27, 4-1BB, and the like. In some instances, the CAR can comprise any combination of CD3-zeta, CD28, 4-1BB, and the like.

The nucleic acid sequences coding for the desired molecules can be obtained using recombinant methods known in the art, such as, for example by screening libraries from cells expressing the nucleic acid molecule, by deriving the nucleic acid molecule from a vector known to include the same, or by isolating directly from cells and tissues containing the same, using standard techniques. Alternatively, the nucleic acid of interest can be produced synthetically, rather than cloned.

Nucleic acids encoding a CAR can be introduced into the immune effector cells using, for example, a retroviral or lentiviral vector construct.

Nucleic acids encoding a CAR can also be introduced into the immune effector cell using, for example, an RNA construct that can be directly transfected into a cell. A method for generating mRNA for use in transfection involves in vitro transcription (IVT) of a template with specially designed primers, followed by poly(A) addition, to produce a construct containing 3′ and 5′ untranslated sequence (“UTR”) (for example, a 3′ and/or 5′ UTR described herein), a 5′ cap (for example, a 5′ cap described herein) and/or Internal Ribosome Entry Site (IRES) (for example, an IRES described herein), the nucleic acid to be expressed, and a poly(A) tail, typically 50-2000 bases in length (for example, described in the Examples, for example, SEQ ID NO:35). RNA so produced can efficiently transfect different kinds of cells. In some embodiments, the template includes sequences for the CAR. In some embodiments, an RNA CAR vector is transduced into a cell, for example, a T cell by electroporation.

### Antigen Binding Domain

In some embodiments, a plurality of the immune effector cells, for example, the population of T regulatory-depleted cells, include a nucleic acid encoding a CAR that comprises a target-specific binding element otherwise referred to as an antigen binding domain. The choice of binding element depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain in a CAR described herein include those associated with viral, bacterial and parasitic infections, autoimmune disease and cancer cells.

In some embodiments, the portion of the CAR comprising the antigen binding domain comprises an antigen binding domain that targets a tumor antigen, for example, a tumor antigen described herein.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, for example, single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment.

### CD19 CAR

In some embodiments, the CAR-expressing cell described herein is a CD19 CAR-expressing cell (for example, a cell expressing a CAR that binds to human CD19).

In some embodiments, the antigen binding domain of the CD19 CAR has the same or a similar binding specificity as the FMC63 scFv fragment described in Nicholson et al. Mol. Immun. 34 (16-17): 1157-1165 (1997). In some embodiments, the antigen binding domain of the CD19 CAR includes the scFv fragment described in Nicholson et al. Mol. Immun. 34 (16-17): 1157-1165 (1997).

In some embodiments, the CD19 CAR includes an antigen binding domain (for example, a humanized antigen binding domain) according to Table 3 of WO2014/153270, incorporated herein by reference. WO2014/153270 also describes methods of assaying the binding and efficacy of various CAR constructs.

In some embodiments, the parental murine scFv sequence is the CAR19 construct provided in PCT publication WO2012/079000 (incorporated herein by reference). In some embodiments, the anti-CD19 binding domain is a scFv described in WO2012/079000.

In some embodiments, the CAR molecule comprises the fusion polypeptide sequence provided as SEQ ID NO: 12 in PCT publication WO2012/079000, which provides an scFv fragment of murine origin that specifically binds to human CD19.

In some embodiments, the CD19 CAR comprises an amino acid sequence provided as SEQ ID NO: 12 in PCT publication WO2012/079000.

In some embodiments, the amino acid sequence is:
Diqmtqttsslsaslgdrvtiscrasqdiskylnwyqqkpdgtvklliyhtsrlhsgvpsrfsgsgsgtdysltisnleqediat yfcqqgntlpytfgggtkleitggggsggggsggggsevklqesgpglvapsqslsvtctvsgvslpdygvswirqpprkglewlgv iwgsettyynsalksrltiikdnsksqvflkmnslqtddtaiyycakhyyyggsyamdywgqgtsvtvsstttpaprpptpaptiasq plslrpeacrpaaggavhtrgldfacdiyiwaplagtcgvlllslvitlyckrgrkkllyifkqpfmrpvqttqeedgcscrfpeeeeggc elrvkfsrsadapaykqgqnqlynelnlgrreeydvldkrrgrdpemggkprrknpqeglynelqkdkmaeayseigmkgerrrg kghdglyqglstatkdtydalhmqalppr (SEQ ID NO: 292), or a sequence substantially homologous thereto.

In some embodiments, the CD19 CAR has the USAN designation TISAGENLECLEUCEL-T. In embodiments, CTL019 is made by a gene modification of T cells is mediated by stable insertion via transduction with a self-inactivating, replication deficient Lentiviral (LV) vector containing the CTL019 transgene under the control of the EF-1 alpha promoter. CTL019 can be a mixture of transgene positive and negative T cells that are delivered to the subject on the basis of percent transgene positive T cells.

In other embodiments, the CD19 CAR comprises an antigen binding domain (for example, a humanized antigen binding domain) according to Table 3 of WO2014/153270, incorporated herein by reference.

Humanization of murine CD19 antibody is desired for the clinical setting, where the mouse-specific residues may induce a human-anti-mouse antigen (HAMA) response in patients who receive CART19 treatment, i.e., treatment with T cells transduced with the CAR19 construct. The production, characterization, and efficacy of humanized CD19 CAR sequences is described in International Application WO2014/153270 which is herein incorporated by reference in its entirety, including Examples 1-5 (p. 115-159).

In some embodiments, the CAR molecule is a humanized CD19 CAR comprising the amino acid sequence of:

(SEQ ID NO: 293)
EIVMTQSPATLSLSPGERATLSCRASQDISKYLNWYQQKPGQAPR
LLIYHTSRLHSGIPARFSGSGSGTDYTLTISSLQPEDFAVYFCQQ
GNTLPYTFGQGTKLEIKGGGGSGGGGSGGGGSQVQLQESGPGLVK
PSETLSLTCTVSGVSLPDYGVSWIRQPPGKGLEWIGVIWGSETTY
YQSSLKSRVTISKDNSKNQVSLKLSSVTAADTAVYYCAKHYYYGG
SYAMDYWGQGTLVTVSS

In some embodiments, the CAR molecule is a humanized CD19 CAR comprising the amino acid sequence of:

(SEQ ID NO: 294)
EIVMTQSPATLSLSPGERATLSCRASQDISKYLNWYQQKPGQAPR
LLIYHTSRLHSGIPARFSGSGSGTDYTLTISSLQPEDFAVYFCQQ
GNTLPYTFGQGTKLEIKGGGGSGGGGSGGGGSQVQLQESGPGLVK
PSETLSLTCTVSGVSLPDYGVSWIRQPPGKGLEWIGVIWGSETTY
YQSSLKSRVTISKDNSKNQVSLKLSSVTAADTAVYYCAKHYYYGG
SYAMDYWGQGTLVTVSSTTTPAPRPPTPAPTIASQPLSLRPEACR
PAAGGAVHTRGLDFACDIYIWAPLAGTCGVLLLSLVITLYCKRGR
KKLLYIFKQPFMRPVQTTQEEDGCSCRFPEEEEGGCELRVKFSRS
ADAPAYKQGQNQLYNELNLGRREEYDVLDKRRGRDPEMGGKPRRK
NPQEGLYNELQKDKMAEAYSEIGMKGERRRGKGHDGLYQGLSTAT
KDTYDALHMQALPPR

Any known CD19 CAR, for example, the CD19 antigen binding domain of any known CD19 CAR, in the art can be used in accordance with the present disclosure. For example, LG-740; CD19 CAR described in the U.S. Pat. Nos. 8,399,645; 7,446,190; Xu et al., Leuk Lymphoma. 2013 54 (2): 255-260 (2012); Cruz et al., Blood 122 (17): 2965-2973 (2013); Brentjens et al., Blood, 118 (18): 4817-4828 (2011); Kochenderfer et al., Blood 116 (20): 4099-102 (2010); Kochenderfer et al., Blood 122 (25): 4129-39 (2013); and 16th Annu Meet Am Soc Gen Cell Ther (ASGCT) (May 15-18, Salt Lake City) 2013, Abst 10.

Exemplary CD19 CARs include CD19 CARs described herein or an anti-CD19 CAR described in Xu et al. Blood 123.24 (2014): 3750-9; Kochenderfer et al. Blood 122.25 (2013): 4129-39, Cruz et al. Blood 122.17 (2013): 2965-73, NCT00586391, NCT01087294, NCT02456350, NCT00840853, NCT02659943, NCT02650999, NCT02640209, NCT01747486, NCT02546739, NCT02656147, NCT02772198, NCT00709033, NCT02081937, NCT00924326, NCT02735083, NCT02794246, NCT02746952, NCT01593696, NCT02134262, NCT01853631, NCT02443831, NCT02277522, NCT02348216, NCT02614066, NCT02030834, NCT02624258, NCT02625480, NCT02030847, NCT02644655, NCT02349698, NCT02813837, NCT02050347, NCT01683279, NCT02529813, NCT02537977, NCT02799550, NCT02672501, NCT02819583, NCT02028455, NCT01840566, NCT01318317, NCT01864889, NCT02706405, NCT01475058, NCT01430390, NCT02146924, NCT02051257, NCT02431988, NCT01815749, NCT02153580, NCT01865617, NCT02208362, NCT02685670, NCT02535364, NCT02631044, NCT02728882, NCT02735291, NCT01860937, NCT02822326, NCT02737085, NCT02465983, NCT02132624, NCT02782351, NCT01493453, NCT02652910, NCT02247609, NCT01029366, NCT01626495, NCT02721407, NCT01044069, NCT00422383, NCT01680991, NCT02794961, or NCT02456207, each of which is incorporated herein by reference in its entirety.

In some embodiments, CD19 CARs comprise a sequence, for example, a CDR, VH, VL, scFv, or full-CAR sequence, disclosed in Table 2, or a sequence having at least 80%, 85%, 90%, 95%, or 99% identity thereto.

TABLE 2
Amino acid sequences of exemplary
anti-CD19 molecules

| SEQ ID | | |
|---|---|---|
| NO | Region | Sequence |
| CTL019 | | |
| 295 | HCDR1 | DYGVS |
| | (Kabat) | |
| 296 | HCDR2 | VIWGSETTYYNSALKS |
| | (Kabat) | |
| 297 | HCDR3 | HYYYGGSYAMDY |
| | (Kabat) | |
| 298 | LCDR1 | RASQDISKYLN |
| | (Kabat) | |
| 299 | LCDR2 | HTSRLHS |
| | (Kabat) | |
| 300 | LCDR3 | QQGNTLPYT |
| | (Kabat) | |
| 301 | CTL019 | MALPVTALLLPLALLLHAAR |
| | Full amino | PDIQMTQTTSSLSASLGDRV |
| | acid | TISCRASQDISKYLNWYQQK |
| | sequence | PDGTVKLLIYHTSRLHSGVP |
| | | SRFSGSGSGTDYSLTISNLE |
| | | QEDIATYFCQQGNTLPYTFG |
| | | GGTKLEITGGGGSGGGGSGG |
| | | GGSEVKLQESGPGLVAPSQS |
| | | LSVTCTVSGVSLPDYGVSWI |
| | | RQPPRKGLEWLGVIWGSETT |
| | | YYNSALKSRLTIIKDNSKSQ |
| | | VFLKMNSLQTDDTAIYYCAK |
| | | HYYYGGSYAMDYWGQGTSVT |
| | | VSSTTTPAPRPPTPAPTIAS |
| | | QPLSLRPEACRPAAGGAVHT |
| | | RGLDFACDIYIWAPLAGTCG |
| | | VLLLSLVITLYCKRGRKKLL |
| | | YIFKQPFMRPVQTTQEEDGC |
| | | SCRFPEEEEGGCELRVKFSR |
| | | SADAPAYKQGQNQLYNELNL |
| | | GRREEYDVLDKRRGRDPEMG |
| | | GKPRRKNPQEGLYNELQKDK |
| | | MAEAYSEIGMKGERRRGKGH |
| | | DGLYQGLSTATKDTYDALHM |
| | | QALPPR |
| 302 | CTL019 | ATGGCCTTACCAGTGACCGC |
| | Full | CTTGCTCCTGCCGCTGGCCT |
| | nucleotide | TGCTGCTCCACGCCGCCAGG |
| | sequence | CCGGACATCCAGATGACACA |
| | | GACTACATCCTCCCTGTCTG |
| | | CCTCTCTGGGAGACAGAGTC |
| | | ACCATCAGTTGCAGGGCAAG |
| | | TCAGGACATTAGTAAATATT |
| | | TAAATTGGTATCAGCAGAAA |
| | | CCAGATGGAACTGTTAAACT |
| | | CCTGATCTACCATACATCAA |
| | | GATTACACTCAGGAGTCCCA |
| | | TCAAGGTTCAGTGGCAGTGG |
| | | GTCTGGAACAGATTATTCTC |
| | | TCACCATTAGCAACCTGGAG |
| | | CAAGAAGATATTGCCACTTA |
| | | CTTTTGCCAACAGGGTAATA |
| | | CGCTTCCGTACACGTTCGGA |
| | | GGGGGGACCAAGCTGGAGAT |
| | | CACAGGTGGCGGTGGCTCGG |
| | | GCGGTGGTGGGTCGGGTGGC |
| | | GGCGGATCTGAGGTGAAACT |
| | | GCAGGAGTCAGGACCTGGCC |
| | | TGGTGGCGCCCTCACAGAGC |
| | | CTGTCCGTCACATGCACTGT |
| | | CTCAGGGGTCTCATTACCCG |
| | | ACTATGGTGTAAGCTGGATT |
| | | CGCCAGCCTCCACGAAAGGG |
| | | TCTGGAGTGGCTGGGAGTAA |
| | | TATGGGGTAGTGAAACCACA |
| | | TACTATAATTCAGCTCTCAA |
| | | ATCCAGACTGACCATCATCA |
| | | AGGACAACTCCAAGAGCCAA |
| | | GTTTTCTTAAAAATGAACAG |
| | | TCTGCAAACTGATGACACAG |
| | | CCATTTACTACTGTGCCAAA |
| | | CATTATTACTACGGTGGTAG |
| | | CTATGCTATGGACTACTGGG |
| | | GCCAAGGAACCTCAGTCACC |
| | | GTCTCCTCAACCACGACGCC |
| | | AGCGCCGCGACCACCAACAC |
| | | CGGCGCCCACCATCGCGTCG |
| | | CAGCCCCTGTCCCTGCGCCC |
| | | AGAGGCGTGCCGGCCAGCGG |
| | | CGGGGGGCGCAGTGCACACG |
| | | AGGGGGCTGGACTTCGCCTG |
| | | TGATATCTACATCTGGGCGC |
| | | CCTTGGCCGGGACTTGTGGG |
| | | GTCCTTCTCCTGTCACTGGT |
| | | TATCACCCTTTACTGCAAAC |
| | | GGGGCAGAAAGAAACTCCTG |
| | | TATATATTCAAACAACCATT |
| | | TATGAGACCAGTACAAACTA |
| | | CTCAAGAGGAAGATGGCTGT |
| | | AGCTGCCGATTTCCAGAAGA |
| | | AGAAGAAGGAGGATGTGAAC |
| | | TGAGAGTGAAGTTCAGCAGG |
| | | AGCGCAGACGCCCCCGCGTA |
| | | CAAGCAGGGCCAGAACCAGC |
| | | TCTATAACGAGCTCAATCTA |
| | | GGACGAAGAGAGGAGTACGA |
| | | TGTTTTGGACAAGAGACGTG |
| | | GCCGGGACCCTGAGATGGGG |
| | | GGAAAGCCGAGAAGGAAGAA |
| | | CCCTCAGGAAGGCCTGTACA |
| | | ATGAACTGCAGAAAGATAAG |
| | | ATGGCGGAGGCCTACAGTGA |
| | | GATTGGGATGAAAGGCGAGC |
| | | GCCGGAGGGGCAAGGGGCAC |
| | | GATGGCCTTTACCAGGGTCT |
| | | CAGTACAGCCACCAAGGACA |
| | | CCTACGACGCCCTTCACATG |
| | | CAGGCCCTGCCCCCTCGC |
| 303 | CTL019 | DIQMTQTTSSLSASLGDRVT |
| | scFv | ISCRASQDISKYLNWYQQKP |
| | domain | DGTVKLLIYHTSRLHSGVPS |
| | | RFSGSGSGTDYSLTISNLEQ |
| | | EDIATYFCQQGNTLPYTFGG |
| | | GTKLEITGGGGSGGGGSGGG |
| | | GSEVKLQESGPGLVAPSQSL |
| | | SVTCTVSGVSLPDYGVSWIR |
| | | QPPRKGLEWLGVIWGSETTY |
| | | YNSALKSRLTIIKDNSKSQV |
| | | FLKMNSLQTDDTAIYYCAKH |
| | | YYYGGSYAMDYWGQGTSVTV |
| | | SS |
| Humanized | | |
| CAR2 | | |
| 295 | HCDR1 | DYGVS |
| | (Kabat) | |
| 304 | HCDR2 | VIWGSETTYYQSSLKS |
| | (Kabat) | |
| 297 | HCDR3 | HYYYGGSYAMDY |
| | (Kabat) | |
| 298 | LCDR1 | RASQDISKYLN |
| | (Kabat) | |
| 299 | LCDR2 | HTSRLHS |
| | (Kabat) | |
| 300 | LCDR3 | QQGNTLPYT |
| | (Kabat) | |
| 293 | CAR2 scFv | EIVMTQSPATLSLSPGERAT |
| | domain-aa | LSCRASQDISKYLNWYQQKP |
| | (Linker is | GQAPRLLIYHTSRLHSGIPA |
| | underlined) | RFSGSGSGTDYTLTISSLQP |
| | | EDFAVYFCQQGNTLPYTFGQ |
| | | GTKLEIKGGGGSGGGGSGGG |
| | | GSQVQLQESGPGLVKPSETL |
| | | SLTCTVSGVSLPDYGVSWIR |
| | | QPPGKGLEWIGVIWGSETTY |
| | | YQSSLKSRVTISKDNSKNQV |
| | | SLKLSSVTAADTAVYYCAKH |
| | | YYYGGSYAMDYWGQGTLVTV |
| | | SS |
| 305 | CAR2 scFv | atggccctccctgtcaccgc |
| | domain-nt | cctgctgcttccgctggctc |
| | | ttctgctccacgccgctegg |
| | | cccgaaattgtgatgaccca |
| | | gtcacccgccactcttagcc |
| | | tttcacccggtgagcgcgca |
| | | accctgtcttgcagagcctc |
| | | ccaagacatctcaaaatacc |
| | | ttaattggtatcaacagaag |
| | | cccggacaggctcctcgcct |
| | | tctgatctaccacaccagcc |
| | | ggctccattctggaatccct |
| | | gccaggttcagcggtagcgg |
| | | atctgggaccgactacaccc |
| | | tcactatcagctcactgcag |
| | | ccagaggacttcgctgtcta |
| | | tttctgtcagcaagggaaca |
| | | ccctgccctacacctttgga |
| | | cagggcaccaagctcgagat |
| | | taaaggtggaggtggcagcg |
| | | gaggaggtgggtccggcggt |
| | | ggaggaagccaggtccaact |
| | | ccaagaaagcggaccgggtc |
| | | ttgtgaagccatcagaaact |
| | | ctttcactgacttgtactgt |
| | | gagcggagtgtctctccccg |
| | | attacggggtgtcttggatc |
| | | agacagccaccggggaaggg |
| | | tctggaatggattggagtga |
| | | tttggggctctgagactact |
| | | tactaccaatcatccctcaa |
| | | gtcacgcgtcaccatctcaa |
| | | aggacaactctaagaatcag |
| | | gtgtcactgaaactgtcatc |
| | | tgtgaccgcagccgacaccg |
| | | ccgtgtactattgcgctaag |
| | | cattactattatggcgggag |
| | | ctacgcaatggattactggg |
| | | gacagggtactctggtcacc |
| | | gtgtccagccaccaccatca |
| | | tcaccatcaccat |
| 306 | CAR 2- | MALPVTALLLPLALLLHAAR |
| | Full-aa | PEIVMTQSPATLSLSPGERA |
| | | TLSCRASQDISKYLNWYQQK |
| | | PGQAPRLLIYHTSRLHSGIP |
| | | ARFSGSGSGTDYTLTISSLQ |
| | | PEDFAVYFCQQGNTLPYTFG |
| | | QGTKLEIKGGGGSGGGGSGG |
| | | GGSQVQLQESGPGLVKPSET |
| | | LSLTCTVSGVSLPDYGVSWI |
| | | RQPPGKGLEWIGVIWGSETT |
| | | YYQSSLKSRVTISKDNSKNQ |
| | | VSLKLSSVTAADTAVYYCAK |
| | | HYYYGGSYAMDYWGQGTLVT |
| | | VSSTTTPAPRPPTPAPTIAS |
| | | QPLSLRPEACRPAAGGAVHT |
| | | RGLDFACDIYIWAPLAGTCG |
| | | VLLLSLVITLYCKRGRKKLL |
| | | YIFKQPFMRPVQTTQEEDGC |
| | | SCRFPEEEEGGCELRVKFSR |
| | | SADAPAYKQGQNQLYNELNL |
| | | GRREEYDVLDKRRGRDPEMG |
| | | GKPRRKNPQEGLYNELQKDK |
| | | MAEAYSEIGMKGERRRGKGH |
| | | DGLYQGLSTATKDTYDALHM |
| | | QALPPR |
| 307 | CAR 2- | atggccctccctgtcaccgc |
| | Full-nt | cctgctgcttccgctggctc |
| | | ttctgctccacgccgctcgg |
| | | cccgaaattgtgatgaccca |
| | | gtcacccgccactcttagcc |
| | | tttcacccggtgagcgcgca |
| | | accctgtcttgcagagcctc |
| | | ccaagacatctcaaaatacc |
| | | ttaattggtatcaacagaag |
| | | cccggacaggctcctcgcct |
| | | tctgatctaccacaccagcc |
| | | ggctccattctggaatccct |
| | | gccaggttcagcggtagcgg |
| | | atctgggaccgactacaccc |
| | | tcactatcagctcactgcag |
| | | ccagaggacttcgctgtcta |
| | | tttctgtcagcaagggaaca |
| | | ccctgccctacacctttgga |
| | | cagggcaccaagctcgagat |
| | | taaaggtggaggtggcagcg |
| | | gaggaggtgggtccggcggt |
| | | ggaggaagccaggtccaact |
| | | ccaagaaagcggaccgggtc |
| | | ttgtgaagccatcagaaact |
| | | ctttcactgacttgtactgt |
| | | gagcggagtgtctctccccg |
| | | attacggggtgtcttggatc |
| | | agacagccaccggggaaggg |
| | | tctggaatggattggagtga |
| | | tttggggctctgagactact |
| | | tactaccaatcatccctcaa |
| | | gtcacgcgtcaccatctcaa |
| | | aggacaactctaagaatcag |
| | | gtgtcactgaaactgtcatc |
| | | tgtgaccgcagccgacaccg |
| | | ccgtgtactattgcgctaag |
| | | cattactattatggcgggag |
| | | ctacgcaatggattactggg |
| | | gacagggtactctggtcacc |
| | | gtgtccagcaccactacccc |
| | | agcaccgaggccacccaccc |
| | | cggctcctaccatcgcctcc |
| | | cagcctctgtccctgcgtcc |
| | | ggaggcatgtagacccgcag |
| | | ctggtggggccgtgcatacc |
| | | cggggtcttgacttcgcctg |
| | | cgatatctacatttgggccc |
| | | ctctggctggtacttgcggg |
| | | gtcctgctgctttcactcgt |
| | | gatcactctttactgtaagc |
| | | gcggtcggaagaagctgctg |
| | | tacatctttaagcaaccctt |
| | | catgaggcctgtgcagacta |
| | | ctcaagaggaggacggctgt |
| | | tcatgccggttcccagagga |
| | | ggaggaaggcggctgcgaac |
| | | tgcgcgtgaaattcagccgc |
| | | agcgcagatgctccagccta |
| | | caagcaggggcagaaccagc |
| | | tctacaacgaactcaatctt |
| | | ggtcggagagaggagtacga |
| | | cgtgctggacaagcggagag |
| | | gacgggacccagaaatgggc |
| | | gggaagccgcgcagaaagaa |
| | | tccccaagagggcctgtaca |
| | | acgagctccaaaaggataag |
| | | atggcagaagcctatagcga |
| | | gattggtatgaaaggggaac |
| | | gcagaagaggcaaaggccac |
| | | gacggactgtaccagggact |
| | | cagcaccgccaccaaggaca |
| | | cctatgacgctcttcacatg |
| | | caggccctgccgcctcgg |
| 349 | CAR 2A- | MALPVTALLLPLALLLHAAR |
| | Full amino | PEIVMTQSPATLSLSPGERA |
| | acid | TLSCRASQDISKYLNWYQQK |
| | sequence; | PGQAPRLLIYHTSRLHSGIP |
| | signal | ARFSGSGSGTDYTLTISSLQ |
| | peptide | PEDFAVYFCQQGNTLPYTFG |
| | underlined | QGTKLEIKGGGGSGGGGSGG |
| | | GGSQVQLQESGPGLVKPSET |
| | | LSLTCTVSGVSLPDYGVSWI |
| | | RQPPGKGLEWIGVIWGSETT |
| | | YYQSSLKSRVTISKDNSKNQ |
| | | VSLKLSSVTAADTAVYYCAK |
| | | HYYYGGSYAMDYWGQGTLVT |
| | | VSSTTTPAPRPPTPAPTIAS |
| | | QPLSLRPEACRPAAGGAVHT |
| | | RGLDFACDIYIWAPLAGTCG |
| | | VLLLSLVITLYCKRGRKKLL |
| | | YIFKQPFMRPVQTTQEEDGC |
| | | CRFPEEEEGGCELRVKFSRS |
| | | ADAPAYQQGQNQLYNELNLG |
| | | RREEYDVLDKRRGRDPEMGG |
| | | KPRRKNPQEGLYNELQKDKM |
| | | AEAYSEIGMKGERRRGKGHD |
| | | GLYQGLSTATKDTYDALHMQ |
| | | ALPPR |
| 225 | CAR 2A- | EIVMTQSPATLSLSPGERAT |
| | amino | LSCRASQDISKYLNWYQQKP |
| | acid | GQAPRLLIYHTSRLHSGIPA |
| | sequence; | RFSGSGSGTDYTLTISSLQP |
| | no signal | EDFAVYFCQQGNTLPYTFGQ |
| | peptide | GTKLEIKGGGGSGGGGSGGG |
| | | GSQVQLQESGPGLVKPSETL |
| | | SLTCTVSGVSLPDYGVSWIR |
| | | QPPGKGLEWIGVIWGSETTY |
| | | YQSSLKSRVTISKDNSKNQV |
| | | SLKLSSVTAADTAVYYCAKH |
| | | YYYGGSYAMDYWGQGTLVTV |
| | | SSTTTPAPRPPTPAPTIASQ |
| | | PLSLRPEACRPAAGGAVHTR |
| | | GLDFACDIYIWAPLAGTCGV |
| | | LLLSLVITLYCKRGRKKLLY |
| | | IFKQPFMRPVQTTQEEDGCS |
| | | CRFPEEEEGGCELRVKFSRS |
| | | ADAPAYQQGQNQLYNELNLG |
| | | RREEYDVLDKRRGRDPEMGG |
| | | KPRRKNPQEGLYNELQKDKM |
| | | AEAYSEIGMKGERRRGKGHD |
| | | GLYQGLSTATKDTYDALHMQ |
| | | ALPPR |
| 354 | CAR 2A | atggccctccctgtcaccgc |
| | full | cctgctgcttccgctggctc |
| | nucleic | ttctgctccacgccgctcgg |
| | acid | cccgaaattgtgatgaccca |
| | sequence; | gtcacccgccactcttagcc |
| | signal | tttcacccggtgagcgcgca |
| | peptid | accctgtcttgcagagcctc |
| | e and | ccaagacatctcaaaatacc |
| | stop codon | ttaattggtatcaacagaag |
| | underlined | cccggacaggctcctcgcct |
| | | tctgatctaccacaccagcc |
| | | ggctccattctggaatccct |
| | | gccaggttcagcggtagcgg |
| | | atctgggaccgactacaccc |
| | | tcactatcagctcactgcag |
| | | ccagaggacttcgctgtcta |
| | | tttctgtcagcaagggaaca |
| | | ccctgccctacacctttgga |
| | | cagggcaccaagctcgagat |
| | | taaaggtggaggtggcagcg |
| | | gaggaggtgggtccggcggt |
| | | ggaggaagccaggtccaact |
| | | ccaagaaagcggaccgggtc |
| | | ttgtgaagccatcagaaact |
| | | ctttcactgacttgtactgt |
| | | gagcggagtgtctctccccg |
| | | attacggggtgtcttggatc |
| | | agacagccaccggggaaggg |
| | | tctggaatggattggagtga |
| | | tttggggctctgagactact |
| | | tactaccaatcatccctcaa |
| | | gtcacgcgtcaccatctcaa |
| | | aggacaactctaagaatcag |
| | | gtgtcactgaaactgtcatc |
| | | tgtgaccgcagccgacaccg |
| | | ccgtgtactattgcgctaag |
| | | cattactattatggcgggag |
| | | ctacgcaatggattactggg |
| | | gacagggtactctggtcacc |
| | | gtgtccagcaccactacccc |
| | | agcaccgaggccacccaccc |
| | | cggctcctaccatcgcctcc |
| | | cagcctctgtccctgcgtcc |
| | | ggaggcatgtagacccgcag |
| | | ctggtggggccgtgcatacc |
| | | cggggtcttgacttcgcctg |
| | | cgatatctacatttgggccc |
| | | ctctggctggtacttgcggg |
| | | gtcctgctgctttcactcgt |
| | | gatcactctttactgtaagc |
| | | gcggtcggaagaagctgctg |
| | | tacatctttaagcaaccctt |
| | | catgaggcctgtgcagacta |
| | | ctcaagaggaggacggctgt |
| | | tcatgccggttcccagagga |
| | | ggaggaaggcggctgcgaac |
| | | tgcgcgtgaaattcagccgc |
| | | agcgcagatgctccagccta |
| | | ccagcaggggcagaaccagc |
| | | tctacaacgaactcaatctt |
| | | ggtcggagagaggagtacga |
| | | cgtgctggacaagcggagag |
| | | gacgggacccagaaatgggc |
| | | gggaagccgcgcagaaagaa |
| | | tccccaagagggcctgtaca |
| | | acgagctccaaaaggataag |
| | | atggcagaagcctatagcga |
| | | gattggtatgaaaggggaac |
| | | gcagaagaggcaaaggccac |
| | | gacggactgtaccagggact |
| | | cagcaccgccaccaaggaca |
| | | cctatgacgctcttcacatg |
| | | caggccctgccgcctcggta |
| | | a |
| 355 | CAR 2A | atggccctccctgtcaccgc |
| | nucleic | cctgctgcttccgctggctc |
| | acid | ttctgctccacgccgctcgg |
| | sequence; | cccgaaattgtgatgaccca |
| | signal | gtcacccgccactcttagcc |
| | peptide | tttcacccggtgagcgcgca |
| | underlined; | accctgtcttgcagagcctc |
| | no stop | ccaagacatctcaaaatacc |
| | codon | ttaattggtatcaacagaag |
| | | cccggacaggctcctcgcct |
| | | tctgatctaccacaccagcc |
| | | ggctccattctggaatccct |
| | | gccaggttcagcggtagcgg |
| | | atctgggaccgactacaccc |
| | | tcactatcagctcactgcag |
| | | ccagaggacttcgctgtcta |
| | | tttctgtcagcaagggaaca |
| | | ccctgccctacacctttgga |
| | | cagggcaccaagctcgagat |
| | | taaaggtggaggtggcagcg |
| | | gaggaggtgggtccggcggt |
| | | ggaggaagccaggtccaact |
| | | ccaagaaagcggaccgggtc |
| | | ttgtgaagccatcagaaact |
| | | ctttcactgacttgtactgt |
| | | gagcggagtgtctctccccg |
| | | attacggggtgtcttggatc |
| | | agacagccaccggggaaggg |
| | | tctggaatggattggagtga |
| | | tttggggctctgagactact |
| | | tactaccaatcatccctcaa |
| | | gtcacgcgtcaccatctcaa |
| | | aggacaactctaagaatcag |
| | | gtgtcactgaaactgtcatc |
| | | tgtgaccgcagccgacaccg |
| | | ccgtgtactattgcgctaag |
| | | cattactattatggcgggag |
| | | ctacgcaatggattactggg |
| | | gacagggtactctggtcacc |
| | | gtgtccagcaccactacccc |
| | | agcaccgaggccacccaccc |
| | | cggctcctaccatcgcctcc |
| | | cagcctctgtccctgcgtcc |
| | | ggaggcatgtagacccgcag |
| | | ctggtggggccgtgcatacc |
| | | cggggtcttgacttcgcctg |
| | | cgatatctacatttgggccc |
| | | ctctggctggtacttgcggg |
| | | gtcctgctgctttcactcgt |
| | | gatcactctttactgtaagc |
| | | gcggtcggaagaagctgctg |
| | | tacatctttaagcaaccctt |
| | | catgaggcctgtgcagacta |
| | | ctcaagaggaggacggctgt |
| | | tcatgccggttcccagagga |
| | | ggaggaaggcggctgcgaac |
| | | tgcgcgtgaaattcagccgc |
| | | agcgcagatgctccagccta |
| | | ccagcaggggcagaaccagc |
| | | tctacaacgaactcaatctt |
| | | ggtcggagagaggagtacga |
| | | cgtgctggacaagcggagag |
| | | gacgggacccagaaatgggc |
| | | gggaagccgcgcagaaagaa |
| | | tccccaagagggcctgtaca |
| | | acgagctccaaaaggataag |
| | | atggcagaagcctatagcga |
| | | gattggtatgaaaggggaac |
| | | gcagaagaggcaaaggccac |
| | | gacggactgtaccagggact |
| | | cagcaccgccaccaaggaca |
| | | cctatgacgctcttcacatg |
| | | caggccctgccgcctcgg |
| 356 | CAR 2A | gaaattgtgatgacccagtc |
| | nucleic | acccgccactcttagccttt |
| | acid | cacccggtgagcgcgcaacc |
| | sequence; | ctgtcttgcagagcctccca |
| | no signal | agacatctcaaaatacctta |
| | peptide; | attggtatcaacagaagccc |
| | stop codon | ggacaggctcctcgccttct |
| | underlined | gatctaccacaccagccggc |
| | | tccattctggaatccctgcc |
| | | aggttcagcggtagcggatc |
| | | tgggaccgactacaccctca |
| | | ctatcagctcactgcagcca |
| | | gaggacttcgctgtctattt |
| | | ctgtcagcaagggaacaccc |
| | | tgccctacacctttggacag |
| | | ggcaccaagctcgagattaa |
| | | aggtggaggtggcagcggag |
| | | gaggtgggtccggcggtgga |
| | | ggaagccaggtccaactcca |
| | | agaaagcggaccgggtcttg |
| | | tgaagccatcagaaactctt |
| | | tcactgacttgtactgtgag |
| | | cggagtgtctctccccgatt |
| | | acggggtgtcttggatcaga |
| | | cagccaccggggaagggtct |
| | | ggaatggattggagtgattt |
| | | ggggctctgagactacttac |
| | | taccaatcatccctcaagtc |
| | | acgcgtcaccatctcaaagg |
| | | acaactctaagaatcaggtg |
| | | tcactgaaactgtcatctgt |
| | | gaccgcagccgacaccgccg |
| | | tgtactattgcgctaagcat |
| | | tactattatggcgggagcta |
| | | cgcaatggattactggggac |
| | | agggtactctggtcaccgtg |
| | | tccagcaccactaccccagc |
| | | accgaggccacccaccccgg |
| | | ctcctaccatcgcctcccag |
| | | cctctgtccctgcgtccgga |
| | | ggcatgtagacccgcagctg |
| | | gtggggccgtgcatacccgg |
| | | ggtcttgacttcgcctgcga |
| | | tatctacatttgggcccctc |
| | | tggctggtacttgcggggtc |
| | | ctgctgctttcactcgtgat |
| | | cactctttactgtaagcgcg |
| | | gtcggaagaagctgctgtac |
| | | atctttaagcaacccttcat |
| | | gaggcctgtgcagactactc |
| | | aagaggaggacggctgttca |
| | | tgccggttcccagaggagga |
| | | ggaaggcggctgcgaactgc |
| | | gcgtgaaattcagccgcagc |
| | | gcagatgctccagcctacca |
| | | gcaggggcagaaccagctct |
| | | acaacgaactcaatcttggt |
| | | cggagagaggagtacgacgt |
| | | gctggacaagcggagaggac |
| | | gggacccagaaatgggcggg |
| | | aagccgcgcagaaagaatcc |
| | | ccaagagggcctgtacaacg |
| | | agctccaaaaggataagatg |
| | | gcagaagcctatagcgagat |
| | | tggtatgaaaggggaacgca |
| | | gaagaggcaaaggccacgac |
| | | ggactgtaccagggactcag |
| | | caccgccaccaaggacacct |
| | | atgacgctcttcacatgcag |
| | | gccctgccgcctcggtaa |
| SEQ ID | CAR 2A | gaaattgtgatgacccagtc |
| NO: 417 | nucleic | acccgccactcttagccttt |
| | acid | cacccggtgagcgcgcaacc |
| | sequence; | ctgtcttgcagagcctccca |
| | no signal | agacatctcaaaatacctta |
| | peptide; no | attggtatcaacagaagccc |
| | stop codon | ggacaggctcctcgccttct |
| | | gatctaccacaccagccggc |
| | | tccattctggaatccctgcc |
| | | aggttcagcggtagcggatc |
| | | tgggaccgactacaccctca |
| | | ctatcagctcactgcagcca |
| | | gaggacttcgctgtctattt |
| | | ctgtcagcaagggaacaccc |
| | | tgccctacacctttggacag |
| | | ggcaccaagctcgagattaa |
| | | aggtggaggtggcagcggag |
| | | gaggtgggtccggcggtgga |
| | | ggaagccaggtccaactcca |
| | | agaaagcggaccgggtcttg |
| | | tgaagccatcagaaactctt |
| | | tcactgacttgtactgtgag |
| | | cggagtgtctctccccgatt |
| | | acggggtgtcttggatcaga |
| | | cagccaccggggaagggtct |
| | | ggaatggattggagtgattt |
| | | ggggctctgagactacttac |
| | | taccaatcatccctcaagtc |
| | | acgcgtcaccatctcaaagg |
| | | acaactctaagaatcaggtg |
| | | tcactgaaactgtcatctgt |
| | | gaccgcagccgacaccgccg |
| | | tgtactattgcgctaagcat |
| | | tactattatggcgggagcta |
| | | cgcaatggattactggggac |
| | | agggtactctggtcaccgtg |
| | | tccagcaccactaccccagc |
| | | accgaggccacccaccccgg |
| | | ctcctaccatcgcctcccag |
| | | cctctgtccctgcgtccgga |
| | | ggcatgtagacccgcagctg |
| | | gtggggccgtgcatacccgg |
| | | ggtcttgacttcgcctgcga |
| | | tatctacatttgggcccctc |
| | | tggctggtacttgcggggtc |
| | | ctgctgctttcactcgtgat |
| | | cactctttactgtaagcgcg |
| | | gtcggaagaagctgctgtac |
| | | atctttaagcaacccttcat |
| | | gaggcctgtgcagactactc |
| | | aagaggaggacggctgttca |
| | | tgccggttcccagaggagga |
| | | ggaaggcggctgcgaactgc |
| | | gcgtgaaattcagccgcagc |
| | | gcagatgctccagcctacca |
| | | gcaggggcagaaccagctct |
| | | acaacgaactcaatcttggt |
| | | cggagagaggagtacgacgt |
| | | gctggacaagcggagaggac |
| | | gggacccagaaatgggcggg |
| | | aagccgcgcagaaagaatcc |
| | | ccaagagggcctgtacaacg |
| | | agctccaaaaggataagatg |
| | | gcagaagcctatagcgagat |
| | | tggtatgaaaggggaacgca |
| | | gaagaggcaaaggccacgac |
| | | ggactgtaccagggactcag |
| | | caccgccaccaaggacacct |
| | | atgacgctcttcacatgcag |
| | | gccctgccgcctcgg |
| SEQ ID | Anti-CD19 | QVQLQESGPGLVKPSETLSL |
| NO: 250 | VH | TCTVSGVSLPDYGVSWIRQP |
| | | PGKGLEWIGVIWGSETTYYQ |
| | | SSLKSRVTISKDNSKNQVSL |
| | | KLSSVTAADTAVYYCAKHYY |
| | | YGGSYAMDYWGQGTLVTVSS |
| SEQ ID | Anti-CD19 | EIVMTQSPATLSLSPGERAT |
| NO: 251 | VL | LSCRASQDISKYLNWYQQKP |
| | | GQAPRLLIYHTSRLHSGIPA |
| | | RFSGSGSGTDYTLTISSLQP |
| | | EDFAVYFCQQGNTLPYTFGQ |
| | | GTKLEIK |
| SEQ ID | VH | QVQLQESGPGLVKPSETLSL |
| NO: 331 | | TCTVSGVSLPDYGVSWIRQP |
| | | PGKCLEWIGVIWGSETTYYQ |
| | | SSLKSRVTISKDNSKNQVSL |
| | | KLSSVTAADTAVYYCAKHYY |
| | | YGGSYAMDYWGQGTLVTVSS |
| SEQ ID | VL | EIVMTQSPATLSLSPGERAT |
| NO: 332 | | LSCRASQDISKYLNWYQQKP |
| | | GQAPRLLIYHTSRLHSGIPA |
| | | RFSGSGSGTDYTLTISSLQP |
| | | EDFAVYFCQQGNTLPYTFGC |
| | | GTKLEIK |

### BCMA CAR

In some embodiments, the CAR-expressing cell described herein is a BCMA CAR-expressing cell (for example, a cell expressing a CAR that binds to human BCMA). Exemplary BCMA CARs can include sequences disclosed in Table 1 or 16 of WO2016/014565, incorporated herein by reference. The BCMA CAR construct can include an optional leader sequence; an optional hinge domain, for example, a CD8 hinge domain; a transmembrane domain, for example, a CD8 transmembrane domain; an intracellular domain, for example, a 4-1BB intracellular domain; and a functional signaling domain, for example, a CD3 zeta domain. In certain embodiments, the domains are contiguous and in the same reading frame to form a single fusion protein. In other embodiments, the domains are in separate polypeptides, for example, as in an RCAR molecule as described herein.

In some embodiments, the BCMA CAR molecule includes one or more CDRs, VH, VL, scFv, or full-length sequences of BCMA-1, BCMA-2, BCMA-3, BCMA-4, BCMA-5, BCMA-6, BCMA-7, BCMA-8, BCMA-9, BCMA-10, BCMA-11, BCMA-12, BCMA-13, BCMA-14, BCMA-15, 149362, 149363, 149364, 149365, 149366, 149367, 149368, 149369, BCMA_EBB-C1978-A4, BCMA_EBB-C1978-G1, BCMA_EBB-C1979-C1, BCMA_EBB-C1978-C7, BCMA_EBB-C1978-D10, BCMA_EBB-C1979-C12, BCMA_EBB-C1980-G4, BCMA_EBB-C1980-D2, BCMA_EBB-C1978-A10, BCMA_EBB-C1978-D4, BCMA_EBB-C1980-A2, BCMA_EBB-C1981-C3, BCMA_EBB-C1978-G4, A7D12.2, C11D5.3, C12A3.2, or C13F12.1 disclosed in WO2016/014565, or a sequence substantially (for example, 95-99%) identical thereto.

Additional exemplary BCMA-targeting sequences that can be used in the anti-BCMA CAR constructs are disclosed in WO 2017/021450, WO 2017/011804, WO 2017/025038, WO 2016/090327, WO 2016/130598, WO 2016/210293, WO 2016/090320, WO 2016/014789, WO 2016/094304, WO 2016/154055, WO 2015/166073, WO 2015/188119, WO 2015/158671, U.S. Pat. Nos. 9,243,058, 8,920,776, 9,273,141, 7,083,785, 9,034,324, US 2007/0049735, US 2015/0284467, US 2015/0051266, US 2015/0344844, US 2016/0131655, US 2016/0297884, US 2016/0297885, US 2017/0051308, US 2017/0051252, US 2017/0051252, WO 2016/020332, WO 2016/087531, WO 2016/079177, WO 2015/172800, WO 2017/008169, U.S. Pat. No. 9,340,621, US 2013/0273055, US 2016/0176973, US 2015/0368351, US 2017/0051068, US 2016/0368988, and US 2015/0232557, herein incorporated by reference in their entirety. In some embodiments, additional exemplary BCMA CAR constructs are generated using the VH and VL sequences from PCT Publication WO2012/0163805 (the contents of which are hereby incorporated by reference in its entirety).

In some embodiments, BCMA CARs comprise a sequence, for example, a CDR, VH, VL, scFv, or full-CAR sequence, disclosed in Tables 3-15, or a sequence having at least 80%, 85%, 90%, 95%, or 99% identity thereto. In some embodiments, the antigen binding domain comprises a human antibody or a human antibody fragment. In some embodiments, the human anti-BCMA binding domain comprises one or more (for example, all three) LC CDR1, LC CDR2, and LC CDR3 of a human anti-BCMA binding domain described herein (for example, in Tables 3-10 and 12-15), and/or one or more (for example, all three) HC CDR1, HC CDR2, and HC CDR3 of a human anti-BCMA binding domain described herein (for example, in Tables 3-10 and 12-15). In some embodiments, the human anti-BCMA binding domain comprises a human VL described herein (for example, in Tables 3, 7, and 12) and/or a human VH described herein (for example, in Tables 3, 7, and 12). In some embodiments, the anti-BCMA binding domain is a scFv comprising a VL and a VH of an amino acid sequence of Tables 3, 7, and 12. In some embodiments, the anti-BCMA binding domain (for example, an scFv) comprises: a VL comprising an amino acid sequence having at least one, two or three modifications (for example, substitutions, for example, conservative substitutions) but not more than 30, 20 or 10 modifications (for example, substitutions, for example, conservative substitutions) of an amino acid sequence provided in Tables 3, 7, and 12, or a sequence with 95-99% identity with an amino acid sequence of Tables 3, 7, and 12, and/or a VH comprising an amino acid sequence having at least one, two or three modifications (for example, substitutions, for example, conservative substitutions) but not more than 30, 20 or 10 modifications (for example, substitutions, for example, conservative substitutions) of an amino acid sequence provided in Tables 3, 7, and 12, or a sequence with 95-99% identity to an amino acid sequence of Tables 3, 7, and 12.

TABLE 3
Amino acid and nucleic acid sequences
of exemplary PALLAS-derived anti-
BCMA mlecules

| SEQ ID | Name/ | |
|---|---|---|
| NO | Description | Sequence |
| R1B6 | | |
| SEQ ID | HCDR1 | SYAMS |
| NO: 44 | (Kabat) | |
| SEQ ID | HCDR2 | AISGSGGSTYYADSVKG |
| NO: 45 | (Kabat) | |
| SEQ ID | HCDR3 | REWVPYDVSWYFDY |
| NO: 46 | (Kabat) | |
| SEQ ID | HCDR1 | GFTFSSY |
| NO: 47 | (Chothia) | |
| SEQ ID | HCDR2 | SGSGGS |
| NO: 48 | (Chothia) | |
| SEQ ID | HCDR3 | REWVPYDVSWYFDY |
| NO: 46 | (Chothia) | |
| SEQ ID | HCDR1 | GFTFSSYA |
| NO: 49 | (IMGT) | |
| SEQ ID | HCDR2 | ISGSGGST |
| NO: 50 | (IMGT) | |
| SEQ ID | HCDR3 | ARREWVPYDVSWYFDY |
| NO: 51 | (IMGT) | |
| SEQ ID | VH | EVQLLESGGGLVQPGGSLRLSCAAS |
| NO: 52 | | GFTFSSYAMSWVRQAPGKGLEWVSA |
| | | ISGSGGSTYYADSVKGRFTISRDNS |
| | | KNTLYLQMNSLRAEDTAVYYCARRE |
| | | WVPYDVSWYFDYWGQGTLVTVSS |
| SEQ ID | DNA VH | GAAGTGCAGTTGCTGGAGTCAGGCG |
| NO: 53 | | GAGGACTGGTGCAGCCCGGAGGATC |
| | | GCTTCGCTTGAGCTGCGCAGCCTCA |
| | | GGCTTTACCTTCTCCTCCTACGCCA |
| | | TGTCCTGGGTCAGACAGGCTCCCGG |
| | | GAAGGGACTGGAATGGGTGTCCGCC |
| | | ATTAGCGGTTCCGGCGGAAGCACTT |
| | | ACTATGCCGACTCTGTGAAGGGCCG |
| | | CTTCACTATCTCCCGGGACAACTCC |
| | | AAGAACACCCTGTATCTCCAAATGA |
| | | ATTCCCTGAGGGCCGAAGATACCGC |
| | | GGTGTACTACTGCGCTAGACGGGAG |
| | | TGGGTGCCCTACGATGTCAGCTGGT |
| | | ACTTCGACTACTGGGGACAGGGCAC |
| | | TCTCGTGACTGTGTCCTCC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPLT |
| NO: 56 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPL |
| NO: 59 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPLT |
| NO: 56 | (IMGT) | |
| SEQ ID | VL | DIQMTQSPSSLSASVGDRVTITCRA |
| NO: 61 | | SQSISSYLNWYQQKPGKAPKLLIYA |
| | | ASSLQSGVPSRFSGSGSGTDFTLTI |
| | | SSLQPEDFATYYCQQSYSTPLTFGQ |
| | | GTKVEIK |
| SEQ ID | DNA VL | GACATTCAAATGACTCAGTCCCCGT |
| NO: 62 | | CCTCCCTCTCCGCCTCCGTGGGAGA |
| | | TCGCGTCACGATCACGTGCAGGGCC |
| | | AGCCAGAGCATCTCCAGCTACCTGA |
| | | ACTGGTACCAGCAGAAGCCAGGGAA |
| | | GGCACCGAAGCTCCTGATCTACGCC |
| | | GCTAGCTCGCTGCAGTCCGGCGTCC |
| | | CTTCACGGTTCTCGGGATCGGGCTC |
| | | AGGCACCGACTTCACCCTGACCATT |
| | | AGCAGCCTGCAGCCGGAGGACTTCG |
| | | CGACATACTACTGTCAGCAGTCATA |
| | | CTCCACCCCTCTGACCTTCGGCCAA |
| | | GGGACCAAAGTGGAGATCAAG |
| SEQ ID | Linker | GGGGSGGGGSGGGGSGGGGS |
| NO: 63 | | |
| SEQ ID | scFv | EVQLLESGGGLVQPGGSLRLSCAAS |
| NO: 64 | (VH- | GFTFSSYAMSWVRQAPGKGLEWVSA |
| | linker- | ISGSGGSTYYADSVKGRFTISRDNS |
| | VL) | KNTLYLQMNSLRAEDTAVYYCARRE |
| | | WVPYDVSWYFDYWGQGTLVTVSSGG |
| | | GGSGGGGSGGGGSGGGGSDIQMTQS |
| | | PSSLSASVGDRVTITCRASQSISSY |
| | | LNWYQQKPGKAPKLLIYAASSLQSG |
| | | VPSRFSGSGSGTDFTLTISSLQPED |
| | | FATYYCQQSYSTPLTFGQGTKVEIK |
| SEQ ID | DNA | GAAGTGCAGTTGCTGGAGTCAGGCG |
| NO: 65 | scFv | GAGGACTGGTGCAGCCCGGAGGATC |
| | | GCTTCGCTTGAGCTGCGCAGCCTCA |
| | | GGCTTTACCTTCTCCTCCTACGCCA |
| | | TGTCCTGGGTCAGACAGGCTCCCGG |
| | | GAAGGGACTGGAATGGGTGTCCGCC |
| | | ATTAGCGGTTCCGGCGGAAGCACTT |
| | | ACTATGCCGACTCTGTGAAGGGCCG |
| | | CTTCACTATCTCCCGGGACAACTCC |
| | | AAGAACACCCTGTATCTCCAAATGA |
| | | ATTCCCTGAGGGCCGAAGATACCGC |
| | | GGTGTACTACTGCGCTAGACGGGAG |
| | | TGGGTGCCCTACGATGTCAGCTGGT |
| | | ACTTCGACTACTGGGGACAGGGCAC |
| | | TCTCGTGACTGTGTCCTCCGGTGGT |
| | | GGTGGATCGGGGGGTGGTGGTTCGG |
| | | GCGGAGGAGGATCTGGAGGAGGAGG |
| | | GTCGGACATTCAAATGACTCAGTCC |
| | | CCGTCCTCCCTCTCCGCCTCCGTGG |
| | | GAGATCGCGTCACGATCACGTGCAG |
| | | GGCCAGCCAGAGCATCTCCAGCTAC |
| | | CTGAACTGGTACCAGCAGAAGCCAG |
| | | GGAAGGCACCGAAGCTCCTGATCTA |
| | | CGCCGCTAGCTCGCTGCAGTCCGGC |
| | | GTCCCTTCACGGTTCTCGGGATCGG |
| | | GCTCAGGCACCGACTTCACCCTGAC |
| | | CATTAGCAGCCTGCAGCCGGAGGAC |
| | | TTCGCGACATACTACTGTCAGCAGT |
| | | CATACTCCACCCCTCTGACCTTCGG |
| | | CCAAGGGACCAAAGTGGAGATCAAG |
| SEQ ID | Full | EVQLLESGGGLVQPGGSLRLSCAAS |
| NO: 66 | CAR | GFTFSSYAMSWVRQAPGKGLEWVSA |
| | amino | ISGSGGSTYYADSVKGRFTISRDNS |
| | acid | KNTLYLQMNSLRAEDTAVYYCARRE |
| | sequence | WVPYDVSWYFDYWGQGTLVTVSSGG |
| | | GGSGGGGSGGGGSGGGGSDIQMTQS |
| | | PSSLSASVGDRVTITCRASQSISSY |
| | | LNWYQQKPGKAPKLLIYAASSLQSG |
| | | VPSRFSGSGSGTDFTLTISSLQPED |
| | | FATYYCQQSYSTPLTFGQGTKVEIK |
| | | TTTPAPRPPTPAPTIASQPLSLRPE |
| | | ACRPAAGGAVHTRGLDFACDIYIWA |
| | | PLAGTCGVLLLSLVITLYCKRGRKK |
| | | LLYIFKQPFMRPVQTTQEEDGCSCR |
| | | FPEEEEGGCELRVKFSRSADAPAYQ |
| | | QGQNQLYNELNLGRREEYDVLDKRR |
| | | GRDPEMGGKPRRKNPQEGLYNELQK |
| | | DKMAEAYSEIGMKGERRRGKGHDGL |
| | | YQGLSTATKDTYDALHMQALPPR |
| SEQ ID | Full | GAAGTGCAGTTGCTGGAGTCAGGCG |
| NO: 67 | CAR | GAGGACTGGTGCAGCCCGGAGGATC |
| | DNA | GCTTCGCTTGAGCTGCGCAGCCTCA |
| | sequence | GGCTTTACCTTCTCCTCCTACGCCA |
| | | TGTCCTGGGTCAGACAGGCTCCCGG |
| | | GAAGGGACTGGAATGGGTGTCCGCC |
| | | ATTAGCGGTTCCGGCGGAAGCACTT |
| | | ACTATGCCGACTCTGTGAAGGGCCG |
| | | CTTCACTATCTCCCGGGACAACTCC |
| | | AAGAACACCCTGTATCTCCAAATGA |
| | | ATTCCCTGAGGGCCGAAGATACCGC |
| | | GGTGTACTACTGCGCTAGACGGGAG |
| | | TGGGTGCCCTACGATGTCAGCTGGT |
| | | ACTTCGACTACTGGGGACAGGGCAC |
| | | TCTCGTGACTGTGTCCTCCGGTGGT |
| | | GGTGGATCGGGGGGTGGTGGTTCGG |
| | | GCGGAGGAGGATCTGGAGGAGGAGG |
| | | GTCGGACATTCAAATGACTCAGTCC |
| | | CCGTCCTCCCTCTCCGCCTCCGTGG |
| | | GAGATCGCGTCACGATCACGTGCAG |
| | | GGCCAGCCAGAGCATCTCCAGCTAC |
| | | CTGAACTGGTACCAGCAGAAGCCAG |
| | | GGAAGGCACCGAAGCTCCTGATCTA |
| | | CGCCGCTAGCTCGCTGCAGTCCGGC |
| | | GTCCCTTCACGGTTCTCGGGATCGG |
| | | GCTCAGGCACCGACTTCACCCTGAC |
| | | CATTAGCAGCCTGCAGCCGGAGGAC |
| | | TTCGCGACATACTACTGTCAGCAGT |
| | | CATACTCCACCCCTCTGACCTTCGG |
| | | CCAAGGGACCAAAGTGGAGATCAAG |
| | | ACCACTACCCCAGCACCGAGGCCAC |
| | | CCACCCCGGCTCCTACCATCGCCTC |
| | | CCAGCCTCTGTCCCTGCGTCCGGAG |
| | | GCATGTAGACCCGCAGCTGGTGGGG |
| | | CCGTGCATACCCGGGGTCTTGACTT |
| | | CGCCTGCGATATCTACATTTGGGCC |
| | | CCTCTGGCTGGTACTTGCGGGGTCC |
| | | TGCTGCTTTCACTCGTGATCACTCT |
| | | TTACTGTAAGCGCGGTCGGAAGAAG |
| | | CTGCTGTACATCTTTAAGCAACCCT |
| | | TCATGAGGCCTGTGCAGACTACTCA |
| | | AGAGGAGGACGGCTGTTCATGCCGG |
| | | TTCCCAGAGGAGGAGGAAGGCGGCT |
| | | GCGAACTGCGCGTGAAATTCAGCCG |
| | | CAGCGCAGATGCTCCAGCCTACCAG |
| | | CAGGGGCAGAACCAGCTCTACAACG |
| | | AACTCAATCTTGGTCGGAGAGAGGA |
| | | GTACGACGTGCTGGACAAGCGGAGA |
| | | GGACGGGACCCAGAAATGGGCGGGA |
| | | AGCCGCGCAGAAAGAATCCCCAAGA |
| | | GGGCCTGTACAACGAGCTCCAAAAG |
| | | GATAAGATGGCAGAAGCCTATAGCG |
| | | AGATTGGTATGAAAGGGGAACGCAG |
| | | AAGAGGCAAAGGCCACGACGGACTG |
| | | TACCAGGGACTCAGCACCGCCACCA |
| | | AGGACACCTATGACGCTCTTCACAT |
| | | GCAGGCCCTGCCGCCTCGG |
| R1F2 | | |
| SEQ ID | HCDR1 | SYAMS |
| NO: 44 | (Kabat) | |
| SEQ ID | HCDR2 | AISGSGGSTYYADSVKG |
| NO: 45 | (Kabat) | |
| SEQ ID | HCDR3 | REWWYDDWYLDY |
| NO: 68 | (Kabat) | |
| SEQ ID | HCDR1 | GFTFSSY |
| NO: 47 | (Chothia) | |
| SEQ ID | HCDR2 | SGSGGS |
| NO: 48 | (Chothia) | |
| SEQ ID | HCDR3 | REWWYDDWYLDY |
| NO: 68 | (Chothia) | |
| SEQ ID | HCDR1 | GFTFSSYA |
| NO: 49 | (IMGT) | |
| SEQ ID | HCDR2 | ISGSGGST |
| NO: 50 | (IMGT) | |
| SEQ ID | HCDR3 | ARREWWYDDWYLDY |
| NO: 69 | (IMGT) | |
| SEQ ID | VH | EVQLLESGGGLVQPGGSLRLSCAAS |
| NO: 70 | | GFTFSSYAMSWVRQAPGKGLEWVSA |
| | | ISGSGGSTYYADSVKGRFTISRDNS |
| | | KNTLYLQMNSLRAEDTAVYYCARRE |
| | | WWYDDWYLDYWGQGTLVTVSS |
| SEQ ID | DNA VH | GAAGTGCAGTTGCTGGAGTCAGGCG |
| NO: 71 | | GAGGACTGGTGCAGCCCGGAGGATC |
| | | GCTTCGCTTGAGCTGCGCAGCCTCA |
| | | GGCTTTACCTTCTCCTCCTACGCCA |
| | | TGTCCTGGGTCAGACAGGCTCCCGG |
| | | GAAGGGACTGGAATGGGTGTCCGCC |
| | | ATTAGCGGTTCCGGCGGAAGCACTT |
| | | ACTATGCCGACTCTGTGAAGGGCCG |
| | | CTTCACTATCTCCCGGGACAACTCC |
| | | AAGAACACCCTGTATCTCCAAATGA |
| | | ATTCCCTGAGGGCCGAAGATACCGC |
| | | GGTGTACTACTGCGCTAGACGGGAG |
| | | TGGTGGTACGACGATTGGTACCTGG |
| | | ACTACTGGGGACAGGGCACTCTCGT |
| | | GACTGTGTCCTCC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPLT |
| NO: 56 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPL |
| NO: 59 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPLT |
| NO: 56 | (IMGT) | |
| SEQ ID | VL | DIQMTQSPSSLSASVGDRVTITCRA |
| NO: 61 | | SQSISSYLNWYQQKPGKAPKLLIYA |
| | | ASSLQSGVPSRFSGSGSGTDFTLTI |
| | | SSLQPEDFATYYCQQSYSTPLTFGQ |
| | | GTKVEIK |
| SEQ ID | DNA VL | GACATTCAAATGACTCAGTCCCCGT |
| NO: 62 | | CCTCCCTCTCCGCCTCCGTGGGAGA |
| | | TCGCGTCACGATCACGTGCAGGGCC |
| | | AGCCAGAGCATCTCCAGCTACCTGA |
| | | ACTGGTACCAGCAGAAGCCAGGGAA |
| | | GGCACCGAAGCTCCTGATCTACGCC |
| | | GCTAGCTCGCTGCAGTCCGGCGTCC |
| | | CTTCACGGTTCTCGGGATCGGGCTC |
| | | AGGCACCGACTTCACCCTGACCATT |
| | | AGCAGCCTGCAGCCGGAGGACTTCG |
| | | CGACATACTACTGTCAGCAGTCATA |
| | | CTCCACCCCTCTGACCTTCGGCCAA |
| | | GGGACCAAAGTGGAGATCAAG |
| SEQ ID | Linker | GGGGSGGGGSGGGGSGGGGS |
| NO: 63 | | |
| SEQ ID | scFv | EVQLLESGGGLVQPGGSLRLSCAAS |
| NO: 72 | (VH- | GFTFSSYAMSWVRQAPGKGLEWVSA |
| | linker- | ISGSGGSTYYADSVKGRFTISRDNS |
| | VL) | KNTLYLQMNSLRAEDTAVYYCARRE |
| | | WWYDDWYLDYWGQGTLVTVSSGGGG |
| | | SGGGGSGGGGSGGGGSDIQMTQSPS |
| | | SLSASVGDRVTITCRASQSISSYLN |
| | | WYQQKPGKAPKLLIYAASSLQSGVP |
| | | SRFSGSGSGTDFTLTISSLQPEDFA |
| | | TYYCQQSYSTPLTFGQGTKVEIK |
| SEQ ID | DNA | GAAGTGCAGTTGCTGGAGTCAGGCG |
| NO: 73 | scFv | GAGGACTGGTGCAGCCCGGAGGATC |
| | | GCTTCGCTTGAGCTGCGCAGCCTCA |
| | | GGCTTTACCTTCTCCTCCTACGCCA |
| | | TGTCCTGGGTCAGACAGGCTCCCGG |
| | | GAAGGGACTGGAATGGGTGTCCGCC |
| | | ATTAGCGGTTCCGGCGGAAGCACTT |
| | | ACTATGCCGACTCTGTGAAGGGCCG |
| | | CTTCACTATCTCCCGGGACAACTCC |
| | | AAGAACACCCTGTATCTCCAAATGA |
| | | ATTCCCTGAGGGCCGAAGATACCGC |
| | | GGTGTACTACTGCGCTAGACGGGAG |
| | | TGGTGGTACGACGATTGGTACCTGG |
| | | ACTACTGGGGACAGGGCACTCTCGT |
| | | GACTGTGTCCTCCGGTGGTGGTGGA |
| | | TCGGGGGGTGGTGGTTCGGGCGGAG |
| | | GAGGATCTGGAGGAGGAGGGTCGGA |
| | | CATTCAAATGACTCAGTCCCCGTCC |
| | | TCCCTCTCCGCCTCCGTGGGAGATC |
| | | GCGTCACGATCACGTGCAGGGCCAG |
| | | CCAGAGCATCTCCAGCTACCTGAAC |
| | | TGGTACCAGCAGAAGCCAGGGAAGG |
| | | CACCGAAGCTCCTGATCTACGCCGC |
| | | TAGCTCGCTGCAGTCCGGCGTCCCT |
| | | TCACGGTTCTCGGGATCGGGCTCAG |
| | | GCACCGACTTCACCCTGACCATTAG |
| | | CAGCCTGCAGCCGGAGGACTTCGCG |
| | | ACATACTACTGTCAGCAGTCATACT |
| | | CCACCCCTCTGACCTTCGGCCAAGG |
| | | GACCAAAGTGGAGATCAAG |
| SEQ ID | Full | EVQLLESGGGLVQPGGSLRLSCAAS |
| NO: 74 | CAR | GFTFSSYAMSWVRQAPGKGLEWVSA |
| SEQ ID | amino | ISGSGGSTYYADSVKGRFTISRDNS |
| NO: 75 | acid | KNTLYLQMNSLRAEDTAVYYCARRE |
| | sequence | WWYDDWYLDYWGQGTLVTVSSGGGG |
| | | SGGGGSGGGGSGGGGSDIQMTQSPS |
| | | SLSASVGDRVTITCRASQSISSYLN |
| | | WYQQKPGKAPKLLIYAASSLQSGVP |
| | | SRFSGSGSGTDFTLTISSLQPEDFA |
| | | TYYCQQSYSTPLTFGQGTKVEIKTT |
| | | TPAPRPPTPAPTIASQPLSLRPEAC |
| | | RPAAGGAVHTRGLDFACDIYIWAPL |
| | | AGTCGVLLLSLVITLYCKRGRKKLL |
| | | YIFKQPFMRPVQTTQEEDGCSCRFP |
| | | EEEEGGCELRVKFSRSADAPAYQQG |
| | | QNQLYNELNLGRREEYDVLDKRRGR |
| | | DPEMGGKPRRKNPQEGLYNELQKDK |
| | | MAEAYSEIGMKGERRRGKGHDGLYQ |
| | | GLSTATKDTYDALHMQALPPR |
| | Full | GAAGTGCAGTTGCTGGAGTCAGGCG |
| | CAR | GAGGACTGGTGCAGCCCGGAGGATC |
| | DNA | GCTTCGCTTGAGCTGCGCAGCCTCA |
| | sequence | GGCTTTACCTTCTCCTCCTACGCCA |
| | | TGTCCTGGGTCAGACAGGCTCCCGG |
| | | GAAGGGACTGGAATGGGTGTCCGCC |
| | | ATTAGCGGTTCCGGCGGAAGCACTT |
| | | ACTATGCCGACTCTGTGAAGGGCCG |
| | | CTTCACTATCTCCCGGGACAACTCC |
| | | AAGAACACCCTGTATCTCCAAATGA |
| | | ATTCCCTGAGGGCCGAAGATACCGC |
| | | GGTGTACTACTGCGCTAGACGGGAG |
| | | TGGTGGTACGACGATTGGTACCTGG |
| | | ACTACTGGGGACAGGGCACTCTCGT |
| | | GACTGTGTCCTCCGGTGGTGGTGGA |
| | | TCGGGGGGTGGTGGTTCGGGCGGAG |
| | | GAGGATCTGGAGGAGGAGGGTCGGA |
| | | CATTCAAATGACTCAGTCCCCGTCC |
| | | TCCCTCTCCGCCTCCGTGGGAGATC |
| | | GCGTCACGATCACGTGCAGGGCCAG |
| | | CCAGAGCATCTCCAGCTACCTGAAC |
| | | TGGTACCAGCAGAAGCCAGGGAAGG |
| | | CACCGAAGCTCCTGATCTACGCCGC |
| | | TAGCTCGCTGCAGTCCGGCGTCCCT |
| | | TCACGGTTCTCGGGATCGGGCTCAG |
| | | GCACCGACTTCACCCTGACCATTAG |
| | | CAGCCTGCAGCCGGAGGACTTCGCG |
| | | ACATACTACTGTCAGCAGTCATACT |
| | | CCACCCCTCTGACCTTCGGCCAAGG |
| | | GACCAAAGTGGAGATCAAGACCACT |
| | | ACCCCAGCACCGAGGCCACCCACCC |
| | | CGGCTCCTACCATCGCCTCCCAGCC |
| | | TCTGTCCCTGCGTCCGGAGGCATGT |
| | | AGACCCGCAGCTGGTGGGGCCGTGC |
| | | ATACCCGGGGTCTTGACTTCGCCTG |
| | | CGATATCTACATTTGGGCCCCTCTG |
| | | GCTGGTACTTGCGGGGTCCTGCTGC |
| | | TTTCACTCGTGATCACTCTTTACTG |
| | | TAAGCGCGGTCGGAAGAAGCTGCTG |
| | | TACATCTTTAAGCAACCCTTCATGA |
| | | GGCCTGTGCAGACTACTCAAGAGGA |
| | | GGACGGCTGTTCATGCCGGTTCCCA |
| | | GAGGAGGAGGAAGGCGGCTGCGAAC |
| | | TGCGCGTGAAATTCAGCCGCAGCGC |
| | | AGATGCTCCAGCCTACCAGCAGGGG |
| | | CAGAACCAGCTCTACAACGAACTCA |
| | | ATCTTGGTCGGAGAGAGGAGTACGA |
| | | CGTGCTGGACAAGCGGAGAGGACGG |
| | | GACCCAGAAATGGGCGGGAAGCCGC |
| | | GCAGAAAGAATCCCCAAGAGGGCCT |
| | | GTACAACGAGCTCCAAAAGGATAAG |
| | | ATGGCAGAAGCCTATAGCGAGATTG |
| | | GTATGAAAGGGGAACGCAGAAGAGG |
| | | CAAAGGCCACGACGGACTGTACCAG |
| | | GGACTCAGCACCGCCACCAAGGACA |
| | | CCTATGACGCTCTTCACATGCAGGC |
| | | CCTGCCGCCTCGG |
| R1G5 | | |
| SEQ ID | HCDR1 | SYAMS |
| NO: 44 | (Kabat) | |
| SEQ ID | HCDR2 | AISGSGGSTYYADSVKG |
| NO: 45 | (Kabat) | |
| SEQ ID | HCDR3 | REWWGESWLFDY |
| NO: 76 | (Kabat) | |
| SEQ ID | HCDR1 | GFTFSSY |
| NO: 47 | (Chothia) | |
| SEQ ID | HCDR2 | SGSGGS |
| NO: 48 | (Chothia) | |
| SEQ ID | HCDR3 | REWWGESWLFDY |
| NO: 76 | (Chothia) | |
| SEQ ID | HCDR1 | GFTFSSYA |
| NO: 49 | (IMGT) | |
| SEQ ID | HCDR2 | ISGSGGST |
| NO: 50 | (IMGT) | |
| SEQ ID | HCDR3 | ARREWWGESWLFDY |
| NO: 77 | (IMGT) | |
| SEQ ID | VH | EVQLLESGGGLVQPGGSLRLSCAAS |
| NO: 78 | | GFTFSSYAMSWVRQAPGKGLEWVSA |
| | | ISGSGGSTYYADSVKGRFTISRDNS |
| | | KNTLYLQMNSLRAEDTAVYYCARRE |
| | | WWGESWLFDYWGQGTLVTVSS |
| SEQ ID | DNA VH | GAAGTGCAGTTGCTGGAGTCAGGCG |
| NO: 79 | | GAGGACTGGTGCAGCCCGGAGGATC |
| | | GCTTCGCTTGAGCTGCGCAGCCTCA |
| | | GGCTTTACCTTCTCCTCCTACGCCA |
| | | TGTCCTGGGTCAGACAGGCTCCCGG |
| | | GAAGGGACTGGAATGGGTGTCCGCC |
| | | ATTAGCGGTTCCGGCGGAAGCACTT |
| | | ACTATGCCGACTCTGTGAAGGGCCG |
| | | CTTCACTATCTCCCGGGACAACTCC |
| | | AAGAACACCCTGTATCTCCAAATGA |
| | | ATTCCCTGAGGGCCGAAGATACCGC |
| | | GGTGTACTACTGCGCTAGACGGGAG |
| | | TGGTGGGGAGAAAGCTGGCTGTTCG |
| | | ACTACTGGGGACAGGGCACTCTCGT |
| | | GACTGTGTCCTCC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPLT |
| NO: 56 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPL |
| NO: 59 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPLT |
| NO: 56 | (IMGT) | |
| SEQ ID | VL | DIQMTQSPSSLSASVGDRVTITCRA |
| NO: 61 | | SQSISSYLNWYQQKPGKAPKLLIYA |
| | | ASSLQSGVPSRFSGSGSGTDFTLTI |
| | | SSLQPEDFATYYCQQSYSTPLTFGQ |
| | | GTKVEIK |
| SEQ ID | DNA VL | GACATTCAAATGACTCAGTCCCCGT |
| NO: 62 | | CCTCCCTCTCCGCCTCCGTGGGAGA |
| | | TCGCGTCACGATCACGTGCAGGGCC |
| | | AGCCAGAGCATCTCCAGCTACCTGA |
| | | ACTGGTACCAGCAGAAGCCAGGGAA |
| | | GGCACCGAAGCTCCTGATCTACGCC |
| | | GCTAGCTCGCTGCAGTCCGGCGTCC |
| | | CTTCACGGTTCTCGGGATCGGGCTC |
| | | AGGCACCGACTTCACCCTGACCATT |
| | | AGCAGCCTGCAGCCGGAGGACTTCG |
| | | CGACATACTACTGTCAGCAGTCATA |
| | | CTCCACCCCTCTGACCTTCGGCCAA |
| | | GGGACCAAAGTGGAGATCAAG |
| SEQ ID | Linker | GGGGSGGGGSGGGGSGGGGS |
| NO: 63 | | |
| SEQ ID | scFv | EVQLLESGGGLVQPGGSLRLSCAAS |
| NO: 80 | (VH- | GFTFSSYAMSWVRQAPGKGLEWVSA |
| | linker- | ISGSGGSTYYADSVKGRFTISRDNS |
| | VL) | KNTLYLQMNSLRAEDTAVYYCARRE |
| | | WWGESWLFDYWGQGTLVTVSSGGGG |
| | | SGGGGSGGGGSGGGGSDIQMTQSPS |
| | | SLSASVGDRVTITCRASQSISSYLN |
| | | WYQQKPGKAPKLLIYAASSLQSGVP |
| | | SRFSGSGSGTDFTLTISSLQPEDFA |
| | | TYYCQQSYSTPLTFGQGTKVEIK |
| SEQ ID | DNA | GAAGTGCAGTTGCTGGAGTCAGGCG |
| NO: 81 | scFv | GAGGACTGGTGCAGCCCGGAGGATC |
| | | GCTTCGCTTGAGCTGCGCAGCCTCA |
| | | GGCTTTACCTTCTCCTCCTACGCCA |
| | | TGTCCTGGGTCAGACAGGCTCCCGG |
| | | GAAGGGACTGGAATGGGTGTCCGCC |
| | | ATTAGCGGTTCCGGCGGAAGCACTT |
| | | ACTATGCCGACTCTGTGAAGGGCCG |
| | | CTTCACTATCTCCCGGGACAACTCC |
| | | AAGAACACCCTGTATCTCCAAATGA |
| | | ATTCCCTGAGGGCCGAAGATACCGC |
| | | GGTGTACTACTGCGCTAGACGGGAG |
| | | TGGTGGGGAGAAAGCTGGCTGTTCG |
| | | ACTACTGGGGACAGGGCACTCTCGT |
| | | GACTGTGTCCTCCGGTGGTGGTGGA |
| | | TCGGGGGGTGGTGGTTCGGGCGGAG |
| | | GAGGATCTGGAGGAGGAGGGTCGGA |
| | | CATTCAAATGACTCAGTCCCCGTCC |
| | | TCCCTCTCCGCCTCCGTGGGAGATC |
| | | GCGTCACGATCACGTGCAGGGCCAG |
| | | CCAGAGCATCTCCAGCTACCTGAAC |
| | | TGGTACCAGCAGAAGCCAGGGAAGG |
| | | CACCGAAGCTCCTGATCTACGCCGC |
| | | TAGCTCGCTGCAGTCCGGCGTCCCT |
| | | TCACGGTTCTCGGGATCGGGCTCAG |
| | | GCACCGACTTCACCCTGACCATTAG |
| | | CAGCCTGCAGCCGGAGGACTTCGCG |
| | | ACATACTACTGTCAGCAGTCATACT |
| | | CCACCCCTCTGACCTTCGGCCAAGG |
| | | GACCAAAGTGGAGATCAAG |
| SEQ ID | Full | EVQLLESGGGLVQPGGSLRLSCAAS |
| NO: 82 | CAR | GFTFSSYAMSWVRQAPGKGLEWVSA |
| | amino | ISGSGGSTYYADSVKGRFTISRDNS |
| | acid | KNTLYLQMNSLRAEDTAVYYCARRE |
| | sequence | WWGESWLFDYWGQGTLVTVSSGGGG |
| | | SGGGGSGGGGSGGGGSDIQMTQSPS |
| | | SLSASVGDRVTITCRASQSISSYLN |
| | | WYQQKPGKAPKLLIYAASSLQSGVP |
| | | SRFSGSGSGTDFTLTISSLQPEDFA |
| | | TYYCQQSYSTPLTFGQGTKVEIKTT |
| | | TPAPRPPTPAPTIASQPLSLRPEAC |
| | | RPAAGGAVHTRGLDFACDIYIWAPL |
| | | AGTCGVLLLSLVITLYCKRGRKKLL |
| | | YIFKQPFMRPVQTTQEEDGCSCRFP |
| | | EEEEGGCELRVKFSRSADAPAYQQG |
| | | QNQLYNELNLGRREEYDVLDKRRGR |
| | | DPEMGGKPRRKNPQEGLYNELQKDK |
| | | MAEAYSEIGMKGERRRGKGHDGLYQ |
| | | GLSTATKDTYDALHMQALPPR |
| SEQ ID | Full | GAAGTGCAGTTGCTGGAGTCAGGCG |
| NO: 83 | CAR | GAGGACTGGTGCAGCCCGGAGGATC |
| | DNA | GCTTCGCTTGAGCTGCGCAGCCTCA |
| | sequence | GGCTTTACCTTCTCCTCCTACGCCA |
| | | TGTCCTGGGTCAGACAGGCTCCCGG |
| | | GAAGGGACTGGAATGGGTGTCCGCC |
| | | ATTAGCGGTTCCGGCGGAAGCACTT |
| | | ACTATGCCGACTCTGTGAAGGGCCG |
| | | CTTCACTATCTCCCGGGACAACTCC |
| | | AAGAACACCCTGTATCTCCAAATGA |
| | | ATTCCCTGAGGGCCGAAGATACCGC |
| | | GGTGTACTACTGCGCTAGACGGGAG |
| | | TGGTGGGGAGAAAGCTGGCTGTTCG |
| | | ACTACTGGGGACAGGGCACTCTCGT |
| | | GACTGTGTCCTCCGGTGGTGGTGGA |
| | | TCGGGGGGTGGTGGTTCGGGCGGAG |
| | | GAGGATCTGGAGGAGGAGGGTCGGA |
| | | CATTCAAATGACTCAGTCCCCGTCC |
| | | TCCCTCTCCGCCTCCGTGGGAGATC |
| | | GCGTCACGATCACGTGCAGGGCCAG |
| | | CCAGAGCATCTCCAGCTACCTGAAC |
| | | TGGTACCAGCAGAAGCCAGGGAAGG |
| | | CACCGAAGCTCCTGATCTACGCCGC |
| | | TAGCTCGCTGCAGTCCGGCGTCCCT |
| | | TCACGGTTCTCGGGATCGGGCTCAG |
| | | GCACCGACTTCACCCTGACCATTAG |
| | | CAGCCTGCAGCCGGAGGACTTCGCG |
| | | ACATACTACTGTCAGCAGTCATACT |
| | | CCACCCCTCTGACCTTCGGCCAAGG |
| | | GACCAAAGTGGAGATCAAGACCACT |
| | | ACCCCAGCACCGAGGCCACCCACCC |
| | | CGGCTCCTACCATCGCCTCCCAGCC |
| | | TCTGTCCCTGCGTCCGGAGGCATGT |
| | | AGACCCGCAGCTGGTGGGGCCGTGC |
| | | ATACCCGGGGTCTTGACTTCGCCTG |
| | | CGATATCTACATTTGGGCCCCTCTG |
| | | GCTGGTACTTGCGGGGTCCTGCTGC |
| | | TTTCACTCGTGATCACTCTTTACTG |
| | | TAAGCGCGGTCGGAAGAAGCTGCTG |
| | | TACATCTTTAAGCAACCCTTCATGA |
| | | GGCCTGTGCAGACTACTCAAGAGGA |
| | | GGACGGCTGTTCATGCCGGTTCCCA |
| | | GAGGAGGAGGAAGGCGGCTGCGAAC |
| | | TGCGCGTGAAATTCAGCCGCAGCGC |
| | | AGATGCTCCAGCCTACCAGCAGGGG |
| | | CAGAACCAGCTCTACAACGAACTCA |
| | | ATCTTGGTCGGAGAGAGGAGTACGA |
| | | CGTGCTGGACAAGCGGAGAGGACGG |
| | | GACCCAGAAATGGGCGGGAAGCCGC |
| | | GCAGAAAGAATCCCCAAGAGGGCCT |
| | | GTACAACGAGCTCCAAAAGGATAAG |
| | | ATGGCAGAAGCCTATAGCGAGATTG |
| | | GTATGAAAGGGGAACGCAGAAGAGG |
| | | CAAAGGCCACGACGGACTGTACCAG |
| | | GGACTCAGCACCGCCACCAAGGACA |
| | | CCTATGACGCTCTTCACATGCAGGC |
| | | CCTGCCGCCTCGG |

TABLE 4
Kabat CDRs of exemplary PALLAS-derived anti-BCMA molecules

| Kabat | HCDR1 | HCDR2 | HCDR3 | LCDR1 | LCDR2 | LCDR3 |
|---|---|---|---|---|---|---|
| R1B6 | SYAMS | AISGSGGSTY | REWVPYDVS | RASQSISS | AASSL | QQSYSTP |
| | (SEQ ID | YADSVKG | WYFDY (SEQ | YLN (SEQ | QS | LT (SEQ |
| | NO: 44) | (SEQ ID NO: | ID NO: 46) | ID NO: 54) | (SEQ ID | ID NO: |
| | | 45) | | | NO: 55) | 56) |
| R1F2 | SYAMS | AISGSGGSTY | REWWYDD | RASQSISS | AASSL | QQSYSTP |
| | (SEQ ID | YADSVKG | WYLDY (SEQ | YLN (SEQ | QS | LT |
| | NO: 44) | (SEQ ID NO: | ID NO: 68) | ID NO: 54) | (SEQ ID | (SEQ |
| | | 45) | | | NO: 55) | ID NO: |
| | | | | | | 56) |
| R1G5 | SYAMS | AISGSGGSTY | REWWGESW | RASQSISS | AASSL | QQSYSTP |
| | (SEQ ID | YADSVKG | LFDY (SEQ | YLN (SEQ | QS | LT (SEQ |
| | NO: 44) | (SEQ ID NO: | ID NO: 76) | ID NO: 54) | (SEQ ID | ID NO: |
| | | 45) | | | NO: 55) | 56) |
| Consensus | SYAMS | AISGSGGSTY | REWX₁X₂X₃X₄ | RASQSISS | AASSL | QQSYSTP |
| | (SEQ ID | YADSVKG | X₅X₆WX₇X₈D | YLN (SEQ | QS | LT (SEQ |
| | NO: 44) | (SEQ ID NO: | Y, wherein X₁ | ID NO: 54) | (SEQ ID | ID NO: |
| | | 45) | is absent or V; | | NO: 55) | 56) |
| | | | X₂ is absent or | | | |
| | | | P; X₃ is W or | | | |
| | | | Y; X₄ is G, Y, | | | |
| | | | or D; X₅ is E, | | | |
| | | | D, or V; X₆ is | | | |
| | | | S or D; X₇ is L | | | |
| | | | or Y; and X₈ is | | | |
| | | | For L (SEQ | | | |
| | | | ID NO: 84) | | | |

TABLE 5
Chothia CDRs of exemplary PALLAS-derived anti-BCMA molecules

| Chothia | HCDR1 | HCDR2 | HCDR3 | LCDR1 | LCDR2 | LCDR3 |
|---|---|---|---|---|---|---|
| R1B6 | GFTFSSY | SGSGGS (SEQ | REWVPYDVS | SQSISSY | AAS | SYSTPL |
| | (SEQ ID | ID NO: 48) | WYFDY (SEQ | (SEQ ID | (SEQ ID | (SEQ ID |
| | NO: 47) | | ID NO: 46) | NO: 57) | NO: 58) | NO: 59) |
| R1F2 | GFTFSSY | SGSGGS (SEQ | REWWYDD | SQSISSY | AAS | SYSTPL |
| | (SEQ ID | ID NO: 48) | WYLDY (SEQ | (SEQ ID | (SEQ ID | (SEQ ID |
| | NO: 47) | | ID NO: 68) | NO: 57) | NO: 58) | NO: 59) |
| R1G5 | GFTFSSY | SGSGGS (SEQ | REWWGESW | SQSISSY | AAS | SYSTPL |
| | (SEQ ID | ID NO: 48) | LFDY (SEQ | (SEQ ID | (SEQ ID | (SEQ ID |
| | NO: 47) | | ID NO: 76) | NO: 57) | NO: 58) | NO: 59) |
| Consensus | GFTFSSY | SGSGGS | REWX₁X₂X₃X₄ | SQSISSY | AAS | SYSTPL |
| | (SEQ ID | (SEQ | X₅X₆WX₇X₈D | (SEQ ID | (SEQ ID | (SEQ ID |
| | NO: 47) | ID NO: 48) | Y, wherein X₁ | NO: 57) | NO: 58) | NO: 59) |
| | | | is absent or V; | | | |
| | | | X₂ is absent or | | | |
| | | | P; X₃ is W or | | | |
| | | | Y; X₄ is G, Y, | | | |
| | | | or D; X₅ is E, | | | |
| | | | D, or V; X₆ is | | | |
| | | | S or D; X₇ is L | | | |
| | | | or Y; and X₈ is | | | |
| | | | For L (SEQ | | | |
| | | | ID NO: 84) | | | |

TABLE 6
IMGT CDRs of exemplary PALLAS-derived anti-BCMA molecules

| IMGT | HCDR1 | HCDR2 | HCDR3 | LCDR1 | LCDR2 | LCDR3 |
|---|---|---|---|---|---|---|
| R1B6 | GFTFSSYA | ISGSGGST | ARREWVPY | QSISSY | AAS | QQSYSTP |
| | (SEQ ID | (SEQ ID NO: | DVSWYFDY | (SEQ ID | (SEQ ID | LT (SEQ |
| | NO: 49) | 50) | (SEQ ID NO: | NO: 60) | NO: 58) | ID NO: |
| | | | 51) | | | 56) |
| R1F2 | GFTFSSYA | ISGSGGST | ARREWWYD | QSISSY | AAS | QQSYSTP |
| | (SEQ ID | (SEQ ID NO: | DWYLDY | (SEQ ID | (SEQ ID | LT (SEQ |
| | NO: 49) | 50) | (SEQ ID NO: | NO: 60) | NO: 58) | ID NO: |
| | | | 69) | | | 56) |
| R1G5 | GFTFSSYA | ISGSGGST | ARREWWGE | QSISSY | AAS | QQSYSTP |
| | (SEQ ID | (SEQ ID NO: | SWLFDY | (SEQ ID | (SEQ ID | LT (SEQ |
| | NO: 49) | 50) | (SEQ ID NO: | NO: 60) | NO: 58) | ID NO: |
| | | | 77) | | | 56) |
| Consensus | GFTFSSYA | ISGSGGST | ARREWX₁X₂ | QSISSY | AAS | QQSYSTP |
| | (SEQ ID | (SEQ ID NO: | X₃X₄X₅X₆WX₇ | (SEQ ID | (SEQ ID | LT (SEQ |
| | NO: 49) | 50) | X₈DY, wherein | NO: 60) | NO: 58) | ID NO: |
| | | | X₁ is absent or | | | 56) |
| | | | V; X₂ is absent | | | |
| | | | or P; X₃ is W | | | |
| | | | or Y; X₄ is G, | | | |
| | | | Y, or D; X₅ is | | | |
| | | | E, D, or V; X₆ | | | |
| | | | is S or D; X₇ is | | | |
| | | | L or Y; and X₈ | | | |
| | | | is F or L (SEQ | | | |
| | | | ID NO: 85) | | | |

TABLE 7
Amino acid and nucleic acid sequences of
exemplary B cell-derived anti-BCMA
molecules

| SEQ ID | Name/ | |
|---|---|---|
| NO | Description | Sequence |
| PI61 | | |
| SEQ ID | HCDR1 | SYGMH |
| NO: 86 | (Kabat) | |
| SEQ ID | HCDR2 | VISYDGSNKYYADSVKG |
| NO: 87 | (Kabat) | |
| SEQ ID | HCDR3 | SGYALHDDYYGLDV |
| NO: 88 | (Kabat) | |
| SEQ ID | HCDR1 | GFTFSSY |
| NO: 47 | (Chothia) | |
| SEQ ID | HCDR2 | SYDGSN |
| NO: 89 | (Chothia) | |
| SEQ ID | HCDR3 | SGYALHDDYYGLDV |
| NO: 88 | (Chothia) | |
| SEQ ID | HCDR1 | GFTFSSYG |
| NO: 90 | (IMGT) | |
| SEQ ID | HCDR2 | ISYDGSNK |
| NO: 91 | (IMGT) | |
| SEQ ID | HCDR3 | GGSGYALHDDYYGLDV |
| NO: 92 | (IMGT) | |
| SEQ ID | VH | QVQLQESGGGVVQPGRSLRLSCAAS |
| NO: 93 | | GFTFSSYGMHWVRQAPGKGLEWVAV |
| | | ISYDGSNKYYADSVKGRFTISRDNS |
| | | KNTLYLQMNSLRAEDTAVYYCGGSG |
| | | YALHDDYYGLDVWGQGTLVTVSS |
| SEQ ID | DNA VH | CAAGTGCAGCTGCAGGAATCCGGTG |
| NO: 94 | | GCGGAGTCGTGCAGCCTGGAAGGAG |
| | | CCTGAGACTCTCATGCGCCGCGTCA |
| | | GGGTTCACCTTTTCCTCCTACGGGA |
| | | TGCATTGGGTCAGACAGGCCCCCGG |
| | | AAAGGGACTCGAATGGGTGGCTGTG |
| | | ATCAGCTACGACGGCTCCAACAAGT |
| | | ACTACGCCGACTCCGTGAAAGGCCG |
| | | GTTCACTATCTCCCGGGACAACTCC |
| | | AAGAACACGCTGTATCTGCAAATGA |
| | | ATTCACTGCGCGCGGAGGATACCGC |
| | | TGTGTACTACTGCGGTGGCTCCGGT |
| | | TACGCCCTGCACGATGACTATTACG |
| | | GCCTTGACGTCTGGGGCCAGGGAAC |
| | | CCTCGTGACTGTGTCCAGC |
| SEQ ID | LCDR1 | TGTSSDVGGYNYVS |
| NO: 95 | (Kabat) | |
| SEQ ID | LCDR2 | DVSNRPS |
| NO: 96 | (Kabat) | |
| SEQ ID | LCDR3 | SSYTSSSTLYV |
| NO: 97 | (Kabat) | |
| SEQ ID | LCDR1 | TSSDVGGYNY |
| NO: 98 | (Chothia) | |
| SEQ ID | LCDR2 | DVS |
| NO: 99 | (Chothia) | |
| SEQ ID | LCDR3 | YTSSSTLY |
| NO: 100 | (Chothia) | |
| SEQ ID | LCDR1 | SSDVGGYNY |
| NO: 101 | (IMGT) | |
| SEQ ID | LCDR2 | DVS |
| NO: 99 | (IMGT) | |
| SEQ ID | LCDR3 | SSYTSSSTLYV |
| NO: 97 | (IMGT) | |
| SEQ ID | VL | QSALTQPASVSGSPGQSITISCTGT |
| NO: 102 | | SSDVGGYNYVSWYQQHPGKAPKLMI |
| | | YDVSNRPSGVSNRFSGSKSGNTASL |
| | | TISGLQAEDEADYYCSSYTSSSTLY |
| | | VFGSGTKVTVL |
| SEQ ID | DNA VL | CAGAGCGCACTGACTCAGCCGGCAT |
| NO: 103 | | CCGTGTCCGGTAGCCCCGGACAGTC |
| | | GATTACCATCTCCTGTACCGGCACC |
| | | TCCTCCGACGTGGGAGGGTACAACT |
| | | ACGTGTCGTGGTACCAGCAGCACCC |
| | | AGGAAAGGCCCCTAAGTTGATGATC |
| | | TACGATGTGTCAAACCGCCCGTCTG |
| | | GAGTCTCCAACCGGTTCTCCGGCTC |
| | | CAAGTCCGGCAACACCGCCAGCCTG |
| | | ACCATTAGCGGGCTGCAAGCCGAGG |
| | | ATGAGGCCGACTACTACTGCTCGAG |
| | | CTACACATCCTCGAGCACCCTCTAC |
| | | GTGTTCGGCTCGGGGACTAAGGTCA |
| | | CCGTGCTG |
| SEQ ID | Linker | GGGGSGGGGSGGGGS |
| NO: 104 | | |
| SEQ ID | scFv (VH- | QVQLQESGGGVVQPGRSLRLSCAAS |
| NO: 105 | linker-VL) | GFTFSSYGMHWVRQAPGKGLEWVAV |
| | | ISYDGSNKYYADSVKGRFTISRDNS |
| | | KNTLYLQMNSLRAEDTAVYYCGGSG |
| | | YALHDDYYGLDVWGQGTLVTVSSGG |
| | | GGSGGGGSGGGGSQSALTQPASVSG |
| | | SPGQSITISCTGTSSDVGGYNYVSW |
| | | YQQHPGKAPKLMIYDVSNRPSGVSN |
| | | RFSGSKSGNTASLTISGLQAEDEAD |
| | | YYCSSYTSSSTLYVFGSGTKVTVL |
| SEQ ID | DNA scFv | CAAGTGCAGCTGCAGGAATCCGGTG |
| NO: 106 | | GCGGAGTCGTGCAGCCTGGAAGGAG |
| | | CCTGAGACTCTCATGCGCCGCGTCA |
| | | GGGTTCACCTTTTCCTCCTACGGGA |
| | | TGCATTGGGTCAGACAGGCCCCCGG |
| | | AAAGGGACTCGAATGGGTGGCTGTG |
| | | ATCAGCTACGACGGCTCCAACAAGT |
| | | ACTACGCCGACTCCGTGAAAGGCCG |
| | | GTTCACTATCTCCCGGGACAACTCC |
| | | AAGAACACGCTGTATCTGCAAATGA |
| | | ATTCACTGCGCGCGGAGGATACCGC |
| | | TGTGTACTACTGCGGTGGCTCCGGT |
| | | TACGCCCTGCACGATGACTATTACG |
| | | GCCTTGACGTCTGGGGCCAGGGAAC |
| | | CCTCGTGACTGTGTCCAGCGGTGGA |
| | | GGAGGTTCGGGCGGAGGAGGATCAG |
| | | GAGGGGGTGGATCGCAGAGCGCACT |
| | | GACTCAGCCGGCATCCGTGTCCGGT |
| | | AGCCCCGGACAGTCGATTACCATCT |
| | | CCTGTACCGGCACCTCCTCCGACGT |
| | | GGGAGGGTACAACTACGTGTCGTGG |
| | | TACCAGCAGCACCCAGGAAAGGCCC |
| | | CTAAGTTGATGATCTACGATGTGTC |
| | | AAACCGCCCGTCTGGAGTCTCCAAC |
| | | CGGTTCTCCGGCTCCAAGTCCGGCA |
| | | ACACCGCCAGCCTGACCATTAGCGG |
| | | GCTGCAAGCCGAGGATGAGGCCGAC |
| | | TACTACTGCTCGAGCTACACATCCT |
| | | CGAGCACCCTCTACGTGTTCGGCTC |
| | | GGGGACTAAGGTCACCGTGCTG |
| SEQ ID | Full CAR | QVQLQESGGGVVQPGRSLRLSCAAS |
| NO: 107 | amino acid | GFTFSSYGMHWVRQAPGKGLEWVAV |
| | sequence | ISYDGSNKYYADSVKGRFTISRDNS |
| | | KNTLYLQMNSLRAEDTAVYYCGGSG |
| | | YALHDDYYGLDVWGQGTLVTVSSGG |
| | | GGSGGGGSGGGGSQSALTQPASVSG |
| | | SPGQSITISCTGTSSDVGGYNYVSW |
| | | YQQHPGKAPKLMIYDVSNRPSGVSN |
| | | RFSGSKSGNTASLTISGLQAEDEAD |
| | | YYCSSYTSSSTLYVFGSGTKVTVLT |
| | | TTPAPRPPTPAPTIASQPLSLRPEA |
| | | CRPAAGGAVHTRGLDFACDIYIWAP |
| | | LAGTCGVLLLSLVITLYCKRGRKKL |
| | | LYIFKQPFMRPVQTTQEEDGCSCRF |
| | | PEEEEGGCELRVKFSRSADAPAYQQ |
| | | GQNQLYNELNLGRREEYDVLDKRRG |
| | | RDPEMGGKPRRKNPQEGLYNELQKD |
| | | KMAEAYSEIGMKGERRRGKGHDGLY |
| | | QGLSTATKDTYDALHMQALPPR |
| SEQ ID | Full CAR | CAAGTGCAGCTGCAGGAATCCGGTG |
| NO: 108 | DNA | GCGGAGTCGTGCAGCCTGGAAGGAG |
| | sequence | CCTGAGACTCTCATGCGCCGCGTCA |
| | | GGGTTCACCTTTTCCTCCTACGGGA |
| | | TGCATTGGGTCAGACAGGCCCCCGG |
| | | AAAGGGACTCGAATGGGTGGCTGTG |
| | | ATCAGCTACGACGGCTCCAACAAGT |
| | | ACTACGCCGACTCCGTGAAAGGCCG |
| | | GTTCACTATCTCCCGGGACAACTCC |
| | | AAGAACACGCTGTATCTGCAAATGA |
| | | ATTCACTGCGCGCGGAGGATACCGC |
| | | TGTGTACTACTGCGGTGGCTCCGGT |
| | | TACGCCCTGCACGATGACTATTACG |
| | | GCCTTGACGTCTGGGGCCAGGGAAC |
| | | CCTCGTGACTGTGTCCAGCGGTGGA |
| | | GGAGGTTCGGGCGGAGGAGGATCAG |
| | | GAGGGGGTGGATCGCAGAGCGCACT |
| | | GACTCAGCCGGCATCCGTGTCCGGT |
| | | AGCCCCGGACAGTCGATTACCATCT |
| | | CCTGTACCGGCACCTCCTCCGACGT |
| | | GGGAGGGTACAACTACGTGTCGTGG |
| | | TACCAGCAGCACCCAGGAAAGGCCC |
| | | CTAAGTTGATGATCTACGATGTGTC |
| | | AAACCGCCCGTCTGGAGTCTCCAAC |
| | | CGGTTCTCCGGCTCCAAGTCCGGCA |
| | | ACACCGCCAGCCTGACCATTAGCGG |
| | | GCTGCAAGCCGAGGATGAGGCCGAC |
| | | TACTACTGCTCGAGCTACACATCCT |
| | | CGAGCACCCTCTACGTGTTCGGCTC |
| | | GGGGACTAAGGTCACCGTGCTGACC |
| | | ACTACCCCAGCACCGAGGCCACCCA |
| | | CCCCGGCTCCTACCATCGCCTCCCA |
| | | GCCTCTGTCCCTGCGTCCGGAGGCA |
| | | TGTAGACCCGCAGCTGGTGGGGCCG |
| | | TGCATACCCGGGGTCTTGACTTCGC |
| | | CTGCGATATCTACATTTGGGCCCCT |
| | | CTGGCTGGTACTTGCGGGGTCCTGC |
| | | TGCTTTCACTCGTGATCACTCTTTA |
| | | CTGTAAGCGCGGTCGGAAGAAGCTG |
| | | CTGTACATCTTTAAGCAACCCTTCA |
| | | TGAGGCCTGTGCAGACTACTCAAGA |
| | | GGAGGACGGCTGTTCATGCCGGTTC |
| | | CCAGAGGAGGAGGAAGGCGGCTGCG |
| | | AACTGCGCGTGAAATTCAGCCGCAG |
| | | CGCAGATGCTCCAGCCTACCAGCAG |
| | | GGGCAGAACCAGCTCTACAACGAAC |
| | | TCAATCTTGGTCGGAGAGAGGAGTA |
| | | CGACGTGCTGGACAAGCGGAGAGGA |
| | | CGGGACCCAGAAATGGGCGGGAAGC |
| | | CGCGCAGAAAGAATCCCCAAGAGGG |
| | | CCTGTACAACGAGCTCCAAAAGGAT |
| | | AAGATGGCAGAAGCCTATAGCGAGA |
| | | TTGGTATGAAAGGGGAACGCAGAAG |
| | | AGGCAAAGGCCACGACGGACTGTAC |
| | | CAGGGACTCAGCACCGCCACCAAGG |
| | | ACACCTATGACGCTCTTCACATGCA |
| | | GGCCCTGCCGCCTCGG |
| B61-02 | | |
| SEQ ID | HCDR1 | SYGMH |
| NO: 86 | (Kabat) | |
| SEQ ID | HCDR2 | VISYKGSNKYYADSVKG |
| NO: 109 | (Kabat) | |
| SEQ ID | HCDR3 | SGYALHDDYYGLDV |
| NO: 88 | (Kabat) | |
| SEQ ID | HCDR1 | GFTFSSY |
| NO: 47 | (Chothia) | |
| SEQ ID | HCDR2 | SYKGSN |
| NO: 110 | (Chothia) | |
| SEQ ID | HCDR3 | SGYALHDDYYGLDV |
| NO: 88 | (Chothia) | |
| SEQ ID | HCDR1 | GFTFSSYG |
| NO: 90 | (IMGT) | |
| SEQ ID | HCDR2 | ISYKGSNK |
| NO: 111 | (IMGT) | |
| SEQ ID | HCDR3 | GGSGYALHDDYYGLDV |
| NO: 92 | (IMGT) | |
| SEQ ID | VH | QVQLVESGGGVVQPGRSLRLSCAAS |
| NO: 112 | | GFTFSSYGMHWVRQAPGKGLEWVAV |
| | | ISYKGSNKYYADSVKGRFTISRDNS |
| | | KNTLYLQMNSLRAEDTAVYYCGGSG |
| | | YALHDDYYGLDVWGQGTLVTVSS |
| SEQ ID | DNA VH | CAAGTGCAGCTTGTCGAATCGGGAG |
| NO: 113 | | GCGGAGTGGTGCAGCCTGGACGATC |
| | | GCTCCGGCTCTCATGTGCCGCGAGC |
| | | GGATTCACCTTCTCGAGCTACGGCA |
| | | TGCACTGGGTCAGACAAGCCCCAGG |
| | | AAAGGGCCTGGAATGGGTGGCTGTC |
| | | ATCTCGTACAAGGGCTCAAACAAGT |
| | | ACTACGCCGACTCCGTGAAGGGCCG |
| | | GTTCACCATCTCCCGCGATAACTCC |
| | | AAGAATACCCTCTATCTGCAAATGA |
| | | ACAGCCTGAGGGCCGAGGATACTGC |
| | | AGTGTACTACTGCGGGGGTTCAGGC |
| | | TACGCGCTGCACGACGACTACTACG |
| | | GATTGGACGTCTGGGGCCAAGGAAC |
| | | TCTTGTGACCGTGTCCTCT |
| SEQ ID | LCDR1 | TGTSSDVGGYNYVS |
| NO: 95 | (Kabat) | |
| SEQ ID | LCDR2 | EVSNRLR |
| NO: 114 | (Kabat) | |
| SEQ ID | LCDR3 | SSYTSSSALYV |
| NO: 115 | (Kabat) | |
| SEQ ID | LCDR1 | TSSDVGGYNY |
| NO: 98 | (Chothia) | |
| SEQ ID | LCDR2 | EVS |
| NO: 116 | (Chothia) | |
| SEQ ID | LCDR3 | YTSSSALY |
| NO: 117 | (Chothia) | |
| SEQ ID | LCDR1 | SSDVGGYNY |
| NO: 101 | (IMGT) | |
| SEQ ID | LCDR2 | EVS |
| NO: 116 | (IMGT) | |
| SEQ ID | LCDR3 | SSYTSSSALYV |
| NO: 115 | (IMGT) | |
| SEQ ID | VL | QSALTQPASVSGSPGQSITISCTGT |
| NO: 118 | | SSDVGGYNYVSWYQQHPGKAPKLMI |
| | | YEVSNRLRGVSNRFSGSKSGNTASL |
| | | TISGLQAEDEADYYCSSYTSSSALY |
| | | VFGSGTKVTVL |
| SEQ ID | DNA VL | CAGAGCGCGCTGACTCAGCCTGCCT |
| NO: 119 | | CCGTGAGCGGTTCGCCGGGACAGTC |
| | | CATTACCATTTCGTGCACCGGGACC |
| | | TCCTCCGACGTGGGAGGCTACAACT |
| | | ACGTGTCCTGGTACCAGCAGCATCC |
| | | CGGAAAGGCCCCGAAGCTGATGATC |
| | | TACGAAGTGTCGAACAGACTGCGGG |
| | | GAGTCTCCAACCGCTTTTCCGGGTC |
| | | CAAGTCCGGCAACACCGCCAGCCTG |
| | | ACCATCAGCGGGCTCCAGGCAGAAG |
| | | ATGAGGCTGACTATTACTGCTCCTC |
| | | CTACACGTCAAGCTCCGCCCTCTAC |
| | | GTGTTCGGGTCCGGGACCAAAGTCA |
| | | CTGTGCTG |
| SEQ ID | Linker | GGGGSGGGGGGGGSGGGGS |
| NO: 63 | | |
| SEQ ID | scFv | QVQLVESGGGVVQPGRSLRLSCAAS |
| NO: 120 | (VH- | GFTFSSYGMHWVRQAPGKGLEWVAV |
| | linker- | ISYKGSNKYYADSVKGRFTISRDNS |
| | VL) | KNTLYLQMNSLRAEDTAVYYCGGSG |
| | | YALHDDYYGLDVWGQGTLVTVSSGG |
| | | GGSGGGGSGGGGSGGGGSQSALTQP |
| | | ASVSGSPGQSITISCTGTSSDVGGY |
| | | NYVSWYQQHPGKAPKLMIYEVSNRL |
| | | RGVSNRFSGSKSGNTASLTISGLQA |
| | | EDEADYYCSSYTSSSALYVFGSGTK |
| | | VTVL |
| SEQ ID | DNA scFv | CAAGTGCAGCTTGTCGAATCGGGAG |
| NO: 121 | | GCGGAGTGGTGCAGCCTGGACGATC |
| | | GCTCCGGCTCTCATGTGCCGCGAGC |
| | | GGATTCACCTTCTCGAGCTACGGCA |
| | | TGCACTGGGTCAGACAAGCCCCAGG |
| | | AAAGGGCCTGGAATGGGTGGCTGTC |
| | | ATCTCGTACAAGGGCTCAAACAAGT |
| | | ACTACGCCGACTCCGTGAAGGGCCG |
| | | GTTCACCATCTCCCGCGATAACTCC |
| | | AAGAATACCCTCTATCTGCAAATGA |
| | | ACAGCCTGAGGGCCGAGGATACTGC |
| | | AGTGTACTACTGCGGGGGTTCAGGC |
| | | TACGCGCTGCACGACGACTACTACG |
| | | GATTGGACGTCTGGGGCCAAGGAAC |
| | | TCTTGTGACCGTGTCCTCTGGTGGA |
| | | GGCGGATCAGGGGGTGGCGGATCTG |
| | | GGGGTGGTGGTTCCGGGGGAGGAGG |
| | | ATCGCAGAGCGCGCTGACTCAGCCT |
| | | GCCTCCGTGAGCGGTTCGCCGGGAC |
| | | AGTCCATTACCATTTCGTGCACCGG |
| | | GACCTCCTCCGACGTGGGAGGCTAC |
| | | AACTACGTGTCCTGGTACCAGCAGC |
| | | ATCCCGGAAAGGCCCCGAAGCTGAT |
| | | GATCTACGAAGTGTCGAACAGACTG |
| | | CGGGGAGTCTCCAACCGCTTTTCCG |
| | | GGTCCAAGTCCGGCAACACCGCCAG |
| | | CCTGACCATCAGCGGGCTCCAGGCA |
| | | GAAGATGAGGCTGACTATTACTGCT |
| | | CCTCCTACACGTCAAGCTCCGCCCT |
| | | CTACGTGTTCGGGTCCGGGACCAAA |
| | | GTCACTGTGCTG |
| SEQ ID | Full CAR | QVQLVESGGGVVQPGRSLRLSCAAS |
| NO: 122 | amino | GFTFSSYGMHWVRQAPGKGLEWVAV |
| | acid | ISYKGSNKYYADSVKGRFTISRDNS |
| | sequence | KNTLYLQMNSLRAEDTAVYYCGGSG |
| | | YALHDDYYGLDVWGQGTLVTVSSGG |
| | | GGSGGGGSGGGGSGGGGSQSALTQP |
| | | ASVSGSPGQSITISCTGTSSDVGGY |
| | | NYVSWYQQHPGKAPKLMIYEVSNRL |
| | | RGVSNRFSGSKSGNTASLTISGLQA |
| | | EDEADYYCSSYTSSSALYVFGSGTK |
| | | VTVLTTTPAPRPPTPAPTIASQPLS |
| | | LRPEACRPAAGGAVHTRGLDFACDI |
| | | YIWAPLAGTCGVLLLSLVITLYCKR |
| | | GRKKLLYIFKQPFMRPVQTTQEEDG |
| | | CSCRFPEEEEGGCELRVKFSRSADA |
| | | PAYQQGQNQLYNELNLGRREEYDVL |
| | | DKRRGRDPEMGGKPRRKNPQEGLYN |
| | | ELQKDKMAEAYSEIGMKGERRRGKG |
| | | HDGLYQGLSTATKDTYDALHMQALP |
| | | PR |
| SEQ ID | Full | CAAGTGCAGCTTGTCGAATCGGGAG |
| NO: 123 | CAR | GCGGAGTGGTGCAGCCTGGACGATC |
| | DNA | GCTCCGGCTCTCATGTGCCGCGAGC |
| | sequence | GGATTCACCTTCTCGAGCTACGGCA |
| | | TGCACTGGGTCAGACAAGCCCCAGG |
| | | AAAGGGCCTGGAATGGGTGGCTGTC |
| | | ATCTCGTACAAGGGCTCAAACAAGT |
| | | ACTACGCCGACTCCGTGAAGGGCCG |
| | | GTTCACCATCTCCCGCGATAACTCC |
| | | AAGAATACCCTCTATCTGCAAATGA |
| | | ACAGCCTGAGGGCCGAGGATACTGC |
| | | AGTGTACTACTGCGGGGGTTCAGGC |
| | | TACGCGCTGCACGACGACTACTACG |
| | | GATTGGACGTCTGGGGCCAAGGAAC |
| | | TCTTGTGACCGTGTCCTCTGGTGGA |
| | | GGCGGATCAGGGGGTGGCGGATCTG |
| | | GGGGTGGTGGTTCCGGGGGAGGAGG |
| | | ATCGCAGAGCGCGCTGACTCAGCCT |
| | | GCCTCCGTGAGCGGTTCGCCGGGAC |
| | | AGTCCATTACCATTTCGTGCACCGG |
| | | GACCTCCTCCGACGTGGGAGGCTAC |
| | | AACTACGTGTCCTGGTACCAGCAGC |
| | | ATCCCGGAAAGGCCCCGAAGCTGAT |
| | | GATCTACGAAGTGTCGAACAGACTG |
| | | CGGGGAGTCTCCAACCGCTTTTCCG |
| | | GGTCCAAGTCCGGCAACACCGCCAG |
| | | CCTGACCATCAGCGGGCTCCAGGCA |
| | | GAAGATGAGGCTGACTATTACTGCT |
| | | CCTCCTACACGTCAAGCTCCGCCCT |
| | | CTACGTGTTCGGGTCCGGGACCAAA |
| | | GTCACTGTGCTGACCACTACCCCAG |
| | | CACCGAGGCCACCCACCCCGGCTCC |
| | | TACCATCGCCTCCCAGCCTCTGTCC |
| | | CTGCGTCCGGAGGCATGTAGACCCG |
| | | CAGCTGGTGGGGCCGTGCATACCCG |
| | | GGGTCTTGACTTCGCCTGCGATATC |
| | | TACATTTGGGCCCCTCTGGCTGGTA |
| | | CTTGCGGGGTCCTGCTGCTTTCACT |
| | | CGTGATCACTCTTTACTGTAAGCGC |
| | | GGTCGGAAGAAGCTGCTGTACATCT |
| | | TTAAGCAACCCTTCATGAGGCCTGT |
| | | GCAGACTACTCAAGAGGAGGACGGC |
| | | TGTTCATGCCGGTTCCCAGAGGAGG |
| | | AGGAAGGCGGCTGCGAACTGCGCGT |
| | | GAAATTCAGCCGCAGCGCAGATGCT |
| | | CCAGCCTACCAGCAGGGGCAGAACC |
| | | AGCTCTACAACGAACTCAATCTTGG |
| | | TCGGAGAGAGGAGTACGACGTGCTG |
| | | GACAAGCGGAGAGGACGGGACCCAG |
| | | AAATGGGCGGGAAGCCGCGCAGAAA |
| | | GAATCCCCAAGAGGGCCTGTACAAC |
| | | GAGCTCCAAAAGGATAAGATGGCAG |
| | | AAGCCTATAGCGAGATTGGTATGAA |
| | | AGGGGAACGCAGAAGAGGCAAAGGC |
| | | CACGACGGACTGTACCAGGGACTCA |
| | | GCACCGCCACCAAGGACACCTATGA |
| | | CGCTCTTCACATGCAGGCCCTGCCG |
| | | CCTCGG |
| B61-10 | | |
| SEQ ID | HCDR1 | SYGMH |
| NO: 86 | (Kabat) | |
| SEQ ID | HCDR2 | VISYKGSNKYYADSVKG |
| NO: 109 | (Kabat) | |
| SEQ ID | HCDR3 | SGYALHDDYYGLDV |
| NO: 88 | (Kabat) | |
| SEQ ID | HCDR1 | GFTFSSY |
| NO: 47 | (Chothia) | |
| SEQ ID | HCDR2 | SYKGSN |
| NO: 110 | (Chothia) | |
| SEQ ID | HCDR3 | SGYALHDDYYGLDV |
| NO: 88 | (Chothia) | |
| SEQ ID | HCDR1 | GFTFSSYG |
| NO: 90 | (IMGT) | |
| SEQ ID | HCDR2 | ISYKGSNK |
| NO: 111 | (IMGT) | |
| SEQ ID | HCDR3 | GGSGYALHDDYYGLDV |
| NO: 92 | (IMGT) | |
| SEQ ID | VH | QVQLVESGGGVVQPGRSLRLSCAAS |
| NO: 112 | | GFTFSSYGMHWVRQAPGKGLEWVAV |
| | | ISYKGSNKYYADSVKGRFTISRDNS |
| | | KNTLYLQMNSLRAEDTAVYYCGGSG |
| | | YALHDDYYGLDVWGQGTLVTVSS |
| SEQ ID | DNA VH | CAAGTGCAGCTTGTCGAATCGGGAG |
| NO: 113 | | GCGGAGTGGTGCAGCCTGGACGATC |
| | | GCTCCGGCTCTCATGTGCCGCGAGC |
| | | GGATTCACCTTCTCGAGCTACGGCA |
| | | TGCACTGGGTCAGACAAGCCCCAGG |
| | | AAAGGGCCTGGAATGGGTGGCTGTC |
| | | ATCTCGTACAAGGGCTCAAACAAGT |
| | | ACTACGCCGACTCCGTGAAGGGCCG |
| | | GTTCACCATCTCCCGCGATAACTCC |
| | | AAGAATACCCTCTATCTGCAAATGA |
| | | ACAGCCTGAGGGCCGAGGATACTGC |
| | | AGTGTACTACTGCGGGGGTTCAGGC |
| | | TACGCGCTGCACGACGACTACTACG |
| | | GATTGGACGTCTGGGGCCAAGGAAC |
| | | TCTTGTGACCGTGTCCTCT |
| SEQ ID | LCDR1 | TGTSSDVGGYNYVS |
| NO: 95 | (Kabat) | |
| SEQ ID | LCDR2 | EVSNRLR |
| NO: 114 | (Kabat) | |
| SEQ ID | LCDR3 | SSYTSSSTLYV |
| NO: 97 | (Kabat) | |
| SEQ ID | LCDR1 | TSSDVGGYNY |
| NO: 98 | (Chothia) | |
| SEQ ID | LCDR2 | EVS |
| NO: 116 | (Chothia) | |
| SEQ ID | LCDR3 | YTSSSTLY |
| NO: 100 | (Chothia) | |
| SEQ ID | LCDR1 | SSDVGGYNY |
| NO: 101 | (IMGT) | |
| SEQ ID | LCDR2 | EVS |
| NO: 116 | (IMGT) | |
| SEQ ID | LCDR3 | SSYTSSSTLYV |
| NO: 97 | (IMGT) | |
| SEQ ID | VL | QSALTQPASVSGSPGQSITISCTGT |
| NO: 124 | | SSDVGGYNYVSWYQQHPGKAPKLMI |
| | | YEVSNRLRGVSNRFSGSKSGNTASL |
| | | TISGLQAEDEADYYCSSYTSSSTLY |
| | | VFGSGTKVTVL |
| SEQ ID | DNA VL | CAGAGCGCGCTGACTCAGCCTGCCT |
| NO: 125 | | CCGTGAGCGGTTCGCCGGGACAGTC |
| | | CATTACCATTTCGTGCACCGGGACC |
| | | TCCTCCGACGTGGGAGGCTACAACT |
| | | ACGTGTCCTGGTACCAGCAGCATCC |
| | | CGGAAAGGCCCCGAAGCTGATGATC |
| | | TACGAAGTGTCGAACAGACTGCGGG |
| | | GAGTCTCCAACCGCTTTTCCGGGTC |
| | | CAAGTCCGGCAACACCGCCAGCCTG |
| | | ACCATCAGCGGGCTCCAGGCAGAAG |
| | | ATGAGGCTGACTATTACTGCTCCTC |
| | | CTACACGTCAAGCTCCACCCTCTAC |
| | | GTGTTCGGGTCCGGGACCAAAGTCA |
| | | CTGTGCTG |
| SEQ ID | Linker | GGGGSGGGGSGGGGSGGGGS |
| NO: 63 | | |
| SEQ ID | scFv (VH- | QVQLVESGGGVVQPGRSLRLSCAAS |
| NO: 126 | linker-VL) | GFTFSSYGMHWVRQAPGKGLEWVAV |
| | | ISYKGSNKYYADSVKGRFTISRDNS |
| | | KNTLYLQMNSLRAEDTAVYYCGGSG |
| | | YALHDDYYGLDVWGQGTLVTVSSGG |
| | | GGGGGGSGGGGSGGGGSQSALTQPA |
| | | SVSGSPGQSITISCTGTSSDVGGYN |
| | | YVSWYQQHPGKAPKLMIYEVSNRLR |
| | | GVSNRFSGSKSGNTASLTISGLQAE |
| | | DEADYYCSSYTSSSTLYVFGSGTKV |
| | | TVL |
| SEQ ID | DNA scFv | CAAGTGCAGCTTGTCGAATCGGGAG |
| NO: 127 | | GCGGAGTGGTGCAGCCTGGACGATC |
| | | GCTCCGGCTCTCATGTGCCGCGAGC |
| | | GGATTCACCTTCTCGAGCTACGGCA |
| | | TGCACTGGGTCAGACAAGCCCCAGG |
| | | AAAGGGCCTGGAATGGGTGGCTGTC |
| | | ATCTCGTACAAGGGCTCAAACAAGT |
| | | ACTACGCCGACTCCGTGAAGGGCCG |
| | | GTTCACCATCTCCCGCGATAACTCC |
| | | AAGAATACCCTCTATCTGCAAATGA |
| | | ACAGCCTGAGGGCCGAGGATACTGC |
| | | AGTGTACTACTGCGGGGGTTCAGGC |
| | | TACGCGCTGCACGACGACTACTACG |
| | | GATTGGACGTCTGGGGCCAAGGAAC |
| | | TCTTGTGACCGTGTCCTCTGGTGGA |
| | | GGCGGATCAGGGGGTGGCGGATCTG |
| | | GGGGTGGTGGTTCCGGGGGAGGAGG |
| | | ATCGCAGAGCGCGCTGACTCAGCCT |
| | | GCCTCCGTGAGCGGTTCGCCGGGAC |
| | | AGTCCATTACCATTTCGTGCACCGG |
| | | GACCTCCTCCGACGTGGGAGGCTAC |
| | | AACTACGTGTCCTGGTACCAGCAGC |
| | | ATCCCGGAAAGGCCCCGAAGCTGAT |
| | | GATCTACGAAGTGTCGAACAGACTG |
| | | CGGGGAGTCTCCAACCGCTTTTCCG |
| | | GGTCCAAGTCCGGCAACACCGCCAG |
| | | CCTGACCATCAGCGGGCTCCAGGCA |
| | | GAAGATGAGGCTGACTATTACTGCT |
| | | CCTCCTACACGTCAAGCTCCACCCT |
| | | CTACGTGTTCGGGTCCGGGACCAAA |
| | | GTCACTGTGCTG |
| SEQ ID | Full CAR | QVQLVESGGGVVQPGRSLRLSCAAS |
| NO: 128 | amino acid | GFTFSSYGMHWVRQAPGKGLEWVAV |
| | sequence | ISYKGSNKYYADSVKGRFTISRDNS |
| | | KNTLYLQMNSLRAEDTAVYYCGGSG |
| | | YALHDDYYGLDVWGQGTLVTVSSGG |
| | | GGSGGGGSGGGGSGGGGSQSALTQP |
| | | ASVSGSPGQSITISCTGTSSDVGGY |
| | | NYVSWYQQHPGKAPKLMIYEVSNRL |
| | | RGVSNRFSGSKSGNTASLTISGLQA |
| | | EDEADYYCSSYTSSSTLYVFGSGTK |
| | | VTVLTTTPAPRPPTPAPTIASQPLS |
| | | LRPEACRPAAGGAVHTRGLDFACDI |
| | | YIWAPLAGTCGVLLLSLVITLYCKR |
| | | GRKKLLYIFKQPFMRPVQTTQEEDG |
| | | CSCRFPEEEEGGCELRVKFSRSADA |
| | | PAYQQGQNQLYNELNLGRREEYDVL |
| | | DKRRGRDPEMGGKPRRKNPQEGLYN |
| | | ELQKDKMAEAYSEIGMKGERRRGKG |
| | | HDGLYQGLSTATKDTYDALHMQALP |
| | | PR |
| SEQ ID | Full CAR | CAAGTGCAGCTTGTCGAATCGGGAG |
| NO: 129 | DNA | GCGGAGTGGTGCAGCCTGGACGATC |
| | sequence | GCTCCGGCTCTCATGTGCCGCGAGC |
| | | GGATTCACCTTCTCGAGCTACGGCA |
| | | TGCACTGGGTCAGACAAGCCCCAGG |
| | | AAAGGGCCTGGAATGGGTGGCTGTC |
| | | ATCTCGTACAAGGGCTCAAACAAGT |
| | | ACTACGCCGACTCCGTGAAGGGCCG |
| | | GTTCACCATCTCCCGCGATAACTCC |
| | | AAGAATACCCTCTATCTGCAAATGA |
| | | ACAGCCTGAGGGCCGAGGATACTGC |
| | | AGTGTACTACTGCGGGGGTTCAGGC |
| | | TACGCGCTGCACGACGACTACTACG |
| | | GATTGGACGTCTGGGGCCAAGGAAC |
| | | TCTTGTGACCGTGTCCTCTGGTGGA |
| | | GGCGGATCAGGGGGTGGCGGATCTG |
| | | GGGGTGGTGGTTCCGGGGGAGGAGG |
| | | ATCGCAGAGCGCGCTGACTCAGCCT |
| | | GCCTCCGTGAGCGGTTCGCCGGGAC |
| | | AGTCCATTACCATTTCGTGCACCGG |
| | | GACCTCCTCCGACGTGGGAGGCTAC |
| | | AACTACGTGTCCTGGTACCAGCAGC |
| | | ATCCCGGAAAGGCCCCGAAGCTGAT |
| | | GATCTACGAAGTGTCGAACAGACTG |
| | | CGGGGAGTCTCCAACCGCTTTTCCG |
| | | GGTCCAAGTCCGGCAACACCGCCAG |
| | | CCTGACCATCAGCGGGCTCCAGGCA |
| | | GAAGATGAGGCTGACTATTACTGCT |
| | | CCTCCTACACGTCAAGCTCCACCCT |
| | | CTACGTGTTCGGGTCCGGGACCAAA |
| | | GTCACTGTGCTGACCACTACCCCAG |
| | | CACCGAGGCCACCCACCCCGGCTCC |
| | | TACCATCGCCTCCCAGCCTCTGTCC |
| | | CTGCGTCCGGAGGCATGTAGACCCG |
| | | CAGCTGGTGGGGCCGTGCATACCCG |
| | | GGGTCTTGACTTCGCCTGCGATATC |
| | | TACATTTGGGCCCCTCTGGCTGGTA |
| | | CTTGCGGGGTCCTGCTGCTTTCACT |
| | | CGTGATCACTCTTTACTGTAAGCGC |
| | | GGTCGGAAGAAGCTGCTGTACATCT |
| | | TTAAGCAACCCTTCATGAGGCCTGT |
| | | GCAGACTACTCAAGAGGAGGACGGC |
| | | TGTTCATGCCGGTTCCCAGAGGAGG |
| | | AGGAAGGCGGCTGCGAACTGCGCGT |
| | | GAAATTCAGCCGCAGCGCAGATGCT |
| | | CCAGCCTACCAGCAGGGGCAGAACC |
| | | AGCTCTACAACGAACTCAATCTTGG |
| | | TCGGAGAGAGGAGTACGACGTGCTG |
| | | GACAAGCGGAGAGGACGGGACCCAG |
| | | AAATGGGCGGGAAGCCGCGCAGAAA |
| | | GAATCCCCAAGAGGGCCTGTACAAC |
| | | GAGCTCCAAAAGGATAAGATGGCAG |
| | | AAGCCTATAGCGAGATTGGTATGAA |
| | | AGGGGAACGCAGAAGAGGCAAAGGC |
| | | CACGACGGACTGTACCAGGGACTCA |
| | | GCACCGCCACCAAGGACACCTATGA |
| | | CGCTCTTCACATGCAGGCCCTGCCG |
| | | CCTCGG |

TABLE 8
Kabat CDRs of exemplary B cell-derived anti-BCMA molecules

| Kabat | HCDR1 | HCDR2 | HCDR3 | LCDR1 | LCDR2 | LCDR3 |
|---|---|---|---|---|---|---|
| PI61 | SYGMH | VISYDGSN | SGYALHDD | TGTSSDV | DVSNRPS | SSYTSSS |
| | (SEQ ID | KYYADSV | YYGLDV | GGYNYV | (SEQ ID NO: | TLYV |
| | NO: 86) | KG (SEQ ID | (SEQ ID NO: | S (SEQ ID | 96) | (SEQ ID |
| | | NO: 87) | 88) | NO: 95) | | NO: 97) |
| B61-02 | SYGMH | VISYKGSN | SGYALHDD | TGTSSDV | EVSNRLR | SSYTSSS |
| | (SEQ ID | KYYADSV | YYGLDV | GGYNYV | (SEQ ID NO: | ALYV |
| | NO: 86) | KG (SEQ ID | (SEQ ID NO: | S (SEQ ID | 114) | (SEQ ID |
| | | NO: 109) | 88) | NO: 95) | | NO: 115) |
| B61-10 | SYGMH | VISYKGSN | SGYALHDD | TGTSSDV | EVSNRLR | SSYTSSS |
| | (SEQ ID | KYYADSV | YYGLDV | GGYNYV | (SEQ ID NO: | TLYV |
| | NO: 86) | KG (SEQ ID | (SEQ ID NO: | S (SEQ ID | 114) | (SEQ ID |
| | | NO: 109) | 88) | NO: 95) | | NO: 97) |
| Consensus | SYGMH | VISYXGSN | SGYALHDD | TGTSSDV | X₁VSNRX₂X₃, | SSYTSSS |
| | (SEQ ID | KYYADSV | YYGLDV | GGYNYV | wherein X₁ is | XLYV, |
| | NO: 86) | KG, wherein | (SEQ ID NO: | S (SEQ ID | D or E; X₂ is P | wherein X |
| | | X is D or K | 88) | NO: 95) | or L; and X₃ is | is T or A |
| | | (SEQ ID NO: | | | S or R (SEQ | (SEQ ID |
| | | 130) | | | ID NO: 131) | NO: 132) |

TABLE 9
Chothia CDRs of exemplary B cell-derived anti-BCMA molecules

| Chothia | HCDR1 | HCDR2 | HCDR3 | LCDR1 | LCDR2 | LCDR3 |
|---|---|---|---|---|---|---|
| PI61 | GFTFSSY | SYDGSN | SGYALHDDY | TSSDVGG | DVS (SEQ | YTSSSTLY |
| | (SEQ ID | (SEQ ID NO: | YGLDV (SEQ | YNY (SEQ | ID NO: 99) | (SEQ ID |
| | NO: 47) | 89) | ID NO: 88) | ID NO: 98) | | NO: 100) |
| B61-02 | GFTFSSY | SYKGSN | SGYALHDDY | TSSDVGG | EVS (SEQ | YTSSSAL |
| | (SEQ ID | (SEQ ID NO: | YGLDV (SEQ | YNY (SEQ | ID NO: | Y (SEQ ID |
| | NO: 47) | 110) | ID NO: 88) | ID NO: 98) | 116) | NO: 117) |
| B61-10 | GFTFSSY | SYKGSN | SGYALHDDY | TSSDVGG | EVS (SEQ | YTSSSTLY |
| | (SEQ ID | (SEQ ID NO: | YGLDV (SEQ | YNY (SEQ | ID NO: | (SEQ ID |
| | NO: 47) | 110) | ID NO: 88) | ID NO: 98) | 116) | NO: 100) |
| Consensus | GFTFSSY | SYXGSN, | SGYALHDDY | TSSDVGG | XVS, | YTSSSXL |
| | (SEQ ID | wherein X is | YGLDV (SEQ | YNY (SEQ | wherein X | Y, wherein |
| | NO: 47) | D or K (SEQ | ID NO: 88) | ID NO: 98) | is D or E | X is T or A |
| | | ID NO: 133) | | | (SEQ ID | (SEQ ID |
| | | | | | NO: 134) | NO: 135) |

TABLE 10
IMGT CDRs of exemplary B cell-derived anti-BCMA molecules

| IMGT | HCDR1 | HCDR2 | HCDR3 | LCDR1 | LCDR2 | LCDR3 |
|---|---|---|---|---|---|---|
| PI61 | GFTFSSYG | ISYDGSN | GGSGYALHDD | SSDVGGY | DVS | SSYTSSSTL |
| | (SEQ ID | K (SEQ ID | YYGLDV (SEQ | NY (SEQ | (SEQ ID | YV (SEQ ID |
| | NO: 90) | NO: 91) | ID NO: 92) | ID NO: 101) | NO: 99) | NO: 97) |
| B61-02 | GFTFSSYG | ISYKGSN | GGSGYALHDD | SSDVGGY | EVS | SSYTSSSA |
| | (SEQ ID | K (SEQ ID | YYGLDV (SEQ | NY (SEQ | (SEQ ID | LYV (SEQ |
| | NO: 90) | NO: 111) | ID NO: 92) | ID NO: 101) | NO: 116) | ID NO: 115) |
| B61-10 | GFTFSSYG | ISYKGSN | GGSGYALHDD | SSDVGGY | EVS | SSYTSSSTL |
| | (SEQ ID | K (SEQ ID | YYGLDV (SEQ | NY (SEQ | (SEQ ID | YV (SEQ ID |
| | NO: 90) | NO: 111) | ID NO: 92) | ID NO: 101) | NO: 116) | NO: 97) |
| Consensus | GFTFSSYG | ISYXGSN | GGSGYALHDD | SSDVGGY | XVS, | SSYTSSSX |
| | (SEQ ID | K, wherein | YYGLDV (SEQ | NY (SEQ | wherein | LYV, |
| | NO: 90) | X is D or K | ID NO: 92) | ID NO: 101) | X is D or | wherein X is |
| | | (SEQ ID | | | E (SEQ | T or A (SEQ |
| | | NO: 136) | | | ID NO: | ID NO: 132) |
| | | | | | 134) | |

TABLE 11
Amino acid and nucleic acid sequences of exemplary anti-BCMA molecules based
on PI61

| Identification | Protein sequence | DNA sequence (5′-3′) |
|---|---|---|
| Signal peptide | MALPVTALLLPLALLLHAA | Atggccctccctgtcaccgctctgttgctgccgcttgctctgctg |
| | RP (SEQ ID NO: 1) | ctccacgcagegcgaccg (SEQ ID NO: 252) |
| PI61 VH | QVQLQESGGGVVQPGRSLRLS | CAGGTACAATTGCAGGAGTCTGGAGGCGG |
| | CAASGFTFSSYGMHWVRQAP | TGTGGTGCAACCCGGTCGCAGCTTGCGCCT |
| | GKGLEWVAVISYDGSNKYYA | GAGTTGTGCTGCGTCTGGATTTACATTTTC |
| | DSVKGRFTISRDNSKNTLYLQ | ATCTTACGGAATGCATTGGGTACGCCAGG |
| | MNSLRAEDTAVYYCGGSGYA | CACCGGGGAAAGGCCTTGAATGGGTGGCT |
| | LHDDYYGLDVWGQGTLVTVS | GTAATTTCATACGATGGTTCCAACAAATAC |
| | S (SEQ ID NO: 93) | TATGCTGACTCAGTCAAGGGTCGATTTACA |
| | | ATTAGTCGGGACAACTCCAAGAACACCCT |
| | | TTATCTTCAAATGAATTCCCTTAGAGCAGA |
| | | GGATACGGCGGTCTATTACTGTGGTGGCA |
| | | GTGGTTATGCACTTCATGATGATTACTATG |
| | | GCTTGGATGTCTGGGGGCAAGGGACGCTT |
| | | GTAACTGTATCCTCT (SEQ ID NO: 260) |
| PI61 VL | QSALTQPASVSGSPGQSITISCT | CAATCTGCTCTGACTCAACCAGCAAGCGT |
| | GTSSDVGGYNYVSWYQQHPG | ATCAGGGTCACCGGGACAGAGTATTACCA |
| | KAPKLMIYDVSNRPSGVSNRFS | TAAGTTGCACGGGGACCTCTAGCGATGTA |
| | GSKSGNTASLTISGLQAEDEAD | GGGGGGTATAATTATGTATCTTGGTATCAA |
| | YYCSSYTSSSTLYVFGSGTKVT | CAACACCCCGGGAAAGCCCCTAAATTGAT |
| | VL (SEQ ID NO: 102) | GATCTACGACGTGAGCAATCGACCTAGTG |
| | | GCGTATCAAATCGCTTCTCTGGTAGCAAGA |
| | | GTGGGAATACGGCGTCCCTTACTATTAGCG |
| | | GATTGCAAGCAGAAGATGAGGCCGATTAC |
| | | TACTGCAGCTCCTATACTAGCTCTTCTACA |
| | | TTGTACGTCTTTGGGAGCGGAACAAAAGT |
| | | AACAGTACTC (SEQ ID NO: 261) |
| Linker | GGGGSGGGGSGGGGS (SEQ ID | |
| | NO: 104) | |
| ScFv PI61 | QVQLQESGGGVVQPGRSLR | CaggtacaattgcaggagtctggaggcggtgtgGtgcaacc |
| | LSCAASGFTFSSYGMHWVR | cggtcgcagcttgcgcctgagttgtGctgcgtctggatttacatt |
| | QAPGKGLEWVAVISYDGSN | ttcatcttacggaAtgcattgggtacgccaggcaccggggaa |
| | KYYADSVKGRFTISRDNSK | aggcCttgaatgggtggctgtaatttcatacgatggtTccaac |
| | NTLYLQMNSLRAEDTAVYY | aaatactatgctgactcagtcaagggtCgatttacaattagtcg |
| | CGGSGYALHDDYYGLDVW | ggacaactccaagaacAccctttatcttcaaatgaattcccttag |
| | GQGTLVTVSSGGGGSGGGG | agcaGaggatacggcggtctattactgtggtggcagtGgttat |
| | SGGGGSQSALTQPASVSGSP | gcacttcatgatgattactatggcttgGatgtctgggggcaagg |
| | GQSITISCTGTSSDVGGYNY | gacgcttgtaactgtaTcctctggtggtggtggtagtggtggg |
| | VSWYQQHPGKAPKLMIYD | ggaggcTccggcggtggcggctctcaatctgctctgactCaa |
| | VSNRPSGVSNRFSGSKSGNT | ccagcaagcgtatcagggtcaccgggacagAgtattaccata |
| | ASLTISGLQAEDEADYYCSS | agttgcacggggacctctagcGatgtaggggggtataattatg |
| | YTSSSTLYVFGSGTKVTVL | tatcttggtatCaacaacaccccgggaaagcccctaaattgatg |
| | (SEQ ID NO: 105) | AtctacgacgtgagcaatcgacctagtggcgtaTcaaatcgc |
| | | ttctctggtagcaagagtgggaatAcggcgtcccttactattag |
| | | cggattgcaagcaGaagatgaggccgattactactgcagctc |
| | | ctatActagctcttctacattgtacgtctttgggagcggaacaaa |
| | | agtaacagtactc (SEQ ID NO: 253) |
| Transmembrane | TTTPAPRPPTPAPTIASQPLS | AcaacaacacctgccccgagaccgcctacaccaGccccga |
| domain and hinge | LRPEACRPAAGGAVHTRGL | ctattgccagccagcctctgagcctcAggcctgaggcctgtag |
| | DFACDIYIWAPLAGTCGVLL | gcccgcagcgggcggcGcagttcatacacggggcttggattt |
| | LSLVITLYC (SEQ ID NO: | cgcttgtGatatttatatttgggctcctttggcggggacaTgtgg |
| | 202) | cgtgctgcttctgtcacttgttattacactgtactgt (SEQ ID |
| | | NO: 254) |
| 4-1BB | KRGRKKLLYIFKQPFMRPV | AaacgcgggcgaaaaaaattgctgtatatttttAagcagccat |
| | QTTQEEDGCSCRFPEEEEGG | ttatgaggcccgttcagacgacgCaggaggaggacggttgct |
| | CEL (SEQ ID NO: 7) | cttgcaggttcccagaagaggaagaagggggctgtgaattg |
| | | (SEQ ID NO: 255) |
| CD3zeta | RVKFSRSADAPAYQQGQNQ | CgggttaaattttcaagatccgcagacgctccaGcataccaac |
| | LYNELNLGRREEYDVLDKR | agggacaaaaccaactctataacGagctgaatcttggaagaa |
| | RGRDPEMGGKPRRKNPQEG | gggaggaatatgatGtgctggataaacggcgcggtagagatc |
| | LYNELQKDKMAEAYSEIGM | cggagAtgggcggaaaaccaaggcgaaaaaaccctcagG |
| | KGERRRGKGHDGLYQGLST | agggactctacaacgaactgcagaaagacaaaAtggcggag |
| | ATKDTYDALHMQALPPR | gcttattccgaaataggcatgaagGgcgagcggaggcgagg |
| | (SEQ ID NO: 10) | gaaagggcacgacggaCtgtatcaaggcctctcaaccgcga |
| | | ctaaggatAcgtacgacgccctgcacatgcaggccctgcctc |
| | | cgaga (SEQ ID NO: 256) |
| PI61 full CAR | MALPVTALLLPLALLLHAA | ATGGCCCTCCCTGTCACCGCTCTGTTG |
| construct | RPQVQLQESGGGVVQPGRS | CTGCCGCTTGCTCTGCTGCTCCACGCA |
| | LRLSCAASGFTFSSYGMHW | GCGCGACCGCAGGTACAATTGCAGGA |
| | VRQAPGKGLEWVAVISYDG | GTCTGGAGGCGGTGTGGTGCAACCCG |
| | SNKYYADSVKGRFTISRDNS | GTCGCAGCTTGCGCCTGAGTTGTGCTG |
| | KNTLYLQMNSLRAEDTAVY | CGTCTGGATTTACATTTTCATCTTACGG |
| | YCGGSGYALHDDYYGLDV | AATGCATTGGGTACGCCAGGCACCGG |
| | WGQGTLVTVSSGGGGSGG | GGAAAGGCCTTGAATGGGTGGCTGTA |
| | GGSGGGGSQSALTQPASVS | ATTTCATACGATGGTTCCAACAAATAC |
| | GSPGQSITISCTGTSSDVGGY | TATGCTGACTCAGTCAAGGGTCGATTT |
| | NYVSWYQQHPGKAPKLMI | ACAATTAGTCGGGACAACTCCAAGAA |
| | YDVSNRPSGVSNRFSGSKSG | CACCCTTTATCTTCAAATGAATTCCCTT |
| | NTASLTISGLQAEDEADYYC | AGAGCAGAGGATACGGCGGTCTATTA |
| | SSYTSSSTLYVFGSGTKVTV | CTGTGGTGGCAGTGGTTATGCACTTCA |
| | LTTTPAPRPPTPAPTIASQPL | TGATGATTACTATGGCTTGGATGTCTG |
| | SLRPEACRPAAGGAVHTRG | GGGGCAAGGGACGCTTGTAACTGTATC |
| | LDFACDIYIWAPLAGTCGVL | CTCTGGTGGTGGTGGTAGTGGTGGGGG |
| | LLSLVITLYCKRGRKKLLYI | AGGCTCCGGCGGTGGCGGCTCTCAATC |
| | FKQPFMRPVQTTQEEDGCS | TGCTCTGACTCAACCAGCAAGCGTATC |
| | CRFPEEEEGGCELRVKFSRS | AGGGTCACCGGGACAGAGTATTACCA |
| | ADAPAYQQGQNQLYNELN | TAAGTTGCACGGGGACCTCTAGCGATG |
| | LGRREEYDVLDKRRGRDPE | TAGGGGGGTATAATTATGTATCTTGGT |
| | MGGKPRRKNPQEGLYNELQ | ATCAACAACACCCCGGGAAAGCCCCT |
| | KDKMAEAYSEIGMKGERRR | AAATTGATGATCTACGACGTGAGCAAT |
| | GKGHDGLYQGLSTATKDTY | CGACCTAGTGGCGTATCAAATCGCTTC |
| | DALHMQALPPR (SEQ ID | TCTGGTAGCAAGAGTGGGAATACGGC |
| | NO: 257) | GTCCCTTACTATTAGCGGATTGCAAGC |
| | | AGAAGATGAGGCCGATTACTACTGCA |
| | | GCTCCTATACTAGCTCTTCTACATTGTA |
| | | CGTCTTTGGGAGCGGAACAAAAGTAA |
| | | CAGTACTCACAACAACACCTGCCCCGA |
| | | GACCGCCTACACCAGCCCCGACTATTG |
| | | CCAGCCAGCCTCTGAGCCTCAGGCCTG |
| | | AGGCCTGTAGGCCCGCAGCGGGCGGC |
| | | GCAGTTCATACACGGGGCTTGGATTTC |
| | | GCTTGTGATATTTATATTTGGGCTCCTT |
| | | TGGCGGGGACATGTGGCGTGCTGCTTC |
| | | TGTCACTTGTTATTACACTGTACTGTA |
| | | AACGCGGGCGAAAAAAATTGCTGTAT |
| | | ATTTTTAAGCAGCCATTTATGAGGCCC |
| | | GTTCAGACGACGCAGGAGGAGGACGG |
| | | TTGCTCTTGCAGGTTCCCAGAAGAGGA |
| | | AGAAGGGGGCTGTGAATTGCGGGTTA |
| | | AATTTTCAAGATCCGCAGACGCTCCAG |
| | | CATACCAACAGGGACAAAACCAACTC |
| | | TATAACGAGCTGAATCTTGGAAGAAG |
| | | GGAGGAATATGATGTGCTGGATAAAC |
| | | GGCGCGGTAGAGATCCGGAGATGGGC |
| | | GGAAAACCAAGGCGAAAAAACCCTCA |
| | | GGAGGGACTCTACAACGAACTGCAGA |
| | | AAGACAAAATGGCGGAGGCTTATTCC |
| | | GAAATAGGCATGAAGGGCGAGCGGAG |
| | | GCGAGGGAAAGGGCACGACGGACTGT |
| | | ATCAAGGCCTCTCAACCGCGACTAAGG |
| | | ATACGTACGACGCCCTGCACATGCAGG |
| | | CCCTGCCTCCGAGA (SEQ ID NO: 258) |
| PI61 full CAR | | ATGGCCCTCCCTGTCACCGCTCTGTTGCTGCCGC |
| construct | | TTGCTCTGCTGCTCCACGCAGCGCGACCGCAGGT |
| (Nucleic acid | | ACAATTGCAGGAGTCTGGAGGCGGTGTGGTGCAA |
| with signal | | CCCGGTCGCAGCTTGCGCCTGAGTTGTGCTGCGT |
| peptide and stop | | CTGGATTTACATTTTCATCTTACGGAATGCATTG |
| codons) | | GGTACGCCAGGCACCGGGGAAAGGCCTTGAATGG |
| | | GTGGCTGTAATTTCATACGATGGTTCCAACAAAT |
| | | ACTATGCTGACTCAGTCAAGGGTCGATTTACAAT |
| | | TAGTCGGGACAACTCCAAGAACACCCTTTATCTT |
| | | CAAATGAATTCCCTTAGAGCAGAGGATACGGCGG |
| | | TCTATTACTGTGGTGGCAGTGGTTATGCACTTCA |
| | | TGATGATTACTATGGCTTGGATGTCTGGGGGCAA |
| | | GGGACGCTTGTAACTGTATCCTCTGGTGGTGGTG |
| | | GTAGTGGTGGGGGAGGCTCCGGCGGTGGCGGCTC |
| | | TCAATCTGCTCTGACTCAACCAGCAAGCGTATCA |
| | | GGGTCACCGGGACAGAGTATTACCATAAGTTGCA |
| | | CGGGGACCTCTAGCGATGTAGGGGGGTATAATTA |
| | | TGTATCTTGGTATCAACAACACCCCGGGAAAGCC |
| | | CCTAAATTGATGATCTACGACGTGAGCAATCGAC |
| | | CTAGTGGCGTATCAAATCGCTTCTCTGGTAGCAA |
| | | GAGTGGGAATACGGCGTCCCTTACTATTAGCGGA |
| | | TTGCAAGCAGAAGATGAGGCCGATTACTACTGCA |
| | | GCTCCTATACTAGCTCTTCTACATTGTACGTCTT |
| | | TGGGAGCGGAACAAAAGTAACAGTACTCACAACA |
| | | ACACCTGCCCCGAGACCGCCTACACCAGCCCCGA |
| | | CTATTGCCAGCCAGCCTCTGAGCCTCAGGCCTGA |
| | | GGCCTGTAGGCCCGCAGCGGGCGGCGCAGTTCAT |
| | | ACACGGGGCTTGGATTTCGCTTGTGATATTTATA |
| | | TTTGGGCTCCTTTGGCGGGGACATGTGGCGTGCT |
| | | GCTTCTGTCACTTGTTATTACACTGTACTGTAAA |
| | | CGCGGGCGAAAAAAATTGCTGTATATTTTTAAGC |
| | | AGCCATTTATGAGGCCCGTTCAGACGACGCAGGA |
| | | GGAGGACGGTTGCTCTTGCAGGTTCCCAGAAGAG |
| | | GAAGAAGGGGGCTGTGAATTGCGGGTTAAATTTT |
| | | CAAGATCCGCAGACGCTCCAGCATACCAACAGGG |
| | | ACAAAACCAACTCTATAACGAGCTGAATCTTGGA |
| | | AGAAGGGAGGAATATGATGTGCTGGATAAACGGC |
| | | GCGGTAGAGATCCGGAGATGGGCGGAAAACCAAG |
| | | GCGAAAAAACCCTCAGGAGGGACTCTACAACGAA |
| | | CTGCAGAAAGACAAAATGGCGGAGGCTTATTCCG |
| | | AAATAGGCATGAAGGGCGAGCGGAGGCGAGGGAA |
| | | AGGGCACGACGGACTGTATCAAGGCCTCTCAACC |
| | | GCGACTAAGGATACGTACGACGCCCTGCACATGC |
| | | AGGCCCTGCCTCCGAGATGATAA (SEQ ID |
| | | NO: 416) |
| PI61 mature | QVQLQESGGGVVQPGRSLR | caggtacaattgcaggagtctggaggcggtgtggtgcaacccggtc |
| CAR protein | LSCAASGFTFSSYGMHWVR | gcagcttgcgcctgagttgtgctgcgtctggatttacattttcatcttacg |
| | QAPGKGLEWVAVISYDGSN | gaatgcattgggtacgccaggcaccggggaaaggccttgaatgggt |
| | KYYADSVKGRFTISRDNSK | ggctgtaatttcatacgatggttccaacaaatactatgctgactcagtca |
| | NTLYLQMNSLRAEDTAVYY | agggtcgatttacaattagtcgggacaactccaagaacaccctttatctt |
| | CGGSGYALHDDYYGLDVW | caaatgaattcccttagagcagaggatacggcggtctattactgtggtg |
| | GQGTLVTVSSGGGGSGGGG | gcagtggttatgcacttcatgatgattactatggcttggatgtctggggg |
| | SGGGGSQSALTQPASVSGSP | caagggacgcttgtaactgtatcctctggtggtggtggtagtggtggg |
| | GQSITISCTGTSSDVGGYNY | ggaggctccggcggtggcggctctcaatctgctctgactcaaccagc |
| | VSWYQQHPGKAPKLMIYD | aagcgtatcagggtcaccgggacagagtattaccataagttgcacgg |
| | VSNRPSGVSNRFSGSKSGNT | ggacctctagcgatgtaggggggtataattatgtatcttggtatcaaca |
| | ASLTISGLQAEDEADYYCSS | acaccccgggaaagcccctaaattgatgatctacgacgtgagcaatc |
| | YTSSSTLYVFGSGTKVTVLT | gacctagtggcgtatcaaatcgcttctctggtagcaagagtgggaata |
| | TTPAPRPPTPAPTIASQPLSL | cggcgtcccttactattagcggattgcaagcagaagatgaggccgatt |
| | RPEACRPAAGGAVHTRGLD | actactgcagctcctatactagctcttctacattgtacgtctttgggagcg |
| | FACDIYIWAPLAGTCGVLLL | gaacaaaagtaacagtactcacaacaacacctgccccgagaccgcct |
| | SLVITLYCKRGRKKLLYIFK | acaccagccccgactattgccagccagcctctgagcctcaggcctga |
| | QPFMRPVQTTQEEDGCSCR | ggcctgtaggcccgcagcgggcggcgcagttcatacacggggcttg |
| | FPEEEEGGCELRVKFSRSAD | gatttcgcttgtgatatttatatttgggctcctttggcggggacatgtggc |
| | APAYQQGQNQLYNELNLG | gtgctgcttctgtcacttgttattacactgtactgtaaacgcgggcgaaa |
| | RREEYDVLDKRRGRDPEMG | aaaattgctgtatatttttaagcagccatttatgaggcccgttcagacga |
| | GKPRRKNPQEGLYNELQKD | cgcaggaggaggacggttgctcttgcaggttcccagaagaggaaga |
| | KMAEAYSEIGMKGERRRGK | agggggctgtgaattgcgggttaaattttcaagatccgcagacgctcc |
| | GHDGLYQGLSTATKDTYDA | agcataccaacagggacaaaaccaactctataacgagctgaatcttg |
| | LHMQALPPR (SEQ ID NO: | gaagaagggaggaatatgatgtgctggataaacggcgcggtagaga |
| | 107) | tccggagatgggcggaaaaccaaggcgaaaaaaccctcaggaggg |
| | | actctacaacgaactgcagaaagacaaaatggcggaggcttattccg |
| | | aaataggcatgaagggcgagcggaggcgagggaaagggcacgac |
| | | ggactgtatcaaggcctctcaaccgcgactaaggatacgtacgacgc |
| | | cctgcacatgcaggccctgcctccgaga (SEQ ID NO: 259) |

TABLE 12
Amino acid and nucleic acid sequences of exemplary hybridoma-derived anti-
BCMA molecules

| | | |
|---|---|---|
| SEQ ID | Name/ | |
| NO | Description | Sequence |

Hy03

| | | |
|---|---|---|
| SEQ ID | HCDR1 | GFWMS |
| NO: 137 | (Kabat) | |
| SEQ ID | HCDR2 | NIKQDGSEKYYVDSVRG |
| NO: 138 | (Kabat) | |
| SEQ ID | HCDR3 | ALDYYGMDV |
| NO: 139 | (Kabat) | |
| SEQ ID | HCDR1 | GFTFSGF |
| NO: 140 | (Chothia) | |
| SEQ ID | HCDR2 | KQDGSE |
| NO: 141 | (Chothia) | |
| SEQ ID | HCDR3 | ALDYYGMDV |
| NO: 139 | (Chothia) | |
| SEQ ID | HCDR1 | GFTFSGFW |
| NO: 142 | (IMGT) | |
| SEQ ID | HCDR2 | IKQDGSEK |
| NO: 143 | (IMGT) | |
| SEQ ID | HCDR3 | ARALDYYGMDV |
| NO: 144 | (IMGT) | |
| SEQ ID | VH | EVQLVESGGGLVQPGGSLRLSCAASGFTFSGFWMSWVRQAPGKG |
| NO: 145 | | LEWVANIKQDGSEKYYVDSVRGRFTISRDNAKNSLYLQMNSLRAE |
| | | DTAVYYCARALDYYGMDVWGQGTTVTVSS |
| SEQ ID | DNA VH | GAAGTGCAACTGGTGGAGAGCGGTGGAGGGCTTGTCCAGCCCG |
| NO: 146 | | GAGGATCGCTGCGGCTGTCCTGTGCTGCGTCCGGGTTCACCTTC |
| | | TCCGGCTTCTGGATGTCCTGGGTCAGACAGGCACCGGGAAAGG |
| | | GCCTCGAATGGGTGGCCAACATCAAGCAGGATGGCTCCGAGAA |
| | | GTACTACGTCGACTCCGTGAGAGGCCGCTTCACCATCTCCCGGG |
| | | ACAACGCCAAGAACTCGCTGTACCTCCAAATGAATAGCCTCAG |
| | | GGCGGAAGATACTGCTGTGTATTACTGCGCACGCGCCCTTGACT |
| | | ACTACGGCATGGACGTCTGGGGCCAAGGGACCACTGTGACCGT |
| | | GTCTAGC |
| SEQ ID | LCDR1 | RSSQSLLDSDDGNTYLD |
| NO: 147 | (Kabat) | |
| SEQ ID | LCDR2 | TLSYRAS |
| NO: 148 | (Kabat) | |
| SEQ ID | LCDR3 | TQRLEFPSIT |
| NO: 149 | (Kabat) | |
| SEQ ID | LCDR1 | SQSLLDSDDGNTY |
| NO: 150 | (Chothia) | |
| SEQ ID | LCDR2 | TLS |
| NO: 151 | (Chothia) | |
| SEQ ID | LCDR3 | RLEFPSI |
| NO: 152 | (Chothia) | |
| SEQ ID | LCDR1 | QSLLDSDDGNTY |
| NO: 153 | (IMGT) | |
| SEQ ID | LCDR2 | TLS |
| NO: 151 | (IMGT) | |
| SEQ ID | LCDR3 | TQRLEFPSIT |
| NO: 149 | (IMGT) | |
| SEQ ID | VL | DIVMTQTPLSLPVTPGEPASISCRSSQSLLDSDDGNTYLDWYLQKP |
| NO: 154 | | GQSPRLLIYTLSYRASGVPDRFSGSGSGTDFTLKISRVEAEDVGLYY |
| | | CTQRLEFPSITFGQGTRLEIK |
| SEQ ID | DNA VL | GATATCGTGATGACCCAGACTCCCCTGTCCCTGCCTGTGACTCC |
| NO: 155 | | CGGAGAACCAGCCTCCATTTCCTGCCGGTCCTCCCAGTCCCTGC |
| | | TGGACAGCGACGACGGCAACACTTACCTGGACTGGTACTTGCA |
| | | GAAGCCGGGCCAATCGCCTCGCCTGCTGATCTATACCCTGTCAT |
| | | ACCGGGCCTCAGGAGTGCCTGACCGCTTCTCGGGATCAGGGAG |
| | | CGGGACCGATTTCACCCTGAAAATTTCCCGAGTGGAAGCCGAG |
| | | GACGTCGGACTGTACTACTGCACCCAGCGCCTCGAATTCCCGTC |
| | | GATTACGTTTGGACAGGGTACCCGGCTTGAGATCAAG |
| SEQ ID | Linker | GGGGSGGGGSGGGGSGGGGS |
| NO: 63 | | |
| SEQ ID | scFv (VH- | EVQLVESGGGLVQPGGSLRLSCAASGFTFSGFWMSWVRQAPGKG |
| NO: 156 | linker-VL) | LEWVANIKQDGSEKYYVDSVRGRFTISRDNAKNSLYLQMNSLRAE |
| | | DTAVYYCARALDYYGMDVWGQGTTVTVSSGGGGSGGGGSGGG |
| | | GSGGGGSDIVMTQTPLSLPVTPGEPASISCRSSQSLLDSDDGNTYLD |
| | | WYLQKPGQSPRLLIYTLSYRASGVPDRFSGSGSGTDFTLKISRVEA |
| | | EDVGLYYCTQRLEFPSITFGQGTRLEIK |
| SEQ ID | DNA scFv | GAAGTGCAACTGGTGGAGAGCGGTGGAGGGCTTGTCCAGCCCG |
| NO: 157 | | GAGGATCGCTGCGGCTGTCCTGTGCTGCGTCCGGGTTCACCTTC |
| | | TCCGGCTTCTGGATGTCCTGGGTCAGACAGGCACCGGGAAAGG |
| | | GCCTCGAATGGGTGGCCAACATCAAGCAGGATGGCTCCGAGAA |
| | | GTACTACGTCGACTCCGTGAGAGGCCGCTTCACCATCTCCCGGG |
| | | ACAACGCCAAGAACTCGCTGTACCTCCAAATGAATAGCCTCAG |
| | | GGCGGAAGATACTGCTGTGTATTACTGCGCACGCGCCCTTGACT |
| | | ACTACGGCATGGACGTCTGGGGCCAAGGGACCACTGTGACCGT |
| | | GTCTAGCGGAGGCGGAGGTTCAGGGGGCGGTGGATCAGGCGGA |
| | | GGAGGATCGGGGGGTGGTGGATCGGATATCGTGATGACCCAGA |
| | | CTCCCCTGTCCCTGCCTGTGACTCCCGGAGAACCAGCCTCCATT |
| | | TCCTGCCGGTCCTCCCAGTCCCTGCTGGACAGCGACGACGGCAA |
| | | CACTTACCTGGACTGGTACTTGCAGAAGCCGGGCCAATCGCCTC |
| | | GCCTGCTGATCTATACCCTGTCATACCGGGCCTCAGGAGTGCCT |
| | | GACCGCTTCTCGGGATCAGGGAGCGGGACCGATTTCACCCTGA |
| | | AAATTTCCCGAGTGGAAGCCGAGGACGTCGGACTGTACTACTG |
| | | CACCCAGCGCCTCGAATTCCCGTCGATTACGTTTGGACAGGGTA |
| | | CCCGGCTTGAGATCAAG |
| SEQ ID | Full CAR | EVQLVESGGGLVQPGGSLRLSCAASGFTFSGFWMSWVRQAPGKG |
| NO: 158 | amino acid | LEWVANIKQDGSEKYYVDSVRGRFTISRDNAKNSLYLQMNSLRAE |
| | sequence | DTAVYYCARALDYYGMDVWGQGTTVTVSSGGGGSGGGGSGGG |
| | | GSGGGGSDIVMTQTPLSLPVTPGEPASISCRSSQSLLDSDDGNTYLD |
| | | WYLQKPGQSPRLLIYTLSYRASGVPDRFSGSGSGTDFTLKISRVEA |
| | | EDVGLYYCTQRLEFPSITFGQGTRLEIKTTTPAPRPPTPAPTIASQPL |
| | | SLRPEACRPAAGGAVHTRGLDFACDIYIWAPLAGTCGVLLLSLVIT |
| | | LYCKRGRKKLLYIFKQPFMRPVQTTQEEDGCSCRFPEEEEGGCELR |
| | | VKFSRSADAPAYQQGQNQLYNELNLGRREEYDVLDKRRGRDPEM |
| | | GGKPRRKNPQEGLYNELQKDKMAEAYSEIGMKGERRRGKGHDG |
| | | LYQGLSTATKDTYDALHMQALPPR |
| SEQ ID | Full CAR | GAAGTGCAACTGGTGGAGAGCGGTGGAGGGCTTGTCCAGCCCG |
| NO: 159 | DNA | GAGGATCGCTGCGGCTGTCCTGTGCTGCGTCCGGGTTCACCTTC |
| | sequence | TCCGGCTTCTGGATGTCCTGGGTCAGACAGGCACCGGGAAAGG |
| | | GCCTCGAATGGGTGGCCAACATCAAGCAGGATGGCTCCGAGAA |
| | | GTACTACGTCGACTCCGTGAGAGGCCGCTTCACCATCTCCCGGG |
| | | ACAACGCCAAGAACTCGCTGTACCTCCAAATGAATAGCCTCAG |
| | | GGCGGAAGATACTGCTGTGTATTACTGCGCACGCGCCCTTGACT |
| | | ACTACGGCATGGACGTCTGGGGCCAAGGGACCACTGTGACCGT |
| | | GTCTAGCGGAGGCGGAGGTTCAGGGGGCGGTGGATCAGGCGGA |
| | | GGAGGATCGGGGGGTGGTGGATCGGATATCGTGATGACCCAGA |
| | | CTCCCCTGTCCCTGCCTGTGACTCCCGGAGAACCAGCCTCCATT |
| | | TCCTGCCGGTCCTCCCAGTCCCTGCTGGACAGCGACGACGGCAA |
| | | CACTTACCTGGACTGGTACTTGCAGAAGCCGGGCCAATCGCCTC |
| | | GCCTGCTGATCTATACCCTGTCATACCGGGCCTCAGGAGTGCCT |
| | | GACCGCTTCTCGGGATCAGGGAGCGGGACCGATTTCACCCTGA |
| | | AAATTTCCCGAGTGGAAGCCGAGGACGTCGGACTGTACTACTG |
| | | CACCCAGCGCCTCGAATTCCCGTCGATTACGTTTGGACAGGGTA |
| | | CCCGGCTTGAGATCAAGACCACTACCCCAGCACCGAGGCCACC |
| | | CACCCCGGCTCCTACCATCGCCTCCCAGCCTCTGTCCCTGCGTC |
| | | CGGAGGCATGTAGACCCGCAGCTGGTGGGGCCGTGCATACCCG |
| | | GGGTCTTGACTTCGCCTGCGATATCTACATTTGGGCCCCTCTGG |
| | | CTGGTACTTGCGGGGTCCTGCTGCTTTCACTCGTGATCACTCTTT |
| | | ACTGTAAGCGCGGTCGGAAGAAGCTGCTGTACATCTTTAAGCA |
| | | ACCCTTCATGAGGCCTGTGCAGACTACTCAAGAGGAGGACGGC |
| | | TGTTCATGCCGGTTCCCAGAGGAGGAGGAAGGCGGCTGCGAAC |
| | | TGCGCGTGAAATTCAGCCGCAGCGCAGATGCTCCAGCCTACCA |
| | | GCAGGGGCAGAACCAGCTCTACAACGAACTCAATCTTGGTCGG |
| | | AGAGAGGAGTACGACGTGCTGGACAAGCGGAGAGGACGGGAC |
| | | CCAGAAATGGGCGGGAAGCCGCGCAGAAAGAATCCCCAAGAG |
| | | GGCCTGTACAACGAGCTCCAAAAGGATAAGATGGCAGAAGCCT |
| | | ATAGCGAGATTGGTATGAAAGGGGAACGCAGAAGAGGCAAAG |
| | | GCCACGACGGACTGTACCAGGGACTCAGCACCGCCACCAAGGA |
| | | CACCTATGACGCTCTTCACATGCAGGCCCTGCCGCCTCGG |

Hy52

| | | |
|---|---|---|
| SEQ ID | HCDR1 | SFRMN |
| NO: 160 | (Kabat) | |
| SEQ ID | HCDR2 | SISSSSSYIYYADSVKG |
| NO: 161 | (Kabat) | |
| SEQ ID | HCDR3 | WLSYYGMDV |
| NO: 162 | (Kabat) | |
| SEQ ID | HCDR1 | GFTFSSF |
| NO: 163 | (Chothia) | |
| SEQ ID | HCDR2 | SSSSSY |
| NO: 164 | (Chothia) | |
| SEQ ID | HCDR3 | WLSYYGMDV |
| NO: 162 | (Chothia) | |
| SEQ ID | HCDR1 | GFTFSSFR |
| NO: 165 | (IMGT) | |
| SEQ ID | HCDR2 | ISSSSSYI |
| NO: 166 | (IMGT) | |
| SEQ ID | HCDR3 | ARWLSYYGMDV |
| NO: 167 | (IMGT) | |
| SEQ ID | VH | EVQLVESGGGLVKPGGSLRLSCAASGFTFSSFRMNWVRQAPGKGL |
| NO: 168 | | EWVSSISSSSSYIYYADSVKGRFTISRDNAKNSLYLQMNSLRAEDT |
| | | AVYYCARWLSYYGMDVWGQGTTVTVSS |
| SEQ ID | DNA VH | GAAGTGCAACTGGTGGAGAGCGGTGGAGGGCTTGTCAAGCCCG |
| NO: 169 | | GAGGATCGCTGCGGCTGTCCTGTGCTGCGTCCGGGTTCACCTTC |
| | | TCCTCGTTCCGCATGAACTGGGTCAGACAGGCACCGGGAAAGG |
| | | GCCTCGAATGGGTGTCCTCAATCTCATCGTCCTCGTCCTACATC |
| | | TACTACGCCGACTCCGTGAAAGGCCGCTTCACCATCTCCCGGGA |
| | | CAACGCCAAGAACTCGCTGTACCTCCAAATGAATAGCCTCAGG |
| | | GCGGAAGATACTGCTGTGTATTACTGCGCACGCTGGCTTTCCTA |
| | | CTACGGCATGGACGTCTGGGGCCAAGGGACCACTGTGACCGTG |
| | | TCTAGC |
| SEQ ID | LCDR1 | RSSQSLLDSDDGNTYLD |
| NO: 147 | (Kabat) | |
| SEQ ID | LCDR2 | TLSFRAS |
| NO: 170 | (Kabat) | |
| SEQ ID | LCDR3 | MQRIGFPIT |
| NO: 171 | (Kabat) | |
| SEQ ID | LCDR1 | SQSLLDSDDGNTY |
| NO: 150 | (Chothia) | |
| SEQ ID | LCDR2 | TLS |
| NO: 151 | (Chothia) | |
| SEQ ID | LCDR3 | RIGFPI |
| NO: 172 | (Chothia) | |
| SEQ ID | LCDR1 | QSLLDSDDGNTY |
| NO: 153 | (IMGT) | |
| SEQ ID | LCDR2 | TLS |
| NO: 151 | (IMGT) | |
| SEQ ID | LCDR3 | MQRIGFPIT |
| NO: 171 | (IMGT) | |
| SEQ ID | VL | DIVMTQTPLSLPVTPGEPASISCRSSQSLLDSDDGNTYLDWYLQKP |
| NO: 173 | | GQSPQLLIYTLSFRASGVPDRFSGSGSGTDFTLKIRRVEAEDVGVY |
| | | YCMQRIGFPITFGQGTRLEIK |
| SEQ ID | DNA VL | GATATCGTGATGACCCAGACTCCCCTGTCCCTGCCTGTGACTCC |
| NO: 174 | | CGGAGAACCAGCCTCCATTTCCTGCCGGTCCTCCCAGTCCCTGC |
| | | TGGACAGCGACGACGGCAACACTTACCTGGACTGGTACTTGCA |
| | | GAAGCCGGGCCAATCGCCTCAGCTGCTGATCTATACCCTGTCAT |
| | | TCCGGGCCTCAGGAGTGCCTGACCGCTTCTCGGGATCAGGGAG |
| | | CGGGACCGATTTCACCCTGAAAATTAGGCGAGTGGAAGCCGAG |
| | | GACGTCGGAGTGTACTACTGCATGCAGCGCATCGGCTTCCCGAT |
| | | TACGTTTGGACAGGGTACCCGGCTTGAGATCAAG |
| SEQ ID | Linker | GGGGSGGGGSGGGGSGGGGS |
| NO: 63 | | |
| SEQ ID | scFv (VH- | EVQLVESGGGLVKPGGSLRLSCAASGFTFSSFRMNWVRQAPGKGL |
| NO: 175 | linker-VL) | EWVSSISSSSSYIYYADSVKGRFTISRDNAKNSLYLQMNSLRAEDT |
| | | AVYYCARWLSYYGMDVWGQGTTVTVSSGGGGSGGGGSGGGGS |
| | | GGGGSDIVMTQTPLSLPVTPGEPASISCRSSQSLLDSDDGNTYLDW |
| | | YLQKPGQSPQLLIYTLSFRASGVPDRFSGSGSGTDFTLKIRRVEAED |
| | | VGVYYCMQRIGFPITFGQGTRLEIK |
| SEQ ID | DNA scFv | GAAGTGCAACTGGTGGAGAGCGGTGGAGGGCTTGTCAAGCCCG |
| NO: 176 | | GAGGATCGCTGCGGCTGTCCTGTGCTGCGTCCGGGTTCACCTTC |
| | | TCCTCGTTCCGCATGAACTGGGTCAGACAGGCACCGGGAAAGG |
| | | GCCTCGAATGGGTGTCCTCAATCTCATCGTCCTCGTCCTACATC |
| | | TACTACGCCGACTCCGTGAAAGGCCGCTTCACCATCTCCCGGGA |
| | | CAACGCCAAGAACTCGCTGTACCTCCAAATGAATAGCCTCAGG |
| | | GCGGAAGATACTGCTGTGTATTACTGCGCACGCTGGCTTTCCTA |
| | | CTACGGCATGGACGTCTGGGGCCAAGGGACCACTGTGACCGTG |
| | | TCTAGCGGAGGCGGAGGTTCAGGGGGCGGTGGATCAGGCGGAG |
| | | GAGGATCGGGGGGTGGTGGATCGGATATCGTGATGACCCAGAC |
| | | TCCCCTGTCCCTGCCTGTGACTCCCGGAGAACCAGCCTCCATTT |
| | | CCTGCCGGTCCTCCCAGTCCCTGCTGGACAGCGACGACGGCAA |
| | | CACTTACCTGGACTGGTACTTGCAGAAGCCGGGCCAATCGCCTC |
| | | AGCTGCTGATCTATACCCTGTCATTCCGGGCCTCAGGAGTGCCT |
| | | GACCGCTTCTCGGGATCAGGGAGCGGGACCGATTTCACCCTGA |
| | | AAATTAGGCGAGTGGAAGCCGAGGACGTCGGAGTGTACTACTG |
| | | CATGCAGCGCATCGGCTTCCCGATTACGTTTGGACAGGGTACCC |
| | | GGCTTGAGATCAAG |
| SEQ ID | Full CAR | EVQLVESGGGLVKPGGSLRLSCAASGFTFSSFRMNWVRQAPGKGL |
| NO: 177 | amino acid | EWVSSISSSSSYIYYADSVKGRFTISRDNAKNSLYLQMNSLRAEDT |
| | sequence | AVYYCARWLSYYGMDVWGQGTTVTVSSGGGGSGGGGSGGGGS |
| | | GGGGSDIVMTQTPLSLPVTPGEPASISCRSSQSLLDSDDGNTYLDW |
| | | YLQKPGQSPQLLIYTLSFRASGVPDRFSGSGSGTDFTLKIRRVEAED |
| | | VGVYYCMQRIGFPITFGQGTRLEIKTTTPAPRPPTPAPTIASQPLSLR |
| | | PEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKF |
| | | SRSADAPAYQQGQNQLYNELNLGRREEYDVLDKRRGRDPEMGG |
| | | KPRRKNPQEGLYNELQKDKMAEAYSEIGMKGERRRGKGHDGLY |
| | | QGLSTATKDTYDALHMQALPPR |
| SEQ ID | Full CAR | GAAGTGCAACTGGTGGAGAGCGGTGGAGGGCTTGTCAAGCCCG |
| NO: 178 | DNA | GAGGATCGCTGCGGCTGTCCTGTGCTGCGTCCGGGTTCACCTTC |
| | sequence | TCCTCGTTCCGCATGAACTGGGTCAGACAGGCACCGGGAAAGG |
| | | GCCTCGAATGGGTGTCCTCAATCTCATCGTCCTCGTCCTACATC |
| | | TACTACGCCGACTCCGTGAAAGGCCGCTTCACCATCTCCCGGGA |
| | | CAACGCCAAGAACTCGCTGTACCTCCAAATGAATAGCCTCAGG |
| | | GCGGAAGATACTGCTGTGTATTACTGCGCACGCTGGCTTTCCTA |
| | | CTACGGCATGGACGTCTGGGGCCAAGGGACCACTGTGACCGTG |
| | | TCTAGCGGAGGCGGAGGTTCAGGGGGCGGTGGATCAGGCGGAG |
| | | GAGGATCGGGGGGTGGTGGATCGGATATCGTGATGACCCAGAC |
| | | TCCCCTGTCCCTGCCTGTGACTCCCGGAGAACCAGCCTCCATTT |
| | | CCTGCCGGTCCTCCCAGTCCCTGCTGGACAGCGACGACGGCAA |
| | | CACTTACCTGGACTGGTACTTGCAGAAGCCGGGCCAATCGCCTC |
| | | AGCTGCTGATCTATACCCTGTCATTCCGGGCCTCAGGAGTGCCT |
| | | GACCGCTTCTCGGGATCAGGGAGCGGGACCGATTTCACCCTGA |
| | | AAATTAGGCGAGTGGAAGCCGAGGACGTCGGAGTGTACTACTG |
| | | CATGCAGCGCATCGGCTTCCCGATTACGTTTGGACAGGGTACCC |
| | | GGCTTGAGATCAAGACCACTACCCCAGCACCGAGGCCACCCAC |
| | | CCCGGCTCCTACCATCGCCTCCCAGCCTCTGTCCCTGCGTCCGG |
| | | AGGCATGTAGACCCGCAGCTGGTGGGGCCGTGCATACCCGGGG |
| | | TCTTGACTTCGCCTGCGATATCTACATTTGGGCCCCTCTGGCTG |
| | | GTACTTGCGGGGTCCTGCTGCTTTCACTCGTGATCACTCTTTACT |
| | | GTAAGCGCGGTCGGAAGAAGCTGCTGTACATCTTTAAGCAACC |
| | | CTTCATGAGGCCTGTGCAGACTACTCAAGAGGAGGACGGCTGT |
| | | TCATGCCGGTTCCCAGAGGAGGAGGAAGGCGGCTGCGAACTGC |
| | | GCGTGAAATTCAGCCGCAGCGCAGATGCTCCAGCCTACCAGCA |
| | | GGGGCAGAACCAGCTCTACAACGAACTCAATCTTGGTCGGAGA |
| | | GAGGAGTACGACGTGCTGGACAAGCGGAGAGGACGGGACCCA |
| | | GAAATGGGCGGGAAGCCGCGCAGAAAGAATCCCCAAGAGGGC |
| | | CTGTACAACGAGCTCCAAAAGGATAAGATGGCAGAAGCCTATA |
| | | GCGAGATTGGTATGAAAGGGGAACGCAGAAGAGGCAAAGGCC |
| | | ACGACGGACTGTACCAGGGACTCAGCACCGCCACCAAGGACAC |
| | | CTATGACGCTCTTCACATGCAGGCCCTGCCGCCTCGG |

TABLE 13
Kabat CDRs of exemplary hybridoma-derived anti-BCMA molecules

| Kabat | HCDR1 | HCDR2 | HCDR3 | LCDR1 | LCDR2 | LCDR3 |
|---|---|---|---|---|---|---|
| Hy03 | GFWMS | NIKQDGSEK | ALDYYGMD | RSSQSLLDS | TLSYRA | TQRLEFP |
| | (SEQ ID | YYVDSVRG | V (SEQ ID | DDGNTYLD | S (SEQ ID | SIT (SEQ |
| | NO: 137) | (SEQ ID NO: | NO: 139) | (SEQ ID NO: | NO: 148) | ID NO: |
| | | 138) | | 147) | | 149) |
| Hy52 | SFRMN | SISSSSSYIYY | WLSYYGMD | RSSQSLLDS | TLSFRAS | MQRIGFP |
| | (SEQ ID | ADSVKG | V (SEQ ID | DDGNTYLD | (SEQ ID | IT (SEQ |
| | NO: 160) | (SEQ ID NO: | NO: 162) | (SEQ ID NO: | NO: 170) | ID NO: |
| | | 161) | | 147) | | 171) |
| Consensus | X₁FX₂MX₃, | X₁IX₂X₃X₄X₅S | X₁LX₂YYGM | RSSQSLLDS | TLSXRA | X₁QRX₂X₃ |
| | wherein | X₆X₇YYX₈DS | DV, wherein | DDGNTYLD | S, wherein | FPX₄IT, |
| | X₁ is G or | VX₉G, wherein | X₁ is A or W; | (SEQ ID NO: | X is Y or | wherein |
| | S; X₂ is W | X₁ is N or S; | and X₂ is D or | 147) | F (SEQ ID | X₁ is T or |
| | or R; and | X₂ is K or S; | S (SEQ ID | | NO: 182) | M; X₂ is L |
| | X₃ is S or | X₃ is Q or S; | NO: 181) | | | or I; X₃ is |
| | N (SEQ ID | X₄ is D or S; | | | | E or G; |
| | NO: 179) | X₅ is G or S; | | | | and X₄ is S |
| | | X₆ is E or Y; | | | | or absent |
| | | X₇ is K or I; X₈ | | | | (SEQ ID |
| | | is V or A; and | | | | NO: 183) |
| | | X₉ is R or K | | | | |
| | | (SEQ ID NO: | | | | |
| | | 180) | | | | |

TABLE 14
Chothia CDRs of exemplary hybridoma-derived anti-BCMA molecules

| Chothia | HCDR1 | HCDR2 | HCDR3 | LCDR1 | LCDR2 | LCDR3 |
|---|---|---|---|---|---|---|
| Hy03 | GFTFSGF | KQDGSE (SEQ | ALDYYGMD | SQSLLDSD | TLS | RLEFPSI |
| | (SEQ ID | ID NO: 141) | V (SEQ ID | DGNTY | (SEQ ID | (SEQ ID |
| | NO: 140) | | NO: 139) | (SEQ ID | NO: | NO: 152) |
| | | | | NO: 150) | 151) | |
| Hy52 | GFTFSSF | SSSSSY (SEQ | WLSYYGMD | SQSLLDSD | TLS | RIGFPI |
| | (SEQ ID | ID NO: 164) | V (SEQ ID | DGNTY | (SEQ ID | (SEQ ID |
| | NO: 163) | | NO: 162) | (SEQ ID | NO: | NO: 172) |
| | | | | NO: 150) | 151) | |
| Consensus | GFTFSXF, | X₁X₂X₃X₄SX₅, | X₁LX₂YYGM | SQSLLDSD | TLS | RX₁X₂FP |
| | wherein X is | wherein X₁ is K | DV, wherein | DGNTY | (SEQ ID | X₃I, |
| | G or S (SEQ | or S; X₂ is Q or | X₁ is A or W; | (SEQ ID | NO: | wherein |
| | ID NO: 184) | S; X₃ is D or S; | and X₂ is D or | NO: 150) | 151) | X₁ is L or |
| | | X₄ is G or S; and | S (SEQ ID | | | I; X₂ is E |
| | | X₅ is E or Y | NO: 181) | | | or G; and |
| | | (SEQ ID NO: | | | | X₃ is S or |
| | | 185) | | | | absent |
| | | | | | | (SEQ ID |
| | | | | | | NO: 186) |

TABLE 15
IMGT CDRs of exemplary hybridoma-derived anti-BCMA molecules

| IMGT | HCDR1 | HCDR2 | HCDR3 | LCDR1 | LCDR2 | LCDR3 |
|---|---|---|---|---|---|---|
| Hy03 | GFTFSGF | IKQDGSEK | ARALDYYG | QSLLDSDD | TLS | TQRLEFPS |
| | W (SEQ ID | (SEQ ID NO: | MDV (SEQ | GNTY (SEQ | (SEQ ID | IT (SEQ ID |
| | NO: 142) | 143) | ID NO: 144) | ID NO: 153) | NO: 151) | NO: 149) |
| Hy52 | GFTFSSFR | ISSSSSYI | ARWLSYYG | QSLLDSDD | TLS | MQRIGFPI |
| | (SEQ ID | (SEQ ID NO: | MDV (SEQ | GNTY (SEQ | (SEQ ID | T (SEQ ID |
| | NO: 165) | 166) | ID NO: 167) | ID NO: 153) | NO: 151) | NO: 171) |
| Consensus | GFTFSX₁F | IX₁X₂X₃X₄SX₅ | ARX₁LX₂YY | QSLLDSDD | TLS | X₁QRX₂X₃ |
| | X₂, wherein | X₆, wherein | GMDV, | GNTY (SEQ | (SEQ ID | FPX₄IT, |
| | X₁ is G or S; | X₁ is K or S; | wherein X₁ is | ID NO: 153) | NO: 151) | wherein X₁ |
| | and X₂ is W | X₂ is Q or S; | A or W; and | | | is T or M; |
| | or R (SEQ | X₃ is D or S; | X₂ is D or S | | | X₂ is L or I; |
| | ID NO: 187) | X₄ is G or S; | (SEQ ID NO: | | | X₃ is E or |
| | | X₅ is E or Y; | 189) | | | G; and X₄ is |
| | | and X₆ is K or | | | | S or absent |
| | | I (SEQ ID | | | | (SEQ ID |
| | | NO: 188) | | | | NO: 183) |

In some embodiments, the human anti-BCMA binding domain comprises a HC CDR1, HC CDR2, HC CDR3, LC CDR1, LC CDR2, and LC CDR3.

In certain embodiments, the CAR molecule described herein or the anti-BCMA binding domain described herein includes:
(1) one, two, or three light chain (LC) CDRs chosen from:
   (i) a LC CDR1 of SEQ ID NO: 54, LC CDR2 of SEQ ID NO: 55 and LC CDR3 of SEQ ID NO: 56; and/or
(2) one, two, or three heavy chain (HC) CDRs from one of the following:
   (i) a HC CDR1 of SEQ ID NO: 44, HC CDR2 of SEQ ID NO: 45 and HC CDR3 of SEQ ID NO: 84; (ii) a HC CDR1 of SEQ ID NO: 44, HC CDR2 of SEQ ID NO: 45 and HC CDR3 of SEQ ID NO: 46; (iii) a HC CDR1 of SEQ ID NO: 44, HC CDR2 of SEQ ID NO: 45 and HC CDR3 of SEQ ID NO: 68; or (iv) a HC CDR1 of SEQ ID NO: 44, HC CDR2 of SEQ ID NO: 45 and HC CDR3 of SEQ ID NO: 76.

In certain embodiments, the CAR molecule described herein or the anti-BCMA binding domain described herein includes:
(1) one, two, or three light chain (LC) CDRs from one of the following:
   (i) a LC CDR1 of SEQ ID NO: 95, LC CDR2 of SEQ ID NO: 131 and LC CDR3 of SEQ ID NO: 132; (ii) a LC CDR1 of SEQ ID NO: 95, LC CDR2 of SEQ ID NO: 96 and LC CDR3 of SEQ ID NO: 97; (iii) a LC CDR1 of SEQ ID NO: 95, LC CDR2 of SEQ ID NO: 114 and LC CDR3 of SEQ ID NO: 115; or (iv) a LC CDR1 of SEQ ID NO: 95, LC CDR2 of SEQ ID NO: 114 and LC CDR3 of SEQ ID NO: 97; and/or
(2) one, two, or three heavy chain (HC) CDRs from one of the following:
   (i) a HC CDR1 of SEQ ID NO: 86, HC CDR2 of SEQ ID NO: 130 and HC CDR3 of SEQ ID NO: 88; (ii) a HC CDR1 of SEQ ID NO: 86, HC CDR2 of SEQ ID NO: 87 and HC CDR3 of SEQ ID NO: 88; or (iii) a HC CDR1 of SEQ ID NO: 86, HC CDR2 of SEQ ID NO: 109 and HC CDR3 of SEQ ID NO: 88.

In certain embodiments, the CAR molecule described herein or the anti-BCMA binding domain described herein includes:
(1) one, two, or three light chain (LC) CDRs from one of the following:
   (i) a LC CDR1 of SEQ ID NO: 147, LC CDR2 of SEQ ID NO: 182 and LC CDR3 of SEQ ID NO: 183; (ii) a LC CDR1 of SEQ ID NO: 147, LC CDR2 of SEQ ID NO: 148 and LC CDR3 of SEQ ID NO: 149; or (iii) a LC CDR1 of SEQ ID NO: 147, LC CDR2 of SEQ ID NO: 170 and LC CDR3 of SEQ ID NO: 171; and/or
(2) one, two, or three heavy chain (HC) CDRs from one of the following:
   (i) a HC CDR1 of SEQ ID NO: 179, HC CDR2 of SEQ ID NO: 180 and HC CDR3 of SEQ ID NO: 181; (ii) a HC CDR1 of SEQ ID NO: 137, HC CDR2 of SEQ ID NO: 138 and HC CDR3 of SEQ ID NO: 139; or (iii) a HC CDR1 of SEQ ID NO: 160, HC CDR2 of SEQ ID NO: 161 and HC CDR3 of SEQ ID NO: 162.

In some embodiments, the HC CDR1, HC CDR2, HC CDR3, LC CDR1, LC CDR2, and LC CDR3 comprise the amino acid sequences of SEQ ID NOs: 44, 45, 84, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 46, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 68, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 76, 54, 55, and 56, respectively.

In some embodiments, the HC CDR1, HC CDR2, HC CDR3, LC CDR1, LC CDR2, and LC CDR3 comprise the amino acid sequences of SEQ ID NOs: 47, 48, 84, 57, 58, and 59, respectively. In some embodiments, the HC CDR1, HC CDR2, HC CDR3, LC CDR1, LC CDR2, and LC CDR3 comprise the amino acid sequences of SEQ ID NOs: 47, 48, 46, 57, 58, and 59, respectively. In some embodiments, the HC CDR1, HC CDR2, HC CDR3, LC CDR1, LC CDR2, and LC CDR3 comprise the amino acid sequences of SEQ ID NOs: 47, 48, 68, 57, 58, and 59, respectively. In some embodiments, the HC CDR1, HC CDR2, HC CDR3, LC CDR1, LC CDR2, and LC CDR3 comprise the amino acid sequences of SEQ ID NOs: 47, 48, 76, 57, 58, and 59, respectively.

In some embodiments, the HC CDR1, HC CDR2, HC CDR3, LC CDR1, LC CDR2, and LC CDR3 comprise the amino acid sequences of SEQ ID NOs: 49, 50, 85, 60, 58, and 56, respectively. In some embodiments, the HC CDR1, HC CDR2, HC CDR3, LC CDR1, LC CDR2, and LC CDR3 comprise the amino acid sequences of SEQ ID NOs: 49, 50, 51, 60, 58, and 56, respectively. In some embodiments, the HC CDR1, HC CDR2, HC CDR3, LC CDR1, LC CDR2, and LC CDR3 comprise the amino acid sequences of SEQ ID NOs: 49, 50, 69, 60, 58, and 56, respectively. In some embodiments, the HC CDR1, HC CDR2, HC CDR3, LC CDR1, LC CDR2, and LC CDR3 comprise the amino acid sequences of SEQ ID NOs: 49, 50, 77, 60, 58, and 56, respectively.

In some embodiments, the human anti-BCMA binding domain comprises a scFv comprising a VH (for example, a VH described herein) and VL (for example, a VL described herein). In some embodiments, the VH is attached to the VL via a linker, for example, a linker described herein, for example, a linker described in Table 1. In some embodiments, the human anti-BCMA binding domain comprises a (Gly4-Ser) n linker, wherein n is 1, 2, 3, 4, 5, or 6, preferably 3 or 4 (SEQ ID NO: 26). The light chain variable region and heavy chain variable region of a scFv can be, for example, in any of the following orientations: light chain variable region-linker-heavy chain variable region or heavy chain variable region-linker-light chain variable region.

In some embodiments, the anti-BCMA binding domain is a fragment, for example, a single chain variable fragment (scFv). In some embodiments, the anti-BCMA binding domain is a Fv, a Fab, a (Fab′) 2, or a bi-functional (for example bi-specific) hybrid antibody (for example, Lanzavecchia et al., Eur. J. Immunol. 17, 105 (1987)). In some embodiments, the antibodies and fragments thereof of the invention binds a BCMA protein with wild-type or enhanced affinity.

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (for example, a Ser-Gly linker) with an optimized length and/or amino acid composition. The linker length can greatly affect how the variable regions of a scFv fold and interact. In fact, if a short polypeptide linker is employed (for example, between 5-10 amino acids) intrachain folding is prevented. Interchain folding is also required to bring the two variable regions together to form a functional epitope binding site. For examples of linker orientation and size see, for example, Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In some embodiments, the linker sequence comprises sets of glycine and serine repeats such as (Gly₄Ser) n, where n is a positive integer equal to or greater than 1 (SEQ ID NO: 25). In some embodiments, the linker can be (Gly₄Ser)₄ (SEQ ID NO: 27) or (Gly₄Ser)₃ (SEQ ID NO: 28). Variation in the linker length may retain or enhance activity, giving rise to superior efficacy in activity studies.

### CD20 CAR

In some embodiments, the CAR-expressing cell described herein is a CD20 CAR-expressing cell (for example, a cell expressing a CAR that binds to human CD20). In some embodiments, the CD20 CAR-expressing cell includes an antigen binding domain according to WO2016164731 and WO2018067992, incorporated herein by reference. Exemplary CD20-binding sequences or CD20 CAR sequences are disclosed in, for example, Tables 1-5 of WO2018067992. In some embodiments, the CD20 CAR comprises a CDR, variable region, scFv, or full-length sequence of a CD20 CAR disclosed in WO2018067992 or WO2016164731.

### CD22 CAR

In some embodiments, the CAR-expressing cell described herein is a CD22 CAR-expressing cell (for example, a cell expressing a CAR that binds to human CD22). In some embodiments, the CD22 CAR-expressing cell includes an antigen binding domain according to WO2016164731 and WO2018067992, incorporated herein by reference. Exemplary CD22-binding sequences or CD22 CAR sequences are disclosed in, for example, Tables 6A, 6B, 7A, 7B, 7C, 8A, 8B, 9A, 9B, 10A, and 10B of WO2016164731 and Tables 6-10 of WO2018067992. In some embodiments, the CD22 CAR sequences comprise a CDR, variable region, scFv or full-length sequence of a CD22 CAR disclosed in WO2018067992 or WO2016164731.

In embodiments, the CAR molecule comprises an antigen binding domain that binds to CD22 (CD22 CAR). In some embodiments, the antigen binding domain targets human CD22. In some embodiments, the antigen binding domain includes a single chain Fv sequence as described herein.

The sequences of human CD22 CAR are provided below. In some embodiments, a human CD22 CAR is CAR22-65.

Human CD22 CAR scFv sequence
(SEQ ID NO: 285)
EVQLQQSGPGLVKPSQTLSLTCAISGDSMLSNSDTWNWIRQSPSRGLEWL
GRTYHRSTWYDDYASSVRGRVSINVDTSKNQYSLQLNAVTPEDTGVYYCA
RVRLQDGNSWSDAFDVWGQGTMVTVSSGGGGSGGGGSGGGGSQSALTQPA
SASGSPGQSVTISCTGTSSDVGGYNYVSWYQQHPGKAPKLMIYDVSNRPS
GVSNRFSGSKSGNTASLTISGLQAEDEADYYCSSYTSSSTLYVFGTGTQL
TVL
Human CD22 CAR heavy chain variable region
(SEQ ID NO 286)
EVQLQQSGPGLVKPSQTLSLTCAISGDSMLSNSDTWNWIRQSPSRGLEWL
GRTYHRSTWYDDYASSVRGRVSINVDTSKNQYSLQLNAVTPEDTGVYYCA
RVRLQDGNSWSDAFDVWGQGTMVTVSS
Human CD22 CAR light chain variable region
(SEQ ID NO 287)
QSALTQPASASGSPGQSVTISCTGTSSDVGGYNYVSWYQQHPGKAPKLMI
YDVSNRPSGVSNRFSGSKSGNTASLTISGLQAEDEADYYCSSYTSSSTLY
VFGTGTQLTVL

TABLE 16
Heavy Chain Variable Domain CDRs of CD22 CAR
(CAR22-65)

| | | SEQ | | SEQ | | SEQ |
|---|---|---|---|---|---|---|
| | | ID | | ID | | ID |
| Candidate | HCDR1 | NO: | HCDR2 | NO: | HCDR3 | NO: |
| CAR22-65 | GDSML | 288 | RTYHRSTWYDDYA | 290 | VRLQDGNSWSD | 291 |
| Combined | SNSDT | | SSVRG | | AFDV | |
| | WN | | | | | |
| CAR22-65 | SNSDT | 289 | RTYHRSTWYDDYA | 290 | VRLQDGNSWSD | 291 |
| Kabat | WN | | SSVRG | | AFDV | |

TABLE 17
Light Chain Variable Domain CDRs of CD22 CAR
(CAR22-65). The LC CDR sequences in this table
have the same sequence under the Kabat or
combined definitions.

| | | SEQ | | SEQ | | SEQ |
|---|---|---|---|---|---|---|
| | | ID | | ID | | ID |
| Candidate | LCDR1 | NO: | LCDR2 | NO: | LCDR3 | NO: |
| CAR22-65 | TGTSSDV | 95 | DVSNRPS | 96 | SSYTSSSTLYV | 97 |
| Combined | GGYNYVS | | | | | |

In some embodiments, the antigen binding domain comprises a HC CDR1, a HC CDR2, and a HC CDR3 of any heavy chain binding domain amino acid sequences listed in Table 16. In embodiments, the antigen binding domain further comprises a LC CDR1, a LC CDR2, and a LC CDR3. In embodiments, the antigen binding domain comprises a LC CDR1, a LC CDR2, and a LC CDR3 amino acid sequences listed in Table 17.

In some embodiments, the antigen binding domain comprises one, two or all of LC CDR1, LC CDR2, and LC CDR3 of any light chain binding domain amino acid sequences listed in Table 17, and one, two or all of HC CDR1, HC CDR2, and HC CDR3 of any heavy chain binding domain amino acid sequences listed in Table 16.

In some embodiments, the CDRs are defined according to the Kabat numbering scheme, the Chothia numbering scheme, or a combination thereof.

The order in which the VL and VH domains appear in the scFv can be varied (i.e., VL-VH, or VH-VL orientation), and where any of one, two, three or four copies of the “G4S” subunit (SEQ ID NO: 25), in which each subunit comprises the sequence GGGGS (SEQ ID NO: 25) (for example, (G4S)₃ (SEQ ID NO: 28) or (G4S)₄ (SEQ ID NO: 27)), can connect the variable domains to create the entirety of the scFv domain. Alternatively, the CAR construct can include, for example, a linker including the sequence GSTSGSGKPGSGEGSTKG (SEQ ID NO: 43). Alternatively, the CAR construct can include, for example, a linker including the sequence LAEAAAK (SEQ ID NO: 308). In some embodiments, the CAR construct does not include a linker between the VL and VH domains.

These clones all contained a Q/K residue change in the signal domain of the co-stimulatory domain derived from CD3zeta chain.

### EGFR CAR

In some embodiments, the CAR-expressing cell described herein is an EGFR CAR-expressing cell (for example, a cell expressing a CAR that binds to human EGFR). In some embodiments, the CAR-expressing cell described herein is an EGFRvIII CAR-expressing cell (for example, a cell expressing a CAR that binds to human EGFRvIII). Exemplary EGFRvIII CARs can include sequences disclosed in WO2014/130657, for example, Table 2 of WO2014/130657, incorporated herein by reference.

Exemplary EGFRvIII-binding sequences or EGFR CAR sequences may comprise a CDR, a variable region, an scFv, or a full-length CAR sequence of a EGFR CAR disclosed in WO2014/130657.

### Mesothelin CAR

In some embodiments, the CAR-expressing cell described herein is a mesothelin CAR-expressing cell (for example, a cell expressing a CAR that binds to human mesothelin). Exemplary mesothelin CARs can include sequences disclosed in WO2015090230 and WO2017112741, for example, Tables 2, 3, 4, and 5 of WO2017112741, incorporated herein by reference.

### Other Exemplary CARs

In other embodiments, the CAR-expressing cells can specifically bind to CD123, for example, can include a CAR molecule (for example, any of the CAR1 to CAR8), or an antigen binding domain according to Tables 1-2 of WO 2014/130635, incorporated herein by reference. The amino acid and nucleotide sequences encoding the CD123 CAR molecules and antigen binding domains (for example, including one, two, three VH CDRs; and one, two, three VL CDRs according to Kabat or Chothia), are specified in WO 2014/130635. In other embodiments, the CAR-expressing cells can specifically bind to CD123, for example, can include a CAR molecule (for example, any of the CAR123-1 to CAR123-4 and hzCAR123-1 to hzCAR123-32), or an antigen binding domain according to Tables 2, 6, and 9 of WO2016/028896, incorporated herein by reference. The amino acid and nucleotide sequences encoding the CD123 CAR molecules and antigen binding domains (for example, including one, two, three VH CDRs; and one, two, three VL CDRs according to Kabat or Chothia), are specified in WO2016/028896.

In some embodiments, the CAR molecule comprises a CLL1 CAR described herein, for example, a CLL1 CAR described in US2016/0051651A1, incorporated herein by reference. In embodiments, the CLL1 CAR comprises an amino acid, or has a nucleotide sequence shown in US2016/0051651A1, incorporated herein by reference. In other embodiments, the CAR-expressing cells can specifically bind to CLL-1, for example, can include a CAR molecule, or an antigen binding domain according to Table 2 of WO2016/014535, incorporated herein by reference. The amino acid and nucleotide sequences encoding the CLL-1 CAR molecules and antigen binding domains (for example, including one, two, three VH CDRs; and one, two, three VL CDRs according to Kabat or Chothia), are specified in WO2016/014535.

In some embodiments, the CAR molecule comprises a CD33 CAR described herein, e.ga CD33 CAR described in US2016/0096892A1, incorporated herein by reference. In embodiments, the CD33 CAR comprises an amino acid, or has a nucleotide sequence shown in US2016/0096892A1, incorporated herein by reference. In other embodiments, the CAR-expressing cells can specifically bind to CD33, for example, can include a CAR molecule (for example, any of CAR33-1 to CAR-33-9), or an antigen binding domain according to Table 2 or 9 of WO2016/014576, incorporated herein by reference. The amino acid and nucleotide sequences encoding the CD33 CAR molecules and antigen binding domains (for example, including one, two, three VH CDRs; and one, two, three VL CDRs according to Kabat or Chothia), are specified in WO2016/014576.

In some embodiments, the antigen binding domain comprises one, two three (for example, all three) heavy chain CDRs, HC CDR1, HC CDR2 and HC CDR3, from an antibody described herein (for example, an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference), and/or one, two, three (for example, all three) light chain CDRs, LC CDR1, LC CDR2 and LC CDR3, from an antibody described herein (for example, an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference). In some embodiments, the antigen binding domain comprises a heavy chain variable region and/or a variable light chain region of an antibody listed above.

In embodiments, the antigen binding domain is an antigen binding domain described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference.

In embodiments, the antigen binding domain targets BCMA and is described in US-2016-0046724-A1. In embodiments, the antigen binding domain targets CD19 and is described in US-2015-0283178-A1. In embodiments, the antigen binding domain targets CD123 and is described in US2014/0322212A1, US2016/0068601A1. In embodiments, the antigen binding domain targets CLL1 and is described in US2016/0051651A1. In embodiments, the antigen binding domain targets CD33 and is described in US2016/0096892A1.

Exemplary target antigens that can be targeted using the CAR-expressing cells, include, but are not limited to, CD19, CD123, EGFRVIII, CD33, mesothelin, BCMA, and GFR ALPHA-4, among others, as described in, for example, WO2014/153270, WO 2014/130635, WO2016/028896, WO 2014/130657, WO2016/014576, WO 2015/090230, WO2016/014565, WO2016/014535, and WO2016/025880, each of which is herein incorporated by reference in its entirety.

In other embodiments, the CAR-expressing cells can specifically bind to GFR ALPHA-4, for example, can include a CAR molecule, or an antigen binding domain according to Table 2 of WO2016/025880, incorporated herein by reference. The amino acid and nucleotide sequences encoding the GFR ALPHA-4 CAR molecules and antigen binding domains (for example, including one, two, three VH CDRs; and one, two, three VL CDRs according to Kabat or Chothia), are specified in WO2016/025880.

In some embodiments, the antigen binding domain of any of the CAR molecules described herein (for example, any of CD19, CD123, EGFRVIII, CD33, mesothelin, BCMA, and GFR ALPHA-4) comprises one, two three (for example, all three) heavy chain CDRs, HC CDR1, HC CDR2 and HC CDR3, from an antibody listed above, and/or one, two, three (for example, all three) light chain CDRs, LC CDR1, LC CDR2 and LC CDR3, from an antigen binding domain listed above. In some embodiments, the antigen binding domain comprises a heavy chain variable region and/or a variable light chain region of an antibody listed or described above.

In some embodiments, the antigen binding domain comprises one, two three (for example, all three) heavy chain CDRs, HC CDR1, HC CDR2 and HC CDR3, from an antibody listed above, and/or one, two, three (for example, all three) light chain CDRs, LC CDR1, LC CDR2 and LC CDR3, from an antibody listed above. In some embodiments, the antigen binding domain comprises a heavy chain variable region and/or a variable light chain region of an antibody listed or described above.

In some embodiments, the tumor antigen is a tumor antigen described in International Application WO2015/142675, filed Mar. 13, 2015, which is herein incorporated by reference in its entirety. In some embodiments, the tumor antigen is chosen from one or more of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1) Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In some embodiments, the antigen binding domain comprises one, two three (for example, all three) heavy chain CDRs, HC CDR1, HC CDR2 and HC CDR3, from an antibody listed above, and/or one, two, three (for example, all three) light chain CDRs, LC CDR1, LC CDR2 and LC CDR3, from an antibody listed above. In some embodiments, the antigen binding domain comprises a heavy chain variable region and/or a variable light chain region of an antibody listed or described above.

In some embodiments, the anti-tumor antigen binding domain is a fragment, for example, a single chain variable fragment (scFv). In some embodiments, the anti-a cancer associate antigen as described herein binding domain is a Fv, a Fab, a (Fab′) 2, or a bi-functional (for example bi-specific) hybrid antibody (for example, Lanzavecchia et al., Eur. J. Immunol. 17, 105 (1987)). In some embodiments, the antibodies and fragments thereof of the invention binds a cancer associate antigen as described herein protein with wild-type or enhanced affinity.

In some instances, scFvs can be prepared according to a method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (for example, a Ser-Gly linker) with an optimized length and/or amino acid composition. The linker length can greatly affect how the variable regions of a scFv fold and interact. In fact, if a short polypeptide linker is employed (for example, between 5-10 amino acids) intrachain folding is prevented. Interchain folding is also required to bring the two variable regions together to form a functional epitope binding site. For examples of linker orientation and size see, for example, Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, which are incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In some embodiments, the linker sequence comprises sets of glycine and serine repeats such as (Gly₄Ser)n, where n is a positive integer equal to or greater than 1 (SEQ ID NO: 25). In some embodiments, the linker can be (Gly₄Ser)₄ (SEQ ID NO: 27) or (Gly₄Ser)₃ (SEQ ID NO: 28). Variation in the linker length may retain or enhance activity, giving rise to superior efficacy in activity studies.

In some embodiments, the antigen binding domain is a T cell receptor (“TCR”), or a fragment thereof, for example, a single chain TCR (scTCR). Methods to make such TCRs are known in the art. See, for example, Willemsen R A et al, Gene Therapy 7:1369-1377 (2000); Zhang T et al, Cancer Gene Ther 11:487-496 (2004); Aggen et al, Gene Ther. 19 (4): 365-74 (2012) (references are incorporated herein by its entirety). For example, scTCR can be engineered that contains the Vα and Vβ genes from a T cell clone linked by a linker (for example, a flexible peptide). This approach is very useful to cancer associated target that itself is intracellular, however, a fragment of such antigen (peptide) is presented on the surface of the cancer cells by MHC.

### Transmembrane Domain

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, for example, one or more amino acid associated with the extracellular region of the protein from which the transmembrane was derived (for example, 1, 2, 3, 4, 5, 6, 7, 8, 9, 10 up to 15 amino acids of the extracellular region) and/or one or more additional amino acids associated with the intracellular region of the protein from which the transmembrane protein is derived (for example, 1, 2, 3, 4, 5, 6, 7, 8, 9, 10 up to 15 amino acids of the intracellular region). In some embodiments, the transmembrane domain is one that is associated with one of the other domains of the CAR is used. In some instances, the transmembrane domain can be selected or modified by amino acid substitution to avoid binding of such domains to the transmembrane domains of the same or different surface membrane proteins, for example, to minimize interactions with other members of the receptor complex. In some embodiments, the transmembrane domain is capable of homodimerization with another CAR on the CAR-expressing cell, for example, CART cell, surface. In some embodiments the amino acid sequence of the transmembrane domain may be modified or substituted so as to minimize interactions with the binding domains of the native binding partner present in the same CAR-expressing cell, for example, CART.

The transmembrane domain may be derived either from a natural or from a recombinant source. Where the source is natural, the domain may be derived from any membrane-bound or transmembrane protein. In some embodiments the transmembrane domain is capable of signaling to the intracellular domain(s) whenever the CAR has bound to a target. A transmembrane domain of particular use in this invention may include at least the transmembrane region(s) of, for example, the alpha, beta or zeta chain of T-cell receptor, CD28, CD3 epsilon, CD45, CD4, CD5, CD8 (for example, CD8 alpha, CD8 beta), CD9, CD16, CD22, CD33, CD37, CD64, CD80, CD86, CD134, CD137, CD154. In some embodiments, a transmembrane domain may include at least the transmembrane region(s) of a costimulatory molecule, for example, MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, 30) SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In some instances, the transmembrane domain can be attached to the extracellular region of the CAR, for example, the antigen binding domain of the CAR, via a hinge, for example, a hinge from a human protein. For example, in some embodiments, the hinge can be a human Ig (immunoglobulin) hinge, for example, an IgG4 hinge, or a CD8a hinge. In some embodiments, the hinge or spacer comprises (for example, consists of) the amino acid sequence of SEQ ID NO: 2. In some embodiments, the transmembrane domain comprises (for example, consists of) a transmembrane domain of SEQ ID NO: 6.

In some embodiments, the hinge or spacer comprises an IgG4 hinge. For example, in some embodiments, the hinge or spacer comprises a hinge of SEQ ID NO: 3. In some embodiments, the hinge or spacer comprises a hinge encoded by the nucleotide sequence of SEQ ID NO: 14.

In some embodiments, the hinge or spacer comprises an IgD hinge. For example, in some embodiments, the hinge or spacer comprises a hinge of the amino acid sequence of SEQ ID NO: 4. In some embodiments, the hinge or spacer comprises a hinge encoded by the nucleotide sequence of SEQ ID NO: 15.

In some embodiments, the transmembrane domain may be recombinant, in which case it will comprise predominantly hydrophobic residues such as leucine and valine. In some embodiments, a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in some embodiments, the linker comprises the amino acid sequence of SEQ ID NO: 5. In some embodiments, the linker is encoded by a nucleotide sequence of SEQ ID NO: 16.

In some embodiments, the hinge or spacer comprises a KIR2DS2 hinge.

### Cytoplasmic Domain

The cytoplasmic domain or region of a CAR of the present invention includes an intracellular signaling domain. An intracellular signaling domain is generally responsible for activation of at least one of the normal effector functions of the immune cell in which the CAR has been introduced.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, for example, a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the invention include those of TCR zeta, FcR gamma, FcR beta, CD3 gamma, CD3 delta, CD3 epsilon, CD5, CD22, CD79a, CD79b, CD278 (also known as “ICOS”), FcεRI, DAP10, DAP12, and CD66d. In some embodiments, a CAR of the invention comprises an intracellular signaling domain, for example, a primary signaling domain of CD3-zeta.

In some embodiments, a primary signaling domain comprises a modified ITAM domain, for example, a mutated ITAM domain which has altered (for example, increased or decreased) activity as compared to the native ITAM domain. In some embodiments, a primary signaling domain comprises a modified ITAM-containing primary intracellular signaling domain, for example, an optimized and/or truncated ITAM-containing primary intracellular signaling domain. In some embodiments, a primary signaling domain comprises one, two, three, four or more ITAM motifs.

Further examples of molecules containing a primary intracellular signaling domain that are of particular use in the invention include those of DAP10, DAP12, and CD32.

The intracellular signaling domain of the CAR can comprise the primary signaling domain, for example, CD3-zeta signaling domain, by itself or it can be combined with any other desired intracellular signaling domain(s) useful in the context of a CAR of the invention. For example, the intracellular signaling domain of the CAR can comprise a primary signaling domain, for example, CD3 zeta chain portion, and a costimulatory signaling domain. The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119 (3): 696-706). The intracellular signaling sequences within the cytoplasmic portion of the CAR of the invention may be linked to each other in a random or specified order. Optionally, a short oligo- or polypeptide linker, for example, between 2 and 10 amino acids (for example, 2, 3, 4, 5, 6, 7, 8, 9, or 10 amino acids) in length may form the linkage between intracellular signaling sequence. In some embodiments, a glycine-serine doublet can be used as a suitable linker. In some embodiments, a single amino acid, for example, an alanine, a glycine, can be used as a suitable linker.

In some embodiments, the intracellular signaling domain is designed to comprise two or more, for example, 2, 3, 4, 5, or more, costimulatory signaling domains. In some embodiments, the two or more, for example, 2, 3, 4, 5, or more, costimulatory signaling domains, are separated by a linker molecule, for example, a linker molecule described herein. In some embodiments, the intracellular signaling domain comprises two costimulatory signaling domains. In some embodiments, the linker molecule is a glycine residue. In some embodiments, the linker is an alanine residue.

In some embodiments, the intracellular signaling domain is designed to comprise the signaling domain of CD3-zeta and the signaling domain of CD28. In some embodiments, the intracellular signaling domain is designed to comprise the signaling domain of CD3-zeta and the signaling domain of 4-1BB. In some embodiments, the signaling domain of 4-1BB is a signaling domain of SEQ ID NO: 7. In some embodiments, the signaling domain of CD3-zeta is a signaling domain of SEQ ID NO: 9 (mutant CD3zeta) or SEQ ID NO: 10 (wild type human CD3zeta).

In some embodiments, the intracellular signaling domain is designed to comprise the signaling domain of CD3-zeta and the signaling domain of CD27. In some embodiments, the signaling domain of CD27 comprises the amino acid sequence of SEQ ID NO: 8. In some embodiments, the signaling domain of CD27 is encoded by the nucleic acid sequence of SEQ ID NO: 19.

In some embodiments, the intracellular is designed to comprise the signaling domain of CD3-zeta and the signaling domain of CD28. In some embodiments, the signaling domain of CD28 comprises the amino acid sequence of SEQ ID NO: 36. In some embodiments, the signaling domain of CD28 is encoded by the nucleic acid sequence of SEQ ID NO: 37. In some embodiments, the intracellular is designed to comprise the signaling domain of CD3-zeta and the signaling domain of ICOS. In some embodiments, the signaling domain of ICOS comprises the amino acid sequence of SEQ ID NO: 38. In some embodiments, the signaling domain of ICOS is encoded by the nucleic acid sequence of SEQ ID NO: 39.

Co-Expression of CAR with Other Molecules or Agents

### Co-Expression of a Second CAR

In some embodiments, the CAR-expressing cell described herein can further comprise a second CAR, for example, a second CAR that includes a different antigen binding domain, for example, to the same target (for example, CD19) or a different target (for example, a target other than CD19, for example, a target described herein).

In some embodiments, the CAR-expressing cell described herein, e.g., the CAR-expressing cell manufactured using a method described herein, comprises (i) a first nucleic acid molecule encoding a first CAR that binds to BCMA and (ii) a second nucleic acid molecule encoding a second CAR that binds to CD19. In some embodiments, the first CAR comprises an anti-BCMA binding domain, a first transmembrane domain, and a first intracellular signaling domain, wherein the anti-BCMA binding domain comprises a heavy chain variable region (VH) comprising a heavy chain complementary determining region 1 (HC CDR1), a heavy chain complementary determining region 2 (HC CDR2), and a heavy chain complementary determining region 3 (HC CDR3), and a light chain variable region (VL) comprising a light chain complementary determining region 1 (LC CDR1), a light chain complementary determining region 2 (LC CDR2), and a light chain complementary determining region 3 (LC CDR3), wherein the HC CDR1, HC CDR2, HC CDR3, LC CDR1, LC CDR2, and LC CDR3 comprise the amino acid sequences of SEQ ID NOs: 86, 87, 88, 95, 96, and 97, respectively. In some embodiments, the second CAR comprises an anti-CD19 binding domain, a second transmembrane domain, and a second intracellular signaling domain, wherein the anti-CD19 binding domain comprises a VH comprising a HC CDR1, a HC CDR2, and a HC CDR3, and a VL comprising a LC CDR1, a LC CDR2, and a LC CDR3, wherein the HC CDR1, HC CDR2, HC CDR3, LC CDR1, LC CDR2, and LC CDR3 comprise the amino acid sequences of SEQ ID NOs: 295, 304, and 297-300, respectively. In some embodiments, (i) the VH and VL of the anti-BCMA binding domain comprise the amino acid sequences of SEQ ID NOs: 93 and 102, respectively. In some embodiments, the VH and VL of the anti-CD19 binding domain comprise the amino acid sequences of SEQ ID NOs: 250 and 251, respectively. In some embodiments, the anti-BCMA binding domain comprises the amino acid sequence of SEQ ID NO: 105. In some embodiments, the anti-CD19 binding domain comprises the amino acid sequence of SEQ ID NO: 293. In some embodiments, the first CAR comprises the amino acid sequence of SEQ ID NO: 107. In some embodiments, the second CAR comprise the amino acid sequence of SEQ ID NO: 225.

In some embodiments, the CAR-expressing cell described herein, e.g., the CAR-expressing cell manufactured using a method described herein, comprises (i) a first nucleic acid molecule encoding a first CAR that binds to CD22 and (ii) a second nucleic acid molecule encoding a second CAR that binds to CD19. In some embodiments, the CD22 CAR comprises a CD22 antigen binding domain, and a first transmembrane domain; a first co-stimulatory signaling domain; and/or a first primary signaling domain. In some embodiments, the CD19 CAR comprises a CD19 antigen binding domain, and a second transmembrane domain; a second co-stimulatory signaling domain; and/or a second primary signaling domain.

In some embodiments, the CD22 antigen binding domain comprises one or more (e.g., all three) light chain complementarity determining region 1 (LC CDR1), light chain complementarity determining region 2 (LC CDR2), and light chain complementarity determining region 3 (LC CDR3) of a CD22 binding domain described herein, e.g., in Tables 16, 17, 30, 31, or 32; and/or one or more (e.g., all three) heavy chain complementarity determining region 1 (HC CDR1), heavy chain complementarity determining region 2 (HC CDR2), and heavy chain complementarity determining region 3 (HC CDR3) of a CD22 binding domain described herein, e.g., in Tables 16, 17, 30, 31 or 32. In an embodiment, the CD22 antigen binding domain comprises a LC CDR1, LC CDR2 and LC CDR3 of a CD22 binding domain described herein, e.g., in Table 16, 17, 30, 31 or 33; and/or a HC CDR1, HC CDR2 and HC CDR3 of a CD22 binding domain described herein, e.g., in Tables 16, 17, 30, 31 or 33. In some embodiments, the CD19 antigen binding domain comprises: one or more (e.g., all three) LC CDR1, LC CDR2, and LC CDR3 of a CD19 binding domain described herein, e.g., in Tables 2, 30, 31, or 32; and/or one or more (e.g., all three) HC CDR1, HC CDR2, and HC CDR3 of a CD19 binding domain described herein, e.g., in Tables 2, 30, 31, and 32. In some embodiments, the CD19 antigen binding domain comprises a LC CDR1, LC CDR2 and LC CDR3 of a CD19 binding domain described herein, e.g., in Tables 2, 30, 31, and 32; and/or a HC CDR1, HC CDR2 and HC CDR3 of a CD19 binding domain described herein, e.g., in Tables 2, 30, 31, and 32.

In some embodiment, the CD22 antigen binding domain (e.g., an scFv) comprises a light chain variable (VL) region of a CD22 binding domain described herein, e.g., in Tables 30 or 32; and/or a heavy chain variable (VH) region of a CD22 binding domain described herein, e.g., in Tables 30 or 32. In some embodiments, the CD22 antigen binding domain comprises a VL region comprising an amino acid sequence having at least one, two or three modifications (e.g., substitutions) but not more than 30, 20 or 10 modifications (e.g., substitutions) of a CD22 VL region sequence provided in Table 30 or 32. In some embodiments, the CD22 antigen binding domain comprises a VL region comprising an amino acid sequence provided in Table 30 or 32, or a sequence having at least about 80%, 85%, 90%, 92%, 95%, 97%, 98%, or 99% sequence identity to any of the aforesaid sequences. In some embodiments, the CD22 antigen binding domain comprises a VH region comprising an amino acid sequence having at least one, two or three modifications (e.g., substitutions) but not more than 30, 20 or 10 modifications (e.g., substitutions) of a CD22 VH region sequence provided in Table 30 or 32. In some embodiments, the CD22 antigen binding domain comprises a VH region comprising the amino acid sequence of a CD22 VH region sequence provided in Table 30 or 32, or a sequence having at least about 80%, 85%, 90%, 92%, 95%, 97%, 98%, or 99% sequence identity to any of the aforesaid sequences. In some embodiments, the CD19 antigen binding domain (e.g., an scFv) comprises a VL region of a CD19 binding domain described herein, e.g., in Tables 2, 30, or 32; and/or a VH region of a CD19 binding domain described herein, e.g., in Tables 2, 30, or 32. In some embodiments, the CD19 antigen binding domain comprises a VL region comprising an amino acid sequence having at least one, two or three modifications (e.g., substitutions) but not more than 30, 20 or 10 modifications (e.g., substitutions) of a CD19 VL region sequence provided in Tables 2, 30, or 32. In some embodiments, the CD19 antigen binding domain comprises a VL region comprising the amino acid sequence of a CD19 VL region sequence provided in Tables 2, 30, or 32, or a sequence having at least about 80%, 85%, 90%, 92%, 95%, 97%, 98%, or 99% sequence identity to any of the aforesaid sequences. In some embodiments, the CD19 antigen binding domain comprises a VH region comprising an amino acid sequence having at least one, two or three modifications (e.g., substitutions) but not more than 30, 20 or 10 modifications (e.g., substitutions) of a CD19 VH region sequence provided in Tables 2, 30, or 32. In some embodiments, the CD19 antigen binding domain comprises a VH region comprising the amino acid sequence of a CD19 VH region sequence provided in Tables 2, 30, or 32, or a sequence having at least about 80%, 85%, 90%, 92%, 95%, 97%, 98%, or 99% sequence identity to any of the aforesaid sequences.

In some embodiments, the CD22 antigen binding comprises an scFv comprising an amino acid sequence having at least one, two or three modifications (e.g., substitutions) but not more than 30, 20 or 10 modifications (e.g., substitutions) of a CD22 scFv sequence provided in Table 30 or 32. In some embodiments, the CD22 antigen binding comprises an scFv comprising an amino acid sequence of a CD22 scFv sequence provided in Table 30 or 32, or a sequence having at least about 80%, 85%, 90%, 92%, 95%, 97%, 98%, or 99% sequence identity to any of the aforesaid sequences. In some embodiments, the CD19 antigen binding domain comprises an scFv comprising an amino acid sequence having at least one, two or three modifications (e.g., substitutions) but not more than 30, 20 or 10 modifications (e.g., substitutions) of a CD19 scFv sequence provided in Tables 2, 30, or 32. In some embodiments, the CD19 antigen binding domain comprises an scFv comprising the amino acid sequence of a CD19 scFv sequence provided in Tables 2, 30, or 32, or a sequence having at least about 80%, 85%, 90%, 92%, 95%, 97%, 98%, or 99% sequence identity to any of the aforesaid sequences.

In some embodiments, the CD22 CAR molecule and/or the CD19 CAR molecule comprises an additional component, e.g., a signal peptide, a hinge, a transmembrane domain, a co-stimulatory signaling domain and/or a first primary signaling domain, a P2A site, and/or a linker, comprising an amino acid sequence provided in Table 33, or a sequence having at least about 70%, 75%, 80%, 85%, 90%, 92%, 95%, 97%, 98%, or 99% sequence identity to any of the aforesaid sequences; or is encoded by a nucleotide sequence provided in Table 33, or a sequence having at least about 70%, 75%, 80%, 85%, 90%, 92%, 95%, 97%, 98%, or 99% sequence identity to any of the aforesaid sequences.

Exemplary nucleotide and amino acid sequences of a CAR molecule, e.g., a dual CAR molecule comprising (i) a first CAR that binds to CD22 and (ii) a second CAR that binds to CD19 disclosed herein, is provided in Table 30.

TABLE 30
Dual and tandem CD19-CD22 CAR sequences

| | | |
|---|---|---|
| | SEQ ID | |
| Identifier | NO | Sequence |

Tandem CD19-CD22 CARS

| | | |
|---|---|---|
| CG#c171 | 725 | atggccctccctgtcaccgccctgctgcttccgctggctcttctgctccacgccgctcggcccg |
| | | aaattgtgatgacccagtcacccgccactcttagcctttcacccggtgagcgcgcaaccctgtc |
| | | ttgcagagcctcccaagacatctcaaaataccttaattggtatcaacagaagcccggacaggc |
| | | tcctcgccttctgatctaccacaccagccggctccattctggaatccctgccaggttcagcggt |
| | | agcggatctgggaccgactacaccctcactatcagctcactgcagccagaggacttcgctgtc |
| | | tatttctgtcagcaagggaacaccctgccctacacctttggacagggcaccaagctcgagatt |
| | | aaaggtggaggtggcagcggaggaggtgggtccggcggtggaggaagccaggtccaact |
| | | ccaagaaagcggaccgggtcttgtgaagccatcagaaactctttcactgacttgtactgtgagc |
| | | ggagtgtctctccccgattacggggtgtcttggatcagacagccaccggggaagggtctgga |
| | | atggattggagtgatttggggctctgagactacttactaccaatcatccctcaagtcacgcgtca |
| | | ccatctcaaaggacaactctaagaatcaggtgtcactgaaactgtcatctgtgaccgcagccg |
| | | acaccgccgtgtactattgcgctaagcattactattatggcgggagctacgcaatggattactg |
| | | gggacagggtactctggtcaccgtgtccagcttggcagaagccgccgcgaaagaagtgcag |
| | | cttcaacaatcaggaccaggactcgtcaaaccatcacagaccctctccctcacatgtgccatct |
| | | ccggggactccatgttgagcaattccgacacttggaattggattagacaaagcccgtcccggg |
| | | gtctggaatggttgggacgcacctaccaccggtctacttggtacgacgactacgcgtcatccg |
| | | tgcggggaagagtgtccatcaacgtggacacctccaagaaccagtacagcctgcagcttaat |
| | | gccgtgactcctgaggatacgggcgtctactactgcgcccgcgtccgcctgcaagacggga |
| | | acagctggagcgatgcattcgatgtctggggccagggaactatggtcaccgtgtcgtctggg |
| | | ggcggtggatcgggtggcgggggttcggggggggcggctctcagtccgctcttacccaac |
| | | cggcctcagcctcggggagccccggccagagcgtgaccatttcctgcaccggcacttcatcc |
| | | gacgtgggcggctacaactacgtgtcctggtaccaacagcacccgggaaaggcccccaag |
| | | ctcatgatctacgacgtgtccaacaggccctcgggagtgtccaaccggttctcgggttcgaaa |
| | | tcgggaaacacagccagcctgaccatcagcggactgcaggctgaagatgaagccgactact |
| | | actgctcctcctacacctcgtcatccacgctctacgtgttcggcactggaactcagctgactgtg |
| | | ctgaccactaccccagcaccgaggccacccaccccggctcctaccatcgcctcccagcctct |
| | | gtccctgcgtccggaggcatgtagacccgcagctggtggggccgtgcatacccggggtctt |
| | | gacttcgcctgcgatatctacatttgggcccctctggctggtacttgcggggtcctgctgctttca |
| | | ctcgtgatcactctttactgtaagcgcggtcggaagaagctgctgtacatctttaagcaaccctt |
| | | catgaggcctgtgcagactactcaagaggaggacggctgttcatgccggttcccagaggag |
| | | gaggaaggcggctgcgaactgcgcgtgaaattcagccgcagcgcagatgctccagcctac |
| | | cagcaggggcagaaccagctctacaacgaactcaatcttggtcggagagaggagtacgacg |
| | | tgctggacaagcggagaggacgggacccagaaatgggcgggaagccgcgcagaaagaa |
| | | tccccaagagggcctgtacaacgagctccaaaaggataagatggcagaagcctatagcgag |
| | | attggtatgaaaggggaacgcagaagaggcaaaggccacgacggactgtaccagggactc |
| | | agcaccgccaccaaggacacctatgacgctcttcacatgcaggccctgccgcctcgg |
| | 726 | MALPVTALLLPLALLLHAARPEIVMTQSPATLSLSPGERAT |
| | | LSCRASQDISKYLNWYQQKPGQAPRLLIYHTSRLHSGIPAR |
| | | FSGSGSGTDYTLTISSLQPEDFAVYFCQQGNTLPYTFGQGT |
| | | KLEIKGGGGSGGGGSGGGGSQVQLQESGPGLVKPSETLSLT |
| | | CTVSGVSLPDYGVSWIRQPPGKGLEWIGVIWGSETTYYQSS |
| | | LKSRVTISKDNSKNQVSLKLSSVTAADTAVYYCAKHYYYG |
| | | GSYAMDYWGQGTLVTVSSLAEAAAKEVQLQQSGPGLVKP |
| | | SQTLSLTCAISGDSMLSNSDTWNWIRQSPSRGLEWLGRTY |
| | | HRSTWYDDYASSVRGRVSINVDTSKNQYSLQLNAVTPEDT |
| | | GVYYCARVRLQDGNSWSDAFDVWGQGTMVTVSSGGGGS |
| | | GGGGSGGGGSQSALTQPASASGSPGQSVTISCTGTSSDVGG |
| | | YNYVSWYQQHPGKAPKLMIYDVSNRPSGVSNRFSGSKSG |
| | | NTASLTISGLQAEDEADYYCSSYTSSSTLYVFGTGTQLTVL |
| | | TTTPAPRPPTPAPTIASQPLSLRPEACRPAAGGAVHTRGLDF |
| | | ACDIYIWAPLAGTCGVLLLSLVITLYCKRGRKKLLYIFKQPF |
| | | MRPVQTTQEEDGCSCRFPEEEEGGCELRVKFSRSADAPAY |
| | | QQGQNQLYNELNLGRREEYDVLDKRRGRDPEMGGKPRRK |
| | | NPQEGLYNELQKDKMAEAYSEIGMKGERRRGKGHDGLYQ |
| | | GLSTATKDTYDALHMQALPPR |
| CG#c182 | 727 | atggccctccctgtcaccgccctgctgcttccgctggctcttctgctccacgccgctcggcccg |
| | | aagtgcagcttcaacaatcaggaccaggactcgtcaaaccatcacagaccctctccctcacat |
| | | gtgccatctccggggactccatgttgagcaattccgacacttggaattggattagacaaagccc |
| | | gtcccggggtctggaatggttgggacgcacctaccaccggtctacttggtacgacgactacg |
| | | cgtcatccgtgcggggaagagtgtccatcaacgtggacacctccaagaaccagtacagcctg |
| | | cagcttaatgccgtgactcctgaggatacgggcgtctactactgcgcccgcgtccgcctgcaa |
| | | gacgggaacagctggagcgatgcattcgatgtctggggccagggaactatggtcaccgtgtc |
| | | gtctgggggcggtggatcgggtggcgggggttcggggggcggcggctctcagtccgctctt |
| | | acccaaccggcctcagcctcggggagccccggccagagcgtgaccatttcctgcaccggc |
| | | acttcatccgacgtgggcggctacaactacgtgtcctggtaccaacagcacccgggaaaggc |
| | | ccccaagctcatgatctacgacgtgtccaacaggccctcgggagtgtccaaccggttctcgg |
| | | gttcgaaatcgggaaacacagccagcctgaccatcagcggactgcaggctgaagatgaagc |
| | | cgactactactgctcctcctacacctcgtcatccacgctctacgtgttcggcactggaactcagc |
| | | tgactgtgctgggagggggagggagtgaaattgtgatgacccagtcacccgccactcttagc |
| | | ctttcacccggtgagcgcgcaaccctgtcttgcagagcctcccaagacatctcaaaatacctta |
| | | attggtatcaacagaagcccggacaggctcctcgccttctgatctaccacaccagccggctcc |
| | | attctggaatccctgccaggttcagcggtagcggatctgggaccgactacaccctcactatca |
| | | gctcactgcagccagaggacttcgctgtctatttctgtcagcaagggaacaccctgccctaca |
| | | cctttggacagggcaccaagctcgagattaaaggtggaggtggcagcggaggaggtgggt |
| | | ccggcggtggaggaagccaggtccaactccaagaaagcggaccgggtcttgtgaagccat |
| | | cagaaactctttcactgacttgtactgtgagcggagtgtctctccccgattacggggtgtcttgg |
| | | atcagacagccaccggggaagggtctggaatggattggagtgatttggggctctgagactac |
| | | ttactaccaatcatccctcaagtcacgcgtcaccatctcaaaggacaactctaagaatcaggtg |
| | | tcactgaaactgtcatctgtgaccgcagccgacaccgccgtgtactattgcgctaagcattact |
| | | attatggcgggagctacgcaatggattactggggacagggtactctggtcaccgtgtccagca |
| | | ccactaccccagcaccgaggccacccaccccggctcctaccatcgcctcccagcctctgtcc |
| | | ctgcgtccggaggcatgtagacccgcagctggtggggccgtgcatacccggggtcttgactt |
| | | cgcctgcgatatctacatttgggcccctctggctggtacttgcggggtcctgctgctttcactcgt |
| | | gatcactctttactgtaagcgcggtcggaagaagctgctgtacatctttaagcaacccttcatga |
| | | ggcctgtgcagactactcaagaggaggacggctgttcatgccggttcccagaggaggagga |
| | | aggcggctgcgaactgcgcgtgaaattcagccgcagcgcagatgctccagcctaccagcag |
| | | gggcagaaccagctctacaacgaactcaatcttggtcggagagaggagtacgacgtgctgg |
| | | acaagcggagaggacgggacccagaaatgggcgggaagccgcgcagaaagaatcccca |
| | | agagggcctgtacaacgagctccaaaaggataagatggcagaagcctatagcgagattggt |
| | | atgaaaggggaacgcagaagaggcaaaggccacgacggactgtaccagggactcagcac |
| | | cgccaccaaggacacctatgacgctcttcacatgcaggccctgccgcctcgg |
| | 728 | MALPVTALLLPLALLLHAARPEVQLQQSGPGLVKPSQTLSL |
| | | TCAISGDSMLSNSDTWNWIRQSPSRGLEWLGRTYHRSTWY |
| | | DDYASSVRGRVSINVDTSKNQYSLQLNAVTPEDTGVYYCA |
| | | RVRLQDGNSWSDAFDVWGQGTMVTVSSGGGGSGGGGSG |
| | | GGGSQSALTQPASASGSPGQSVTISCTGTSSDVGGYNYVS |
| | | WYQQHPGKAPKLMIYDVSNRPSGVSNRFSGSKSGNTASLT |
| | | ISGLQAEDEADYYCSSYTSSSTLYVFGTGTQLTVLGGGGSE |
| | | IVMTQSPATLSLSPGERATLSCRASQDISKYLNWYQQKPGQ |
| | | APRLLIYHTSRLHSGIPARFSGSGSGTDYTLTISSLQPEDFAV |
| | | YFCQQGNTLPYTFGQGTKLEIKGGGGSGGGGSGGGGSQVQ |
| | | LQESGPGLVKPSETLSLTCTVSGVSLPDYGVSWIRQPPGKG |
| | | LEWIGVIWGSETTYYQSSLKSRVTISKDNSKNQVSLKLSSV |
| | | TAADTAVYYCAKHYYYGGSYAMDYWGQGTLVTVSSTTT |
| | | PAPRPPTPAPTIASQPLSLRPEACRPAAGGAVHTRGLDFAC |
| | | DIYIWAPLAGTCGVLLLSLVITLYCKRGRKKLLYIFKQPFM |
| | | RPVQTTQEEDGCSCRFPEEEEGGCELRVKFSRSADAPAYQQ |
| | | GQNQLYNELNLGRREEYDVLDKRRGRDPEMGGKPRRKNP |
| | | QEGLYNELQKDKMAEAYSEIGMKGERRRGKGHDGLYQGL |
| | | STATKDTYDALHMQALPPR |
| CG#c188 | 729 | atggccctccctgtcaccgccctgctgcttccgctggctcttctgctccacgccgctcggcccc |
| | | agtccgctcttacccaaccggcctcagcctcggggagccccggccagagcgtgaccatttcc |
| | | tgcaccggcacttcatccgacgtgggcggctacaactacgtgtcctggtaccaacagcaccc |
| | | gggaaaggcccccaagctcatgatctacgacgtgtccaacaggccctcgggagtgtccaac |
| | | cggttctcgggttcgaaatcgggaaacacagccagcctgaccatcagcggactgcaggctg |
| | | aagatgaagccgactactactgctcctcctacacctcgtcatccacgctctacgtgttcggcact |
| | | ggaactcagctgactgtgctgggcggaggaggctccgaagtgcagcttcaacaatcaggac |
| | | caggactcgtcaaaccatcacagaccctctccctcacatgtgccatctccggggactccatgtt |
| | | gagcaattccgacacttggaattggattagacaaagcccgtcccggggtctggaatggttggg |
| | | acgcacctaccaccggtctacttggtacgacgactacgcgtcatccgtgcggggaagagtgt |
| | | ccatcaacgtggacacctccaagaaccagtacagcctgcagcttaatgccgtgactcctgag |
| | | gatacgggcgtctactactgcgcccgcgtccgcctgcaagacgggaacagctggagcgatg |
| | | cattcgatgtctggggccagggaactatggtcaccgtgtcgtctggagggggagggagtgaa |
| | | attgtgatgacccagtcacccgccactcttagcctttcacccggtgagcgcgcaaccctgtctt |
| | | gcagagcctcccaagacatctcaaaataccttaattggtatcaacagaagcccggacaggctc |
| | | ctcgccttctgatctaccacaccagccggctccattctggaatccctgccaggttcagcggtag |
| | | cggatctgggaccgactacaccctcactatcagctcactgcagccagaggacttcgctgtcta |
| | | tttctgtcagcaagggaacaccctgccctacacctttggacagggcaccaagctcgagattaa |
| | | aggtggaggtggcagcggaggaggtgggtccggcggtggaggaagccaggtccaactcc |
| | | aagaaagcggaccgggtcttgtgaagccatcagaaactctttcactgacttgtactgtgagcg |
| | | gagtgtctctccccgattacggggtgtcttggatcagacagccaccggggaagggtctggaa |
| | | tggattggagtgatttggggctctgagactacttactaccaatcatccctcaagtcacgcgtcac |
| | | catctcaaaggacaactctaagaatcaggtgtcactgaaactgtcatctgtgaccgcagccga |
| | | caccgccgtgtactattgcgctaagcattactattatggcgggagctacgcaatggattactgg |
| | | ggacagggtactctggtcaccgtgtccagcaccactaccccagcaccgaggccacccaccc |
| | | cggctcctaccatcgcctcccagcctctgtccctgcgtccggaggcatgtagacccgcagct |
| | | ggtggggccgtgcatacccggggtcttgacttcgcctgcgatatctacatttgggcccctctgg |
| | | ctggtacttgcggggtcctgctgctttcactcgtgatcactctttactgtaagcgcggtcggaag |
| | | aagctgctgtacatctttaagcaacccttcatgaggcctgtgcagactactcaagaggaggac |
| | | ggctgttcatgccggttcccagaggaggaggaaggcggctgcgaactgcgcgtgaaattca |
| | | gccgcagcgcagatgctccagcctaccagcaggggcagaaccagctctacaacgaactca |
| | | atcttggtcggagagaggagtacgacgtgctggacaagcggagaggacgggacccagaaa |
| | | tgggcgggaagccgcgcagaaagaatccccaagagggcctgtacaacgagctccaaaag |
| | | gataagatggcagaagcctatagcgagattggtatgaaaggggaacgcagaagaggcaaa |
| | | ggccacgacggactgtaccagggactcagcaccgccaccaaggacacctatgacgctcttc |
| | | acatgcaggccctgccgcctcgg |
| | 730 | MALPVTALLLPLALLLHAARPQSALTQPASASGSPGQSVTI |
| | | SCTGTSSDVGGYNYVSWYQQHPGKAPKLMIYDVSNRPSG |
| | | VSNRFSGSKSGNTASLTISGLQAEDEADYYCSSYTSSSTLY |
| | | VFGTGTQLTVLGGGGSEVQLQQSGPGLVKPSQTLSLTCAIS |
| | | GDSMLSNSDTWNWIRQSPSRGLEWLGRTYHRSTWYDDYA |
| | | SSVRGRVSINVDTSKNQYSLQLNAVTPEDTGVYYCARVRL |
| | | QDGNSWSDAFDVWGQGTMVTVSSGGGGSEIVMTQSPATL |
| | | SLSPGERATLSCRASQDISKYLNWYQQKPGQAPRLLIYHTS |
| | | RLHSGIPARFSGSGSGTDYTLTISSLQPEDFAVYFCQQGNTL |
| | | PYTFGQGTKLEIKGGGGSGGGGSGGGGSQVQLQESGPGLV |
| | | KPSETLSLTCTVSGVSLPDYGVSWIRQPPGKGLEWIGVIWG |
| | | SETTYYQSSLKSRVTISKDNSKNQVSLKLSSVTAADTAVYY |
| | | CAKHYYYGGSYAMDYWGQGTLVTVSSTTTPAPRPPTPAPT |
| | | IASQPLSLRPEACRPAAGGAVHTRGLDFACDIYIWAPLAGT |
| | | CGVLLLSLVITLYCKRGRKKLLYIFKQPFMRPVQTTQEEDG |
| | | CSCRFPEEEEGGCELRVKFSRSADAPAYQQGQNQLYNELN |
| | | LGRREEYDVLDKRRGRDPEMGGKPRRKNPQEGLYNELQK |
| | | DKMAEAYSEIGMKGERRRGKGHDGLYQGLSTATKDTYDA |
| | | LHMQALPPR |
| CG#c224 | 731 | atggccctgcccgtgactgcgctcctgcttccgttggccctgctcctgcatgccgccagacctc |
| | | agtccgctctgactcagccggcctcagcttcggggtcccctggtcaaagcgtcactatttcctg |
| | | taccggaacctcatcagacgtgggcggctacaattacgtgtcctggtaccaacagcaccccg |
| | | gaaaggctcctaagcttatgatctacgacgtgtccaaccggccgtcaggagtgtccaacagat |
| | | tctccggctccaagagcggaaacactgccagcttgaccattagcggcttgcaggccgagga |
| | | cgaagccgactactactgctctagctacacatcctcgtctaccctctacgtgtttggaacgggg |
| | | acccagctgactgtgctcgggggtggaggatcagaggtgcaactccagcagtccggtcctg |
| | | gcctcgtgaaaccgtcccaaaccctgtccctgacttgcgccatctcgggcgactccatgctgt |
| | | ccaattccgacacctggaactggattagacaatcgcctagccggggactcgaatggctgggc |
| | | cggacctaccaccggtccacgtggtatgacgactacgcaagctccgtccggggaagggtgt |
| | | ccattaacgtcgatacctccaagaaccagtacagccttcagctgaacgctgtgacccccgag |
| | | gataccggcgtctactactgtgcaagagtgcgattgcaggatggaaactcgtggtcggacgc |
| | | attcgatgtctggggacagggaactatggtgaccgtgtcctcgggcggaggcgggagcgga |
| | | ggaggaggctctggcggaggaggaagcgagattgtcatgactcagtccccggccacactct |
| | | ccctgtcacccggagaaagagcaaccctgagctgcagggcgtcccaggacatctcgaagta |
| | | cctgaactggtaccagcagaagcctggacaagcaccccgcctcctgatctaccacacctcgc |
| | | ggctgcattcgggaatccccgccagattctcagggagcggatcaggaaccgactacaccct |
| | | gactatctcgagcctgcaaccagaggatttcgccgtgtacttctgccagcaaggaaacaccct |
| | | gccctacacctttggacagggaaccaagctcgagattaaggggggtggtggatcgggaggg |
| | | ggtggatcaggaggaggcggctcacaagtccagctgcaagaatccggtccgggacttgtga |
| | | agccgtccgaaaccctgtcactgacttgcactgtgtccggggtgtcattgcccgactacggcg |
| | | tgagctggattcggcagccccctggaaagggattggaatggatcggcgtgatctggggttcg |
| | | gaaactacctactatcagtcctcactgaagtcccgcgtgaccatcagcaaggataattccaaaa |
| | | accaagtgtctctgaagctctccagcgtcactgccgccgatactgccgtgtactactgcgcca |
| | | agcactactattacggcggttcgtacgccatggactactggggccaagggacactcgtgacc |
| | | gtgtcatccaccactaccccagcaccgaggccacccaccccggctcctaccatcgcctccca |
| | | gcctctgtccctgcgtccggaggcatgtagacccgcagctggtggggccgtgcatacccgg |
| | | ggtcttgacttcgcctgcgatatctacatttgggcccctctggctggtacttgcggggtcctgct |
| | | gctttcactcgtgatcactctttactgtaagcgcggtcggaagaagctgctgtacatctttaagca |
| | | acccttcatgaggcctgtgcagactactcaagaggaggacggctgttcatgccggttcccaga |
| | | ggaggaggaaggcggctgcgaactgcgcgtgaaattcagccgcagcgcagatgctccagc |
| | | ctaccagcaggggcagaaccagctctacaacgaactcaatcttggtcggagagaggagtac |
| | | gacgtgctggacaagcggagaggacgggacccagaaatgggcgggaagccgcgcagaa |
| | | agaatccccaagagggcctgtacaacgagctccaaaaggataagatggcagaagcctatag |
| | | cgagattggtatgaaaggggaacgcagaagaggcaaaggccacgacggactgtaccagg |
| | | gactcagcaccgccaccaaggacacctatgacgctcttcacatgcaggccctgccgcctcgg |
| | 732 | MALPVTALLLPLALLLHAARPQSALTQPASASGSPGQSVTI |
| | | SCTGTSSDVGGYNYVSWYQQHPGKAPKLMIYDVSNRPSG |
| | | VSNRFSGSKSGNTASLTISGLQAEDEADYYCSSYTSSSTLY |
| | | VFGTGTQLTVLGGGGSEVQLQQSGPGLVKPSQTLSLTCAIS |
| | | GDSMLSNSDTWNWIRQSPSRGLEWLGRTYHRSTWYDDYA |
| | | SSVRGRVSINVDTSKNQYSLQLNAVTPEDIGVYYCARVRL |
| | | QDGNSWSDAFDVWGQGTMVTVSSGGGGSGGGGSGGGGS |
| | | EIVMTQSPATLSLSPGERATLSCRASQDISKYLNWYQQKPG |
| | | QAPRLLIYHTSRLHSGIPARFSGSGSGTDYTLTISSLQPEDFA |
| | | VYFCQQGNTLPYTFGQGTKLEIKGGGGSGGGGSGGGGSQV |
| | | QLQESGPGLVKPSETLSLTCTVSGVSLPDYGVSWIRQPPGK |
| | | GLEWIGVIWGSETTYYQSSLKSRVTISKDNSKNQVSLKLSS |
| | | VTAADTAVYYCAKHYYYGGSYAMDYWGQGTLVTVSSTT |
| | | TPAPRPPTPAPTIASQPLSLRPEACRPAAGGAVHTRGLDFAC |
| | | DIYIWAPLAGTCGVLLLSLVITLYCKRGRKKLLYIFKQPFM |
| | | RPVQTTQEEDGCSCRFPEEEEGGCELRVKFSRSADAPAYQQ |
| | | GQNQLYNELNLGRREEYDVLDKRRGRDPEMGGKPRRKNP |
| | | QEGLYNELQKDKMAEAYSEIGMKGERRRGKGHDGLYQGL |
| | | STATKDTYDALHMQALPPR |
| CG#c227 | 733 | atggccctgcccgtgactgcgctcctgcttccgttggccctgctcctgcatgccgccagacctc |
| | | agtccgctctgactcagccggcctcagcttcggggtcccctggtcaaagcgtcactatttcctg |
| | | taccggaacctcatcagacgtgggcggctacaattacgtgtcctggtaccaacagcaccccg |
| | | gaaaggctcctaagcttatgatctacgacgtgtccaaccggccgtcaggagtgtccaacagat |
| | | tctccggctccaagagcggaaacactgccagcttgaccattagcggcttgcaggccgagga |
| | | cgaagccgactactactgctctagctacacatcctcgtctaccctctacgtgtttggaacgggg |
| | | acccagctgactgtgctcgggggtggaggatcagaggtgcaactccagcagtccggtcctg |
| | | gcctcgtgaaaccgtcccaaaccctgtccctgacttgcgccatctcgggcgactccatgctgt |
| | | ccaattccgacacctggaactggattagacaatcgcctagccggggactcgaatggctgggc |
| | | cggacctaccaccggtccacgtggtatgacgactacgcaagctccgtccggggaagggtgt |
| | | ccattaacgtcgatacctccaagaaccagtacagccttcagctgaacgctgtgacccccgag |
| | | gataccggcgtctactactgtgcaagagtgcgattgcaggatggaaactcgtggtcggacgc |
| | | attcgatgtctggggacagggaactatggtcactgtgtcctccggcggtggaggctcggggg |
| | | ggggcggctcaggaggaggcggctcacaagtccagctgcaagaatccggtccgggacttg |
| | | tgaagccgtccgaaaccctgtcactgacttgcactgtgtccggggtgtcattgcccgactacg |
| | | gcgtgagctggattcggcagccccctggaaagggattggaatggatcggcgtgatctggggt |
| | | tcggaaactacctactatcagtcctcactgaagtcccgcgtgaccatcagcaaggataattcca |
| | | aaaaccaagtgtctctgaagctctccagcgtcactgccgccgatactgccgtgtactactgcg |
| | | ccaagcactactattacggcggttcgtacgccatggactactggggacaaggcactcttgtga |
| | | ctgtgtcaagcggcggtggagggagcggtgggggcggttcaggaggaggcggatcagag |
| | | atcgtgatgacccaatccccagccaccctgtccctcagccctggagaaagagccaccctgag |
| | | ctgccgggcctcccaggatatcagcaagtacttgaactggtaccaacaaaagccggggcag |
| | | gcgccccggctcctgatctaccacacctcgcgcctccactcaggtatccccgccagattctca |
| | | gggagcggctccggtactgactacaccctgactatttcctcactgcagccagaggactttgcc |
| | | gtgtacttctgccagcagggaaacactctgccgtacaccttcgggcagggaacgaagcttga |
| | | aattaagaccactaccccagcaccgaggccacccaccccggctcctaccatcgcctcccag |
| | | cctctgtccctgcgtccggaggcatgtagacccgcagctggtggggccgtgcatacccggg |
| | | gtcttgacttcgcctgcgatatctacatttgggcccctctggctggtacttgcggggtcctgctg |
| | | ctttcactcgtgatcactctttactgtaagcgcggtcggaagaagctgctgtacatctttaagcaa |
| | | cccttcatgaggcctgtgcagactactcaagaggaggacggctgttcatgccggttcccaga |
| | | ggaggaggaaggggctgcgaactgcgcgtgaaattcagccgcagcgcagatgctccagc |
| | | ctaccagcaggggcagaaccagctctacaacgaactcaatcttggtcggagagaggagtac |
| | | gacgtgctggacaagcggagaggacgggacccagaaatgggcgggaagccgcgcagaa |
| | | agaatccccaagagggcctgtacaacgagctccaaaaggataagatggcagaagcctatag |
| | | cgagattggtatgaaaggggaacgcagaagaggcaaaggccacgacggactgtaccagg |
| | | gactcagcaccgccaccaaggacacctatgacgctcttcacatgcaggccctgccgcctcgg |
| | 734 | MALPVTALLLPLALLLHAARPQSALTQPASASGSPGQSVTI |
| | | SCTGTSSDVGGYNYVSWYQQHPGKAPKLMIYDVSNRPSG |
| | | VSNRFSGSKSGNTASLTISGLQAEDEADYYCSSYTSSSTLY |
| | | VFGTGTQLTVLGGGGSEVQLQQSGPGLVKPSQTLSLTCAIS |
| | | GDSMLSNSDTWNWIRQSPSRGLEWLGRTYHRSTWYDDYA |
| | | SSVRGRVSINVDTSKNQYSLQLNAVTPEDIGVYYCARVRL |
| | | QDGNSWSDAFDVWGQGTMVTVSSGGGGSGGGGSGGGGS |
| | | QVQLQESGPGLVKPSETLSLTCTVSGVSLPDYGVSWIRQPP |
| | | GKGLEWIGVIWGSETTYYQSSLKSRVTISKDNSKNQVSLKL |
| | | SSVTAADTAVYYCAKHYYYGGSYAMDYWGQGTLVTVSS |
| | | GGGGSGGGGSGGGGSEIVMTQSPATLSLSPGERATLSCRAS |
| | | QDISKYLNWYQQKPGQAPRLLIYHTSRLHSGIPARFSGSGS |
| | | GTDYTLTISSLQPEDFAVYFCQQGNTLPYTFGQGTKLEIKTT |
| | | TPAPRPPTPAPTIASQPLSLRPEACRPAAGGAVHTRGLDFAC |
| | | DIYIWAPLAGTCGVLLLSLVITLYCKRGRKKLLYIFKQPFM |
| | | RPVQTTQEEDGCSCRFPEEEEGGCELRVKFSRSADAPAYQQ |
| | | GQNQLYNELNLGRREEYDVLDKRRGRDPEMGGKPRRKNP |
| | | QEGLYNELQKDKMAEAYSEIGMKGERRRGKGHDGLYQGL |
| | | STATKDTYDALHMQALPPR |

Dual CD19-CD22 CARs
CG#c201

| | | |
|---|---|---|
| Full length | 735 | atggccctccctgtcaccgccctgctgcttccgctggctcttctgctccacgccgctcggcccg |
| CD19-CD22 | | aagtgcagctgcagcagtcagggcctggcctggtcaagccgtcgcagaccctctccctgac |
| Dual CAR | | atgcgccattagcggggactccatgctgagcaactcggacacctggaactggattcggcagt |
| nucleic acid | | ccccttcccggggactcgagtggctcggacgcacctaccatcggagcacttggtacgacga |
| | | ctacgcctcctccgtgagaggtcgcgtgtcgatcaacgtggatacctcgaagaaccagtatag |
| | | cttgcaactgaacgccgtgacccctgaggataccggagtgtactattgtgcgagagtcaggct |
| | | gcaagacggaaactcctggtccgacgcatttgatgtctggggacagggtactatggtcacggt |
| | | gtcatctggaggcggaggatcgcaaagcgccctgactcagccggcttcggctagcggttca |
| | | ccggggcagtccgtgactatctcctgcaccgggacttcctccgacgtgggaggctacaattac |
| | | gtgtcctggtaccagcaacaccccggcaaagccccaaagctgatgatctacgacgtcagcaa |
| | | cagacccagcggagtgtccaaccggttcagcggctccaagtccggcaacaccgcctccctg |
| | | accatcagcgggcttcaggccgaagatgaggcggattactactgctcctcgtacacctcaag |
| | | ctcaactctgtacgtgttcggcaccggtactcagctcaccgtgctgaccactaccccagcacc |
| | | gaggccacccaccccggctcctaccatcgcctcccagcctctgtccctgcgtccggaggcat |
| | | gtagacccgcagctggtggggccgtgcatacccggggtcttgacttcgcctgcgatatctaca |
| | | tttgggcccctctggctggtacttgcggggtcctgctgctttcactcgtgatcactctttactgtaa |
| | | gcgcggtcggaagaagctgctgtacatctttaagcaacccttcatgaggcctgtgcagactac |
| | | tcaagaggaggacggctgttcatgccggttcccagaggaggaggaaggggctgcgaact |
| | | gcgcgtgaaattcagccgcagcgcagatgctccagcctaccagcaggggcagaaccagct |
| | | ctacaacgaactcaatcttggtcggagagaggagtacgacgtgctggacaagcggagagga |
| | | cgggacccagaaatgggcgggaagccgcgcagaaagaatccccaagagggcctgtacaa |
| | | cgagctccaaaaggataagatggcagaagcctatagcgagattggtatgaaaggggaacgc |
| | | agaagaggcaaaggccacgacggactgtaccagggactcagcaccgccaccaaggacac |
| | | ctatgacgctcttcacatgcaggccctgccgcctcggggaagcggagctactaacttcagcct |
| | | gctgaagcaggctggagacgtggaggagaaccctggacctatggccttaccagtgaccgcc |
| | | ttgctcctgccgctggccttgctgctccacgccgccaggccggaaattgtgatgacccagtca |
| | | cccgccactcttagcctttcacccggtgagcgcgcaaccctgtcttgcagagcctcccaagac |
| | | atctcaaaataccttaattggtatcaacagaagcccggacaggctcctcgccttctgattacca |
| | | caccagccggctccattctggaatccctgccaggttcagcggtagcggatctgggaccgact |
| | | acaccctcactatcagctcactgcagccagaggacttcgctgtctatttctgtcagcaagggaa |
| | | caccctgccctacacctttggacagggcaccaagctcgagattaaaggtggaggtggcagc |
| | | ggaggaggtgggtccggcggtggaggaagccaggtccaactccaagaaagcggaccgg |
| | | gtcttgtgaagccatcagaaactctttcactgacttgtactgtgagcggagtgtctctccccgatt |
| | | acggggtgtcttggatcagacagccaccggggaagggtctggaatggattggagtgatttgg |
| | | ggctctgagactacttactaccaatcatccctcaagtcacgcgtcaccatctcaaaggacaact |
| | | ctaagaatcaggtgtcactgaaactgtcatctgtgaccgcagccgacaccgccgtgtactattg |
| | | cgctaagcattactattatggcgggagctacgcaatggattactggggacagggtactctggt |
| | | caccgtgtccagcaccacgacgccagcgccgcgaccaccaacaccggcgcccaccatcg |
| | | cgtcgcagcccctgtccctgcgcccagaggcgtgccggccagcggcggggggcgcagtg |
| | | cacacgagggggctggacttcgcctgtgatatctacatctgggcgcccttggccgggacttgt |
| | | ggggtccttctcctgtcactggttatcaccctttactgcaaacggggcagaaagaaactcctgt |
| | | atatattcaaacaaccatttatgagaccagtacaaactactcaagaggaagatggctgtagctg |
| | | ccgatttccagaagaagaagaaggaggatgtgaactgagagtgaagttcagcaggagcgca |
| | | gacgcccccgcgtaccagcagggccagaaccagctctataacgagctcaatctaggacgaa |
| | | gagaggagtacgatgttttggacaagagacgtggccgggaccctgagatggggggaaagc |
| | | cgagaaggaagaaccctcaggaaggcctgtacaatgaactgcagaaagataagatggcgg |
| | | aggcctacagtgagattgggatgaaaggcgagcgccggaggggcaaggggcacgatggc |
| | | ctttaccagggtctcagtacagccaccaaggacacctacgacgcccttcacatgcaggccct |
| | | gccccctcgc |
| Full length | 736 | MALPVTALLLPLALLLHAARPEVQLQQSGPGLVKPSQTLSL |
| CD19-CD22 | | TCAISGDSMLSNSDTWNWIRQSPSRGLEWLGRTYHRSTWY |
| Dual CAR | | DDYASSVRGRVSINVDTSKNQYSLQLNAVTPEDTGVYYCA |
| amino acid | | RVRLQDGNSWSDAFDVWGQGTMVTVSSGGGGSQSALTQP |
| | | ASASGSPGQSVTISCTGTSSDVGGYNYVSWYQQHPGKAPK |
| | | LMIYDVSNRPSGVSNRFSGSKSGNTASLTISGLQAEDEADY |
| | | YCSSYTSSSTLYVFGTGTQLTVLTTTPAPRPPTPAPTIASQPL |
| | | SLRPEACRPAAGGAVHTRGLDFACDIYIWAPLAGTCGVLL |
| | | LSLVITLYCKRGRKKLLYIFKQPFMRPVQTTQEEDGCSCRF |
| | | PEEEEGGCELRVKFSRSADAPAYQQGQNQLYNELNLGRRE |
| | | EYDVLDKRRGRDPEMGGKPRRKNPQEGLYNELQKDKMA |
| | | EAYSEIGMKGERRRGKGHDGLYQGLSTATKDTYDALHMQ |
| | | ALPPRGSGATNFSLLKQAGDVEENPGPMALPVTALLLPLAL |
| | | LLHAARPEIVMTQSPATLSLSPGERATLSCRASQDISKYLN |
| | | WYQQKPGQAPRLLIYHTSRLHSGIPARFSGSGSGTDYTLTIS |
| | | SLQPEDFAVYFCQQGNTLPYTFGQGTKLEIKGGGGSGGGG |
| | | SGGGGSQVQLQESGPGLVKPSETLSLTCTVSGVSLPDYGVS |
| | | WIRQPPGKGLEWIGVIWGSETTYYQSSLKSRVTISKDNSKN |
| | | QVSLKLSSVTAADTAVYYCAKHYYYGGSYAMDYWGQGT |
| | | LVTVSSTTTPAPRPPTPAPTIASQPLSLRPEACRPAAGGAVH |
| | | TRGLDFACDIYIWAPLAGTCGVLLLSLVITLYCKRGRKKLL |
| | | YIFKQPFMRPVQTTQEEDGCSCRFPEEEEGGCELRVKFSRS |
| | | ADAPAYQQGQNQLYNELNLGRREEYDVLDKRRGRDPEM |
| | | GGKPRRKNPQEGLYNELQKDKMAEAYSEIGMKGERRRGK |
| | | GHDGLYQGLSTATKDTYDALHMQALPPR |
| CD22 CAR | 737 | MALPVTALLLPLALLLHAARPEVQLQQSGPGLVKPSQTLSL |
| (with P2A site) | | TCAISGDSMLSNSDTWNWIRQSPSRGLEWLGRTYHRSTWY |
| | | DDYASSVRGRVSINVDTSKNQYSLQLNAVTPEDIGVYYCA |
| | | RVRLQDGNSWSDAFDVWGQGTMVTVSSGGGGSQSALTQP |
| | | ASASGSPGQSVTISCTGTSSDVGGYNYVSWYQQHPGKAPK |
| | | LMIYDVSNRPSGVSNRFSGSKSGNTASLTISGLQAEDEADY |
| | | YCSSYTSSSTLYVFGTGTQLTVLTTTPAPRPPTPAPTIASQPL |
| | | SLRPEACRPAAGGAVHTRGLDFACDIYIWAPLAGTCGVLL |
| | | LSLVITLYCKRGRKKLLYIFKQPFMRPVQTTQEEDGCSCRF |
| | | PEEEEGGCELRVKFSRSADAPAYQQGQNQLYNELNLGRRE |
| | | EYDVLDKRRGRDPEMGGKPRRKNPQEGLYNELQKDKMA |
| | | EAYSEIGMKGERRRGKGHDGLYQGLSTATKDTYDALHMQ |
| | | ALPPRGSGATNFSLLKQAGDVEENPG |
| CD19 CAR | 738 | PMALPVTALLLPLALLLHAARPEIVMTQSPATLSLSPGERA |
| | | TLSCRASQDISKYLNWYQQKPGQAPRLLIYHTSRLHSGIPA |
| | | RFSGSGSGTDYTLTISSLQPEDFAVYFCQQGNTLPYTFGQG |
| | | TKLEIKGGGGSGGGGSGGGGSQVQLQESGPGLVKPSETLSL |
| | | TCTVSGVSLPDYGVSWIRQPPGKGLEWIGVIWGSETTYYQS |
| | | SLKSRVTISKDNSKNQVSLKLSSVTAADTAVYYCAKHYYY |
| | | GGSYAMDYWGQGTLVTVSSTTTPAPRPPTPAPTIASQPLSL |
| | | RPEACRPAAGGAVHTRGLDFACDIYIWAPLAGTCGVLLLS |
| | | LVITLYCKRGRKKLLYIFKQPFMRPVQTTQEEDGCSCRFPE |
| | | EEEGGCELRVKFSRSADAPAYQQGQNQLYNELNLGRREEY |
| | | DVLDKRRGRDPEMGGKPRRKNPQEGLYNELQKDKMAEA |
| | | YSEIGMKGERRRGKGHDGLYQGLSTATKDTYDALHMQAL |
| | | PPR |

CG#c203

| | | |
|---|---|---|
| Full length | 739 | atggccttaccagtgaccgccttgctcctgccgctggccttgctgctccacgccgccaggccg |
| CD19-CD22 | | gaaattgtgatgacccagtcacccgccactcttagcctttcacccggtgagcgcgcaaccctg |
| Dual CAR | | tcttgcagagcctcccaagacatctcaaaataccttaattggtatcaacagaagcccggacag |
| nucleic acid | | gctcctcgccttctgatctaccacaccagccggctccattctggaatccctgccaggttcagcg |
| | | gtagcggatctgggaccgactacaccctcactatcagctcactgcagccagaggacttcgct |
| | | gtctatttctgtcagcaagggaacaccctgccctacacctttggacagggcaccaagctcgag |
| | | attaaaggtggaggtggcagcggaggaggtgggtccggcggtggaggaagccaggtcca |
| | | actccaagaaagcggaccgggtcttgtgaagccatcagaaactctttcactgacttgtactgtg |
| | | agcggagtgtctctccccgattacggggtgtcttggatcagacagccaccggggaagggtct |
| | | ggaatggattggagtgatttggggctctgagactacttactaccaatcatccctcaagtcacgc |
| | | gtcaccatctcaaaggacaactctaagaatcaggtgtcactgaaactgtcatctgtgaccgcag |
| | | ccgacaccgccgtgtactattgcgctaagcattactattatggcgggagctacgcaatggatta |
| | | ctggggacagggtactctggtcaccgtgtccagcaccacgacgccagcgccgcgaccacc |
| | | aacaccggcgcccaccatcgcgtcgcagcccctgtccctgcgcccagaggcgtgccggcc |
| | | agcggggggggcgcagtgcacacgagggggctggacttcgcctgtgatatctacatctgg |
| | | gcgcccttggccgggacttgtggggtccttctcctgtcactggttatcaccctttactgcaaacg |
| | | gggcagaaagaaactcctgtatatattcaaacaaccatttatgagaccagtacaaactactcaa |
| | | gaggaagatggctgtagctgccgatttccagaagaagaagaaggaggatgtgaactgagag |
| | | tgaagttcagcaggagcgcagacgcccccgcgtaccagcagggccagaaccagctctata |
| | | acgagctcaatctaggacgaagagaggagtacgatgttttggacaagagacgtggccggga |
| | | ccctgagatggggggaaagccgagaaggaagaaccctcaggaaggcctgtacaatgaact |
| | | gcagaaagataagatggcggaggcctacagtgagattgggatgaaaggcgagcgccgga |
| | | ggggcaaggggcacgatggcctttaccagggtctcagtacagccaccaaggacacctacga |
| | | cgcccttcacatgcaggccctgccccctcgcggaagcggagctactaacttcagcctgctga |
| | | agcaggctggagacgtggaggagaaccctggacctatggccctccctgtcaccgccctgct |
| | | gcttccgctggctcttctgctccacgccgctcggcccgaagtgcagctgcagcagtcagggc |
| | | ctggcctggtcaagccgtcgcagaccctctccctgacatgcgccattagcggggactccatg |
| | | ctgagcaactcggacacctggaactggattcggcagtccccttcccggggactcgagtggct |
| | | cggacgcacctaccatcggagcacttggtacgacgactacgcctcctccgtgagaggtcgc |
| | | gtgtcgatcaacgtggatacctcgaagaaccagtatagcttgcaactgaacgccgtgacccct |
| | | gaggataccggagtgtactattgtgcgagagtcaggctgcaagacggaaactcctggtccga |
| | | cgcatttgatgtctggggacagggtactatggtcacggtgtcatctggaggcggaggatcgca |
| | | aagcgccctgactcagccggcttcggctagcggttcaccggggcagtccgtgactatctcct |
| | | gcaccgggacttcctccgacgtgggaggctacaattacgtgtcctggtaccagcaacacccc |
| | | ggcaaagccccaaagctgatgatctacgacgtcagcaacagacccagcggagtgtccaacc |
| | | ggttcagcggctccaagtccggcaacaccgcctccctgaccatcagcgggcttcaggccga |
| | | agatgaggcggattactactgctcctcgtacacctcaagctcaactctgtacgtgttcggcacc |
| | | ggtactcagctcaccgtgctgaccactaccccagcaccgaggccacccaccccggctccta |
| | | ccatcgcctcccagcctctgtccctgcgtccggaggcatgtagacccgcagctggtggggcc |
| | | gtgcatacccggggtcttgacttcgcctgcgatatctacatttgggcccctctggctggtacttg |
| | | cggggtcctgctgctttcactcgtgatcactctttactgtaagcgcggtcggaagaagctgctgt |
| | | acatctttaagcaacccttcatgaggcctgtgcagactactcaagaggaggacggctgttcatg |
| | | ccggttcccagaggaggaggaaggcggctgcgaactgcgcgtgaaattcagccgcagcgc |
| | | agatgctccagcctaccagcaggggcagaaccagctctacaacgaactcaatcttggtcgga |
| | | gagaggagtacgacgtgctggacaagcggagaggacgggacccagaaatgggcgggaa |
| | | gccgcgcagaaagaatccccaagagggcctgtacaacgagctccaaaaggataagatggc |
| | | agaagcctatagcgagattggtatgaaaggggaacgcagaagaggcaaaggccacgacg |
| | | gactgtaccagggactcagcaccgccaccaaggacacctatgacgctcttcacatgcaggcc |
| | | ctgccgcctcgg |
| Full length | 740 | MALPVTALLLPLALLLHAARPEIVMTQSPATLSLSPGERAT |
| CD19-CD22 | | LSCRASQDISKYLNWYQQKPGQAPRLLIYHTSRLHSGIPAR |
| Dual CAR | | FSGSGSGTDYTLTISSLQPEDFAVYFCQQGNTLPYTFGQGT |
| amino acid | | KLEIKGGGGSGGGGSGGGGSQVQLQESGPGLVKPSETLSLT |
| | | CTVSGVSLPDYGVSWIRQPPGKGLEWIGVIWGSETTYYQSS |
| | | LKSRVTISKDNSKNQVSLKLSSVTAADTAVYYCAKHYYYG |
| | | GSYAMDYWGQGTLVTVSSTTTPAPRPPTPAPTIASQPLSLR |
| | | PEACRPAAGGAVHTRGLDFACDIYIWAPLAGTCGVLLLSL |
| | | VITLYCKRGRKKLLYIFKQPFMRPVQTTQEEDGCSCRFPEE |
| | | EEGGCELRVKFSRSADAPAYQQGQNQLYNELNLGRREEY |
| | | DVLDKRRGRDPEMGGKPRRKNPQEGLYNELQKDKMAEA |
| | | YSEIGMKGERRRGKGHDGLYQGLSTATKDTYDALHMQAL |
| | | PPRGSGATNFSLLKQAGDVEENPGPMALPVTALLLPLALLL |
| | | HAARPEVQLQQSGPGLVKPSQTLSLTCAISGDSMLSNSDT |
| | | WNWIRQSPSRGLEWLGRTYHRSTWYDDYASSVRGRVSIN |
| | | VDTSKNQYSLQLNAVTPEDTGVYYCARVRLQDGNSWSDA |
| | | FDVWGQGTMVTVSSGGGGSQSALTQPASASGSPGQSVTIS |
| | | CTGTSSDVGGYNYVSWYQQHPGKAPKLMIYDVSNRPSGV |
| | | SNRFSGSKSGNTASLTISGLQAEDEADYYCSSYTSSSTLYVF |
| | | GTGTQLTVLTTTPAPRPPTPAPTIASQPLSLRPEACRPAAGG |
| | | AVHTRGLDFACDIYIWAPLAGTCGVLLLSLVITLYCKRGRK |
| | | KLLYIFKQPFMRPVQTTQEEDGCSCRFPEEEEGGCELRVKF |
| | | SRSADAPAYQQGQNQLYNELNLGRREEYDVLDKRRGRDP |
| | | EMGGKPRRKNPQEGLYNELQKDKMAEAYSEIGMKGERRR |
| | | GKGHDGLYQGLSTATKDTYDALHMQALPPR |
| CD19 CAR | 741 | MALPVTALLLPLALLLHAARPEIVMTQSPATLSLSPGERAT |
| (with P2A site) | | LSCRASQDISKYLNWYQQKPGQAPRLLIYHTSRLHSGIPAR |
| | | FSGSGSGTDYTLTISSLQPEDFAVYFCQQGNTLPYTFGQGT |
| | | KLEIKGGGGSGGGGSGGGGSQVQLQESGPGLVKPSETLSLT |
| | | CTVSGVSLPDYGVSWIRQPPGKGLEWIGVIWGSETTYYQSS |
| | | LKSRVTISKDNSKNQVSLKLSSVTAADTAVYYCAKHYYYG |
| | | GSYAMDYWGQGTLVTVSSTTTPAPRPPTPAPTIASQPLSLR |
| | | PEACRPAAGGAVHTRGLDFACDIYIWAPLAGTCGVLLLSL |
| | | VITLYCKRGRKKLLYIFKQPFMRPVQTTQEEDGCSCRFPEE |
| | | EEGGCELRVKFSRSADAPAYQQGQNQLYNELNLGRREEY |
| | | DVLDKRRGRDPEMGGKPRRKNPQEGLYNELQKDKMAEA |
| | | YSEIGMKGERRRGKGHDGLYQGLSTATKDTYDALHMQAL |
| | | PPRGSGATNFSLLKQAGDVEENPG |
| CD22 CAR | 742 | PMALPVTALLLPLALLLHAARPEVQLQQSGPGLVKPSQTLS |
| | | LTCAISGDSMLSNSDTWNWIRQSPSRGLEWLGRTYHRSTW |
| | | YDDYASSVRGRVSINVDTSKNQYSLQLNAVTPEDTGVYYC |
| | | ARVRLQDGNSWSDAFDVWGQGTMVTVSSGGGGSQSALT |
| | | QPASASGSPGQSVTISCTGTSSDVGGYNYVSWYQQHPGKA |
| | | PKLMIYDVSNRPSGVSNRFSGSKSGNTASLTISGLQAEDEA |
| | | DYYCSSYTSSSTLYVFGTGTQLTVLTTTPAPRPPTPAPTIAS |
| | | QPLSLRPEACRPAAGGAVHTRGLDFACDIYIWAPLAGTCG |
| | | VLLLSLVITLYCKRGRKKLLYIFKQPFMRPVQTTQEEDGCS |
| | | CRFPEEEEGGCELRVKFSRSADAPAYQQGQNQLYNELNLG |
| | | RREEYDVLDKRRGRDPEMGGKPRRKNPQEGLYNELQKDK |
| | | MAEAYSEIGMKGERRRGKGHDGLYQGLSTATKDTYDALH |
| | | MQALPPR |

CG#c230

| | | |
|---|---|---|
| Full length | 743 | atggcacttcccgtcaccgccctgctgctcccactcgccctccttctgcacgccgcccgcccc |
| CD19-CD22 | | gaagtgcagctgcagcagtcaggaccgggcctggtcaaaccttcgcagactctgtccctgac |
| Dual CAR | | ttgcgctataagcggggactccatgctgagcaattcggacacttggaactggattcgccaaag |
| nucleic acid | | ccccagccggggtctggaatggctgggaaggacctaccatcgctctacttggtacgacgact |
| | | acgccagctccgtgcgaggacgcgtgtccatcaacgtggacacctccaagaaccagtactc |
| | | gcttcaactcaacgcagtgacccctgaagataccggagtctactattgcgcccgcgtgcggct |
| | | ccaggacgggaactcctggtcggacgctttcgatgtctggggacagggcactatggtcaccg |
| | | tcagctccggcggcggcggtagccaatcggcgctgacacagccggcttccgcctcgggatc |
| | | gcctggacagtcggtgaccatctcgtgcactggaacctcctccgacgtgggcggctacaatta |
| | | tgtgtcatggtaccagcagcacccgggaaaggcccctaagctgatgatctacgacgtgtcca |
| | | atagacctagcggggtgtcaaacagattctccggatccaaatccggaaacactgcctccctga |
| | | ccatttccggactgcaggccgaggacgaagccgattactactgctcctcttacacctcctcatc |
| | | caccctctacgtgtttgggactgggacccagctgaccgtcctcactaccaccccggccccgc |
| | | ggccccctacaccggcaccgactattgccagccagcctctctcgctgcggccggaggcctg |
| | | ccgcccagccgccggcggagccgtgcacacccgcggtctggacttcgcgtgcgatatctac |
| | | atctgggctccgctggccgggacttgtggcgtgctgctgctgtctctggtcatcacactgtact |
| | | gcaagcgcggaagaaagaagctgctctacatcttcaagcaacccttcatgcggcctgtgcag |
| | | accacccaggaagaggatggctgctcctgccggttcccggaggaagaagagggcggatgc |
| | | gaactgcgcgtgaagttcagccgaagcgccgacgccccggcctaccagcagggccagaa |
| | | ccaactgtacaacgaactcaacctgggtcggagagaagagtacgacgtgctggacaaaaga |
| | | cgcggcagggaccccgagatgggggaaagcctcgccgcaagaacccgcaggagggcc |
| | | tctacaacgagctgcagaaggacaagatggccgaagcctactcagagatcggcatgaaggg |
| | | ggagcggaggcgcgggaagggccacgacggtttgtaccaaggactttccactgcgaccaa |
| | | ggacacctacgatgccctccatatgcaagccctgccgccccggggttccggagctaccaact |
| | | tctcgctgttgaagcaggccggagatgtcgaggaaaacccgggacctatggccctgccagt |
| | | gaccgcgctcctgctgcccctggctctgctgcttcacgcggcccggcctgagattgtgatgac |
| | | tcagagcccggcgaccctgtccctgtcccccggggagagagcaaccctgtcgtgccgggc |
| | | ctcccaagacatctcaaagtacctcaattggtatcagcagaagccaggacaggctccacggtt |
| | | gctgatctaccacacttcgagactgcactcaggaatccccgcgcggttttccggttccggctcc |
| | | gggaccgactacaccctgaccatcagctcgctccagcctgaggatttcgcagtgtacttctgtc |
| | | agcaaggaaacacccttccatacaccttcggacagggtaccaagctggaaatcaagggagg |
| | | aggaggatctgggggcggtggttccggaggcggtggaagccaagtgcagctccaggaaa |
| | | gcggacccgggctggtcaagccgagcgaaaccctctcactgacttgtactgtgtccggagtg |
| | | tccctgcctgactatggagtgtcctggatccgacagccccccggaaagggtctggagtggatt |
| | | ggggtcatctggggctccgaaactacctactaccagagcagcctcaagagccgggtcaccat |
| | | ttcaaaggataactccaagaatcaagtgtccctgaagctgtcctcagtgacagccgcagacac |
| | | cgccgtgtactactgcgccaagcactactactacggaggctcctacgcaatggactactggg |
| | | gacaaggcactttggtcactgtgtcaagcaccaccacccctgcgcctcggcctcctaccccg |
| | | gctcccactatcgcgagccagccgctgagcctgcggcctgaggcttgccgaccggccgctg |
| | | gcggcgccgtgcatactcggggcctcgactttgcctgtgacatctacatctgggcccccctgg |
| | | ccggaacgtgcggagtgctgctgctgtcgctggtcattaccctgtattgcaaacgcggaagga |
| | | agaagctgttgtacattttcaagcagcccttcatgcgcccggtgcaaactactcaggaggaag |
| | | atggctgttcctgtcggttccccgaagaggaagaaggcggctgcgagttgagggtcaagttct |
| | | cccggtccgccgatgctcccgcctaccaacaggggcagaaccagctttataacgaactgaac |
| | | ctgggcaggagggaggaatatgatgtgttggataagcgccggggccgggacccagaaatg |
| | | gggggaaagcccagaagaaagaaccctcaagagggactttacaacgaattgcagaaagac |
| | | aaaatggccgaggcctactccgagattgggatgaagggcgaaagacggagaggaaaggg |
| | | gcacgacgggctctaccagggactcagcaccgccaccaaagatacctacgacgccctgcat |
| | | atgcaggcgctgccgccgcgc |
| Full length | 736 | MALPVTALLLPLALLLHAARPEVQLQQSGPGLVKPSQTLSL |
| CD19-CD22 | | TCAISGDSMLSNSDTWNWIRQSPSRGLEWLGRTYHRSTWY |
| Dual CAR | | DDYASSVRGRVSINVDTSKNQYSLQLNAVTPEDTGVYYCA |
| amino acid | | RVRLQDGNSWSDAFDVWGQGTMVTVSSGGGGSQSALTQP |
| | | ASASGSPGQSVTISCTGTSSDVGGYNYVSWYQQHPGKAPK |
| | | LMIYDVSNRPSGVSNRFSGSKSGNTASLTISGLQAEDEADY |
| | | YCSSYTSSSTLYVFGTGTQLTVLTTTPAPRPPTPAPTIASQPL |
| | | SLRPEACRPAAGGAVHTRGLDFACDIYIWAPLAGTCGVLL |
| | | LSLVITLYCKRGRKKLLYIFKQPFMRPVQTTQEEDGCSCRF |
| | | PEEEEGGCELRVKFSRSADAPAYQQGQNQLYNELNLGRRE |
| | | EYDVLDKRRGRDPEMGGKPRRKNPQEGLYNELQKDKMA |
| | | EAYSEIGMKGERRRGKGHDGLYQGLSTATKDTYDALHMQ |
| | | ALPPRGSGATNFSLLKQAGDVEENPGPMALPVTALLLPLAL |
| | | LLHAARPEIVMTQSPATLSLSPGERATLSCRASQDISKYLN |
| | | WYQQKPGQAPRLLIYHTSRLHSGIPARFSGSGSGTDYTLTIS |
| | | SLQPEDFAVYFCQQGNTLPYTFGQGTKLEIKGGGGSGGGG |
| | | SGGGGSQVQLQESGPGLVKPSETLSLTCTVSGVSLPDYGVS |
| | | WIRQPPGKGLEWIGVIWGSETTYYQSSLKSRVTISKDNSKN |
| | | QVSLKLSSVTAADTAVYYCAKHYYYGGSYAMDYWGQGT |
| | | LVTVSSTTTPAPRPPTPAPTIASQPLSLRPEACRPAAGGAVH |
| | | TRGLDFACDIYIWAPLAGTCGVLLLSLVITLYCKRGRKKLL |
| | | YIFKQPFMRPVQTTQEEDGCSCRFPEEEEGGCELRVKFSRS |
| | | ADAPAYQQGQNQLYNELNLGRREEYDVLDKRRGRDPEM |
| | | GGKPRRKNPQEGLYNELQKDKMAEAYSEIGMKGERRRGK |
| | | GHDGLYQGLSTATKDTYDALHMQALPPR |
| CD22 CAR | 737 | MALPVTALLLPLALLLHAARPEVQLQQSGPGLVKPSQTLSL |
| (with P2A site) | | TCAISGDSMLSNSDTWNWIRQSPSRGLEWLGRTYHRSTWY |
| | | DDYASSVRGRVSINVDTSKNQYSLQLNAVTPEDTGVYYCA |
| | | RVRLQDGNSWSDAFDVWGQGTMVTVSSGGGGSQSALTQP |
| | | ASASGSPGQSVTISCTGTSSDVGGYNYVSWYQQHPGKAPK |
| | | LMIYDVSNRPSGVSNRFSGSKSGNTASLTISGLQAEDEADY |
| | | YCSSYTSSSTLYVFGTGTQLTVLTTTPAPRPPTPAPTIASQPL |
| | | SLRPEACRPAAGGAVHTRGLDFACDIYIWAPLAGTCGVLL |
| | | LSLVITLYCKRGRKKLLYIFKQPFMRPVQTTQEEDGCSCRF |
| | | PEEEEGGCELRVKFSRSADAPAYQQGQNQLYNELNLGRRE |
| | | EYDVLDKRRGRDPEMGGKPRRKNPQEGLYNELQKDKMA |
| | | EAYSEIGMKGERRRGKGHDGLYQGLSTATKDTYDALHMQ |
| | | ALPPRGSGATNFSLLKQAGDVEENPG |
| CD19 CAR | 738 | PMALPVTALLLPLALLLHAARPEIVMTQSPATLSLSPGERA |
| | | TLSCRASQDISKYLNWYQQKPGQAPRLLIYHTSRLHSGIPA |
| | | RFSGSGSGTDYTLTISSLQPEDFAVYFCQQGNTLPYTFGQG |
| | | TKLEIKGGGGSGGGGSGGGGSQVQLQESGPGLVKPSETLSL |
| | | TCTVSGVSLPDYGVSWIRQPPGKGLEWIGVIWGSETTYYQS |
| | | SLKSRVTISKDNSKNQVSLKLSSVTAADTAVYYCAKHYYY |
| | | GGSYAMDYWGQGTLVTVSSTTTPAPRPPTPAPTIASQPLSL |
| | | RPEACRPAAGGAVHTRGLDFACDIYIWAPLAGTCGVLLLS |
| | | LVITLYCKRGRKKLLYIFKQPFMRPVQTTQEEDGCSCRFPE |
| | | EEEGGCELRVKFSRSADAPAYQQGQNQLYNELNLGRREEY |
| | | DVLDKRRGRDPEMGGKPRRKNPQEGLYNELQKDKMAEA |
| | | YSEIGMKGERRRGKGHDGLYQGLSTATKDTYDALHMQAL |
| | | PPR |

CD22 and CD19 CDRs of a dual CAR of the disclosure (e.g., a dual CAR molecule comprising (i) a first CAR that binds to CD22 and (ii) a second CAR that binds to CD19) are provided in Table 31.

TABLE 31
CD22 and CD19 CDR sequences

| | | |
|---|---|---|
| | SEQ | |
| Identifier | ID NO | Sequence |

CD22 CDRs

| | | |
|---|---|---|
| HCDR1 | 289 | SNSDTWN |
| (Kabat) | | |
| HCDR2 | 290 | RTYHRSTWYDDYASSVRG |
| (Kabat) | | |
| HCDR3 | 291 | VRLQDGNSWSDAFDV |
| (Kabat) | | |
| HCDR1 | 744 | GDSMLSNSD |
| (Chothia) | | |
| HCDR2 | 745 | YHRSTWY |
| (Chothia) | | |
| HCDR3 | 291 | VRLQDGNSWSDAFDV |
| (Chothia) | | |
| HCDR1 | 746 | GDSMLSNSDT |
| (IMGT) | | |
| HCDR2 | 747 | TYHRSTWYD |
| (IMGT) | | |
| HCDR3 | 748 | ARVRLQDGNSWSDAFDV |
| (IMGT) | | |
| LCDR1 | 95 | TGTSSDVGGYNYVS |
| (Kabat) | | |
| LCDR2 | 96 | DVSNRPS |
| (Kabat) | | |
| LCDR3 | 97 | SSYTSSSTLYV |
| (Kabat) | | |
| LCDR1 | 98 | TSSDVGGYNY |
| (Chothia) | | |
| LCDR2 | 99 | DVS |
| (Chothia) | | |
| LCDR3 | 100 | YTSSSTLY |
| (Chothia) | | |
| LCDR1 | 101 | SSDVGGYNY |
| (IMGT) | | |
| LCDR2 | 99 | DVS |
| (IMGT) | | |
| LCDR3 | 97 | SSYTSSSTLYV |
| (IMGT) | | |

CD19 CDRs

| | | |
|---|---|---|
| HCDR1 | 749 | GVSLPDYGVS |
| Kabat) | | |
| HCDR2 | 750 | VIWGSETTYYSSSLKS |
| (Kabat) | 304 | VIWGSETTYYQSSLKS |
| | 751 | VIWGSETTYYNSSLKS |
| HCDR3 | 297 | HYYYGGSYAMDY |
| (Kabat) | | |
| LCDR1 | 298 | RASQDISKYLN |
| LCDR2 | 299 | HTSRLHS |
| LCDR3 | 300 | QQGNTLPYT |

Table 32 provides nucleotide and amino acid sequence for CD19 and CD22 binding domains of a dual CAR or a tandem CAR disclosed herein, e.g., a dual CAR or a tandem CAR comprising (i) a first CAR that binds to CD22 and (ii) a second CAR that binds to CD19.

TABLE 32
CD19 and CD22 binding domains

| | SEQ ID | |
|---|---|---|
| Identifier | NO | Sequence |
| scFv | 752 | gaaattgtgatgacccagtcacccgccactcttagcctttcacccggtgagcgcgcaaccctgt |
| CAR19 in | | cttgcagagcctcccaagacatctcaaaataccttaattggtatcaacagaagcccggacaggc |
| c201, c203 | | tcctcgccttctgatctaccacaccagccggctccattctggaatccctgccaggttcagcggta |
| and tandem | | gcggatctgggaccgactacaccctcactatcagctcactgcagccagaggacttcgctgtcta |
| CARs c171, | | tttctgtcagcaagggaacaccctgccctacacctttggacagggcaccaagctcgagattaaa |
| c182, c188 | | ggtggaggtggcagcggaggaggtgggtccggcggtggaggaagccaggtccaactccaa |
| | | gaaagcggaccgggtcttgtgaagccatcagaaactctttcactgacttgtactgtgagcggag |
| | | tgtctctccccgattacggggtgtcttggatcagacagccaccggggaagggtctggaatggat |
| | | tggagtgatttggggctctgagactacttactaccaatcatccctcaagtcacgcgtcaccatctc |
| | | aaaggacaactctaagaatcaggtgtcactgaaactgtcatctgtgaccgcagccgacaccgc |
| | | cgtgtactattgcgctaagcattactattatggcgggagctacgcaatggattactggggacagg |
| | | gtactctggtcaccgtgtccagc |
| | 293 | EIVMTQSPATLSLSPGERATLSCRASQDISKYLNWYQQKPG |
| | | QAPRLLIYHTSRLHSGIPARFSGSGSGTDYTLTISSLQPEDFA |
| | | VYFCQQGNTLPYTFGQGTKLEIKGGGGSGGGGSGGGGSQV |
| | | QLQESGPGLVKPSETLSLTCTVSGVSLPDYGVSWIRQPPGKG |
| | | LEWIGVIWGSETTYYQSSLKSRVTISKDNSKNQVSLKLSSVT |
| | | AADTAVYYCAKHYYYGGSYAMDYWGQGTLVTVSS |
| scFv | 753 | gagattgtcatgactcagtccccggccacactctccctgtcacccggagaaagagcaaccctg |
| CAR19 in | | agctgcagggcgtcccaggacatctcgaagtacctgaactggtaccagcagaagcctggaca |
| c224 | | agcaccccgcctcctgatctaccacacctcgcggctgcattcgggaatccccgccagattctca |
| | | gggagcggatcaggaaccgactacaccctgactatctcgagcctgcaaccagaggatttcgc |
| | | cgtgtacttctgccagcaaggaaacaccctgccctacacctttggacagggaaccaagctcga |
| | | gattaaggggggggtggatcgggagggggtggatcaggaggaggcggctcacaagtcca |
| | | gctgcaagaatccggtccgggacttgtgaagccgtccgaaaccctgtcactgacttgcactgtg |
| | | tccggggtgtcattgcccgactacggcgtgagctggattcggcagccccctggaaagggattg |
| | | gaatggatcggcgtgatctggggttcggaaactacctactatcagtcctcactgaagtcccgcg |
| | | tgaccatcagcaaggataattccaaaaaccaagtgtctctgaagctctccagcgtcactgccgc |
| | | cgatactgccgtgtactactgcgccaagcactactattacggcggttcgtacgccatggactact |
| | | ggggccaagggacactcgtgaccgtgtcatcc |
| | 293 | EIVMTQSPATLSLSPGERATLSCRASQDISKYLNWYQQKPG |
| | | QAPRLLIYHTSRLHSGIPARFSGSGSGTDYTLTISSLQPEDFA |
| | | VYFCQQGNTLPYTFGQGTKLEIKGGGGSGGGGSGGGGSQV |
| | | QLQESGPGLVKPSETLSLTCTVSGVSLPDYGVSWIRQPPGKG |
| | | LEWIGVIWGSETTYYQSSLKSRVTISKDNSKNQVSLKLSSVT |
| | | AADTAVYYCAKHYYYGGSYAMDYWGQGTLVTVSS |
| scFv | 754 | caagtccagctgcaagaatccggtccgggacttgtgaagccgtccgaaaccctgtcactgactt |
| CAR19 in | | gcactgtgtccggggtgtcattgcccgactacggcgtgagctggattcggcagccccctggaa |
| c227 | | agggattggaatggatcggcgtgatctggggttcggaaactacctactatcagtcctcactgaa |
| | | gtcccgcgtgaccatcagcaaggataattccaaaaaccaagtgtctctgaagctctccagcgtc |
| | | actgccgccgatactgccgtgtactactgcgccaagcactactattacggcggttcgtacgccat |
| | | ggactactggggacaaggcactcttgtgactgtgtcaagcggcggtggagggagcggtggg |
| | | ggcggttcaggaggaggcggatcagagatcgtgatgacccaatccccagccaccctgtccct |
| | | cagccctggagaaagagccaccctgagctgccgggcctcccaggatatcagcaagtacttga |
| | | actggtaccaacaaaagccggggcaggcgccccggctcctgatctaccacacctcgcgcctc |
| | | cactcaggtatccccgccagattctcagggagcggctccggtactgactacaccctgactatttc |
| | | ctcactgcagccagaggactttgccgtgtacttctgccagcagggaaacactctgccgtacacc |
| | | ttcgggcagggaacgaagcttgaaattaag |
| | 755 | QVQLQESGPGLVKPSETLSLTCTVSGVSLPDYGVSWIRQPPG |
| | | KGLEWIGVIWGSETTYYQSSLKSRVTISKDNSKNQVSLKLSS |
| | | VTAADTAVYYCAKHYYYGGSYAMDYWGQGTLVTVSSGG |
| | | GGSGGGGSGGGGSEIVMTQSPATLSLSPGERATLSCRASQDI |
| | | SKYLNWYQQKPGQAPRLLIYHTSRLHSGIPARFSGSGSGTD |
| | | YTLTISSLQPEDFAVYFCQQGNTLPYTFGQGTKLEIK |
| scFv | 756 | gagattgtgatgactcagagcccggcgaccctgtccctgtcccccggggagagagcaaccct |
| CAR19 in | | gtcgtgccgggcctcccaagacatctcaaagtacctcaattggtatcagcagaagccaggaca |
| c230 | | ggctccacggttgctgatctaccacacttcgagactgcactcaggaatccccgcgcggttttcc |
| | | ggttccggctccgggaccgactacaccctgaccatcagctcgctccagcctgaggatttcgca |
| | | gtgtacttctgtcagcaaggaaacacccttccatacaccttcggacagggtaccaagctggaaa |
| | | tcaagggaggaggaggatctgggggcggtggttccggaggcggtggaagccaagtgcagc |
| | | tccaggaaagcggacccgggctggtcaagccgagcgaaaccctctcactgacttgtactgtgt |
| | | ccggagtgtccctgcctgactatggagtgtcctggatccgacagccccccggaaagggtctgg |
| | | agtggattggggtcatctggggctccgaaactacctactaccagagcagcctcaagagccgg |
| | | gtcaccatttcaaaggataactccaagaatcaagtgtccctgaagctgtcctcagtgacagccg |
| | | cagacaccgccgtgtactactgcgccaagcactactactacggaggctcctacgcaatggact |
| | | actggggacaaggcactttggtcactgtgtcaagc |
| | 293 | EIVMTQSPATLSLSPGERATLSCRASQDISKYLNWYQQKPG |
| | | QAPRLLIYHTSRLHSGIPARFSGSGSGTDYTLTISSLQPEDFA |
| | | VYFCQQGNTLPYTFGQGTKLEIKGGGGSGGGGSGGGGSQV |
| | | QLQESGPGLVKPSETLSLTCTVSGVSLPDYGVSWIRQPPGKG |
| | | LEWIGVIWGSETTYYQSSLKSRVTISKDNSKNQVSLKLSSVT |
| | | AADTAVYYCAKHYYYGGSYAMDYWGQGTLVTVSS |
| ScFVCAR2 | 757 | gaagtgcagctgcagcagtcagggcctggcctggtcaagccgtcgcagaccctctccctgac |
| 2 in c201 | | atgcgccattagcggggactccatgctgagcaactcggacacctggaactggattcggcagtc |
| and c203 | | cccttcccggggactcgagtggctcggacgcacctaccatcggagcacttggtacgacgacta |
| | | cgcctcctccgtgagaggtcgcgtgtcgatcaacgtggatacctcgaagaaccagtatagcttg |
| | | caactgaacgccgtgacccctgaggataccggagtgtactattgtgcgagagtcaggctgcaa |
| | | gacggaaactcctggtccgacgcatttgatgtctggggacagggtactatggtcacggtgtcat |
| | | ctggaggcggaggatcgcaaagcgccctgactcagccggcttcggctagcggttcaccggg |
| | | gcagtccgtgactatctcctgcaccgggacttcctccgacgtgggaggctacaattacgtgtcct |
| | | ggtaccagcaacaccccggcaaagccccaaagctgatgatctacgacgtcagcaacagacc |
| | | cagcggagtgtccaaccggttcagcggctccaagtccggcaacaccgcctccctgaccatca |
| | | gcgggcttcaggccgaagatgaggcggattactactgctcctcgtacacctcaagctcaactct |
| | | gtacgtgttcggcaccggtactcagctcaccgtgctg |
| | 758 | EVQLQQSGPGLVKPSQTLSLTCAISGDSMLSNSDTWNWIRQ |
| | | SPSRGLEWLGRTYHRSTWYDDYASSVRGRVSINVDTSKNQ |
| | | YSLQLNAVTPEDTGVYYCARVRLQDGNSWSDAFDVWGQG |
| | | TMVTVSSGGGGSQSALTQPASASGSPGQSVTISCTGTSSDVG |
| | | GYNYVSWYQQHPGKAPKLMIYDVSNRPSGVSNRFSGSKSG |
| | | NTASLTISGLQAEDEADYYCSSYTSSSTLYVFGTGTQLTVL |
| ScFVCAR2 | 759 | gaagtgcagctgcagcagtcaggaccgggcctggtcaaaccttcgcagactctgtccctgact |
| 2 in 230 | | tgcgctataagcggggactccatgctgagcaattcggacacttggaactggattcgccaaagc |
| | | cccagccggggtctggaatggctgggaaggacctaccatcgctctacttggtacgacgactac |
| | | gccagctccgtgcgaggacgcgtgtccatcaacgtggacacctccaagaaccagtactcgctt |
| | | caactcaacgcagtgacccctgaagataccggagtctactattgcgcccgcgtgcggctccag |
| | | gacgggaactcctggtcggacgctttcgatgtctggggacagggcactatggtcaccgtcagc |
| | | tccggcggcggcggtagccaatcggcgctgacacagccggcttccgcctcgggatcgcctg |
| | | gacagtcggtgaccatctcgtgcactggaacctcctccgacgtgggcggctacaattatgtgtc |
| | | atggtaccagcagcacccgggaaaggcccctaagctgatgatctacgacgtgtccaatagacc |
| | | tagcggggtgtcaaacagattctccggatccaaatccggaaacactgcctccctgaccatttcc |
| | | ggactgcaggccgaggacgaagccgattactactgctcctcttacacctcctcatccaccctcta |
| | | cgtgtttgggactgggacccagctgaccgtcctc |
| | 758 | EVQLQQSGPGLVKPSQTLSLTCAISGDSMLSNSDTWNWIRQ |
| | | SPSRGLEWLGRTYHRSTWYDDYASSVRGRVSINVDTSKNQ |
| | | YSLQLNAVTPEDTGVYYCARVRLQDGNSWSDAFDVWGQG |
| | | TMVTVSSGGGGSQSALTQPASASGSPGQSVTISCTGTSSDVG |
| | | GYNYVSWYQQHPGKAPKLMIYDVSNRPSGVSNRFSGSKSG |
| | | NTASLTISGLQAEDEADYYCSSYTSSSTLYVFGTGTQLTVL |
| ScFVCAR2 | 760 | gaagtgcagcttcaacaatcaggaccaggactcgtcaaaccatcacagaccctctccctcacat |
| 2 in 171, | | gtgccatctccggggactccatgttgagcaattccgacacttggaattggattagacaaagccc |
| c182 | | gtcccggggtctggaatggttgggacgcacctaccaccggtctacttggtacgacgactacgc |
| | | gtcatccgtgcggggaagagtgtccatcaacgtggacacctccaagaaccagtacagcctgc |
| | | agcttaatgccgtgactcctgaggatacgggcgtctactactgcgcccgcgtccgcctgcaag |
| | | acgggaacagctggagcgatgcattcgatgtctggggccagggaactatggtcaccgtgtcgt |
| | | ctgggggcggtggatcgggtggcgggggttcggggggcggcggctctcagtccgctcttac |
| | | ccaaccggcctcagcctcggggagccccggccagagcgtgaccatttcctgcaccggcactt |
| | | catccgacgtgggcggctacaactacgtgtcctggtaccaacagcacccgggaaaggccccc |
| | | aagctcatgatctacgacgtgtccaacaggccctcgggagtgtccaaccggttctcgggttcga |
| | | aatcgggaaacacagccagcctgaccatcagcggactgcaggctgaagatgaagccgacta |
| | | ctactgctcctcctacacctcgtcatccacgctctacgtgttcggcactggaactcagctgactgt |
| | | gctg |
| | 285 | EVQLQQSGPGLVKPSQTLSLTCAISGDSMLSNSDTWNWIRQ |
| | | SPSRGLEWLGRTYHRSTWYDDYASSVRGRVSINVDTSKNQ |
| | | YSLQLNAVTPEDIGVYYCARVRLQDGNSWSDAFDVWGQG |
| | | TMVTVSSGGGGSGGGGSGGGGSQSALTQPASASGSPGQSV |
| | | TISCTGTSSDVGGYNYVSWYQQHPGKAPKLMIYDVSNRPSG |
| | | VSNRFSGSKSGNTASLTISGLQAEDEADYYCSSYTSSSTLYV |
| | | FGTGTQLTVL |
| ScFVCAR2 | 761 | cagtccgctcttacccaaccggcctcagcctcggggagccccggccagagcgtgaccatttcc |
| 2 in c188 | | tgcaccggcacttcatccgacgtgggcggctacaactacgtgtcctggtaccaacagcacccg |
| | | ggaaaggcccccaagctcatgatctacgacgtgtccaacaggccctcgggagtgtccaaccg |
| | | gttctcgggttcgaaatcgggaaacacagccagcctgaccatcagcggactgcaggctgaag |
| | | atgaagccgactactactgctcctcctacacctcgtcatccacgctctacgtgttcggcactgga |
| | | actcagctgactgtgctgggcggaggaggctccgaagtgcagcttcaacaatcaggaccagg |
| | | actcgtcaaaccatcacagaccctctccctcacatgtgccatctccggggactccatgttgagca |
| | | attccgacacttggaattggattagacaaagcccgtcccggggtctggaatggttgggacgca |
| | | cctaccaccggtctacttggtacgacgactacgcgtcatccgtgcggggaagagtgtccatcaa |
| | | cgtggacacctccaagaaccagtacagcctgcagcttaatgccgtgactcctgaggatacggg |
| | | cgtctactactgcgcccgcgtccgcctgcaagacgggaacagctggagcgatgcattcgatgt |
| | | ctggggccagggaactatggtcaccgtgtcgtct |
| | 762 | QSALTQPASASGSPGQSVTISCTGTSSDVGGYNYVSWYQQH |
| | | PGKAPKLMIYDVSNRPSGVSNRFSGSKSGNTASLTISGLQAE |
| | | DEADYYCSSYTSSSTLYVFGTGTQLTVLGGGGSEVQLQQSG |
| | | PGLVKPSQTLSLTCAISGDSMLSNSDTWNWIRQSPSRGLEW |
| | | LGRTYHRSTWYDDYASSVRGRVSINVDTSKNQYSLQLNAV |
| | | TPEDTGVYYCARVRLQDGNSWSDAFDVWGQGTMVTVSS |
| ScFVCAR2 | 763 | cagtccgctctgactcagccggcctcagcttcggggtcccctggtcaaagcgtcactatttcctg |
| 2 in c224 | | taccggaacctcatcagacgtgggcggctacaattacgtgtcctggtaccaacagcaccccgg |
| | | aaaggctcctaagcttatgatctacgacgtgtccaaccggccgtcaggagtgtccaacagattct |
| | | ccggctccaagagcggaaacactgccagcttgaccattagcggcttgcaggccgaggacga |
| | | agccgactactactgctctagctacacatcctcgtctaccctctacgtgtttggaacggggaccc |
| | | agctgactgtgctcgggggtggaggatcagaggtgcaactccagcagtccggtcctggcctc |
| | | gtgaaaccgtcccaaaccctgtccctgacttgcgccatctcgggcgactccatgctgtccaattc |
| | | cgacacctggaactggattagacaatcgcctagccggggactcgaatggctgggccggacct |
| | | accaccggtccacgtggtatgacgactacgcaagctccgtccggggaagggtgtccattaacg |
| | | tcgatacctccaagaaccagtacagccttcagctgaacgctgtgacccccgaggataccggcg |
| | | tctactactgtgcaagagtgcgattgcaggatggaaactcgtggtcggacgcattcgatgtctg |
| | | gggacagggaactatggtgaccgtgtcctcg |
| | 762 | QSALTQPASASGSPGQSVTISCTGTSSDVGGYNYVSWYQQH |
| | | PGKAPKLMIYDVSNRPSGVSNRFSGSKSGNTASLTISGLQAE |
| | | DEADYYCSSYTSSSTLYVFGTGTQLTVLGGGGSEVQLQQSG |
| | | PGLVKPSQTLSLTCAISGDSMLSNSDTWNWIRQSPSRGLEW |
| | | LGRTYHRSTWYDDYASSVRGRVSINVDTSKNQYSLQLNAV |
| | | TPEDTGVYYCARVRLQDGNSWSDAFDVWGQGTMVTVSS |
| ScFVCAR2 | 764 | cagtccgctctgactcagccggcctcagcttcggggtcccctggtcaaagcgtcactatttcctg |
| 2 in c227 | | taccggaacctcatcagacgtgggcggctacaattacgtgtcctggtaccaacagcaccccgg |
| | | aaaggctcctaagcttatgatctacgacgtgtccaaccggccgtcaggagtgtccaacagattct |
| | | ccggctccaagagcggaaacactgccagcttgaccattagcggcttgcaggccgaggacga |
| | | agccgactactactgctctagctacacatcctcgtctaccctctacgtgtttggaacggggaccc |
| | | agctgactgtgctcgggggtggaggatcagaggtgcaactccagcagtccggtcctggcctc |
| | | gtgaaaccgtcccaaaccctgtccctgacttgcgccatctcgggcgactccatgctgtccaattc |
| | | cgacacctggaactggattagacaatcgcctagccggggactcgaatggctgggccggacct |
| | | accaccggtccacgtggtatgacgactacgcaagctccgtccggggaagggtgtccattaacg |
| | | tcgatacctccaagaaccagtacagccttcagctgaacgctgtgacccccgaggataccggcg |
| | | tctactactgtgcaagagtgcgattgcaggatggaaactcgtggtcggacgcattcgatgtctg |
| | | gggacagggaactatggtcactgtgtcctcc |
| | 762 | QSALTQPASASGSPGQSVTISCTGTSSDVGGYNYVSWYQQH |
| | | PGKAPKLMIYDVSNRPSGVSNRFSGSKSGNTASLTISGLQAE |
| | | DEADYYCSSYTSSSTLYVFGTGTQLTVLGGGGSEVQLQQSG |
| | | PGLVKPSQTLSLTCAISGDSMLSNSDTWNWIRQSPSRGLEW |
| | | LGRTYHRSTWYDDYASSVRGRVSINVDTSKNQYSLQLNAV |
| | | TPEDTGVYYCARVRLQDGNSWSDAFDVWGQGTMVTVSS |

Table 33 provides nucleotide and amino acid sequences for additional CAR components, e.g., signal peptide, linkers and P2A sites, that can be used in a CAR molecule, e.g., a dual CAR molecule described herein (for example, a dual CAR molecule comprising (i) a first CAR that binds to CD22 and (ii) a second CAR that binds to CD19).

TABLE 33
Additional CAR components

| | SEQ | |
|---|---|---|
| Identifier | ID NO | Sequence |
| Signal peptide for | 199 | atggccctccctgtcaccgccctgctgcttccgctggctcttctgctccacgccgctcggc |
| CAR22 in c201, | | cc |
| c203, and tandem | 1 | MALPVTALLLPLALLLHAARP |
| CARs c171, | | |
| c182, c188 | | |
| Signal peptide | 765 | atggccctgcccgtgactgcgctcctgcttccgttggccctgctcctgcatgccgccagac |
| in tandem CARs | | ct |
| c224, c227 | 1 | MALPVTALLLPLALLLHAARP |
| Signal peptide | 766 | atggccttaccagtgaccgccttgctcctgccgctggccttgctgctccacgccgccagg |
| CAR19 in c201 | | ccg |
| and c203 | 1 | MALPVTALLLPLALLLHAARP |
| Signal peptide | 767 | atggcacttcccgtcaccgccctgctgctcccactcgccctccttctgcacgccgcccgcc |
| CAR22 in c230 | | cc |
| | 1 | MALPVTALLLPLALLLHAARP |
| Signal peptide | 768 | atggccctgccagtgaccgcgctcctgctgcccctggctctgctgcttcacgcggcccgg |
| CAR19 in c230 | | cct |
| | 1 | MALPVTALLLPLALLLHAARP |
| CD8 hinge and | 769 | accactaccccagcaccgaggccacccaccccggctcctaccatcgcctcccagcctct |
| transmembrane | | gtccctgcgtccggaggcatgtagacccgcagctggtggggccgtgcatacccggggt |
| CAR22 in c201 | | cttgacttcgcctgcgatatctacatttgggcccctctggctggtacttgcggggtcctgctg |
| and c203, and in | | ctttcactcgtgatcactctttactgt |
| tandem CARs | 202 | TTTPAPRPPTPAPTIASQPLSLRPEACRPAAGGAVHTRGL |
| c171, c182, c188, | | DFACDIYIWAPLAGTCGVLLLSLVITLYC |
| c224, c227 | | |
| CD8 hinge and | 770 | actaccaccccggccccgcggccccctacaccggcaccgactattgccagccagcctct |
| transmembrane | | ctcgctgcggccggaggcctgccgcccagccgccggcggagccgtgcacacccgcg |
| CAR22 | | gtctggacttcgcgtgcgatatctacatctgggctccgctggccgggacttgtggcgtgct |
| In c230 | | gctgctgtctctggtcatcacactgtactgc |
| | 202 | TTTPAPRPPTPAPTIASQPLSLRPEACRPAAGGAVHTRGL |
| | | DFACDIYIWAPLAGTCGVLLLSLVITLYC |
| CD8 hinge and | 771 | accacgacgccagcgccgcgaccaccaacaccggcgcccaccatcgcgtcgcagccc |
| transmembrane | | ctgtccctgcgcccagaggcgtgccggccagcggcggggggcgcagtgcacacgag |
| CAR19 in c201 | | ggggctggacttcgcctgtgatatctacatctgggcgcccttggccgggacttgtggggtc |
| and c203 | | cttctcctgtcactggttatcaccctttactgc |
| | 202 | TTTPAPRPPTPAPTIASQPLSLRPEACRPAAGGAVHTRGL |
| | | DFACDIYIWAPLAGTCGVLLLSLVITLYC |
| CD8 hinge and | 772 | accaccacccctgcgcctcggcctcctaccccggctcccactatcgcgagccagccgct |
| transmembrane | | gagcctgcggcctgaggcttgccgaccggccgctggcggcgccgtgcatactcgggg |
| CAR19 | | cctcgactttgcctgtgacatctacatctgggcccccctggccggaacgtgcggagtgct |
| In c230 | | gctgctgtcgctggtcattaccctgtattgc |
| | 202 | TTTPAPRPPTPAPTIASQPLSLRPEACRPAAGGAVHTRGL |
| | | DFACDIYIWAPLAGTCGVLLLSLVITLYC |
| 4-1BB | 773 | aagcgcggtcggaagaagctgctgtacatctttaagcaacccttcatgaggcctgtgcag |
| CAR22 in c201 | | actactcaagaggaggacggctgttcatgccggttcccagaggaggaggaaggcggct |
| and c203, and | | gcgaactg |
| tandem CARs | 7 | KRGRKKLLYIFKQPFMRPVQTTQEEDGCSCRFPEEEEGG |
| c171, c182, c188, | | CEL |
| c224, c227 | | |
| 4-1BB | 18 | aaacggggcagaaagaaactcctgtatatattcaaacaaccatttatgagaccagtacaaa |
| CAR19 in c201 | | ctactcaagaggaagatggctgtagctgccgatttccagaagaagaagaaggaggatgt |
| and c203 | | gaactg |
| | 7 | KRGRKKLLYIFKQPFMRPVQTTQEEDGCSCRFPEEEEGG |
| | | CEL |
| 4-1BB | 774 | aagcgcggaagaaagaagctgctctacatcttcaagcaacccttcatgcggcctgtgcag |
| CAR22 in c230 | | accacccaggaagaggatggctgctcctgccggttcccggaggaagaagagggcgga |
| | | tgcgaactg |
| | 7 | KRGRKKLLYIFKQPFMRPVQTTQEEDGCSCRFPEEEEGG |
| | | CEL |
| 4-1BB | 775 | aaacgcggaaggaagaagctgttgtacattttcaagcagcccttcatgcgcccggtgcaa |
| CAR19 in c230 | | actactcaggaggaagatggctgttcctgtcggttccccgaagaggaagaaggcggctg |
| | | cgagttg |
| | 7 | KRGRKKLLYIFKQPFMRPVQTTQEEDGCSCRFPEEEEGG |
| | | CEL |
| CD3zeta | 776 | cgcgtgaaattcagccgcagcgcagatgctccagcctaccagcaggggcagaaccagc |
| CAR22 in c201 | | tctacaacgaactcaatcttggtcggagagaggagtacgacgtgctggacaagcggaga |
| and c203, and | | ggacgggacccagaaatggggggaagccgcgcagaaagaatccccaagagggcct |
| tandem CARs | | gtacaacgagctccaaaaggataagatggcagaagcctatagcgagattggtatgaaag |
| 171, c182, c188, | | gggaacgcagaagaggcaaaggccacgacggactgtaccagggactcagcaccgcc |
| c224, c227 | | accaaggacacctatgacgctcttcacatgcaggccctgccgcctcgg |
| | 10 | RVKFSRSADAPAYQQGQNQLYNELNLGRREEYDVLDKR |
| | | RGRDPEMGGKPRRKNPQEGLYNELQKDKMAEAYSEIG |
| | | MKGERRRGKGHDGLYQGLSTATKDTYDALHMQALPPR |
| CD3zeta | 21 | agagtgaagttcagcaggagcgcagacgcccccgcgtaccagcagggccagaaccag |
| CAR19 in c201 | | ctctataacgagctcaatctaggacgaagagaggagtacgatgttttggacaagagacgt |
| and c203 | | ggccgggaccctgagatggggggaaagccgagaaggaagaaccctcaggaaggcct |
| | | gtacaatgaactgcagaaagataagatggcggaggcctacagtgagattgggatgaaag |
| | | gcgagcgccggaggggcaaggggcacgatggcctttaccagggtctcagtacagcca |
| | | ccaaggacacctacgacgcccttcacatgcaggccctgccccctcgc |
| | 10 | RVKFSRSADAPAYQQGQNQLYNELNLGRREEYDVLDKR |
| | | RGRDPEMGGKPRRKNPQEGLYNELQKDKMAEAYSEIG |
| | | MKGERRRGKGHDGLYQGLSTATKDTYDALHMQALPPR |
| CD3zeta | 777 | cgcgtgaagttcagccgaagcgccgacgccccggcctaccagcagggccagaaccaa |
| CAR22 in c230 | | ctgtacaacgaactcaacctgggtcggagagaagagtacgacgtgctggacaaaagac |
| | | gcggcagggaccccgagatgggcggaaagcctcgccgcaagaacccgcaggaggg |
| | | cctctacaacgagctgcagaaggacaagatggccgaagcctactcagagatcggcatg |
| | | aagggggagcggaggcgcgggaagggccacgacggtttgtaccaaggactttccact |
| | | gcgaccaaggacacctacgatgccctccatatgcaagccctgccgccccgg |
| | 10 | RVKFSRSADAPAYQQGQNQLYNELNLGRREEYDVLDKR |
| | | RGRDPEMGGKPRRKNPQEGLYNELQKDKMAEAYSEIG |
| | | MKGERRRGKGHDGLYQGLSTATKDTYDALHMQALPPR |
| CD3zeta | 778 | agggtcaagttctcccggtccgccgatgctcccgcctaccaacaggggcagaaccagct |
| CAR19 in c230 | | ttataacgaactgaacctgggcaggagggaggaatatgatgtgttggataagcgccggg |
| | | gccgggacccagaaatggggggaaagcccagaagaaagaaccctcaagagggacttt |
| | | acaacgaattgcagaaagacaaaatggccgaggcctactccgagattgggatgaaggg |
| | | cgaaagacggagaggaaaggggcacgacgggctctaccagggactcagcaccgcca |
| | | ccaaagatacctacgacgccctgcatatgcaggcgctgccgccgcgc |
| | 10 | RVKFSRSADAPAYQQGQNQLYNELNLGRREEYDVLDKR |
| | | RGRDPEMGGKPRRKNPQEGLYNELQKDKMAEAYSEIG |
| | | MKGERRRGKGHDGLYQGLSTATKDTYDALHMQALPPR |
| Linker between | 779 | ttggcagaagccgccgcgaaa |
| scFVs in c171 | 308 | LAEAAAK |
| Linker between | 780 | ggtggaggtggcagcggaggaggtgggtccggcggtggaggaagc |
| scFVs in | | |
| c182, c188 | 104 | GGGGSGGGGSGGGGS |
| Linker between | 781 | ggcggaggcgggagcggaggaggaggctctggcggaggaggaagc |
| scFVs in c224 | 104 | GGGGSGGGGSGGGGS |
| Linker between | 782 | ggcggtggaggctcgggggggggcggctcaggaggaggcggctca |
| scFVs in c227 | 104 | GGGGSGGGGSGGGGS |
| P2A in c201, | 783 | ggaagcggagctactaacttcagcctgctgaagcaggctggagacgtggaggagaacc |
| c203 | | ctggacct |
| | 784 | GSGATNFSLLKQAGDVEENPGP |
| P2A in c230 | 785 | ggttccggagctaccaacttctcgctgttgaagcaggccggagatgtcgaggaaaaccc |
| | | gggacct |
| | 784 | GSGATNFSLLKQAGDVEENPGP |
| Gly4Ser linker | 786 | Ggtggaggtggcagc |
| | 25 | GGGGS |

In some embodiments, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. Placement of a costimulatory signaling domain, for example, 4-1BB, CD28, CD27, OX-40 or ICOS, onto the first CAR, and the primary signaling domain, for example, CD3 zeta, on the second CAR can limit the CAR activity to cells where both targets are expressed. In some embodiments, the CAR expressing cell comprises a first CAR that includes an antigen binding domain, a transmembrane domain and a costimulatory domain and a second CAR that targets another antigen and includes an antigen binding domain, a transmembrane domain and a primary signaling domain. In some embodiments, the CAR expressing cell comprises a first CAR that includes an antigen binding domain, a transmembrane domain and a primary signaling domain and a second CAR that targets another antigen and includes an antigen binding domain to the antigen, a transmembrane domain and a costimulatory signaling domain.

In some embodiments, the CAR-expressing cell comprises an XCAR described herein and an inhibitory CAR. In some embodiments, the inhibitory CAR comprises an antigen binding domain that binds an antigen found on normal cells but not cancer cells, for example, normal cells that also express X. In some embodiments, the inhibitory CAR comprises the antigen binding domain, a transmembrane domain and an intracellular domain of an inhibitory molecule. For example, the intracellular domain of the inhibitory CAR can be an intracellular domain of PD1, PD-L1, PD-L2, CTLA4, TIM3, CEACAM (CEACAM-1, CEACAM-3, and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (for example, TGF beta).

In some embodiments, when the CAR-expressing cell comprises two or more different CARs, the antigen binding domains of the different CARs can be such that the antigen binding domains do not interact with one another. For example, a cell expressing a first and second CAR can have an antigen binding domain of the first CAR, for example, as a fragment, for example, an scFv, that does not form an association with the antigen binding domain of the second CAR, for example, the antigen binding domain of the second CAR is a VHH.

In some embodiments, the antigen binding domain comprises a single domain antigen binding (SDAB) molecules include molecules whose complementary determining regions are part of a single domain polypeptide. Examples include, but are not limited to, heavy chain variable domains, binding molecules naturally devoid of light chains, single domains derived from conventional 4-chain antibodies, engineered domains and single domain scaffolds other than those derived from antibodies. SDAB molecules may be any of the art, or any future single domain molecules. SDAB molecules may be derived from any species including, but not limited to mouse, human, camel, llama, lamprey, fish, shark, goat, rabbit, and bovine. This term also includes naturally occurring single domain antibody molecules from species other than Camelidae and sharks.

In some embodiments, an SDAB molecule can be derived from a variable region of the immunoglobulin found in fish, such as, for example, that which is derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909.

In some embodiments, an SDAB molecule is a naturally occurring single domain antigen binding molecule known as heavy chain devoid of light chains. Such single domain molecules are disclosed in WO 9404678 and Hamers-Casterman, C. et al. (1993) Nature 363:446-448, for example. For clarity reasons, this variable domain derived from a heavy chain molecule naturally devoid of light chain is known herein as a VHH or nanobody to distinguish it from the conventional VH of four chain immunoglobulins. Such a VHH molecule can be derived from Camelidae species, for example in camel, llama, dromedary, alpaca and guanaco. Other species besides Camelidae may produce heavy chain molecules naturally devoid of light chain; such VHHs are within the scope of the invention.

The SDAB molecules can be recombinant, CDR-grafted, humanized, camelized, de-immunized and/or in vitro generated (for example, selected by phage display).

It has also been discovered, that cells having a plurality of chimeric membrane embedded receptors comprising an antigen binding domain that interactions between the antigen binding domain of the receptors can be undesirable, for example, because it inhibits the ability of one or more of the antigen binding domains to bind its cognate antigen. Accordingly, disclosed herein are cells having a first and a second non-naturally occurring chimeric membrane embedded receptor comprising antigen binding domains that minimize such interactions. Also disclosed herein are nucleic acids encoding a first and a second non-naturally occurring chimeric membrane embedded receptor comprising an antigen binding domains that minimize such interactions, as well as methods of making and using such cells and nucleic acids. In some embodiments the antigen binding domain of one of the first and the second non-naturally occurring chimeric membrane embedded receptor, comprises an scFv, and the other comprises a single VH domain, for example, a camelid, shark, or lamprey single VH domain, or a single VH domain derived from a human or mouse sequence.

In some embodiments, a composition herein comprises a first and second CAR, wherein the antigen binding domain of one of the first and the second CAR does not comprise a variable light domain and a variable heavy domain. In some embodiments, the antigen binding domain of one of the first and the second CAR is an scFv, and the other is not an scFv. In some embodiments, the antigen binding domain of one of the first and the second CAR comprises a single VH domain, for example, a camelid, shark, or lamprey single VH domain, or a single VH domain derived from a human or mouse sequence. In some embodiments, the antigen binding domain of one of the first and the second CAR comprises a nanobody. In some embodiments, the antigen binding domain of one of the first and the second CAR comprises a camelid VHH domain.

In some embodiments, the antigen binding domain of one of the first and the second CAR comprises an scFv, and the other comprises a single VH domain, for example, a camelid, shark, or lamprey single VH domain, or a single VH domain derived from a human or mouse sequence. In some embodiments, the antigen binding domain of one of the first and the second CAR comprises an scFv, and the other comprises a nanobody. In some embodiments, the antigen binding domain of one of the first and the second CAR comprises an scFv, and the other comprises a camelid VHH domain.

In some embodiments, when present on the surface of a cell, binding of the antigen binding domain of the first CAR to its cognate antigen is not substantially reduced by the presence of the second CAR. In some embodiments, binding of the antigen binding domain of the first CAR to its cognate antigen in the presence of the second CAR is at least 85%, 90%, 95%, 96%, 97%, 98% or 99%, for example, 85%, 90%, 95%, 96%, 97%, 98% or 99% of binding of the antigen binding domain of the first CAR to its cognate antigen in the absence of the second CAR.

In some embodiments, when present on the surface of a cell, the antigen binding domains of the first and the second CAR, associate with one another less than if both were scFv antigen binding domains. In some embodiments, the antigen binding domains of the first and the second CAR, associate with one another at least 85%, 90%, 95%, 96%, 97%, 98% or 99% less than, for example, 85%, 90%, 95%, 96%, 97%, 98% or 99% less than if both were scFv antigen binding domains.

Co-Expression of an Agent that Enhances CAR Activity

In some embodiments, the CAR-expressing cell described herein can further express another agent, for example, an agent that enhances the activity or fitness of a CAR-expressing cell.

For example, in some embodiments, the agent can be an agent which inhibits a molecule that modulates or regulates, for example, inhibits, T cell function. In some embodiments, the molecule that modulates or regulates T cell function is an inhibitory molecule. Inhibitory molecules, for example, PD1, can, in some embodiments, decrease the ability of a CAR-expressing cell to mount an immune effector response. Examples of inhibitory molecules include PD1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, or TGF beta.

In embodiments, an agent, for example, an inhibitory nucleic acid, for example, a dsRNA, for example, an siRNA or shRNA; or for example, an inhibitory protein or system, for example, a clustered regularly interspaced short palindromic repeats (CRISPR), a transcription-activator like effector nuclease (TALEN), or a zinc finger endonuclease (ZFN), for example, as described herein, can be used to inhibit expression of a molecule that modulates or regulates, for example, inhibits, T-cell function in the CAR-expressing cell. In some embodiments the agent is an shRNA, for example, an shRNA described herein. In some embodiments, the agent that modulates or regulates, for example, inhibits, T-cell function is inhibited within a CAR-expressing cell. For example, a dsRNA molecule that inhibits expression of a molecule that modulates or regulates, for example, inhibits, T-cell function is linked to the nucleic acid that encodes a component, for example, all of the components, of the CAR.

In some embodiments, the agent which inhibits an inhibitory molecule comprises a first polypeptide, for example, an inhibitory molecule, associated with a second polypeptide that provides a positive signal to the cell, for example, an intracellular signaling domain described herein. In some embodiments, the agent comprises a first polypeptide, for example, of an inhibitory molecule such as PD1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, or TGF beta, or a fragment of any of these (for example, at least a portion of an extracellular domain of any of these), and a second polypeptide which is an intracellular signaling domain described herein (for example, comprising a costimulatory domain (for example, 41BB, CD27 or CD28, for example, as described herein) and/or a primary signaling domain (for example, a CD3 zeta signaling domain described herein). In some embodiments, the agent comprises a first polypeptide of PD1 or a fragment thereof (for example, at least a portion of an extracellular domain of PD1), and a second polypeptide of an intracellular signaling domain described herein (for example, a CD28 signaling domain described herein and/or a CD3 zeta signaling domain described herein). PD1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD1, PD-L1 and PD-L2 have been shown to downregulate T cell activation upon binding to PD1 (Freeman et a. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD1 with PD-L1.

In some embodiments, the agent comprises the extracellular domain (ECD) of an inhibitory molecule, for example, Programmed Death 1 (PD1), can be fused to a transmembrane domain and intracellular signaling domains such as 41BB and CD3 zeta (also referred to herein as a PD1 CAR). In some embodiments, the PD1 CAR, when used in combinations with an XCAR described herein, improves the persistence of the T cell. In some embodiments, the CAR is a PD1 CAR comprising the extracellular domain of PD1 indicated as underlined in SEQ ID NO: 24. In some embodiments, the PD1 CAR comprises the amino acid sequence of SEQ ID NO: 24.

In some embodiments, the PD1 CAR comprises the amino acid sequence of SEQ ID NO: 22.

In some embodiments, the agent comprises a nucleic acid sequence encoding the PD1 CAR, for example, the PD1 CAR described herein. In some embodiments, the nucleic acid sequence for the PD1 CAR is provided as SEQ ID NO: 23, with the PD1 ECD underlined.

In another example, in some embodiments, the agent which enhances the activity of a CAR-expressing cell can be a costimulatory molecule or costimulatory molecule ligand. Examples of costimulatory molecules include MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, for example, as described herein. Examples of costimulatory molecule ligands include CD80, CD86, CD40L, ICOSL, CD70, OX40L, 4-1BBL, GITRL, and LIGHT. In embodiments, the costimulatory molecule ligand is a ligand for a costimulatory molecule different from the costimulatory molecule domain of the CAR. In embodiments, the costimulatory molecule ligand is a ligand for a costimulatory molecule that is the same as the costimulatory molecule domain of the CAR. In some embodiments, the costimulatory molecule ligand is 4-1BBL. In some embodiments, the costimulatory ligand is CD80 or CD86. In some embodiments, the costimulatory molecule ligand is CD70. In embodiments, a CAR-expressing immune effector cell described herein can be further engineered to express one or more additional costimulatory molecules or costimulatory molecule ligands.

TET2 shRNA

For example, in some embodiments, the agent can be an agent which inhibits expression of a target that enhances CAR-T cell function—for example, by (i) enhancing proliferation and/or (ii) regulating effector function through regulation of cytokine production and/or degranulation. In some embodiments, an agent, for example, an inhibitory nucleic acid, for example, a dsRNA, for example, an siRNA or shRNA; or for example, an inhibitory protein or system, for example, a clustered regularly interspaced short palindromic repeats (CRISPR), a transcription-activator like effector nuclease (TALEN), or a zinc finger endonuclease (ZFN), for example, as described herein, can be used to inhibit expression of a target that enhances CAR T-cell function. In some embodiments, the target is Tet2. In some embodiments the agent is an shRNA, for example, an shRNA that targets Tet2, comprising a sense strand comprising a Tet2 target sequence and an anti-sense strand complementary to the sense strand in whole or in part. In some embodiments, the shRNA is encoded in a vector; this vector may be the same or different from the vector encoding a CAR disclosed herein. In some embodiments, the vector encoding the shRNA comprises a promoter, a sense strand comprising a Tet2 target sequence, a loop, an anti-sense strand complementary to the sense strand in whole or in part, and, optionally, a polyT tail. In some embodiments, the promoter is a U6 promoter, optionally a human U6 promoter. Sequences of and methods of using U6 promoters for shRNA are known in the art and are found, for example, in Gao et al. Transcription, 8 (5): 275-287, 2017; Kunkel & Pederson, Genes & Dev 2:196-204, 1988; Goomer & Kunkel Nucleic Acid Res. 20:4903-4912, 1992; and Ma et al Molecular Therapy-Nucleic Acids 3: e161, 2014, all of which are incorporated by reference herein. Non-limiting examples of U6 promoter sequences (with the TATA box underlined) include:

(SEQ ID NO: 787)
TATATATCTTGTGGAAAGGACGAAACACC
(SEQ ID NO: 788)
TATATATCTTGTGGAAAGGACGAAACACCG
(SEQ ID NO: 789)
TTATATATCTTGTGGAAAGGACGAAACACC
(SEQ ID NO: 790)
TTATATATCTTGTGGAAAGGACGAAACACCA
(SEQ ID NO: 791)
TTATATATCTTGTGGAAAGGACGAAACACCG

In some embodiments, the sense strand comprising a Tet2 target sequence comprises 19, 20, or 21 nucleotides. In some embodiments, the anti-sense strand complementary to the sense strand in whole or in part comprises 19, 20, or 21 nucleotides, optionally, the same length as the sense strand. In some embodiments, the anti-sense strand complementary is complementary (when read from 3′ to 5′) to the sense strand comprising a Tet2 target sequence in part, for example, from, at least, positions 2 to 18 or from positions 1 to 9 and 11 to 19, 20, or 21. In some embodiments, the anti-sense strand complementary to the sense strand to the sense strand comprising a Tet2 target sequence is complementary (when read from 3′ to 5′) to the sense strand comprising a Tet2 target sequence in whole, i.e. over the entire length of the sense strand comprising a Tet2 target sequence, e.g., positions 1 to 19, 1 to 20, or 1 to 21. In some embodiments, the sense strand comprises one or more of the Tet2 target sequences provided in Table 4 of WO2017/049166, incorporated by reference herein. Non-limiting exemplary Tet2 target sequences include:

(SEQ ID NO: 418)
GGGTAAGCCAAGAAAGAAA
(SEQ ID NO: 419)
TTTCTTTCTTGGCTTACCC

The anti-sense strand, a reverse complement of SEQ ID NO: 418, is provided above as SEQ ID NO: 419, although it is noted that determination thereof can be readily performed based on skill in the art (for example, using the Sequence Manipulation Site available at bioinformations.org/sms2/rev_comp.html. In some embodiments, the loop is selected from sequences known in the art, such as but not limited to those disclosed in Bofill-De Ros & Gu, Methods 103:157-166, 2016; Gu et al. Cell 151 (4): 900-911, 2012; Cullen, Gene Ther. 13:503-508, 2006; and Schopman et al Antiviral Res. 86:204-211, 2014, all of which are incorporated by reference herein. Non-limiting exemplary loop sequences include:
GTTAATATTCATAGC (SEQ ID NO: 792)
CCTGACCCA (SEQ ID NO: 793)

In some embodiments, the vector encoding the shRNA comprises a polyT tail, i.e., 2, 3, 4, 5, 6, or more T nucleotides in sequence, for example: TT, TTT, TTTT, TTTTT, TTTTTT, TTTTTT, TTTTTTT, TTTTTTTTT, TTTTTTTTT etc. Non-limiting exemplary sequences comprising all the vector elements described in this paragraph include:

TABLE 29

| Promoter | Sense | Loop | Anti-sense | PolyT |
|---|---|---|---|---|
| TATATATCTTGTG | GGGTAAGCCAA | GTTAATA | TTTCTTTCTT | TTT |
| GAAAGGACGAAA | GAAAGAAA | TTCATAG | GGCTTACCC | TTT |
| CACCG (SEQ ID | (SEQ ID NO: 418) | C (SEQ ID | (SEQ ID NO: | |
| NO: 788) | | NO: 792) | 419) | |
| TATATATCTTGTG | GGGTAAGCCAA | CCTGACC | TTTCTTTCTT | TTT |
| GAAAGGACGAAA | GAAAGAAA | CA (SEQ | GGCTTACCC | TTT |
| CACCG (SEQ ID | (SEQ ID NO: 418) | ID NO: | (SEQ ID NO: | |
| NO: 788) | | 793) | 419) | |
| TTATATATCTTGT | GGGTAAGCCAA | GTTAATA | TTTCTTTCTT | TTT |
| GGAAAGGACGAA | GAAAGAAA | TTCATAG | GGCTTACCC | TTT |
| ACACCA (SEQ ID | (SEQ ID NO: 418) | C (SEQ ID | (SEQ ID NO: | |
| NO: 790) | | NO: 792) | 419) | |
| TTATATATCTTGT | GGGTAAGCCAA | CCTGACC | TTTCTTTCTT | TTT |
| GGAAAGGACGAA | GAAAGAAA | CA (SEQ | GGCTTACCC | TTT |
| ACACCA (SEQ ID | (SEQ ID NO: 418) | ID NO: | (SEQ ID NO: | |
| NO: 790) | | 793) | 419) | |
| TTATATATCTTGT | GGGTAAGCCAA | GTTAATA | TTTCTTTCTT | TTT |
| GGAAAGGACGAA | GAAAGAAA | TTCATAG | GGCTTACCC | TTT |
| ACACCG (SEQ ID | (SEQ ID NO: 418) | C (SEQ ID | (SEQ ID NO: | |
| NO: 791) | | NO: 792) | 419) | |
| TTATATATCTTGT | GGGTAAGCCAA | CCTGACC | TTTCTTTCTT | TTT |
| GGAAAGGACGAA | GAAAGAAA | CA (SEQ | GGCTTACCC | TTT |
| ACACCG (SEQ ID | (SEQ ID NO: 418) | ID NO: | (SEQ ID NO: | |
| NO: 791) | | 793) | 419) | |

It is appreciated that one of ordinary skill in the art is readily able to transcribe the DNA sequences disclosed herein, as pertaining to a vector encoding an shRNA, into RNA by replacing the “T” with “U”-thus, yielding the shRNA sequence. In some embodiments, the shRNA or the vector encoding the shRNA is used in step (ii) (i.e. the contacting step) of the method of making a population of cells (for example, T cells) that express a chimeric antigen receptor (CAR) disclosed herein above. Accordingly, step (ii) may further comprise contacting the population of cells (for example, T-cells) with shRNA, optionally the vector encoding the shRNA, described herein. In some embodiments, the vector encoding the shRNA further comprises a detectable tag, i.e. a tag that can be detected in cells contacted with the vector to indicate successful transduction. Non-limiting examples of such detectable tags are known, such as but not limited to fluorescent tags and/or artificial surface markers, optionally detectable by commercially available antibodies or other molecules. Further non-limiting examples include artificial surface markers such as truncated EGFR or biotin/streptavidin; such tags are well known in the art. In some embodiments, the vector encoding the shRNA comprises the nucleic acid encoding a CAR, disclosed herein above and used in step (ii) (i.e. the contacting step) of the method of making a population of cells (for example, T cells) that express a chimeric antigen receptor (CAR) disclosed herein above.

Co-Expression of CAR with a Chemokine Receptor

In embodiments, the CAR-expressing cell described herein, for example, CD19 CAR-expressing cell, further comprises a chemokine receptor molecule. Transgenic expression of chemokine receptors CCR2b or CXCR2 in T cells enhances trafficking to CCL2- or CXCL1-secreting solid tumors including melanoma and neuroblastoma (Craddock et al., J Immunother. 2010 October; 33 (8): 780-8 and Kershaw et al., Hum Gene Ther. 2002 Nov. 1; 13 (16): 1971-80). Thus, without wishing to be bound by theory, it is believed that chemokine receptors expressed in CAR-expressing cells that recognize chemokines secreted by tumors, for example, solid tumors, can improve homing of the CAR-expressing cell to the tumor, facilitate the infiltration of the CAR-expressing cell to the tumor, and enhances antitumor efficacy of the CAR-expressing cell. The chemokine receptor molecule can comprise a naturally occurring or recombinant chemokine receptor or a chemokine-binding fragment thereof. A chemokine receptor molecule suitable for expression in a CAR-expressing cell (for example, CAR-Tx) described herein include a CXC chemokine receptor (for example, CXCR1, CXCR2, CXCR3, CXCR4, CXCR5, CXCR6, or CXCR7), a CC chemokine receptor (for example, CCR1, CCR2, CCR3, CCR4, CCR5, CCR6, CCR7, CCR8, CCR9, CCR10, or CCR11), a CX3C chemokine receptor (for example, CX3CR1), a XC chemokine receptor (for example, XCR1), or a chemokine-binding fragment thereof. In some embodiments, the chemokine receptor molecule to be expressed with a CAR described herein is selected based on the chemokine(s) secreted by the tumor. In some embodiments, the CAR-expressing cell described herein further comprises, for example, expresses, a CCR2b receptor or a CXCR2 receptor. In some embodiments, the CAR described herein and the chemokine receptor molecule are on the same vector or are on two different vectors. In embodiments where the CAR described herein and the chemokine receptor molecule are on the same vector, the CAR and the chemokine receptor molecule are each under control of two different promoters or are under the control of the same promoter.

### Nucleic Acid Constructs Encoding a CAR

The present invention also provides an immune effector cell, for example, made by a method described herein, that includes a nucleic acid molecule encoding one or more CAR constructs described herein. In some embodiments, the nucleic acid molecule is provided as a messenger RNA transcript. In some embodiments, the nucleic acid molecule is provided as a DNA construct.

The nucleic acid molecules described herein can be a DNA molecule, an RNA molecule, or a combination thereof. In some embodiments, the nucleic acid molecule is an mRNA encoding a CAR polypeptide as described herein. In other embodiments, the nucleic acid molecule is a vector that includes any of the aforesaid nucleic acid molecules.

In some embodiments, the antigen binding domain of a CAR of the invention (for example, a scFv) is encoded by a nucleic acid molecule whose sequence has been codon optimized for expression in a mammalian cell. In some embodiments, entire CAR construct of the invention is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, for example, methods disclosed in at least U.S. Pat. Nos. 5,786,464 and 6,114,148.

Accordingly, in some embodiments, an immune effector cell, for example, made by a method described herein, includes a nucleic acid molecule encoding a chimeric antigen receptor (CAR), wherein the CAR comprises an antigen binding domain that binds to a tumor antigen described herein, a transmembrane domain (for example, a transmembrane domain described herein), and an intracellular signaling domain (for example, an intracellular signaling domain described herein) comprising a stimulatory domain, for example, a costimulatory signaling domain (for example, a costimulatory signaling domain described herein) and/or a primary signaling domain (for example, a primary signaling domain described herein, for example, a zeta chain described herein).

The present invention also provides vectors in which a nucleic acid molecule encoding a CAR, for example, a nucleic acid molecule described herein, is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, for example, a gammaretroviral vector. A gammaretroviral vector may include, for example, a promoter, a packaging signal (w), a primer binding site (PBS), one or more (for example, two) long terminal repeats (LTR), and a transgene of interest, for example, a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, for example, in Tobias Maetzig et al., “Gammaretroviral Vectors: Biology, Technology and Application” Viruses. 2011 June; 3 (6): 677-713.

In some embodiments, the vector comprising the nucleic acid encoding the desired CAR is an adenoviral vector (A5/35). In some embodiments, the expression of nucleic acids encoding CARs can be accomplished using of transposons such as sleeping beauty, crisper, CAS9, and zinc finger nucleases. See below June et al. 2009Nature Reviews Immunology 9.10:704-716, is incorporated herein by reference.

In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (for example, WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used.

Additional promoter elements, for example, enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Exemplary promoters include the CMV IE gene, EF-1α, ubiquitin C, or phosphoglycerokinase (PGK) promoters.

An example of a promoter that is capable of expressing a CAR encoding nucleic acid molecule in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving CAR expression from nucleic acid molecules cloned into a lentiviral vector. See, for example, Milone et al., Mol. Ther. 17 (8): 1453-1464 (2009). In some embodiments, the EF1a promoter comprises the sequence provided in the Examples.

Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1α promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Another example of a promoter is the phosphoglycerate kinase (PGK) promoter. In embodiments, a truncated PGK promoter (for example, a PGK promoter with one or more, for example, 1, 2, 5, 10, 100, 200, 300, or 400, nucleotide deletions when compared to the wild-type PGK promoter sequence) may be desired.

The nucleotide sequences of exemplary PGK promoters are provided below.

WT PGK Promoter:
(SEQ ID NO: 190)
ACCCCTCTCTCCAGCCACTAAGCCAGTTGCTCCCTCGGCTGACGGCTGC
ACGCGAGGCCTCCGAACGTCTTACGCCTTGTGGCGCGCCCGTCCTTGTC
CCGGGTGTGATGGCGGGGTGTGGGGCGGAGGGCGTGGCGGGGAAGGGCC
GGCGACGAGAGCCGCGCGGGACGACTCGTCGGCGATAACCGGTGTCGGG
TAGCGCCAGCCGCGCGACGGTAACGAGGGACCGCGACAGGCAGACGCTC
CCATGATCACTCTGCACGCCGAAGGCAAATAGTGCAGGCCGTGCGGCGC
TTGGCGTTCCTTGGAAGGGCTGAATCCCCGCCTCGTCCTTCGCAGCGGC
CCCCCGGGTGTTCCCATCGCCGCTTCTAGGCCCACTGCGACGCTTGCCT
GCACTTCTTACACGCTCTGGGTCCCAGCCGCGGCGACGCAAAGGGCCTT
GGTGCGGGTCTCGTCGGCGCAGGGACGCGTTTGGGTCCCGACGGAACCT
TTTCCGCGTTGGGGTTGGGGCACCATAAGCT
Exemplary truncated PGK Promoters:
PGK100:
(SEQ ID NO: 198)
ACCCCTCTCTCCAGCCACTAAGCCAGTTGCTCCCTCGGCTGACGGCTGC
ACGCGAGGCCTCCGAACGTCTTACGCCTTGTGGCGCGCCCGTCCTTGTC
CCGGGTGTGATGGCGGGGTG
PGK200:
(SEQ ID NO: 191)
ACCCCTCTCTCCAGCCACTAAGCCAGTTGCTCCCTCGGCTGACGGCTGC
ACGCGAGGCCTCCGAACGTCTTACGCCTTGTGGCGCGCCCGTCCTTGTC
CCGGGTGTGATGGCGGGGTGTGGGGCGGAGGGCGTGGCGGGGAAGGGCC
GGCGACGAGAGCCGCGCGGGACGACTCGTCGGCGATAACCGGTGTCGGG
TAGCGCCAGCCGCGCGACGGTAACG
PGK300:
(SEQ ID NO: 192)
ACCCCTCTCTCCAGCCACTAAGCCAGTTGCTCCCTCGGCTGACGGCTGC
ACGCGAGGCCTCCGAACGTCTTACGCCTTGTGGCGCGCCCGTCCTTGTC
CCGGGTGTGATGGCGGGGTGTGGGGCGGAGGGCGTGGCGGGGAAGGGCC
GGCGACGAGAGCCGCGCGGGACGACTCGTCGGCGATAACCGGTGTCGGG
TAGCGCCAGCCGCGCGACGGTAACGAGGGACCGCGACAGGCAGACGCTC
CCATGATCACTCTGCACGCCGAAGGCAAATAGTGCAGGCCGTGCGGCGC
TTGGCGTTCCTTGGAAGGGCTGAATCCCCG
PGK400:
(SEQ ID NO: 193)
ACCCCTCTCTCCAGCCACTAAGCCAGTTGCTCCCTCGGCTGACGGCTGC
ACGCGAGGCCTCCGAACGTCTTACGCCTTGTGGCGCGCCCGTCCTTGTC
TGGCGGGGTGTGGGGCGGAGGGCGTGGCGGGGAAGGGCCGGCGACGAGA
GCCGGGTGTGACCGCGCGGGACGACTCGTCGGCGATAACCGGTGTCGGG
TAGCGCCAGCCGCGCGACGGTAACGAGGGACCGCGACAGGCAGACGCTC
CCATGATCACTCTGCACGCCGAAGGCAAATAGTGCAGGCCGTGCGGCGC
TTGGCGTTCCTTGGAAGGGCTGAATCCCCGCCTCGTCCTTCGCAGCGGC
CCCCCGGGTGTTCCCATCGCCGCTTCTAGGCCCACTGCGACGCTTGCCT
GCACTTCTTACACGCTCTGGGTCCCAGCCG

A vector may also include, for example, a signal sequence to facilitate secretion, a polyadenylation signal and transcription terminator (for example, from Bovine Growth Hormone (BGH) gene), an element allowing episomal replication and replication in prokaryotes (for example SV40 origin and ColE1 or others known in the art) and/or elements to allow selection (for example, ampicillin resistance gene and/or zeocin marker).

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, for example, enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (for example, Ui-Tei et al., 2000 FEBS Letters 479:79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In embodiments, the vector may comprise two or more nucleic acid sequences encoding a CAR, for example, a CAR described herein, for example, a CD19 CAR, and a second CAR, for example, an inhibitory CAR or a CAR that specifically binds to an antigen other than CD19. In such embodiments, the two or more nucleic acid sequences encoding the CAR are encoded by a single nucleic molecule in the same frame and as a single polypeptide chain. In some embodiments, the two or more CARs, can, for example, be separated by one or more peptide cleavage sites. (for example, an auto-cleavage site or a substrate for an intracellular protease). Examples of peptide cleavage sites include T2A, P2A, E2A, or F2A sites.

Methods of introducing and expressing genes into a cell are known in the art. In the context of an expression vector, the vector can be readily introduced into a host cell, for example, mammalian, bacterial, yeast, or insect cell by any method, for example, one known in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). A suitable method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, for example, human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (for example, an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In some embodiments, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL.). Stock solutions of lipids in chloroform or chloroform/methanol can be stored at about-20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5:505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant nucleic acid sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, for example, by immunological means (ELISAs and Western blots) or by assays described herein to identify agents falling within the scope of the invention.

### Natural Killer Cell Receptor (NKR) CARs

In some embodiments, the CAR molecule described herein comprises one or more components of a natural killer cell receptor (NKR), thereby forming an NKR-CAR. The NKR component can be a transmembrane domain, a hinge domain, or a cytoplasmic domain from any of the following natural killer cell receptors: killer cell immunoglobulin-like receptor (KIR), for example, KIR2DL1, KIR2DL2/L3, KIR2DL4, KIR2DL5A, KIR2DL5B, KIR2DS1, KIR2DS2, KIR2DS3, KIR2DS4, DIR2DS5, KIR3DL1/S1, KIR3DL2, KIR3DL3, KIR2DP1, and KIR3DP1; natural cytotoxicity receptor (NCR), for example, NKp30, NKp44, NKp46; signaling lymphocyte activation molecule (SLAM) family of immune cell receptors, for example, CD48, CD229, 2B4, CD84, NTB-A, CRACC, BLAME, and CD2F-10; Fc receptor (FcR), for example, CD16, and CD64; and Ly49 receptors, for example, LY49A, LY49C. The NKR-CAR molecules described herein may interact with an adaptor molecule or intracellular signaling domain, for example, DAP12. Exemplary configurations and sequences of CAR molecules comprising NKR components are described in International Publication No. WO2014/145252, the contents of which are hereby incorporated by reference.

### Split CAR

In some embodiments, the CAR-expressing cell uses a split CAR. The split CAR approach is described in more detail in publications WO2014/055442 and WO2014/055657. Briefly, a split CAR system comprises a cell expressing a first CAR having a first antigen binding domain and a costimulatory domain (for example, 41BB), and the cell also expresses a second CAR having a second antigen binding domain and an intracellular signaling domain (for example, CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens.

### Strategies for Regulating Chimeric Antigen Receptors

In some embodiments, a regulatable CAR (RCAR) where the CAR activity can be controlled is desirable to optimize the safety and efficacy of a CAR therapy. There are many ways CAR activities can be regulated. For example, inducible apoptosis using, for example, a caspase fused to a dimerization domain (see, for example, Di Stasa et al., N Engl. J. Med. 2011 Nov. 3; 365 (18): 1673-1683), can be used as a safety switch in the CAR therapy of the instant invention. In some embodiments, the cells (for example, T cells or NK cells) expressing a CAR of the present invention further comprise an inducible apoptosis switch, wherein a human caspase (for example, caspase 9) or a modified version is fused to a modification of the human FKB protein that allows conditional dimerization. In the presence of a small molecule, such as a rapalog (for example, AP 1903, AP20187), the inducible caspase (for example, caspase 9) is activated and leads to the rapid apoptosis and death of the cells (for example, T cells or NK cells) expressing a CAR of the present invention. Examples of a caspase-based inducible apoptosis switch (or one or more aspects of such a switch) have been described in, for example, US2004040047; US20110286980; US20140255360; WO1997031899; WO2014151960; WO2014164348; WO2014197638; WO2014197638; all of which are incorporated by reference herein.

In another example, CAR-expressing cells can also express an inducible Caspase-9 (iCaspase-9) molecule that, upon administration of a dimerizer drug (for example, rimiducid (also called AP1903 (Bellicum Pharmaceuticals) or AP20187 (Ariad)) leads to activation of the Caspase-9 and apoptosis of the cells. The iCaspase-9 molecule contains a chemical inducer of dimerization (CID) binding domain that mediates dimerization in the presence of a CID. This results in inducible and selective depletion of CAR-expressing cells. In some cases, the iCaspase-9 molecule is encoded by a nucleic acid molecule separate from the CAR-encoding vector(s). In some cases, the iCaspase-9 molecule is encoded by the same nucleic acid molecule as the CAR-encoding vector. The iCaspase-9 can provide a safety switch to avoid any toxicity of CAR-expressing cells. See, for example, Song et al. Cancer Gene Ther. 2008; 15 (10): 667-75; Clinical Trial Id. No. NCT02107963; and Di Stasi et al. N. Engl. J. Med. 2011; 365:1673-83.

Alternative strategies for regulating the CAR therapy of the instant invention include utilizing small molecules or antibodies that deactivate or turn off CAR activity, for example, by deleting CAR-expressing cells, for example, by inducing antibody dependent cell-mediated cytotoxicity (ADCC). For example, CAR-expressing cells described herein may also express an antigen that is recognized by molecules capable of inducing cell death, for example, ADCC or complement-induced cell death. For example, CAR expressing cells described herein may also express a receptor capable of being targeted by an antibody or antibody fragment. Examples of such receptors include EpCAM, VEGFR, integrins (for example, integrins αvβ3, α4, αI¾β3, α4β7, α5β1, ανβ3, αν), members of the TNF receptor superfamily (for example, TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/lgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (for example, versions preserving one or more extracellular epitopes but lacking one or more regions within the cytoplasmic domain).

For example, a CAR-expressing cell described herein may also express a truncated epidermal growth factor receptor (EGFR) which lacks signaling capacity but retains the epitope that is recognized by molecules capable of inducing ADCC, for example, cetuximab (ERBITUX®), such that administration of cetuximab induces ADCC and subsequent depletion of the CAR-expressing cells (see, for example, WO2011/056894, and Jonnalagadda et al., Gene Ther. 2013; 20 (8) 853-860). Another strategy includes expressing a highly compact marker/suicide gene that combines target epitopes from both CD32 and CD20 antigens in the CAR-expressing cells described herein, which binds rituximab, resulting in selective depletion of the CAR-expressing cells, for example, by ADCC (see, for example, Philip et al., Blood. 2014; 124 (8) 1277-1287). Other methods for depleting CAR-expressing cells described herein include administration of CAMPATH, a monoclonal anti-CD52 antibody that selectively binds and targets mature lymphocytes, for example, CAR-expressing cells, for destruction, for example, by inducing ADCC. In other embodiments, the CAR-expressing cell can be selectively targeted using a CAR ligand, for example, an anti-idiotypic antibody. In some embodiments, the anti-idiotypic antibody can cause effector cell activity, for example, ADCC or ADC activities, thereby reducing the number of CAR-expressing cells. In other embodiments, the CAR ligand, for example, the anti-idiotypic antibody, can be coupled to an agent that induces cell killing, for example, a toxin, thereby reducing the number of CAR-expressing cells. Alternatively, the CAR molecules themselves can be configured such that the activity can be regulated, for example, turned on and off, as described below.

In other embodiments, a CAR-expressing cell described herein may also express a target protein recognized by the T cell depleting agent. In some embodiments, the target protein is CD20 and the T cell depleting agent is an anti-CD20 antibody, for example, rituximab. In some embodiments, the T cell depleting agent is administered once it is desirable to reduce or eliminate the CAR-expressing cell, for example, to mitigate the CAR induced toxicity. In other embodiments, the T cell depleting agent is an anti-CD52 antibody, for example, alemtuzumab, as described in the Examples herein.

In other embodiments, an RCAR comprises a set of polypeptides, typically two in the simplest embodiments, in which the components of a standard CAR described herein, for example, an antigen binding domain and an intracellular signaling domain, are partitioned on separate polypeptides or members. In some embodiments, the set of polypeptides include a dimerization switch that, upon the presence of a dimerization molecule, can couple the polypeptides to one another, for example, can couple an antigen binding domain to an intracellular signaling domain. In some embodiments, a CAR of the present invention utilizes a dimerization switch as those described in, for example, WO2014127261, which is incorporated by reference herein. Additional description and exemplary configurations of such regulatable CARs are provided herein and in, for example, paragraphs 527-551 of International Publication No. WO 2015/090229 filed Mar. 13, 2015, which is incorporated by reference in its entirety. In some embodiments, an RCAR involves a switch domain, for example, a FKBP switch domain, as set out SEQ ID NO: 275, or comprise a fragment of FKBP having the ability to bind with FRB, for example, as set out in SEQ ID NO: 276. In some embodiments, the RCAR involves a switch domain comprising a FRB sequence, for example, as set out in SEQ ID NO: 277, or a mutant FRB sequence, for example, as set out in any of SEQ ID NOs. 278-283.

(SEQ ID NO: 275)
DVPDYASLGGPSSPKKKRKVSRGVQVETISPGDGRTFPKRGQTCVVHYT
GMLEDGKKFDSSRDRNKPFKFMLGKQEVIRGWEEGVAQMSVGQRAKLTI
SPDYAYGATGHPGIIPPHATLVFDVELLKLETSY
(SEQ ID NO: 276)
VQVETISPGDGRTFPKRGQTCVVHYTGMLEDGKKFDSSRDRNKPFKFML
GKQEVIRGWEEGVAQMSVGQRAKLTISPDYAYGATGHPGIIPPHATLVF
DVELLKLETS
(SEQ ID NO: 277)
ILWHEMWHEGLEEASRLYFGERNVKGMFEVLEPLHAMMERGPQTLKETS
FNQAYGRDLMEAQEWCRKYMKSGNVKDLTQAWDLYYHVFRRISK

TABLE 18
Exemplary mutant FRB having increased affinity for a dimerization molecule.

| | | SEQ |
|---|---|---|
| | | ID |
| FRB mutant | Amino Acid Sequence | NO: |
| E2032I mutant | ILWHEMWHEGLIEASRLYFGERNVKGMFEVLEPLHAMMERGPQTLKETSFNQAYG | 278 |
| | RDLMEAQEWCRKYMKSGNVKDLTQAWDLYYHVFRRISKTS | |
| E2032L mutant | ILWHEMWHEGLLEASRLYFGERNVKGMFEVLEPLHAMMERGPQTLKETSFNQAYG | 279 |
| | RDLMEAQEWCRKYMKSGNVKDLTQAWDLYYHVFRRISKTS | |
| T2098L mutant | ILWHEMWHEGLEEASRLYFGERNVKGMFEVLEPLHAMMERGPQTLKETSFNQAYG | 280 |
| | RDLMEAQEWCRKYMKSGNVKDLLQAWDLYYHVFRRISKTS | |
| E2032, T2098 | ILWHEMWHEGLXEASRLYFGERNVKGMFEVLEPLHAMMERGPQTLKETSFNQAYG | 281 |
| mutant | RDLMEAQEWCRKYMKSGNVKDLXQAWDLYYHVFRRISKTS | |
| E2032I, T2098L | ILWHEMWHEGLIEASRLYFGERNVKGMFEVLEPLHAMMERGPQTLKETSFNQAYG | 282 |
| mutant | RDLMEAQEWCRKYMKSGNVKDLLQAWDLYYHVFRRISKTS | |
| E2032L, T2098L | ILWHEMWHEGLLEASRLYFGERNVKGMFEVLEPLHAMMERGPQTLKETSFNQAYG | 283 |
| mutant | RDLMEAQEWCRKYMKSGNVKDLLQAWDLYYHVFRRISKTS | |

### RNA Transfection

Disclosed herein are methods for producing an in vitro transcribed RNA CAR. RNA CAR and methods of using the same are described, for example, in paragraphs 553-570 of in International Application WO2015/142675, filed Mar. 13, 2015, which is herein incorporated by reference in its entirety.

An immune effector cell can include a CAR encoded by a messenger RNA (mRNA). In some embodiments, the mRNA encoding a CAR described herein is introduced into an immune effector cell, for example, made by a method described herein, for production of a CAR-expressing cell.

In some embodiments, the in vitro transcribed RNA CAR can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired temple for in vitro transcription is a CAR described herein. For example, the template for the RNA CAR comprises an extracellular region comprising a single chain variable domain of an antibody to a tumor associated antigen described herein; a hinge region (for example, a hinge region described herein), a transmembrane domain (for example, a transmembrane domain described herein such as a transmembrane domain of CD8a); and a cytoplasmic region that includes an intracellular signaling domain, for example, an intracellular signaling domain described herein, for example, comprising the signaling domain of CD3-zeta and the signaling domain of 4-1BB.

In some embodiments, the DNA to be used for PCR contains an open reading frame. The DNA can be from a naturally occurring DNA sequence from the genome of an organism. In some embodiments, the nucleic acid can include some or all of the 5′ and/or 3′ untranslated regions (UTRs). The nucleic acid can include exons and introns. In some embodiments, the DNA to be used for PCR is a human nucleic acid sequence. In some embodiments, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In some embodiments, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA in embodiments has 5′ and 3′ UTRs. In some embodiments, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In some embodiments, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be 5′UTR of an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In some embodiments, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In some embodiments, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of poly(A)/T stretches into a DNA template is molecular cloning. However, poly(A)/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with poly(A)/T 3′ stretch without cloning highly desirable.

The poly(A)/T segment of the transcriptional DNA template can be produced during PCR by using a reverse primer containing a polyT tail, such as 100T tail (SEQ ID NO: 31) (size can be 50-5000 T (SEQ ID NO: 32)), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In some embodiments, the poly(A) tail is between 100 and 5000 adenosines (for example, SEQ ID NO: 33).

Poly(A) tails of RNAs can be further extended following in vitro transcription with the use of a poly(A) polymerase, such as E. coli poly(A) polymerase (E-PAP). In some embodiments, increasing the length of a poly(A) tail from 100 nucleotides to between 300 and 400 nucleotides (SEQ ID NO: 34) results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. In some embodiments, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12 (8): 861-70 (2001).

### Non-Viral Delivery Methods

In some embodiments, non-viral methods can be used to deliver a nucleic acid encoding a CAR described herein into a cell or tissue or a subject.

In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition.

Exemplary methods of nucleic acid delivery using a transposon include a Sleeping Beauty transposon system (SBTS) and a piggyBac™ (PB) transposon system. See, for example, Aronovich et al. Hum. Mol. Genet. 20.R1 (2011): R14-20; Singh et al. Cancer Res. 15 (2008): 2961-2971; Huang et al. Mol. Ther. 16 (2008): 580-589; Grabundzija et al. Mol. Ther. 18 (2010): 1200-1209; Kebriaei et al. Blood. 122.21 (2013): 166; Williams. Molecular Therapy 16.9 (2008): 1515-16; Bell et al. Nat. Protoc. 2.12 (2007): 3153-65; and Ding et al. Cell. 122.3 (2005): 473-83, all of which are incorporated herein by reference.

The SBTS includes two components: 1) a transposon containing a transgene and 2) a source of transposase enzyme. The transposase can transpose the transposon from a carrier plasmid (or other donor DNA) to a target DNA, such as a host cell chromosome/genome. For example, the transposase binds to the carrier plasmid/donor DNA, cuts the transposon (including transgene(s)) out of the plasmid, and inserts it into the genome of the host cell. See, for example, Aronovich et al. supra.

Exemplary transposons include a pT2-based transposon. See, for example, Grabundzija et al. Nucleic Acids Res. 41.3 (2013): 1829-47; and Singh et al. Cancer Res. 68.8 (2008): 2961-2971, all of which are incorporated herein by reference. Exemplary transposases include a Tcl/mariner-type transposase, for example, the SB10 transposase or the SB11 transposase (a hyperactive transposase which can be expressed, for example, from a cytomegalovirus promoter). See, for example, Aronovich et al.; Kebriaei et al.; and Grabundzija et al., all of which are incorporated herein by reference.

Use of the SBTS permits efficient integration and expression of a transgene, for example, a nucleic acid encoding a CAR described herein. Provided herein are methods of generating a cell, for example, T cell or NK cell, that stably expresses a CAR described herein, for example, using a transposon system such as SBTS.

In accordance with methods described herein, in some embodiments, one or more nucleic acids, for example, plasmids, containing the SBTS components are delivered to a cell (for example, T or NK cell). For example, the nucleic acid(s) are delivered by standard methods of nucleic acid (for example, plasmid DNA) delivery, for example, methods described herein, for example, electroporation, transfection, or lipofection. In some embodiments, the nucleic acid contains a transposon comprising a transgene, for example, a nucleic acid encoding a CAR described herein. In some embodiments, the nucleic acid contains a transposon comprising a transgene (for example, a nucleic acid encoding a CAR described herein) as well as a nucleic acid sequence encoding a transposase enzyme. In other embodiments, a system with two nucleic acids is provided, for example, a dual-plasmid system, for example, where a first plasmid contains a transposon comprising a transgene, and a second plasmid contains a nucleic acid sequence encoding a transposase enzyme. For example, the first and the second nucleic acids are co-delivered into a host cell.

In some embodiments, cells, for example, T or NK cells, are generated that express a CAR described herein by using a combination of gene insertion using the SBTS and genetic editing using a nuclease (for example, Zinc finger nucleases (ZFNs), Transcription Activator-Like Effector Nucleases (TALENs), the CRISPR/Cas system, or engineered meganuclease re-engineered homing endonucleases).

In some embodiments, use of a non-viral method of delivery permits reprogramming of cells, for example, T or NK cells, and direct infusion of the cells into a subject. Advantages of non-viral vectors include but are not limited to the ease and relatively low cost of producing sufficient amounts required to meet a patient population, stability during storage, and lack of immunogenicity.

### Methods of Manufacture/Production

In some embodiments, the methods disclosed herein further include administering a T cell depleting agent after treatment with the cell (for example, an immune effector cell as described herein), thereby reducing (for example, depleting) the CAR-expressing cells (for example, the CD19CAR-expressing cells). Such T cell depleting agents can be used to effectively deplete CAR-expressing cells (for example, CD19CAR-expressing cells) to mitigate toxicity. In some embodiments, the CAR-expressing cells were manufactured according to a method herein, for example, assayed (for example, before or after transfection or transduction) according to a method herein.

In some embodiments, the T cell depleting agent is administered one, two, three, four, or five weeks after administration of the cell, for example, the population of immune effector cells, described herein.

In some embodiments, the T cell depleting agent is an agent that depletes CAR-expressing cells, for example, by inducing antibody dependent cell-mediated cytotoxicity (ADCC) and/or complement-induced cell death. For example, CAR-expressing cells described herein may also express an antigen (for example, a target antigen) that is recognized by molecules capable of inducing cell death, for example, ADCC or complement-induced cell death. For example, CAR expressing cells described herein may also express a target protein (for example, a receptor) capable of being targeted by an antibody or antibody fragment. Examples of such target proteins include, but are not limited to, EpCAM, VEGFR, integrins (for example, integrins ανβ3, α4, αI¾β3, α4β7, α5β1, ανβ3, αν), members of the TNF receptor superfamily (for example, TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/lgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (for example, versions preserving one or more extracellular epitopes but lacking one or more regions within the cytoplasmic domain).

In some embodiments, the CAR expressing cell co-expresses the CAR and the target protein, for example, naturally expresses the target protein or is engineered to express the target protein. For example, the cell, for example, the population of immune effector cells, can include a nucleic acid (for example, vector) comprising the CAR nucleic acid (for example, a CAR nucleic acid as described herein) and a nucleic acid encoding the target protein.

In some embodiments, the T cell depleting agent is a CD52 inhibitor, for example, an anti-CD52 antibody molecule, for example, alemtuzumab.

In other embodiments, the cell, for example, the population of immune effector cells, expresses a CAR molecule as described herein (for example, CD19CAR) and the target protein recognized by the T cell depleting agent. In some embodiments, the target protein is CD20. In embodiments where the target protein is CD20, the T cell depleting agent is an anti-CD20 antibody, for example, rituximab.

In further embodiments of any of the aforesaid methods, the methods further include transplanting a cell, for example, a hematopoietic stem cell, or a bone marrow, into the mammal.

In some embodiments, the invention features a method of conditioning a mammal prior to cell transplantation. The method includes administering to the mammal an effective amount of the cell comprising a CAR nucleic acid or polypeptide, for example, a CD19 CAR nucleic acid or polypeptide. In some embodiments, the cell transplantation is a stem cell transplantation, for example, a hematopoietic stem cell transplantation, or a bone marrow transplantation. In other embodiments, conditioning a subject prior to cell transplantation includes reducing the number of target-expressing cells in a subject, for example, CD19-expressing normal cells or CD19-expressing cancer cells.

### Elutriation

In some embodiments, the methods described herein feature an elutriation method that removes unwanted cells, for example, monocytes and blasts, thereby resulting in an improved enrichment of desired immune effector cells suitable for CAR expression. In some embodiments, the elutriation method described herein is optimized for the enrichment of desired immune effector cells suitable for CAR expression from a previously frozen sample, for example, a thawed sample. In some embodiments, the elutriation method described herein provides a preparation of cells with improved purity as compared to a preparation of cells collected from the elutriation protocols known in the art. In some embodiments, the elutriation method described herein includes using an optimized viscosity of the starting sample, for example, cell sample, for example, thawed cell sample, by dilution with certain isotonic solutions (for example, PBS), and using an optimized combination of flow rates and collection volume for each fraction collected by an elutriation device. Exemplary elutriation methods that could be applied in the present invention are described on pages 48-51 of WO 2017/117112, herein incorporated by reference in its entirety.

### Density Gradient Centrifugation

Manufacturing of adoptive cell therapeutic product requires processing the desired cells, for example, immune effector cells, away from a complex mixture of blood cells and blood elements present in peripheral blood apheresis starting materials. Peripheral blood-derived lymphocyte samples have been successfully isolated using density gradient centrifugation through Ficoll solution. However, Ficoll is not a preferred reagent for isolating cells for therapeutic use, as Ficoll is not qualified for clinical use. In addition, Ficoll contains glycol, which has toxic potential to the cells. Furthermore, Ficoll density gradient centrifugation of thawed apheresis products after cryopreservation yields a suboptimal T cell product, for example, as described in the Examples herein. For example, a loss of T cells in the final product, with a relative gain of non-T cells, especially undesirable B cells, blast cells and monocytes was observed in cell preparations isolated by density gradient centrifugation through Ficoll solution.

Without wishing to be bound by theory, it is believed that immune effector cells, for example, T cells, dehydrate during cryopreservation to become denser than fresh cells. Without wishing to be bound by theory, it is also believed that immune effector cells, for example, T cells, remain denser longer than the other blood cells, and thus are more readily lost during Ficoll density gradient separation as compared to other cells. Accordingly, without wishing to be bound by theory, a medium with a density greater than Ficoll is believed to provide improved isolation of desired immune effector cells in comparison to Ficoll or other mediums with the same density as Ficoll, for example, 1.077 g/mL.

In some embodiments, the density gradient centrifugation method described herein includes the use of a density gradient medium comprising iodixanol. In some embodiments, the density gradient medium comprises about 60% iodixanol in water.

In some embodiments, the density gradient centrifugation method described herein includes the use of a density gradient medium having a density greater than Ficoll. In some embodiments, the density gradient centrifugation method described herein includes the use of a density gradient medium having a density greater than 1.077 g/mL, for example, greater than 1.077 g/mL, greater than 1.1 g/mL, greater than 1.15 g/mL, greater than 1.2 g/mL, greater than 1.25 g/mL, greater than 1.3 g/mL, greater than 1.31 g/mL. In some embodiments, the density gradient medium has a density of about 1.32 g/mL.

Additional embodiments of density gradient centrifugation are described on pages 51-53 of WO 2017/117112, herein incorporated by reference in its entirety.

### Enrichment by Selection

Provided herein are methods for selection of specific cells to improve the enrichment of the desired immune effector cells suitable for CAR expression. In some embodiments, the selection comprises a positive selection, for example, selection for the desired immune effector cells. In some embodiments, the selection comprises a negative selection, for example, selection for unwanted cells, for example, removal of unwanted cells. In embodiments, the positive or negative selection methods described herein are performed under flow conditions, for example, by using a flow-through device, for example, a flow-through device described herein. Exemplary positive and negative selections are described on pages 53-57 of WO 2017/117112, herein incorporated by reference in its entirety. Selection methods can be performed under flow conditions, for example, by using a flow-through device, also referred to as a cell processing system, to further enrich a preparation of cells for desired immune effector cells, for example, T cells, suitable for CAR expression. Exemplary flow-through devices are described on pages 57-70 of WO 2017/117112, herein incorporated by reference in its entirety. Exemplary cell separation and debeading methods are described on pages 70-78 of WO 2017/117112, herein incorporated by reference in its entirety.

Selection procedures are not limited to ones described on pages 57-70 of WO 2017/117112. Negative T cell selection via removal of unwanted cells with CD19, CD14 and CD26 Miltenyi beads in combination with column technology (CliniMACS® Plus or CliniMACS® Prodigy®) or positive T cell selection with a combination of CD4 and CD8 Miltenyi beads and column technology (CliniMACS® Plus or CliniMACS® Prodigy®) can be used. Alternatively, column-free technology with releasable CD3 beads (GE Healthcare) can be used.

In addition, bead-free technologies such as ThermoGenesis X-series devices can be utilized as well.

### Clinical Applications

All of the processes herein may be conducted according to clinical good manufacturing practice (cGMP) standards.

The processes may be used for cell purification, enrichment, harvesting, washing, concentration or for cell media exchange, particularly during the collection of raw, starting materials (particularly cells) at the start of the manufacturing process, as well as during the manufacturing process for the selection or expansion of cells for cell therapy.

The cells may include any plurality of cells. The cells may be of the same cell type, or mixed cell types. In addition, the cells may be from one donor, such as an autologous donor or a single allogenic donor for cell therapy. The cells may be obtained from patients by, for example, leukapheresis or apheresis. The cells may include T cells, for example may include a population that has greater than 50% T cells, greater than 60% T cells, greater than 70% T cells, greater than 80% T cells, or 90% T cells.

Selection processes may be particularly useful in selecting cells prior to culture and expansion. For instance, paramagnetic particles coated with anti-CD3 and/or anti CD28 may be used to select T cells for expansion or for introduction of a nucleic acid encoding a chimeric antigen receptor (CAR) or other protein. Such a process is used to produce CTL019 T cells for treatment of acute lymphoblastic leukemia (ALL).

The debeading processes and modules disclosed herein may be particularly useful in the manufacture of cells for cell therapy, for example in purifying cells prior to, or after, culture and expansion. For instance, paramagnetic particles coated with anti-CD3 and/or anti CD28 antibodies may be used to selectively expand T cells, for example T cells that are, or will be, modified by introduction of a nucleic acid encoding a chimeric antigen receptor (CAR) or other protein, such that the CAR is expressed by the T cells. During the manufacture of such T cells, the debeading processes or modules may be used to separate T cells from the paramagnetic particles. Such a debeading process or module is used to produce, for example, CTL019 T cells for treatment of acute lymphoblastic leukemia (ALL).

In one such process, illustrated here by way of example, cells, for example, T cells, are collected from a donor (for example, a patient to be treated with an autologous chimeric antigen receptor T cell product) via apheresis (for example, leukapheresis). Collected cells may then be optionally purified, for example, by an elutriation step, or via positive or negative selection of target cells (for example, T cells). Paramagnetic particles, for example, anti-CD3/anti-CD28-coated paramagnetic particles, may then be added to the cell population, to expand the T cells. The process may also include a transduction step, wherein nucleic acid encoding one or more desired proteins, for example, a CAR, for example a CAR targeting CD19, is introduced into the cell. The nucleic acid may be introduced in a lentiviral vector. The cells, for example, the lentivirally transduced cells, may then be expanded for a period of days, for example, 1, 2, 3, 4, 5, 6, 7, 8, 9, 10, or more days, for example in the presence of a suitable medium. After expansion, the debeading processes/modules disclosed herein may be used to separate the desired T cells from the paramagnetic particles. The process may include one or more debeading steps according to the processes of the present disclosure. The debeaded cells may then be formulated for administration to the patient. Examples of CAR T cells and their manufacture are further described, for example, in WO2012/079000, which is incorporated herein by reference in its entirety. The systems and methods of the present disclosure may be used for any cell separation/purification/debeading processes described in or associated with WO2012/079000. Additional CAR T manufacturing processes are described in, for example, WO2016109410 and WO2017117112, herein incorporated by reference in their entireties.

The systems and methods herein may similarly benefit other cell therapy products by wasting fewer desirable cells, causing less cell trauma, and more reliably removing magnetic and any non-paramagnetic particles from cells with less or no exposure to chemical agents, as compared to conventional systems and methods.

Although only exemplary embodiments of the disclosure are specifically described above, it will be appreciated that modifications and variations of these examples are possible without departing from the spirit and intended scope of the disclosure. For example, the magnetic modules and systems containing them may be arranged and used in a variety of configurations in addition to those described. Besides, non-magnetic modules can be utilized as well. In addition, the systems and methods may include additional components and steps not specifically described herein. For instance, methods may include priming, where a fluid is first introduced into a component to remove bubbles and reduce resistance to cell suspension or buffer movement. Furthermore, embodiments may include only a portion of the systems described herein for use with the methods described herein. For example, embodiments may relate to disposable modules, hoses, etc. usable within non-disposable equipment to form a complete system able to separate or debead cells to produce a cell product.

Additional manufacturing methods and processes that can be combined with the present invention have been described in the art. For examples, pages 86-91 of WO 2017/117112 describe improved wash steps and improved manufacturing process.

### Sources of Immune Effector Cells

This section provides additional methods or steps for obtaining an input sample comprising desired immune effector cells, isolating and processing desired immune effector cells, for example, T cells, and removing unwanted materials, for example, unwanted cells. The additional methods or steps described in this section can be used in combination with any of the elutriation, density gradient centrifugation, selection under flow conditions, or improved wash step described in the preceding sections.

A source of cells, for example, T cells or natural killer (NK) cells, can be obtained from a subject. Examples of subjects include humans, monkeys, chimpanzees, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In some embodiments of the present disclosure, immune effector cells, for example, T cells, can be obtained from a unit of blood collected from a subject using any number of techniques known to the skilled artisan, and any of the methods disclosed herein, in any combination of steps thereof. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and, optionally, to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. In some embodiments, the cells are washed using the improved wash step described herein.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate™, or the Haemonetics Cell Saver 5), Haemonetics Cell Saver Elite (GE Healthcare Sepax or Sefia), or a device utilizing the spinning membrane filtration technology (Fresenius Kabi LOVO), according to the manufacturer's instructions. After washing, the cells may be resuspended in a variety of biocompatible buffers, such as, for example, Ca-free, Mg-free PBS, PlasmaLyte A, PBS-EDTA supplemented with human serum albumin (HSA), or other saline solution with or without buffer. Alternatively, the undesirable components of the apheresis sample may be removed and the cells directly resuspended in culture media.

In some embodiments, desired immune effector cells, for example, T cells, are isolated from peripheral blood lymphocytes by lysing the red blood cells and depleting the monocytes, for example, by centrifugation through a PERCOLL™ gradient or by counterflow centrifugal elutriation.

The methods described herein can include, for example, selection of a specific subpopulation of immune effector cells, for example, T cells, that are a T regulatory cell-depleted population, for example, CD25+ depleted cells or CD25^{high} depleted cells, using, for example, a negative selection technique, for example, described herein. In some embodiments, the population of T regulatory-depleted cells contains less than 30%, 25%, 20%, 15%, 10%, 5%, 4%, 3%, 2%, 1% of CD25+ cells or CD25^{high} cells.

In some embodiments, T regulatory cells, for example, CD25+ T cells or CD25^{high} T cells, are removed from the population using an anti-CD25 antibody, or fragment thereof, or a CD25-binding ligand, for example IL-2. In some embodiments, the anti-CD25 antibody, or fragment thereof, or CD25-binding ligand is conjugated to a substrate, for example, a bead, or is otherwise coated on a substrate, for example, a bead. In some embodiments, the anti-CD25 antibody, or fragment thereof, is conjugated to a substrate as described herein.

In some embodiments, the T regulatory cells, for example, CD25+ T cells or CD25^{high} T cells, are removed from the population using CD25 depleting reagent from Miltenyi™. In some embodiments, the ratio of cells to CD25 depletion reagent is 1e7 cells to 20 μL, or 1e7 cells to 15 μL, or 1e7 cells to 10 μL, or 1e7 cells to 5 μL, or 1e7 cells to 2.5 μL, or 1e7 cells to 1.25 μL. In some embodiments, for example, for T regulatory cells, greater than 500 million cells/ml is used. In some embodiments, a concentration of cells of 600, 700, 800, or 900 million cells/ml is used.

In some embodiments, the population of immune effector cells to be depleted includes about 6×10⁹ CD25+ T cells. In some embodiments, the population of immune effector cells to be depleted include about 1×10⁹ to 1×10¹⁰ CD25+ T cell, and any integer value in between. In some embodiments, the resulting population T regulatory-depleted cells has 2× 10⁹ T regulatory cells, for example, CD25+ cells or CD25^{high} cells, or less (for example, 1×10⁹, 5×10⁸, 1×10⁸, 5×10⁷, 1×10⁷, or less T regulatory cells).

In some embodiments, the T regulatory cells, for example, CD25+ cells or CD25^{high} cells, are removed from the population using the CliniMAC system with a depletion tubing set, such as, for example, tubing 162-01. In some embodiments, the CliniMAC system is run on a depletion setting such as, for example, DEPLETION2.1.

Without wishing to be bound by a particular theory, decreasing the level of negative regulators of immune cells (for example, decreasing the number of unwanted immune cells, for example, Treg cells), in a subject prior to apheresis or during manufacturing of a CAR-expressing cell product significantly reduces the risk of subject relapse. For example, methods of depleting Treg cells are known in the art. Methods of decreasing Treg cells include, but are not limited to, cyclophosphamide, anti-GITR antibody (an anti-GITR antibody described herein), CD25-depletion, and combinations thereof.

In some embodiments, the manufacturing methods comprise reducing the number of (for example, depleting) Treg cells prior to manufacturing of the CAR-expressing cell. For example, manufacturing methods comprise contacting the sample, for example, the apheresis sample, with an anti-GITR antibody and/or an anti-CD25 antibody (or fragment thereof, or a CD25-binding ligand), for example, to deplete Treg cells prior to manufacturing of the CAR-expressing cell (for example, T cell, NK cell) product.

Without wishing to be bound by a particular theory, decreasing the level of negative regulators of immune cells (for example, decreasing the number of unwanted immune cells, for example, Treg cells), in a subject prior to apheresis or during manufacturing of a CAR-expressing cell product can reduce the risk of a subject's relapse. In some embodiments, a subject is pre-treated with one or more therapies that reduce Treg cells prior to collection of cells for CAR-expressing cell product manufacturing, thereby reducing the risk of subject relapse to CAR-expressing cell treatment. In some embodiments, methods of decreasing Treg cells include, but are not limited to, administration to the subject of one or more of cyclophosphamide, anti-GITR antibody, CD25-depletion, or a combination thereof. In some embodiments, methods of decreasing Treg cells include, but are not limited to, administration to the subject of one or more of cyclophosphamide, anti-GITR antibody, CD25-depletion, or a combination thereof. Administration of one or more of cyclophosphamide, anti-GITR antibody, CD25-depletion, or a combination thereof, can occur before, during or after an infusion of the CAR-expressing cell product. Administration of one or more of cyclophosphamide, anti-GITR antibody, CD25-depletion, or a combination thereof, can occur before, during or after an infusion of the CAR-expressing cell product.

In some embodiments, the manufacturing methods comprise reducing the number of (for example, depleting) Treg cells prior to manufacturing of the CAR-expressing cell. For example, manufacturing methods comprise contacting the sample, for example, the apheresis sample, with an anti-GITR antibody and/or an anti-CD25 antibody (or fragment thereof, or a CD25-binding ligand), for example, to deplete Treg cells prior to manufacturing of the CAR-expressing cell (for example, T cell, NK cell) product.

In some embodiments, a subject is pre-treated with cyclophosphamide prior to collection of cells for CAR-expressing cell product manufacturing, thereby reducing the risk of subject relapse to CAR-expressing cell treatment (for example, CTL019 treatment). In some embodiments, a subject is pre-treated with an anti-GITR antibody prior to collection of cells for CAR-expressing cell (for example, T cell or NK cell) product manufacturing, thereby reducing the risk of subject relapse to CAR-expressing cell treatment.

In some embodiments, the CAR-expressing cell (for example, T cell, NK cell) manufacturing process is modified to deplete Treg cells prior to manufacturing of the CAR-expressing cell (for example, T cell, NK cell) product (for example, a CTL019 product). In some embodiments, CD25-depletion is used to deplete Treg cells prior to manufacturing of the CAR-expressing cell (for example, T cell, NK cell) product (for example, a CTL019 product).

In some embodiments, the population of cells to be removed are neither the regulatory T cells or tumor cells, but cells that otherwise negatively affect the expansion and/or function of CART cells, for example cells expressing CD14, CD11b, CD33, CD15, or other markers expressed by potentially immune suppressive cells. In some embodiments, such cells are envisioned to be removed concurrently with regulatory T cells and/or tumor cells, or following said depletion, or in another order.

The methods described herein can include more than one selection step, for example, more than one depletion step. Enrichment of a T cell population by negative selection can be accomplished, for example, with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, for example, a tumor antigen that does not comprise CD25, for example, CD19, CD30, CD38, CD123, CD20, CD14 or CD11b, to thereby provide a population of T regulatory-depleted, for example, CD25+ depleted or CD25^{high} depleted, and tumor antigen depleted cells that are suitable for expression of a CAR, for example, a CAR described herein. In some embodiments, tumor antigen expressing cells are removed simultaneously with the T regulatory, for example, CD25+ cells or CD25^{high} cells. For example, an anti-CD25 antibody, or fragment thereof, and an anti-tumor antigen antibody, or fragment thereof, can be attached to the same substrate, for example, bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, for example, CD25+ cells or CD25^{high} cells, and the removal of the tumor antigen expressing cells is sequential, and can occur, for example, in either order.

Also provided are methods that include removing cells from the population which express a check point inhibitor, for example, a check point inhibitor described herein, for example, one or more of PD1+ cells, LAG3+ cells, and TIM3+ cells, to thereby provide a population of T regulatory-depleted, for example, CD25+ depleted cells, and check point inhibitor depleted cells, for example, PD1+, LAG3+ and/or TIM3+ depleted cells. Exemplary check point inhibitors include PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (for example, TGF beta), for example, as described herein. In some embodiments, check point inhibitor expressing cells are removed simultaneously with the T regulatory, for example, CD25+ cells or CD25^{high} cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, for example, CD25+ cells or CD25^{high} cells, and the removal of the check point inhibitor expressing cells is sequential, and can occur, for example, in either order.

Methods described herein can include a positive selection step. For example, T cells can isolated by incubation with anti-CD3/anti-CD28 (for example, 3×28)-conjugated beads, such as Dynabeads® M-450 CD3/CD28 T, for a time period sufficient for positive selection of the desired T cells. In some embodiments, the time period is about 30 minutes. In some embodiments, the time period ranges from 30 minutes to 36 hours or longer and all integer values there between. In some embodiments, the time period is at least 1, 2, 3, 4, 5, or 6 hours. In some embodiments, the time period is 10 to 24 hours, for example, 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points.

In some embodiments, a T cell population can be selected that expresses one or more of IFN-γ, TNFα, IL-17A, IL-2, IL-3, IL-4, GM-CSF, IL-10, IL-13, granzyme B, and perforin, or other appropriate molecules, for example, other cytokines. Methods for screening for cell expression can be determined, for example, by the methods described in PCT Publication No.: WO 2013/126712.

For isolation of a desired population of cells by positive or negative selection, the concentration of cells and surface (for example, particles such as beads) can be varied. In some embodiments, it may be desirable to significantly decrease the volume in which beads and cells are mixed together (for example, increase the concentration of cells), to ensure maximum contact of cells and beads. For example, in some embodiments, a concentration of 10 billion cells/ml, 9 billion/ml, 8 billion/ml, 7 billion/ml, 6 billion/ml, or 5 billion/ml is used. In some embodiments, a concentration of 1 billion cells/ml is used. In some embodiments, a concentration of cells from 75, 80, 85, 90, 95, or 100 million cells/ml is used. In some embodiments, concentrations of 125 or 150 million cells/ml can be used.

Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (for example, leukemic blood, tumor tissue, etc.). Such populations of cells may have therapeutic value and would be desirable to obtain. For example, using high concentration of cells allows more efficient selection of CD8+ T cells that normally have weaker CD28 expression.

In some embodiments, it may be desirable to use lower concentrations of cells. By significantly diluting the mixture of T cells and surface (for example, particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In some embodiments, the concentration of cells used is 5×10⁶/ml. In some embodiments, the concentration used can be from about 1×10⁵/ml to 1×10⁶/ml, and any integer value in between.

In some embodiments, the cells may be incubated on a rotator for varying lengths of time at varying speeds at either 2-10° C. or at room temperature.

In some embodiments, a plurality of the immune effector cells of the population do not express diaglycerol kinase (DGK), for example, is DGK-deficient. In some embodiments, a plurality of the immune effector cells of the population do not express Ikaros, for example, is Ikaros-deficient. In some embodiments, a plurality of the immune effector cells of the population do not express DGK and Ikaros, for example, is both DGK and Ikaros-deficient.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some embodiments, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in immune effector cell therapy for any number of diseases or conditions that would benefit from immune effector cell therapy, such as those described herein. In some embodiments a blood sample or an apheresis is taken from a generally healthy subject. In some embodiments, a blood sample or an apheresis is taken from a generally healthy subject who is at risk of developing a disease, but who has not yet developed a disease, and the cells of interest are isolated and frozen for later use. In some embodiments, the T cells may be expanded, frozen, and used at a later time. In some embodiments, samples are collected from a patient shortly after diagnosis of a particular disease as described herein but prior to any treatments. In some embodiments, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In some embodiments of the present invention, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in some embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In some embodiments, the immune effector cells expressing a CAR molecule, for example, a CAR molecule described herein, are obtained from a subject that has received a low, immune enhancing dose of an mTOR inhibitor. In some embodiments, the population of immune effector cells, for example, T cells, to be engineered to express a CAR, are harvested after a sufficient time, or after sufficient dosing of the low, immune enhancing, dose of an mTOR inhibitor, such that the level of PD1 negative immune effector cells, for example, T cells, or the ratio of PD1 negative immune effector cells, for example, T cells/PD1 positive immune effector cells, for example, T cells, in the subject or harvested from the subject has been, at least transiently, increased.

In other embodiments, population of immune effector cells, for example, T cells, which have, or will be engineered to express a CAR, can be treated ex vivo by contact with an amount of an mTOR inhibitor that increases the number of PD1 negative immune effector cells, for example, T cells or increases the ratio of PD1 negative immune effector cells, for example, T cells/PD1 positive immune effector cells, for example, T cells.

It is recognized that the methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS™ Immune Cell Serum Replacement” Clinical & Translational Immunology (2015) 4, e31; doi: 10.1038/cti.2014.31.

In some embodiments, the methods of the application can utilize media conditions comprising at least about 0.1%, 0.5%, 1.0%, 1.5%, 2%, 2.5%, 3%, 3.5%, 4%, 4.5%, 5%, 6%, 7%, 8%, 9% or 10% serum. In some embodiments, the media comprises about 0.5%-5%, about 0.5%-4.5%, about 0.5%-4%, about 0.5%-3.5%, about 0.5%-3%, about 0.5%-2.5%, about 0.5%-2%, about 0.5%-1.5%, about 0.5%-1.0%, about 1.0%-5%, about 1.5%-5%, about 2%-5%, about 2.5%-5%, about 3%-5%, about 3.5%-5%, about 4%-5%, or about 4.5%-5% serum. In some embodiments, the media comprises about 0.5% serum. In some embodiments, the media comprises about 0.5% serum. In some embodiments, the media comprises about 1% serum. In some embodiments, the media comprises about 1.5% serum. In some embodiments, the media comprises about 2% serum. In some embodiments, the media comprises about 2.5% serum. In some embodiments, the media comprises about 3% serum. In some embodiments, the media comprises about 3.5% serum. In some embodiments, the media comprises about 4% serum. In some embodiments, the media comprises about 4.5% serum. In some embodiments, the media comprises about 5% serum. In some embodiments, the serum comprises human serum, e.g., human AB serum. In some embodiments, the serum is human serum that has been allowed to naturally coagulate after collection, e.g., off-the-clot (OTC) serum. In some embodiments, the serum is plasma-derived serum human serum. Plasma-derived serum can be produced by defibrinating pooled human plasma collected in the presence of an anticoagulant, e.g., sodium citrate.

In some embodiments, the methods of the application can utilize culture media conditions comprising serum-free medium. In some embodiments, the serum free medium is OpTmizer™ CTS™ (LifeTech), Immunocult™ XF (Stemcell technologies), CellGro™ (CellGenix), TexMacs™ (Miltenyi), Stemline™ (Sigma), Xvivo15™ (Lonza), PrimeXV® (Irvine Scientific), or StemXVivo® (RandD systems). The serum-free medium can be supplemented with a serum substitute such as ICSR (immune cell serum replacement) from LifeTech. The level of serum substitute (for example, ICSR) can be, for example, up to 5%, for example, about 1%, 2%, 3%, 4%, or 5%. In some embodiments, the serum-free medium can be supplemented with serum, e.g., human serum, e.g., human AB serum. In some embodiments, the serum is human serum that has been allowed to naturally coagulate after collection, e.g., off-the-clot (OTC) serum. In some embodiments, the serum is plasma-derived human serum. Plasma-derived serum can be produced by defibrinating pooled human plasma collected in the presence of an anticoagulant, e.g., sodium citrate.

In some embodiments, a T cell population is diaglycerol kinase (DGK)-deficient. DGK-deficient cells include cells that do not express DGK RNA or protein, or have reduced or inhibited DGK activity. DGK-deficient cells can be generated by genetic approaches, for example, administering RNA-interfering agents, for example, siRNA, shRNA, miRNA, to reduce or prevent DGK expression. Alternatively, DGK-deficient cells can be generated by treatment with DGK inhibitors described herein.

In some embodiments, a T cell population is Ikaros-deficient. Ikaros-deficient cells include cells that do not express Ikaros RNA or protein, or have reduced or inhibited Ikaros activity, Ikaros-deficient cells can be generated by genetic approaches, for example, administering RNA-interfering agents, for example, siRNA, shRNA, miRNA, to reduce or prevent Ikaros expression. Alternatively, Ikaros-deficient cells can be generated by treatment with Ikaros inhibitors, for example, lenalidomide.

In embodiments, a T cell population is DGK-deficient and Ikaros-deficient, for example, does not express DGK and Ikaros, or has reduced or inhibited DGK and Ikaros activity. Such DGK and Ikaros-deficient cells can be generated by any of the methods described herein.

In some embodiments, the NK cells are obtained from the subject. In some embodiments, the NK cells are an NK cell line, for example, NK-92 cell line (Conkwest).

### Allogeneic CAR-Expressing Cells

In embodiments described herein, the immune effector cell can be an allogeneic immune effector cell, for example, T cell or NK cell. For example, the cell can be an allogeneic T cell, for example, an allogeneic T cell lacking expression of a functional T cell receptor (TCR) and/or human leukocyte antigen (HLA), for example, HLA class I and/or HLA class II.

A T cell lacking a functional TCR can be, for example, engineered such that it does not express any functional TCR on its surface, engineered such that it does not express one or more subunits that comprise a functional TCR (for example, engineered such that it does not express (or exhibits reduced expression) of TCR alpha, TCR beta, TCR gamma, TCR delta, TCR epsilon, and/or TCR zeta) or engineered such that it produces very little functional TCR on its surface. Alternatively, the T cell can express a substantially impaired TCR, for example, by expression of mutated or truncated forms of one or more of the subunits of the TCR. The term “substantially impaired TCR” means that this TCR will not elicit an adverse immune reaction in a host.

A T cell described herein can be, for example, engineered such that it does not express a functional HLA on its surface. For example, a T cell described herein, can be engineered such that cell surface expression HLA, for example, HLA class 1 and/or HLA class II, is downregulated. In some embodiments, downregulation of HLA may be accomplished by reducing or eliminating expression of beta-2 microglobulin (B2M).

In some embodiments, the T cell can lack a functional TCR and a functional HLA, for example, HLA class I and/or HLA class II.

Modified T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

In some embodiments, the allogeneic cell can be a cell which does not express or expresses at low levels an inhibitory molecule, for example by any method described herein. For example, the cell can be a cell that does not express or expresses at low levels an inhibitory molecule, for example, that can decrease the ability of a CAR-expressing cell to mount an immune effector response. Examples of inhibitory molecules include PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (for example, TGF beta). Inhibition of an inhibitory molecule, for example, by inhibition at the DNA, RNA or protein level, can optimize a CAR-expressing cell performance. In embodiments, an inhibitory nucleic acid, for example, an inhibitory nucleic acid, for example, a dsRNA, for example, an siRNA or shRNA, a clustered regularly interspaced short palindromic repeats (CRISPR), a transcription-activator like effector nuclease (TALEN), or a zinc finger endonuclease (ZFN), for example, as described herein, can be used.

siRNA and shRNA to Inhibit TCR or HLA

In some embodiments, TCR expression and/or HLA expression can be inhibited using siRNA or shRNA that targets a nucleic acid encoding a TCR and/or HLA, and/or an inhibitory molecule described herein (for example, PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, for example, T cell.

Expression systems for siRNA and shRNAs, and exemplary shRNAs, are described, for example, in paragraphs 649 and 650 of International Application WO2015/142675, filed Mar. 13, 2015, which is incorporated by reference in its entirety.

### CRISPR to Inhibit TCR or HLA

“CRISPR” or “CRISPR to TCR and/or HLA” or “CRISPR to inhibit TCR and/or HLA” as used herein refers to a set of clustered regularly interspaced short palindromic repeats, or a system comprising such a set of repeats. “Cas”, as used herein, refers to a CRISPR-associated protein. A “CRISPR/Cas” system refers to a system derived from CRISPR and Cas which can be used to silence or mutate a TCR and/or HLA gene, and/or an inhibitory molecule described herein (for example, PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, for example, T cell.

The CRISPR/Cas system, and uses thereof, are described, for example, in paragraphs 651-658 of International Application WO2015/142675, filed Mar. 13, 2015, which is incorporated by reference in its entirety.

TALEN to Inhibit TCR and/or HLA

“TALEN” or “TALEN to HLA and/or TCR” or “TALEN to inhibit HLA and/or TCR” refers to a transcription activator-like effector nuclease, an artificial nuclease which can be used to edit the HLA and/or TCR gene, and/or an inhibitory molecule described herein (for example, PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, for example, T cell.

TALENs, and uses thereof, are described, for example, in paragraphs 659-665 of International Application WO2015/142675, filed Mar. 13, 2015, which is incorporated by reference in its entirety.

Zinc Finger Nuclease to Inhibit HLA and/or TCR

“ZFN” or “Zinc Finger Nuclease” or “ZFN to HLA and/or TCR” or “ZFN to inhibit HLA and/or TCR” refer to a zinc finger nuclease, an artificial nuclease which can be used to edit the HLA and/or TCR gene, and/or an inhibitory molecule described herein (for example, PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, for example, T cell.

ZFNs, and uses thereof, are described, for example, in paragraphs 666-671 of International Application WO2015/142675, filed Mar. 13, 2015, which is incorporated by reference in its entirety.

### Telomerase Expression

Telomeres play a crucial role in somatic cell persistence, and their length is maintained by telomerase (TERT). Telomere length in CLL cells may be very short (Roth et al., “Significantly shorter telomeres in T-cells of patients with ZAP-70+/CD38 chronic lymphocytic leukaemia” British Journal of Haematology, 143, 383-386, Aug. 28, 2008), and may be even shorter in manufactured CAR-expressing cells, for example, CART19 cells, limiting their potential to expand after adoptive transfer to a patient. Telomerase expression can rescue CAR-expressing cells from replicative exhaustion.

While not wishing to be bound by any particular theory, in some embodiments, a therapeutic T cell has short term persistence in a patient, due to shortened telomeres in the T cell; accordingly, transfection with a telomerase gene can lengthen the telomeres of the T cell and improve persistence of the T cell in the patient. See Carl June, “Adoptive T cell therapy for cancer in the clinic”, Journal of Clinical Investigation, 117:1466-1476 (2007). Thus, in some embodiments, an immune effector cell, for example, a T cell, ectopically expresses a telomerase subunit, for example, the catalytic subunit of telomerase, for example, TERT, for example, hTERT. In some embodiments, this disclosure provides a method of producing a CAR-expressing cell, comprising contacting a cell with a nucleic acid encoding a telomerase subunit, for example, the catalytic subunit of telomerase, for example, TERT, for example, hTERT. The cell may be contacted with the nucleic acid before, simultaneous with, or after being contacted with a construct encoding a CAR.

Telomerase expression may be stable (for example, the nucleic acid may integrate into the cell's genome) or transient (for example, the nucleic acid does not integrate, and expression declines after a period of time, for example, several days). Stable expression may be accomplished by transfecting or transducing the cell with DNA encoding the telomerase subunit and a selectable marker, and selecting for stable integrants. Alternatively or in combination, stable expression may be accomplished by site-specific recombination, for example, using the Cre/Lox or FLP/FRT system.

Transient expression may involve transfection or transduction with a nucleic acid, for example, DNA or RNA such as mRNA. In some embodiments, transient mRNA transfection avoids the genetic instability sometimes associated with stable transfection with TERT. Transient expression of exogenous telomerase activity is described, for example, in International Application WO2014/130909, which is incorporated by reference herein in its entirety. In embodiments, mRNA-based transfection of a telomerase subunit is performed according to the messenger RNA Therapeutics™ platform commercialized by Moderna Therapeutics. For instance, the method may be a method described in U.S. Pat. Nos. 8,710,200, 8,822,663, 8,680,069, 8,754,062, 8,664,194, or 8,680,069.

In some embodiments, hTERT has the amino acid sequence of GenBank Protein ID AAC51724.1 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795):

(SEQ ID NO: 284)
MPRAPRCRAVRSLLRSHYREVLPLATFVRRLGPQGWRLVQRGDPAAFRA
LVAQCLVCVPWDARPPPAAPSFRQVSCLKELVARVLQRLCERGAKNVLA
FGFALLDGARGGPPEAFTTSVRSYLPNTVTDALRGSGAWGLLLRRVGDD
VLVHLLARCALFVLVAPSCAYQVCGPPLYQLGAATQARPPPHASGPRRR
LGCERAWNHSVREAGVPLGLPAPGARRRGGSASRSLPLPKRPRRGAAPE
PERTPVGQGSWAHPGRTRGPSDRGFCVVSPARPAEEATSLEGALSGTRH
SHPSVGRQHHAGPPSTSRPPRPWDTPCPPVYAETKHFLYSSGDKEQLRP
SFLLSSLRPSLTGARRLVETIFLGSRPWMPGTPRRLPRLPQRYWQMRPL
FLELLGNHAQCPYGVLLKTHCPLRAAVTPAAGVCAREKPQGSVAAPEEE
DTDPRRLVQLLRQHSSPWQVYGFVRACLRRLVPPGLWGSRHNERRFLRN
TKKFISLGKHAKLSLQELTWKMSVRGCAWLRRSPGVGCVPAAEHRLREE
ILAKFLHWLMSVYVVELLRSFFYVTETTFQKNRLFFYRKSVWSKLQSIG
IRQHLKRVQLRELSEAEVRQHREARPALLTSRLRFIPKPDGLRPIVNMD
YVVGARTFRREKRAERLTSRVKALFSVLNYERARRPGLLGASVLGLDDI
HRAWRTFVLRVRAQDPPPELYFVKVDVTGAYDTIPQDRLTEVIASIIKP
QNTYCVRRYAVVQKAAHGHVRKAFKSHVSTLTDLQPYMRQFVAHLQETS
PLRDAVVIEQSSSLNEASSGLFDVFLRFMCHHAVRIRGKSYVQCQGIPQ
GSILSTLLCSLCYGDMENKLFAGIRRDGLLLRLVDDFLLVTPHLTHAKT
FLRTLVRGVPEYGCVVNLRKTVVNFPVEDEALGGTAFVQMPAHGLFPWC
GLLLDTRTLEVQSDYSSYARTSIRASLTFNRGFKAGRNMRRKLFGVLRL
KCHSLFLDLQVNSLQTVCTNIYKILLLQAYRFHACVLQLPFHQQVWKNP
TFFLRVISDTASLCYSILKAKNAGMSLGAKGAAGPLPSEAVQWLCHQAF
LLKLTRHRVTYVPLLGSLRTAQTQLSRKLPGTTLTALEAAANPALPSDF
KTILD

In some embodiments, the hTERT has a sequence at least 80%, 85%, 90%, 95%, 96%, 97%, 98%, or 99% identical to the sequence of SEQ ID NO: 284. In some embodiments, the hTERT has a sequence of SEQ ID NO: 284. In some embodiments, the hTERT comprises a deletion (for example, of no more than 5, 10, 15, 20, or 30 amino acids) at the N-terminus, the C-terminus, or both. In some embodiments, the hTERT comprises a transgenic amino acid sequence (for example, of no more than 5, 10, 15, 20, or 30 amino acids) at the N-terminus, the C-terminus, or both.

In some embodiments, the hTERT is encoded by the nucleic acid sequence of GenBank Accession No. AF018167 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795).

### Activation and Expansion of Immune Effector Cells (for Example, T Cells)

Immune effector cells such as T cells generated or enriched by the methods described herein may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, a population of immune effector cells may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (for example, bryostatin) in conjunction with a calcium ionophore. For costimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besanç on, France) can be used as can other methods commonly known in the art (Berg et al., Transplant Proc. 30 (8): 3975-3977, 1998; Haanen et al., J. Exp. Med. 190 (9): 13191328, 1999; Garland et al., J. Immunol Meth. 227 (1-2): 53-63, 1999).

In some embodiments, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In some embodiments, the agent providing the costimulatory signal is bound to a cell surface and the agent providing the primary activation signal is in solution or coupled to a surface. In some embodiments, both agents can be in solution. In some embodiments, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In some embodiments, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In some embodiments, a 1:1 ratio of each antibody bound to the beads for CD4+ T cell expansion and T cell growth is used. In some embodiments of the present invention, a ratio of anti CD3:CD28 antibodies bound to the beads is used such that an increase in T cell expansion is observed as compared to the expansion observed using a ratio of 1:1. In some embodiments an increase of from about 1 to about 3 fold is observed as compared to the expansion observed using a ratio of 1:1. In some embodiments, the ratio of CD3:CD28 antibody bound to the beads ranges from 100:1 to 1:100 and all integer values there between. In some embodiments, more anti-CD28 antibody is bound to the particles than anti-CD3 antibody, i.e., the ratio of CD3:CD28 is less than one. In some embodiments, the ratio of anti CD28 antibody to anti CD3 antibody bound to the beads is greater than 2:1. In some embodiments, a 1:100 CD3:CD28 ratio of antibody bound to beads is used. In some embodiments, a 1:75 CD3:CD28 ratio of antibody bound to beads is used. In some embodiments, a 1:50 CD3:CD28 ratio of antibody bound to beads is used. In some embodiments, a 1:30 CD3:CD28 ratio of antibody bound to beads is used. In some embodiments, a 1:10 CD3:CD28 ratio of antibody bound to beads is used. In some embodiments, a 1:3 CD3:CD28 ratio of antibody bound to the beads is used. In some embodiments, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In some embodiments the ratio of cells to particles ranges from 1:100 to 100:1 and any integer values in-between and in some embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain suitable values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one suitable ratio being at least 1:1 particles per T cell. In some embodiments, a ratio of particles to cells of 1:1 or less is used. In some embodiments, a suitable particle: cell ratio is 1:5. In some embodiments, the ratio of particles to cells can be varied depending on the day of stimulation. For example, in some embodiments, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In some embodiments, the ratio of particles to cells is 1:1 on the first day of stimulation and adjusted to 1:5 on the third and fifth days of stimulation. In some embodiments, particles are added on a daily or every other day basis to a final ratio of 1:1 on the first day, and 1:5 on the third and fifth days of stimulation. In some embodiments, the ratio of particles to cells is 2:1 on the first day of stimulation and adjusted to 1:10 on the third and fifth days of stimulation. In some embodiments, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type. In some embodiments, the most typical ratios for use are in the neighborhood of 1:1, 2:1 and 3:1 on the first day.

In some embodiments, the cells, such as T cells, are combined with agent-coated beads, the beads and the cells are subsequently separated, and then the cells are cultured. In some embodiments, prior to culture, the agent-coated beads and cells are not separated but are cultured together. In some embodiments, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In some embodiments the cells (for example, 10⁴ to 10⁹ T cells) and beads (for example, Dynabeads® M-450 CD3/CD28 T paramagnetic beads at a ratio of 1:1) are combined in a buffer, for example PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In some embodiments, it may be desirable to significantly decrease the volume in which particles and cells are mixed together (i.e., increase the concentration of cells), to ensure maximum contact of cells and particles. For example, in some embodiments, a concentration of about 10 billion cells/ml, 9 billion/ml, 8 billion/ml, 7 billion/ml, 6 billion/ml, 5 billion/ml, or 2 billion cells/ml is used. In some embodiments, greater than 100 million cells/ml is used. In some embodiments, a concentration of cells of 10, 15, 20, 25, 30, 35, 40, 45, or 50 million cells/ml is used. In some embodiments, a concentration of cells from 75, 80, 85, 90, 95, or 100 million cells/ml is used. In some embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in some embodiments. For example, using high concentration of cells allows more efficient selection of CD8+ T cells that normally have weaker CD28 expression.

In some embodiments, cells transduced with a nucleic acid encoding a CAR, for example, a CAR described herein, for example, a CD19 CAR described herein, are expanded, for example, by a method described herein. In some embodiments, the cells are expanded in culture for a period of several hours (for example, about 2, 3, 4, 5, 6, 7, 8, 9, 10, 15, 18, 21 hours) to about 14 days (for example, 1, 2, 3, 4, 5, 6, 7, 8, 9, 10, 11, 12, 13 or 14 days). In some embodiments, the cells are expanded for a period of 4 to 9 days. In some embodiments, the cells are expanded for a period of 8 days or less, for example, 7, 6 or 5 days. In some embodiments, the cells are expanded in culture for 5 days, and the resulting cells are more potent than the same cells expanded in culture for 9 days under the same culture conditions. Potency can be defined, for example, by various T cell functions, for example proliferation, target cell killing, cytokine production, activation, migration, surface CAR expression, CAR quantitative PCR, or combinations thereof. In some embodiments, the cells, for example, a CD19 CAR cell described herein, expanded for 5 days show at least a one, two, three or four-fold increase in cells doublings upon antigen stimulation as compared to the same cells expanded in culture for 9 days under the same culture conditions. In some embodiments, the cells, for example, the cells expressing a CD19 CAR described herein, are expanded in culture for 5 days, and the resulting cells exhibit higher proinflammatory cytokine production, for example, IFN-γ and/or GM-CSF levels, as compared to the same cells expanded in culture for 9 days under the same culture conditions. In some embodiments, the cells, for example, a CD19 CAR cell described herein, expanded for 5 days show at least a one, two, three, four, five, ten-fold or more increase in pg/ml of proinflammatory cytokine production, for example, IFN-γ and/or GM-CSF levels, as compared to the same cells expanded in culture for 9 days under the same culture conditions.

Several cycles of stimulation may also be desired such that culture time of T cells can be 60 days or more. Conditions appropriate for T cell culture include an appropriate media (for example, Minimal Essential Media, α-MEM, RPMI Media 1640, AIM-V, DMEM, F-12, or X-vivo 15 (Lonza), X-Vivo 20, OpTmizer, and IMDM) that may contain factors necessary for proliferation and viability, including serum (for example, fetal bovine or human serum), interleukin-2 (IL-2), insulin, IFNγ, IL-4, IL-7, GM-CSF, IL-10, IL-12, IL-15, TGFβ, and TNFα or any other additives for the growth of cells known to the skilled artisan. Other additives for the growth of cells include, but are not limited to, surfactant, plasmanate, and reducing agents such as N-acetyl-cysteine and 2-mercaptoethanol. Media can include, but is not limited to RPMI 1640, AIM-V, DMEM, MEM, α-MEM, F-12, X-Vivo 15, X-Vivo 20, OpTmizer, and IMDM with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, for example, penicillin and streptomycin, are included only in experimental cultures, not in cultures of cells that are to be infused into a subject. The target cells are maintained under conditions necessary to support growth, for example, an appropriate temperature (for example, 37° C.) and atmosphere (for example, air plus 5% CO₂).

In some embodiments, the cells are expanded in an appropriate media (for example, media described herein) that includes one or more interleukin that result in at least a 200-fold (for example, 200-fold, 250-fold, 300-fold, 350-fold) increase in cells over a 14-day expansion period, for example, as measured by a method described herein such as flow cytometry. In some embodiments, the cells are expanded in the presence IL-15 and/or IL-7 (for example, IL-15 and IL-7).

In embodiments, methods described herein, for example, CAR-expressing cell manufacturing methods, comprise removing T regulatory cells, for example, CD25+ T cells or CD25^{high} T cells, from a cell population, for example, using an anti-CD25 antibody, or fragment thereof, or a CD25-binding ligand, IL-2. Methods of removing T regulatory cells, for example, CD25+ T cells or CD25^{high} T cells, from a cell population are described herein. In embodiments, the methods, for example, manufacturing methods, further comprise contacting a cell population (for example, a cell population in which T regulatory cells, such as CD25+ T cells or CD25^{high} T cells, have been depleted; or a cell population that has previously contacted an anti-CD25 antibody, fragment thereof, or CD25-binding ligand) with IL-15 and/or IL-7. For example, the cell population (for example, that has previously contacted an anti-CD25 antibody, fragment thereof, or CD25-binding ligand) is expanded in the presence of IL-15 and/or IL-7.

In some embodiments a CAR-expressing cell described herein is contacted with a composition comprising a interleukin-15 (IL-15) polypeptide, a interleukin-15 receptor alpha (IL-15Ra) polypeptide, or a combination of both a IL-15 polypeptide and a IL-15Ra polypeptide for example, hetIL-15, during the manufacturing of the CAR-expressing cell, for example, ex vivo. In embodiments, a CAR-expressing cell described herein is contacted with a composition comprising a IL-15 polypeptide during the manufacturing of the CAR-expressing cell, for example, ex vivo. In embodiments, a CAR-expressing cell described herein is contacted with a composition comprising a combination of both a IL-15 polypeptide and a IL-15 Ra polypeptide during the manufacturing of the CAR-expressing cell, for example, ex vivo. In embodiments, a CAR-expressing cell described herein is contacted with a composition comprising hetIL-15 during the manufacturing of the CAR-expressing cell, for example, ex vivo.

In some embodiments the CAR-expressing cell described herein is contacted with a composition comprising hetIL-15 during ex vivo expansion. In some embodiments, the CAR-expressing cell described herein is contacted with a composition comprising an IL-15 polypeptide during ex vivo expansion. In some embodiments, the CAR-expressing cell described herein is contacted with a composition comprising both an IL-15 polypeptide and an IL-15Ra polypeptide during ex vivo expansion. In some embodiments the contacting results in the survival and proliferation of a lymphocyte subpopulation, for example, CD8+ T cells.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once a CAR described herein is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of a CAR of the present invention are described in further detail below

Western blot analysis of CAR expression in primary T cells can be used to detect the presence of monomers and dimers, for example, as described in paragraph 695 of International Application WO2015/142675, filed Mar. 13, 2015, which is herein incorporated by reference in its entirety.

In vitro expansion of CAR+ T cells following antigen stimulation can be measured by flow cytometry. For example, a mixture of CD4⁺ and CD8⁺ T cells are stimulated with αCD3/αCD28 aAPCs followed by transduction with lentiviral vectors expressing GFP under the control of the promoters to be analyzed. Exemplary promoters include the CMV IE gene, EF-1a, ubiquitin C, or phosphoglycerokinase (PGK) promoters. GFP fluorescence is evaluated on day 6 of culture in the CD4⁺ and/or CD8⁺ T cell subsets by flow cytometry. See, for example, Milone et al., Molecular Therapy 17 (8): 1453-1464 (2009). Alternatively, a mixture of CD4⁺ and CD8⁺ T cells are stimulated with αCD3/αCD28 coated magnetic beads on day 0, and transduced with CAR on day 1 using a bicistronic lentiviral vector expressing CAR along with eGFP using a 2A ribosomal skipping sequence. Cultures are re-stimulated with either a cancer associated antigen as described herein⁺ K562 cells (K562-expressing a cancer associated antigen as described herein), wild-type K562 cells (K562 wild type) or K562 cells expressing hCD32 and 4-1BBL in the presence of antiCD3 and anti-CD28 antibody (K562-BBL-3/28). Exogenous IL-2 is added to the cultures every other day at 100 IU/ml. GFP⁺ T cells are enumerated by flow cytometry using bead-based counting. See, for example, Milone et al., Molecular Therapy 17 (8): 1453-1464 (2009).

Sustained CAR⁺ T cell expansion in the absence of re-stimulation can also be measured. See, for example, Milone et al., Molecular Therapy 17 (8): 1453-1464 (2009). Briefly, mean T cell volume (fl) is measured on day 8 of culture using a Coulter Multisizer III particle counter or a higher version, a Nexcelom Cellometer Vision, Millipore Scepter or other cell counters, following stimulation with αCD3/αCD28 coated magnetic beads on day 0, and transduction with the indicated CAR on day 1.

Animal models can also be used to measure a CAR-expressing cell activity, for example, as described in paragraph 698 of International Application WO2015/142675, filed Mar. 13, 2015, which is herein incorporated by reference in its entirety.

Dose dependent CAR treatment response can be evaluated, for example, as described in paragraph 699 of International Application WO2015/142675, filed Mar. 13, 2015, which is herein incorporated by reference in its entirety.

Assessment of cell proliferation and cytokine production has been previously described, as described in paragraph 700 of International Application WO2015/142675, filed Mar. 13, 2015, which is herein incorporated by reference in its entirety.

Cytotoxicity can be assessed by a standard 51Cr-release assay, for example, as described in paragraph 701 of International Application WO2015/142675, filed Mar. 13, 2015, which is herein incorporated by reference in its entirety. Alternative non-radioactive methods can be utilized as well.

Cytotoxicity can also be assessed by measuring changes in adherent cell's electrical impedance, for example, using an xCELLigence real time cell analyzer (RTCA). In some embodiments, cytotoxicity is measured at multiple time points.

Imaging technologies can be used to evaluate specific trafficking and proliferation of CARs in tumor-bearing animal models, for example, as described in paragraph 702 of International Application WO2015/142675, filed Mar. 13, 2015, which is herein incorporated by reference in its entirety.

Other assays, including those described in the Example section herein as well as those that are known in the art can also be used to evaluate the CARs described herein.

Alternatively, or in combination to the methods disclosed herein, methods and compositions for one or more of: detection and/or quantification of CAR-expressing cells (for example, in vitro or in vivo (for example, clinical monitoring)); immune cell expansion and/or activation; and/or CAR-specific selection, that involve the use of a CAR ligand, are disclosed. In some embodiments, the CAR ligand is an antibody that binds to the CAR molecule, for example, binds to the extracellular antigen binding domain of CAR (for example, an antibody that binds to the antigen binding domain, for example, an anti-idiotypic antibody; or an antibody that binds to a constant region of the extracellular binding domain). In other embodiments, the CAR ligand is a CAR antigen molecule (for example, a CAR antigen molecule as described herein).

In some embodiments, a method for detecting and/or quantifying CAR-expressing cells is disclosed. For example, the CAR ligand can be used to detect and/or quantify CAR-expressing cells in vitro or in vivo (for example, clinical monitoring of CAR-expressing cells in a patient, or dosing a patient). The method includes:
providing the CAR ligand (optionally, a labelled CAR ligand, for example, a CAR ligand that includes a tag, a bead, a radioactive or fluorescent label);
acquiring the CAR-expressing cell (for example, acquiring a sample containing CAR-expressing cells, such as a manufacturing sample or a clinical sample);
contacting the CAR-expressing cell with the CAR ligand under conditions where binding occurs, thereby detecting the level (for example, amount) of the CAR-expressing cells present. Binding of the CAR-expressing cell with the CAR ligand can be detected using standard techniques such as FACS, ELISA and the like.

In some embodiments, a method of expanding and/or activating cells (for example, immune effector cells) is disclosed. The method includes:
providing a CAR-expressing cell (for example, a first CAR-expressing cell or a transiently expressing CAR cell);
contacting said CAR-expressing cell with a CAR ligand, for example, a CAR ligand as described herein), under conditions where immune cell expansion and/or proliferation occurs, thereby producing the activated and/or expanded cell population.

In certain embodiments, the CAR ligand is present on a substrate (for example, is immobilized or attached to a substrate, for example, a non-naturally occurring substrate). In some embodiments, the substrate is a non-cellular substrate. The non-cellular substrate can be a solid support chosen from, for example, a plate (for example, a microtiter plate), a membrane (for example, a nitrocellulose membrane), a matrix, a chip or a bead. In embodiments, the CAR ligand is present in the substrate (for example, on the substrate surface). The CAR ligand can be immobilized, attached, or associated covalently or non-covalently (for example, cross-linked) to the substrate. In some embodiments, the CAR ligand is attached (for example, covalently attached) to a bead. In the aforesaid embodiments, the immune cell population can be expanded in vitro or ex vivo. The method can further include culturing the population of immune cells in the presence of the ligand of the CAR molecule, for example, using any of the methods described herein.

In other embodiments, the method of expanding and/or activating the cells further comprises addition of a second stimulatory molecule, for example, CD28. For example, the CAR ligand and the second stimulatory molecule can be immobilized to a substrate, for example, one or more beads, thereby providing increased cell expansion and/or activation.

In some embodiments, a method for selecting or enriching for a CAR expressing cell is provided. The method includes contacting the CAR expressing cell with a CAR ligand as described herein; and selecting the cell on the basis of binding of the CAR ligand.

In yet other embodiments, a method for depleting, reducing and/or killing a CAR expressing cell is provided. The method includes contacting the CAR expressing cell with a CAR ligand as described herein; and targeting the cell on the basis of binding of the CAR ligand, thereby reducing the number, and/or killing, the CAR-expressing cell. In some embodiments, the CAR ligand is coupled to a toxic agent (for example, a toxin or a cell ablative drug). In some embodiments, the anti-idiotypic antibody can cause effector cell activity, for example, ADCC or ADC activities.

Exemplary anti-CAR antibodies that can be used in the methods disclosed herein are described, for example, in WO 2014/190273 and by Jena et al., “Chimeric Antigen Receptor (CAR)-Specific Monoclonal Antibody to Detect CD19-Specific T cells in Clinical Trials”, PLOS March 2013 8:3 e57838, the contents of which are incorporated by reference.

In some embodiments, the compositions and methods herein are optimized for a specific subset of T cells, for example, as described in US Ser. No. PCT/US2015/043219 filed Jul. 31, 2015, the contents of which are incorporated herein by reference in their entirety. In some embodiments, the optimized subsets of T cells display an enhanced persistence compared to a control T cell, for example, a T cell of a different type (for example, CD8+ or CD4+) expressing the same construct.

In some embodiments, a CD4+ T cell comprises a CAR described herein, which CAR comprises an intracellular signaling domain suitable for (for example, optimized for, for example, leading to enhanced persistence in) a CD4+ T cell, for example, an ICOS domain. In some embodiments, a CD8+ T cell comprises a CAR described herein, which CAR comprises an intracellular signaling domain suitable for (for example, optimized for, for example, leading to enhanced persistence of) a CD8+ T cell, for example, a 4-1BB domain, a CD28 domain, or another costimulatory domain other than an ICOS domain. In some embodiments, the CAR described herein comprises an antigen binding domain described herein, for example, a CAR comprising an antigen binding domain.

In some embodiments, described herein is a method of treating a subject, for example, a subject having cancer. The method includes administering to said subject, an effective amount of:
1) a CD4+ T cell comprising a CAR (the CARCD4+) comprising:
an antigen binding domain, for example, an antigen binding domain described herein;
a transmembrane domain; and
an intracellular signaling domain, for example, a first costimulatory domain, for example, an ICOS domain; and
2) a CD8+ T cell comprising a CAR (the CARCD8+) comprising:
an antigen binding domain, for example, an antigen binding domain described herein;
a transmembrane domain; and
an intracellular signaling domain, for example, a second costimulatory domain, for example, a 4-1BB domain, a CD28 domain, or another costimulatory domain other than an ICOS domain;
wherein the CARCD4+ and the CARCD8+ differ from one another.
   Optionally, the method further includes administering:
3) a second CD8+ T cell comprising a CAR (the second CARCD8+) comprising:
an antigen binding domain, for example, an antigen binding domain described herein;
a transmembrane domain; and
an intracellular signaling domain, wherein the second CARCD8+ comprises an intracellular signaling domain, for example, a costimulatory signaling domain, not present on the CARCD8+, and, optionally, does not comprise an ICOS signaling domain.

### Biopolymer Delivery Methods

In some embodiments, one or more CAR-expressing cells as disclosed herein can be administered or delivered to the subject via a biopolymer scaffold, for example, a biopolymer implant. Biopolymer scaffolds can support or enhance the delivery, expansion, and/or dispersion of the CAR-expressing cells described herein. A biopolymer scaffold comprises a biocompatible (for example, does not substantially induce an inflammatory or immune response) and/or a biodegradable polymer that can be naturally occurring or synthetic. Exemplary biopolymers are described, for example, in paragraphs 1004-1006 of International Application WO2015/142675, filed Mar. 13, 2015, which is herein incorporated by reference in its entirety.

### Pharmaceutical Compositions and Treatments

In some embodiments, the disclosure provides a method of treating a patient, comprising administering CAR-expressing cells produced as described herein, optionally in combination with one or more other therapies. In some embodiments, the disclosure provides a method of treating a patient, comprising administering a reaction mixture comprising CAR-expressing cells as described herein, optionally in combination with one or more other therapies. In some embodiments, the disclosure provides a method of shipping or receiving a reaction mixture comprising CAR-expressing cells as described herein. In some embodiments, the disclosure provides a method of treating a patient, comprising receiving a CAR-expressing cell that was produced as described herein, and further comprising administering the CAR-expressing cell to the patient, optionally in combination with one or more other therapies. In some embodiments, the disclosure provides a method of treating a patient, comprising producing a CAR-expressing cell as described herein, and further comprising administering the CAR-expressing cell to the patient, optionally in combination with one or more other therapies. The other therapy may be, for example, a cancer therapy such as chemotherapy.

In some embodiments, cells expressing a CAR described herein are administered to a subject in combination with a molecule that decreases the Treg cell population. Methods that decrease the number of (for example, deplete) Treg cells are known in the art and include, for example, CD25 depletion, cyclophosphamide administration, modulating GITR function. Without wishing to be bound by theory, it is believed that reducing the number of Treg cells in a subject prior to apheresis or prior to administration of a CAR-expressing cell described herein reduces the number of unwanted immune cells (for example, Tregs) in the tumor microenvironment and reduces the subject's risk of relapse.

In some embodiments, a therapy described herein, for example, a CAR-expressing cell, is administered to a subject in combination with a molecule targeting GITR and/or modulating GITR functions, such as a GITR agonist and/or a GITR antibody that depletes regulatory T cells (Tregs). In embodiments, cells expressing a CAR described herein are administered to a subject in combination with cyclophosphamide. In some embodiments, the GITR binding molecules and/or molecules modulating GITR functions (for example, GITR agonist and/or Treg depleting GITR antibodies) are administered prior to the CAR-expressing cell. For example, in some embodiments, a GITR agonist can be administered prior to apheresis of the cells. In embodiments, cyclophosphamide is administered to the subject prior to administration (for example, infusion or re-infusion) of the CAR-expressing cell or prior to apheresis of the cells. In embodiments, cyclophosphamide and an anti-GITR antibody are administered to the subject prior to administration (for example, infusion or re-infusion) of the CAR-expressing cell or prior to apheresis of the cells. In some embodiments, the subject has cancer (for example, a solid cancer or a hematological cancer such as ALL or CLL). In some embodiments, the subject has CLL. In embodiments, the subject has ALL. In embodiments, the subject has a solid cancer, for example, a solid cancer described herein. Exemplary GITR agonists include, for example, GITR fusion proteins and anti-GITR antibodies (for example, bivalent anti-GITR antibodies) such as, for example, a GITR fusion protein described in U.S. Pat. No. 6,111,090, European Patent No.: 090505B1, U.S. Pat. No. 8,586,023, PCT Publication Nos.: WO 2010/003118 and 2011/090754, or an anti-GITR antibody described, for example, in U.S. Pat. No. 7,025,962, European Patent No.: 1947183B1, U.S. Pat. Nos. 7,812,135, 8,388,967, 8,591,886, European Patent No.: EP 1866339, PCT Publication No.: WO 2011/028683, PCT Publication No.: WO 2013/039954, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/115451, U.S. Pat. No. 7,618,632, and PCT Publication No.: WO 2011/051726.

In some embodiments, a CAR expressing cell described herein is administered to a subject in combination with a GITR agonist, for example, a GITR agonist described herein. In some embodiments, the GITR agonist is administered prior to the CAR-expressing cell. For example, in some embodiments, the GITR agonist can be administered prior to apheresis of the cells. In some embodiments, the subject has CLL.

The methods described herein can further include formulating a CAR-expressing cell in a pharmaceutical composition. Pharmaceutical compositions may comprise a CAR-expressing cell, for example, a plurality of CAR-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (for example, aluminum hydroxide); and preservatives. Compositions can be formulated, for example, for intravenous administration.

In some embodiments, the pharmaceutical composition is substantially free of, for example, there are no detectable levels of a contaminant, for example, selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In some embodiments, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-cancer effective amount,” “a cancer-inhibiting effective amount,” or “therapeutic amount” is indicated, the precise amount of the compositions to be administered can be determined by a physician with consideration of individual differences in age, weight, tumor size, extent of infection or metastasis, and condition of the patient (subject). It can generally be stated that a pharmaceutical composition comprising the immune effector cells (for example, T cells, NK cells) described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, for example, Rosenberg et al., New Eng. J. of Med. 319:1676, 1988).

In some embodiments, a dose of CAR cells (for example, CD19 CAR cells) comprises about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (for example, CD19 CAR cells) comprises at least about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (for example, CD19 CAR cells) comprises up to about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (for example, CD19 CAR cells) comprises about 1.1×10⁶-1.8×10⁷ cells/kg. In some embodiments, a dose of CAR cells (for example, CD19 CAR cells) comprises about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (for example, CD19 CAR cells) comprises at least about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (for example, CD19 CAR cells) comprises up to about 1×10⁷, 2× 10⁷, 5×10⁷, 1×10⁸, 2×10⁸, 5×10⁸, 1×10⁹, 2×10⁹, or 5×10⁹ cells.

In some embodiments, it may be desired to administer activated immune effector cells (for example, T cells, NK cells) to a subject and then subsequently redraw blood (or have an apheresis performed), activate immune effector cells (for example, T cells, NK cells) therefrom, and reinfuse the patient with these activated and expanded immune effector cells (for example, T cells, NK cells). This process can be carried out multiple times every few weeks. In some embodiments, immune effector cells (for example, T cells, NK cells) can be activated from blood draws of from 10 cc to 400 cc. In some embodiments, immune effector cells (for example, T cells, NK cells) are activated from blood draws of 20 cc, 30 cc, 40 cc, 50 cc, 60 cc, 70 cc, 80 cc, 90 cc, or 100 cc.

The administration of the subject compositions may be carried out in any convenient manner. The compositions described herein may be administered to a patient trans arterially, subcutaneously, intradermally, intratumorally, intranodally, intramedullary, intramuscularly, by intravenous (i.v.) injection, or intraperitoneally, for example, by intradermal or subcutaneous injection. The compositions of immune effector cells (for example, T cells, NK cells) may be injected directly into a tumor, lymph node, or site of infection.

### Dosage Regimen

In some embodiments, a dose of viable CAR-expressing cells (for example, viable CD19, BCMA, CD20, or CD22 CAR-expressing cells) comprises about 0.5×10⁶ viable CAR-expressing cells to about 1.25×10⁹ viable CAR-expressing cells (for example, 0.5×10⁶ viable CAR-expressing cells to 1.25×10⁹ viable CAR-expressing cells). In some embodiments, a dose of viable CAR-expressing cells (for example, viable CD19, BCMA, CD20, or CD22 CAR-expressing cells) comprises about 1×10⁶, about 2.5×10⁶, about 5×10⁶, about 1.25×10⁷, about 2.5×10⁷, about 5×10⁷, about 5.75×10⁷, or about 8×10⁷ viable CAR-expressing cells.

### Patient Selection

In some embodiments of any of the methods of treating a subject, or composition for use disclosed herein, the subject has a cancer, for example, a hematological cancer. In some embodiments, the cancer is chosen from lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+large B-cell lymphoma, large B-cell lymphoma arising in HHV8-associated multicentric Castleman disease, primary effusion lymphoma, B-cell lymphoma, acute myeloid leukemia (AML), or unclassifiable lymphoma. In some embodiments, the cancer is a relapsed and/or refractory cancer.

In some embodiments of any of the methods of treating a subject, or composition for use disclosed herein, the subject has CLL or SLL. In some embodiments, the subject having CLL or SLL has previously been administered a BTK inhibitor therapy, for example, ibrutinib, for least 1-12 months, for example, 6 months. In some embodiments, the BTK inhibitor therapy, for example, ibrutinib therapy, is a second line therapy. In some embodiments, the subject had a partial response, or had stable disease in response to the BTK inhibitor therapy. In some embodiments, the subject did not response to the BTK inhibitor therapy. In some embodiments, the subject developed resistance, for example, developed ibrutinib resistance mutations. In some embodiments, the ibrutinib resistance mutations comprise a mutation in the gene encoding BTK and/or the gene encoding PLCg2. In some embodiments, the subject is an adult, for example, at least 18 years of age.

In some embodiments of any of the methods of treating a subject, or composition for use disclosed herein, the subject has DLBCL, for example, relapsed and/or refractory DLBCL. In some embodiments, the subject having DLBCL, for example, relapsed and/or refractory DLBCL, has previously been administered at least 2 lines of chemotherapy, for example, an anti-CD20 therapy and/or an anthracycline-based chemotherapy. In some embodiments, the subject has previously received stem cell therapy, for example, autologous stem cell therapy, and has not responded to said stem cell therapy. In some embodiments, the subject is not eligible for stem cell therapy, for example, autologous stem cell therapy. In some embodiments, the subject is an adult, for example, at least 18 years of age.

### Biomarkers for Evaluating CAR-Effectiveness

In some embodiments, disclosed herein is a method of evaluating or monitoring the effectiveness of a CAR-expressing cell therapy (for example, a CD19 or BCMA CAR therapy), in a subject (for example, a subject having a cancer, for example, a hematological cancer). The method includes acquiring a value of effectiveness to the CAR therapy, wherein said value is indicative of the effectiveness or suitability of the CAR-expressing cell therapy.

In embodiments, the value of effectiveness to the CAR therapy in a subject having CLL or SLL, comprises a measure of one, two, three, or all of the following parameters:
(i) a mutation in a gene encoding BTK in a sample (for example, an apheresis sample or a manufactured CAR-expressing cell product sample);
(ii) a mutation in a gene encoding PLCg2 in a sample (for example, an apheresis sample or a manufactured CAR-expressing cell product sample);
(iii) minimal residual disease, for example, as evaluated by the level and/or activity of CD8, CD4, CD3, CD5, CD19, CD20, CD22, CD43, CD79b, CD27, CD45RO, CD45RA, CCR7, CD95, Lag3, PD-1, Tim-3, and/or CD81; or as evaluated by immunoglobulin deep sequencing; in a sample (for example, an apheresis sample or tumor sample from the subject); or
(iv) the level or activity of one, two, three, four, five, six, seven, eight, nine, ten or all of the cytokines chosen from IFN-g, IL-2, IL-4, IL-6, IL-8, IL-10, IL-15, TNF-a, IP-10, MCP1, MIP1a, in a sample, for example, an apheresis sample from the subject.

In embodiments, the value of effectiveness to the CAR therapy in a subject having DLBCL, for example, relapsed and/or refractory DLBCL, comprises a measure of one or both the following parameters:
(i) minimal residual disease, for example, as evaluated by the level and/or activity of CD8, CD4, CD19, CD3, CD27, CD45RO, CD45RA, CCR7, CD95, Lag3, PD-1, and/or Tim-3; or as evaluated by immunoglobulin deep sequencing; in a sample (for example, an apheresis sample or tumor sample from the subject); or
(ii) the level or activity of one, two, three, four, five, six, seven, eight, nine, ten or all of the cytokines chosen from IFN-g, IL-2, IL-4, IL-6, IL-8, IL-10, IL-15, TNF-a, IP-10, MCP1, MIP1a, in a sample (for example, an apheresis sample from the subject).

In other embodiments, the value of effectiveness to the CAR therapy, further comprises a measure of one, two, three, four, five, six or more (all) of the following parameters:
(i) the level or activity of one, two, three, or more (for example, all) of resting TEFF cells, resting TREG cells, younger T cells (for example, naïve T cells (for example, naïve CD4 or CD8 T cells, naïve gamma/delta T cells), or stem memory T cells (for example, stem memory CD4 or CD8 T cells, or stem memory gamma/delta T cells), or early memory T cells, or a combination thereof, in a sample (for example, an apheresis sample or a manufactured CAR-expressing cell product sample);
(ii) the level or activity of one, two, three, or more (for example, all) of activated TEFF cells, activated TREG cells, older T cells (for example, older CD4 or CD8 cells), or late memory T cells, or a combination thereof, in a sample (for example, an apheresis sample or a manufactured CAR-expressing cell product sample);
(iii) the level or activity of an immune cell exhaustion marker, for example, one, two or more immune checkpoint inhibitors (for example, PD-1, PD-L1, TIM-3, TIGIT and/or LAG-3) in a sample (for example, an apheresis sample or a manufactured CAR-expressing cell product sample). In some embodiments, an immune cell has an exhausted phenotype, for example, co-expresses at least two exhaustion markers, for example, co-expresses PD-1 and TIM-3. In other embodiments, an immune cell has an exhausted phenotype, for example, co-expresses at least two exhaustion markers, for example, co-expresses PD-1 and LAG-3;
(iv) the level or activity of CD27 and/or CD45RO− (for example, CD27+CD45RO−) immune effector cells, for example, in a CD4+ or a CD8+ T cell population, in a sample (for example, an apheresis sample or a manufactured CAR-expressing cell product sample);
(v) the level or activity of one, two, three, four, five, six, seven, eight, nine, ten, eleven or all of the biomarkers chosen from CCL20, IL-17a, IL-6, PD-1, PD-L1, LAG-3, TIM-3, CD57, CD27, CD122, CD62L, KLRG1;
(vi) a cytokine level or activity (for example, quality of cytokine reportoire) in a CAR-expressing cell product sample, for example, CLL-1-expressing cell product sample; or
(vii) a transduction efficiency of a CAR-expressing cell in a manufactured CAR-expressing cell product sample.

In some embodiments of any of the methods disclosed herein, the CAR-expressing cell therapy comprises a plurality (for example, a population) of CAR-expressing immune effector cells, for example, a plurality (for example, a population) of T cells or NK cells, or a combination thereof. In some embodiments, the CAR-expressing cell therapy is a CD19 CAR therapy.

In some embodiments of any of the methods disclosed herein, the measure of one or more of the parameters disclosed herein is obtained from an apheresis sample acquired from the subject. The apheresis sample can be evaluated prior to infusion or re-infusion.

In some embodiments of any of the methods disclosed herein, the measure of one or more of the parameters disclosed herein is obtained from a tumor sample acquired from the subject.

In some embodiments of any of the methods disclosed herein, the measure of one or more of the parameters disclosed herein is obtained from a manufactured CAR-expressing cell product sample, for example, CD19 CAR-expressing cell product sample. The manufactured CAR-expressing cell product can be evaluated prior to infusion or re-infusion.

In some embodiments of any of the methods disclosed herein, the subject is evaluated prior to receiving, during, or after receiving, the CAR-expressing cell therapy.

In some embodiments of any of the methods disclosed herein, the measure of one or more of the parameters disclosed herein evaluates a profile for one or more of gene expression, flow cytometry or protein expression.

In some embodiments of any of the methods disclosed herein, the method further comprises identifying the subject as a responder, a non-responder, a relapser or a non-relapser, based on a measure of one or more of the parameters disclosed herein.

In some embodiments of any of the methods disclosed herein, a responder, for example, complete responder has, or is identified as having, a greater, for example, a statistically significant greater, percentage of CD8+ T cells compared to a reference value, for example, a non-responder percentage of CD8+ T cells.

In some embodiments of any of the methods disclosed herein, a responder, for example, complete responder has, or is identified as having, a greater percentage of CD27+CD45RO− immune effector cells, for example, in the CD8+ population, compared to a reference value, for example, a non-responder number of CD27+CD45RO-immune effector cells.

In some embodiments of any of the methods disclosed herein, a responder, for example, complete responder or a partial responder has, or is identified as having, a greater, for example, a statistically significant greater, percentage of CD4+ T cells compared to a reference value, for example, a non-responder percentage of CD4+ T cells.

In some embodiments of any of the methods disclosed herein, a responder, for example, complete responder has, or is identified as having, a greater percentage of one, two, three, or more (for example, all) of resting TEFF cells, resting TREG cells, younger T cells, or early memory T cells, or a combination thereof, compared to a reference value, for example, a non-responder number of resting TEFF cells, resting TREG cells, younger T cells, or early memory T cells.

In some embodiments of any of the methods disclosed herein, a non-responder has, or is identified as having, a greater percentage of one, two, three, or more (for example, all) of activated TEFF cells, activated TREG cells, older T cells (for example, older CD4 or CD8 cells), or late memory T cells, or a combination thereof, compared to a reference value, for example, a responder number of activated TEFF cells, activated TREG cells, older T cells (for example, older CD4 or CD8 cells), or late memory T cells.

In some embodiments of any of the methods disclosed herein, a non-responder has, or is identified as having, a greater percentage of an immune cell exhaustion marker, for example, one, two or more immune checkpoint inhibitors (for example, PD-1, PD-L1, TIM-3, TIGIT, and/or LAG-3). In some embodiments, a non-responder has, or is identified as having, a greater percentage of PD-1, PD-L1, or LAG-3 expressing immune effector cells (for example, CD4+ T cells and/or CD8+ T cells) (for example, CAR-expressing CD4+ cells and/or CD8+ T cells) compared to the percentage of PD-1 or LAG-3 expressing immune effector cells from a responder.

In some embodiments, a non-responder has, or is identified as having, a greater percentage of immune cells having an exhausted phenotype, for example, immune cells that co-express at least two exhaustion markers, for example, co-expresses PD-1, PD-L1 and/or TIM-3. In other embodiments, a non-responder has, or is identified as having, a greater percentage of immune cells having an exhausted phenotype, for example, immune cells that co-express at least two exhaustion markers, for example, co-expresses PD-1 and LAG-3.

In some embodiments of any of the methods disclosed herein, a non-responder has, or is identified as having, a greater percentage of PD-1/PD-L1+/LAG-3+ cells in the CAR-expressing cell population (for example, a CLL-1 CAR+ cell population) compared to a responder (for example, a complete responder) to the CAR-expressing cell therapy.

In some embodiments of any of the methods disclosed herein, the responder (for example, the complete or partial responder) has one, two, three or more (or all) of the following profile:
(i) has a greater number of CD27+ immune effector cells compared to a reference value, for example, a non-responder number of CD27+ immune effector cells;
(ii) has a greater number of CD8+ T cells compared to a reference value, for example, a non-responder number of CD8+ T cells;
(iii) has a lower number of immune cells expressing one or more checkpoint inhibitors, for example, a checkpoint inhibitor chosen from PD-1, PD-L1, LAG-3, TIM-3, or KLRG-1, or a combination, compared to a reference value, for example, a non-responder number of cells expressing one or more checkpoint inhibitors; or
(iv) has a greater number of one, two, three, four or more (all) of resting TEFF cells, resting TREG cells, naïve CD4 cells, unstimulated memory cells or early memory T cells, or a combination thereof, compared to a reference value, for example, a non-responder number of resting TEFF cells, resting TREG cells, naïve CD4 cells, unstimulated memory cells or early memory T cells.

In embodiments, a subject who is a responder, a non-responder, a relapser or a non-relapser identified by the methods herein can be further evaluated according to clinical criteria. For example, a complete responder has, or is identified as, a subject having a disease, for example, a cancer, who exhibits a complete response, for example, a complete remission, to a treatment. A complete response may be identified, for example, using the NCCN Guidelines®, or the International Workshop on Chronic Lymphocytic Leukemia (iwCLL) 2018 guidelines as disclosed in Hallek M et al., Blood (2018) 131:2745-2760 “iwCLL guidelines for diagnosis, indications for treatment, response assessment, and supportive management of CLL,” the entire contents of which are hereby incorporated by reference in its entirety. A partial responder has, or is identified as, a subject having a disease, for example, a cancer, who exhibits a partial response, for example, a partial remission, to a treatment. A partial response may be identified, for example, using the NCCN Guidelines®, or iwCLL 2018 criteria as described herein. A non-responder has, or is identified as, a subject having a disease, for example, a cancer, who does not exhibit a response to a treatment, for example, the patient has stable disease or progressive disease. A non-responder may be identified, for example, using the NCCN Guidelines®, or iwCLL 2018 criteria as described herein.

Alternatively, or in combination with the methods disclosed herein, responsive to said value, performing one, two, three four or more of:
administering for example, to a responder or a non-relapser, a CAR-expressing cell therapy;
administered an altered dosing of a CAR-expressing cell therapy;
altering the schedule or time course of a CAR-expressing cell therapy;
administering, for example, to a non-responder or a partial responder, an additional agent in combination with a CAR-expressing cell therapy, for example, a checkpoint inhibitor, for example, a checkpoint inhibitor described herein;
administering to a non-responder or partial responder a therapy that increases the number of younger T cells in the subject prior to treatment with a CAR-expressing cell therapy;
modifying a manufacturing process of a CAR-expressing cell therapy, for example, enriching for younger T cells prior to introducing a nucleic acid encoding a CAR, or increasing the transduction efficiency, for example, for a subject identified as a non-responder or a partial responder;
administering an alternative therapy, for example, for a non-responder or partial responder or relapser; or
if the subject is, or is identified as, a non-responder or a relapser, decreasing the TREG cell population and/or TREG gene signature, for example, by one or more of CD25 depletion, administration of cyclophosphamide, anti-GITR antibody, or a combination thereof.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1: Generation of CARTs with Cytokine Stimulation

### Summary

This example describes a CART manufacturing process called “cytokine process.” In some embodiments, cells (for example, T cells) are seeded in media (for example, serum-containing media, for example, media containing 2% serum). One or more cytokines (for example, one or more cytokines chosen from IL-2, IL-7, IL-15 (for example, hetIL-15 (IL15/sIL-15Ra)), IL-21, or IL-6 (for example, IL-6/sIL-6Ra) as well as vectors (for example, lentiviral vectors) encoding a CAR are added to the cells. After incubation for 20-24 hours, cells are washed, formulated, and cryopreserved. Exemplary cytokine process is shown in FIG. 1A.

Compared to the traditional CART manufacturing process, this revised process eliminates CD3/CD28 stimulation as well as ex vivo T cell expansion. Without wishing to be bound by theory, anti-CD3/anti-CD28 beads drive differentiation into central memory cells; and in contrast, cytokines such as IL-15, IL-21, and IL-7 may help preserve the undifferentiated phenotype of transduced CD3+ T cells. As a consequence, the cytokine process which does not involve CD3/CD28 activation may generate CART cells with a higher percentage of naïve/stem T cells, compared to CART cells generated using the traditional approach.

### Methods

After obtaining an apheresis within 24 hours of collection, T cells were purified and the purity of the T-cells obtained was assessed by flow cytometry. The T cells were frozen and placed in the liquid nitrogen until required for use.

Alternatively, a cryopreserved apheresis sample is prepared and enriched for CD4+ T cells and/or CD8+ T cells using a Prodigy® machine.

IL-7 and IL-15 were prepared at 1,000 folds of the final concentration required. IL-2 was prepared by a 10-fold dilution in media.

TABLE 19
Cytokine conditions

| | Conditions |
|---|---|
| 1. | IL2 |
| 2. | IL-7 |
| 3. | IL-15 |
| 4. | IL2 + IL7 |
| 5. | IL-7 + IL-15 |
| 6. | IL2 + IL-15 |
| 7. | Beads + IL2 |
| 8. | Beads + IL15 |

In the expander bead stimulated conditions, calculations were performed to plate cells with a final concentration of bead to cell ratio of 3:1. The Dynabeads® magnetic beads were washed twice using a Dynamag® and resuspended in the required volume of media for the experiment. The washed beads were added to the tubes that contained the specific cytokines and cells.

At the time of plating, the cells were transduced with a lentiviral vector with a multiplicity of infection (MOI) of 1. The specific volume of vector to be transduced was calculated based on the multiplicity of infection (MOI) and concentration (titer) of the vector lot in use. The titer and the MOI were measured based on primary T cell lines.

In the conditions where cytokines alone were utilized for stimulation, the cells were resuspended post wash at a concentration of 1E7/ml and added to a conical tube that already contained the cytokines depending on the condition (Table 19). After the cells and cytokines were added the lentiviral vector was added followed by the media.

In all of the conditions the cells were mixed and 1 ml was plated in 14 wells of a 24 well plate. The cells were placed in an incubator that was at 37° C. and 5% CO₂.

On the following day the cells were harvested, the concentration and viability of the cells was noted. Their function was measured using a cytotoxicity and proliferation (EDU) incorporation assay. These cells were referred to as “day 1 CARTs.”

The cells were immunophenotyped for T cell differentiation status and transduction of the CAR was assessed using flow cytometry. The cells were washed, viability dye was added followed by the antibody cocktail (Table 20), and the plates were incubated for 20 minutes at room temperature. After the incubation, the cells were washed twice and fixed prior to being analyzed on the BD fortessa.

TABLE 20
Antigens of the panel of antibodies used to determine the
differentiation status of the T-cells
Antigen
Viability
CD3
CD4
CD8
HLADR
CD28
CD45RO
CD95
CCR7
Anti-Idiotype

To determine if the day 1 CARTs still maintained the ability to expand post-harvest, 5e6 cells/condition were expanded using CD3/CD28 beads in a T25 flask at a ratio of 3:1 (beads to cells). The Dynabeads® magnetic beads were washed as previously described. The media contained no cytokines. The cells were placed in an incubator that was at 37° C. and 5% CO₂.

In the case of the T cells expanded with the CD3/CD28 beads every 2 days, the cells were counted and spilt up to 10 days in culture. On day 10 the cells were harvested, counted, immunophenotyped using the differentiation panel (Table 20) and frozen in Cryostor 10™. The cells were thawed for functional assays that included cytotoxicity assay, proliferation assay and cytokine secretion assay.

The cells expanded in the presence of CD3/CD28 beads in vitro for 10 days were referred to as “day 10 CARTs.”

### Results

When purified T cells were incubated with cytokines in the absence of any other activation stimulus, there was an increase in transduction from day 1 to day 4 (FIG. 1B). Independent of the time point and cytokine condition, the predominant population within the CAR positive population was naïve (FIGS. 1D, 1E, and 1F). The elimination of the activation agent led to an enhancement of transduction with the primitive population. Notably, exposure to IL-2 or IL-15 maintained self-renewing T cells in vitro (FIG. 1G). Similar phenomenon was observed under the other cytokine treatments tested (IL-7; IL2+IL7; IL-7+IL-15; and IL2+IL-15) (data not shown). The cytokine process (using IL2 or IL-15 in this specific example) maintained or slightly increased the percentage of CD45RO− CCR7+ cells (FIG. 1G). Similar data are shown in FIGS. 1H and 1I for IL-2, IL-15, and a combination of IL-7 and IL-15. Culturing T cells with the indicated cytokines for 24 hours maintained the naïve phenotype of CD3+ T cells, and reduced the percentage of central memory T cells (FIGS. 1H and 1I).

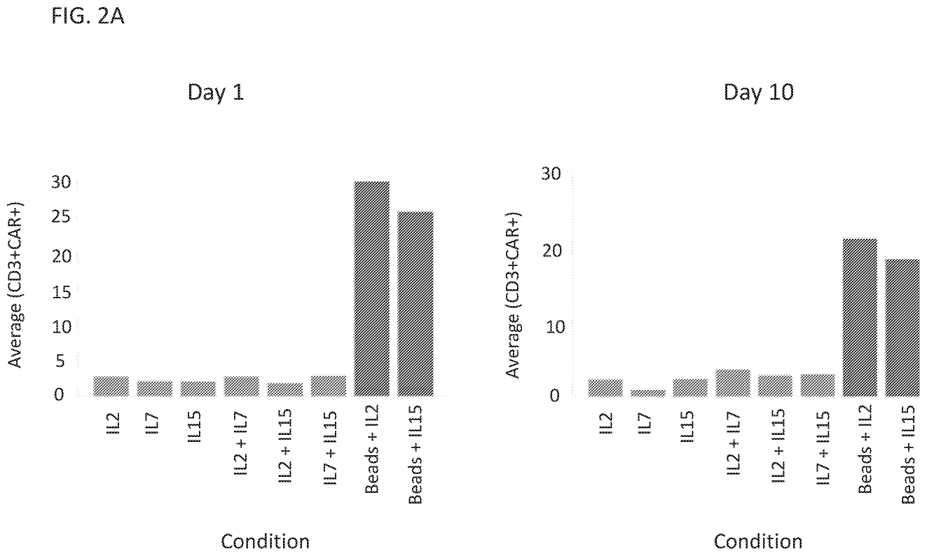

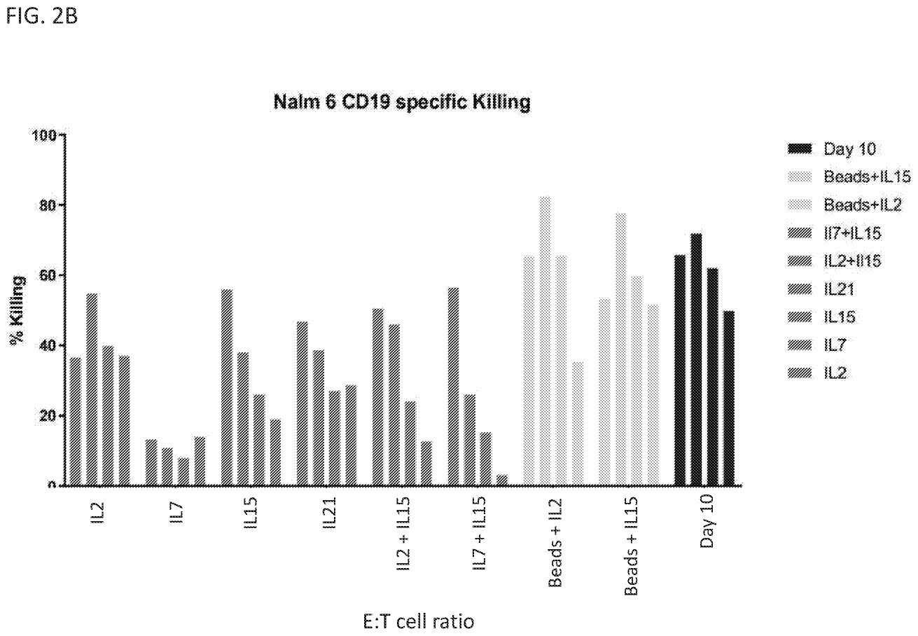

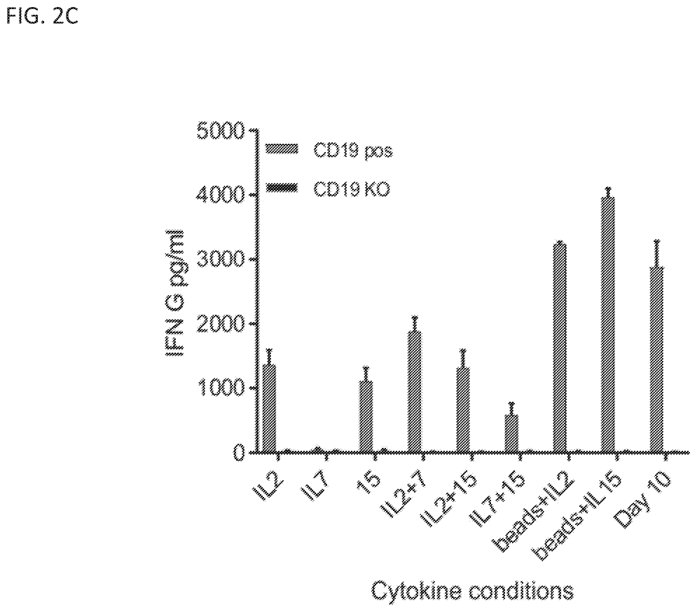

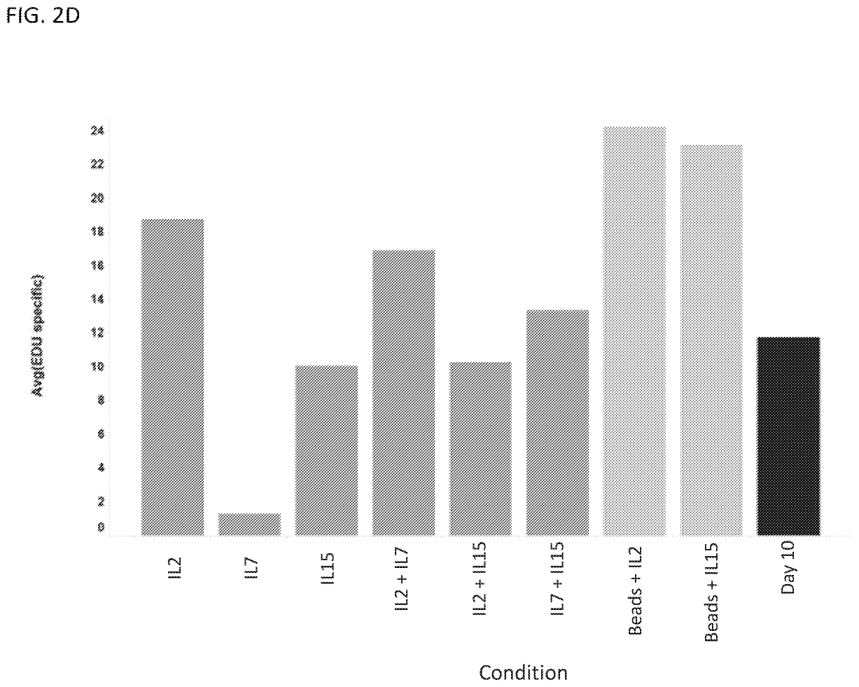

To ensure that the transduction observed within 24 hours was stable, the CARTs generated within 24 hours were washed to remove any residual virus and expanded over 10 days using CD3/D28 expansion beads. The expanded cells demonstrated almost equivalent transduction to the day 1 CARTs indicating that the transduction was stable (FIG. 2A).

The functionality of the day 1 CARTs and day 10 CARTs was tested using a cytotoxicity, a cytokine release, and a proliferation assay. The target cells were Nalm6 cells, a B cell ALL cell line that expresses CD19. The cytotoxicity assay demonstrated that the day 1 CARTs post expansion were equivalent at killing as compared to the day 10 CARTs (FIG. 2B) even though the day 1 CARTs had much fewer transduced cells. The same day 1 CARTs that had been expanded were compared for the secretion for IFN-gamma and found to have a lower secretion of IFN-gamma as compared to the day 10 CARTs (FIG. 2C), which was likely due to the difference in the number of transduced cells. In separate studies where the day 1 CARTs had a higher level of transduction, they secreted a higher level of IFN-gamma (data not shown). Furthermore, the day 1 CARTs from all the treatment conditions except the IL7-only condition showed similar or higher proliferation than the day 10 CARTs (FIG. 2D). The data shown in FIG. 2D were not normalized for transduction levels.

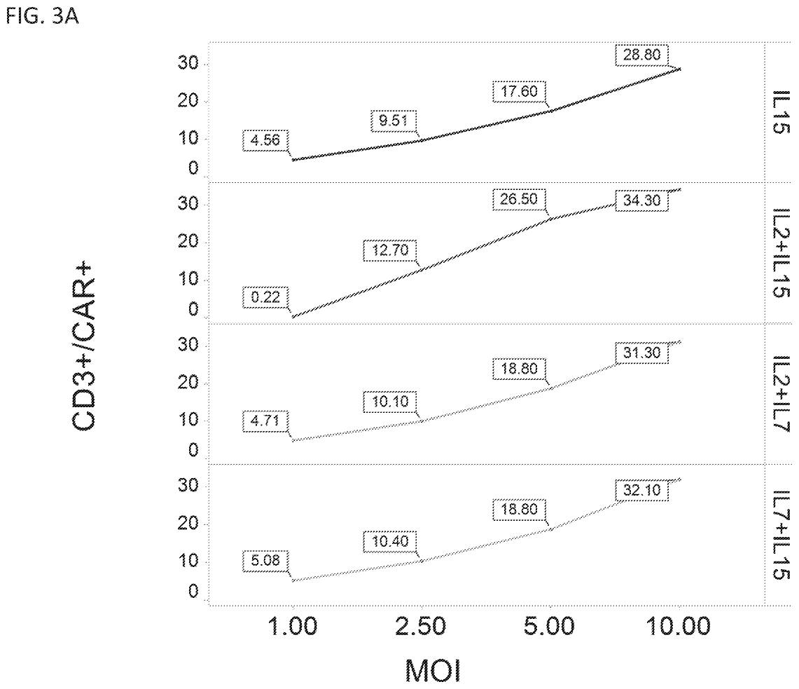

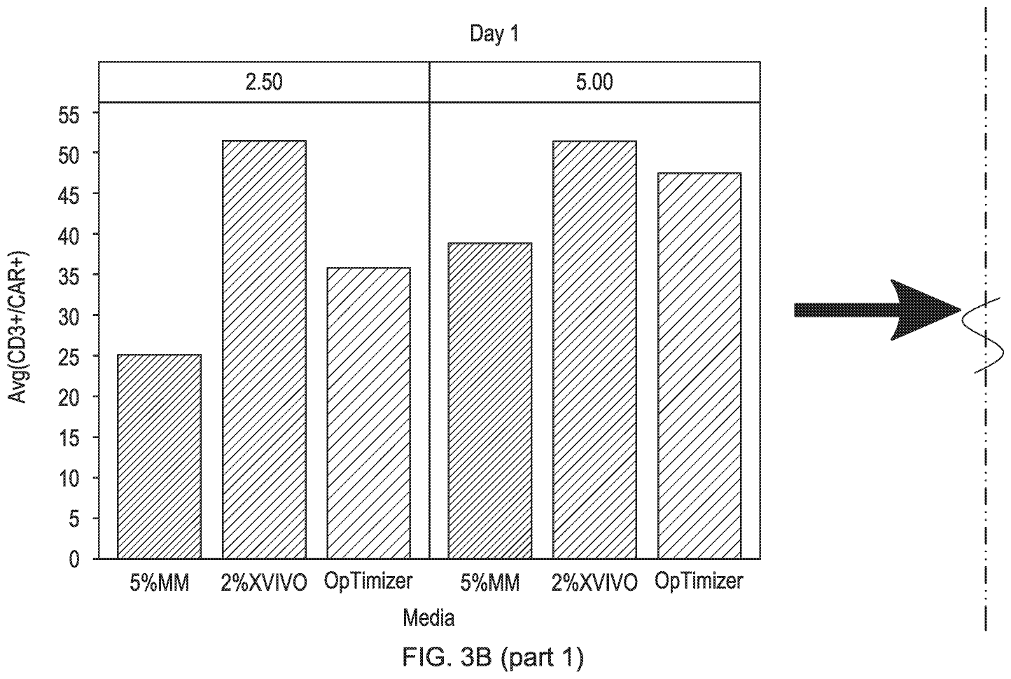

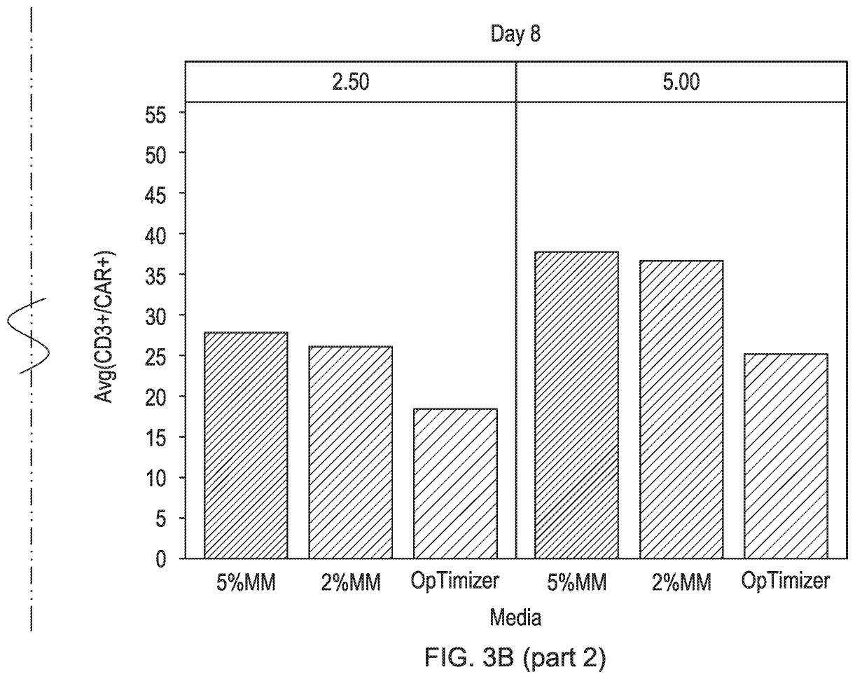

Although stable transduction was observed in the day 10 CARTs, the efficiency was consistently low. A titration of increasing multiplicity of infection (MOI) of the lentiviral vector was tested in four cytokine conditions and in all conditions tested a linear relationship with transduction was observed (FIG. 3A).

Furthermore, different media compositions (mainly a reduction in serum concentration from 5% to 2% to serum free) were compared to determine whether they impact the transduction efficiency. The reduction in serum to 2% human serum led to the highest transduction efficiency (FIG. 3B). The addition of Glutamax alone was also considered to have a significant impact on transduction efficiency.

Next, the day 1 CARTs and day 10 CARTs were examined for their anti-tumor activity in vivo using a mouse ALL model. Briefly, day 1 CARTs and day 10 CARTs were manufactured as described above with a viability above 80% (FIGS. 4A and 4B). CARTs were administered in tumor-bearing mice and monitored for expansion in vivo. As shown in FIG. 4C, day 1 CARTs showed a higher level of in vivo expansion than their day 10 counterparts. In particular, CARTs manufactured in the presence of IL-2 showed the highest level of in vivo expansion (FIG. 4C). All the CARTs tested inhibited tumor growth in vivo, although day 1 CARTs showed a delayed kinetics as compared to the day 10 CARTs (FIG. 4D). In this specific donor, the IL2 condition demonstrated the greatest ability to eliminate the tumor in vivo (FIG. 4D).

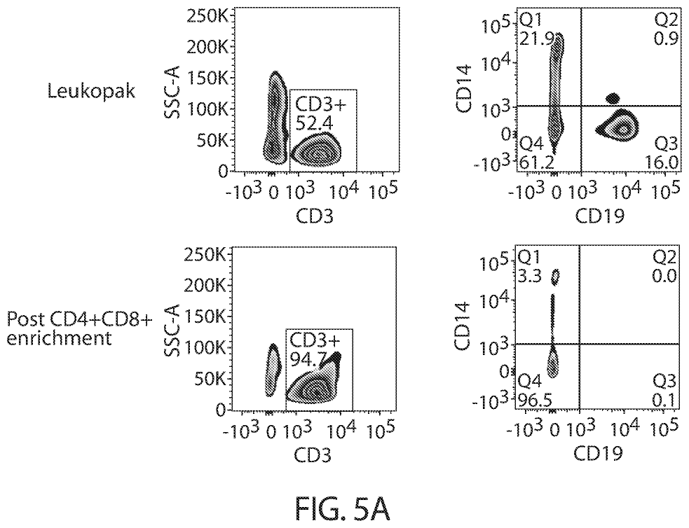

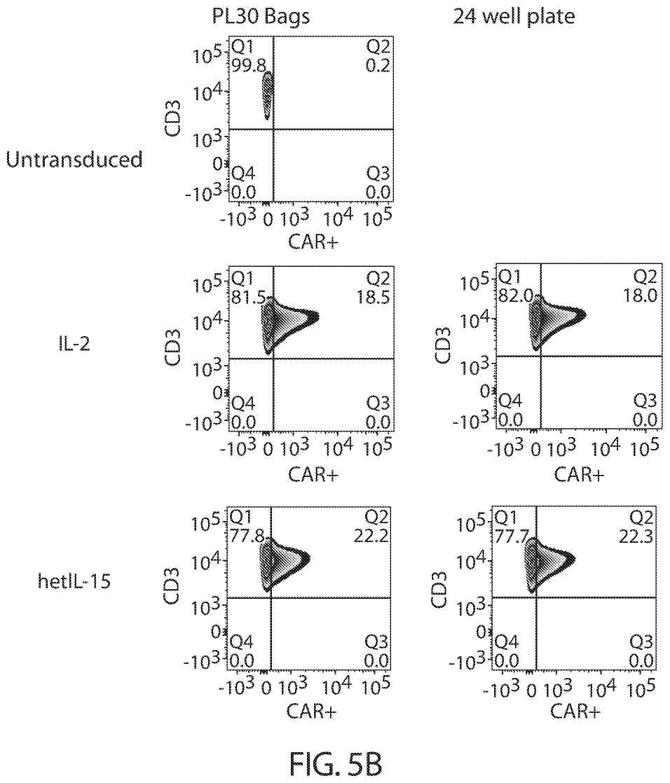

Furthermore, it was tested whether this manufacturing process was scalable. Purified T cells from a frozen apheresis sample were transduced with an anti-CD19 CAR in either a 24 well plate or a PL30 bag post enrichment, in the presence of either IL2 or hetIL-15 (IL15/sIL-15Ra). hetIL-15 has been described in WO 2014/066527, herein incorporated by reference in its entirety, and comprises human IL-15 complexed with a soluble form of human IL-15Ra. Cells were harvested 24 hours later and tested for expression of CAR. As shown in FIG. 5B, there was no impact on transduction observed when the process was scaled from a 24 well plate to a PL30 bag in the presence of either IL2 or hetIL-15.

### Example 2: Generation of CARTs with TCR Stimulation

### Summary

This example describes a CART manufacturing process called “activation process.” In some embodiments, cells (for example, T cells) are seeded in media (for example, serum-free media, for example, OpTmizer™ media) containing IL-2 (for example, OpTmizer™ media containing OpTmizer™ supplement, GlutaMAX and 100 IU/ml of IL-2), placed in a cell culture device, and contacted with anti-CD3/anti-CD28 (for example, TransAct). After 12 hours, a vector (for example, a lentiviral vector) encoding a CAR is added to the cells and the cells are returned to an incubator. At 24 hours from initiation of the cell culture, the cells are harvested, sampled, and formulated. Without wishing to be bound by theory, brief CD3 and CD28 activation, for example, using anti-CD3/anti-CD28 (for example, TransAct), promotes efficient transduction of self-renewing T cells.

In this and other examples, a CART manufacturing process called “traditional manufacturing (TM)” process was used as a control. In some embodiments, T cells are selected from a fresh or cryopreserved leukapheresis sample (for example, using positive or negative selection), activated (for example, using anti-CD3/anti-CD28 antibody coated Dynabeads®), contacted with a nucleic acid molecule encoding a CAR molecule (for example, transduced with a lentiviral vector comprising a nucleic acid molecule encoding the CAR molecule), and expanded in vitro for, for example, 7, 8, 9, 10, or 11 days. An exemplary TM process is provided in this example as the methods used to manufacture CAR cells from the d9 control arms.

### Methods

In some embodiments, the activation process provided herein starts with a frozen or fresh leukapheresis product. After a sample for counting and QC is obtained, the product is attached to a cell sorting machine (for example, an installed CliniMACS® Prodigy® device kit) and the program begins. The cells are washed and incubated with microbeads that bind to desired surface marker or markers (such as CD3, CD4, CD8, CD27, CD28, CD45RO, CCR7, CD62L, CD14, CD34, CD95, CD19, CD20, CD22, and/or CD56). The bead-labeled cells are selected by passing the cells through a magnetic column. If desired, cells can be further separated by incubating the negative fraction with beads that bind to a second set of surface markers (such as CD3, CD4, CD8, CD27, CD28, CD45RO, CCR7, CD62L, CD14, CD34, CD95, CD19, CD20, CD22, and/or CD56) and again passing the cells through a magnetic separation column. Isolated cells are washed again and the separation buffer is exchanged for cell media. Purified cells then either proceed to culture or are cryopreserved for later use. Cryopreserved cells can be thawed, washed in pre-warmed cell media, and resuspended in cell media. Fresh cells can be added to culture directly. The cells are seeded into membrane bioreactors at 0.4-1.2e6 cells/cm² of membrane, an activating reagent such as anti-CD3/anti-CD28 beads/polymers, nanoparticles, or nanocolloids (and/or any of the following co-activators alone or in combination: a reagent that stimulates ICOS, CD27, HVEM, LIGHT, CD40, 4-1BB, OX40, DR3, GITR, CD30, TIM1, CD2, or CD226) is added, and cell media is added to a final volume of 0.25-2 ml/cm² of membrane. A vector (for example, a lentiviral vector) encoding the CAR is added immediately or up to 18 hours after culture initiation. The cells are incubated with the vector and the activating reagent described above for a total of 24 hours post culture initiation. Once culture has proceeded for 24 hours, the cells are resuspended mechanically by swirling or pipetting or otherwise agitating, and simulating reagent scaffolds are dissolved with appropriate buffers. The cells are washed to remove unnecessary reagents and reformulated in cryopreservation media. The cells are cryopreserved until needed for administration.

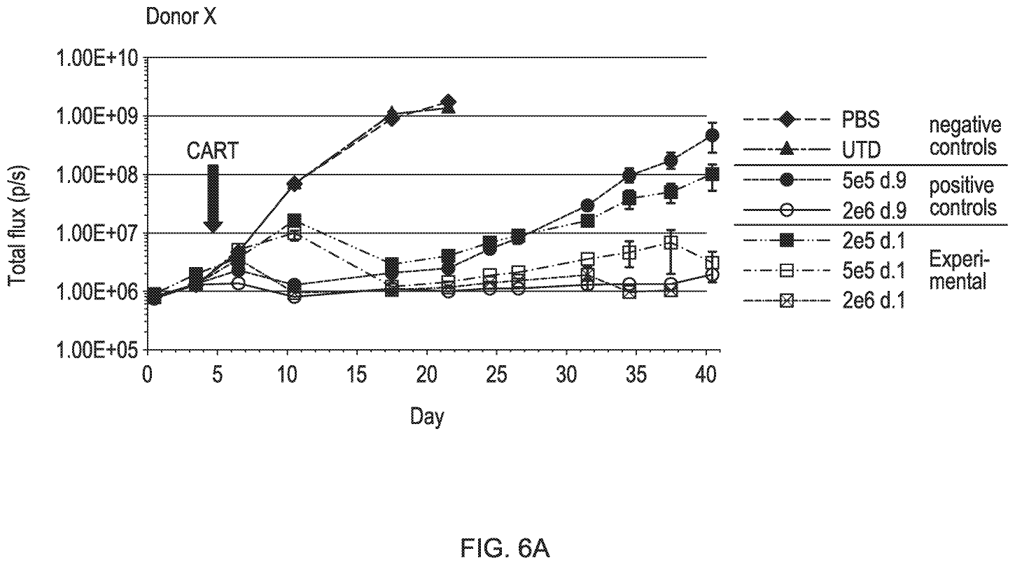

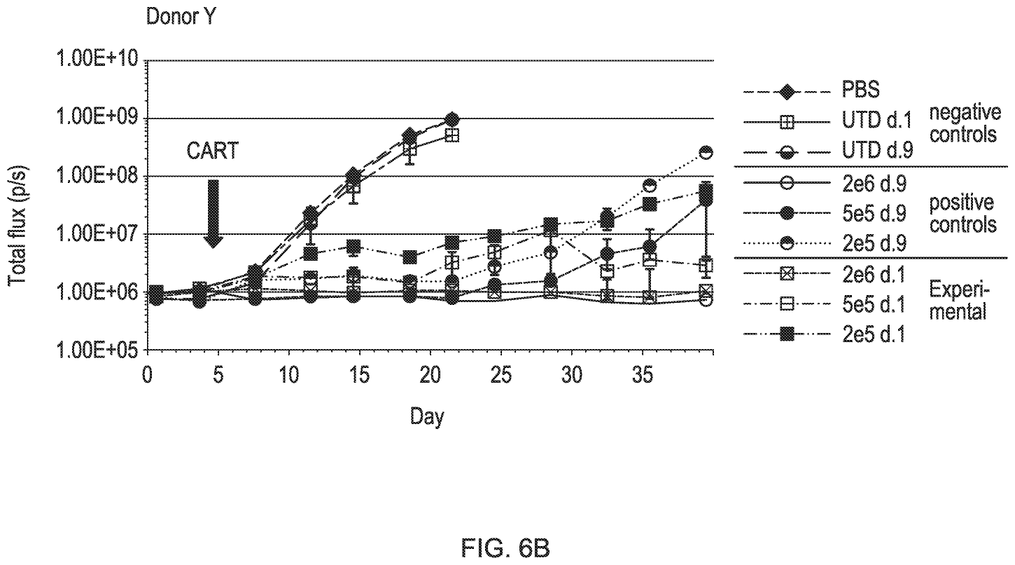

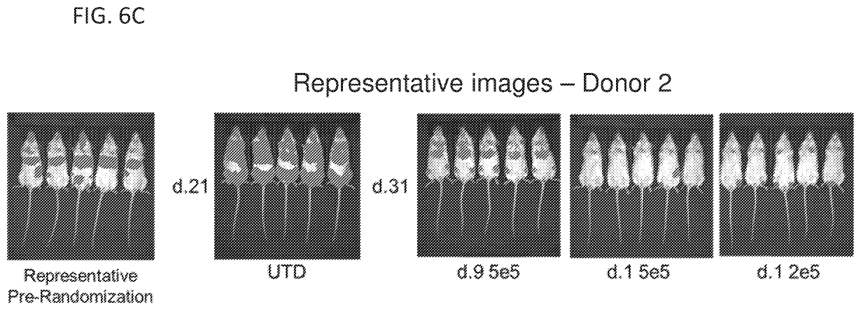

For studies related to FIGS. 6A-6C, the following protocol was used.

Cells were purified from a fresh ¼ leukopack using automated ficoll (Sepax 2, BioSafe) to generate peripheral blood mononuclear cells (PBMC). These PBMCs were further purified using immunomagnetic negative selection (PanT Negative Selection Kit, Miltenyi) to generate CD3 T-cells of high purity (98-100%). These cells were placed in culture with OpTmizer™ (Thermo) complete media (formulated per package insert and supplemented with IL-2 at 100 IU/ml (Proleukin, Prometheus)) and an anti-CD3/CD28 activation reagent at the recommended dose (TransAct, Milenyi) in a membrane bioreactor. Cells were then incubated at 37° C., 5% CO₂ for 12 hours for activation. Cells were removed from the incubator and freshly thawed lentiviral vector was added to the cultures at a multiplicity of infection (MOI) of 2.5 tu/cell. Cells were returned to the incubator for another 12 hours for transduction. Cells were harvested, washed twice with media, and formulated directly into sterile PBS (Invitrogen) and injected into NSG mice via the tail vein. Cells from the d9 control arms were grown in flasks (T25-T225, Corning) using RPMI media (Thermo) supplemented with 10% fetal bovine serum (Seradigm) (complete media a.k.a “R10”) and anti CD3/28 Expander Dynabeads® (Thermo) at 3 beads per T-cell. Cells were then incubated at 37° C., 5% CO₂ for 24 hours for activation. Cells were removed from the incubator and freshly thawed lentiviral vector was added to the cultures at a MOI of 2.5 tu/cell. Cells were returned to the incubator for an additional 7 days, splitting every 2 days to maintain a concentration of 5e5 cells/ml. Expanded cells were transferred to 50 ml centrifuge tubes (Corning) and subjected to two rounds of bead removal using a standing magnet (Dynamag-50, Thermo). Debeaded cells were then washed twice with media, and formulated into CryoStor10 cryomedia (STEMCELL Technologies), cryopreserved using a CoolCell device (BioCision), and kept in vapor phase liquid nitrogen for a minimum of 48 hours. Cells were thawed into prewarmed R10 media, washed twice with media, then formulated into sterile PBS (Invitrogen) and injected into NSG mice via the tail vein.

6-8 week old NSG mice (NOD.Cg-PrkdcscidIl2rgtm1Wjl/SzJl, Jackson Labs) were injected with luciferized NALM6 tumor cells (ATCC CRL-3273, ATCC) at 1e6 cells/mouse 4 day prior to CART injection without preconditioning. PBS formulated CART cells were injected at 2e6, 5e5, or 2e5 CAR+ cells per NSG or a matched dose of untransduced expanded T-cells or a PBS vehicle control. Mice were monitored by weekly blood draw, bi-weekly luciferase imaging (Xenogen IVIS, PerkinElmer), and bi-weekly weight measurements. All animals were monitored for signs of toxicity (weight loss, moribund) and euthanized if symptomatic. All surviving mice were euthanized at study termination (week 5) and terminal blood, bone marrow, and spleen samples were obtained. Study was performed according to IACUC and all other applicable guidelines.

### Results

CART cells were generated using the activation process described above and characterized for their in vivo anti-tumor activity in a mouse ALL model. As shown in FIGS. 6A-6C, CART cells manufactured using the activation process showed strong anti-tumor activity in vivo.

### Example 3: IL6R Expression on T Cells and Cytokine Effect on T Cell Expansion

### Material and Methods

### T Cell Culture

Previously frozen T cells were thawed and contacted with αCD3/αCD28 dyna1 beads (cell to bead ratio of 1 to 3) in the presence of indicated cytokines at day 0. From day 3, twice more T cell growth media (RPMI1640, 10% FBS, 2 mM L-glutamin, 100 μM non-essential amino acids, 1 mM sodium pyruvate, 10 mM Hepes, 55 μM β-mercaptoethanol, 10% FBS, and 100 U/ml of penicillin-streptomycin) was added to the plate with indicated cytokines (without cytokine, rhIL2 (50 IU/ml, Novartis), IL6 (10 ng/ml, R&D systems), IL7 (10 ng/ml, Peprotech), IL15 (10 ng/ml, Peprotech), and IL21 (10 ng/ml, Peprotech)) at day 3, 5, 6, 9, 12, 15, and 18. Cells treated without cytokine, IL6, or IL21 were cultured until day 18 and cells treated with IL2, IL7, or IL15 were cultured until day 25.

### Cell Surface Staining

Cells were harvested at indicated time points and then stained with live/dead dye (eFluro780, eBioscience), CD3 (BioLegend, clone #: OKT3), CD4 (BioLegend, clone #: OKT4), CD8 (BD Bioscience, clone #: RPA-T8), CD45RO (BioLegend, clone #: UCHL1), CCR7 (BioLegend, clone #: G043H7), CD27 (BD Horizon, clone #: L128), CD127 (BioLegend, clone #: A019D5), CD57 (BioLegend, clone #: HCD57), CD126 (BioLegend, clon #: UV4), and CD130 (R&D Systems, clone #: 28126) antibodies. The cells were acquired by FACS Fortessa and then FlowJo program was used for data analysis.

### Intracellular Cytokine Staining

To examine percent of cytokine producing cells, at day 25, T cells were harvested and then briefly activated with PMA (50 ng/ml, Sigma-Aldrich) and Ionomycin (1 μM, Sigma-Aldrich) for 4 hours in the presence of Brefeldin A (BioLegend) at 37° C. incubator. T cells were then stained with live/dead dye (eFluro780, eBioscience), CD3 (BioLegend, clone #: OKT3), CD4 (BioLegend, clone #: OKT4), CD8 (BD Bioscience, clone #: RPA-T8) antibodies followed by fixation and permeabilization. Then, T cells were further stained with antibodies against IFN-γ (BioLegend, clone #: 4S.B3), IL-2 (BioLegend, MQ1-17H12), and TNF-α (BioLegend, Mab11). The cells were acquired by FACS Fortessa and then FlowJo program was used for data analysis.

### Results

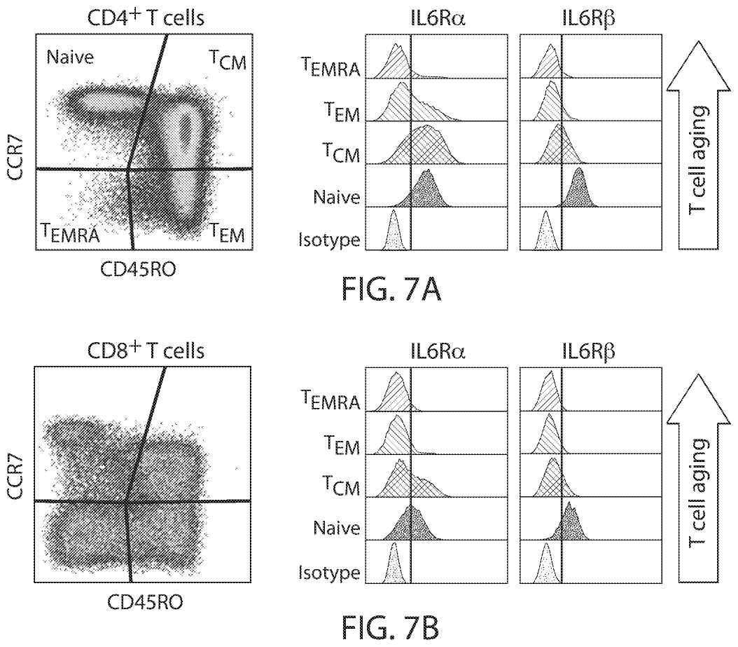

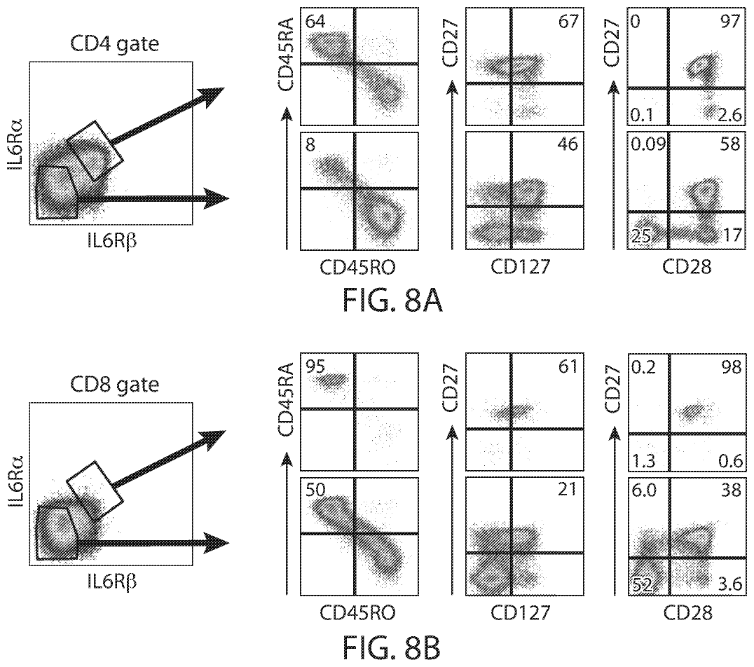

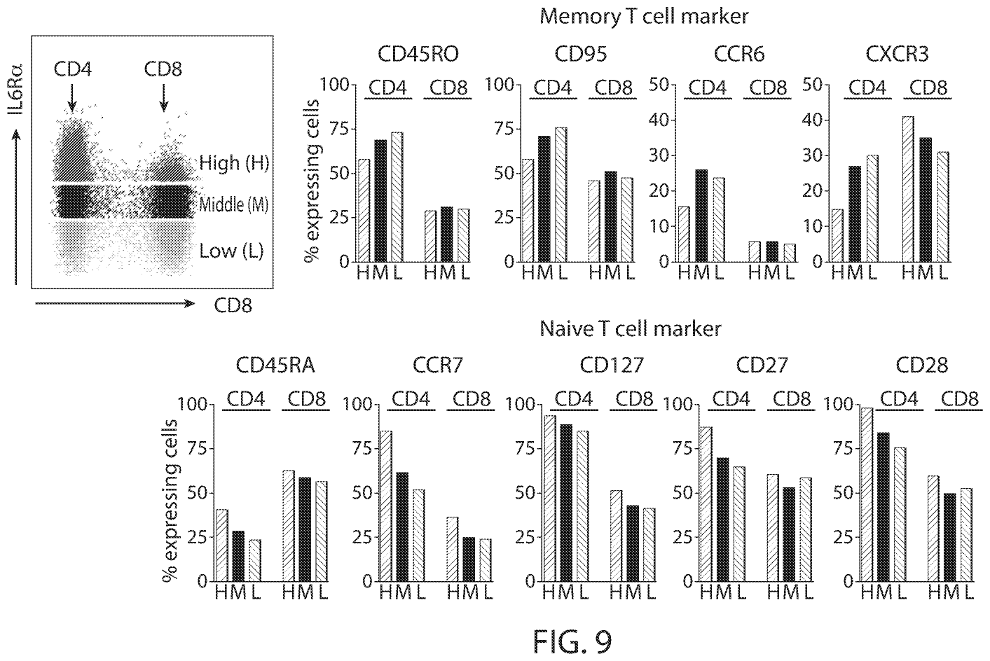

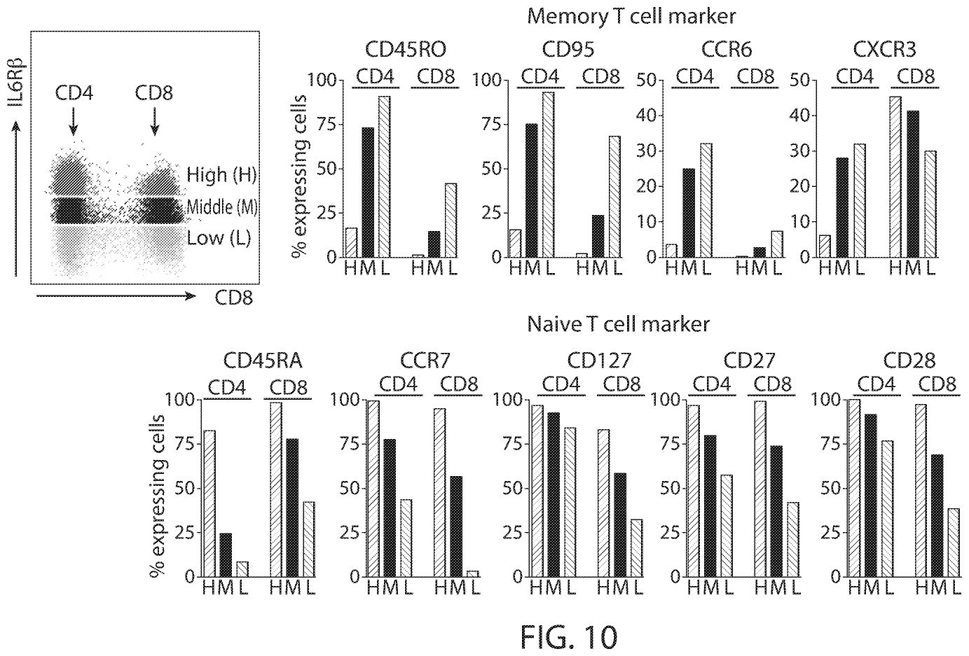

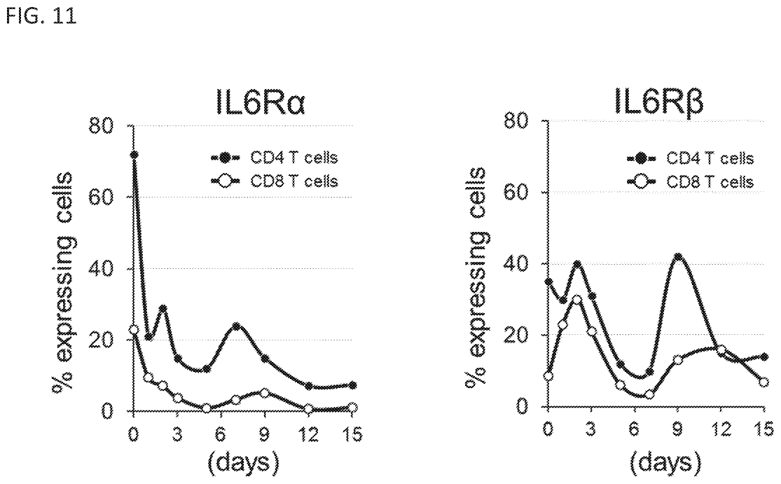

IL6Rα and/or IL6Rβ expressing cells were enriched in less differentiated T cell subsets in both CD4 and CD8 T cells. As shown in FIGS. 7A and 7B, naïve CD4 and CD8 T cells expressed higher levels of IL6Rα and IL6Rβ than the corresponding memory T cells. T cells that expressed both IL6Rα and IL6Rβ were predominantly CD45RA+CD45RO−CD27+CD28+ cells (FIGS. 8A and 8B). Upon TCR stimulation, IL6Rα but not IL6Rβ expression was down-regulated (FIG. 11).

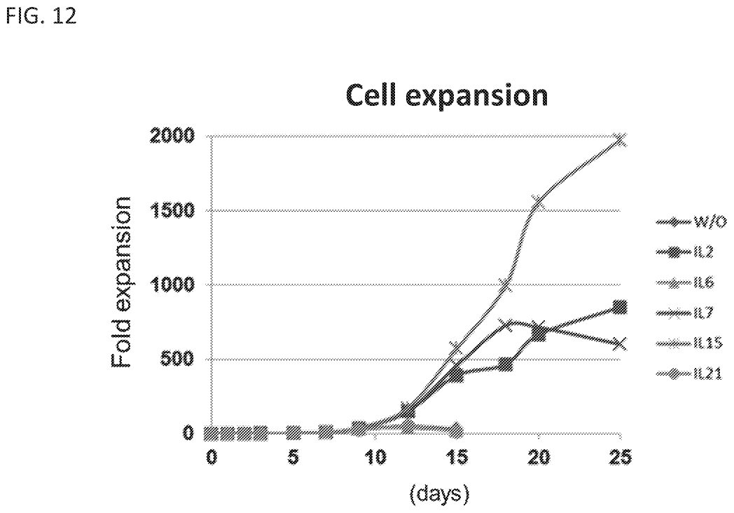

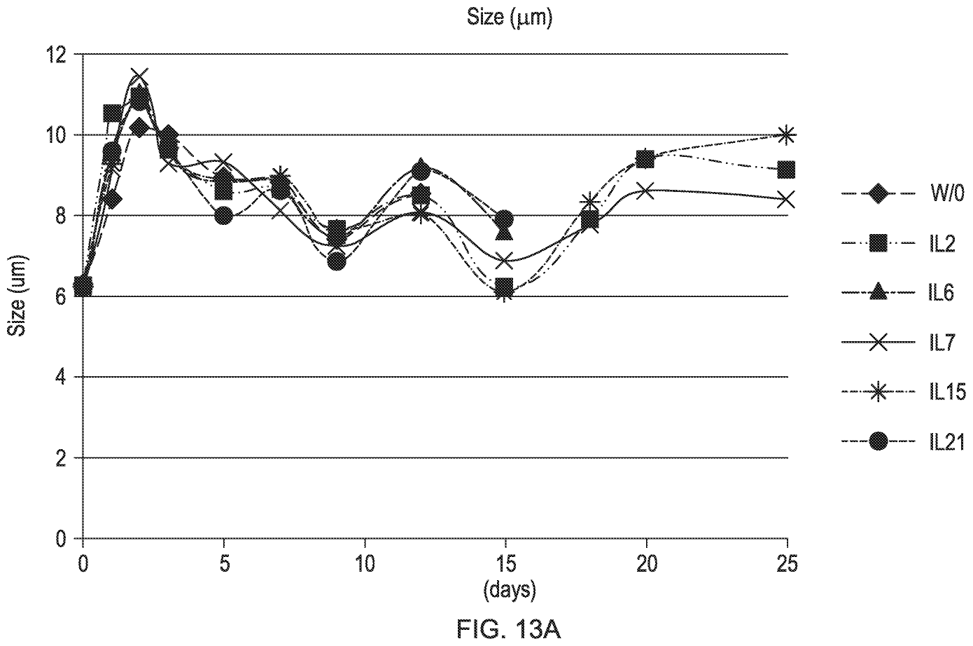

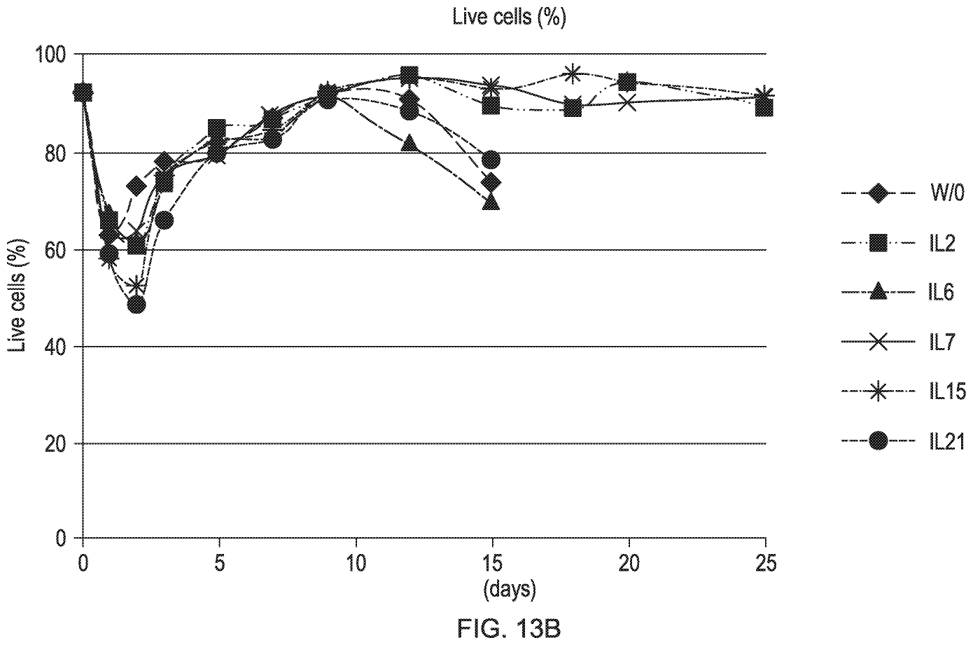

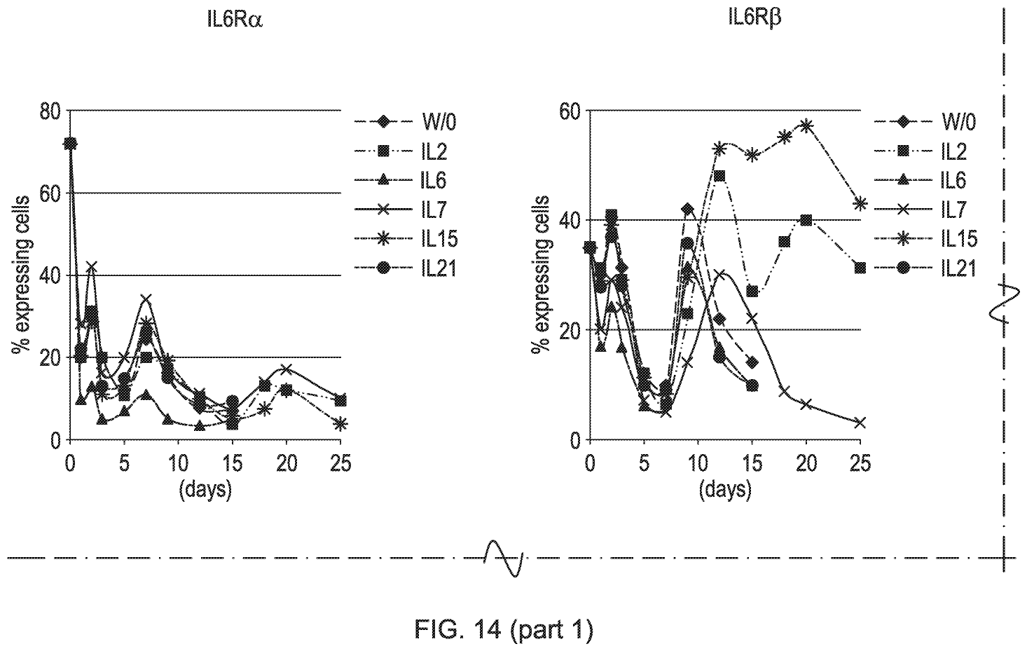

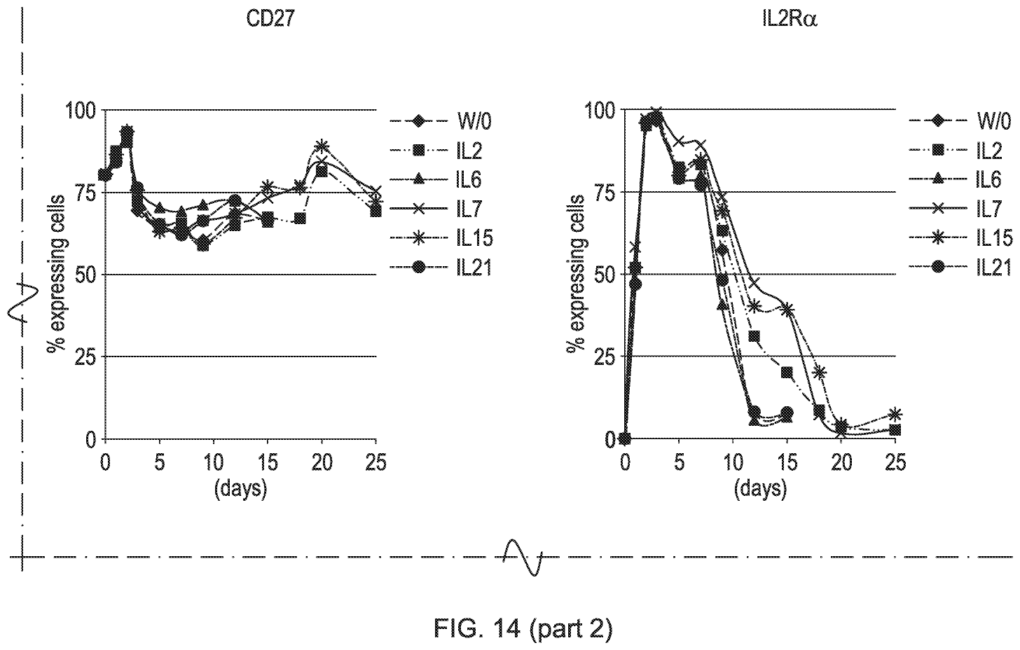

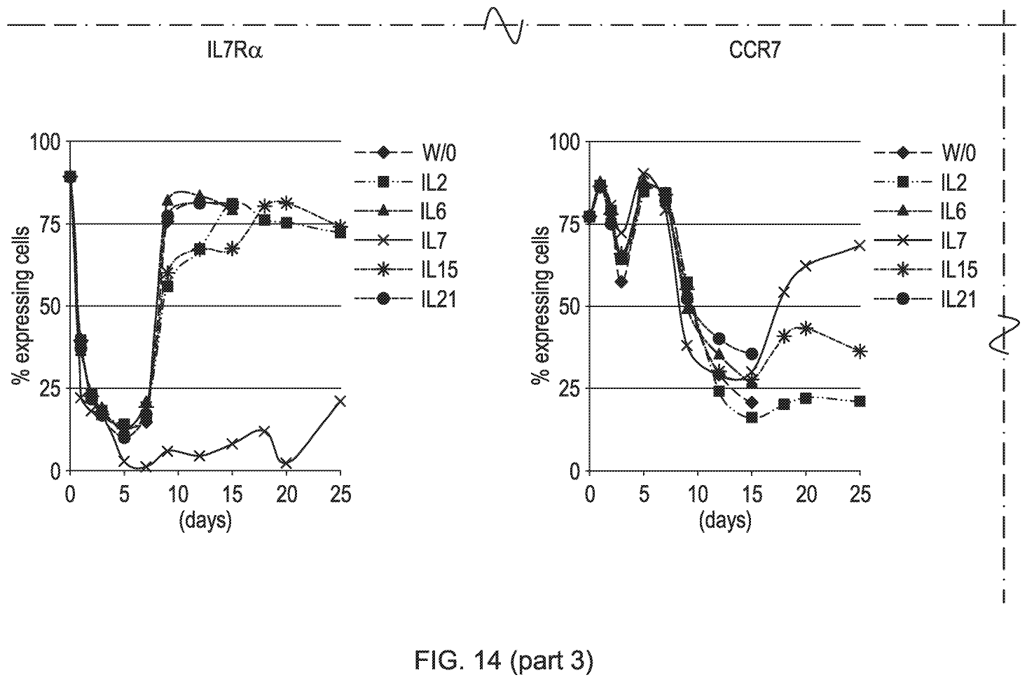

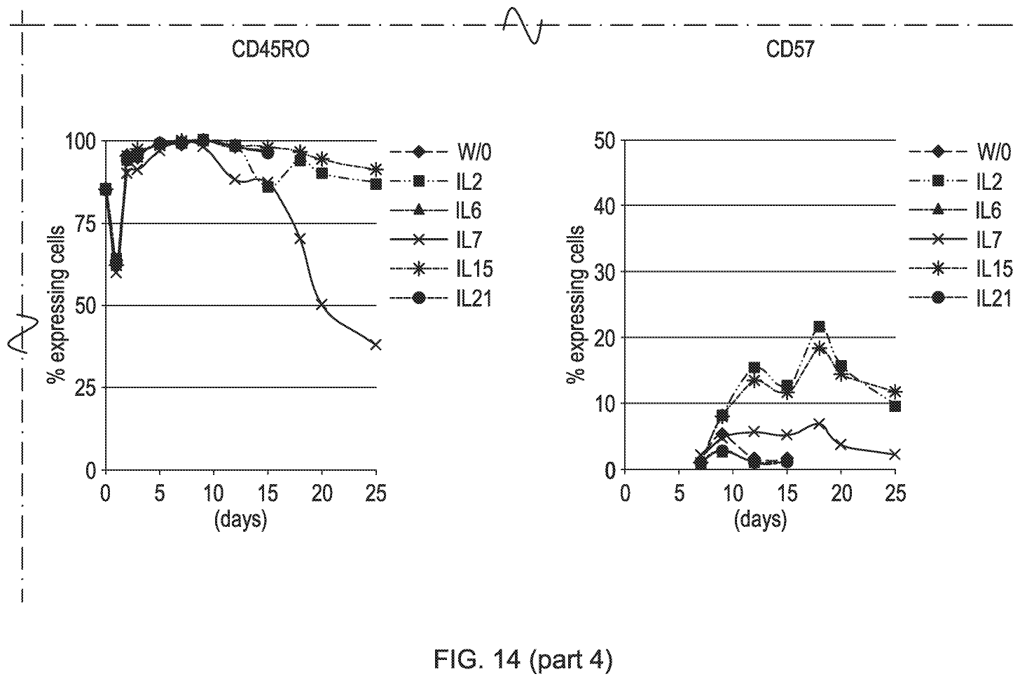

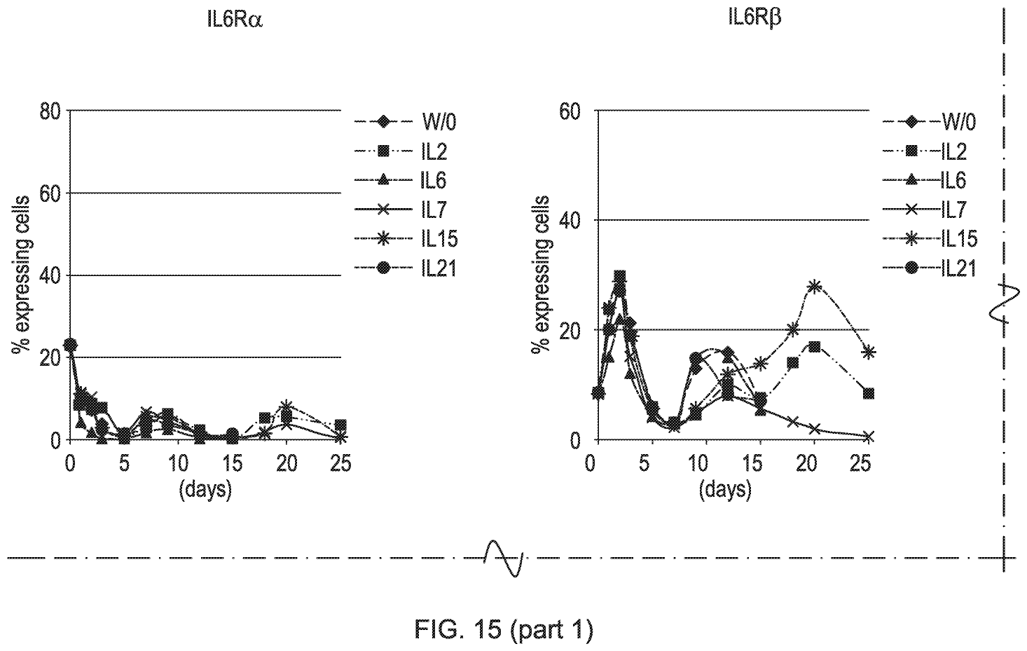

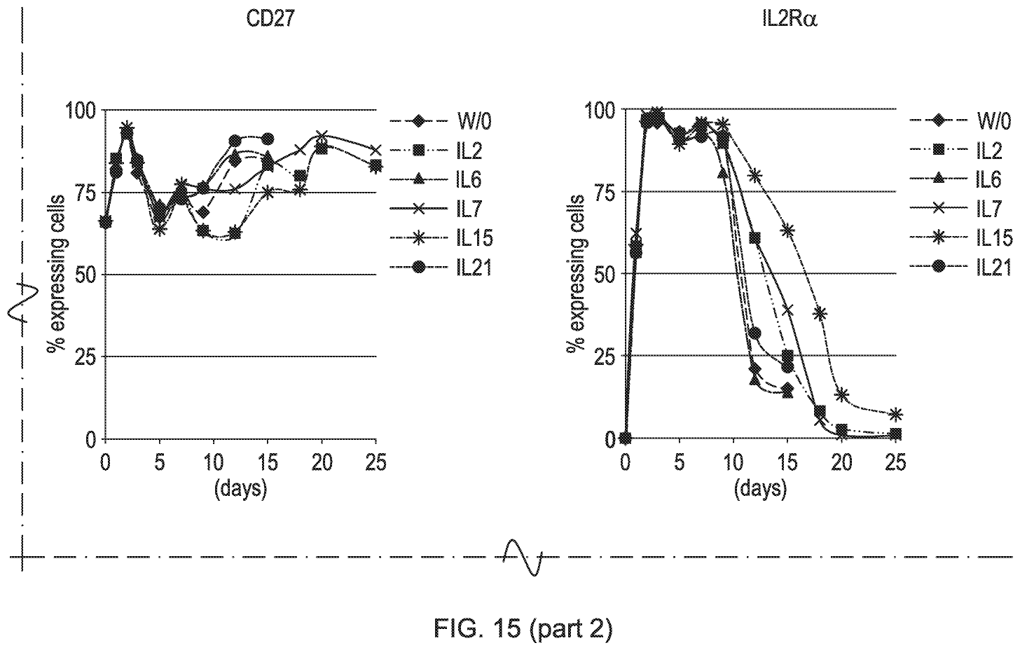

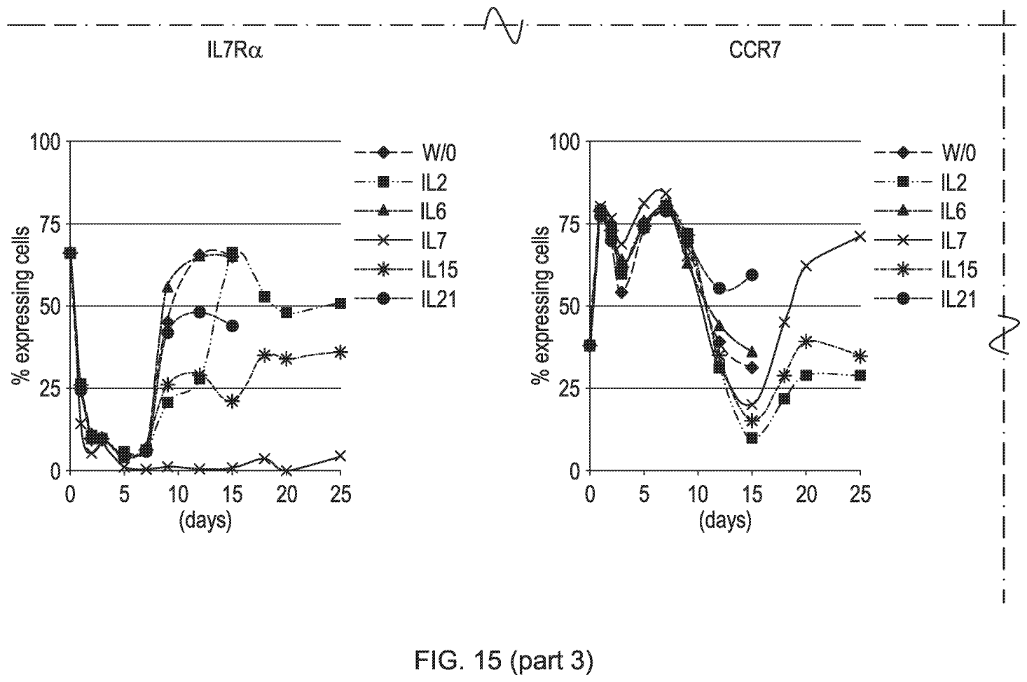

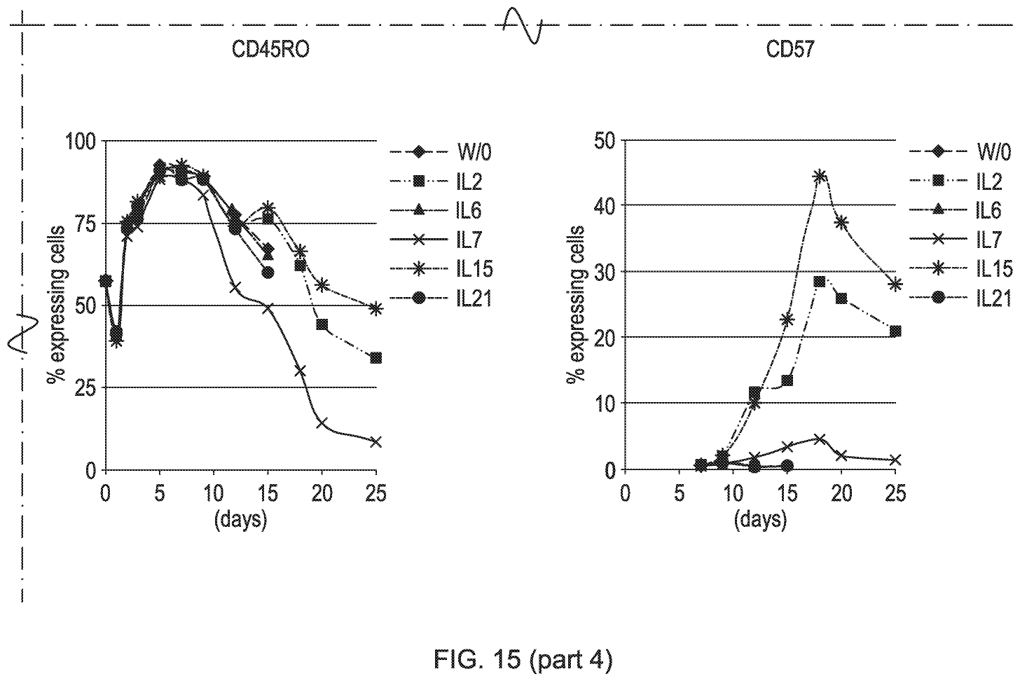

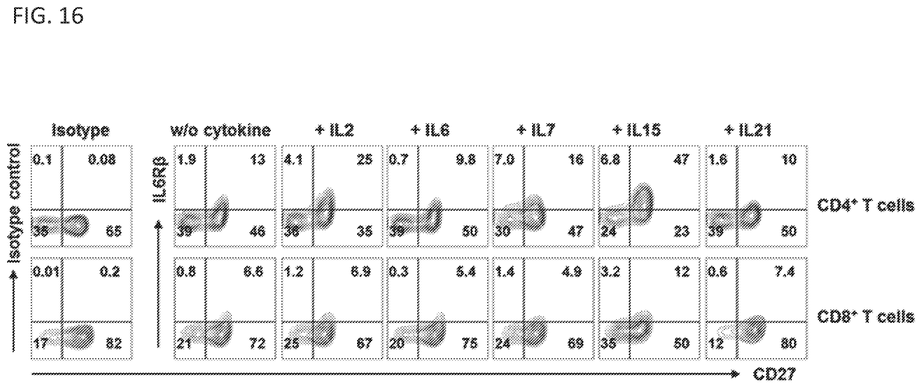

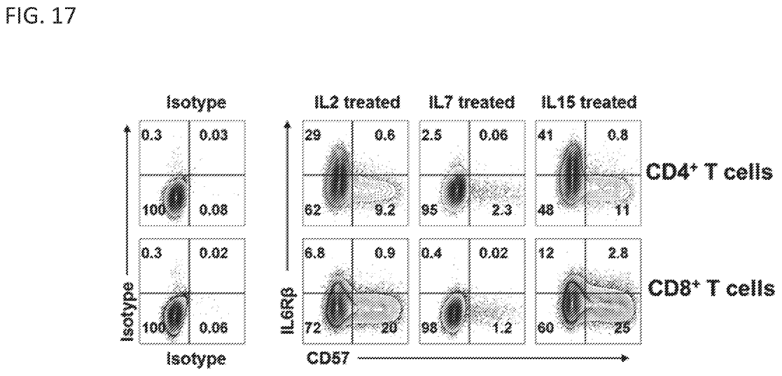

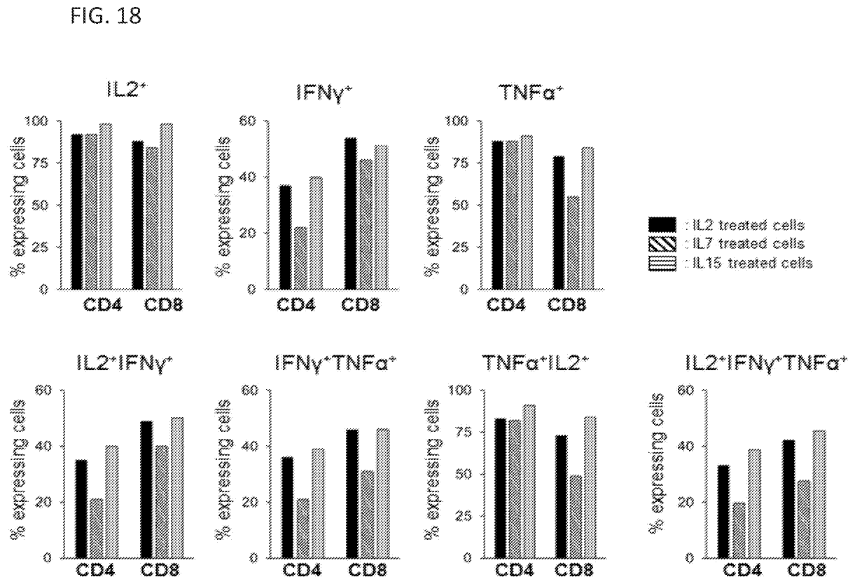

Next, different cytokines were compared for their impact on T cell expansion. Among the cytokines tested, IL15, IL2, and IL7 enhanced T cell expansion, with IL15 showing the greatest enhancement (FIG. 12). Cytokine treatment did not affect cell size (FIG. 13A) or viability (FIG. 13B). IL15 treatment also enhanced expansion of IL6Rβ expressing cells (FIG. 14). IL6Rβ expressing cells were mainly in the CD27+ (FIG. 16) or CD57− (FIG. 17) T cell subsets in both CD4 and CD8 at day 15 after TCR engagement and produced IL2, IFNγ, and TNFα cytokines at day 25 after TCR activation (FIG. 18).

### Example 4: Generation of CARTs with TCR Stimulation for Preclinical Studies

Day 0 unit operations of the engineering runs for preclinical studies began with the manufacturing of the media used on Day 0: Rapid Buffer and Rapid Media (Table 21). The Rapid Buffer (RB) contains the CliniMACS® buffer (Miltenyi) with 0.5% HSA. The Rapid Media (Table 21) was formulated on Day 0 of manufacturing and the base media contains the off-the-shelf media called OpTmizer™ which has Glutamax, IL-2, CTS™ supplement, and ICSR. The Prodigy® machine was primed for use on Day 0.

TABLE 21
Media type and point of use during CART manufacturing

| Media/Buffer Type | Composition | Point of Use |
|---|---|---|
| Rapid Buffer (RB) | CliniMACS ® Buffer | Day O Processing on Cell |
| | (+0.5% human serum | Wash/Separator |
| | albumin (HSA)) | |
| Rapid Media (RM) | OpTmizer ™ Media, | Day 0 for Processing on |
| | CTS ™, IL-2, | Cell Wash/Separator and |
| | Glutamax and ICSR | Cell Seeding |
| Harvest Buffer (HB) | PBS no EDTA and | Harvest Wash Buffer |
| (also called Harvest | 2% HSA | (Day 1) |
| Buffer Solution) | | |
| Cryomedia | Cryostor10 (CS10) | Harvest Formulation |

As the Prodigy® machine was priming on Day 0, the healthy donor leukapheresis material was thawed and the apheresis material was combined into a 600-mL transfer bag that can later be welded onto the Prodigy®. An IPC sample was extracted from the 600 mL transfer bag and measured by NC200 to obtain both the viable cell count and the viability percentage for the starting apheresis material. After priming of the Prodigy® was finished, the apheresis material was transferred to the application bag. After the apheresis entered the Prodigy® machine after initiation of the TCT program, the program ran from 3 h 45 min to 4 h 15 min depending on how many positive selection separations it performed. The TCT program on Day 0 washed out the DMSO in the Centricult with the Rapid Buffer, performed a platelet wash, volume reduction, incubation of the apheresis with the CD4 and CD8 Microbeads in the Centricult, and then selection of the T cells with the Microbeads via positive selection using the magnet on the Prodigy®. The T cells selected with the CD4 and CD8 reagents were eluted into the reapplication bag with the Rapid Media. An in-process control (IPC) sample was taken from the reapplication bag to determine the total viable cell number available for seeding in the culture vessel (G-Rex500MCS).

The G-Rex culture device was first primed with the Rapid Media and then the target cell volume from the reapplication bag was added to the culture vessel. The activation reagent (TransACT) was then added to the culture vessel. The lentiviral vector was then added to the culture vessel after the introduction of TransACT and the vector addition was performed using a MOI of 1.0. The G-Rex500MCS culture vessel was then flushed with the Rapid Media to a final media volume of 250 mL plus the volume of the vector addition. The G-Rex culture vessel was then placed into the incubator to allow the culture to incubate for a target 24 h with a range of 20-28 hours.

After the target 24 h incubation, the CART culture was taken out of the incubator and a sample was extracted to obtain the viable cell count and viability of the cell culture before the Harvest Wash. The sample take at Pre-Harvest was an IPC and was used as an input into the LOVO wash device to determine the flow rate of cells into the spinning filtration membrane. The LOVO used the viable WBC concentration as the IPC. The program used for the CART manufacturing process was described as 4 Washes with one solution and utilized the Harvest Buffer (PBS+2.0% HSA). During the LOVO wash, the IPC bag was used to both reduce the volume and wash the cells with Harvest Buffer before it was finally eluted into the output bag. The output bag from the LOVO wash was then sampled to obtain the viable cell count and viability in order to perform the manual centrifugation with the sanisure bottle and to perform the final steps of the final formulation with the cryomedia.

### Example 5: Generation of BCMA CARTs Using the Activated Rapid Manufacturing (ARM) Process

### Summary

This example describes a CART manufacturing process called “activated rapid manufacturing (ARM).” In some embodiments, cells (for example, T cells) are cultured in a cell culture device containing media (for example, serum-free media, for example, OpTmizer™ media), recombinant human IL-2 (for example, OpTmizer™ media containing OpTmizer™ supplement, GlutaMAX and 100 IU/ml of IL-2), anti-CD3/anti-CD28 (for example, TransAct) and a vector (for example, a lentiviral vector) encoding a BCMA CAR. After 24 hours, the cells, referred as “day 1 CART product” are harvested, sampled, and formulated. Without wishing to be bound by theory, brief CD3 and CD28 activation, for example, using anti-CD3/anti-CD28 (for example, TransAct), promotes efficient transduction of self-renewing T cells. In some cases, some cells are harvested at 48 h, 72 h, and 96 h or 7 days after culture for measuring BCMA CAR expression kinetics in vitro. The day 1 CART responses include, but are not limited to, in vivo cytolytic activity and expansion.

### Generation of Day 1 BCMA CARTs Using the ARM Process

In some embodiments, the activation process provided herein starts with a frozen or fresh leukapheresis product. After a sample for counting and QC is obtained, the product is attached to a cell sorting machine (for example, an installed CliniMACS® Prodigy® device kit) and the program begins. The cells are washed and incubated with microbeads that bind to desired surface markers, such as CD4 and CD8. The bead-labeled cells are selected by passing the cells through a magnetic column. Isolated cells are washed again and the separation buffer is exchanged for cell media. Purified T cells then either proceed to culture or are cryopreserved for later use. Purity of the isolated T cells will pass a QC step by flow cytometry assessment. Cryopreserved cells can be thawed, washed in pre-warmed cell media, and resuspended in cell media. Fresh cells can be added to culture directly. The cells are seeded into membrane bioreactors at 0.4-1.2e6 cells/cm² of membrane, an activating reagent, such as anti-CD3/anti-CD28 beads/polymers, nanoparticles, or nanocolloids, is added, and cell media is added to a final volume of 0.25-2 ml/cm² of membrane. At the time of plating, the cells are transduced with a lentiviral vector encoding BCMA CAR at various multiplicity of infections (MOIs). The titer and the MOI are measured based on cell lines such as SupT1. At 24 hours, the cells are washed to remove unnecessary reagents before staining to measure the CAR expression by flow cytometry and reformulated in cryopreservation media as “day 1 CART product” for in vivo study.

Described in this example are the generation and characterization of T cells expressing BCMA CAR R1B6, R1F2, R1G5, PI61, B61-02, B61-10, or Hy03, manufactured using the ARM process. The sequences of R1B6, R1F2, and R1G5 are disclosed in Tables 3-6. The sequences of PI61, B61-02, and B61-10 are disclosed in Tables 7-11. The sequences of Hy03 are disclosed in Tables 12-15.

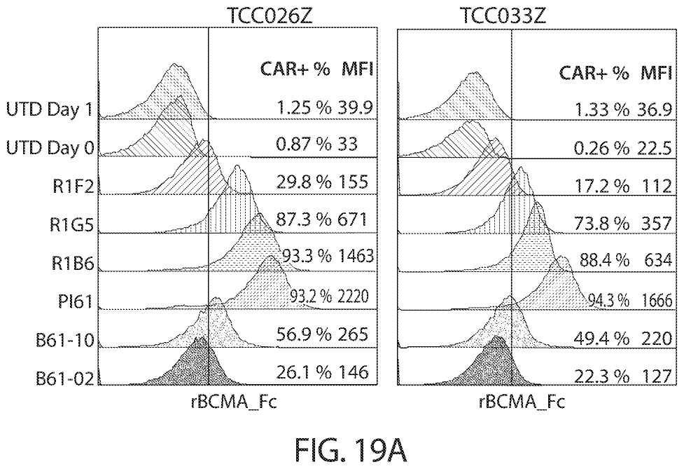

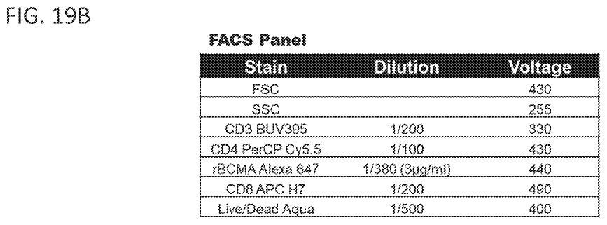

Twenty-four hours after T cells were transduced using lentiviral vectors encoding BCMA CARs at a MOI of 2.5, the expression of CAR was measured by flow cytometry using rBCMA_Fc. As shown in FIG. 19A, it was observed that the whole population of the live CD3+ T cells shifted to the right at different degrees. Cells transduced to express R1G5, R1B6 or PI61 showed the highest CAR expression (FIG. 19A). This pattern of expression as measured by flow cytometry was different from a typical flow cytometry histogram of cells transduced to express a CAR, where a CAR positive population is clearly separated from a negative population. FIG. 19A indicates that there may be “pseudotransduction or transient expression” detected by rBCMA_Fc, which does not always indicate real gene expression. It has been previously reported that lentiviral pseudotransduction was observed beginning at the time of vector addition and lasting up to 24 hours in CD34+ cells and up to 72 hours in 293 cells (Haas D L, et al. Mol Ther. 2000. 291:71-80). Integrase-defective lentiviral vector caused transient eGFP expression for up to 10 days in CD34+ cells and for up to 14 days in 293 cells. Though lentiviral pseudotransduction has not been extensively studied in T cells, this possibility of transient expression in such a short time cannot be ruled out. Therefore, in vitro kinetic study was performed to measure CAR expression of cells manufactured using ARM as indicated below.

### In Vitro CAR Expression Kinetics Study of Cells Manufactured Using the ARM Process

The study described here examines how cells manufactured using the ARM process express CAR molecules over time. Briefly, T cells from a healthy donor were manufactured to express a BCMA CAR using the ARM process at a MOI of 1 and were kept in culture for different time periods and harvested at 24 h, 48 h, 72 h, 96 h, and day 7 for assessing CAR expression kinetics by flow cytometry using AF647 labeled rBCMA_Fc. Understanding the CAR expression kinetics helps to find a surrogate time point for real and stable expression for in vivo triage or clinical dosing strategy.

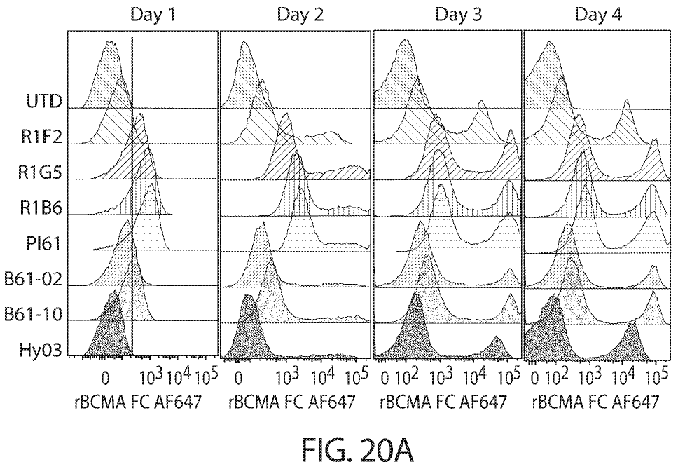

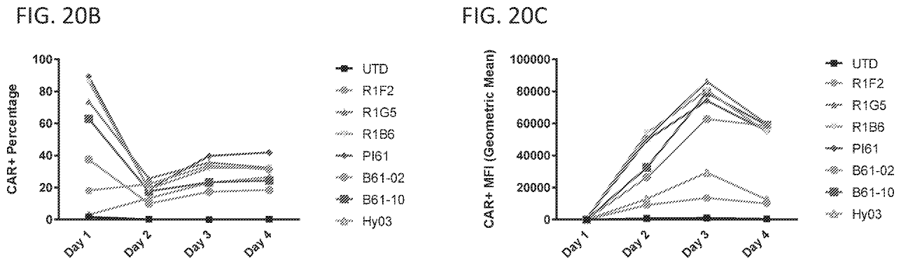

At day 1, the CAR expression pattern of cells transduced at a MOI of 1 (FIG. 20A) is similar to that of cells transduced at a MOI of 2.5 (FIG. 19A). Both MOI conditions showed a pseudo or transient expression pattern at day 1 (FIGS. 19A and 20A). However, at day 2, a rBCMA_Fc positive population started to be separated from the UTD negative control group (FIG. 20A). At day 3 and day 4, a rBCMA_Fc positive population, which represents the BCMA CAR-expressing cells and is absent in the UTD group, clearly showed up in all the groups where cells were transduced to express a BCMA CAR. From day 3 to day 4, the CAR+% was relatively stable for each CAR construct (FIG. 20B), with the highest MFI observed at day 3 (FIG. 20C) (the cells were the largest at this time point). Consistent with the data shown in FIG. 19A, cells transduced to express PI61, R1G5 and R1B6 were the highest CAR expressers (FIG. 20A). Notably, cells transduced with vectors encoding R1F2 or Hy03 did not show transient CAR expression at day 1 but clearly expressed BCMA CAR molecules later at day 3 and day 4 (FIG. 20A). In conclusion, vectors encoding different CARs may have different CAR expression kinetics over time, and day 3 was chosen as a surrogate time point for CAR expression.

### Evaluating Functionality of the Day 1 ARM Processed BCMA CART In Vivo

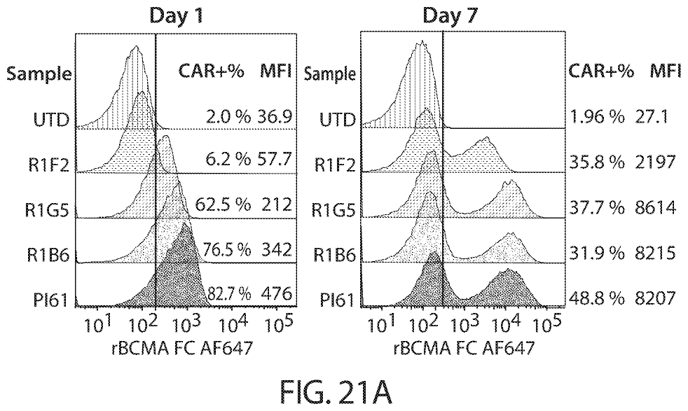

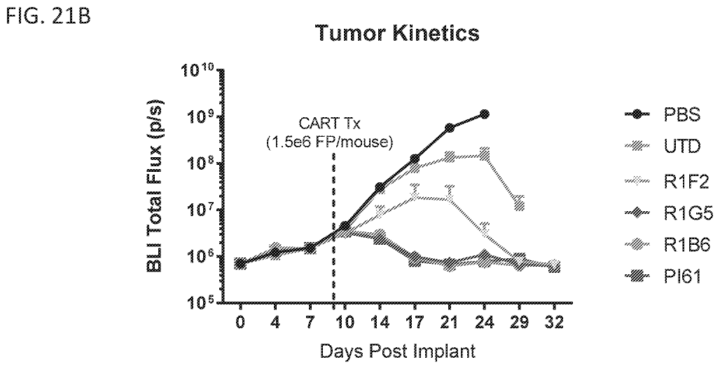

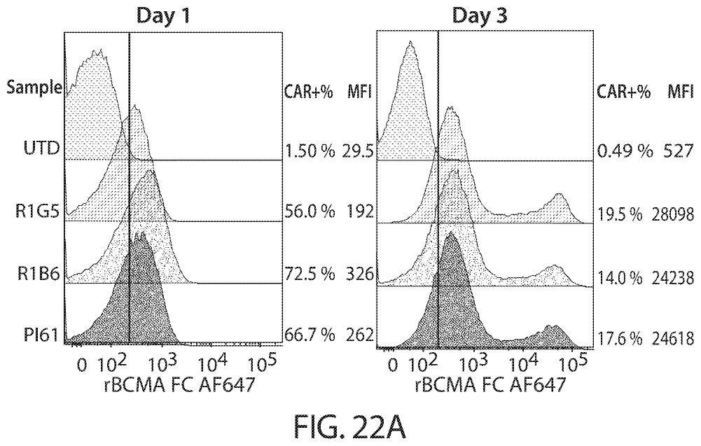

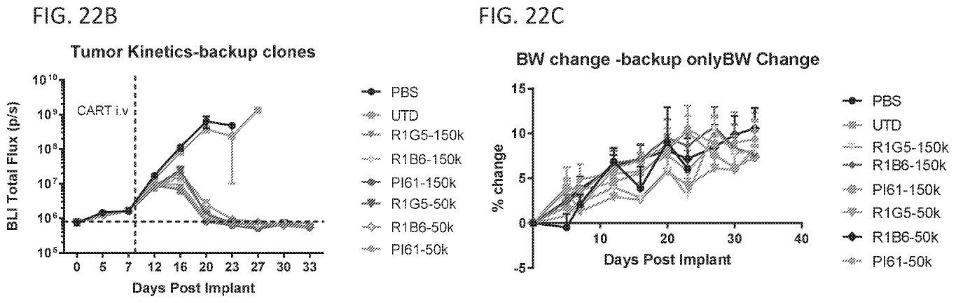

The day 1 CARTs were examined for their anti-tumor activity in vivo using a disseminated KMS-11-luc multiple myeloma xenograft mouse model. The luciferase reporter allows for monitoring of disease burden by quantitative bioluminescence imaging (BLI). Briefly, day 1 CARTs manufactured as described above were administered in tumor-bearing mice. In the first in vivo study (FIGS. 21A and 21B), each mouse received a final CART product at a dose of 1.5E6 cells. CAR expression was analyzed at day 1 and day 7 (FIG. 21A). In the in vivo efficacy study, cells expressing PI61, R1G5 or R1B6 demonstrated potent anti-tumor activities (FIG. 21B). Cells expressing RIF2 showed a delayed efficacy (FIG. 21B). The UTD group also showed partial anti-tumor activity 14 days after CART injection, which could be due to alloreaction (FIG. 21B). A second in vivo study tested dose titration of the CAR+ T cells. The doses of CAR+ T cells were based on CAR+% at day 3 (FIG. 22A). Tumor intake kinetics was monitored twice a week by BLI measurement. FIG. 22A shows CAR expression detected at day 1 and day 3. The in vivo results indicate that all three clones PI61, R1B6 and R1G5 at both doses of 1.5e5 CAR+ T cells and 5e4 CAR+ T cells were able to reject and clear tumor as shown in FIG. 22B. FIG. 22C shows body weight changes over the course of this study, displaying no indication of GVHD.

### Example 6: Kinetics of Rapid CARTs Harvested Between 12-24 Hours

### Introduction

To determine whether a rapid CART product could be generated in less than 24 hours, the kinetics for harvesting rapid CARTs generated after 12-24 hours in culture was characterized. This evaluation was performed at small scale using T cells enriched from cryopreserved healthy donor apheresis and simultaneous addition of TransAct activation reagent and technical grade CTL019 vector at seeding. Primary readouts were viability, viable cell recovery post-expansion, leukocyte and T cell subset composition, and transduction efficiency (as determined via surface immunophenotyping) on freshly harvested CART products.

### Methods

Lentivirus production and titer determination: The lentiviral vector encoding CTL019 was prepared with a HEK293T-based qPCR titer of 4.7×10⁷ TU/mL and an approximated T cell-based titer of 1.88×10⁷ TU/mL.

T cell isolation: A cryopreserved leukopak (LKPK) of healthy donor apheresis was obtained from Hemacare and stored in liquid nitrogen until needed. On Day 0, the apheresis was thawed until a small ice crystal remained, and then diluted with Prodigy® process buffer. Automated CD4/CD8 positive selection was then performed on the CliniMACS® Prodigy® with the TS 520 tubing set and T Cell Transduction (TCT) program software version 1.0. The final Prodigy® product was eluted in OpTmizer™ complete T cell medium, and cell concentration and viability were determined by AO/PI staining as enumerated by the Cellometer Vision (Nexcelom).

Culture initiation and transduction: Cells from the Prodigy® product were immediately seeded into a total of seven vessels: five vessels for transduced cultures and two vessels for untransduced (UTD) cultures. At timepoint zero, each vessel was seeded at a density of 0.6×10⁶ viable cells per cm² of membrane, plus GMP-grade TransAct, and brought to a final concentration of 1.2×10⁶ viable cells/mL with OpTmizer™ complete T cell media containing IL-2. Vector was thawed at room temperature and added to each transduced culture at a MOI of 0.45 based on the approximated T cell titer. No virus was added to the UTD controls. Once seeded, cultures were incubated at 37° C. and 5% CO₂ until ready for harvest.

Harvest: At each timepoint 12 to 24 hours after culture initiation, one transduced culture was selected for harvest. Cells were harvested by swirling the vessel to gently resuspend the cells off the membrane, then the full culture volume resuspended and transferred by serological pipette to a conical tube. A small aliquot was taken for a pre-wash count, viability determination, and flow staining. The remainder of each culture was washed twice in 50 mL (twice in 100 mL for UTD vessels), resuspended, and a post-wash aliquot taken to examine counts and viability.

Flow cytometry of leukocyte composition and CD19-CAR expression during CART manufacturing: In-process samples before and after culturing were stained for leukocyte composition, T cell phenotype, and CAR expression where applicable. CTL019-CAR expression on transduced T cells was evaluated using a custom-ordered fluorophore-labeled anti-idiotype antibody (eBioscience). At each harvest timepoint, aliquots of the culture were immediately stained with viability dye (Biolegend), washed, then stained with two flow panels both containing a CD3 stain and the anti-idiotype antibody and fixed in paraformaldehyde for acquisition. Samples were measured on a flow cytometer (BD LSRFortessa; single color controls were used for compensation), and data was analyzed with FlowJo software. For analysis, all samples stained for leukocyte composition were pre-gated on viable CD45+ singlet events and all samples stained for T cell subsets were pre-gated on viable CD3+ singlet events. Gates for CD45RO and CCR7 were established using fluorescence minus one (FMO) controls.

### Results

The leukocyte composition of the LKPK, Prodigy® product before culture, and the CART products after culture were characterized using flow cytometry on Day 0 and each harvest time point. The cell types identified were T cells (CD3+), monocytes (CD14+), B cells (CD19+), natural killer (NK) cells (CD3-56+), and other cells (Table 22). Prodigy® enrichment produced a Day 0 starting material that was highly viable (92.9%) and enriched for T cells (from 48% to 92%) while reducing contaminating B cells (6% to 0.10%) and monocytes and NK cells to under 4% each. After 12-24 hours in culture, the purity of the viable cells increased an additional 3-4.4%, corresponding with an immediate reduction of monocytes and B cells by hour 12 and gradual reduction of NK cells between hours 12 and 24. Of the leukocytes that express extracellular CAR by flow cytometry, less than 3% were contaminant cells (i.e. not T cells), with the greatest jump in CAR purity (96.6% to 99.2%) occurring between 15 and 18 hours after seeding.

TABLE 22
Gross leukocyte composition of CART products

| | | % of population | | | | |
|---|---|---|---|---|---|---|
| | Product or | | | | CD3− | |
| Timepoint | Subpopulation | CD3+ | CD14+ | CD19+ | CD56+ | Other |
| Day 0 | LKPK | 48% | 29% | 6.0% | 11.6% | 5.0% |
| | Prodigy ® Product | 92% | 3% | 0.10% | 3.7% | 0.4% |
| CARTs | 12 hr | 95.3% | 0.2% | 0.02% | 3.3% | 1.1% |
| pre-freeze | 15 hr | 95.6% | 0.2% | 0.01% | 3.3% | 0.9% |
| | 18 hr | 96.4% | 0.1% | 0.0% | 2.7% | 0.9% |
| | 21 hr | 96.3% | 0.2% | 0.0% | 2.3% | 1.2% |
| | 24 hr | 96.2% | 0.2% | 0.0% | 2.2% | 1.5% |
| | 24 hr UTD (n = 2) | 96.4% | 0.1% | 0.06% | 2.4% | 1.1% |
| | 12 hr (of CAR+ only) | 97.1% | 0.6% | 0.0% | 2.4% | 0.0% |
| | 15 hr (of CAR+ only) | 96.6% | 0.9% | 0.0% | 2.5% | 0.0% |
| | 18 hr (of CAR+ only) | 99.2% | 0.1% | 0.0% | 0.7% | 0.0% |
| | 21 hr (of CAR+ only) | 99.1% | 0.3% | 0.0% | 0.7% | 0.0% |
| | 24 hr (of CAR+ only) | 98.9% | 0.3% | 0.0% | 0.8% | 0.0% |

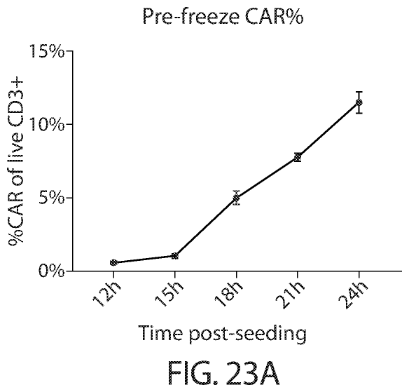

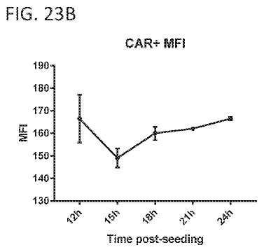

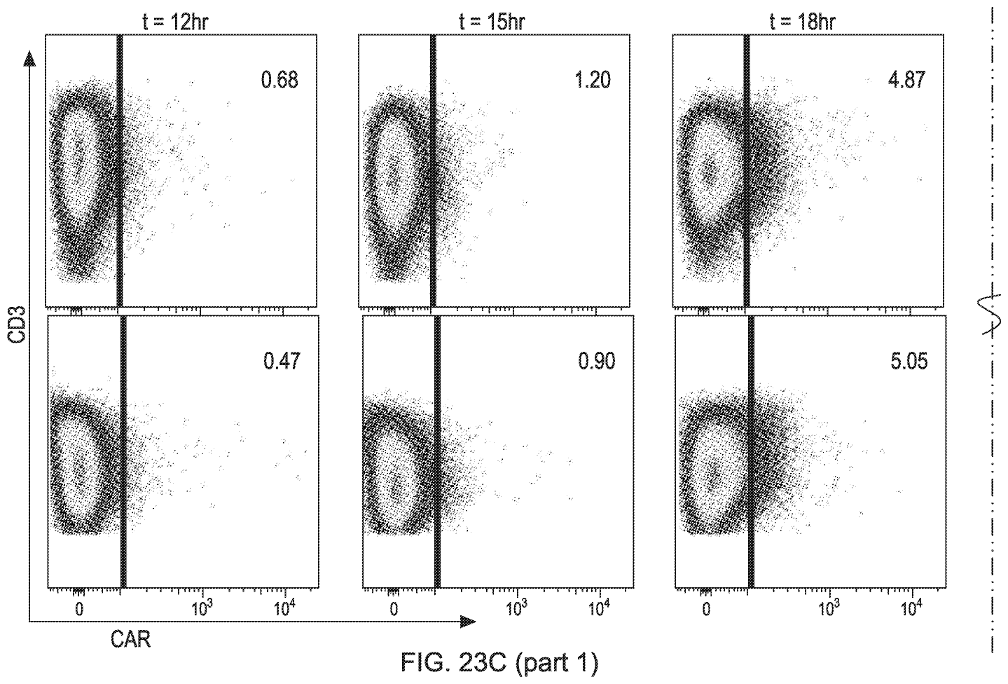

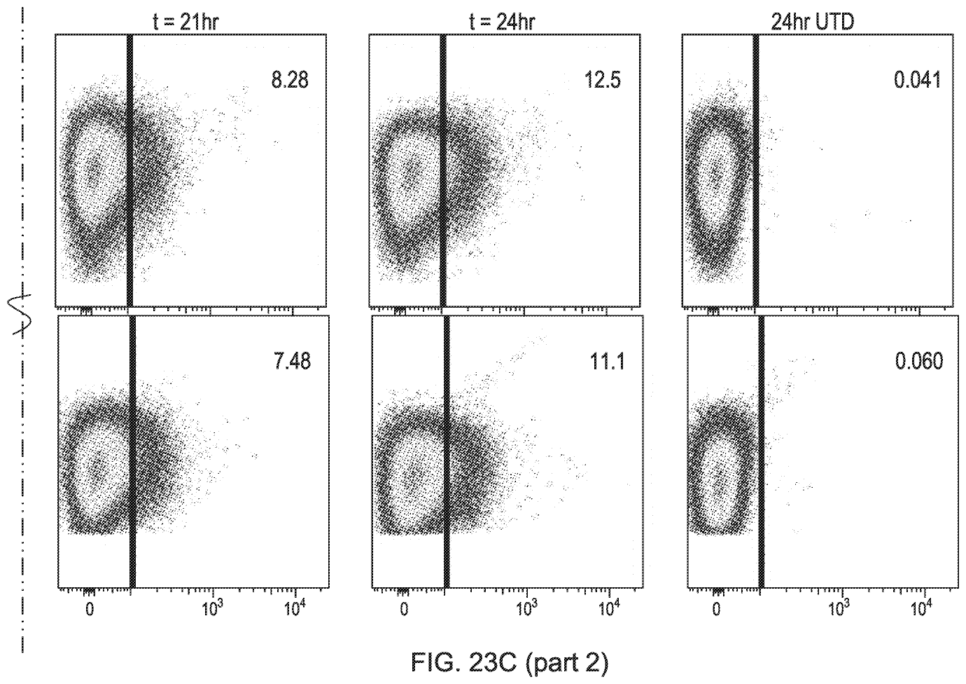

The increase in purity of CAR-expressing cells 18 hours into culture (Table 22) coincides with an increase in the percentage of T cells with CAR surface expression (FIGS. 23A and 23C). As observed previously with rapid CART products evaluated by flow cytometry after 24 hours in culture (see Example 5), CAR surface expression did not lead to distinct positive and negative populations. Gating for CAR positivity was therefore established using the UTD samples as the lower bound. The proportion of CD3+ cells expressing extracellular CAR remained below 1% until 15 hours post-seeding; and CAR expression then increased 3-4% every three hours to a maximum of 11.8% without saturating (FIG. 23A). The intensity of CAR expression as determined by MFI also increased slightly >18 hours in culture but remained dim through hour 24 (FIG. 23B).

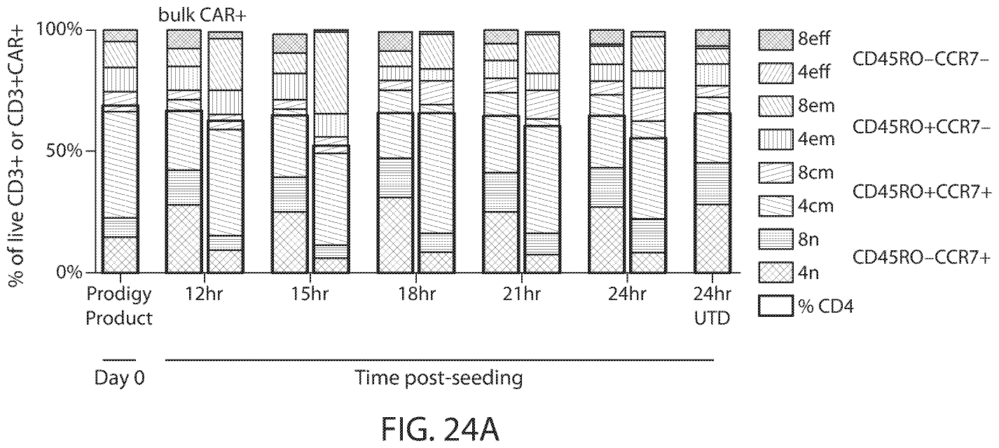

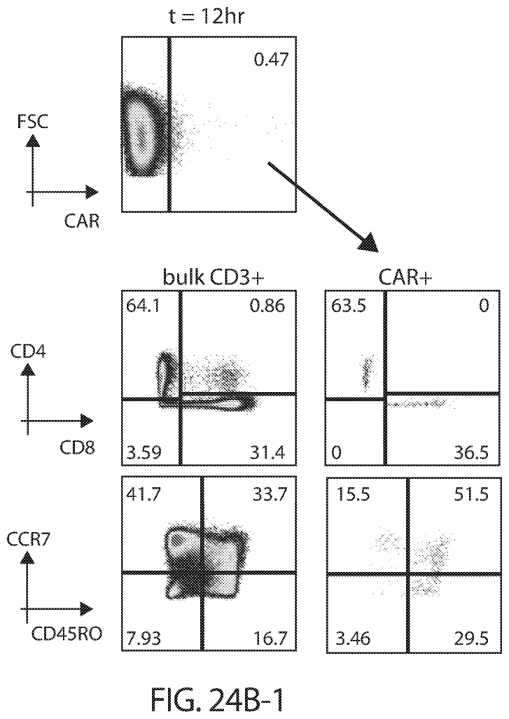

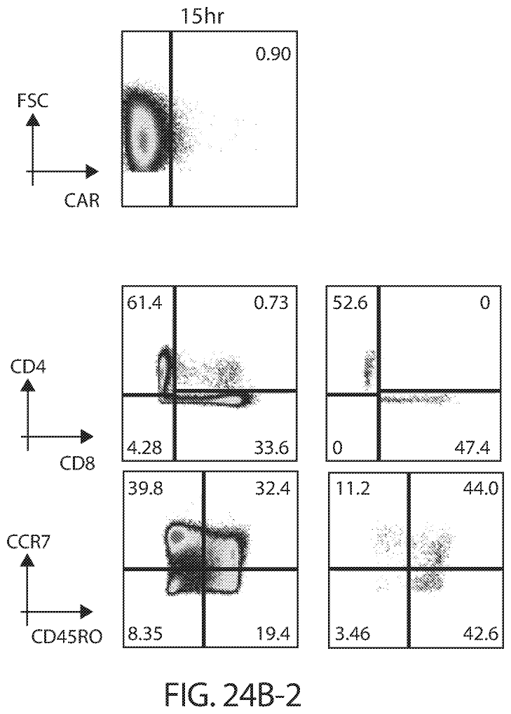

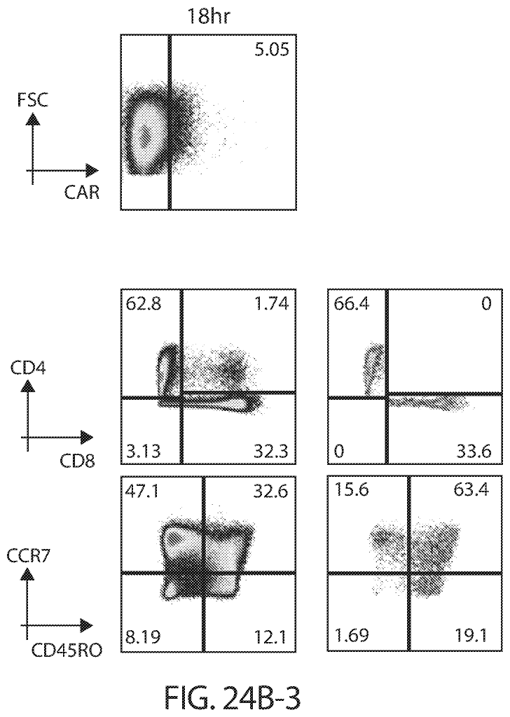

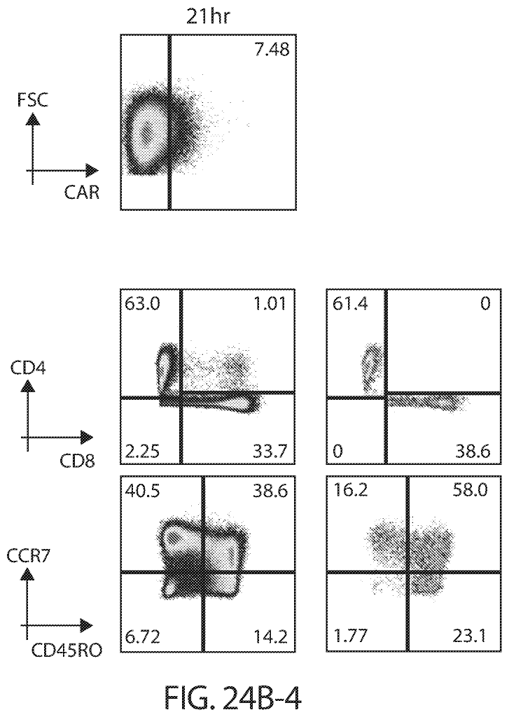

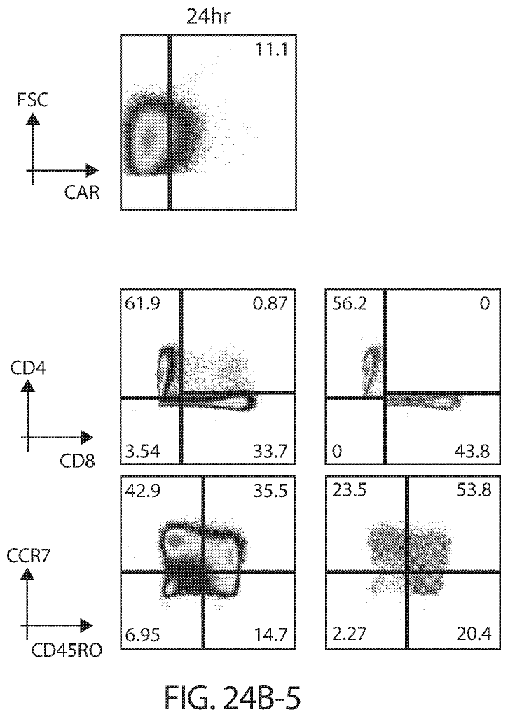

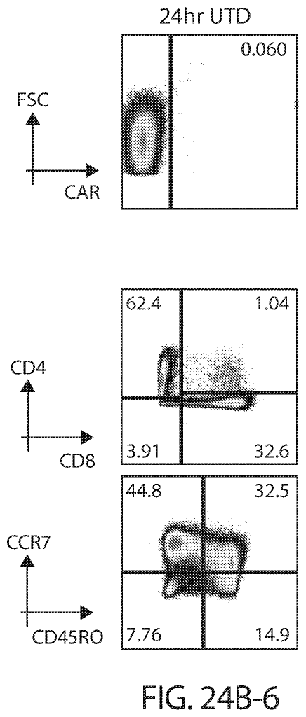

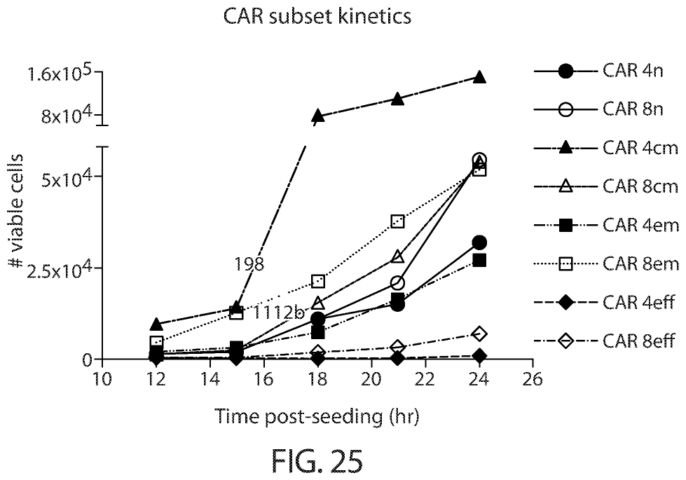

T cell subsets (CD4: CD8 ratio and memory subset composition) were also evaluated at each timepoint (FIGS. 24A and 24B) using a combination of CD4, CD8, CD45RO, and CCR7; where undifferentiated naive-like T cells were defined as CCR7+CD45RO−, central memory cells as CCR7+CD45RO+, effector memory cells as CCR7−CD45RO+, and highly differentiated effector T cells as CCR7−CD45RO−. Across all timepoints evaluated, including the UTD, cultures contained a greater proportion of naive cells (40-47%) and lower proportion of central memory cells (33-39%) than the initial starting material (23% and 52%, respectively). Interestingly, although the frequency of naïve or central memory T cells in the bulk composition did not change between 12 to 24 hours, later harvests were correlated with a greater frequency of extracellular CAR-expressing cells that were naïve and a lower frequency of extracellular CAR-expressing cells that were central memory (16% naïve/63% central memory among CAR-expressing cells at 18 hours vs. 24% naïve/54% central memory among CAR-expressing cells at 24 hours). Similarly, while bulk CD4: CD8 ratio did not change significantly, the CD4 fraction of the CAR+ cells decreased by 10% (66% to 56%) between 18-24 hours. Converting these frequencies to total cell numbers (FIG. 25) reveals that the subsets of T cells that appeared to express the CAR the earliest are mostly naive CD4 cells between 15-18 hours in culture; naive CD8 CARs and central memory CD8 CARs then rapidly increase in frequency.

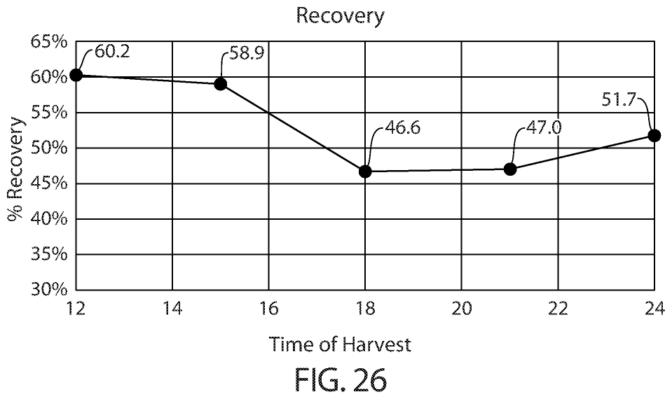

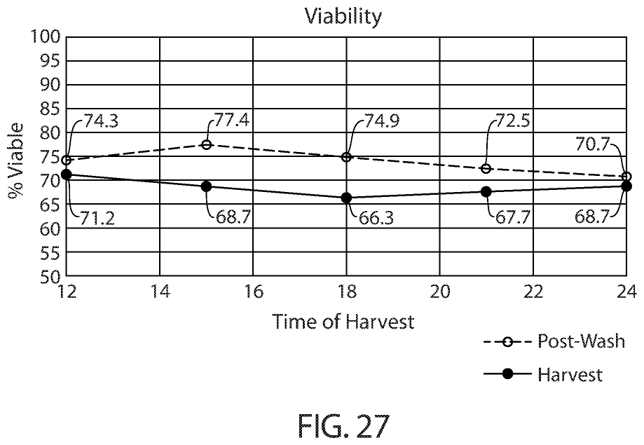

Viable cell recovery (or fold expansion) as well as pre- and post-wash viability were determined at each harvest time point (FIGS. 26 and 27). Recovery of viable cells decreased by 13% until 18 hours post-seeding (lowest 46%, coinciding with the increased rate of extracellular CAR expression), then increased slightly to 52% for cultures harvested at later time points (FIG. 26). Product viability increased after washing to 71-77% with viability decreasing for harvests between 15-24 hours (FIG. 27).

### Conclusion

Of time points tested between 12-24 hours, rapid CARTs seeded simultaneously with TransAct and technical grade CTL019 vector show the highest CAR surface expression at 24 hours. Very few cells are CAR+ (as measured at the time of harvest) until 15 hours post-seeding, after which % CAR increases more rapidly. The intensity of CAR expression is dim but increases slowly after 18 hours post-seeding.

Rapid CART products become purer (greater % T cells) than the starting material at all points between 12 to 24 hours post-seeding due to monocyte loss in the first 12 hours, followed by a minor loss of NK cells and any residual B cells not removed by Prodigy® enrichment.

Although overall cell recovery is lowest when harvested 18 hours post-seeding (improving slightly by 24 hours), the overall T cell composition does not change between 12 and 24 hours post-seeding. T cells that first express extracellular CAR are mostly central memory CD4s between 15 and 18 hours post-seeding, then naive and central memory CD8s show CAR expression.

### Example 7: Description of the Activated Rapid Manufacturing (ARM) Process

In some embodiments, CART cells are manufactured using a continuous Activated Rapid Manufacturing (ARM) process, over approximately 2 days, which will potentially allow for a greater number of less differentiated T cells (T naïve and TSCM (stem central memory T) cells) to be returned to a patient for in vivo cellular expansion. The short manufacturing time period allows the early differentiated T cells profile to proliferate in the body for their desired terminal differentiated state rather that in an ex vivo culture vessel.

In some embodiments, CART cells are manufactured using cryopreserved leukapheresis source material, for example, non-mobilized autologous peripheral blood leukapheresis (LKPK) material. Cryopreserved source material undergoes processing steps for T cell enrichment on the first day of production (Day 0) by means of anti-CD4/anti-CD8 immunomagnetic system. Positive fraction is then seeded in G-rex culture vessel, activated with an anti-CD3/CD28 system (TransACT) and on the same day transduced with a lentiviral vector (LV) encoding a CAR. On the following day, after 20-28 hours of transduction, the T cells are harvested, washed four times, formulated in freezing medium and then frozen by a Controlled Rate Freezer (CRF). From the start of the process on Day 0 to the initiation of harvest on the following day, cells are cultured for 20-28 hours with a target of 24 hours after Day 0 seeding.

Media for Day 0 were prepared according to Table 21. The cryopreserved leukapheresis material is thawed. The thawed cells are diluted with the Rapid Buffer (Table 21) and washed on the CliniMACS® Prodigy® device. The T cells are selected by CliniMACS® CD4 and CD8 microbeads. Once the program is finished for T cell selection (approximately 3 h 40 min to 4 h 40 min), the reapplication bag containing the cells suspended in Rapid Media (Table 21) are transferred in a transfer pack. A sample is taken for viability and cell count. The cell count and viability data from the positive fraction bag is used to determine the cell concentration when seeding the culture vessel for activation and vector transduction.

Following positive selection of T cells via the CliniMACS® microbeads (CD4 and CD8), the cells are seeded in the culture vessel, G-Rex. Once the cells are seeded, the activation reagent (TransACT) is then added to the culture vessel. The cells are then transduced with a lentiviral vector encoding a CAR at a target MOI of 1.0 (0.8-1.2). Following the vector addition, the culture vessel is transported to an incubator where it is incubated for a target of 24 hours (operating range 20-28 hours) at a nominal temperature of 37° C. (operating range 36-38° C.) with nominal 5% CO₂ (operating range 4.5-5.5%). Following the incubation, the cells are washed with Harvest Wash Solution (Table 21) four times to remove any non-integrated vector and residual viral particles, as well as any other process related impurities. Then, the cells are eluted and a sample for cell count and viability is taken for testing and the results are used to determine the volume required to re-suspend the cells for final formulation with CryoStor® CS10. The cells are then centrifugated to remove the Harvest Wash Solution and proceed with cryopreservation.

In some embodiments, the CAR expressed in CART cells binds to CD19. In some embodiments, IL-2 used in the Rapid Media (RM) (Table 21) can be replaced with IL-15, hetIL-15 (IL-15/sIL-15Ra), IL-6, or IL-6/sIL-6Ra.

In some embodiments, the CAR expressed in CART cells binds to BCMA. In some embodiments, IL-2 used in the Rapid Media (RM) (Table 21) can be replaced with IL-15, hetIL-15 (IL-15/sIL-15Ra), IL-6, or IL-6/sIL-6Ra.

### Example 8: Characterization of CD19 CART Cells Manufactured Using the Activated Rapid Manufacturing (ARM) Process

Disclosed herein is an anti-CD19 CAR-T cell product manufactured using the activated rapid manufacturing (ARM) process. The ARM process reduces the turnaround time compared to traditional manufacturing (TM) processes, prospectively allowing a timely infusion of the anti-CD19 CAR-T cell product to patients. Moreover, the ARM process also preserves putative stem memory T (Tₛₜₑₘ) cells, a cellular subset associated with improved antitumor efficacy. The main difference in manufacturing is that while the TM process includes an expansion phase in which anti-CD19 CAR T cells are cultured in vitro for 9 days with interleukin (IL−) 2 before being formulated, the ARM process allows formulation after only 24 hours of culture. This is made possible by the use of a fully biocompatible nanomatrix coupled to monoclonal antibodies (mAb) with agonistic activity against CD3 and CD28, which differently from the CD3/CD28 paramagnetic beads used in the TM process, can be washed away with the residual lentiviral vector right after transduction. Results from a xenograft mouse model, as well as final product enrichment for Tₛₜₑₘ cells, a subpopulation associated with increased persistence and long-term antitumor effects, suggest an overall improved therapeutic potential of anti-CD19 CAR T cells manufactured using the ARM process as compared to anti-CD19 CAR T cells manufactured using the TM process. Another important difference revealed by the xenograft mouse model is a potential delayed cellular kinetics expansion of anti-CD19 CAR T cells manufactured using the ARM process for approximately one week compared to the counterparts manufactured using the TM process. This delay is estimated to be approximately 1 week, which imposes corresponding prolongation of the window for careful monitoring of potential toxicities from 3 weeks, as with anti-CD19 CAR T cells manufactured using the TM process, to 4 weeks. Conversely, non-clinical safety data from an in vitro cytokine release model indicate that anti-CD19 CAR T cells manufactured using the ARM process and those manufactured using the TM process might have a similar potential to induce IL-6 production in vivo and therefore carry a similar cytokine release syndrome (CRS) risk. Based on this evidence, anti-CD19 CAR T cells manufactured using the ARM process will be investigated in a Phase I, open label clinical study in patients with advanced small lymphocytic lymphoma (SLL)/chronic lymphocytic leukemia (CLL) in combination with the Bruton tyrosine kinase inhibitor (BTKi) ibrutinib (Imbruvica), an already approved drug in this indication, and as single agent in DLBCL.

### Generation and In Vitro Analysis

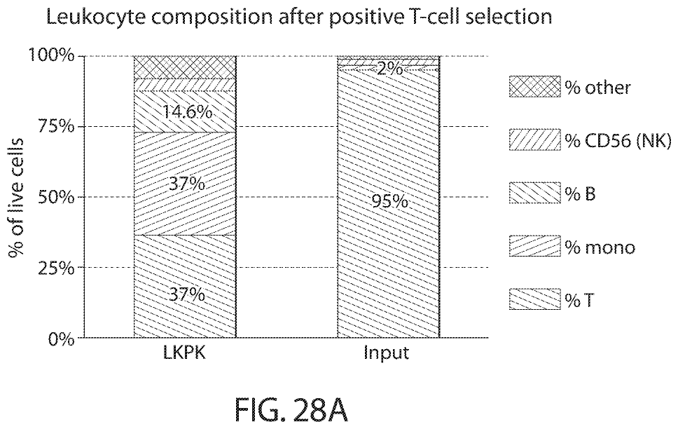

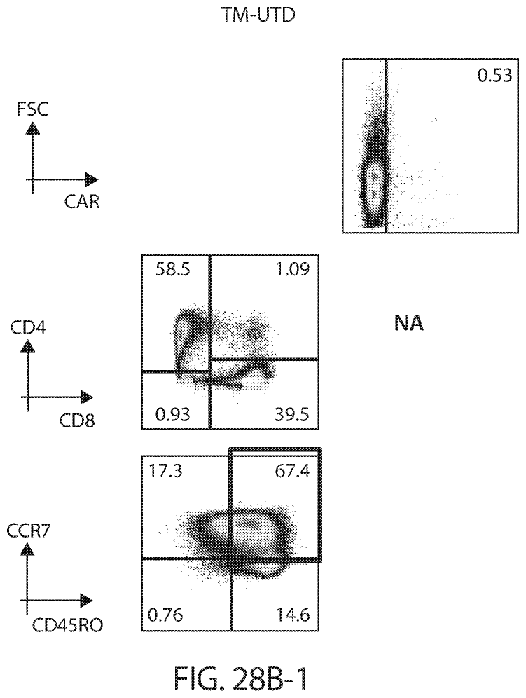

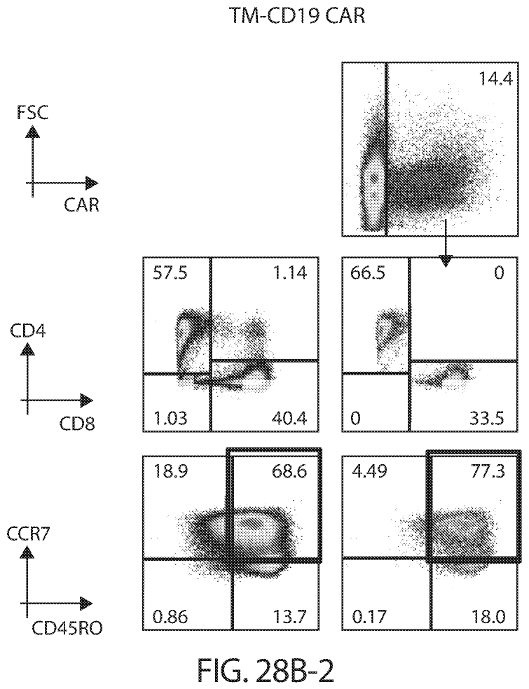

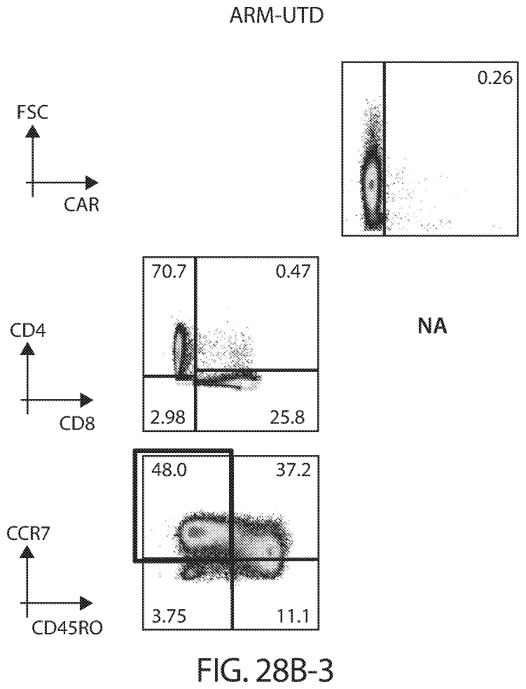

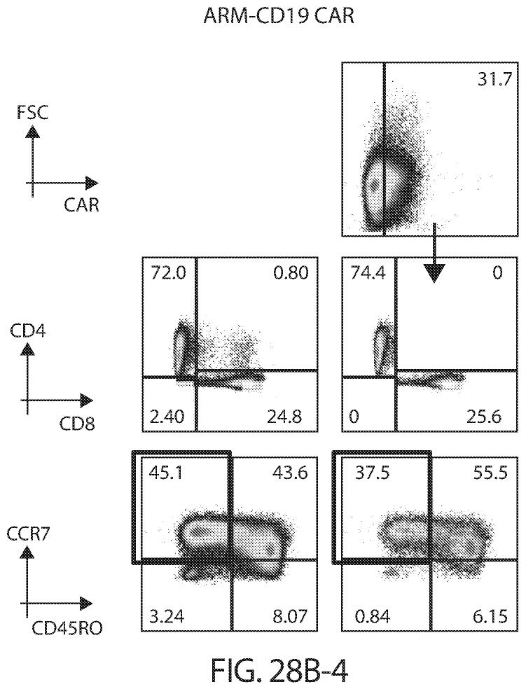

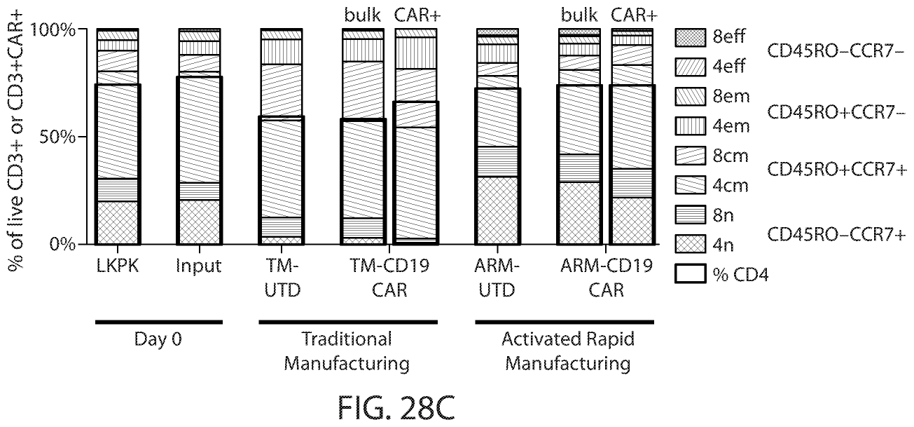

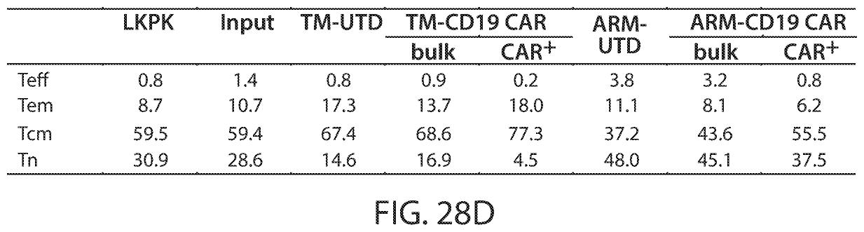

To test the ARM process for anti-CD19 CAR T cell manufacturing at clinical scale, a frozen healthy donor leukapheresis product (Leukopak, LKPK) was used as starting material, described in FIG. 28A as a representative example. The LKPK contained 37% T cells, 4% NK cells, 37% monocytes and 15% B cells (FIG. 28A). After thawing, T cells were positively selected using anti-CD4 and anti-CD8 microbeads. The composition of the product after positive T cell selection was 95.4% T cells, 1.9% NK cells, 1.7% monocytes, and 0.1% B cells (FIG. 28A).

Positively selected T cells were activated using a polymeric nanomatrix conjugated to anti-CD3 and anti-CD28 agonist monoclonal antibodies and transduced with a lentiviral vector encoding anti-CD19 CAR. After 24 hours in culture, cells were harvested and cryo-preserved (such cells are referred to as “ARM-CD19 CAR” in this example). In parallel, CAR-T cells were generated using a traditional manufacturing (TM) process (such cells are referred to as “TM-CD19 CAR” in this example), using the same donor T cells and lentiviral vector. The TM process utilized paramagnetic beads coupled to anti-CD3 and anti-CD28 antibodies and a 9-day culture period in tissue-culture flasks, followed by the same harvest and freezing procedure. CAR-T cells generated by each process were analyzed by flow cytometry to evaluate CAR expression post thaw, as well as the Tcell phenotype (FIGS. 28B-28D). Analysis of the T-cell phenotype revealed that the ARM process retained naïve-like T cells (45.1% CD45RO−/CCR7+) in both the CD8 and CD4 compartments, while the TM process mainly resulted in central-memory T (TCM) cells (68.6% CD45RO+/CCR7+ compared to 43.6% for ARM-CD19 CAR) (FIGS. 28C and 28D). Importantly, the ARM process better maintained the initial naïve-like CD45RO−/CCR7+ T-cell population as compared to the TM process, also in the CAR+ population (28.6% in starting material, 37.5% for ARM-CD19 CAR and 4.5% for TM-CD19 CAR) (FIGS. 28C and 28D). This T-cell population largely overlaps with CD45RO−/CD27+ Tstem cells described by Fraietta, et al (2018) Nat Med, 24 (5); 563-571 and associated with sustained remission in a CLL phase I clinical trial.

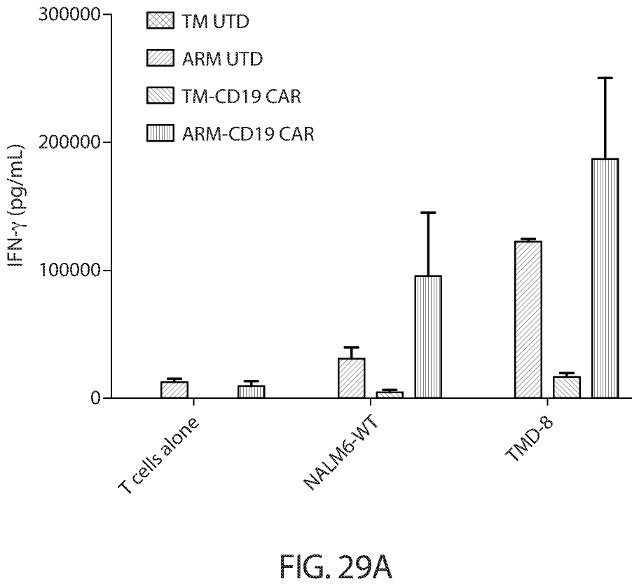

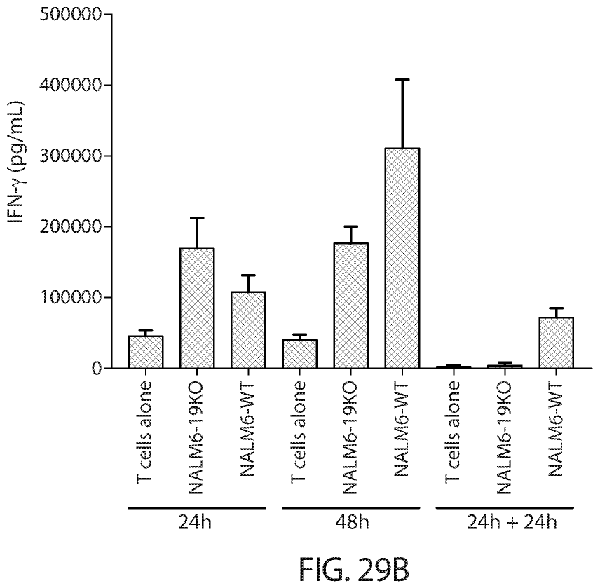

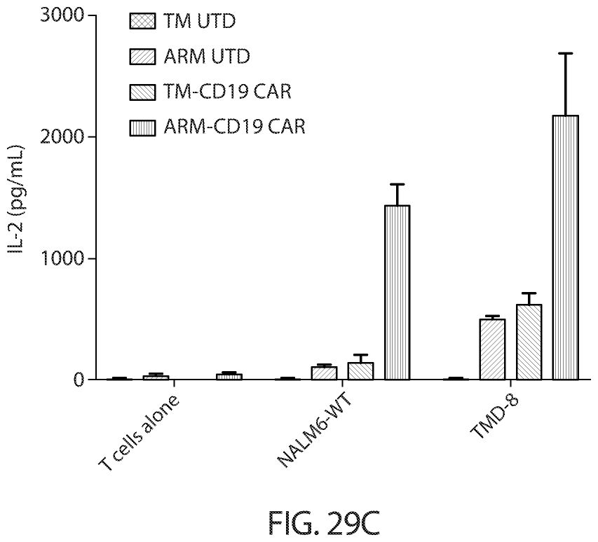

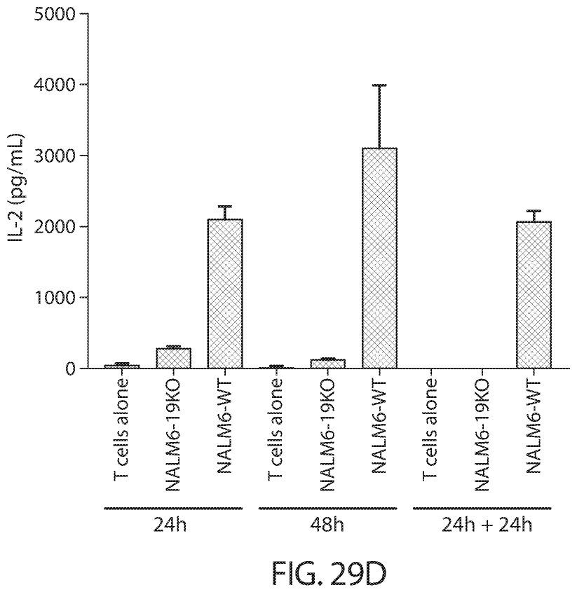

In addition to its phenotype, the final ARM-CD19 CAR cell product was also assessed for its function in vitro. ARM-CD19 CAR and TM-CD19 CAR were thawed and co-cultured with the CD19-expressing cell lines NALM6 (ALL) or TMD-8 (DLBCL). Comparison of cytokine levels in the supernatants 48 hours after co-culture revealed a 11- to 17-fold increase of IFN-γ and a 3.5- to 10-fold increase in levels of IL-2 secreted by ARM-CD19 CAR as compared to TM-CD19 CAR, depending on the stimulating cancer cells (NALM6 or TMD-8, FIGS. 29A and 29C). Experiments with untransduced (UTD) cells that underwent the ARM or TM process (FIG. 29C), or with CD19-negative NALM6 (NALM6-19KO) target cells (FIG. 29D) confirmed CD19-specific recognition by ARM-CD19 CAR and TM-CD19 CAR. Higher background of IFN-γ secretion by ARM-UTD and ARM-CD19 CAR in the absence of CD19-specific stimulation (FIGS. 29A and 29B, respectively) is likely due to the activated nature of these products. This background secretion decreased by 48 hours of coculture (FIGS. 29B and 29D). An intermediate wash of the cells after the first 24 hours of coculture with target cells, followed by co-culture for additional 24 hours (24 h+24 h) further enhanced the difference between background and CD19-specific cytokine secretion. This 24 h+24 h condition highlights that background IFN-γ secretion by ARM-CD19 CAR abates after the first 24 hours.

In summary, the ARM process used to generate ARM-CD19 CAR results in T cells with CAR-expression similar or higher than that of TM-CD19 CAR. Importantly, the ARM process maintains a T-cell phenotype similar to the input material. ARM-CD19 CAR demonstrates CD19-specific activation in vitro, and secretes higher levels of IL-2 as compared to TM-CD19 CAR, which correlates with its Tₛₜₑₘ phenotype.

### In Vivo Efficacy

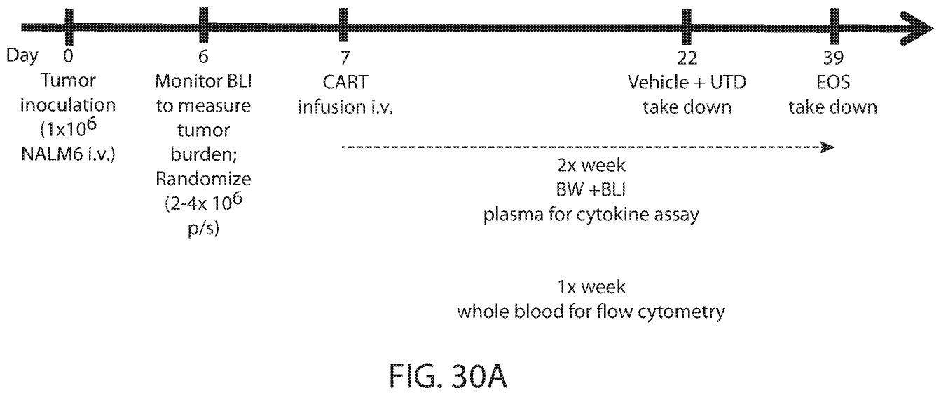

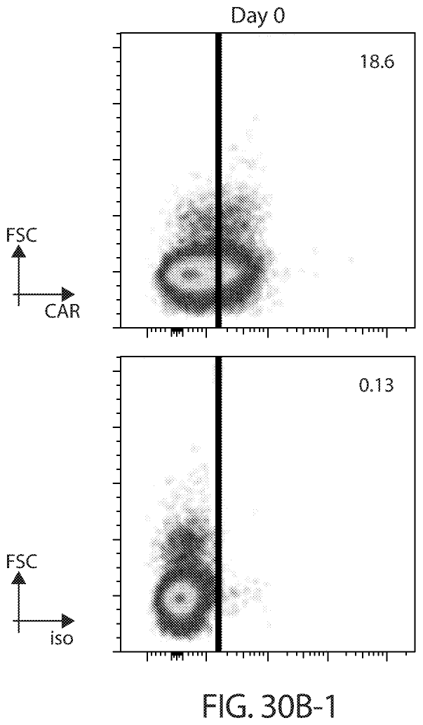

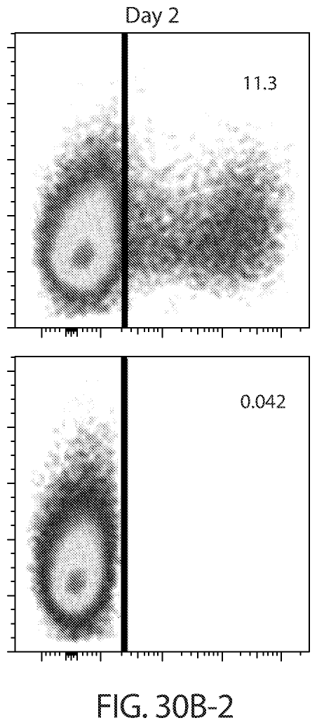

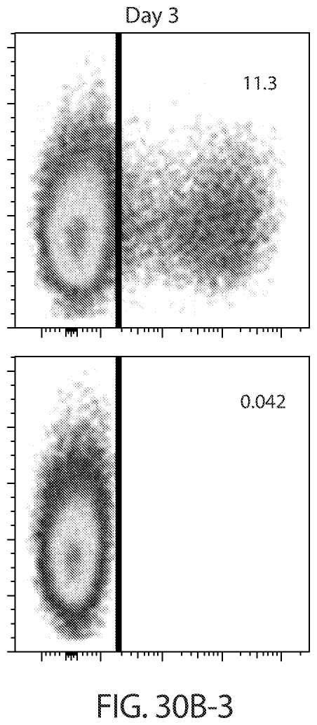

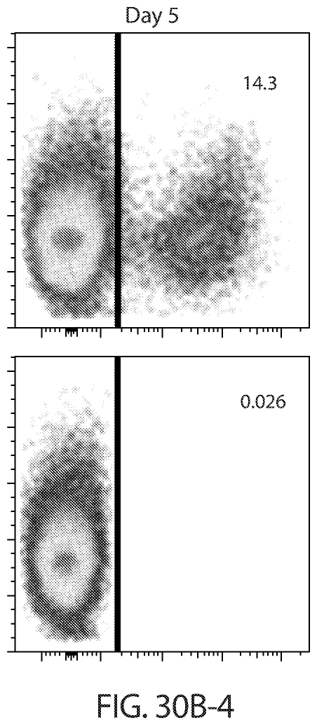

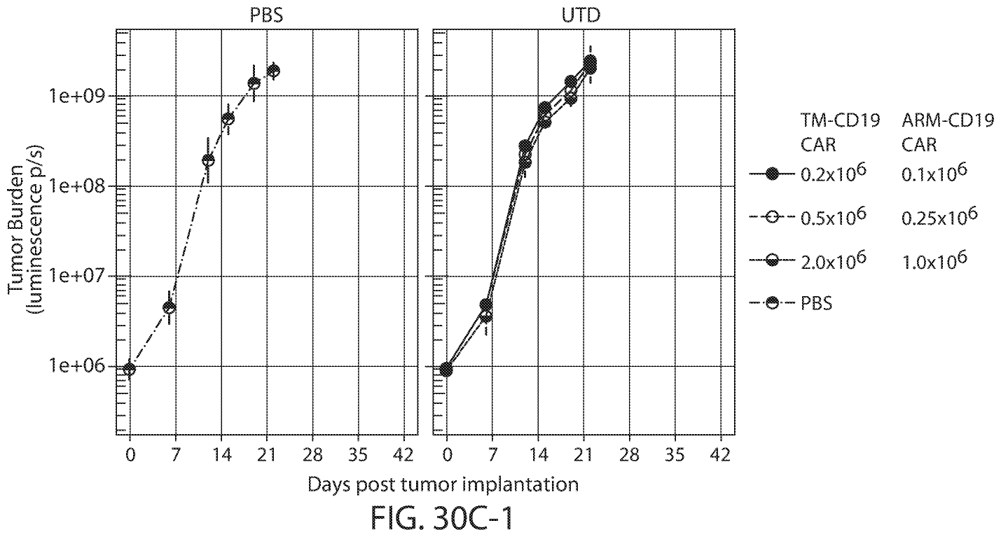

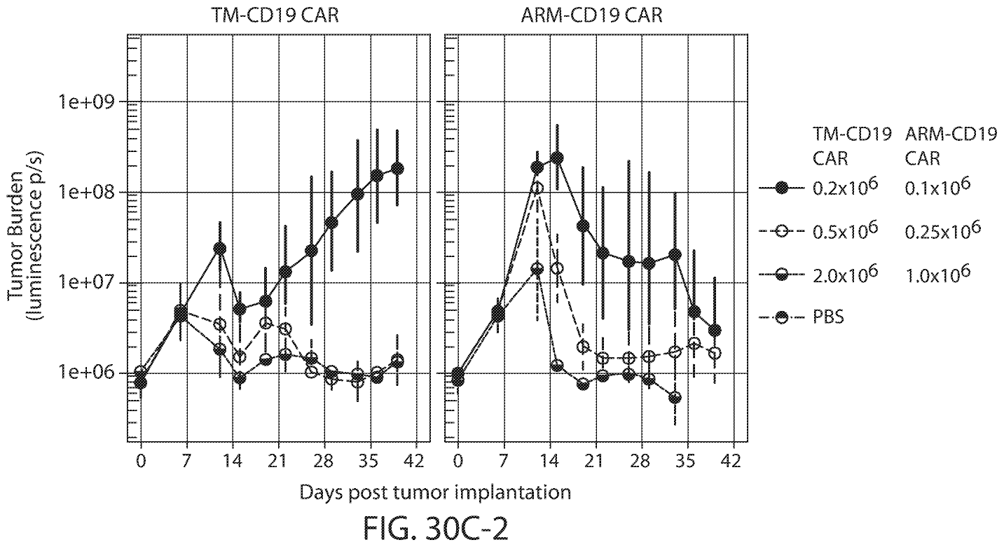

Efficacy studies in vivo were used to guide the development of the ARM process, ultimately leading to the process that will be used for clinical anti-CD19 CAR T cell manufacturing. For the experiment described here, ARM-CD19 CAR was generated at clinical scale. In parallel, TM-CD19 CAR was generated using the same lentiviral vector and T cells from the same donor. The efficacy of CAR-T cells generated using the different processes was evaluated in immunodeficient NSG mice (NOD-scid IL2Rg-null), which were inoculated with the pre-B ALL cell line NALM6. This tumor cell line engrafts in the bone marrow, but in case of high tumor burdens can also be detected in the circulation. Seven days after leukemia inoculation, cohorts of mice received a single infusion of CAR+ T cells (FIG. 30A). Planned doses of 0.2×10⁶, 0.5×10⁶ and 2×10⁶ viable CAR+ T cells were determined based on post thaw flow analysis of TM-CD19 CAR and ARM-CD19 CAR on day 0.

Because of the concern of pseudo-transduction for ARM-CD19 CAR on day 0 post thaw, a sentinel vial was thawed and cultured for up to 5 days, and CAR expression (percentage and mean fluorescence intensity) was analyzed by flow cytometry at different time points (FIG. 30B). The percentage of positive cells on later time points was lower as compared to the day 0 post-thaw sample. At the same time, CAR mean fluorescence intensity was higher per cell, reflective of stably transduced CAR-T cells. The measurement on day 3 was used to determine the actual dose of ARM-CD19 CAR, which was determined to be 0.1×10⁶, 0.25×10⁶ and 1×10⁶ viable CAR+ T cells. The TM-CD19 CAR dose remained unchanged (0.2×10⁶, 0.5×10⁶ and 2×10⁶ viable CAR+ T cells), as the flow analysis of post-thaw samples was performed on rested, fully integrated CART cells.

Both ARM-CD19 CAR and TM-CD19 CAR induced tumor-regression in a dose-dependent manner (FIG. 30C). Mice treated with 0.5×10⁶ or 2×10⁶ TM-CD19 CAR cells, or 0.25×10⁶ or 1×10⁶ ARM-CD19 CAR cells, experienced durable tumor regression. Interestingly, at the respective lowest dose tested (0.2×10⁶ TM-CD19 CAR cells or 0.1×10⁶ ARM-CD19 CAR cells), response to TM-CD19 CAR was not sustained and all mice eventually relapsed after initial partial leukemia control. In contrast, at the lowest dose (0.1×10⁶) ARM-CD19 CAR-treated mice showed a steady decline of tumor burden that lasted until the end of study. The kinetics of tumor regression suggest a delayed activation of ARM-CD19 CAR by about 1 week, suggesting that Tₛₜₑₘ cells need to proliferate and differentiate into effector cells in order to exert their antitumor activity.

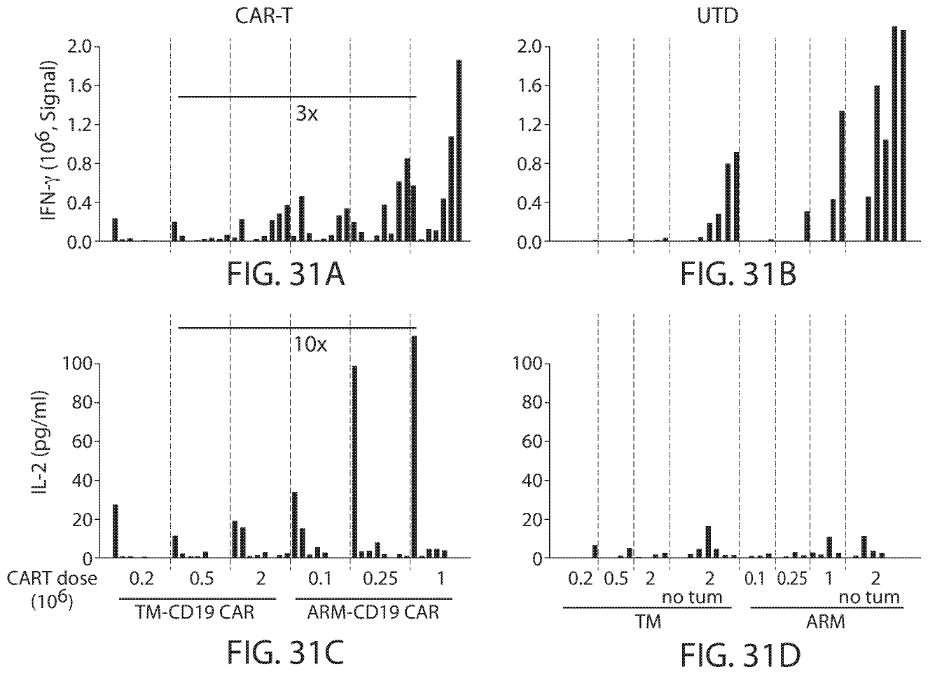

Mice treated with CAR-T cells and UTD cells generated by the two manufacturing processes were bled twice weekly to measure cytokine levels (FIGS. 31A-31D). Circulating IFN-γ levels in mice infused with CAR-T cells, either ARM-CD19 CAR or TM-CD19 CAR, showed a bi-phasic pattern (FIG. 31A). An early IFN-γ peak was observed at days 4-7 after CAR-T cell infusion and likely related to CD19-specific activation following tumor recognition, since it was not evident in mice infused with TM-UTD or ARM-UTD (FIG. 31B). Early CD19-mediated activation was confirmed by a concomitant rise of in vivo IL-2 levels (FIG. 31C), which however abated at later time points.

### In Vivo Cellular Kinetics

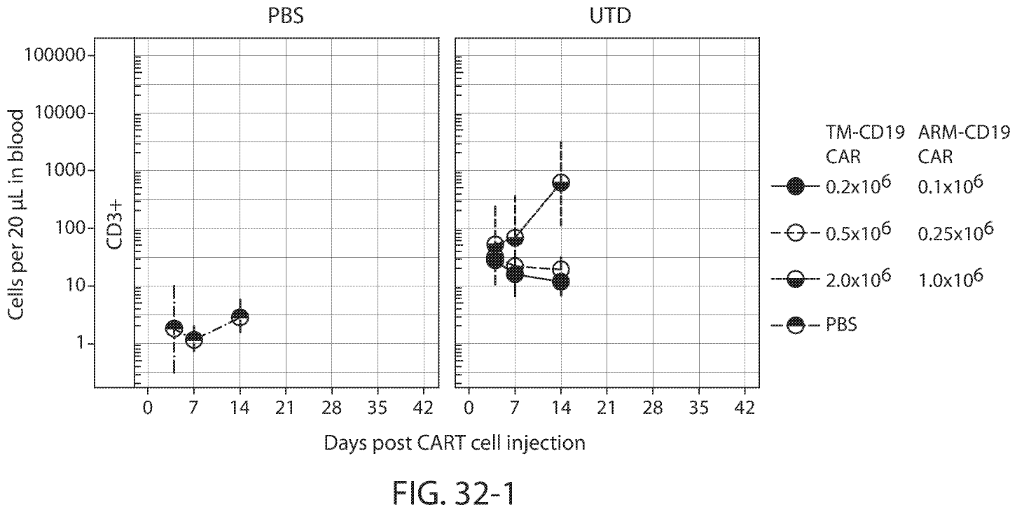

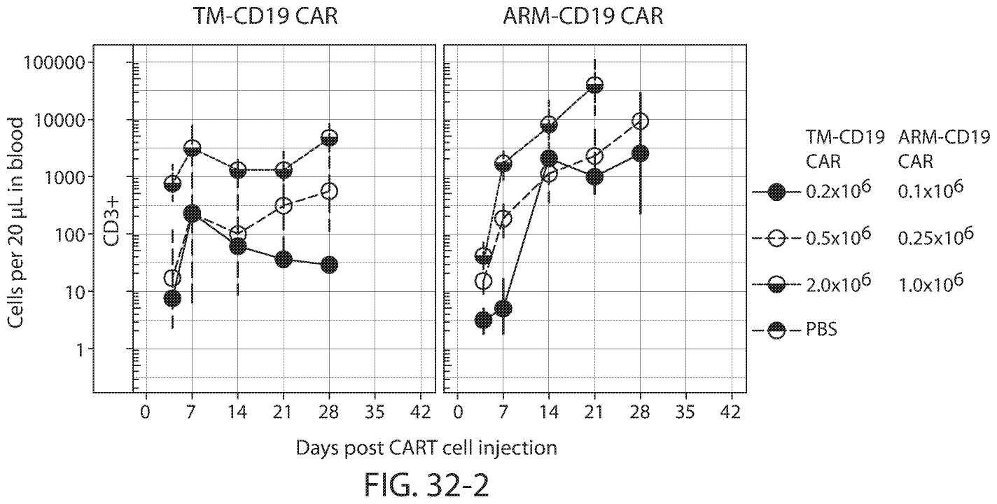

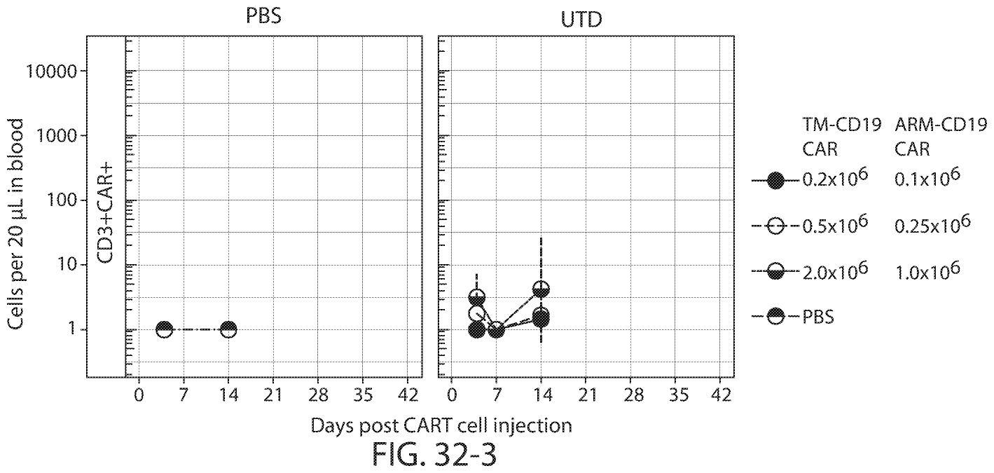

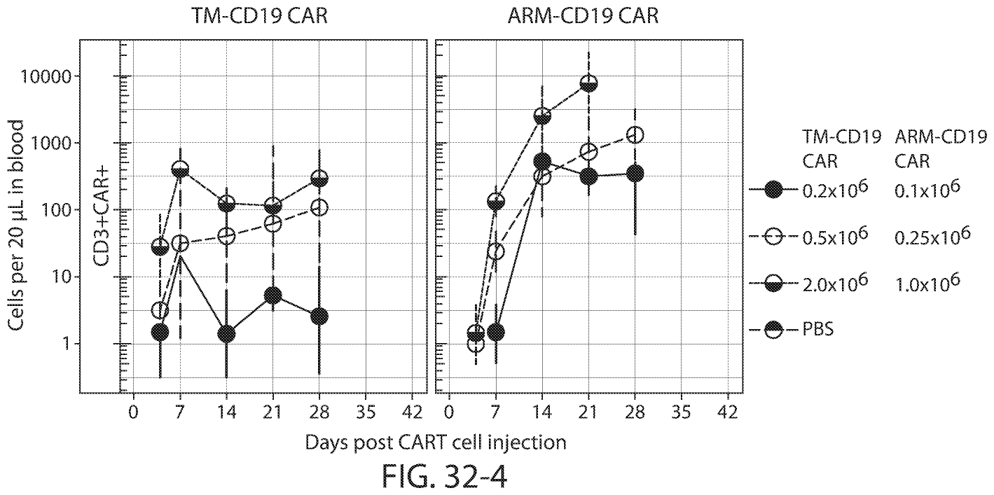

As part of a pharmacology study to evaluate the efficacy of ARM-CD19 CAR in NSG mice, the expansion of CAR+ T cells was assessed in vivo (FIG. 32). CD3+/CAR+ T-cell concentration in blood was analyzed by flow cytometry up to 4 weeks after infusion. CAR-T cell expansion can be inferred. However, long-term persistence cannot be assessed due to limited study time dictated by onset of X-GVHD. Cellular expansion was observed for both ARM-CD19 CAR and TM-CD19 CAR at all doses, except for TM-CD19 CAR at the lowest dose of 0.2×10⁶ cells. Exposure (Cmax and AUC within 21 days post cell injection) increased with increasing dose for both TM-CD19 CAR and ARM-CD19 CAR. To compare the expansion of ARM-CD19 CAR to TM-CD19 CAR at the same dose level, exposure of TM-CD19 CAR was interpolated to comparable doses of ARM-CD19 CAR (0.25×10⁶ and 1×10⁶ cells). The Cmax was 24- to 46-times higher and the AUC0-21d was 18- to 33-times higher compared to TM-CD19 CAR at doses of 0.25×10⁶ and 1×10⁶ cells. The time to ARM-CD19 CAR peak expansion (Tmax) was delayed for at least 1 week compared to TM-CD19 CAR.

In summary, pharmacology studies evaluating ARM-CD19 CAR in vitro show that ARM-CD19 CAR has an early-differentiated phenotype and has the potential to secrete more IFN-γ and IL-2. In vivo, ARM-CD19 CAR demonstrated delayed but higher cellular expansion, induced more IL-2 secretion, and controlled tumor growth at lower doses as compared to TM-CD19 CAR. Other features of ARM-CD19 CAR discussed, such as elevated levels of plasma IFN-γ at later time points and earlier occurrence of X-GVHD were seen both for ARM-CD19 CAR, as well as for ARM-UTD, underlying the limitations of the xenograft mouse model used here. Together, these results support the hypothesis that ARM-CD19 CAR contains T cells with more stemness features, enabling ARM-CD19 CAR to effectively engraft, expand and reject tumors.

### In Vitro IL-6 Release Assay

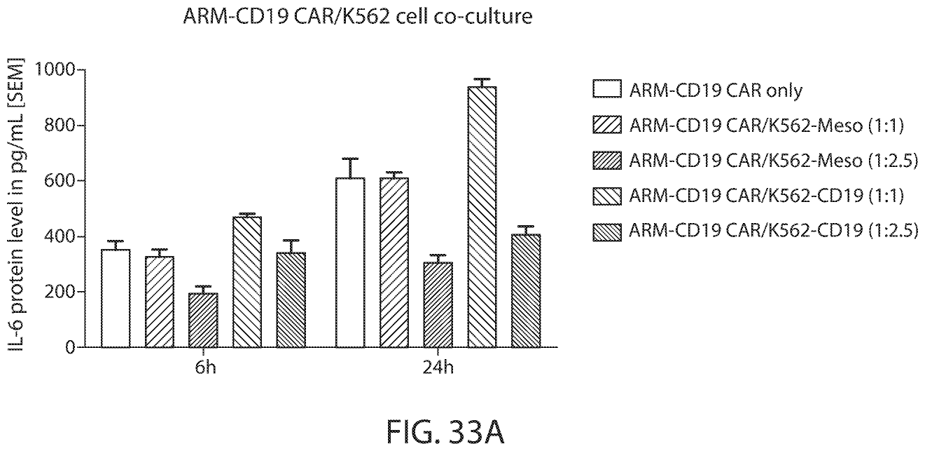

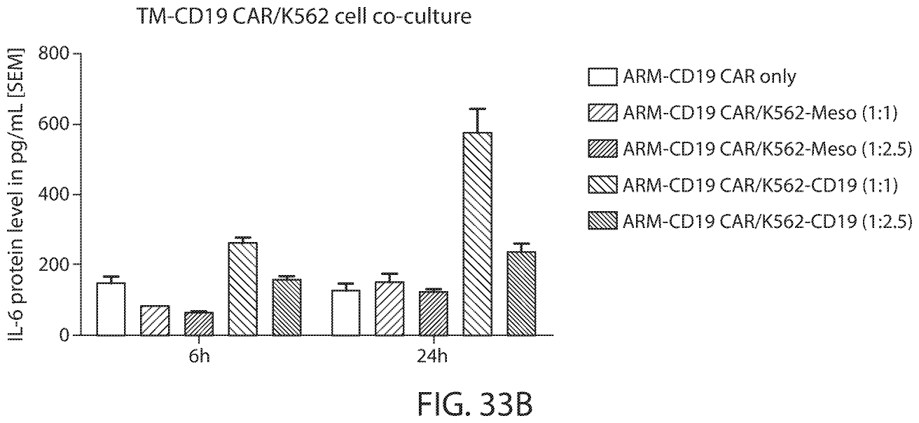

A three-party co-culture model for the in vitro investigation of IL-6 induction potential by CART cells was first published by Norelli, et al (2018) Nat Med., June; 24 (6); 739-748 and applied here with some adaptations. This model consists of CAR-T cells, leukemic target cells and bystander THP-1 monocytic cells, as a source of myeloid cells for maximized IL-6 production. In this in vitro cellular model, IL-6 secretion by either ARM-CD19 CAR or TM-CD19 CAR alone was increased by co-culturing with CD19-expressing targets and THP-1 cells (FIGS. 33A and 33B). Importantly, time-dependent CD19-specific IL-6 secretion induced by ARM-CD19 CAR was superimposable to that induced by TM-CD19 CAR. In the same in vitro model, CD19-specific IFN-γ secretion in the ARM-CD19 CAR condition was 10-fold higher than in the TM-CD19 CAR condition (data not shown).

### Summary

These results suggest that ARM-CD19 CAR might have greater antitumor potential and a similar safety profile as compared to TM-CD19 CAR. Greater antitumor potential is inferred by better tumor control at the lowest dose tested and by higher in vivo cellular expansion. Such a calculation may however be an underestimation of the overall therapeutic potential of ARM-CD19 CAR, since this was assayed in an ALL model (NALM6) which is more aggressive than the two disease indications (CLL and DLBCL) in which ARM-CD19 CAR will be initially investigated. In CLL, in particular, where in vivo CAR-T cell expansion robustly correlates with tumor regression (Mueller, et al (2017) Blood. 130 (21); 2317-2325; Fraietta, et al (2018) Nat Med, 24 (5); 563-571), significantly higher proliferative potential of ARM-CD19 CAR (up to 20-fold) might result in meaningful superior efficacy compared to TM-CD19 CAR. In mice, the early systemic release of IFN-γ and IL-2 by ARM-CD19 CAR associated with CAR-mediated tumor regression was 3-fold and 10-fold higher than that induced by traditionally manufactured CAR-T cells, respectively. IL-6 levels were not studied in vivo, since in this strain lack of functional myeloid cells results in the inability to produce inflammatory cytokines (Norelli, et al (2018) Nat Med., June; 24 (6); 739-748; Giavridis, et al (2018) Nat Med., June; 24 (6); 731-738). To obviate this and evaluate the potential for in vivo IL-6 release induced by ARM-CD19 CAR, an in vitro three-party co-culture system was employed, in which bystander monocytic cells are added as a source of inflammatory cytokines (Norelli, et al (2018) Nat Med., June; 24 (6); 739-748). In this system, IL-6 production was similar between ARM-CD19 CAR and traditionally manufactured CAR-T cells, suggesting a similar risk for CRS. Conversely, the delayed kinetics of ARM-CD19 CAR cellular expansion will require an extension of the CRS monitoring period from the 3 weeks typical of TM-CD19 CAR, to 4 weeks. In vitro experiments with ARM-CD19 CAR also revealed the potential for transient, non-CAR-mediated IFN-γ and IL-2 secretion by ARM-CD19 CAR during the first 3 days of culture after thawing. A comprehensive risk assessment based on data from patients receiving recombinant human IL-2 (Proleukin) and recombinant human IFN-γ (ACTIMMUNE), and taking in consideration the projected exposures following ARM-CD19 CAR infusion indicates that the risk for constitutional symptoms (fever, chills, erythema) as described in these patients, would be very low. To further mitigate this risk, patients receiving ARM-CD19 CAR will be hospitalized for at least 72 hours after infusion of the cellular product.

Finally, in the non-GLP compliant toxicology study, NSG mice engrafted with ARM-CD19 CAR did not show unexpected behavior in comparison to traditionally manufactured CAR-T cells and untransduced cells undergoing the ARM process, when assessed by blood or lymphatic organ immunophenotyping, as well as histological evaluation of a relevant set of organs.

### Example 9: BCMA CART Cells Manufactured Using the ARM Process

### Methods

### T Cell Isolation

Fresh leukopak of healthy donor aphereses were obtained from Hemacare and stored in vapor phase liquid nitrogen (LN2) until needed. On Day 0, two quarter leukopaks were removed from LN2, warmed in the Plasmatherm (Barkey, Leopoldshöhe, Germany) until a small ice crystal remained, and diluted with Prodigy® process buffer. Automated CD4/CD8 positive selection was then performed on the CliniMACS® Prodigy® with the TS 520 tubing set and T Cell Transduction (TCT) program software version 1.0. Cell count and viability for each Prodigy® output (product, waste, and nontarget cells) were determined by AO/PI staining as enumerated by the Cellometer Vision (Nexcelom, Lawrence, MA) to assess total cell recovery and T cell recovery. The CD4/CD8-enriched product was eluted in OpTmizer™ complete T cell medium and divided for further culturing using either the 24 h or traditional 9-day process (TM). Remaining T cells were frozen down in LN tank. T cell purity was evaluated by flow cytometry analyses.

### CAR-T Cells Production Using the ARM Process

T cells purified by Prodigy® were seeded into different scales of vessels, such as plate, flask, G-REX vessel or full clinical scale in centricult. Upon seeding, TransAct (Miltenyi Biotec)), a polymeric nanomatrix conjugated to anti-CD3 and anti-CD28 agonist, was added, in addition to clinical-grade lentiviral vector. Cells were incubated in OpTmizer™ complete T cell media containing 100 IU/mL human recombinant IL-2 (Prometheus, San Diego, CA), 2% ICRS (Life Technologies) for 24 h prior to harvest and cryopreservation.

Aliquots of cryopreserved CAR-T cells were thawed into pre-warmed OpTmizer™ complete media, washed twice with 20× volume of pre-warmed medium before culturing and flow cytometry analyses for assessing BCMA-CAR expression and stemness features at different time points post-thaw. Aliquots of the cell products were co-cultured with target cell lines to assess cytokine release in response to specific antigen stimulation.

### CAR-T Cells Production Using TM Process

Prodigy® processed T cells were resuspended in warm RPMI complete T cell medium and plated in 24-well plates. T cells were incubated overnight at 37° C. with Human T-Expander CD3/CD28 beads at a 3:1 ratio of beads-to-cells.

On Day 1, lentiviruses were added at a MOI of 2, based on the SUP-T1titer. No virus was added to the untransduced control (UTD). The T cells were incubated overnight at 37° C. followed by the addition of 1 mL complete T cell medium per well, after which they were incubated overnight at 37° C. For the remaining seven days of culture expansion, the T cells were transferred into tissue culture flasks and diluted with complete T cell medium every two days.

Between Days 8 to 9, the T cells were de-beaded, harvested and cryopreserved in CryoStor CS10 freezing medium, frozen at −80° C. in CoolCell Cell Freezing Containers (Biocision), and transferred to LN2 the following day. Small aliquots of T cells were stained for CAR expression. Single color controls were included for compensation. Samples were measured on a flow cytometer (BD LSRFortessa), and data were analyzed with FlowJo software.

### Target Cell Line and Culture

Nalm6 cells were transfected with a lentiviral firefly luciferase reporter construct to create the Nalm6-luc cell line. The cells were grown in incubators at 37° C. with 5% CO₂. An aliquot of cells was used for detection of tumor antigen BCMA expression prior to use.

### In Vitro Cytokine Secretion Assay

Cytokine secretion of anti-BCMA CAR-T (referred to as effector cells) in response to a BCMA-expressing target cell was evaluated by incubating CAR-T cells with target cells at 2.5-fold E: T ratio for 20 h in 96-well flat-bottom plates. Effector cells were PI61, R1G5 and BCMA10 CART cells generated using either the ARM or TM process. CART cells manufactured using the ARM process were plated for a 24 h washout condition to allow the cells to rest and minimize non-specific activity. Target cells include BCMA positive KMS11-luc or BCMA negative NALM6-luc. These target cells were added to the freshly plated T cells or T cells from the 24 h washout condition (ARM cells only). For this assay, the % transduction of CAR-T cells was normalized by addition of UTD to the BCMA CAR-Ts. This allowed for the comparison of the same number of CAR-Ts and same total T cell number in each sample. Supernatants from the 20-hour co-culture time point of effector to target were harvested from each well and frozen at −20° C. to be used for MSD cytokine analysis. The custom MSD V-PLEX Human IFN-γ, IL-2 Kit (#K151A0H-4A) was used to quantify the secreted cytokines in each of the supernatant samples.

### Results

### ARM Process Preserves T Cell Stemness

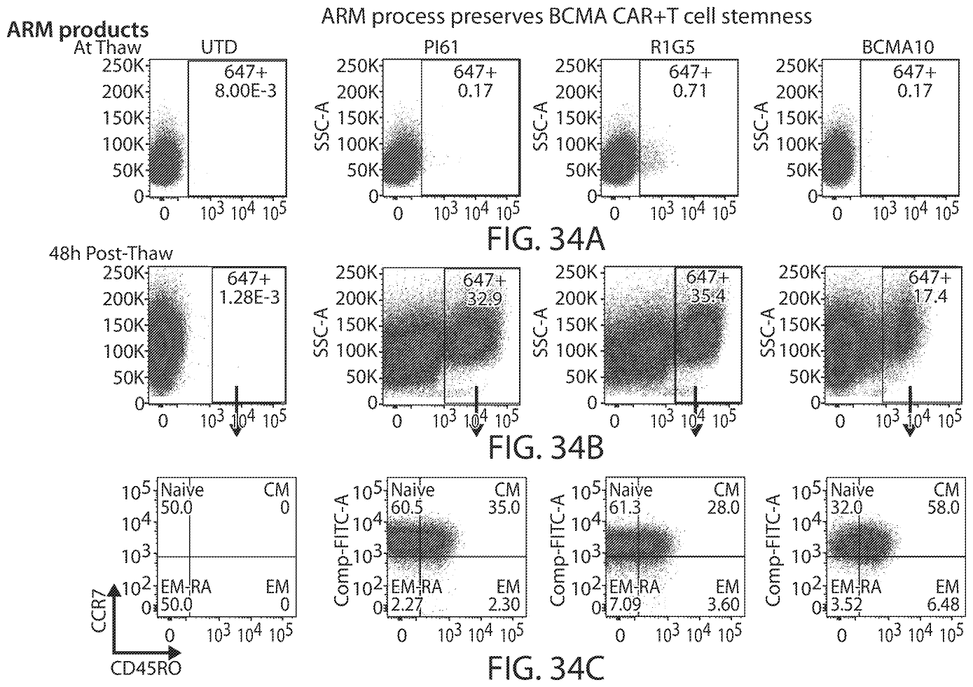

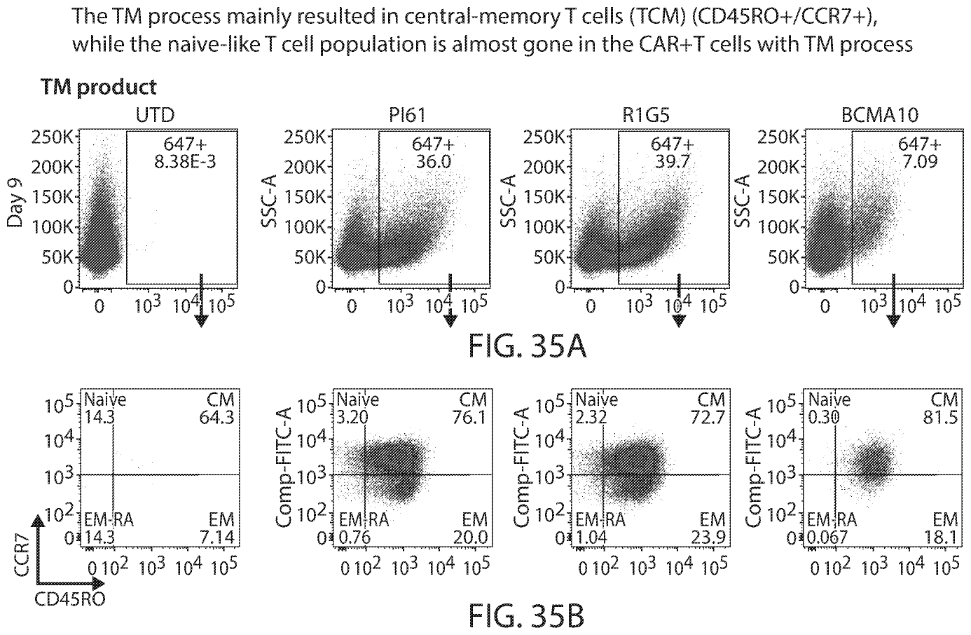

CAR-T cells generated using the ARM process were analyzed by flow cytometry to evaluate their CAR expression at thaw and 48 h post thaw, as well as the T-cell phenotype (FIGS. 34A, 34B, and 34C). For CAR-T cells manufactured using the TM process, CAR expression was assessed at day 9 before harvest (FIG. 35A). BCMA-CAR was almost undetectable at thaw shown in FIG. 34A. However, at 48 h post-thaw, BCMA-CAR was clearly being expressed as 32.9% for PI61, 35.9% for R1G5 and 17.4% for BCMA10. The day 9 cells generated using the TM process show BCMA-CAR expression to be 36% for PI61, 40% for R1G5 and 7% for BCMA10 (FIG. 35A). Analysis of the CAR+ T-cell phenotype revealed that the ARM process retained naïve-like T cells (˜60% of CD45RO−/CCR7+ for PI61 and R1G5, 32% of CD45RO−/CCR7+ for BCMA10) (FIG. 34C). The TM process mainly resulted in central-memory T cells (TCM) (72˜81% CD45RO+/CCR7+ for all three BCMA CAR-Ts), while the naive-like T cell population was almost gone in the CAR+ T cells manufactured using the TM process (FIG. 35B). Overall, the naïve T-cell population largely overlaps with CD45RO−/CD27+ Tstem cells described by previous reports (Cohen A D, et al (2019). J Clin Invest. 130. pii: 126397. doi: 10.1172/JCI126397; Fraietta, J A, et al (2018). Nat Med, 24 (5); 563-571) and is associated with responses and CAR-T expansion.

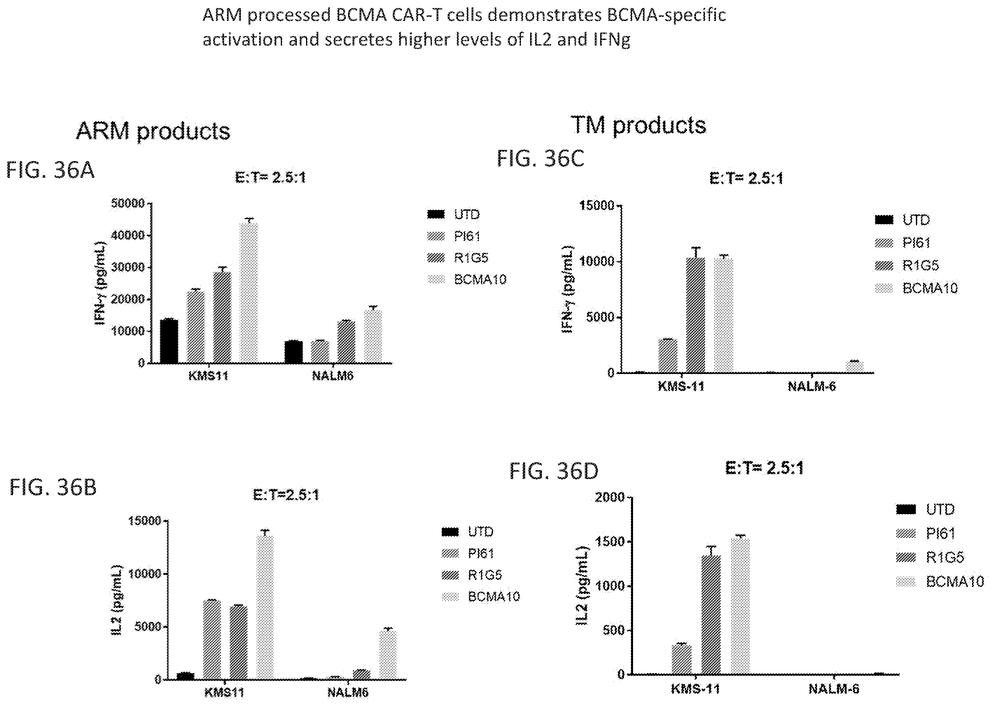

In addition to its phenotype, the final PI61, R1G5 and BCMA10 CART cell products were also assessed for their function in vitro. PI61, R1G5 and BCMA10 cell products were thawed and co-cultured with the BCMA-expressing cell line KMS-11 at 1:1 ratio. Post-thaw ARM processed cells were rested for 24 h prior to co-culture being established. Comparing cytokine levels in the supernatants 24 hours after co-culture revealed a ˜5 to 25-fold increase of IL-2 and a ˜3 to 7-fold increase in levels of IFN-γ secreted by ARM products as compared to TM products as shown in FIGS. 36A-36D. Experiments with untransduced (UTD) cells that underwent the ARM or TM process confirmed BCMA-specific recognition by PI61, R1G5 and BCMA10.

In summary, PI61, R1G5 and BCMA10 CART cells produced using the ARM process demonstrate BCMA-specific activation in vitro and secretes higher levels of IL-2 and IFN-γ as compared to TM processed products, which correlates with the Tₛₜₑₘ phenotype of CART cells produced using the ARM process.

### Example 10: Gene Signature Analysis of CART Cells Manufactured Using the ARM Process

### Methods

### Single Cell RNAseq

Single cell RNAseq libraries were generated using the 10× Genomics Chromium Controller instrument and supporting library construction kits.

Cryopreserved cells were thawed, counted and flow sorted (if required for study question), prior to being loaded on a 10× Genomics Instrument. Individual cells were loaded into droplets and RNA within individual droplets was barcoded via a GemCode bead. Barcoded RNA was released from droplets and converted into a whole transcriptome Illumina compatible sequencing library.

Generated libraries were sequenced on an Illumina HiSeq Instrument and analyzed using 10× Genomics analysis pipeline and Loupe Cell Browser software.

### Single Cell Immune Cell Profiling

Whole transcriptome 10× Genomics single cell libraries were used as a template material to generate immune cell profiling and repertoire analysis. T cell receptor sequences were PCR amplified from Chromium Single Cell 5′ Libraries and analyzed on an Illumina sequencing instrument.

### Analysis Pipeline

Single cell RNAseq data was processed through the Cell Ranger analysis pipeline starting with FASTQ files. A detailed description of the Cell Ranger analysis pipeline can be found at: https://support. 10×genomics.com/single-cell-gene-expression/software/pipelines/latest/what-is-cell-ranger. The general pipeline included alignment, filtering, barcode counting, and UMI counting. Cellular barcodes were used to generate gene-barcode matrices, determine clusters, and perform gene expression analysis. Gene expression count data was normalized using the Seurat Bioconductor package. Cells were discarded from the analysis that had less than 200 expressed genes. Genes were discarded from the analysis that were only expressed in 2 cells or less. The remaining data was normalized with the Seurat log normalization method using a scale factor of 10,000. Data was scaled by regressing on the number of detected molecules per cell. The gene set score (GeneSetScore) was calculated by taking the mean log normalized gene expression value of all the genes in the gene set. Each gene is z-score normalized so that the mean expression of the gene across samples is 0 and standard deviation is 1. The gene set score is then calculated as the mean of the normalized values of the genes in the gene set. An exemplary gene set score calculation is described below.

For this example of gene set score calculation, the normalized gene expression of two (2) samples for six (6) genes is provided in Table 23. For the purposes of this exemplary calculation, the gene set consists of genes 1-4. Therefore, Sample 1 and 2 both have gene set scores of 0.

TABLE 23
Exemplary dataset for gene set score calculation

| | Sample 1 | Sample 2 |
|---|---|---|
| Gene 1 | −3 | 0 |
| Gene 2 | 3 | 0 |
| Gene 3 | 1 | 0 |
| Gene 4 | −1 | 0 |
| Gene 5 | 10 | 4 |
| Gene 6 | −5 | 3 |

The gene set “Up TEM vs. Down TSCM” includes the following genes: MXRA7, CLIC1, NAT13, TBC1D2B, GLCCI1, DUSP10, APOBEC3D, CACNB3, ANXA2P2, TPRG1, EOMES, MATK, ARHGAP10, ADAM8, MAN1A1, SLFN12L, SH2D2A, EIF2C4, CD58, MYO1F, RAB27B, ERN1, NPC1, NBEAL2, APOBEC3G, SYTL2, SLC4A4, PIK3AP1, PTGDR, MAF, PLEKHA5, ADRB2, PLXND1, GNAO1, THBS1, PPP2R2B, CYTH3, KLRF1, FLJ16686, AUTS2, PTPRM, GNLY, and GFPT2.

The gene set “Up Treg vs. Down Teff” includes the following genes: C12orf75, SELPLG, SWAP70, RGS1, PRR11, SPATS2L, SPATS2L, TSHR, C14orf145, CASP8, SYT11, ACTN4, ANXA5, GLRX, HLA-DMB, PMCH, RAB11FIP1, IL32, FAM160B1, SHMT2, FRMD4B, CCR3, TNFRSF13B, NTNG2, CLDND1, BARD1, FCER1G, TYMS, ATP1B1, GJB6, FGL2, TK1, SLC2A8, CDKN2A, SKAP2, GPR55, CDCA7, S100A4, GDPD5, PMAIP1, ACOT9, CEP55, SGMS1, ADPRH, AKAP2, HDAC9, IKZF4, CARD17, VAV3, OBFC2A, ITGB1, CIITA, SETD7, HLA-DMA, CCR10, KIAA0101, SLC14A1, PTTG3P, DUSP10, FAM164A, PYHIN1, MYO1F, SLC1A4, MYBL2, PTTG1, RRM2, TP53INP1, CCR5, ST8SIA6, TOX, BFSP2, ITPRIPL1, NCAPH, HLA-DPB2, SYT4, NINJ2, FAM46C, CCR4, GBP5, C15orf53, LMCD1, MKI67, NUSAP1, PDE4A, E2F2, CD58, ARHGEF12, LOC100188949, FAS, HLA-DPB1, SELP, WEE1, HLA-DPA1, FCRL1, ICA1, CNTNAP1, OAS1, METTL7A, CCR6, HLA-DRB4, ANXA2P3, STAM, HLA-DQB2, LGALS1, ANXA2, PI16, DUSP4, LAYN, ANXA2P2, PTPLA, ANXA2P1, ZNF365, LAIR2, LOC541471, RASGRP4, BCAS1, UTS2, MIAT, PRDM1, SEMA3G, FAM129A, HPGD, NCF4, LGALS3, CEACAM4, JAKMIP1, TIGIT, HLA-DRA, IKZF2, HLA-DRB1, FANK1, RTKN2, TRIB1, FCRL3, and FOXP3.

The gene set “Down stemness” includes the following genes: ACE, BATF, CDK6, CHD2, ERCC2, HOXB4, MEOX1, SFRP1, SP7, SRF, TAL1, and XRCC5.

The gene set “Up hypoxia” includes the following genes: ABCB1, ACAT1, ADM, ADORA2B, AK2, AK3, ALDH1A1, ALDH1A3, ALDOA, ALDOC, ANGPT2, ANGPTL4, ANXA1, ANXA2, ANXA5, ARHGAP5, ARSE, ART1, BACE2, BATF3, BCL2L1, BCL2L2, BHLHE40, BHLHE41, BIK, BIRC2, BNIP3, BNIP3L, BPI, BTG1, C11orf2, C7orf68, CA12, CA9, CALD1, CCNG2, CCT6A, CD99, CDK1, CDKN1A, CDKN1B, CITED2, CLK1, CNOT7, COL4A5, COL5A1, COL5A2, COL5A3, CP, CTSD, CXCR4, D4S234E, DDIT3, DDIT4, 1-Dec, DKC1, DR1, EDN1, EDN2, EFNA1, EGF, EGR1, EIF4A3, ELF3, ELL2, 20) ENG, ENO1, ENO3, ENPEP, EPO, ERRFI1, ETS1, F3, FABP5, FGF3, FKBP4, FLT1, FN1, FOS, FTL, GAPDH, GBE1, GLRX, GPI, GPRC5A, HAP1, HBP1, HDAC1, HDAC9, HERC3, HERPUD1, HGF, H1F1A, HK1, HK2, HLA-DQB1, HMOX1, HMOX2, HSPA5, HSPD1, HSPH1, HYOU1, ICAM1, ID2, IFI27, IGF2, IGFBP1, IGFBP2, IGFBP3, IGFBP5, IL6, IL8, INSIG1, IRF6, ITGA5, JUN, KDR, KRT14, KRT18, KRT19, LDHA, LDHB, LEP, LGALS1, LONP1, LOX, LRP1, MAP4, MET, MIF, MMP13, MMP2, MMP7, MPI, MT1L, MTL3P, MUC1, MXI1, NDRG1, NFIL3, NFKB1, NFKB2, NOS1, NOS2, NOS2P1, NOS2P2, NOS3, NR3C1, NR4A1, NT5E, ODC1, P4HA1, P4HA2, PAICS, PDGFB, PDK3, PFKFB1, PFKFB3, PFKFB4, PFKL, PGAM1, PGF, PGK1, PGK2, PGM1, PIM1, PIM2, PKM2, PLAU, PLAUR, PLIN2, PLOD2, PNN, PNP, POLM, PPARA, PPAT, PROK1, PSMA3, PSMD9, PTGS1, PTGS2, QSOX1, RBPJ, RELA, RIOK3, RNASEL, RPL36A, RRP9, SATI, SERPINB2, SERPINE1, SGSM2, SIAH2, SIN3A, SIRPA, SLC16A1, SLC16A2, SLC20A1, SLC2A1, SLC2A3, SLC3A2, SLC6A10P, SLC6A16, SLC6A6, SLC6A8, SORL1, SPP1, SRSF6, SSSCA1, STC2, STRA13, SYT7, TBPL1, TCEAL1, TEK, TF, TFF3, TFRC, TGFA, TGFB1, TGFB3, TGFBI, TGM2, TH, THBS1, THBS2, TIMM17A, TNFAIP3, TP53, TPBG, TPD52, TPI1, TXN, TXNIP, UMPS, VEGFA, VEGFB, VEGFC, VIM, VPS11, and XRCC6.

The gene set “Up autophagy” includes the following genes: ABL1, ACBD5, ACIN1, ACTRT1, ADAMTS7, AKR1E2, ALKBH5, ALPK1, AMBRA1, ANXA5, ANXA7, ARSB, ASB2, ATG10, ATG12, ATG13, ATG14, ATG16L1, ATG16L2, ATG2A, ATG2B, ATG3, ATG4A, ATG4B, ATG4C, ATG4D, ATG5, ATG7, ATG9A, ATG9B, ATP13A2, ATP1B1, ATPAF1-AS1, ATPIF1, BECN1, BECN1P1, BLOC1S1, BMP2KL, BNIP1, BNIP3, BOC, C11orf2, C11orf41, C12orf44, C12orf5, C14orf133, C1orf210, C5, C6orf106, C7orf59, C7orf68, C8orf59, C9orf72, CA7, CALCB, CALCOCO2, CAPS, CCDC36, CD163L1, CD93, CDC37, CDKN2A, CHAF1B, CHMP2A, CHMP2B, CHMP3, CHMP4A, CHMP4B, CHMP4C, CHMP6, CHST3, CISD2, CLDN7, CLEC16A, CLN3, CLVS1, COX8A, CPA3, CRNKL1, CSPG5, CTSA, CTSB, CTSD, CXCR7, DAP, DKKL1, DNAAF2, DPF3, DRAM1, DRAM2, DYNLL1, DYNLL2, DZANK1, EI24, EIF2S1, EPG5, EPM2A, FABP1, FAM125A, FAM131B, FAM134B, FAM13B, FAM176A, FAM176B, FAM48A, FANCC, FANCF, FANCL, FBXO7, FCGR3B, FGF14, FGF7, FGFBP1, FIS1, FNBP1L, FOXO1, FUNDC1, FUNDC2, FXR2, GABARAP, GABARAPL1, GABARAPL2, GABARAPL3, GABRA5, GDF5, GMIP, HAP1, HAPLN1, HBXIP, HCAR1, HDAC6, HGS, HIST1H3A, HIST1H3B, HIST1H3C, HIST1H3D, HIST1H3E, HIST1H3F, HIST1H3G, HIST1H3H, HIST1H3I, 20) HIST1H3J, HK2, HMGB1, HPR, HSF2BP, HSP90AA1, HSPA8, IFI16, IPPK, IRGM, IST1, ITGB4, ITPKC, KCNK3, KCNQ1, KIAA0226, KIAA1324, KRCC1, KRT15, KRT73, LAMP1, LAMP2, LAMTOR1, LAMTOR2, LAMTOR3, LARP1B, LENG9, LGALS8, LIX1, LIX1L, LMCD1, LRRK2, LRSAM1, LSM4, MAP1A, MAP1LC3A, MAP1LC3B, MAP1LC3B2, MAP1LC3C, MAPIS, MAP2K1, MAP3K12, MARK2, MBD5, MDH1, MEX3C, MFN1, MFN2, MLST8, MRPS10, MRPS2, MSTN, MTERFD1, MTMR14, MTMR3, MTOR, MTSS1, MYH11, MYLK, MYOM1, NBR1, NDUFB9, NEFM, NHLRC1, NME2, NPC1, NR2C2, NRBF2, NTHL1, NUP93, OBSCN, OPTN, P2RX5, PACS2, PARK2, PARK7, PDK1, PDK4, PEX13, PEX3, PFKP, PGK2, PHF23, PHYHIP, PI4K2A, PIK3C3, PIK3CA, PIK3CB, PIK3R4, PINK1, PLEKHM1, PLOD2, PNPO, PPARGC1A, PPY, PRKAA1, PRKAA2, PRKAB1, PRKAB2, PRKAG1, PRKAG2, PRKAG3, PRKD2, PRKG1, PSEN1, PTPN22, RAB12, RAB1A, RAB1B, RAB23, RAB24, RAB33B, RAB39, RAB7A, RB1CC1, RBM18, REEP2, REP15, RFWD3, RGS19, RHEB, RIMS3, RNF185, RNF41, RPS27A, RPTOR, RRAGA, RRAGB, RRAGC, RRAGD, S100A8, S100A9, SCN1A, SERPINB10, SESN2, SFRP4, SH3GLB1, SIRT2, SLC1A3, SLC1A4, SLC22A3, SLC25A19, SLC35B3, SLC35C1, SLC37A4, SLC6A1, SLCO1A2, SMURF1, SNAP29, SNAPIN, SNF8, SNRPB, SNRPB2, SNRPD1, SNRPF, SNTG1, SNX14, SPATA18, SQSTM1, SRPX, STAM, STAM2, STAT2, STBD1, STK11, STK32A, STOM, STX12, STX17, SUPT3H, TBC1D17, TBC1D25, TBC1D5, TCIRG1, TEAD4, TECPR1, TECPR2, TFEB, TM9SF1, TMBIM6, TMEM203, TMEM208, TMEM39A, TMEM39B, TMEM59, TMEM74, TMEM93, TNIK, TOLLIP, TOMM20, TOMM22, TOMM40, TOMM5, TOMM6, TOMM7, TOMM70A, TP53INP1, TP53INP2, TRAPPC8, TREM1, TRIM17, TRIM5, TSG101, TXLNA, UBA52, UBB, UBC, UBQLN1, UBQLN2, UBQLN4, ULK1, ULK2, ULK3, USP10, USP13, USP30, UVRAG, VAMP7, VAMP8, VDAC1, VMP1, VPS11, VPS16, VPS18, VPS25, VPS28, VPS33A, VPS33B, VPS36, VPS37A, VPS37B, VPS37C, VPS37D, VPS39, VPS41, VPS4A, VPS4B, VTA1, VTI1A, VTI1B, WDFY3, WDR45, WDR45L, WIPI1, WIPI2, XBP1, YIPF1, ZCCHC17, ZFYVE1, ZKSCAN3, ZNF189, ZNF593, and ZNF681.

The gene set “Up resting vs. Down activated” includes the following genes: ABCA7, ABCF3, ACAP2, AMT, ANKH, ATF7IP2, ATG14, ATP1A1, ATXN7, ATXN7L3B, BCL7A, BEX4, BSDC1, BTG1, BTG2, BTN3A1, C11orf21, C19orf22, C21orf2, CAMK2G, CARS2, CCNL2, CD248, CD5, CD55, CEP164, CHKB, CLK1, CLK4, CTSL1, DBP, DCUN1D2, DENND1C, DGKD, DLG1, DUSP1, EAPP, ECE1, ECHDC2, ERBB2IP, FAM117A, FAM134B, FAM134C, FAM169A, FAM190B, FAU, FLJ10038, FOXJ2, FOXJ3, FOXL1, FOXO1, FXYD5, FYB, HLA-E, HSPA1L, HYAL2, ICAM2, IFIT5, IFITM1, IKBKB, IQSEC1, IRS4, KIAA0664L3, KIAA0748, KLF3, KLF9, KRT18, LEF1, LINC00342, LIPA, LIPT1, LLGL2, LMBR1L, LPAR2, LTBP3, LYPD3, LZTFL1, MANBA, MAP2K6, MAP3K1, MARCH8, MAU2, MGEA5, MMP8, MPO, MSL1, MSL3, MYH3, MYLIP, NAGPA, NDST2, NISCH, NKTR, NLRP1, NOSIP, NPIP, NUMA1, PAIP2B, PAPD7, PBXIP1, PCIF1, PI4KA, PLCL2, PLEKHA1, PLEKHF2, PNISR, PPFIBP2, PRKCA, PRKCZ, PRKD3, PRMT2, PTP4A3, PXN, RASA2, RASA3, RASGRP2, RBM38, REPIN1, RNF38, RNF44, ROR1, RPL30, RPL32, RPLP1, RPS20, RPS24, RPS27, RPS6, RPS9, RXRA, RYK, SCAND2, SEMA4C, SETD1B, SETD6, SETX, SF3B1, SH2B1, SLC2A4RG, SLC35E2B, SLC46A3, SMAGP, SMARCE1, SMPD1, SNPH, SP140L, SPATA6, SPG7, SREK1IP1, SRSF5, STAT5B, SVIL, SYF2, SYNJ2BP, TAF1C, TBC1D4, TCF20, TECTA, TES, TMEM127, TMEM159, TMEM30B, TMEM66, TMEM8B, TP53TG1, TPCN1, TRIM22, TRIM44, TSC1, TSC22D1, TSC22D3, TSPYL2, TTC9, TTN, UBE2G2, USP33, USP34, VAMP1, VILL, VIPR1, VPS13C, ZBED5, ZBTB25, ZBTB40, ZC3H3, ZFP161, ZFP36L1, ZFP36L2, ZHX2, ZMYM5, ZNF136, ZNF148, ZNF318, ZNF350, ZNF512B, ZNF609, ZNF652, ZNF83, ZNF862, and ZNF91.

The gene set “Progressively up in memory differentiation” includes the following genes: MTCH2, RAB6C, KIAA0195, SETD2, C2orf24, NRD1, GNA13, COPA, SELT, TNIP1, CBFA2T2, LRP10, PRKC1, BRE, ANKS1A, PNPLA6, ARL6IP1, WDFY1, MAPK1, GPR153, SHKBP1, MAP1LC3B2, PIP4K2A, HCN3, GTPBP1, TLN1, C4orf34, KIF3B, TCIRG1, PPP3CA, ATG4D, TYMP, TRAF6, C17orf76, WIPF1, FAM108A1, MYL6, NRM, SPCS2, GGT3P, GALK1, CLIP4, ARL4C, YWHAQ, LPCAT4, ATG2A, IDS, TBC1D5, DMPK, ST6GALNAC6, REEP5, ABHD6, KIAA0247, EMB, TSEN54, SPIRE2, PIWIL4, ZSCAN22, ICAM1, CHD9, LPIN2, SETD8, ZC3H12A, ULBP3, IL15RA, HLA-DQB2, LCP1, CHP, RUNX3, TMEM43, REEP4, MEF2D, ABL1, TMEM39A, PCBP4, PLCD1, CHST12, RASGRP1, C1orf58, C11orf63, C6orf129, FHOD1, DKFZp434F142, PIK3CG, ITPR3, BTG3, C4orf50, CNNM3, IFI16, AK1, CDK2AP1, REL, BCL2L1, MVD, TTC39C, PLEKHA2, FKBP11, EML4, FANCA, CDCA4, FUCA2, MFSD10, TBCD, CAPN2, IQGAP1, CHST11, PIK3R1, MYO5A, KIR2DL3, DLG3, MXD4, RALGDS, SIPR5, WSB2, CCR3, TIPARP, SP140, CD151, SOX13, KRTAP5-2, NF1, PEA15, PARP8, RNF166, UEVLD, LIMK1, CACNB1, TMX4, SLC6A6, LBA1, SV2A, LLGL2, IRF1, PPP2R5C, CD99, RAPGEF1, PPP4R1, OSBPL7, FOXP4, SLA2, TBC1D2B, ST7, JAZF1, GGA2, PI4K2A, CD68, LPGAT1, STX11, ZAK, FAM160B1, RORA, C8orf80, APOBEC3F, TGFBI, DNAJC1, GPR114, LRP8, CD69, CMIP, NAT13, TGFB1, FLJ00049, ANTXR2, NR4A3, IL12RB1, NTNG2, RDX, MLLT4, GPRIN3, ADCY9, CD300A, SCD5, ABI3, PTPN22, LGALS1, SYTL3, BMPR1A, TBK1, PMAIP1, RASGEF1A, GCNT1, GABARAPL1, STOM, CALHM2, ABCA2, PPP1R16B, SYNE2, PAM, C12orf75, CLCF1, MXRA7, APOBEC3C, CLSTN3, ACOT9, HIP1, LAG3, TNFAIP3, DCBLD1, KLF6, CACNB3, RNF19A, RAB27A, FADS3, DLG5, APOBEC3D, TNFRSF1B, ACTN4, TBKBP1, ATXN1, ARAP2, ARHGEF12, FAM53B, MAN1A1, FAM38A, PLXNC1, GRLF1, SRGN, HLA-DRB5, B4GALT5, WIPI1, PTPRJ, SLFN11, DUSP2, ANXA5, AHNAK, NEO1, CLIC1, EIF2C4, MAP3K5, IL2RB, PLEKHG1, MYO6, GTDC1, EDARADD, GALM, TARP, ADAM8, MSC, HNRPLL, SYT11, ATP2B4, NHSL2, MATK, ARHGAP18, SLFN12L, SPATS2L, RAB27B, PIK3R3, TP53INP1, MBOAT1, GYG1, KATNAL1, FAM46C, ZC3HAV1L, ANXA2P2, CTNNA1, NPC1, C3AR1, CRIM1, SH2D2A, ERN1, YPEL1, TBX21, SLC1A4, FASLG, PHACTR2, GALNT3, ADRB2, PIK3AP1, TLR3, PLEKHA5, DUSP10, GNAO1, PTGDR, FRMD4B, ANXA2, EOMES, CADM1, MAF, TPRG1, NBEAL2, PPP2R2B, PELO, SLC4A4, KLRF1, FOSL2, RGS2, TGFBR3, PRF1, MYO1F, GAB3, C17orf66, MICAL2, CYTH3, TOX, HLA-DRA, SYNE1, WEE1, PYHIN1, F2R, PLD1, THBS1, CD58, FAS, NETO2, CXCR6, ST6GALNAC2, DUSP4, AUTS2, C1orf21, KLRG1, TNIP3, GZMA, PRR5L, PRDM1, ST8SIA6, PLXND1, PTPRM, GFPT2, MYBL1, SLAMF7, FLJ16686, GNLY, ZEB2, CST7, IL18RAP, CCL5, KLRD1, and KLRB1.

The gene set “Up TEM vs. Down TN” includes the following genes: MYO5A, MXD4, STK3, SIPR5, GLCCI1, CCR3, SOX13, KRTAP5-2, PEA15, PARP8, RNF166, UEVLD, LIMK1, SLC6A6, SV2A, KPNA2, OSBPL7, ST7, GGA2, PI4K2A, CD68, ZAK, RORA, TGFBI, DNAJC1, JOSD1, ZFYVE28, LRP8, OSBPL3, CMIP, NAT13, TGFB1, ANTXR2, NR4A3, RDX, ADCY9, CHN1, CD300A, SCD5, PTPN22, LGALS1, RASGEF1A, GCNT1, GLUL, ABCA2, CLDND1, PAM, CLCF1, MXRA7, CLSTN3, ACOT9, METRNL, BMPR1A, LRIG1, APOBEC3G, CACNB3, RNF19A, RAB27A, FADS3, ACTN4, TBKBP1, FAM53B, MAN1A1, FAM38A, GRLF1, B4GALT5, WIPI1, DUSP2, ANXA5, AHNAK, CLIC1, MAP3K5, ST8SIA1, TARP, ADAM8, MATK, SLFN12L, PIK3R3, FAM46C, ANXA2P2, CTNNA1, NPC1, SH2D2A, ERN1, YPEL1, TBX21, STOM, PHACTR2, GBP5, ADRB2, PIK3AP1, DUSP10, PTGDR, EOMES, MAF, TPRG1, NBEAL2, NCAPH, SLC4A4, FOSL2, RGS2, TGFBR3, MYO1F, C17orf66, CYTH3, WEE1, PYHIN1, F2R, THBS1, CD58, AUTS2, FAM129A, TNIP3, GZMA, PRR5L, PRDM1, PLXND1, PTPRM, GFPT2, MYBL1, SLAMF7, ZEB2, CST7, CCL5, GZMK, and KLRB1.

Other gene sets describing similar processes and/or characteristics can also be used to characterize cell phenotypes described above.

Cell Ranger VDJ was used to generate single cell VDJ sequences and annotations for each single cell 5′ library. Loupe Cell Browser software and Bioconductor packages were used for data analysis and visualization.

### Results

This example aims to compare T cell states between purified T cells which served as input cells, CART cells manufactured using the ARM process (labeled as “Day 1” cells), and CART cells manufactured using the TM process (labeled as “Day 9” cells) using single-cell RNA-seq (scRNA-seq). In addition, single-cell TCR-seq (scTCR-seq) was performed to study clonality and track cell differentiation from input to post-manufacturing materials.

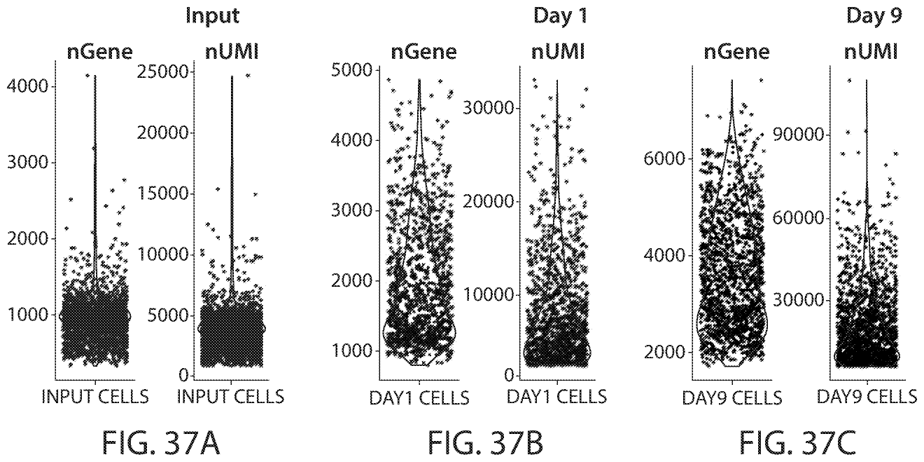

As shown in FIGS. 37A-37C, input cells had the fewest expressed genes and UMIs, suggesting these cells were not transcriptionally active and were in a resting state. Day 1 and Day 9 cells were expressing more genes, with Day 9 cells being the most transcriptionally active. Similar results are shown in FIGS. 38A-38D. Input cells were not expressing proliferation genes (FIGS. 38A and 38D).

Additional gene set analysis data are shown in FIGS. 39A-39E. Different populations of cells were compared using the median gene set scores. Day 1 cells and input cells were in a younger, more stem-like memory state (FIGS. 39A-39C). In FIG. 39A, the median GeneSetScore (Up TEM vs. Down TSCM) values for Day 1 cells, Day 9 cells, and input cells are −0.084, 0.035, and −0.1, respectively. In FIG. 39B, the median GeneSetScore (Up Treg vs. Down Teff) values for Day 1 cells, Day 9 cells, and input cells are −0.082, 0.087, and −0.071, respectively. In FIG. 39C, the median GeneSetScore (Down stemness) values for Day 1 cells, Day 9 cells, and input cells are −0.062, 0.14, and −0.081, respectively.

In addition, Day 1 cells were in a more ideal metabolic state compared to Day 9 cells (FIGS. 39D and 39E). In FIG. 39D, the median GeneSetScore (Up hypoxia) values for Day 1 cells, Day 9 cells, and input cells are 0.019, 0.11, and −0.096, respectively. In FIG. 39E, the median GeneSetScore (Up autophagy) values for Day 1 cells, Day 9 cells, and input cells are 0.066, 0.11, and −0.09, respectively.

Based on gene expression, the input cells contain four clusters. Cluster 0 is characterized by high expression of LMNA, S100A4, etc. Cluster 1 is characterized by high expression of RP913, PRKCQ-AS1, etc. Cluster 2 is characterized by high expression of PR11-291B21.2, CD8B, etc. Cluster 3 is characterized by high expression of NKG7, GZMH, CCL5, CST7, GNLY, FGFBP2, GZMA, CCL4, CTSW, CD8A, etc. In a T-Distributed Stochastic Neighbor Embedding (TSNE) plot for the input cells, Cluster 3 stood out from the other cells, and Cluster 1 and Cluster 2 were hard to differentiate.

According to the gene set analysis shown in FIGS. 40A-40C, Cluster 0 and Cluster 3 were enriched for a T regulatory phenotype compared to Cluster 1 and Cluster 2 which were enriched for a T effector phenotype. Cluster 3 was dominated by late memory/effector memory (TEM) cells, Cluster 1 and Cluster 2 were early memory and naïve cells, and Cluster 0 is in the middle. The majority of the input cells were in an early memory, naïve state. Without wishing to be bound by theory, these cells may do the best during the manufacturing procedure.

Less transcriptional heterogeneity was seen in Day 1 cells and Day 9 cells (data not shown).

Like the input population, Day 1 cells showed a large cluster of early memory cells and a smaller cluster of late memory cells in a TSNE plot. similar to what was seen with Cluster 3 of the input cells. In contrast, Day 9 cells did not show distinct clusters of early memory cells in a TSNE plot. This implies that by day 9, the cells had become more homogeneous.

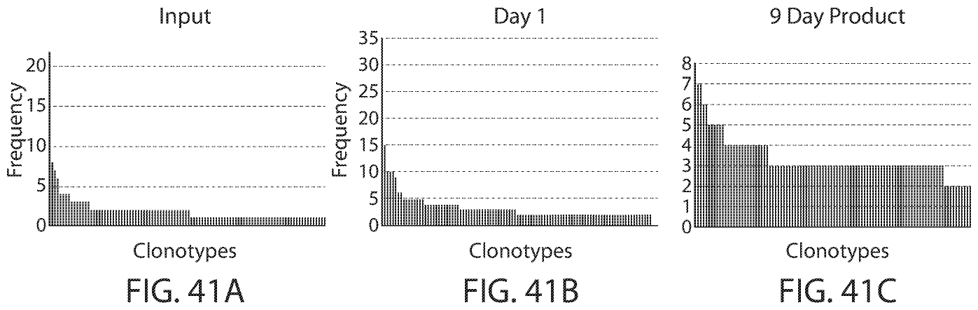

TCRs were sequenced and clonotype diversity was measured. Overall, the three clonotype profiles were very flat-most clones were only picked up once (FIGS. 41A-41C and Table 24). Shannon entropy in Table 24 measures the flatness of the distribution. The dominant clones in the input cells were late memory cells. Day 1 cells looked similar to the input cells but started to even out. By day 9, the dominate clones had substantially evened out and the distribution was much more flat. The diversity measurement was the highest at day 9 because there was a much more even and flat distribution in Day 9 cells than in the input cells or Day 1 cells.

TABLE 24
Measurements of TCR diversity

| | Input | Day 1 product | Day 9 product |
|---|---|---|---|
| Average clones per | 1.10 | 1.05 | 1.07 |
| clonotype | | | |
| Estimated number of | 7344 | 7687 | 7233 |
| cells | | | |
| Total number of | 5325 | 7403 | 6736 |
| clonotypes | | | |
| Diversity | 342.27 | 802.94 | 3382.62 |
| Normalized Shannon | 9.98E−01 | 9.95E−01 | 9.96E−01 |
| entropy | | | |

### Summary

There were significant T cell state differences between Day 1 and Day 9 products. Day 1 cells were much more similar to input cells and had enrichment for stemness signatures, indicating a more efficacious product.

### Example 11: Phase I, Open Label, Study of B-Cell Maturation Antigen (BCMA)-Directed CAR-T Cells in Adult Patients with Relapsed and/or Refractory Multiple Myeloma (MM)

This study evaluates the safety and tolerability of anti-BCMA CART-T cell therapy in adult MM subjects who are relapsed and/or refractory to at least two prior treatment regimens, including an IMiD (e.g. lenalidomide or pomalidomide), a proteasome inhibitor (e.g. bortezomib, carfilzomib), and an approved anti-CD38 antibody (e.g. daratumumab), if available, and have documented evidence of disease progression (IMWG criteria).

The anti-BCMA CAR comprises a PI61 anti-BCMA scFv, a CD8 hinge and transmembrane region, a 4-1BB costimulatory domain, and a CD3 zeta signaling domain. In this study, the anti-BCMA CAR-T cell products are manufactured using the Activated Rapid Manufacturing (ARM) process. Such cells are referred to as “ARM-BCMA CAR.” Specifically, T cells are enriched from a subject's leukapheresis unit using commercially available magnetic beads capturing CD4 and CD8 co-receptors on the T cell surface. Enriched T cells are then stimulated with a colloidal polymeric nanomatrix covalently attached to humanized recombinant agonist antibodies against human CD3 and CD28. Twenty-four hours after seeding, activation and transduction, CAR-T cells are harvested and washed to remove residual non-integrated vector and non-bound activating matrix. After the wash, BCMA CART cell therapy is concentrated and cryopreserved. Results from a release testing procedure are required prior to release of the product for administration.

Compared to the TM process for CAR-T cells, which relies on an ex vivo T-cell expansion period lasting 7-8 days after transduction with lentiviral vector, the ARM process does not include ex vivo T-cell expansion. In contrast, ARM produced T cells are harvested 24 hours after gene transfer, allowing them to expand in vivo in patients. The greater in vivo T cell expansion achieved with the ARM process is predicted to result in a less differentiated T cell phenotype, preserving a greater fraction of memory stem T cells in the final cell product. The presence of less differentiated, memory CAR-T cells has been associated with improved antitumor efficacy in clinical studies (Fraietta J A, et al., (2018) Nat Med, 24 (5); 563-71). Without wishing to be bound by theory, BCMA CART cells comprised of a greater fraction of memory T stem cells result in enhanced CAR-T cell expansion in patients, thus overcoming effector T cell exhaustion and resulting in more durable efficacy in MM patients compared with BCMA CARTs produced under traditional manufacturing processes.

The ARM process produces CAR-T cells composed of a significantly greater proportion of naïve-like memory T cells (CCR7+/CD45RO−) in both the overall product and the CAR-positive fraction as compared to CART cells manufactured using the traditional manufacturing (TM) process. ARM-BCMA CAR has shown tumor eradication in preclinical MM models in a dose responsive fashion. ARM-BCMA CAR is at least five-fold more potent as compared to BCMA CAR-T cells generated with the TM process and led to extended CAR-T expansion in vivo, with higher levels of systemic cytokines. Together, these results support the hypothesis that anti-BCMA CAR-T cell products manufactured with the ARM process contain T cells with a pronounced memory stem cell phenotype, resulting in a BCMA CAR-T cell product with enhanced engraftment, expansion, and anti-MM properties.

In this phase I study, each subject is first evaluated for clinical eligibility during screening. Subjects eligible for inclusion in this study must meet all of the following criteria: (1) ≥18 years of age at the time of ICF signature; (2) ECOG performance status that is either 0 or 1 at screening; (3) subjects with MM who are relapsed and/or refractory to at least 2 prior treatment regimens, including an IMiD (e.g. lenalidomide or pomalidomide), a proteasome inhibitor (e.g. bortezomib, carfilzomib), and an approved anti-CD38 antibody (e.g. 20) daratumumab) (if available) and have documented evidence of disease progression (IMWG criteria); (4) subjects must have measurable disease defined by at least 1 of the following 3 measurements: serum M-protein≥1.0 g/dL, urine M-protein≥200 mg/24 hours, or serum free light chain (sFLC)>100 mg/L of involved FLC; (5) All patients must be suitable for serial bone marrow biopsy and/or aspirate collection according to institution's guidelines and be willing to undergo this repeated procedure as described for this study; (6) subjects must meet the following hematological values at screening: absolute neutrophil count (ANC)≥1,000/mm³ (≥1×10⁹/L) without growth factor support within 7 days prior to testing, absolute number of CD3+ T cells>150/mm³ (>0.15×10⁹/L) without transfusion support within 7 days prior to testing, platelets≥50 000/mm³ (≥50×10⁹/L), and hemoglobin≥8.0 g/dl (≥4.9 mmol/L); (7) patient must be deemed suitable by investigator to undergo fludarabine/cyclophosphamide LD regimen; and (8) must have a leukapheresis material of non-mobilized cells accepted for manufacturing. If eligible, a subject has a leukapheresis product collected and submitted for CAR-T manufacture. The subject is enrolled with the acceptance of their leukapheresis product for the start of manufacture.

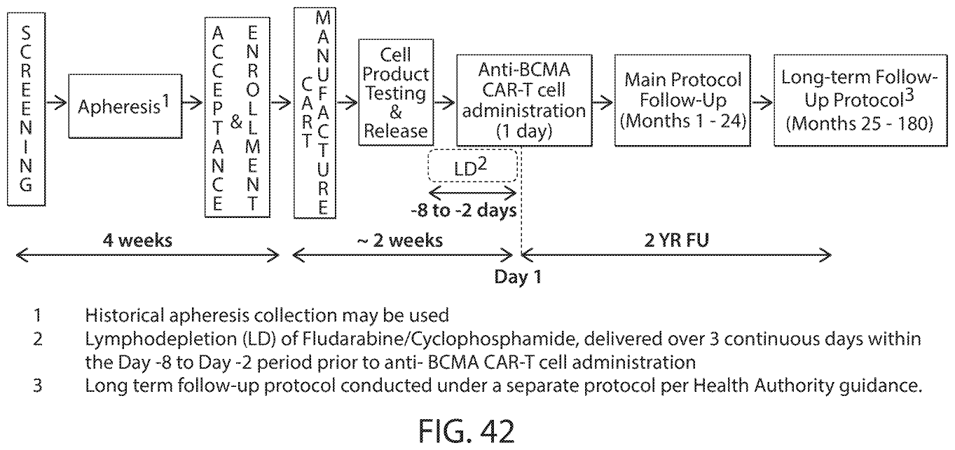

Subjects receive lymphodepletion (LD) chemotherapy only after the final product has been confirmed to be available. Following LD chemotherapy, a single dose of anti-BCMA CAR-T cell product is administered via an intravenous (i.v.) injection to a subject within 90 minutes from thawing (FIG. 42). The starting dose of ARM-BCMA CAR is 1×10⁷ viable CAR-positive T cells. The dose of 5×10⁷ viable CAR-positive T cells is also tested. Each subject is hospitalized for the first 72 hours following anti-BCMA CAR-T cell administration.

For pharmacokinetic analysis, serial blood samples are collected at different time points to measure ARM-BCMA CAR cellular kinetics in peripheral blood by flow cytometry and qPCR, in bone marrow by flow cytometry and qPCR, to measure cellular and humoral immunogenicity, and to measure potential pharmacodynamic markers including sBCMA, BAFF, and APRIL, in peripheral blood by ELISA. In particular, subjects are analyzed for the amount of CAR transgene in peripheral blood, bone marrow, or other relevant tissues; the surface expression of CAR-positive T cells in the peripheral blood or bone marrow; the anti-mCAR antibodies in the serum; the percentage of IFN-γ positive CD4/CD8 T cells in PBMC; markers of immune cell activation; soluble immune factors and cytokines (e.g., sBCMA, IFN-γ, IL-2, IL-4, IL-6, IL-8, IL-10, IL-15, TNF-α), CAR-T clonality; and the levels of soluble BCMA, APRIL, and BAFF in the serum.

### Example 12: Manufacturing BCMA CART Cells Using the Activated Rapid Manufacturing (ARM) Process

The ARM process of BCMA CART cells initiates with the preparation of the media as outlined in Table 25.

Cryopreserved leukapheresis product is used as the starting material and is processed for T cell enrichment. When available, the apheresis paper work is utilized to define the T cell percentage. In the absence of the T cell percentage data on the apheresis paperwork, the sentinel vial testing is performed on incoming cryopreserved leukapheresis products to obtain T cell percentage target for the apheresis. The results for the T cell percentage determine how many bags are thawed on Day 0 of the ARM process.

TABLE 25
Media and Buffer type and point of use during
BCMA CART manufacturing

| Media Type | Source | Point of Use |
|---|---|---|
| CliniMACS ® Buffer/ | Prepared by | Day 0 Processing on |
| human serum albumin | operator on day 0 | Cell Wash/Separator |
| (HSA) (0.5% in | | |
| working concentration) | | |
| Rapid Media | Prepared by | Day 0 for Cell |
| | operator on day 0 | Seeding |
| PBS/ HSA (1% or 2% in | Prepared by | Harvest and culture |
| working concentration) | operator on day 0 | Wash Media (Day 1) |
| Cryostor10 (CS10) | Commercially | Harvest Formulation |
| | available | |

Cryopreserved leukapheresis is thawed, washed, and then undergoes T cell selection and enrichment using CliniMACS® microbead technology. Viable nucleated cells (VNCs) are activated with TransACT (Miltenyi) and transduced with a lentiviral vector encoding the CAR. The viable cells selected with the Miltenyi microbeads are seeded into the centricult on the Prodigy®, which is a non-humidified incubation chamber. While in culture, the cells are suspended in Rapid media, which is an OpTmizer™ CTS™ based medium that contains the CTS™ Supplement (ThermoFisher), Glutamax, IL-2 and 2% Immune cell serum replacement amongst its components to promote T cell activation and transduction. Lentiviral transduction is performed once on the day of seeding after the TransACT has been added to the diluted cells in the culture media. Lentiviral vector will be thawed immediately prior to use on day of seeding for up to 30 minutes at room temperature.

From the start of the process on Day 0 to the initiation of the culture wash and harvest, BCMA CART cells are cultured for 20-28 hours from seeding. Following culture, the cell suspension undergoes two culture washes and one harvest wash within the centricult chamber (Miltenyi Biotech).

After the harvest wash on the CliniMACS® Prodigy® on day 1, the cell suspension is sampled to determine viable cell count and viability. Cell suspension is then transferred to a centrifuge to be pelleted manually. The supernatant is removed, and the cell pellet is re-suspended in CS10 (BioLife Solution), resulting in a product formulation with a final DMSO concentration of ˜10.0%. The viable cell count is formulated at the end of harvest for dosing. The doses are then distributed into individual cryobags and analytical sampling into cryovials.

Cryopreserved products are stored in monitored LN2 storage tanks, in a secure, limited access area until final release and shipping.

### Example 13: Characterization of BCMA CART Cells Manufactured Using the Activated Rapid Manufacturing (ARM) Process

### Summary

This example describes characterization of BCMA CART cells manufactured using the ARM process. The ARM process produces CAR-T cells composed of a significantly greater proportion of naïve-like memory T cells (CCR7+/CD45RO−) as compared to the traditional manufacturing (TM) product. In a preclinical model of multiple myeloma (MM), BCMA CART cells manufactured using the ARM process induced tumor regression in a dose-dependent manner and was up to 5-fold more efficacious in killing tumors compared to BCMA CART cells manufactured using the TM process. In addition, ARM-manufactured cells showed extended CART expansion in vivo (up to 3 folds higher Cmax and AUC0-21d) and induced higher systemic cytokines (IFN-γ by 3.5 folds) compared to TM-manufactured cells. Together, these results support the hypothesis that BCMA CART cells manufactured with the ARM process contain T cells with a pronounced memory stem cell phenotype and an enhanced in vivo expansion potential.

Using the ARM process, CAR could be stably expressed at 96 h after viral addition (also referred to as 72 h at post-thaw of the product). Therefore, 96 h post-viral addition or 72 h post-thaw is considered to be a surrogate time point for CAR expression for in vitro and in vivo activity. BCMA CART cells manufactured using the ARM process preserve a less differentiated cell population, and show higher target specific cytokine production in vitro, when compared to BCMA CART cells manufactured under the TM process.

BCMA CART cells manufactured using the ARM process demonstrated high specificity to BCMA using a commercial human plasma membrane protein array. The assay detected binding to BCMA (TNFRSF17) but no other strong, medium, or weak binders. The screen did not identify with any high confidence the presence of cross-reacting proteins of the anti-human BCMA single chain antibody variable fragment (scFv) (PI61) expressed in the BCMA CART product. Target distribution studies were performed to determine potential off-tumor on-target toxicity. Immunohistochemistry (IHC), in situ hybridization (ISH), and polymerase chain reaction (PCR) assays were utilized to examine the distribution of BCMA in normal human tissues. These analyses demonstrated that BCMA expression was limited to sites containing normal plasma cells (PCs), such as secondary lymphoid organs, bone marrow and mucosal associated lymphoid tissues. Because central nervous system (CNS) neurotoxicity has been a concern with other cell-based therapies, expression in brain was examined. No staining in the CNS was observed by immunohistochemistry using a commercially available antibody shown to be specific for BCMA nor by binding assays using a human-rabbit chimeric tool antibody containing a BCMA targeting scFv. These findings were confirmed by the absence of BCMA mRNA in these tissues as measured by in situ hybridization and PCR based splice variant analysis. BCMA CART targeting of normal PCs and BCMA-expressing plasmacytoid dendritic cells is likely to result in their depletion; however, targeting of other cell types is not anticipated.

### Results

The studies described below compared BCMA CART cells manufactured using the ARM process (referred to as “ARM-BCMA CAR”) with BCMA CART cells manufactured using the TM process (referred to as “TM-BCMA CAR” or “TM-BCMA CAR*”). The CAR expressed in ARM-BCMA CAR and the CAR expressed in TM-BCMA CAR* have the same sequence, comprising a PI61 scFv, a CD8 hinge and transmembrane region, a 4-1BB costimulatory domain, and a CD3 zeta signaling domain. The CAR expressed in TM-BCMA CAR comprises a BCMA10 scFv, a CD8 hinge and transmembrane region, a 4-1BB costimulatory domain, and a CD3 zeta signaling domain.

### ARM-BCMA CAR Expression Kinetics In Vitro

In contrast to TM which measures lentiviral integration of the CAR transgene after 8-9 days, in the ARM process, the lentiviral transgene may not be fully integrated and truly expressed within 24 h post lentiviral addition, as lentiviral pseudotransduction could occur (Haas D L, et al., (2000) Mol Ther; 2 (1): 71-80; Galla M, et al., (2004) Mol Cell; 16 (2): 309-15). Therefore, the BCMA-CAR expression pattern was evaluated over time by extended culturing of ARM-BCMA CAR in vitro in the presence or absence of 3′-azido-3′-deoxythymidine (AZT) to evaluate the potential pseudotransduction versus stable integration and expression of the CAR transgene. Flow cytometry (FACS) analyses were performed to detect CAR surface expression at 24 h, 48 h, 72 h, 96 h and 168 h post T cell activation and transduction with the lentiviral vector. In some cases, ARM-BCMA CAR and an aliquot of this product were frozen down immediately upon harvest for additional characterization in other assays.

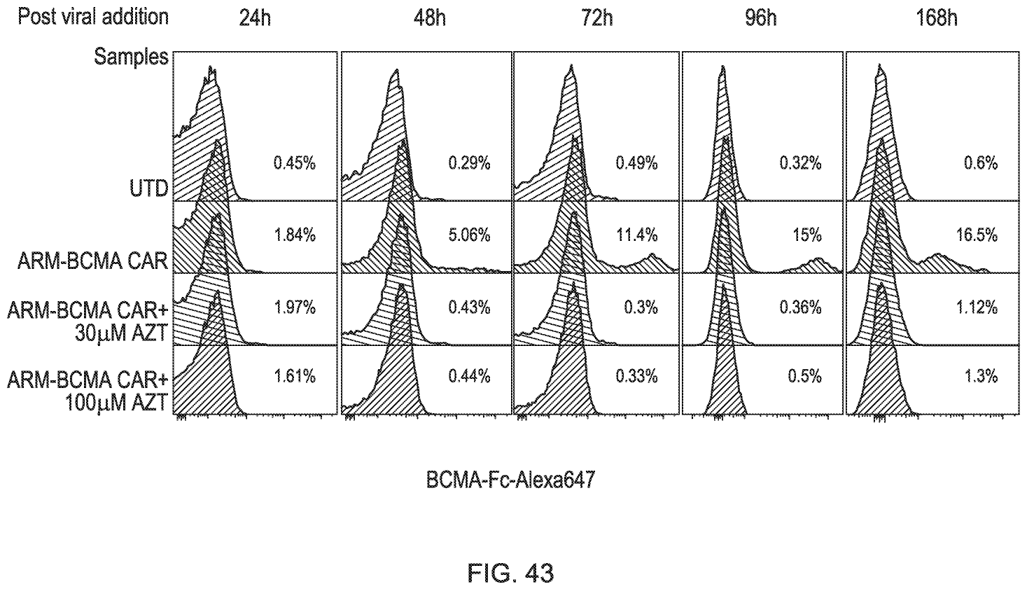

As shown in FIG. 43, FACS analyses indicate that the BCMA-CAR revealed practically no expression at 24 h after the addition of the lentiviral vector. However, the CAR+ population initially emerged at 48 h. The CAR+ population slightly increased at each time point from 48 h to 168 h after viral addition. CAR seemed to be stably expressed starting from 96 h. This contrasts with the untransduced (UTD) and AZT treated samples, which showed no CAR+ population at any time point from 48 h (FIG. 43). AZT was able to effectively inhibit CAR expression at both the 30 μM and 100 μM doses, suggesting that BCMA-CAR expression is due to viral gene integration into the host cell genome and unlikely a consequence of lentiviral pseudo-transduction.

### ARM-BCMA CAR Preserves T Cell Stemness

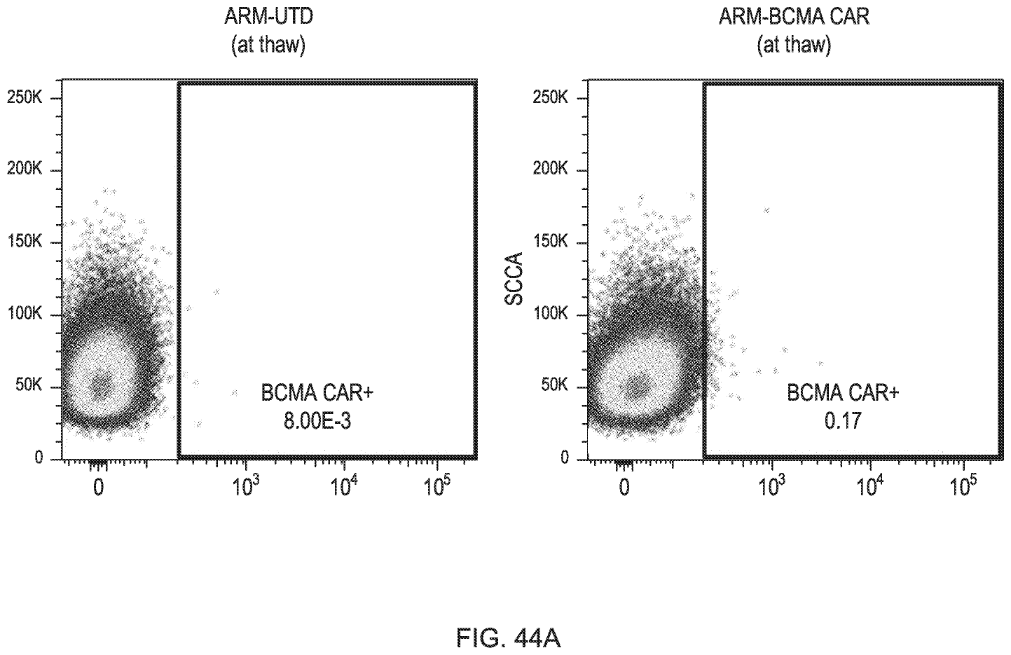

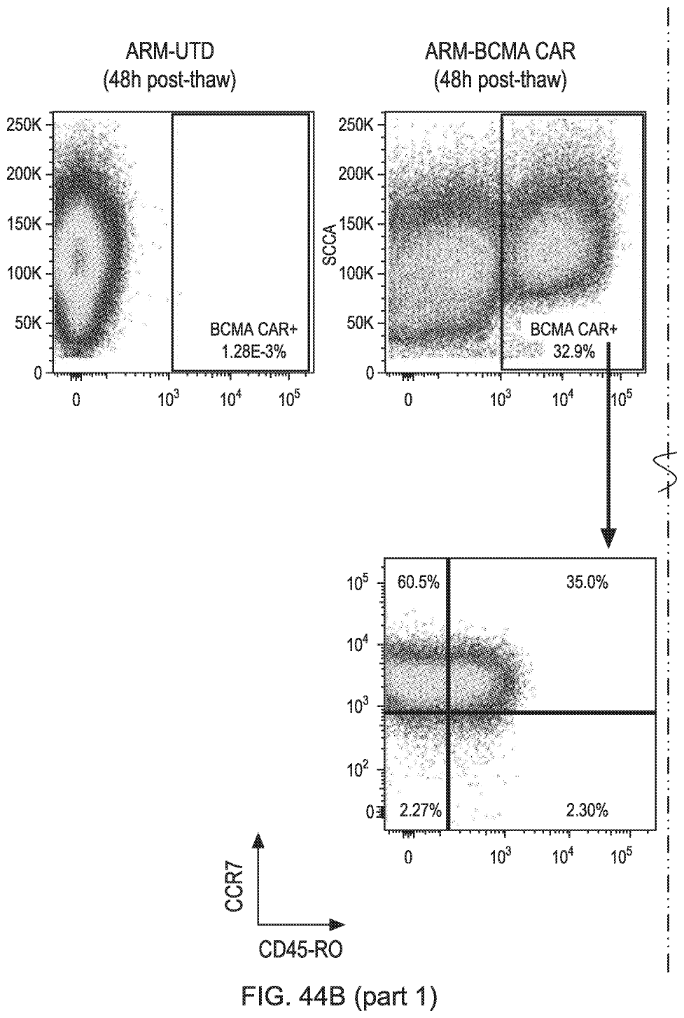

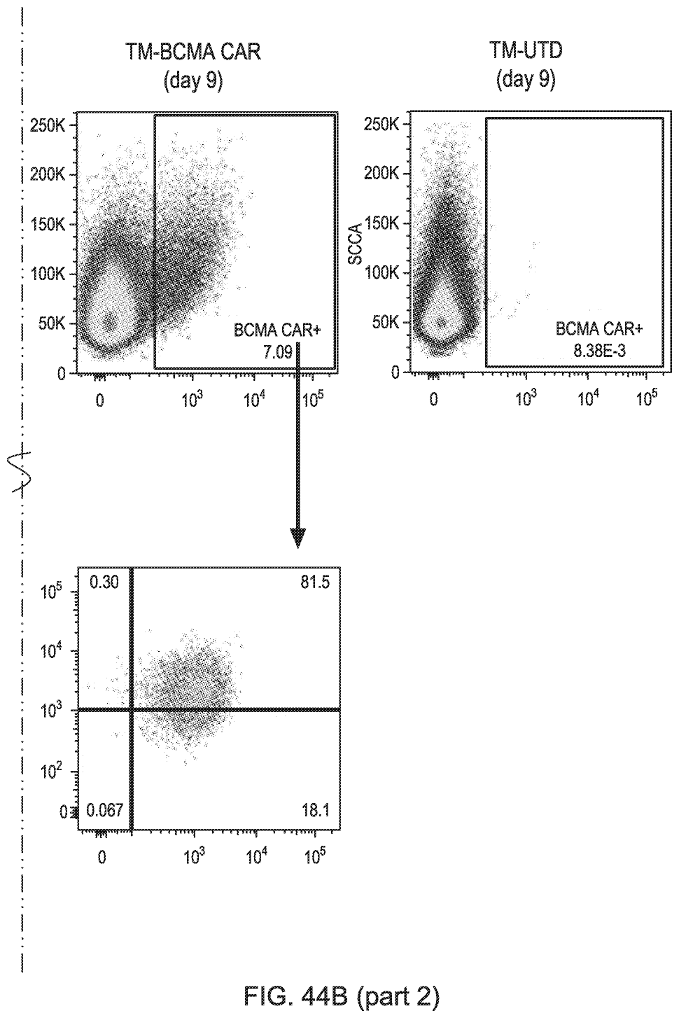

ARM-BCMA CAR and TM-BCMA CAR were analyzed by FACS to evaluate CAR expression at thaw, as well as the T-cell phenotype at 48 h post thaw (FIGS. 44A and 44B). BCMA-CAR was almost undetectable at thaw seen in two donors (FIG. 44A), which is consistent with the observation in the CAR expression kinetics study shown in FIG. 43. However, at 48 h post-thaw, BCMA-CAR expression was 32.9% for ARM-BCMA CAR. In contrast, TM-BCMA CAR revealed BCMA-CAR expression of 7% (FIG. 44B). Analysis of the CAR+ T-cell phenotype revealed that the ARM process retained naïve-like T cells (60% CD45RO−/CCR7+), which proved to be 26 folds more than the effector memory T cell population (CD45RO+/CCR7−). The TM process mainly resulted in central-memory T cells (81% CD45RO+/CCR7+) within CAR+ T cells. The naive-like T cell population was nearly absent with the TM process. This naive T-cell population largely overlaps with CD45RO−/CD27+ Tstem cells (described by Cohen A D, et al., (2019) J Clin Invest; 129 (6): 2210-21; and Fraietta, et al (2018) Nat Med, 24 (5); 563-571) and is associated with enhanced CAR-T expansion and clinical responses.

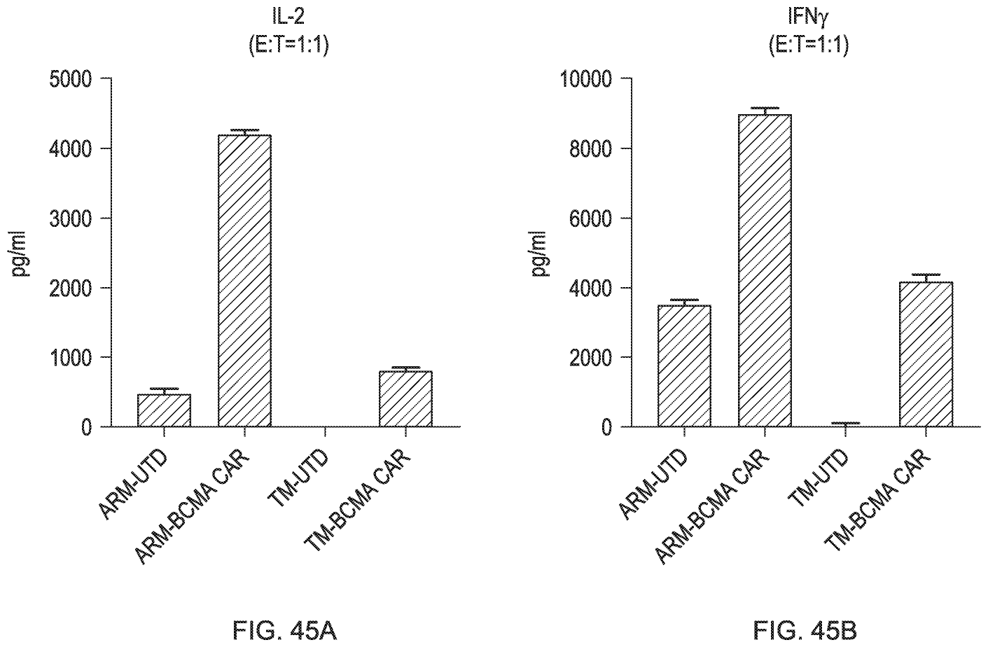

In addition to its phenotype, the final ARM-BCMA CAR cell product was also assessed for its activation in vitro. ARM-BCMA CAR and TM-BCMA CAR were thawed and co-cultured with the BCMA-expressing cell line KMS-11. Post-thaw ARM-BCMA CAR cells were rested for 24 h prior to co-culture being established. Comparing cytokine levels in the supernatants 24 hours after co-culture revealed a 5-fold increase of IL-2 and a 2-fold increase in levels of IFN-γ secreted by ARM-BCMA CAR as compared to TM-BCMA CAR as shown in FIGS. 45A and 45B. Experiments with UTD cells that underwent the ARM or TM process confirmed BCMA-specific recognition by ARM-BCMA CAR and TM-BCMA CAR. However, the higher background of IFN-γ secretion by ARM-UTD in the absence of BCMA-specific stimulation (FIG. 45B) is likely due to the activated nature of the ARM products.

In summary, the ARM process used to generate BCMA CART cells results in T cells with CAR-expression higher than that of the TM process. ARM-BCMA CAR demonstrates BCMA-specific activation in vitro and secretes higher levels of IL-2 as compared to TM-BCMA CAR, which correlates with its Tstem phenotype.

### Efficacy of ARM-BCMA CAR and TM-BCMA CAR in a Xenograft Model

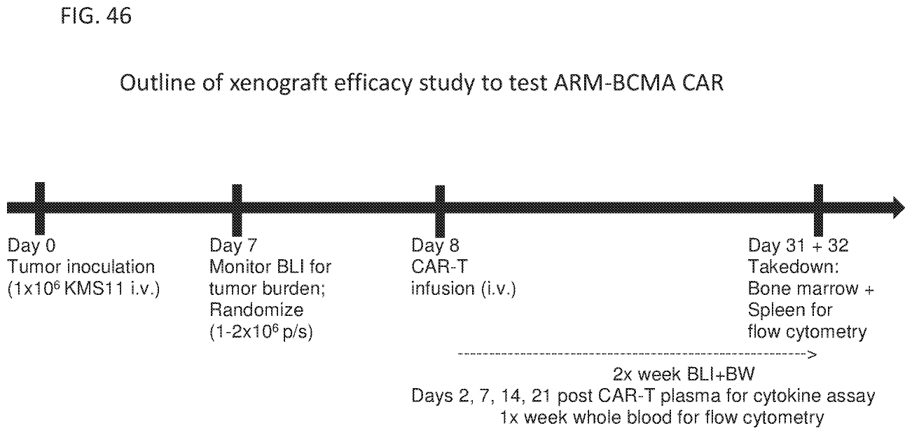

Pharmacology studies in vivo were used to guide the development of ARM-BCMA CAR. For the experiment described in FIG. 46, ARM-BCMA CAR was generated with GMP material. In parallel, TM-BCMA CAR was made using the same batch of T cells but with TM. For dose calculation using ARM-BCMA CAR, the measurement of % CAR+ at 72 h post-thaw of product was used to calculate the dose; while for TM-BCMA CAR, % CAR+ on day 9 TM products was used to calculate the dose. The efficacy of CAR-T cells generated using the different processes was evaluated in immunodeficient NSG mice (NOD-scid IL2Rg-null), which were inoculated with the MM cell line KMS-11-Luc. This tumor cell line engrafts in the bone marrow. Eight days after MM inoculation, cohorts of mice received a single infusion of CAR+ T cells. Doses were normalized to total CAR-T cells for the matched dose group. UTD T cells were prepared similarly and given as an independent group to control for allogeneic response to the tumor. The UTD dose reflected the highest total T cell dose of the respective process we could achieve for both TM and ARM.

TABLE 26
Summary of the study design for different dose groups, and
time points for blood pharmacokinetic (PK) and
plasma cytokine measurement.

| | | | Blood cellular |
|---|---|---|---|
| | | | kinetics and plasma |
| Cell | | CAR+ | cytokine post |
| process | Group/arm | product/mouse | CAR-T injection |
| | PBS | | |
| ARM | UTD | | |
| | ARM-BCMA CAR | 1.5e⁵ | |
| | ARM-BCMA CAR | 5e⁴ | |
| | ARM-BCMA CAR | 1e⁴ | |
| TM | UTD | | |
| | TM-BCMA CAR | 5e⁵ | |
| | TM-BCMA CAR | 1.5e⁵ | |
| | TM-BCMA CAR | 5e⁴ | |

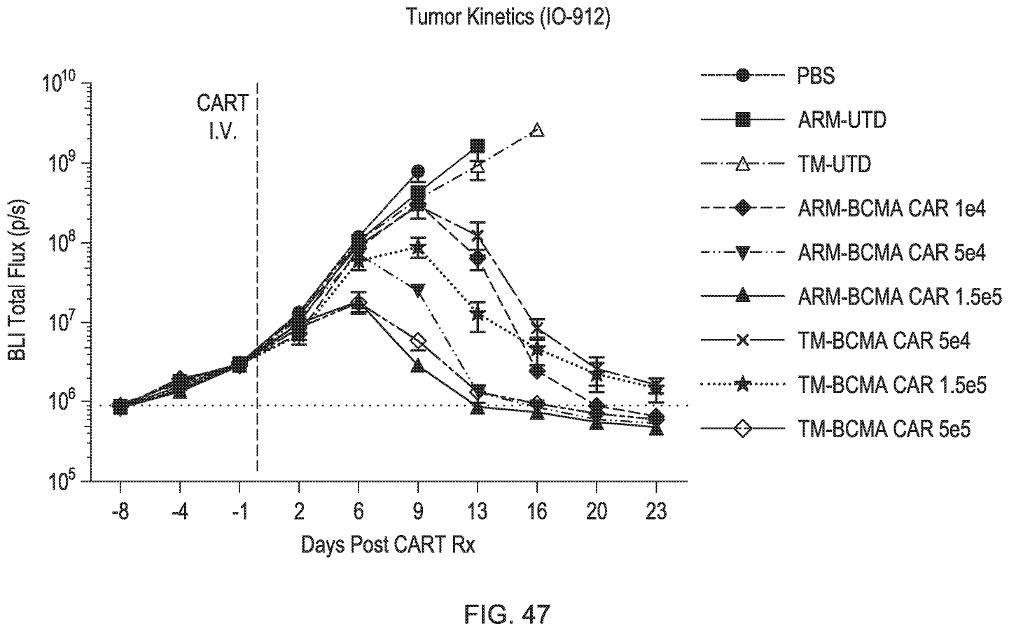

FIG. 47 is the tumor regression curve for all the groups. Both BCMA CAR-T products (ARM-BCMA CAR and TM-BCMA CAR) were able to eliminate tumor at the tested dose levels, even at the lowest dose group. Tumor-regression was induced in a dose-dependent manner. The on-set of effect in tumor-killing was delayed for about a week at the low dose group compared to the high dose group. ARM-BCMA CAR induced similar tumor regression at doses 3-5 folds lower than TM-BCMA CAR, indicating that ARM-BCMA CAR is 3-5 folds more potent compared to TM-BCMA CAR in tumor-killing.

Moreover, in this study, the efficacy of TM-BCMA CAR* was also evaluated. TM-BCMA CAR* and ARM-BCMA CAR expressed the same anti-BCMA CAR, but were manufactured using different processes: the TM process and the ARM process, respectively. The results demonstrated that ARM-BCMA CAR induced similar tumor regression at doses 1-5 folds lower than TM-BCMA CAR*

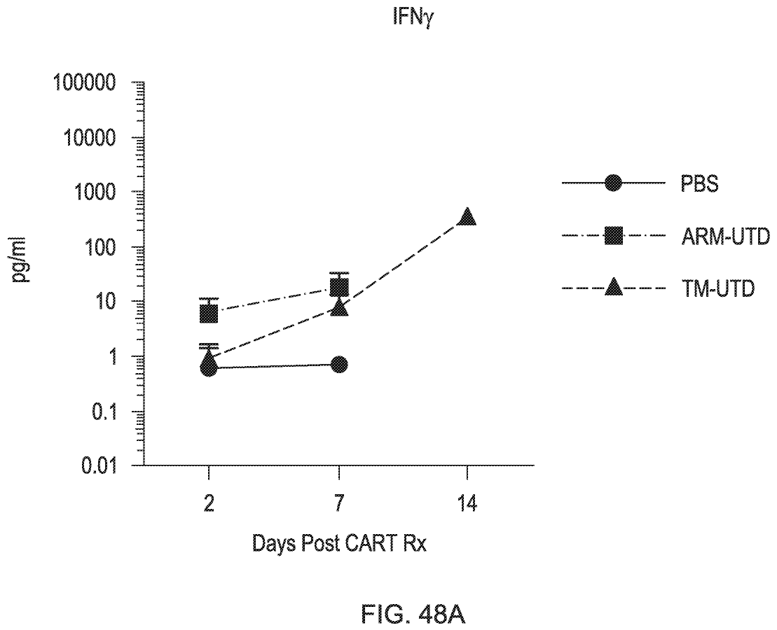

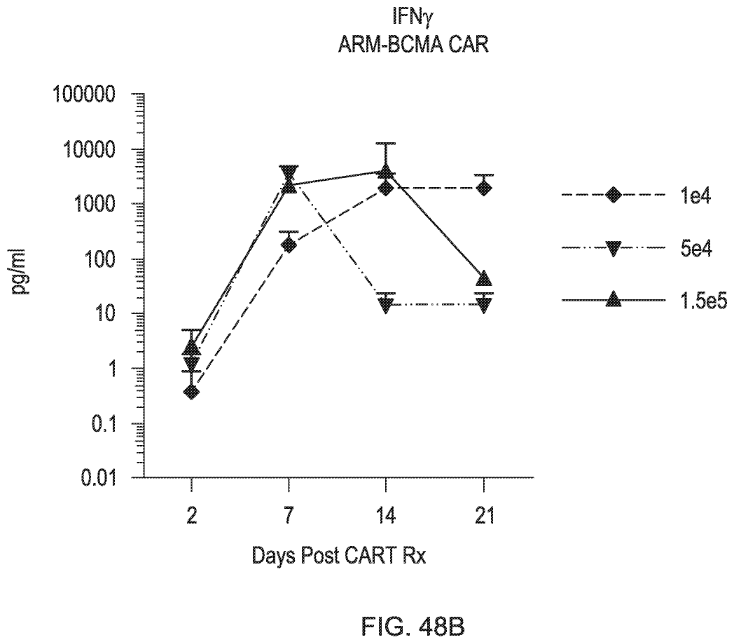

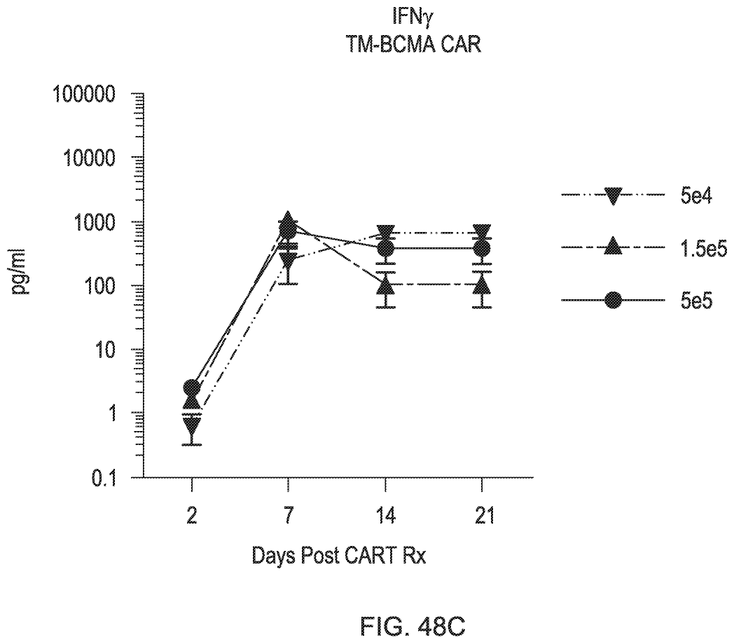

All mice were bled at day 2, 7, 14, and 21 post CAR-T therapy to measure plasma IFN-γ (FIGS. 48A-48C). No early peak was observed and all groups showed very low level of circulating IFN-γ (<10 pg/ml) at day 2. Peaks for all the groups were observed within 14 days post CAR-T dose. However, IFN-γ levels were 3.5-fold higher for ARM-BCMA CAR compared to TM-BCMA CAR. ARM-UTD groups produce little or no IFN-γ at day 2 and day 7 prior to study termination. IFN-γ declined in the higher dose groups at day 21, when compared to the ARM-BCMA CAR 1e4 and TM-BCMA CAR 5e4 groups as the CAR+ T cells were still expanding with delayed tumor inhibition in these two groups.

### In Vivo ARM-BCMA CAR Cellular Kinetics

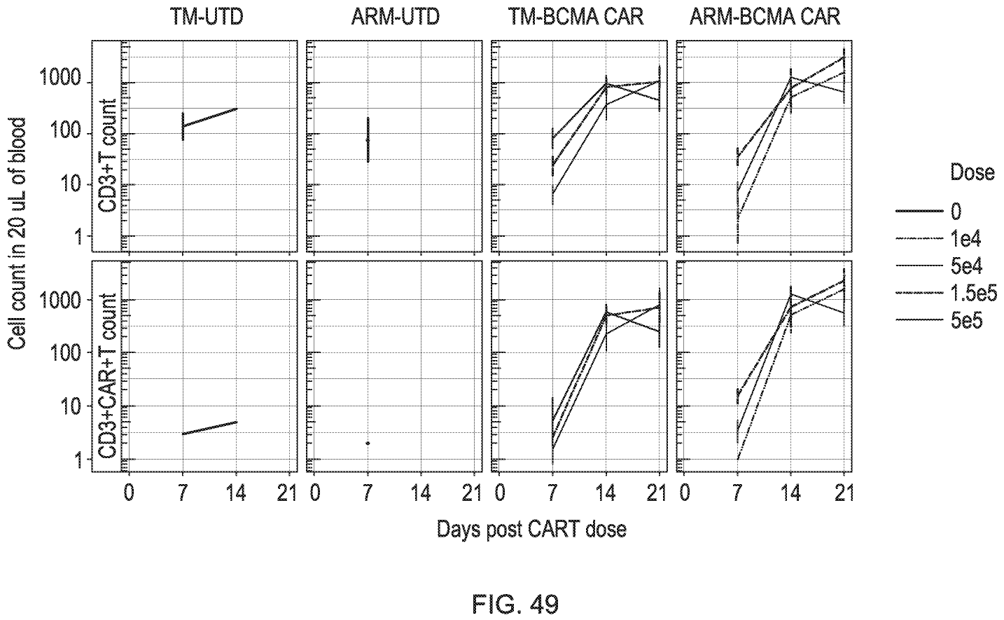

As part of this pharmacology study to assess efficacy in NSG mice, the expansion of peripheral blood CAR-T cells was analyzed by FACS up to 3 weeks after infusion. Both CD3+ T cell and CAR+ T cell expansion were observed in all CAR-T treated groups. There was no clear dose-dependent expansion for ARM-BCMA CAR or TM-BCMA CAR with respect to Cmax or AUC0-21d. The peak of cellular expansion for ARM-BCMA CAR or MTV273 was not achieved within 21 days. However, TM-BCMA CAR at dose group of 5e5 and ARM-BCMA CAR at dose group of 0.5e5 achieved apparent peak expansion at day 14 (FIG. 49). Comparing the expansion of ARM-BCMA CAR with that of TM-BCMA CAR in 21 days, both Cmax and AUC0-21d of ARM-BCMA CAR were 2 to 3 times higher.

### Example 14: Manufacturing BCMA CART Cells Using the Activated Rapid Manufacturing (ARM) Process Using IL-15 or hetIL-15 (IL-15/sIL-15Ra)

The ARM process of BCMA CART cells initiates with the preparation of the media as outlined in Table 25.

Cryopreserved leukapheresis product is used as the starting material and is processed for T cell enrichment. When available, the apheresis paper work is utilized to define the T cell percentage. In the absence of the T cell percentage data on the apheresis paperwork, the sentinel vial testing is performed on incoming cryopreserved leukapheresis products to obtain T cell percentage target for the apheresis. The results for the T cell percentage determine how many bags are thawed on Day 0 of the ARM process.

Cryopreserved leukapheresis is thawed, washed, and then undergoes T cell selection and enrichment using CliniMACS® microbead technology. Viable nucleated cells (VNCs) are activated with TransACT (Miltenyi) and transduced with a lentiviral vector encoding the CAR. The viable cells selected with the Miltenyi microbeads are seeded into the centricult on the Prodigy®, which is a non-humidified incubation chamber. While in culture, the cells are suspended in Rapid media, which is an OpTmizer™ CTS™ based medium that contains the CTS™ Supplement (ThermoFisher), Glutamax, IL-15 or hetIL-15 (IL-15/sIL-15Ra), and 2% Immune cell serum replacement amongst its components to promote T cell activation and transduction. Lentiviral transduction is performed once on the day of seeding after the TransACT has been added to the diluted cells in the culture media. Lentiviral vector will be thawed immediately prior to use on day of seeding for up to 30 minutes at room temperature.

From the start of the process on Day 0 to the initiation of the culture wash and harvest, BCMA CART cells are cultured for 20-28 hours from seeding. Following culture, the cell suspension undergoes two culture washes and one harvest wash within the centricult chamber (Miltenyi Biotech).

After the harvest wash on the CliniMACS® Prodigy® on day 1, the cell suspension is sampled to determine viable cell count and viability. Cell suspension is then transferred to a centrifuge to be pelleted manually. The supernatant is removed, and the cell pellet is re-suspended in CS10 (BioLife Solution), resulting in a product formulation with a final DMSO concentration of ˜10.0%. The viable cell count is formulated at the end of harvest for dosing. The doses are then distributed into individual cryobags and analytical sampling into cryovials.

Cryopreserved products are stored in monitored LN2 storage tanks, in a secure, limited access area until final release and shipping.

In some embodiments, IL-15 or hetIL-15 used in the OpTmizer™ CTS™ based medium can be replaced with IL-6 or IL-6/sIL-6Ra.

### Example 15: Manufacturing CD19 CART Cells Using the Activated Rapid Manufacturing (ARM) Process

The ARM process of CD19 CART cells initiates with the preparation of the media as outlined in Table 25.

Cryopreserved leukapheresis product is used as the starting material and is processed for T cell enrichment. When available, the apheresis paper work is utilized to define the T cell percentage. In the absence of the T cell percentage data on the apheresis paperwork, the sentinel vial testing is performed on incoming cryopreserved leukapheresis products to obtain T cell percentage target for the apheresis. The results for the T cell percentage determine how many bags are thawed on Day 0 of the ARM process.

Cryopreserved leukapheresis is thawed, washed, and then undergoes T cell selection and enrichment using CliniMACS® microbead technology. Viable nucleated cells (VNCs) are activated with TransACT (Miltenyi) and transduced with a lentiviral vector encoding the CAR. The viable cells selected with the Miltenyi microbeads are seeded into the centricult on the Prodigy®, which is a non-humidified incubation chamber. While in culture, the cells are suspended in Rapid media, which is an OpTmizer™ CTS™ based medium that contains the CTS™ Supplement (ThermoFisher), Glutamax, IL-2 and 2% Immune cell serum replacement amongst its components to promote T cell activation and transduction. Lentiviral transduction is performed once on the day of seeding after the TransACT has been added to the diluted cells in the culture media. Lentiviral vector will be thawed immediately prior to use on day of seeding for up to 30 minutes at room temperature.

From the start of the process on Day 0 to the initiation of the culture wash and harvest, CD19 CART cells are cultured for 20-28 hours from seeding. Following culture, the cell suspension undergoes two culture washes and one harvest wash within the centricult chamber (Miltenyi Biotech).

After the harvest wash on the CliniMACS® Prodigy® on day 1, the cell suspension is sampled to determine viable cell count and viability. Cell suspension is then transferred to a centrifuge to be pelleted manually. The supernatant is removed, and the cell pellet is re-suspended in CS10 (BioLife Solution), resulting in a product formulation with a final DMSO concentration of ˜10.0%. The viable cell count is formulated at the end of harvest for dosing. The doses are then distributed into individual cryobags and analytical sampling into cryovials. Cryopreserved products are stored in monitored LN2 storage tanks, in a secure, limited access area until final release and shipping.

In some embodiments, IL-2 used in the OpTmizer™ CTS™ based medium can be replaced with IL-15, hetIL-15 (IL-15/sIL-15Ra), IL-6, or IL-6/sIL-6Ra.

### Example 16—CAR T-Cell Activation with Multispecific Binding Molecules

A variety of configurations of bispecific antibodies and multimer conjugates thereof were generated. Schema for these conjugates are provided in FIGS. 51A-51B (Constructs 1-17, also referred to as F1 to F17).

To characterize T cell activation and lentiviral transduction, T cells were seeded at varying concentrations into a 96 well plate in supplemented media. Reagents comprising Constructs 1 through 17 in FIGS. 51A-51B at various concentrations were used to stimulate T cells for 30 minutes. Miltenyi TransAct at the optimal titrated concentration was used as a positive control. The stimulated T cells were then treated with lentivirus comprising a vector encoding a CD19 CAR at an MOI targeting 20% transduction. After culturing the cells, 75% of the culture was removed for FACS analysis. The remaining 25% was cultured and analyzed by FACS once more. The cells were analyzed by Celigo imaging, IFN gamma and IL-2 analysis of the supernatant, and FACS to assess successful CD19 CAR transduction. FIG. 52 shows the resulting T cell clustering.

### Example 17—Characterization of CAR T-Cells Activated with Multispecific Binding Molecules

T cells isolated from three different donors were seeded into a 96 well plate in supplemented media. Reagents comprising Constructs 1 through 17 in FIGS. 51A-51B at various concentrations were used to stimulate T cells. Miltenyi TransAct at the optimal titrated concentration was used as a positive control. The stimulated T cells were then treated with lentivirus comprising a vector encoding a CD19 CAR at an MOI targeting 20% transduction. Supernatants were removed 24 hours after stimulation for Meso Scale Discovery (MSD). For CAR expression measurement, the cells were taken 3.5 days post virus addition. The cells were analyzed by Celigo imaging, 10-plex cytokine analysis of the supernatant, and FACS to assess successful CD19 CAR transduction.

Greater than 70% cell viability was seen in the majority of constructs, although Formats 1 and 10 showed reduced viability under some conditions. Constructs 1-5, 7, 12, 13, 15, and 16 showed greater than 20% CAR transduction, averaged across three donors (FIG. 54). Multimeric constructs showed bell-shaped transduction. Reduced transduction was seen in T cells stimulated with CD3 only constructs, Constructs 14 and 17. The day 1 MSD data is provided in FIG. 53.

### Example 18—In Vitro Assessment of CAR T-Cells Manufactured with Multispecific Binding Molecules

T cells were activated with reagents comprising Constructs 1, 2, 3, 4, 5, 7, 12, 13, 15, 16 or TransAct at various concentrations and transduced with a lentivirus comprising a vector encoding a CD19 CAR at an MOI targeting 20% transduction for 24 hours. The cells were washed, phenotyped, and frozen. T cells were later thawed and assessed for recovery and viability on the same day, post-thaw. Post-thaw, T cells were analyzed for phenotype and CAR transduction, and cytokine profiles and killing assay readouts in Nalm6 WT and Nalm6 CD19 KO were obtained.

In one study, the cells were washed and half of the cells were taken for flow staining and half of the cells were plated back and incubated for 3 days. The cells were stained on the fourth day for CAR expression. In other studies, the cells were thawed later and plated with luciferized target cells at CART cells: Target cells ratios between 0.6:1 and 5:1, for 24 hours. For the study shown in FIGS. 57A-57B, a CART cell: Target cell ration of 2.5:1 was used. After 24 hours, the luciferase signal was measured as a proxy for target cell viability. This was used to calculate the effective killing ability of the CAR T cells.

As show in FIGS. 55, F1, F3, F5, and F4, all of which have a ligand valency of 2, showed higher activity than multimer constructs. CD-2 targeting constructs showed enhanced transduction across the 0.1 to 10 μg/mL concentrations. FIGS. 56A-56D further demonstrate that the resulting CAR T-cells show specific killing of CD19+ cells. Per FIGS. 57A-57B, the resulting CAR T-cells also secrete cytokines in an antigen specific manner.

### Example 19—In Vivo Assessment of CAR T-Cells Manufactured with Multispecific Binding Molecules

T cells from two donors were activated with reagents comprising Constructs 3 and 4 or TransAct and transduced with a lentivirus comprising a vector encoding CAR at an MOI of 1, targeting 20% transduction for 24 hours. The cells were washed, phenotyped, assessed for cytokine production, and frozen. T cells were later thawed and assessed for recovery and viability on the same day, post-thaw. Post-thaw, T cells were cultured for 3 additional days and analyzed for CAR transduction.

Anti-tumor activity of a set of CD19 targeting CAR T cells activated with Construct 3, 4 or TransAct was also assessed in vivo in a NALM6 xenograft model.

Details of the model and protocol are provided below.

Cell Line—Nalm6 (RRID: CVCL_0092) is a human acute lymphoblastic leukemia (ALL) cell line. Cells were grown in RPMI medium containing 10% fetal bovine serum in suspension. These cells persist and expand in mice when implanted intravenously. Cells were modified to express luciferase, so that tumor cell growth can also be monitored by imaging the mice after they have been injected with the substrate Luciferin.

Mice—6 week old NSG (NOD.Cg-PrkdcscidIl2rgtm1 Wjl/SzJ) mice were received from the Jackson Laboratory

Protocol—Nalm6 cells were injected into the mice. Nalm6 cells endogenously express CD19 and thus, are used to test the efficacy of CD19-directed CAR T cells in vivo. Mice were dosed after tumor implantation, with CAR T cells for the treatment of Nalm6. The CARTs were injected into the mice. Five mice per group were treated with PBS alone (PBS), untransduced T cells treated with TransAct, F3 or F4 STARTERS, or CAR T cells manufactured using F3, F4 or TransAct. All cells were prepared in parallel. The health status of the mice was monitored daily, including twice weekly body weight measurements. The percent change in body weight was calculated as (BWcurrent−BWinitial)/(BWinitial)×100%. Tumors were monitored 2 times weekly by imaging the mice.

As shown in FIG. 58, the resulting CD19 CAR T-cells were able to clear high in vivo tumor burdens and were comparable to TransAct for both Constructs 3 (F3) and 4 (F4). The CD19 CAR T-cells generated with Constructs 3 (F3) and 4 (F4) were also found to undergo expansion in vivo (FIG. 59).

In one study, cryopreserved donor cells were thawed into opTmizer media and plated. Vector and stimulation reagent were added and incubated for 24 hours. The cells were washed and 50% of the cells were cryopreserved, 25% were stained for flow and 25% of the cells were plated back and incubated for 3 days. The cells were stained on the fourth day for CAR expression. The cryopreserved portion of the cells were thawed later into media and counted. Then they were resuspended in PBS and injected into mice, with previously injected luciferized tumor. The luciferase signal and the number of CART cells in peripheral blood were measured every week for efficacy.

As shown in FIGS. 60A-60D, CART cells generated using Construct F3 or F4 showed anti-tumor activity and in vivo expansion.

### Example 20—In Vivo Efficacy of CAR T-Cells Manufactured with Tet2 shRNA

Using either the expansion or expansion-less manufacturing processes described herein, CAR T-cells are generated—with or without the introduction of Tet2 shRNA. In some iterations, the Tet2 shRNA is provided in the same vector as the CAR. In some iterations, the Tet2 shRNA is provided in a different vector from the CAR; in these instances, the Tet2 shRNA vector further comprises a detectable tag. In some iterations, the CAR is CD19, BCMA, CD20, CD22, CLL-1, or EGFRvIII specific. The CAR T-cells are then frozen. CAR T-cells are thawed and administered to a TMD8 mouse model. Mice are measured and weighed twice a week after tumor implantation. Plasma samples from the mice are analyzed via FACS. Bone marrow, spleen, and tumor are collected for T-cell phenotyping, and cytokine production is measured. CAR T-cells manufactured with Tet2 shRNA are found to have better anti-tumor effect and proliferation compared to the controls.

### Example 21—Boosting Transduction

Using the expansion-less manufacturing process described herein and further adding vectofusin-1, F108, or lentiboost during transduction, CART cells were generated and analyzed for % transduction at various concentrations and time points. F108 was found to boost lentiviral transduction. Additional reagents F68, F127, protamine sulfate, and polybrene are tested in the same process. In some iterations, the CAR encoded in the vector for transduction is CD19, BCMA, CD20, CD22, CLL-1, or EGFRvIII specific.

### Example 22—Second Generation Stimulatory Constructs

This example describes characterization of second generation stimulatory constructs. As shown in FIG. 61A, constructs were generated to test binders targeting different co-stimulatory molecules (e.g., CD25, IL6Rb, CD27, 41BB, ICOS, or CD2). In addition, different anti-CD3 binders (binders based on ANTI-CD3 (1) or ANTI-CD3 (2)) were also compared. All the second generation stimulatory constructs have the configuration shown in FIG. 61B. The sequences of the different binders tested in this Example can be found in Table 27.

In one study, the second generation constructs were compared for transduction efficiency. TransAct was used as a control. Briefly, cryopreserved donor cells were thawed into optimizer media and plated. Vector and stimulation reagent were added and incubated for 24 hours. The cells were washed and 50% of the cells were taken for flow staining and 50% of the cells were plated back and incubated for 3 days. The cells were stained on the fourth day for CAR expression.

As shown in FIG. 62, F5 ICOS was highly effective at 0.01 μg/mL and its transduction efficiency decreased when F5 ICOS concentration was increased. F5 (the construct with the ANTI-CD3 (1)-based anti-CD3 binder) was more potent than F5 ANTI-CD3 (2) (the construct with the ANTI-CD3 (2)-based anti-CD3 binder).

### Example 23—Third Generation Stimulatory Constructs

Without wishing to be bound by theory, reducing the binding of stimulatory constructs to FcR may decrease or prevent unwanted killing of FcR-expressing cells by T cells. Third generation stimulatory constructs were generated by introducing D265A/N297A/P329A substitutions (EU numbering according to Kabat) (“DANAPA”) in the IgG1 Fc region of the stimulatory constructs. In addition, different anti-CD3 binders (binders based on ANTI-CD3 (1), ANTI-CD3 (2), or ANTI-CD3 (3)) and different anti-CD28 binders (binders based on ANTI-CD28 (1) or ANTI-CD28 (2)) were also compared (FIG. 63A). All the third generation stimulatory constructs have the configuration shown in FIG. 63B. The sequences of the different binders tested in this Example can be found in Table 27.

In one study, the third generation constructs were compared for transduction efficiency. Briefly, cryopreserved donor cells were thawed into opTmizer media and plated. Vector and stimulation reagent were added and incubated for 24 hours. The cells were washed and 50% of the cells were taken for flow staining and 50% of the cells were plated back and incubated for 3 days. The cells were stained on the fourth day for CAR expression.

As shown in FIG. 64, all the anti-CD3/CD28 or anti-CD3/CD2 constructs tested promoted CAR transduction.

In another study, the third generation constructs were compared for specific versus non-specific killing. Briefly, cryopreserved donor cells were thawed into opTmizer media and plated. Vector and stimulation reagents were added and incubated for 24 hours. The cells were washed and 50% of the cells were cryopreserved, 25% of the cells were stained for flow cytometry and 25% of the cells were plated back and incubated for 3 days. The cells were stained on the fourth day for CAR expression. The cryopreserved cells were thawed later and plated with luciferized target tumor cells and non-target tumor cells at CART cells: tumor cells ratios between 0.6:1 and 5:1, e.g., 1:5, for 24 hours. After 24 hours, the luciferase signal was measured as a proxy for target cell viability. This was used to calculate the specific and non-specific killing ability of the CAR T cells.

Silenced formats of F3 and F4 (F3 and F4 with DANAPA substitutions in the Fc region), NEG2042 and NEG2043, showed equivalent killing of target cells (FIG. 65A) but do not show non-specific killing of non-target cells (FIG. 65B). In contrast, F3 and F4 with the wild type Fc region showed non-specific killing of FcγR-expressing cells (FIG. 65B).

In another study, the third generation construct NEG2043 was used to generate cells expressing two CARs. In a first study, cryopreserved donor T cells were thawed into OpTmizer media and co-incubated for 24 hours with stimulation reagent and two vectors: a first vector encoding a CD19-targeting CAR, and a second vector encoding a BCMA-targeting CAR. After 24 hours, the cells were washed; 50% of the cells were stained for flow cytometry and 50% of the cells were replated and incubated for additional 3 days. Cells were stained for CAR expression on the fourth day. In a second study, cryopreserved donor leukapheresis was thawed, washed, and enriched for T cells using the CliniMACS Prodigy. The enriched cells were eluted into OpTmizer media. The enriched cells were co-incubated for 24 hours with stimulation reagent and a vector encoding both a CD19-targeting CAR and a CD22-targeting CAR. After 24 hours, the cells were washed; 50% of the cells were stained for flow cytometry and 50% of the cells were replated and incubated for an additional 3-6 days. Cells were stained for CAR expression on the fourth and seventh days.

F4 and the silenced format of F4 (NEG2043) were capable of expressing one or both CARs when T cells were (1) co-transduced with two CAR-encoding vectors or (2) transduced with a single vector encoding two CARs. FIG. 66A shows co-transduction of a first vector encoding a CD19-targeting CAR, and a second vector encoding a BCMA-targeting CAR. FIG. 66B shows transduction of a single vector encoding both a CD19-targeting CAR and a CD22-targeting CAR.

### Example 24—In Vitro Testing of Fc-Silenced Constructs Anti-CD3 (4)/Anti-CD28 (2) Bispecific, Anti-CD3 (2)/Anti-CD28 (2), and Anti-CD3 (4)/Anti-CD28 (1)

In one study, Fc-silenced (LALASKPA) Constructs Anti-CD3 (4)/anti-CD28 (2) bispecific (SEQ ID NO: 794 and 796), Anti-CD3 (2)/anti-CD28 (2) (SEQ ID NO: 798 and 799), and Anti-CD3 (4)/anti-CD28 (1) (SEQ ID NO: 800 and 801) were compared for transduction efficiency of T cells. Briefly, cryopreserved donor cells were thawed into OpTmizer media and plated. Vector and stimulation reagent were added and incubated for 24 hours. The cells were washed and 50% of the cells were taken for flow staining and 50% of the cells were plated back and incubated for 3 days. The cells were stained on the fourth day for CAR expression. All of the constructs tested promoted CAR transduction.

In another study, an Fc-silenced (LALASKPA) Anti-CD3 (4)/anti-CD28 (2) bispecific construct (SEQ ID NO: 794 and 796) and TransAct produced CARTs were tested for specific versus non-specific killing. Cryopreserved CD19 CARTs were thawed and plated with luciferized Nalm6 target tumor cells and FcγR-expressing PL21 non-target tumor cells at CART cells: tumor cells ratios between 0.6:1 and 5:1, e.g., 2.5:1, for 24 hours. After 24 hours, the luciferase signal was measured as a proxy for target cell viability. This was used to calculate the specific and non-specific killing ability of the CAR T cells. The Anti-CD3 (4)/anti-CD28 (2) bispecific construct (SEQ ID NO: 794 and 796) produced CARTs showed comparable specific Nalm6 killing to TransAct produced CARTs (FIG. 68A). Little non-specific killing of PL21 cells was seen, except for the highest E: T ratio (FIG. 68B).

In a third study, Fc-silenced (GADAPASK or LALAPG) Anti-CD3 (1)/anti-CD2 (1) bispecific constructs and TransAct produced CARTs were tested for non-specific killing. The two bispecific constructs are referred to as “F3 GADAPASK” (SEQ ID NOs: 816 and 673) and “F3 LALAPG” (SEQ ID NOs: 817 and 673) in FIG. 69. T cells were plated with FcγR-expressing PL21 cells at various E: T ratios for 24 hours. After 24 hours, the luciferase signal was measured as a proxy for target cell viability. Non-specific killing of PL21 cells was minimal, except for the highest E: T ratio (FIG. 69).

### Example 25—In Vivo Testing of Fc-Silenced Constructs Anti-CD3 (4)/Anti-CD28 (2) Bispecific, Anti-CD3 (2)/Anti-CD28 (2), and Anti-CD3 (4)/Anti-CD28 (1)

T cells were activated with reagents comprising an Fc-silenced (LALASKPA) Anti-CD3 (4)/anti-CD28 (2) bispecific construct (SEQ ID NO: 794 and 796), Anti-CD3 (2)/anti-CD28 (2) (SEQ ID NO: 798 and 799) construct, Anti-CD3 (4)/anti-CD28 (1) (SEQ ID NO: 800 and 801) construct, or TransAct and transduced with a lentivirus comprising a vector encoding CAR19 for 24 hours. The cells were washed, phenotyped, assessed for cytokine production, and frozen. T cells were cultured for 3 additional days and analyzed for CAR transduction. T cells were later thawed and assessed for recovery and viability post-thaw. Anti-tumor activity of a set of CD19 targeting CAR T cells activated with an Fc-silenced (LALASKPA) Anti-CD3 (4)/anti-CD28 (2) bispecific construct (SEQ ID NO: 794 and 796), Anti-CD3 (2)/anti-CD28 (2) (SEQ ID NO: 798 and 799) construct, Anti-CD3 (4)/anti-CD28 (1) (SEQ ID NO: 800 and 801) construct, or TransAct was also assessed in vivo in a TMD8 xenograft model. Further details of the model and protocol are provided below:

Cell line: TMD8 (RRID: CVCL_A442) is a human diffuse large B cell lymphoma (DLBCL) cell line. Cells were grown in alpha MEM, 10% fetal bovine serum, 1×1-glutamine and 1×NEAA in suspension culture. Cells persist and expand in mice when implanted subcutaneously.

Mice: 6 week old NSG mice were received from the Jackson Laboratory (stock number 005557).

Tumor implantation: TMD8 cells in logarithmic growth phase were harvested and washed in 50 ml falcon tubes at 1200 rpm for 5 minutes, once in growth media and then twice in cold sterile PBS. Cells were resuspended in a 1:1 ratio of PBS:Matrigel at a concentration of 50×10⁶ per ml, placed on ice, and injected in mice. Cancer cells were injected subcutaneously in 100 μl on the right flank. TMD8 cells endogenously express CD19 and thus can be used to test the efficacy of CD19-directed CAR T cells in vivo. This model grows well when implanted subcutaneously in mice, and tumor volume measurements can be performed with calipers. Upon injection of 5×10⁶ cancer cells, the tumors establish and can be accurately measured within 7 days. Within 10 days the mean tumor volume measurement is 150-200 mm³ and untreated tumors reach endpoint measurement (tumor volume equal to 10% body weight) by 20-30 days. Anti-tumor activities of therapeutic agents are often tested once tumors are fully engrafted. Thus, there is a large window with these models during which the anti-tumor activity of CAR T cells can be observed.

CAR T cell dosing: Mice were dosed 10 days after tumor implantation, with 0.25×10⁶ anti-CD19 CAR T cells made with an Anti-CD3 (4)/anti-CD28 (2) bispecific construct (SEQ ID NO: 794 and 796), Anti-CD3 (2)/anti-CD28 (2) (SEQ ID NO: 798 and 799) construct, Anti-CD3 (4)/anti-CD28 (1) (SEQ ID NO: 800 and 801) construct, or TransAct as an activation reagent. Cells were partially thawed in a 37° C. water bath and then completely thawed by the addition of 1 ml of warmed growth media. The thawed cells were transferred to a 50 ml falcon tube and adjusted to a final volume of 12 ml with growth media. The cells were washed twice and spun at 300 g for 10 minutes and then counted by hemocytometer. T cells were then resuspended at respective concentrations in cold PBS and kept on ice until the mice were dosed. The CARTs were injected intravenously via the tail vein in 200 μl, for a dose of 0.25×10⁶ CAR T cells. 5 mice per group were either treated with 200 μl of PBS alone (PBS), untransduced T cells manufactured with different Fc-silenced (LALASKPA) Constructs Anti-CD3 (4)/anti-CD28 (2) bispecific (SEQ ID NO: 794 and 796), Anti-CD3 (2)/anti-CD28 (2) (SEQ ID NO: 798 and 799), Anti-CD3 (4)/anti-CD28 (1) (SEQ ID NO: 800 and 801). All cells were prepared from the same donor in parallel.

Animal monitoring: The health status of the mice was monitored daily, including 2-3 times weekly body weight measurements. The percent change in body weight was calculated as (BWcurrent−BWinitial)/(BWinitial)×100%. Tumors were monitored 2-3 times weekly by caliper measurements of the tumor.

Following tumor cell implantation on day 0, tumor-bearing mice were randomized into treatment groups and CAR T cells were administered intravenously via the lateral tail vein on day 10 after tumor implantation. Tumor growth and animal health were monitored until animals achieved endpoint. In this TMD8 model, mice which received PBS or untransduced T cells were euthanized around day 23, when tumors had reached maximum allowed volume. All other groups were euthanized on day 37. The mean tumor volume for all treatment groups is plotted in the figure. The PBS treatment group, which did not receive any T cells, demonstrates exponential TMD8 tumor growth kinetics. The UTD treatment group received untransduced T cells and served as a T cell control to show the non-specific response of human donor T cells in this model and showed continuous tumor progression throughout this study. As shown in FIG. 67, the Fc-silenced (LALASKPA) Anti-CD3 (4)/anti-CD28 (2) bispecific construct (SEQ ID NO: 794 and 796), Anti-CD3 (2)/anti-CD28 (2) (SEQ ID NO: 798 and 799) construct, Anti-CD3 (4)/anti-CD28 (1) (SEQ ID NO: 800 and 801) construct C-manufactured CAR-T cells showed tumor control comparable to TransAct-produced CARTs.

### Example 26—Patient Cell Transduction with Fc-Silenced (LALASKPA) Constructs Anti-CD3 (4)/Anti-CD28 (2) Bispecific, Anti-CD3 (2)/Anti-CD28 (2), and Anti-CD3 (4)/Anti-CD28 (1)

In one study, the Fc-silenced (LALASKPA) Anti-CD3 (4)/anti-CD28 (2) bispecific construct (SEQ ID NO: 794 and 796), Anti-CD3 (2)/anti-CD28 (2) (SEQ ID NO: 798 and 799) construct, and Anti-CD3 (4)/anti-CD28 (1) (SEQ ID NO: 800 and 801) construct were compared for transduction efficiency using patient-derived T cells. TransAct was used as a control. Briefly, cryopreserved DLBCL patient PBMCs were acquired from Discovery Life Sciences. Cells were thawed into OpTmizer media containing DNAse and T cells were purified using an EasySep Pan T negative selection kit (Stemcell Tech). CD19 CAR vector and stimulation reagent were added and incubated for 24 hours. Cells were washed and characterized by flow cytometry for CAR expression. The Fc-silenced (LALASKPA) Anti-CD3 (4)/anti-CD28 (2) bispecific construct (SEQ ID NO: 794 and 796), Anti-CD3 (2)/anti-CD28 (2) (SEQ ID NO: 798 and 799) construct, and Anti-CD3 (4)/anti-CD28 (1) (SEQ ID NO: 800 and 801) construct were able to facilitate transduction of DLBCL patient cells.

### Example 27—Manufacturing CD19 CART Cells and BCMA CART Cells in Large Scale Studies

The feasibility of manufacturing CD19 CART cells and BCMA CART cells was tested at large scale using three unique donors.

Briefly, cryopreserved leukapheresis was thawed, washed, and underwent T cell selection and enrichment using CliniMACS®. Microbead technology. The enriched viable nucleated cells (VNCs) were activated with either TransAct (Miltenyi) or Fc-silenced (LALASKPA) Anti-CD3 (4)/anti-CD28 (2) bispecific (SEQ ID NO: 794 and 796), and concurrently transduced with lentiviral vector encoding either CD19 CAR or BCMA CAR. The cells were seeded into the CentriCult on the Prodigy®, which is a non-humidified incubation chamber. The cells were cultured in Rapid media, an OpTmizer™ CTS™ based medium that contains the CTS™ Supplement (ThermoFisher), Glutamax, IL-2 and 2% Immune cell serum replacement amongst its components to promote T cell activation and transduction. 20-28 hours after seeding, the cells were washed three times within the CentriCult chamber (Miltenyi Biotec) with HSA-containing buffer and harvested. The harvested cells were aliquoted for cryopreservation, immediate flow cytometry analysis, or further cultured in vitro and analyzed for CAR expression. Use of either TransAct or Anti-CD3 (4)/anti-CD28 (2) bispecific (SEQ ID NO: 794 and 796) resulted in CARTs with comparable expression of BCMA CAR (FIG. 70A) and similar final product phenotypes (FIGS. 70B and 70C). Notably, the T cell memory phenotype of the manufactured CART cells (Day 1 (“D1”) samples in FIG. 70B) is similar to the T cell memory phenotype of the input material (Day 0 (“D0”) samples in FIG. 70B). The CD19 CAR study using cells from two different donors showed similar results (FIGS. 71A-71F). In conclusion, this study shows that Fc-silenced (LALASKPA) anti-CD3/anti-CD28 bispecific antibody can be used to manufacture CART cells while maintaining the T cell memory phenotype of the input materials.

### EQUIVALENTS

The disclosures of each and every patent, patent application, and publication cited herein are hereby incorporated herein by reference in their entirety. While this invention has been disclosed with reference to certain embodiments, it is apparent that further embodiments and variations of this invention may be devised by others skilled in the art without departing from the true spirit and scope of the invention. The appended claims are intended to be construed to include all such embodiments and equivalent variations.

What is claimed is:

1. A method of making a population of cells (for example, T cells) that express a chimeric antigen receptor (CAR), the method comprising:
(i) contacting (for example, binding) a population of cells (for example, T cells, for example, T cells isolated from a frozen or fresh leukapheresis product) with a multispecific binding molecule comprising (A) an anti-CD3 binding domain, (B) a costimulatory molecule binding domain (e.g., an anti-CD2 binding domain or an anti-CD28 binding domain), and (C) an Fc region comprising:
a L234A, L235A, S267K, and P329A mutation (LALASKPA), numbered according to the EU numbering system;
a L234A, L235A, and P329G mutation (LALAPG), numbered according to the EU numbering system;
a G237A, D265A, P329A, and S267K mutation (GADAPASK), numbered according to the EU numbering system;
a L234A, L235A, and G237A mutation (LALGA), numbered according to the EU numbering system;
a D265A, P329A, and S267K mutation (DAPASK), numbered according to the EU numbering system;
a G237A, D265A, and P329A mutation (GADAPA), numbered according to the EU numbering system; or
a L234A, L235A, and P329A mutation (LALAPA), numbered according to the EU numbering system;
(ii) contacting the population of cells (for example, T cells) with a nucleic acid molecule (for example, a DNA or RNA molecule) encoding the CAR, thereby providing a population of cells (for example, T cells) comprising the nucleic acid molecule, and
(iii) harvesting the population of cells (for example, T cells) for storage (for example, reformulating the population of cells in cryopreservation media) or administration, wherein:
(a) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and
step (iii) is performed no later than 30 (for example, 26) hours after the beginning of step (i), for example, no later than 22, 23, 24, 25, 26, 27, 28, 29, or 30 hours after the beginning of step (i), for example, no later than 24 hours after the beginning of step (i),
(b) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and
step (iii) is performed no later than 30 hours after the beginning of step (ii), for example, no later than 22, 23, 24, 25, 26, 27, 28, 29, or 30 hours after the beginning of step (ii), or
(c) the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i),
optionally wherein the nucleic acid molecule in step (ii) is on a viral vector, optionally wherein the nucleic acid molecule in step (ii) is an RNA molecule on a viral vector, optionally wherein step (ii) comprises transducing the population of cells (for example, T cells) with a viral vector comprising a nucleic acid molecule encoding the CAR.

2. The method of claim 1, wherein:
(i) the anti-CD3 binding domain, e.g., an anti-CD3 scFv, is situated N-terminal of the costimulatory molecule binding domain, e.g., an anti-CD2 Fab or an anti-CD28 Fab; or
(ii) the anti-CD3 binding domain, e.g., an anti-CD3 scFv, is situated C-terminal of the costimulatory molecule binding domain, e.g., an anti-CD2 Fab or an anti-CD28 Fab.

3. The method of claim 1 or 2, wherein the Fc region comprises a CH2.

4. The method of any one of claims 1-3, wherein the Fc region comprises a CH3.

5. The method of any one of claims 1-4, wherein the anti-CD3 binding domain is situated C-terminal of the Fc region.

6. The method of any one of claims 1-4, wherein the anti-CD3 binding domain is situated N-terminal of the Fc region.

7. The method of any one of claims 1-6, wherein the Fc region is situated between the anti-CD3 binding domain and the costimulatory molecule binding domain.

8. The method of any one of claim 1-4 or 6, wherein the multispecific binding molecule comprises:
(i) a first polypeptide comprising from N-terminal to C-terminal: VH of the anti-CD3 binding domain, VL of the anti-CD3 binding domain, VH of the costimulatory molecule binding domain, CH1, CH2, and CH3; and
(ii) a second polypeptide comprising from N-terminal to C-terminal: VL of the costimulatory molecule binding domain and CL.

9. The method of any one of claim 1-5 or 7, wherein the multispecific binding molecule comprises:
(i) a first polypeptide comprising from N-terminal to C-terminal: VH of the costimulatory molecule binding domain, CH1, CH2, CH3, VH of the anti-CD3 binding domain, and VL of the anti-CD3 binding domain; and
(ii) a second polypeptide comprising from N-terminal to C-terminal: VL of the costimulatory molecule binding domain and CL.

10. The method of any one of claim 1-4 or 6, wherein the multispecific binding molecule comprises:
(i) a first polypeptide comprising from N-terminal to C-terminal: VH of the costimulatory molecule binding domain, CH1, VH of the anti-CD3 binding domain, VL of the anti-CD3 binding domain, CH2, and CH3; and
(ii) a second polypeptide comprising from N-terminal to C-terminal: VL of the costimulatory molecule binding domain and CL.

11. The method of any one of claims 1-10, wherein the anti-CD3 binding domain comprises an scFv and the costimulatory molecule binding domain is part of a Fab fragment.

12. The method of any one of claims 1-11, wherein the anti-CD3 binding domain comprises:
(i) a variable heavy chain region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3 of an anti-CD3 antibody molecule of Table 27 (for example the anti-CD3 (1), anti-CD3 (2), anti-CD3 (3), or anti-CD3 (4)); and/or
(ii) the amino acid sequence of any VH and/or VL region of an anti-CD3 antibody molecule provided in Table 27 (for example the anti-CD3 (1), anti-CD3 (2), anti-CD3 (3), or anti-CD3 (4)), or an amino acid sequence at least 95% identical thereto.

13. The method of any one of claims 1-12, wherein the costimulatory molecule binding domain is an anti-CD2 binding domain, optionally wherein the anti-CD2 binding domain comprises:
(i) a VH comprising a HCDR1, a HCDR2, and a HCDR3, and a VL comprising a LCDR1, a LCDR2, and a LCDR3 of an anti-CD2 antibody molecule of Table 27 (for example the anti-CD2 (1)); and/or
(ii) the amino acid sequence of any VH and/or VL region of an anti-CD2 antibody molecule provided in Table 27 (for example the anti-CD2 (1)), or an amino acid sequence at least 95% identical thereto.

14. The method of any one of claims 1-13, wherein the costimulatory molecule binding domain is an anti-CD28 binding domain, optionally wherein the anti-CD28 binding domain comprises:
(i) a VH comprising a HCDR1, a HCDR2, and a HCDR3, and a VL comprising a LCDR1, a LCDR2, and a LCDR3 of an anti-CD28 antibody molecule of Table 27 (for example the anti-CD28 (1) or anti-CD28 (2)); and/or
(ii) the amino acid sequence of any VH and/or VL region of an anti-CD28 antibody molecule provided in Table 27 (for example the anti-CD28 (1) or anti-CD28 (2)), or an amino acid sequence at least 95% identical thereto.

15. The method of any one of claims 1-14, wherein the anti-CD3 binding domain comprises:
(i) an scFv;
(ii) a VH linked to a VL by a peptide linker, e.g., a glycine-serine linker, e.g., a (G₄S)₄ linker; or
(iii) a VH and a VL, wherein the VH is N-terminal of the VL.

16. The method of any one of claims 1-15, wherein the costimulatory molecule binding domain is part of a Fab fragment, e.g., a Fab fragment that is part of a polypeptide sequence that comprises the Fc region.

17. The method of any one of claims 1-16, wherein the anti-CD3 binding domain is situated N-terminal of the costimulatory molecule binding domain, optionally wherein the anti-CD3 binding domain is linked to the costimulatory molecule binding domain by a peptide linker, e.g., a glycine-serine linker, e.g., a (G₄S)₄ linker.

18. The method of any one of claim 1-7 or 9-17, wherein the anti-CD3 binding domain is situated C-terminal of the costimulatory molecule binding domain.

19. The method of claim 18, wherein:
(i) the Fc region is situated between the anti-CD3 binding domain and the costimulatory molecule binding domain; and/or
(ii) the multispecific binding molecule comprises one or both of a CH2 and a CH3, optionally wherein the anti-CD3 binding domain is linked to the CH3 by a peptide linker, e.g., a glycine-serine linker, e.g., a (G₄S)₄ linker.

20. The method of claim 18, wherein:
(i) the multispecific binding molecule comprises a CH2, and the anti-CD3 binding domain is situated N-terminal of the CH2;
(ii) the anti-CD3 binding domain is linked to a CH1 by a peptide linker, e.g., a glycine-serine linker, e.g., a (G₄S)₂ linker; and/or
(iii) the anti-CD3 binding domain is linked to a CH2 by a peptide linker, e.g., a glycine-serine linker, e.g., a (G₄S)₄ linker.

21. The method of any one of claims 1-20, wherein step (i) increases the percentage of CAR-expressing cells in the population of cells from step (iii), for example, the population of cells from step (iii) shows a higher percentage of CAR-expressing cells (for example, at least 10, 20, 30, 40, 50, or 60% higher), compared with cells made by an otherwise similar method without step (i).

22. The method of any one of claims 1-21, wherein:
(a) the percentage of naïve cells, for example, naïve T cells, for example, CD45RA+ CD45RO− CCR7+ T cells, in the population of cells from step (iii) is the same as or differs by no more than 5 or 10% from the percentage of naïve cells, for example, naïve T cells, for example, CD45RA+ CD45RO− CCR7+ cells, in the population of cells at the beginning of step (i);
(b) the percentage of naïve cells, for example, naïve T cells, for example, CD45RA+ CD45RO− CCR7+ T cells, in the population of cells from step (iii) is increased by, for example, at least 1.2, 1.4, 1.6, 1.8, 2.0, 2.2, 2.4, 2.6, 2.8, or 3-fold, as compared to the percentage of naïve cells, for example, naïve T cells, for example, CD45RA+ CD45RO− CCR7+ cells, in the population of cells at the beginning of step (i);
(c) the percentage of CAR-expressing naïve T cells, for example, CAR-expressing CD45RA+ CD45RO− CCR7+ T cells in the population of cells increases during the duration of step (ii), for example, increases by, for example, at least 30, 35, 40, 45, 50, 55, or 60%, between 18-24 hours after the beginning of step (ii); or
(d) the percentage of naïve cells, for example, naïve T cells, for example, CD45RA+ CD45RO− CCR7+ T cells, in the population of cells from step (iii) does not decrease, or decreases by no more than 5 or 10%, as compared to the percentage of naïve cells, for example, naïve T cells, for example, CD45RA+ CD45RO− CCR7+ cells, in the population of cells at the beginning of step (i).

23. The method of any one of claims 1-22, wherein:
(a) the population of cells from step (iii) shows a higher percentage of naïve cells, for example, naïve T cells, for example, CD45RA+ CD45RO− CCR7+ T cells (for example, at least 10, 20, 30, or 40% higher), compared with cells made by an otherwise similar method in which step (iii) is performed more than 26 hours after the beginning of step (i), for example, more than 5, 6, 7, 8, 9, 10, 11, or 12 days after the beginning of step (i);
(b) the percentage of naïve cells, for example, naïve T cells, for example, CD45RA+ CD45RO− CCR7+ T cells, in the population of cells from step (iii) is higher (for example, at least 1.2, 1.4, 1.6, 1.8, 2.0, 2.2, 2.4, 2.6, 2.8, or 3-fold higher) than the percentage of naïve cells, for example, naïve T cells, for example, CD45RA+ CD45RO− CCR7+ T cells, in cells made by an otherwise similar method in which step (iii) is performed more than 26 hours after the beginning of step (i), for example, more than 5, 6, 7, 8, 9, 10, 11, or 12 days after the beginning of step (i);
(c) the percentage of CAR-expressing naïve T cells, for example, CAR-expressing CD45RA+ CD45RO− CCR7+ T cells, in the population of cells from step (iii) is higher (for example, at least 4, 6, 8, 10, or 12-fold higher) than the percentage of CAR-expressing naïve T cells, for example, CAR-expressing CD45RA+CD45RO− CCR7+ T cells, in cells made by an otherwise similar method in which step (iii) is performed more than 26 hours after the beginning of step (i), for example, more than 5, 6, 7, 8, 9, 10, 11, or 12 days after the beginning of step (i);
(d) the population of cells from step (iii) shows a higher percentage of naïve cells, for example, naïve T cells, for example, CD45RA+CD45RO− CCR7+ T cells (for example, at least 10, 20, 30, or 40% higher), compared with cells made by an otherwise similar method which further comprises, after step (ii) and prior to step (iii), expanding the population of cells (for example, T cells) in vitro for more than 3 days, for example, for 5, 6, 7, 8 or 9 days;
(e) the percentage of naïve cells, for example, naïve T cells, for example, CD45RA+CD45RO− CCR7+ T cells, in the population of cells from step (iii) is higher (for example, at least 1.2, 1.4, 1.6, 1.8, 2.0, 2.2, 2.4, 2.6, 2.8, or 3-fold higher) than the percentage of naïve cells, for example, naïve T cells, for example, CD45RA+CD45RO− CCR7+ T cells, in cells made by an otherwise similar method which further comprises, after step (ii) and prior to step (iii), expanding the population of cells (for example, T cells) in vitro for more than 3 days, for example, for 5, 6, 7, 8 or 9 days; or
(f) the percentage of CAR-expressing naïve T cells, for example, CAR-expressing CD45RA+CD45RO− CCR7+ T cells, in the population of cells from step (iii) is higher (for example, at least 4, 6, 8, 10, or 12-fold higher) than the percentage of CAR-expressing naïve T cells, for example, CAR-expressing CD45RA+CD45RO− CCR7+ T cells, in cells made by an otherwise similar method which further comprises, after step (ii) and prior to step (iii), expanding the population of cells (for example, T cells) in vitro for more than 3 days, for example, for 5, 6, 7, 8 or 9 days.

24. The method of any one of claims 1-23, wherein:
(a) the percentage of central memory cells, for example, central memory T cells, for example, CD95+ central memory T cells, in the population of cells from step (iii) is the same as or differs by no more than 5 or 10% from the percentage of central memory cells, for example, central memory T cells, for example, CD95+ central memory T cells, in the population of cells at the beginning of step (i);
(b) the percentage of central memory cells, for example, central memory T cells, for example, CCR7+CD45RO+ T cells, in the population of cells from step (iii) is reduced by at least 20, 25, 30, 35, 40, 45, or 50%, as compared to the percentage of central memory cells, for example, central memory T cells, for example, CCR7+CD45RO+ T cells, in the population of cells at the beginning of step (i);
(c) the percentage of CAR-expressing central memory T cells, for example, CAR-expressing CCR7+CD45RO+ cells, decreases during the duration of step (ii), for example, decreases by, for example, at least 8, 10, 12, 14, 16, 18, or 20%, between 18-24 hours after the beginning of step (ii); or
(d) the percentage of central memory cells, for example, central memory T cells, for example, CCR7+CD45RO+ T cells, in the population of cells from step (iii) does not increase, or increases by no more than 5 or 10%, as compared to the percentage of central memory cells, for example, central memory T cells, for example, CCR7+CD45RO+ T cells, in the population of cells at the beginning of step (i).

25. The method of any one of claims 1-24, wherein:
(a) the population of cells from step (iii) shows a lower percentage of central memory cells, for example, central memory T cells, for example, CD95+ central memory T cells (for example, at least 10, 20, 30, or 40% lower), compared with cells made by an otherwise similar method in which step (iii) is performed more than 26 hours after the beginning of step (i), for example, more than 5, 6, 7, 8, 9, 10, 11, or 12 days after the beginning of step (i);
(b) the percentage of central memory cells, for example, central memory T cells, for example, CCR7+CD45RO+ T cells in the population of cells from step (iii) is lower (for example, at least 20, 30, 40, or 50% lower) than the percentage of central memory cells, for example, central memory T cells, for example, CCR7+CD45RO+ T cells, in cells made by an otherwise similar method in which step (iii) is performed more than 26 hours after the beginning of step (i), for example, more than 5, 6, 7, 8, 9, 10, 11, or 12 days after the beginning of step (i);
(c) the percentage of CAR-expressing central memory T cells, for example, CAR-expressing CCR7+CD45RO+ T cells in the population of cells from step (iii) is lower (for example, at least 10, 20, 30, or 40% lower) than the percentage of CAR-expressing central memory T cells, for example, CAR-expressing CCR7+CD45RO+ T cells, in cells made by an otherwise similar method in which step (iii) is performed more than 26 hours after the beginning of step (i), for example, more than 5, 6, 7, 8, 9, 10, 11, or 12 days after the beginning of step (i);
(d) the population of cells from step (iii) shows a lower percentage of central memory cells, for example, central memory T cells, for example, CD95+ central memory T cells (for example, at least 10, 20, 30, or 40% lower), compared with cells made by an otherwise similar method which further comprises, after step (ii) and prior to step (iii), expanding the population of cells (for example, T cells) in vitro for more than 3 days, for example, for 5, 6, 7, 8 or 9 days;
(e) the percentage of central memory cells, for example, central memory T cells, for example, CCR7+CD45RO+ T cells in the population of cells from step (iii) is lower (for example, at least 20, 30, 40, or 50% lower) than the percentage of central memory cells, for example, central memory T cells, for example, CCR7+CD45RO+ T cells, in cells made by an otherwise similar method which further comprises, after step (ii) and prior to step (iii), expanding the population of cells (for example, T cells) in vitro for more than 3 days, for example, for 5, 6, 7, 8 or 9 days; or
(f) the percentage of CAR-expressing central memory T cells, for example, CAR-expressing CCR7+CD45RO+ T cells in the population of cells from step (iii) is lower (for example, at least 10, 20, 30, or 40% lower) than the percentage of CAR-expressing central memory T cells, for example, CAR-expressing CCR7+CD45RO+ T cells, in cells made by an otherwise similar method which further comprises, after step (ii) and prior to step (iii), expanding the population of cells (for example, T cells) in vitro for more than 3 days, for example, for 5, 6, 7, 8 or 9 days.

26. The method of any one of claims 1-25, wherein:
(a) the percentage of stem memory T cells, for example, CD45RA+CD95+IL-2 receptor β+CCR7+CD62L+ T cells, in the population of cells from step (iii) is increased, as compared to the percentage of stem memory T cells, for example, CD45RA+CD95+IL-2 receptor β+CCR7+CD62L+ T cells, in the population of cells at the beginning of step (i);
(b) the percentage of CAR-expressing stem memory T cells, for example, CAR-expressing CD45RA+CD95+IL-2 receptor β+CCR7+CD62L+ T cells, in the population of cells from step (iii) is increased, as compared to the percentage of CAR-expressing stem memory T cells, for example, CAR-expressing CD45RA+CD95+IL-2 receptor β+CCR7+CD62L+ T cells, in the population of cells at the beginning of step (i);
(c) the percentage of stem memory T cells, for example, CD45RA+CD95+IL-2 receptor β+CCR7+CD62L+ T cells, in the population of cells from step (iii) is higher than the percentage of stem memory T cells, for example, CD45RA+CD95+IL-2 receptor β+CCR7+CD62L+ T cells, in cells made by an otherwise similar method in which step (iii) is performed more than 26 hours after the beginning of step (i), for example, more than 5, 6, 7, 8, 9, 10, 11, or 12 days after the beginning of step (i); or
(d) the percentage of CAR-expressing stem memory T cells, for example, CAR-expressing CD45RA+CD95+IL-2 receptor β+CCR7+CD62L+ T cells, in the population of cells from step (iii) is higher than the percentage of CAR-expressing stem memory T cells, for example, CAR-expressing CD45RA+CD95+IL-2 receptor β+CCR7+CD62L+ T cells, in cells made by an otherwise similar method in which step (iii) is performed more than 26 hours after the beginning of step (i), for example, more than 5, 6, 7, 8, 9, 10, 11, or 12 days after the beginning of step (i);
(e) the percentage of stem memory T cells, for example, CD45RA+CD95+IL-2 receptor β+CCR7+CD62L+ T cells, in the population of cells from step (iii) is higher than the percentage of stem memory T cells, for example, CD45RA+CD95+IL-2 receptor β+CCR7+CD62L+ T cells, in cells made by an otherwise similar method which further comprises, after step (ii) and prior to step (iii), expanding the population of cells (for example, T cells) in vitro for more than 3 days, for example, for 5, 6, 7, 8 or 9 days; or
(f) the percentage of CAR-expressing stem memory T cells, for example, CAR-expressing CD45RA+CD95+IL-2 receptor β+CCR7+CD62L+ T cells, in the population of cells from step (iii) is higher than the percentage of CAR-expressing stem memory T cells, for example, CAR-expressing CD45RA+CD95+IL-2 receptor β+CCR7+CD62L+ T cells, in cells made by an otherwise similar method which further comprises, after step (ii) and prior to step (iii), expanding the population of cells (for example, T cells) in vitro for more than 3 days, for example, for 5, 6, 7, 8 or 9 days.

27. The method of any one of claims 1-26, wherein:
(a) the median GeneSetScore (Up TEM vs. Down TSCM) of the population of cells from step (iii) is about the same as or differs by no more than (for example, increased by no more than) about 25, 50, 75, 100, or 125% from the median GeneSetScore (Up TEM vs. Down TSCM) of the population of cells at the beginning of step (i);
(b) the median GeneSetScore (Up TEM vs. Down TSCM) of the population of cells from step (iii) is lower (for example, at least about 100, 150, 200, 250, or 300% lower) than the median GeneSetScore (Up TEM vs. Down TSCM) of:
cells made by an otherwise similar method in which step (iii) is performed more than 26 hours after the beginning of step (i), for example, more than 5, 6, 7, 8, 9, 10, 11, or 12 days after the beginning of step (i), or
cells made by an otherwise similar method which further comprises, after step (ii) and prior to step (iii), expanding the population of cells (for example, T cells) in vitro for more than 3 days, for example, for 5, 6, 7, 8 or 9 days;
(c) the median GeneSetScore (Up Treg vs. Down Teff) of the population of cells from step (iii) is about the same as or differs by no more than (for example, increased by no more than) about 25, 50, 100, 150, or 200% from the median GeneSetScore (Up Treg vs. Down Teff) of the population of cells at the beginning of step (i);
(d) the median GeneSetScore (Up Treg vs. Down Teff) of the population of cells from step (iii) is lower (for example, at least about 50, 100, 125, 150, or 175% lower) than the median GeneSetScore (Up Treg vs. Down Teff) of:
cells made by an otherwise similar method in which step (iii) is performed more than 26 hours after the beginning of step (i), for example, more than 5, 6, 7, 8, 9, 10, 11, or 12 days after the beginning of step (i), or
cells made by an otherwise similar method which further comprises, after step (ii) and prior to step (iii), expanding the population of cells (for example, T cells) in vitro for more than 3 days, for example, for 5, 6, 7, 8 or 9 days;
(e) the median GeneSetScore (Down stemness) of the population of cells from step (iii) is about the same as or differs by no more than (for example, increased by no more than) about 25, 50, 100, 150, 200, or 250% from the median GeneSetScore (Down stemness) of the population of cells at the beginning of step (i);
(f) the median GeneSetScore (Down stemness) of the population of cells from step (iii) is lower (for example, at least about 50, 100, or 125% lower) than the median GeneSetScore (Down stemness) of:
cells made by an otherwise similar method in which step (iii) is performed more than 26 hours after the beginning of step (i), for example, more than 5, 6, 7, 8, 9, 10, 11, or 12 days after the beginning of step (i), or
cells made by an otherwise similar method which further comprises, after step (ii) and prior to step (iii), expanding the population of cells (for example, T cells) in vitro for more than 3 days, for example, for 5, 6, 7, 8 or 9 days;
(g) the median GeneSetScore (Up hypoxia) of the population of cells from step (iii) is about the same as or differs by no more than (for example, increased by no more than) about 125, 150, 175, or 200% from the median GeneSetScore (Up hypoxia) of the population of cells at the beginning of step (i);
(h) the median GeneSetScore (Up hypoxia) of the population of cells from step (iii) is lower (for example, at least about 40, 50, 60, 70, or 80% lower) than the median GeneSetScore (Up hypoxia) of:
cells made by an otherwise similar method in which step (iii) is performed more than 26 hours after the beginning of step (i), for example, more than 5, 6, 7, 8, 9, 10, 11, or 12 days after the beginning of step (i), or
cells made by an otherwise similar method which further comprises, after step (ii) and prior to step (iii), expanding the population of cells (for example, T cells) in vitro for more than 3 days, for example, for 5, 6, 7, 8 or 9 days;
(j) the median GeneSetScore (Up autophagy) of the population of cells from step (iii) is about the same as or differs by no more than (for example, increased by no more than) about 180, 190, 200, or 210% from the median GeneSetScore (Up autophagy) of the population of cells at the beginning of step (i); or
(k) the median GeneSetScore (Up autophagy) of the population of cells from step (iii) is lower (for example, at least 20, 30, or 40% lower) than the median GeneSetScore (Up autophagy) of:
cells made by an otherwise similar method in which step (iii) is performed more than 26 hours after the beginning of step (i), for example, more than 5, 6, 7, 8, 9, 10, 11, or 12 days after the beginning of step (i), or
cells made by an otherwise similar method which further comprises, after step (ii) and prior to step (iii), expanding the population of cells (for example, T cells) in vitro for more than 3 days, for example, for 5, 6, 7, 8 or 9 days.

28. The method of any one of claims 1-27, wherein the population of cells from step (iii), after being incubated with a cell expressing an antigen recognized by the CAR, secretes IL-2 at a higher level (for example, at least 2, 4, 6, 8, 10, 12, or 14-fold higher) than cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days, for example, as assessed using methods described in Example 8 with respect to FIGS. 29C-29D.

29. The method of any one of claims 1-28, wherein the population of cells from step (iii), after being administered in vivo, persists longer or expands at a higher level (for example, as assessed using methods described in Example 1 with respect to FIG. 4C), compared with cells made by an otherwise similar method in which step (iii) is performed more than 26 hours after the beginning of step (i), for example, more than 5, 6, 7, 8, 9, 10, 11, or 12 days after the beginning of step (i), or compared with cells made by an otherwise similar method which further comprises, after step (ii) and prior to step (iii), expanding the population of cells (for example, T cells) in vitro for more than 3 days, for example, for 5, 6, 7, 8 or 9 days.

30. The method of any one of claims 1-29, wherein the population of cells from step (iii), after being administered in vivo, shows a stronger anti-tumor activity (for example, a stronger anti-tumor activity at a low dose, for example, a dose no more than 0.15×10⁶, 0.2×10⁶, 0.25×10⁶, or 0.3×10⁶ viable CAR-expressing cells) than cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days.

31. The method of any one of claims 1-30, the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i), optionally wherein the number of living cells in the population of cells from step (iii) decreases from the number of living cells in the population of cells at the beginning of step (i).

32. The method of any one of claims 1-31, wherein the population of cells from step (iii) are not expanded, or expanded by less than 2 hours, for example, less than 1 or 1.5 hours, compared to the population of cells at the beginning of step (i).

33. The method of any one of claims 1-32, wherein steps (i) and/or (ii) are performed in cell media (for example, serum-free media) comprising IL-2, IL-15 (for example, hetIL-15 (IL15/sIL-15Ra)), IL-7, IL-21, IL-6 (for example, IL-6/sIL-6Ra), a LSD1 inhibitor, a MALT1 inhibitor, or a combination thereof.

34. The method of any one of claims 1-33, wherein steps (i) and/or (ii) are performed in serum-free cell media comprising a serum replacement.

35. The method of claim 30, wherein the serum replacement is CTS™ Immune Cell Serum Replacement (ICSR).

36. The method of any one of claims 1-35, further comprising prior to step (i):
(iv) (optionally) receiving a fresh leukapheresis product (or an alternative source of hematopoietic tissue such as a fresh whole blood product, a fresh bone marrow product, or a fresh tumor or organ biopsy or removal (for example, a fresh product from thymectomy)) from an entity, for example, a laboratory, hospital, or healthcare provider, and
(v) isolating the population of cells (for example, T cells, for example, CD8+ and/or CD4+ T cells) contacted in step (i) from a fresh leukapheresis product (or an alternative source of hematopoietic tissue such as a fresh whole blood product, a fresh bone marrow product, or a fresh tumor or organ biopsy or removal (for example, a fresh product from thymectomy)), optionally wherein:
step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v), or
the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

37. The method of any one of claims 1-36, further comprising prior to step (i): receiving cryopreserved T cells isolated from a leukapheresis product (or an alternative source of hematopoietic tissue such as cryopreserved T cells isolated from whole blood, bone marrow, or tumor or organ biopsy or removal (for example, thymectomy)) from an entity, for example, a laboratory, hospital, or healthcare provider.

38. The method of any one of claims 1-36, further comprising prior to step (i):
(iv) (optionally) receiving a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)) from an entity, for example, a laboratory, hospital, or healthcare provider, and
(v) isolating the population of cells (for example, T cells, for example, CD8+ and/or CD4+ T cells) contacted in step (i) from a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)), optionally wherein:
step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v), or
the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

39. The method of any one of claims 1-38, further comprising step (vi):
culturing a portion of the population of cells from step (iii) for at least 2, 2.5, 3, 3.5, 4, 4.5, 5, 5.5, 6, 6.5, or 7 days, for example, at least 2 days and no more than 7 days, and measuring CAR expression level in the portion (for example, measuring the percentage of viable, CAR-expressing cells in the portion), optionally wherein:
step (iii) comprises harvesting and freezing the population of cells (for example, T cells) and step (vi) comprises thawing a portion of the population of cells from step (iii), culturing the portion for at least 2, 2.5, 3, 3.5, 4, 4.5, 5, 5.5, 6, 6.5, or 7 days, for example, at least 2 days and no more than 7 days, and measuring CAR expression level in the portion (for example, measuring the percentage of viable, CAR-expressing cells in the portion).

40. The method of any one of claims 1-39, wherein step (ii) further comprises adding F10⁸ during transduction and/or contacting the population of cells (for example, T cells) with an shRNA that targets Tet2.

41. The method of any one of claims 1-40, wherein the population of cells at the beginning of step (i) or step (1) has been enriched for IL6R-expressing cells (for example, cells that are positive for IL6Rα and/or IL6Rβ).

42. The method of any one of claims 1-41, wherein the population of cells at the beginning of step (i) or step (1) comprises no less than 50, 60, or 70% of IL6R-expressing cells (for example, cells that are positive for IL6Rα and/or IL6Rβ).

43. The method of any one of claims 1-42, wherein steps (i) and (ii) or steps (1) and (2) are performed in cell media comprising IL-15 (for example, hetIL-15 (IL15/sIL-15Ra)).

44. The method of claim 43, wherein IL-15 increases the ability of the population of cells to expand, for example, 10, 15, 20, or 25 days later.

45. The method of claim 43, wherein IL-15 increases the percentage of IL6Rβ-expressing cells in the population of cells.

46. The method of any one of claims 1-45, wherein the CAR comprises an antigen binding domain, a transmembrane domain, and an intracellular signaling domain.

47. The method of claim 46, wherein the antigen binding domain binds to an antigen chosen from: CD19, CD20, CD22, BCMA, mesothelin, EGFRvIII, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (for example, ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRα4, or a peptide of any of these antigens presented on MHC.

48. The method of claim 46 or 47, wherein the antigen binding domain comprises a CDR, VH, VL, scFv or CAR sequence disclosed herein, optionally wherein:
(a) the antigen binding domain binds to BCMA and comprises a CDR, VH, VL, scFv or CAR sequence disclosed in Tables 3-15, or a sequence having at least 80%, 85%, 90%, 95%, or 99% identity thereto;
(b) the antigen binding domain binds to CD19 and comprises a CDR, VH, VL, scFv or CAR sequence disclosed in Table 2, or a sequence having at least 80%, 85%, 90%, 95%, or 99% identity thereto;
(c) the antigen binding domain binds to CD20 and comprises a CDR, VH, VL, scFv or CAR sequence disclosed herein, or a sequence having at least 80%, 85%, 90%, 95%, or 99% identity thereto; or
(d) the antigen binding domain binds to CD22 and comprises a CDR, VH, VL, scFv or CAR sequence disclosed herein, or a sequence having at least 80%, 85%, 90%, 95%, or 99% identity thereto.

49. The method of any one of claims 46-48, wherein the antigen binding domain comprises a VH and a VL, wherein the VH and VL are connected by a linker, optionally wherein the linker comprises the amino acid sequence of SEQ ID NO: 63 or 104.

50. The method of any one of claims 46-49, wherein:
(a) the transmembrane domain comprises a transmembrane domain of a protein chosen from the alpha, beta or zeta chain of T-cell receptor, CD28, CD3 epsilon, CD45, CD4, CD5, CD8, CD9, CD16, CD22, CD33, CD37, CD64, CD80, CD86, CD134, CD137 and CD154,
(b) the transmembrane domain comprises a transmembrane domain of CD8,
(c) the transmembrane domain comprises the amino acid sequence of SEQ ID NO: 6, or an amino acid sequence having at least about 85%, 90%, 95%, or 99% sequence identity thereof, or
(d) the nucleic acid molecule comprises a nucleic acid sequence encoding the transmembrane domain, wherein the nucleic acid sequence comprises the nucleic acid sequence of SEQ ID NO: 17, or a nucleic acid sequence having at least about 85%, 90%, 95%, or 99% sequence identity thereof.

51. The method of any one of claims 46-50, wherein the antigen binding domain is connected to the transmembrane domain by a hinge region, optionally wherein:
(a) the hinge region comprises the amino acid sequence of SEQ ID NO: 2, 3, or 4, or an amino acid sequence having at least about 85%, 90%, 95%, or 99% sequence identity thereof, or
(b) the nucleic acid molecule comprises a nucleic acid sequence encoding the hinge region, wherein the nucleic acid sequence comprises the nucleic acid sequence of SEQ ID NO:
13, 14, or 15, or a nucleic acid sequence having at least about 85%, 90%, 95%, or 99% sequence identity thereof.

52. The method of any one of claims 46-51, wherein the intracellular signaling domain comprises a primary signaling domain, optionally wherein the primary signaling domain comprises a functional signaling domain derived from CD3 zeta, TCR zeta, FcR gamma, FcR beta, CD3 gamma, CD3 delta, CD3 epsilon, CD5, CD22, CD79a, CD79b, CD278 (ICOS), FcεRI, DAP10, DAP12, or CD66d, optionally wherein:
(a) the primary signaling domain comprises a functional signaling domain derived from CD3 zeta,
(b) the primary signaling domain comprises the amino acid sequence of SEQ ID NO: 9 or 10, or an amino acid sequence having at least about 85%, 90%, 95%, or 99% sequence identity thereof, or
(c) the nucleic acid molecule comprises a nucleic acid sequence encoding the primary signaling domain, wherein the nucleic acid sequence comprises the nucleic acid sequence of SEQ ID NO: 20 or 21, or a nucleic acid sequence having at least about 85%, 90%, 95%, or 99% sequence identity thereof.

53. The method of any one of claims 46-52, wherein the intracellular signaling domain comprises a costimulatory signaling domain, optionally wherein the costimulatory signaling domain comprises a functional signaling domain derived from a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly10⁸), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, or a ligand that specifically binds with CD83, optionally wherein:
(a) the costimulatory signaling domain comprises a functional signaling domain derived from 4-1BB,
(b) the costimulatory signaling domain comprises the amino acid sequence of SEQ ID NO: 7, or an amino acid sequence having at least about 85%, 90%, 95%, or 99% sequence identity thereof, or
(c) the nucleic acid molecule comprises a nucleic acid sequence encoding the costimulatory signaling domain, wherein the nucleic acid sequence comprises the nucleic acid sequence of SEQ ID NO: 18, or a nucleic acid sequence having at least about 85%, 90%, 95%, or 99% sequence identity thereof.

54. The method of any one of claims 46-53, wherein the intracellular signaling domain comprises a functional signaling domain derived from 4-1BB and a functional signaling domain derived from CD3 zeta, optionally wherein the intracellular signaling domain comprises the amino acid sequence of SEQ ID NO: 7 (or an amino acid sequence having at least about 85%, 90%, 95%, or 99% sequence identity thereof) and the amino acid sequence of SEQ ID NO: 9 or 10 (or an amino acid sequence having at least about 85%, 90%, 95%, or 99% sequence identity thereof), optionally wherein the intracellular signaling domain comprises the amino acid sequence of SEQ ID NO: 7 and the amino acid sequence of SEQ ID NO: 9 or 10.

55. The method of any one of claims 46-54, wherein the CAR further comprises a leader sequence comprising the amino acid sequence of SEQ ID NO: 1.

56. A population of CAR-expressing cells (for example, autologous or allogeneic CAR-expressing T cells or NK cells) made by the method of any one of claims 1-55.

57. A pharmaceutical composition comprising the population of CAR-expressing cells of claim 56 and a pharmaceutically acceptable carrier.

58. A method of increasing an immune response in a subject, comprising administering the population of CAR-expressing cells of claim 56 or the pharmaceutical composition of claim 57 to the subject, thereby increasing an immune response in the subject.

59. A method of treating a cancer in a subject, comprising administering the population of CAR-expressing cells of claim 56 or the pharmaceutical composition of claim 57 to the subject, thereby treating the cancer in the subject.

60. The method of claim 59, wherein the cancer is a solid cancer, for example, chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof.

61. The method of claim 59, wherein the cancer is a liquid cancer, for example, chosen from: chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+large B-cell lymphoma, large B-cell lymphoma arising in HHV8-associated multicentric Castleman disease, primary effusion lymphoma, B-cell lymphoma, acute myeloid leukemia (AML), or unclassifiable lymphoma.

62. The method of any one of claims 58-61, further comprising administering a second therapeutic agent to the subject.

63. The method of any one of claims 58-62, wherein the population of CAR-expressing cells is administered at a dose determined based on the percentage of CAR-expressing cells measured in claim 39.

64. The population of CAR-expressing cells of claim 56 or the pharmaceutical composition of claim 57 for use in a method of increasing an immune response in a subject, said method comprising administering to the subject an effective amount of the population of CAR-expressing cells or an effective amount of the pharmaceutical composition.

65. The population of CAR-expressing cells of claim 56 or the pharmaceutical composition of claim 57 for use in a method of treating a cancer in a subject, said method comprising administering to the subject an effective amount of the population of CAR-expressing cells or an effective amount of the pharmaceutical composition.

66. A multispecific binding molecule comprising:
(i) an anti-CD3 binding domain,
(ii) an anti-CD28 binding domain comprising a heavy chain variable region (VH) comprising a heavy chain complementarity determining region 1 (HCDR1), a HCDR2, and a HCDR3, and a light chain variable region (VL) comprising a light chain complementarity determining region 1 (LCDR1), a LCDR2, and a LCDR3 wherein:
(a) the HCDR1, HCDR2, HCDR3, LCDR1, LCDR2, and LCDR3 comprise the amino acid sequences of SEQ ID NOs: 538, 539, 540, 530, 531, and 532, respectively;
(b) the HCDR1, HCDR2, HCDR3, LCDR1, LCDR2, and LCDR3 comprise the amino acid sequences of SEQ ID NOs: 541, 539, 540, 530, 531, and 532, respectively;
(c) the HCDR1, HCDR2, HCDR3, LCDR1, LCDR2, and LCDR3 comprise the amino acid sequences of SEQ ID NOs: 542, 543, 540, 533, 534, and 535, respectively; or
(d) the HCDR1, HCDR2, HCDR3, LCDR1, LCDR2, and LCDR3 comprise the amino acid sequences of SEQ ID NOs: 544, 545, 546, 536, 534, and 532, respectively; and
(iii) an Fc region comprising:
a L234A, L235A, S267K, and P329A mutation (LALASKPA), numbered according to the EU numbering system;
a L234A, L235A, and P329G mutation (LALAPG), numbered according to the EU numbering system;
a G237A, D265A, P329A, and S267K mutation (GADAPASK), numbered according to the EU numbering system;
a L234A, L235A, and G237A mutation (LALGA), numbered according to the EU numbering system;
a D265A, P329A, and S267K mutation (DAPASK), numbered according to the EU numbering system;
a G237A, D265A, and P329A mutation (GADAPA), numbered according to the EU numbering system; or
a L234A, L235A, and P329A mutation (LALAPA), numbered according to the EU numbering system.

67. The multispecific binding molecule of claim 66, wherein the anti-CD28 binding domain comprises:
(i) a VH comprising the amino acid sequence of SEQ ID NO: 547 or 548, or a sequence with at least 95% sequence identity to SEQ ID NO: 547 or 548;
(ii) a VL comprising the amino acid sequence of SEQ ID NO: 537, or a sequence with at least 95% sequence identity thereto;
(iii) a VH comprising the amino acid sequence of SEQ ID NO: 547 or a sequence with at least 95% sequence identity thereto, and a VL comprising the amino acid sequence of SEQ ID NO: 537, or a sequence with at least 95% sequence identity thereto; or
(iv) a VH comprising the amino acid sequence of SEQ ID NO: 548 or a sequence with at least 95% sequence identity thereto, and a VL comprising the amino acid sequence of SEQ ID NO: 537, or a sequence with at least 95% sequence identity thereto.

68. The multispecific binding molecule of claim 66 or 67, which further comprises a light chain constant region chosen from the light chain constant regions of kappa or lambda.

69. The multispecific binding molecule of any one of claims 66-68, wherein the Fc region comprises a CH2, a CH3, or both a CH2 and CH3, optionally wherein the CH2 and/or CH3 are selected from IgG1, IgG2, IgG3, or IgG4.

70. The multispecific binding molecule of any one of claims 66-69, wherein the anti-CD3 binding domain comprises:
(i) a HCDR1, HCDR2, HCDR3, LCDR1, LCDR2, and LCDR3 of an anti-CD3 antibody molecule of Table 27 (for example the anti-CD3 (1), anti-CD3 (2), anti-CD3 (3), or anti-CD3 (4)); or
(ii) the amino acid sequence of any VH and/or VL region of an anti-CD3 antibody molecule provided in Table 27 (for example the anti-CD3 (1), anti-CD3 (2), anti-CD3 (3), or anti-CD3 (4)), or an amino acid sequence at least 95% identical thereto.

71. A multispecific binding molecule, comprising a first binding domain and a second binding domain, wherein the multispecific binding molecule comprises:
(i) a first polypeptide comprising from N-terminal to C-terminal: VH of the first binding domain, VL of the first binding domain, VH of the second binding domain, CH1 and an Fc region, which comprises a CH2 and a CH3; and
(ii) a second polypeptide comprising from N-terminal to C-terminal: VL of the second binding domain and CL;
wherein the Fc region comprises:
a L234A, L235A, S267K, and P329A mutation (LALASKPA), numbered according to the EU numbering system;
a L234A, L235A, and P329G mutation (LALAPG), numbered according to the EU numbering system;
a G237A, D265A, P329A, and S267K mutation (GADAPASK), numbered according to the EU numbering system;
a L234A, L235A, and G237A mutation (LALGA), numbered according to the EU numbering system;
a D265A, P329A, and S267K mutation (DAPASK), numbered according to the EU numbering system;
a G237A, D265A, and P329A mutation (GADAPA), numbered according to the EU numbering system; or
a L234A, L235A, and P329A mutation (LALAPA), numbered according to the EU numbering system.

72. A multispecific binding molecule, comprising a first binding domain and a second binding domain, wherein the multispecific binding molecule comprises:
(i) a first polypeptide comprising from N-terminal to C-terminal: VH of the second binding domain, CH1, an Fc region comprising a CH2 and a CH3, VH of the first binding domain, and VL of the first binding domain; and
(ii) a second polypeptide comprising from N-terminal to C-terminal: VL of the second binding domain and CL;
wherein the Fc region comprises:
a L234A, L235A, S267K, and P329A mutation (LALASKPA), numbered according to the EU numbering system;
a L234A, L235A, and P329G mutation (LALAPG), numbered according to the EU numbering system;
a G237A, D265A, P329A, and S267K mutation (GADAPASK), numbered according to the EU numbering system;
a L234A, L235A, and G237A mutation (LALGA), numbered according to the EU numbering system;
a D265A, P329A, and S267K mutation (DAPASK), numbered according to the EU numbering system;
a G237A, D265A, and P329A mutation (GADAPA), numbered according to the EU numbering system; or
a L234A, L235A, and P329A mutation (LALAPA), numbered according to the EU numbering system.

73. A multispecific binding molecule, comprising a first binding domain and a second binding domain, wherein the multispecific binding molecule comprises:
(i) a first polypeptide comprising from N-terminal to C-terminal: VH of the second binding domain, CH1, VH of the first binding domain, VL of the first binding domain, and an Fc region comprising a CH2 and a CH3; and
(ii) a second polypeptide comprising from N-terminal to C-terminal: VL of the second binding domain and CL;
wherein the Fc region comprises:
a L234A, L235A, S267K, and P329A mutation (LALASKPA), numbered according to the EU numbering system;
a L234A, L235A, and P329G mutation (LALAPG), numbered according to the EU numbering system;
a G237A, D265A, P329A, and S267K mutation (GADAPASK), numbered according to the EU numbering system;
a L234A, L235A, and G237A mutation (LALGA), numbered according to the EU numbering system;
a D265A, P329A, and S267K mutation (DAPASK), numbered according to the EU numbering system;
a G237A, D265A, and P329A mutation (GADAPA), numbered according to the EU numbering system; or
a L234A, L235A, and P329A mutation (LALAPA), numbered according to the EU numbering system.

74. The multispecific binding molecule of any one of claims 71-73, wherein the first binding domain comprises an anti-CD3 binding domain and the second binding domain comprises a costimulatory molecule binding domain.

75. The multispecific binding molecule of any one of claims 71-73, wherein the first binding domain comprises a costimulatory molecule binding domain and the second binding domain comprises an anti-CD3 binding domain.

76. The multispecific binding molecule of claim 74 or 75, wherein the costimulatory molecule binding domain comprises an anti-CD2 binding domain or an anti-CD28 binding domain.

77. The method of any one of claims 1-55, or the multispecific binding molecule of any one of claims 66-76, wherein the multispecific binding molecule comprises:
(i) a heavy chain comprising the amino acid sequence of any of SEQ ID NOs: 794, 795, 798, 800, or 815-817 or an amino acid sequence having at least 95% sequence identity thereto; and/or
(ii) a light chain comprising the amino acid sequence of any of SEQ ID NOs: 673, 796, 797, 799, or 801, or an amino acid sequence having at least 95% sequence identity thereto.

78. The method of any one of claims 1-55, or the multispecific binding molecule of any one of claims 66-77, wherein the multispecific binding molecule comprises:
(i) a heavy chain comprising the amino acid sequence of SEQ ID NO: 794, or an amino acid sequence having at least 95% sequence identity thereto, and a light chain comprising the amino acid sequence of SEQ ID NO: 796, or an amino acid sequence having at least 95% sequence identity thereto;
(ii) a heavy chain comprising the amino acid sequence of SEQ ID NO: 794, or an amino acid sequence having at least 95% sequence identity thereto, and a light chain comprising the amino acid sequence of SEQ ID NO: 797, or an amino acid sequence having at least 95% sequence identity thereto;
(iii) a heavy chain comprising the amino acid sequence of SEQ ID NO: 795, or an amino acid sequence having at least 95% sequence identity thereto, and a light chain comprising the amino acid sequence of SEQ ID NO: 796, or an amino acid sequence having at least 95% sequence identity thereto;
(iv) a heavy chain comprising the amino acid sequence of SEQ ID NO: 795, or an amino acid sequence having at least 95% sequence identity thereto, and a light chain comprising the amino acid sequence of SEQ ID NO: 797, or an amino acid sequence having at least 95% sequence identity thereto;
(v) a heavy chain comprising the amino acid sequence of SEQ ID NO: 798, or an amino acid sequence having at least 95% sequence identity thereto, and a light chain comprising the amino acid sequence of SEQ ID NO: 799, or an amino acid sequence having at least 95% sequence identity thereto;
(vi) a heavy chain comprising the amino acid sequence of SEQ ID NO: 815, or an amino acid sequence having at least 95% sequence identity thereto, and a light chain comprising the amino acid sequence of SEQ ID NO: 799, or an amino acid sequence having at least 95% sequence identity thereto;
(vii) a heavy chain comprising the amino acid sequence of SEQ ID NO: 800, or an amino acid sequence having at least 95% sequence identity thereto, and a light chain comprising the amino acid sequence of SEQ ID NO: 801, or an amino acid sequence having at least 95% sequence identity thereto;
(viii) a heavy chain comprising the amino acid sequence of SEQ ID NO: 816, or an amino acid sequence having at least 95% sequence identity thereto, and a light chain comprising the amino acid sequence of SEQ ID NO: 673, or an amino acid sequence having at least 95% sequence identity thereto; or
(ix) a heavy chain comprising the amino acid sequence of SEQ ID NO: 817, or an amino acid sequence having at least 95% sequence identity thereto, and a light chain comprising the amino acid sequence of SEQ ID NO: 673, or an amino acid sequence having at least 95% sequence identity thereto.

79. A method of activating cells (e.g., immune effector cells, e.g., T cells), comprising contacting (for example, binding) a population of cells (for example, T cells, for example, T cells isolated from a frozen or fresh leukapheresis product) with the multispecific binding molecule of any one of claims 66-78.

80. A method of transducing cells (e.g., immune effector cells, e.g., T cells), comprising contacting (for example, binding) a population of cells (for example, T cells, for example, T cells isolated from a frozen or fresh leukapheresis product) with (i) the multispecific binding molecule of any one of claims 66-78, and (ii) a nucleic acid molecule, e.g., a nucleic acid molecule encoding a CAR.


SEQUENCE LISTING (793 sequences)

SEQ ID NO: 1 - protein, 21 aa, synthetic construct
  REGION 1..21: note = source = /note="Description of Artificial Sequence: Syntheticpeptide"
MALPVTALLLPLALLLHAARP

SEQ ID NO: 2 - protein, 45 aa, synthetic construct
  REGION 1..45: note = source = /note="Description of Artificial Sequence: Syntheticpolypeptide"
TTTPAPRPPTPAPTIASQPLSLRPEACRPAAGGAVHTRGLDFACD

SEQ ID NO: 3 - protein, 230 aa, synthetic construct
  REGION 1..230: note = source = /note="Description of Artificial Sequence: Syntheticpolypeptide"
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 4 - protein, 282 aa, synthetic construct
  REGION 1..282: note = source = /note="Description of Artificial Sequence: Syntheticpolypeptide"
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 5 - protein, 10 aa, synthetic construct
  REGION 1..10: note = source = /note="Description of Artificial Sequence: Syntheticpeptide"
GGGGSGGGGS

SEQ ID NO: 6 - protein, 24 aa, synthetic construct
  REGION 1..24: note = source = /note="Description of Artificial Sequence: Syntheticpeptide"
IYIWAPLAGTCGVLLLSLVITLYC

SEQ ID NO: 7 - protein, 42 aa, synthetic construct
  REGION 1..42: note = source = /note="Description of Artificial Sequence: Syntheticpolypeptide"
KRGRKKLLYIFKQPFMRPVQTTQEEDGCSCRFPEEEEGGCEL

SEQ ID NO: 8 - protein, 48 aa, synthetic construct
  REGION 1..48: note = source = /note="Description of Artificial Sequence: Syntheticpolypeptide"
QRRKYRSNKGESPVEPAEPCRYSCPREEEGSTIPIQEDYRKPEPACSP

SEQ ID NO: 9 - protein, 112 aa, synthetic construct
  REGION 1..112: note = source = /note="Description of Artificial Sequence: Syntheticpolypeptide"
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 1184 bp, synthetic construct
  misc_feature 1..1184: note = source = /note="Description of Artificial Sequence: Syntheticpolynucleotide"
  source 1..1184: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 12 - DNA, 63 bp, synthetic construct
  misc_feature 1..63: note = source = /note="Description of Artificial Sequence: Syntheticoligonucleotide"
  source 1..63: mol_type = other DNA
atggccctgcctgtgacagccctgctgctgcctctggctctgctgctgcatgccgctagaccc

SEQ ID NO: 13 - DNA, 135 bp, synthetic construct
  misc_feature 1..135: note = source = /note="Description of Artificial Sequence: Syntheticpolynucleotide"
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 14 - DNA, 690 bp, synthetic construct
  misc_feature 1..690: note = source = /note="Description of Artificial Sequence: Syntheticpolynucleotide"
  source 1..690: mol_type = other DNA
gagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatg

SEQ ID NO: 15 - DNA, 847 bp, synthetic construct
  misc_feature 1..847: note = source = /note="Description of Artificial Sequence: Syntheticpolynucleotide"
  source 1..847: mol_type = other DNA
aggtggcccgaaagtcccaaggcccaggcatctagtgttcctactgcacagccccaggcagaaggcagcctagccaaagctactactgcacctgccactacgcgcaatactggccgtggcggggaggagaagaaaaaggagaaagagaaagaagaacaggaagagagggagaccaagacccctgaatgtccatcccatacccagccgctgggcgtctatctcttgactcccgcagtacaggacttgtggcttagagataaggccacctttacatgtttcgtcgtgggctctgacctgaaggatgcccatttgacttgggaggttgccggaaaggtacccacagggggggttgaggaagggttgctggagcgccattccaatggctctcagagccagcactcaagactcacccttccgagatccctgtggaacgccgggacctctgtcacatgtactctaaatcatcctagcctgcccccacagcgtctgatggcccttagagagccagccgcccaggcaccagttaagcttagcctgaatctgctcgccagtagtgatcccccagaggccgccagctggctcttatgcgaagtgtccggctttagcccgcccaacatcttgctcatgtggctggaggaccagcgagaagtgaacaccagcggcttcgctccagcccggcccccaccccagccgggttctaccacattctgggcctggagtgtcttaagggtcccagcaccacctagcccccagccagccacatacacctgtgttgtgtcccatgaagatagcaggaccctgctaaatgcttctaggagtctggaggtttcctacgtgactgaccatt

SEQ ID NO: 16 - DNA, 30 bp, synthetic construct
  misc_feature 1..30: note = source = /note="Description of Artificial Sequence: Syntheticoligonucleotide"
  source 1..30: mol_type = other DNA
ggtggcggaggttctggaggtggaggttcc

SEQ ID NO: 17 - DNA, 72 bp, synthetic construct
  misc_feature 1..72: note = source = /note="Description of Artificial Sequence: Syntheticoligonucleotide"
  source 1..72: mol_type = other DNA
atctacatctgggcgcccttggccgggacttgtggggtccttctcctgtcactggttatcaccctttactgc

SEQ ID NO: 18 - DNA, 126 bp, synthetic construct
  misc_feature 1..126: note = source = /note="Description of Artificial Sequence: Syntheticpolynucleotide"
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 19 - DNA, 123 bp, synthetic construct
  misc_feature 1..123: note = source = /note="Description of Artificial Sequence: Syntheticpolynucleotide"
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 20 - DNA, 336 bp, synthetic construct
  misc_feature 1..336: note = source = /note="Description of Artificial Sequence: Syntheticpolynucleotide"
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 21 - DNA, 336 bp, Homo sapiens
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 22 - protein, 373 aa, synthetic construct
  REGION 1..373: note = source = /note="Description of Artificial Sequence: Syntheticpolypeptide"
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - DNA, 1182 bp, synthetic construct
  misc_feature 1..1182: note = source = /note="Description of Artificial Sequence: Syntheticpolynucleotide"
  source 1..1182: mol_type = other DNA
atggccctccctgtcactgccctgcttctccccctcgcactcctgctccacgccgctagaccacccggatggtttctggactctccggatcgcccgtggaatcccccaaccttctcaccggcactcttggttgtgactgagggcgataatgcgaccttcacgtgctcgttctccaacacctccgaatcattcgtgctgaactggtaccgcatgagcccgtcaaaccagaccgacaagctcgccgcgtttccggaagatcggtcgcaaccgggacaggattgtcggttccgcgtgactcaactgccgaatggcagagacttccacatgagcgtggtccgcgctaggcgaaacgactccgggacctacctgtgcggagccatctcgctggcgcctaaggcccaaatcaaagagagcttgagggccgaactgagagtgaccgagcgcagagctgaggtgccaactgcacatccatccccatcgcctcggcctgcggggcagtttcagaccctggtcacgaccactccggcgccgcgcccaccgactccggccccaactatcgcgagccagcccctgtcgctgaggccggaagcatgccgccctgccgccggaggtgctgtgcatacccggggattggacttcgcatgcgacatctacatttgggctcctctcgccggaacttgtggcgtgctccttctgtccctggtcatcaccctgtactgcaagcggggtcggaaaaagcttctgtacattttcaagcagcccttcatgaggcccgtgcaaaccacccaggaggaggacggttgctcctgccggttccccgaagaggaagaaggaggttgcgagctgcgcgtgaagttctcccggagcgccgacgcccccgcctataagcagggccagaaccagctgtacaacgaactgaacctgggacggcgggaagagtacgatgtgctggacaagcggcgcggccgggaccccgaaatgggcgggaagcctagaagaaagaaccctcaggaaggcctgtataacgagctgcagaaggacaagatggccgaggcctactccgaaattgggatgaagggagagcggcggaggggaaaggggcacgacggcctgtaccaaggactgtccaccgccaccaaggacacatacgatgccctgcacatgcaggcccttccccctcgc

SEQ ID NO: 24 - protein, 394 aa, synthetic construct
  REGION 1..394: note = source = /note="Description of Artificial Sequence: Syntheticpolypeptide"
MALPVTALLLPLALLLHAARPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 5 aa, synthetic construct
  REGION 1..5: note = source = /note="Description of Artificial Sequence: Syntheticpeptide"
  REGION 1..5: note = source = /note="See specification as filed for detailed description ofsubstitutions and preferred embodiments"
GGGGS

SEQ ID NO: 26 - protein, 30 aa, synthetic construct
  SITE 1..30: note = /note="This sequence may encompass 1-6 'Gly Gly Gly Gly Ser'repeating units"
  REGION 1..30: note = source = /note="Description of Artificial Sequence: Syntheticpolypeptide"
  REGION 1..30: note = source = /note="See specification as filed for detailed description ofsubstitutions and preferred embodiments"
GGGGSGGGGSGGGGSGGGGSGGGGSGGGGS

SEQ ID NO: 27 - protein, 20 aa, synthetic construct
  REGION 1..20: note = source = /note="Description of Artificial Sequence: Syntheticpeptide"
GGGGSGGGGSGGGGSGGGGS

SEQ ID NO: 28 - protein, 15 aa, synthetic construct
  REGION 1..15: note = source = /note="Description of Artificial Sequence: Syntheticpeptide"
GGGGSGGGGSGGGGS

SEQ ID NO: 29 - protein, 4 aa, synthetic construct
  REGION 1..4: note = source = /note="Description of Artificial Sequence: Syntheticpeptide"
GGGS

SEQ ID NO: 30 - RNA, 5000 nt, synthetic construct
  misc_feature 1..5000: note = /note="This sequence may encompass 50-5000 nucleotides"
  misc_feature 1..5000: note = source = /note="Description of Artificial Sequence: Syntheticpolynucleotide"
  source 1..5000: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 31 - DNA, 100 bp, synthetic construct
  misc_feature 1..100: note = source = /note="Description of Artificial Sequence: Syntheticpolynucleotide"
  source 1..100: mol_type = other DNA
tttttttttttttttttttttttttttttttttttttttttttttttttttttttttttttttttttttttttttttttttttttttttttttttttttt

SEQ ID NO: 32 - DNA, 5000 bp, synthetic construct
  misc_feature 1..5000: note = /note="This sequence may encompass 50-5000 nucleotides"
  misc_feature 1..5000: note = source = /note="Description of Artificial Sequence: Syntheticpolynucleotide"
  source 1..5000: mol_type = other DNA
tttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

SEQ ID NO: 33 - RNA, 5000 nt, synthetic construct; identical to SEQ ID NO: 30
  misc_feature 1..5000: note = /note="This sequence may encompass 100-5000 nucleotides"
  misc_feature 1..5000: note = source = /note="Description of Artificial Sequence: Syntheticpolynucleotide"
  source 1..5000: mol_type = other RNA

SEQ ID NO: 34 - RNA, 400 nt, synthetic construct
  misc_feature 1..400: note = /note="This sequence may encompass 100-400 nucleotides"
  misc_feature 1..400: note = source = /note="Description of Artificial Sequence: Syntheticpolynucleotide"
  misc_feature 1..400: note = source = /note="See specification as filed for detailed description ofsubstitutions and preferred embodiments"
  source 1..400: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 35 - RNA, 2000 nt, synthetic construct
  misc_feature 1..2000: note = /note="This sequence may encompass 50-2000 nucleotides"
  misc_feature 1..2000: note = source = /note="Description of Artificial Sequence: Syntheticpolynucleotide"
  source 1..2000: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 36 - protein, 41 aa, synthetic construct
  REGION 1..41: note = source = /note="Description of Artificial Sequence: Syntheticpolypeptide"
RSKRSRLLHSDYMNMTPRRPGPTRKHYQPYAPPRDFAAYRS

SEQ ID NO: 37 - DNA, 123 bp, synthetic construct; identical to SEQ ID NO: 19
  misc_feature 1..123: note = source = /note="Description of Artificial Sequence: Syntheticpolynucleotide"
  source 1..123: mol_type = other DNA

SEQ ID NO: 38 - protein, 35 aa, synthetic construct
  REGION 1..35: note = source = /note="Description of Artificial Sequence: Syntheticpolypeptide"
TKKKYSSSVHDPNGEYMFMRAVNTAKKSRLTDVTL

SEQ ID NO: 39 - DNA, 105 bp, synthetic construct
  misc_feature 1..105: note = source = /note="Description of Artificial Sequence: Syntheticpolynucleotide"
  source 1..105: mol_type = other DNA
acaaaaaagaagtattcatccagtgtgcacgaccctaacggtgaatacatgttcatgagagcagtgaacacagccaaaaaatccagactcacagatgtgacccta

SEQ ID NO: 40 - DNA, 30 bp, synthetic construct
  misc_feature 1..30: note = source = /note="Description of Artificial Sequence: Syntheticoligonucleotide"
  source 1..30: mol_type = other DNA
ggtggcggaggttctggaggtgggggttcc

SEQ ID NO: 41 - protein, 4 aa, synthetic construct
  REGION 1..4: note = source = /note="Description of Artificial Sequence: Syntheticpeptide"
  REGION 1..4: note = source = /note="See specification as filed for detailed description ofsubstitutions and preferred embodiments"
GGGS

SEQ ID NO: 42 - protein, 40 aa, synthetic construct
  SITE 1..40: note = /note="This sequence may encompass 1-10 'Gly Gly Gly Ser'repeating units"
  REGION 1..40: note = source = /note="Description of Artificial Sequence: Syntheticpolypeptide"
GGGSGGGSGGGSGGGSGGGSGGGSGGGSGGGSGGGSGGGS

SEQ ID NO: 43 - protein, 18 aa, synthetic construct
  REGION 1..18: note = source = /note="Description of Artificial Sequence: Syntheticpeptide"
GSTSGSGKPGSGEGSTKG

SEQ ID NO: 44 - protein, 5 aa, synthetic construct
  REGION 1..5: note = source = /note="Description of Artificial Sequence: Syntheticpeptide"
SYAMS

SEQ ID NO: 45 - protein, 17 aa, synthetic construct
  REGION 1..17: note = source = /note="Description of Artificial Sequence: Syntheticpeptide"
AISGSGGSTYYADSVKG

SEQ ID NO: 46 - protein, 14 aa, synthetic construct
  REGION 1..14: note = source = /note="Description of Artificial Sequence: Syntheticpeptide"
REWVPYDVSWYFDY

SEQ ID NO: 47 - protein, 7 aa, synthetic construct
  REGION 1..7: note = source = /note="Description of Artificial Sequence: Syntheticpeptide"
GFTFSSY

SEQ ID NO: 48 - protein, 6 aa, synthetic construct
  REGION 1..6: note = source = /note="Description of Artificial Sequence: Syntheticpeptide"
SGSGGS

SEQ ID NO: 49 - protein, 8 aa, synthetic construct
  REGION 1..8: note = source = /note="Description of Artificial Sequence: Syntheticpeptide"
GFTFSSYA

SEQ ID NO: 50 - protein, 8 aa, synthetic construct
  REGION 1..8: note = source = /note="Description of Artificial Sequence: Syntheticpeptide"
ISGSGGST

SEQ ID NO: 51 - protein, 16 aa, synthetic construct
  REGION 1..16: note = source = /note="Description of Artificial Sequence: Syntheticpeptide"
ARREWVPYDVSWYFDY

SEQ ID NO: 52 - protein, 123 aa, synthetic construct
  REGION 1..123: note = source = /note="Description of Artificial Sequence: Syntheticpolypeptide"
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWVPYDVSWYFDYWGQGTLVTVSS

SEQ ID NO: 53 - DNA, 369 bp, synthetic construct
  misc_feature 1..369: note = source = /note="Description of Artificial Sequence: Syntheticpolynucleotide"
  source 1..369: mol_type = other DNA
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtgggtgccctacgatgtcagctggtacttcgactactggggacagggcactctcgtgactgtgtcctcc

SEQ ID NO: 54 - protein, 11 aa, synthetic construct
  REGION 1..11: note = source = /note="Description of Artificial Sequence: Syntheticpeptide"
RASQSISSYLN

SEQ ID NO: 55 - protein, 7 aa, synthetic construct
  REGION 1..7: note = source = /note="Description of Artificial Sequence: Syntheticpeptide"
AASSLQS

SEQ ID NO: 56 - protein, 9 aa, synthetic construct
  REGION 1..9: note = source = /note="Description of Artificial Sequence: Syntheticpeptide"
QQSYSTPLT

SEQ ID NO: 57 - protein, 7 aa, synthetic construct
  REGION 1..7: note = source = /note="Description of Artificial Sequence: Syntheticpeptide"
SQSISSY

SEQ ID NO: 58 - skipped

SEQ ID NO: 59 - protein, 6 aa, synthetic construct
  REGION 1..6: note = source = /note="Description of Artificial Sequence: Syntheticpeptide"
SYSTPL

SEQ ID NO: 60 - protein, 6 aa, synthetic construct
  REGION 1..6: note = source = /note="Description of Artificial Sequence: Syntheticpeptide"
QSISSY

SEQ ID NO: 61 - protein, 107 aa, synthetic construct [sequence omitted]

SEQ ID NO: 62 - DNA, 321 bp, synthetic construct
  misc_feature 1..321: note = source = /note="Description of Artificial Sequence: Syntheticpolynucleotide"
  source 1..321: mol_type = other DNA
gacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaag

SEQ ID NO: 63 - protein, 20 aa, synthetic construct
  REGION 1..20: note = source = /note="Description of Artificial Sequence: Syntheticpeptide"
GGGGSGGGGSGGGGSGGGGS

SEQ ID NO: 64 - protein, 250 aa, synthetic construct
  REGION 1..250: note = source = /note="Description of Artificial Sequence: Syntheticpolypeptide"
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWVPYDVSWYFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIK

SEQ ID NO: 65 - DNA, 750 bp, synthetic construct
  misc_feature 1..750: note = source = /note="Description of Artificial Sequence: Syntheticpolynucleotide"
  source 1..750: mol_type = other DNA
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtgggtgccctacgatgtcagctggtacttcgactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaag

SEQ ID NO: 66 - protein, 473 aa, synthetic construct
  REGION 1..473: note = source = /note="Description of Artificial Sequence: Syntheticpolypeptide"
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWVPYDVSWYFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - DNA, 1419 bp, synthetic construct
  misc_feature 1..1419: note = source = /note="Description of Artificial Sequence: Syntheticpolynucleotide"
  source 1..1419: mol_type = other DNA
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtgggtgccctacgatgtcagctggtacttcgactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 68 - protein, 12 aa, synthetic construct
  REGION 1..12: note = source = /note="Description of Artificial Sequence: Syntheticpeptide"
REWWYDDWYLDY

SEQ ID NO: 69 - protein, 14 aa, synthetic construct
  REGION 1..14: note = source = /note="Description of Artificial Sequence: Syntheticpeptide"
ARREWWYDDWYLDY

SEQ ID NO: 70 - protein, 121 aa, synthetic construct
  REGION 1..121: note = source = /note="Description of Artificial Sequence: Syntheticpolypeptide"
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWYDDWYLDYWGQGTLVTVSS

SEQ ID NO: 71 - DNA, 363 bp, synthetic construct
  misc_feature 1..363: note = source = /note="Description of Artificial Sequence: Syntheticpolynucleotide"
  source 1..363: mol_type = other DNA
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggtacgacgattggtacctggactactggggacagggcactctcgtgactgtgtcctcc

SEQ ID NO: 72 - protein, 248 aa, synthetic construct
  REGION 1..248: note = source = /note="Description of Artificial Sequence: Syntheticpolypeptide"
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWYDDWYLDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIK

SEQ ID NO: 73 - DNA, 744 bp, synthetic construct
  misc_feature 1..744: note = source = /note="Description of Artificial Sequence: Syntheticpolynucleotide"
  source 1..744: mol_type = other DNA
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggtacgacgattggtacctggactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaag

SEQ ID NO: 74 - protein, 471 aa, synthetic construct
  REGION 1..471: note = source = /note="Description of Artificial Sequence: Syntheticpolypeptide"
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWYDDWYLDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - DNA, 1413 bp, synthetic construct
  misc_feature 1..1413: note = source = /note="Description of Artificial Sequence: Syntheticpolynucleotide"
  source 1..1413: mol_type = other DNA
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggtacgacgattggtacctggactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 76 - protein, 12 aa, synthetic construct
  REGION 1..12: note = source = /note="Description of Artificial Sequence: Syntheticpeptide"
REWWGESWLFDY

SEQ ID NO: 77 - protein, 14 aa, synthetic construct
  REGION 1..14: note = source = /note="Description of Artificial Sequence: Syntheticpeptide"
ARREWWGESWLFDY

SEQ ID NO: 78 - protein, 121 aa, synthetic construct
  REGION 1..121: note = source = /note="Description of Artificial Sequence: Syntheticpolypeptide"
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWGESWLFDYWGQGTLVTVSS

SEQ ID NO: 79 - DNA, 363 bp, synthetic construct
  misc_feature 1..363: note = source = /note="Description of Artificial Sequence: Syntheticpolynucleotide"
  source 1..363: mol_type = other DNA
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggggagaaagctggctgttcgactactggggacagggcactctcgtgactgtgtcctcc

SEQ ID NO: 80 - protein, 248 aa, synthetic construct
  REGION 1..248: note = source = /note="Description of Artificial Sequence: Syntheticpolypeptide"
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWGESWLFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIK

SEQ ID NO: 81 - DNA, 744 bp, synthetic construct
  misc_feature 1..744: note = source = /note="Description of Artificial Sequence: Syntheticpolynucleotide"
  source 1..744: mol_type = other DNA
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggggagaaagctggctgttcgactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaag

SEQ ID NO: 82 - protein, 471 aa, synthetic construct
  REGION 1..471: note = source = /note="Description of Artificial Sequence: Syntheticpolypeptide"
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWGESWLFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - DNA, 1413 bp, synthetic construct
  misc_feature 1..1413: note = source = /note="Description of Artificial Sequence: Syntheticpolynucleotide"
  source 1..1413: mol_type = other DNA
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggggagaaagctggctgttcgactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 84 - protein, 14 aa, synthetic construct
  VARIANT 4: note = /replace=" "
  VARIANT 5: note = /replace=" "
  VARIANT 6: note = /replace="Tyr"
  VARIANT 7: note = /replace="Tyr" or "Asp"
  VARIANT 8: note = /replace="Asp" or "Val"
  VARIANT 9: note = /replace="Asp"
  VARIANT 11: note = /replace="Tyr"
  VARIANT 12: note = /replace="Leu"
  SITE 1..14: note = /note="Variant residues given in the sequence have nopreference with respect to those in the annotationsfor variant positions"
  REGION 1..14: note = source = /note="Description of Artificial Sequence: Syntheticpeptide"
REWVPWGESWLFDY

SEQ ID NO: 85 - protein, 16 aa, synthetic construct
  VARIANT 6: note = /replace=" "
  VARIANT 7: note = /replace=" "
  VARIANT 8: note = /replace="Tyr"
  VARIANT 9: note = /replace="Tyr" or "Asp"
  VARIANT 10: note = /replace="Asp" or "Val"
  VARIANT 11: note = /replace="Asp"
  VARIANT 13: note = /replace="Tyr"
  VARIANT 14: note = /replace="Leu"
  SITE 1..16: note = /note="Variant residues given in the sequence have nopreference with respect to those in the annotationsfor variant positions"
  REGION 1..16: note = source = /note="Description of Artificial Sequence: Syntheticpeptide"
ARREWVPWGESWLFDY

SEQ ID NO: 86 - protein, 5 aa, synthetic construct
  REGION 1..5: note = source = /note="Description of Artificial Sequence: Syntheticpeptide"
SYGMH

SEQ ID NO: 87 - protein, 17 aa, synthetic construct
  REGION 1..17: note = source = /note="Description of Artificial Sequence: Syntheticpeptide"
VISYDGSNKYYADSVKG

SEQ ID NO: 88 - protein, 14 aa, synthetic construct
  REGION 1..14: note = source = /note="Description of Artificial Sequence: Syntheticpeptide"
SGYALHDDYYGLDV

SEQ ID NO: 89 - protein, 6 aa, synthetic construct
  REGION 1..6: note = source = /note="Description of Artificial Sequence: Syntheticpeptide"
SYDGSN

SEQ ID NO: 90 - protein, 8 aa, synthetic construct
  REGION 1..8: note = source = /note="Description of Artificial Sequence: Syntheticpeptide"
GFTFSSYG

SEQ ID NO: 91 - protein, 8 aa, synthetic construct
  REGION 1..8: note = source = /note="Description of Artificial Sequence: Syntheticpeptide"
ISYDGSNK

SEQ ID NO: 92 - protein, 16 aa, synthetic construct
  REGION 1..16: note = source = /note="Description of Artificial Sequence: Syntheticpeptide"
GGSGYALHDDYYGLDV

SEQ ID NO: 93 - protein, 123 aa, synthetic construct
  REGION 1..123: note = source = /note="Description of Artificial Sequence: Syntheticpolypeptide"
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSS

SEQ ID NO: 94 - DNA, 369 bp, synthetic construct
  misc_feature 1..369: note = source = /note="Description of Artificial Sequence: Syntheticpolynucleotide"
  source 1..369: mol_type = other DNA
caagtgcagctgcaggaatccggtggcggagtcgtgcagcctggaaggagcctgagactctcatgcgccgcgtcagggttcaccttttcctcctacgggatgcattgggtcagacaggcccccggaaagggactcgaatgggtggctgtgatcagctacgacggctccaacaagtactacgccgactccgtgaaaggccggttcactatctcccgggacaactccaagaacacgctgtatctgcaaatgaattcactgcgcgcggaggataccgctgtgtactactgcggtggctccggttacgccctgcacgatgactattacggccttgacgtctggggccagggaaccctcgtgactgtgtccagc

SEQ ID NO: 95 - protein, 14 aa, synthetic construct
  REGION 1..14: note = source = /note="Description of Artificial Sequence: Syntheticpeptide"
TGTSSDVGGYNYVS

SEQ ID NO: 96 - protein, 7 aa, synthetic construct
  REGION 1..7: note = source = /note="Description of Artificial Sequence: Syntheticpeptide"
DVSNRPS

SEQ ID NO: 97 - protein, 11 aa, synthetic construct
  REGION 1..11: note = source = /note="Description of Artificial Sequence: Syntheticpeptide"
SSYTSSSTLYV

SEQ ID NO: 98 - protein, 10 aa, synthetic construct
  REGION 1..10: note = source = /note="Description of Artificial Sequence: Syntheticpeptide"
TSSDVGGYNY

SEQ ID NO: 99 - skipped

SEQ ID NO: 100 - protein, 8 aa, synthetic construct
  REGION 1..8: note = source = /note="Description of Artificial Sequence: Syntheticpeptide"
YTSSSTLY

SEQ ID NO: 101 - protein, 9 aa, synthetic construct
  REGION 1..9: note = source = /note="Description of Artificial Sequence: Syntheticpeptide"
SSDVGGYNY

SEQ ID NO: 102 - protein, 111 aa, synthetic construct
  REGION 1..111: note = source = /note="Description of Artificial Sequence: Syntheticpolypeptide"
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVL

SEQ ID NO: 103 - DNA, 333 bp, synthetic construct
  misc_feature 1..333: note = source = /note="Description of Artificial Sequence: Syntheticpolynucleotide"
  source 1..333: mol_type = other DNA
cagagcgcactgactcagccggcatccgtgtccggtagccccggacagtcgattaccatctcctgtaccggcacctcctccgacgtgggagggtacaactacgtgtcgtggtaccagcagcacccaggaaaggcccctaagttgatgatctacgatgtgtcaaaccgcccgtctggagtctccaaccggttctccggctccaagtccggcaacaccgccagcctgaccattagcgggctgcaagccgaggatgaggccgactactactgctcgagctacacatcctcgagcaccctctacgtgttcggctcggggactaaggtcaccgtgctg

SEQ ID NO: 104 - protein, 15 aa, synthetic construct
  REGION 1..15: note = source = /note="Description of Artificial Sequence: Syntheticpeptide"
GGGGSGGGGSGGGGS

SEQ ID NO: 105 - protein, 249 aa, synthetic construct
  REGION 1..249: note = source = /note="Description of Artificial Sequence: Syntheticpolypeptide"
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVL

SEQ ID NO: 106 - DNA, 747 bp, synthetic construct
  misc_feature 1..747: note = source = /note="Description of Artificial Sequence: Syntheticpolynucleotide"
  source 1..747: mol_type = other DNA
caagtgcagctgcaggaatccggtggcggagtcgtgcagcctggaaggagcctgagactctcatgcgccgcgtcagggttcaccttttcctcctacgggatgcattgggtcagacaggcccccggaaagggactcgaatgggtggctgtgatcagctacgacggctccaacaagtactacgccgactccgtgaaaggccggttcactatctcccgggacaactccaagaacacgctgtatctgcaaatgaattcactgcgcgcggaggataccgctgtgtactactgcggtggctccggttacgccctgcacgatgactattacggccttgacgtctggggccagggaaccctcgtgactgtgtccagcggtggaggaggttcgggcggaggaggatcaggagggggtggatcgcagagcgcactgactcagccggcatccgtgtccggtagccccggacagtcgattaccatctcctgtaccggcacctcctccgacgtgggagggtacaactacgtgtcgtggtaccagcagcacccaggaaaggcccctaagttgatgatctacgatgtgtcaaaccgcccgtctggagtctccaaccggttctccggctccaagtccggcaacaccgccagcctgaccattagcgggctgcaagccgaggatgaggccgactactactgctcgagctacacatcctcgagcaccctctacgtgttcggctcggggactaaggtcaccgtgctg

SEQ ID NO: 107 - protein, 472 aa, synthetic construct
  REGION 1..472: note = source = /note="Description of Artificial Sequence: Syntheticpolypeptide"
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - DNA, 1416 bp, synthetic construct
  misc_feature 1..1416: note = source = /note="Description of Artificial Sequence: Syntheticpolynucleotide"
  source 1..1416: mol_type = other DNA
caagtgcagctgcaggaatccggtggcggagtcgtgcagcctggaaggagcctgagactctcatgcgccgcgtcagggttcaccttttcctcctacgggatgcattgggtcagacaggcccccggaaagggactcgaatgggtggctgtgatcagctacgacggctccaacaagtactacgccgactccgtgaaaggccggttcactatctcccgggacaactccaagaacacgctgtatctgcaaatgaattcactgcgcgcggaggataccgctgtgtactactgcggtggctccggttacgccctgcacgatgactattacggccttgacgtctggggccagggaaccctcgtgactgtgtccagcggtggaggaggttcgggcggaggaggatcaggagggggtggatcgcagagcgcactgactcagccggcatccgtgtccggtagccccggacagtcgattaccatctcctgtaccggcacctcctccgacgtgggagggtacaactacgtgtcgtggtaccagcagcacccaggaaaggcccctaagttgatgatctacgatgtgtcaaaccgcccgtctggagtctccaaccggttctccggctccaagtccggcaacaccgccagcctgaccattagcgggctgcaagccgaggatgaggccgactactactgctcgagctacacatcctcgagcaccctctacgtgttcggctcggggactaaggtcaccgtgctgaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 109 - protein, 17 aa, synthetic construct
  REGION 1..17: note = source = /note="Description of Artificial Sequence: Syntheticpeptide"
VISYKGSNKYYADSVKG

SEQ ID NO: 110 - protein, 6 aa, synthetic construct
  REGION 1..6: note = source = /note="Description of Artificial Sequence: Syntheticpeptide"
SYKGSN

SEQ ID NO: 111 - protein, 8 aa, synthetic construct
  REGION 1..8: note = source = /note="Description of Artificial Sequence: Syntheticpeptide"
ISYKGSNK

SEQ ID NO: 112 - protein, 123 aa, synthetic construct
  REGION 1..123: note = source = /note="Description of Artificial Sequence: Syntheticpolypeptide"
QVQLVESGGGVVQPGRSLRLSCAASGFTFSSYGMHWVRQAPGKGLEWVAVISYKGSNKYYADSVKGRFTISRDNSKNTLYLQMNSLRAEDTAVYYCGGSGYALHDDYYGLDVWGQGTLVTVSS

SEQ ID NO: 113 - DNA, 369 bp, synthetic construct
  misc_feature 1..369: note = source = /note="Description of Artificial Sequence: Syntheticpolynucleotide"
  source 1..369: mol_type = other DNA
caagtgcagcttgtcgaatcgggaggcggagtggtgcagcctggacgatcgctccggctctcatgtgccgcgagcggattcaccttctcgagctacggcatgcactgggtcagacaagccccaggaaagggcctggaatgggtggctgtcatctcgtacaagggctcaaacaagtactacgccgactccgtgaagggccggttcaccatctcccgcgataactccaagaataccctctatctgcaaatgaacagcctgagggccgaggatactgcagtgtactactgcgggggttcaggctacgcgctgcacgacgactactacggattggacgtctggggccaaggaactcttgtgaccgtgtcctct

SEQ ID NO: 114 - protein, 7 aa, synthetic construct
  REGION 1..7: note = source = /note="Description of Artificial Sequence: Syntheticpeptide"
EVSNRLR

SEQ ID NO: 115 - protein, 11 aa, synthetic construct
  REGION 1..11: note = source = /note="Description of Artificial Sequence: Syntheticpeptide"
SSYTSSSALYV

SEQ ID NO: 116 - skipped

SEQ ID NO: 117 - protein, 8 aa, synthetic construct
  REGION 1..8: note = source = /note="Description of Artificial Sequence: Syntheticpeptide"
YTSSSALY

SEQ ID NO: 118 - protein, 111 aa, synthetic construct
  REGION 1..111: note = source = /note="Description of Artificial Sequence: Syntheticpolypeptide"
QSALTQPASVSGSPGQSITISCTGTSSDVGGYNYVSWYQQHPGKAPKLMIYEVSNRLRGVSNRFSGSKSGNTASLTISGLQAEDEADYYCSSYTSSSALYVFGSGTKVTVL

SEQ ID NO: 119 - DNA, 333 bp, synthetic construct
  misc_feature 1..333: note = source = /note="Description of Artificial Sequence: Syntheticpolynucleotide"
  source 1..333: mol_type = other DNA
cagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccgccctctacgtgttcgggtccgggaccaaagtcactgtgctg

SEQ ID NO: 120 - protein, 254 aa, synthetic construct
  REGION 1..254: note = source = /note="Description of Artificial Sequence: Syntheticpolypeptide"
QVQLVESGGGVVQPGRSLRLSCAASGFTFSSYGMHWVRQAPGKGLEWVAVISYKGSNKYYADSVKGRFTISRDNSKNTLYLQMNSLRAEDTAVYYCGGSGYALHDDYYGLDVWGQGTLVTVSSGGGGSGGGGSGGGGSGGGGSQSALTQPASVSGSPGQSITISCTGTSSDVGGYNYVSWYQQHPGKAPKLMIYEVSNRLRGVSNRFSGSKSGNTASLTISGLQAEDEADYYCSSYTSSSALYVFGSGTKVTVL

SEQ ID NO: 121 - DNA, 762 bp, synthetic construct
  misc_feature 1..762: note = source = /note="Description of Artificial Sequence: Syntheticpolynucleotide"
  source 1..762: mol_type = other DNA
caagtgcagcttgtcgaatcgggaggcggagtggtgcagcctggacgatcgctccggctctcatgtgccgcgagcggattcaccttctcgagctacggcatgcactgggtcagacaagccccaggaaagggcctggaatgggtggctgtcatctcgtacaagggctcaaacaagtactacgccgactccgtgaagggccggttcaccatctcccgcgataactccaagaataccctctatctgcaaatgaacagcctgagggccgaggatactgcagtgtactactgcgggggttcaggctacgcgctgcacgacgactactacggattggacgtctggggccaaggaactcttgtgaccgtgtcctctggtggaggcggatcagggggtggcggatctgggggtggtggttccgggggaggaggatcgcagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccgccctctacgtgttcgggtccgggaccaaagtcactgtgctg

SEQ ID NO: 122 - protein, 477 aa, synthetic construct
  REGION 1..477: note = source = /note="Description of Artificial Sequence: Syntheticpolypeptide"
QVQLVESGGGVVQPGRSLRLSCAASGFTFSSYGMHWVRQAPGKGLEWVAVISYKGSNKYYADSVKGRFTISRDNSKNTLYLQMNSLRAEDTAVYYCGGSGYALHDDYYGLDVWGQGTLVTVSSGGGGSGGGGSGGGGSGGGGSQSALTQPASVSGSPGQSITISCTGTSSDVGGYNYVSWYQQHPGKAPKLMIYEVSNRLRGVSNRFSGSKSGNTASLTISGLQAEDEADYYCSSYTSSSALYVFGSGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - DNA, 1431 bp, synthetic construct
  misc_feature 1..1431: note = source = /note="Description of Artificial Sequence: Syntheticpolynucleotide"
  source 1..1431: mol_type = other DNA
caagtgcagcttgtcgaatcgggaggcggagtggtgcagcctggacgatcgctccggctctcatgtgccgcgagcggattcaccttctcgagctacggcatgcactgggtcagacaagccccaggaaagggcctggaatgggtggctgtcatctcgtacaagggctcaaacaagtactacgccgactccgtgaagggccggttcaccatctcccgcgataactccaagaataccctctatctgcaaatgaacagcctgagggccgaggatactgcagtgtactactgcgggggttcaggctacgcgctgcacgacgactactacggattggacgtctggggccaaggaactcttgtgaccgtgtcctctggtggaggcggatcagggggtggcggatctgggggtggtggttccgggggaggaggatcgcagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccgccctctacgtgttcgggtccgggaccaaagtcactgtgctgaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 124 - protein, 111 aa, synthetic construct
  REGION 1..111: note = source = /note="Description of Artificial Sequence: Syntheticpolypeptide"
QSALTQPASVSGSPGQSITISCTGTSSDVGGYNYVSWYQQHPGKAPKLMIYEVSNRLRGVSNRFSGSKSGNTASLTISGLQAEDEADYYCSSYTSSSTLYVFGSGTKVTVL

SEQ ID NO: 125 - DNA, 333 bp, synthetic construct
  misc_feature 1..333: note = source = /note="Description of Artificial Sequence: Syntheticpolynucleotide"
  source 1..333: mol_type = other DNA
cagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccaccctctacgtgttcgggtccgggaccaaagtcactgtgctg

SEQ ID NO: 126 - protein, 254 aa, synthetic construct
  REGION 1..254: note = source = /note="Description of Artificial Sequence: Syntheticpolypeptide"
QVQLVESGGGVVQPGRSLRLSCAASGFTFSSYGMHWVRQAPGKGLEWVAVISYKGSNKYYADSVKGRFTISRDNSKNTLYLQMNSLRAEDTAVYYCGGSGYALHDDYYGLDVWGQGTLVTVSSGGGGSGGGGSGGGGSGGGGSQSALTQPASVSGSPGQSITISCTGTSSDVGGYNYVSWYQQHPGKAPKLMIYEVSNRLRGVSNRFSGSKSGNTASLTISGLQAEDEADYYCSSYTSSSTLYVFGSGTKVTVL

SEQ ID NO: 127 - DNA, 762 bp, synthetic construct
  misc_feature 1..762: note = source = /note="Description of Artificial Sequence: Syntheticpolynucleotide"
  source 1..762: mol_type = other DNA
caagtgcagcttgtcgaatcgggaggcggagtggtgcagcctggacgatcgctccggctctcatgtgccgcgagcggattcaccttctcgagctacggcatgcactgggtcagacaagccccaggaaagggcctggaatgggtggctgtcatctcgtacaagggctcaaacaagtactacgccgactccgtgaagggccggttcaccatctcccgcgataactccaagaataccctctatctgcaaatgaacagcctgagggccgaggatactgcagtgtactactgcgggggttcaggctacgcgctgcacgacgactactacggattggacgtctggggccaaggaactcttgtgaccgtgtcctctggtggaggcggatcagggggtggcggatctgggggtggtggttccgggggaggaggatcgcagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccaccctctacgtgttcgggtccgggaccaaagtcactgtgctg

SEQ ID NO: 128 - protein, 477 aa, synthetic construct
  REGION 1..477: note = source = /note="Description of Artificial Sequence: Syntheticpolypeptide"
QVQLVESGGGVVQPGRSLRLSCAASGFTFSSYGMHWVRQAPGKGLEWVAVISYKGSNKYYADSVKGRFTISRDNSKNTLYLQMNSLRAEDTAVYYCGGSGYALHDDYYGLDVWGQGTLVTVSSGGGGSGGGGSGGGGSGGGGSQSALTQPASVSGSPGQSITISCTGTSSDVGGYNYVSWYQQHPGKAPKLMIYEVSNRLRGVSNRFSGSKSGNTASLTISGLQAEDEADYYCSSYTSSSTLYVFGSGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - DNA, 1431 bp, synthetic construct
  misc_feature 1..1431: note = source = /note="Description of Artificial Sequence: Syntheticpolynucleotide"
  source 1..1431: mol_type = other DNA
caagtgcagcttgtcgaatcgggaggcggagtggtgcagcctggacgatcgctccggctctcatgtgccgcgagcggattcaccttctcgagctacggcatgcactgggtcagacaagccccaggaaagggcctggaatgggtggctgtcatctcgtacaagggctcaaacaagtactacgccgactccgtgaagggccggttcaccatctcccgcgataactccaagaataccctctatctgcaaatgaacagcctgagggccgaggatactgcagtgtactactgcgggggttcaggctacgcgctgcacgacgactactacggattggacgtctggggccaaggaactcttgtgaccgtgtcctctggtggaggcggatcagggggtggcggatctgggggtggtggttccgggggaggaggatcgcagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccaccctctacgtgttcgggtccgggaccaaagtcactgtgctgaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 130 - protein, 17 aa, synthetic construct
  VARIANT 5: note = /replace="Lys"
  SITE 1..17: note = /note="Variant residues given in the sequence have nopreference with respect to those in the annotationsfor variant positions"
  REGION 1..17: note = source = /note="Description of Artificial Sequence: Syntheticpeptide"
VISYDGSNKYYADSVKG

SEQ ID NO: 131 - protein, 7 aa, synthetic construct
  VARIANT 1: note = /replace="Glu"
  VARIANT 6: note = /replace="Leu"
  VARIANT 7: note = /replace="Arg"
  SITE 1..7: note = /note="Variant residues given in the sequence have nopreference with respect to those in the annotationsfor variant positions"
  REGION 1..7: note = source = /note="Description of Artificial Sequence: Syntheticpeptide"
DVSNRPS

SEQ ID NO: 132 - protein, 11 aa, synthetic construct
  VARIANT 8: note = /replace="Ala"
  SITE 1..11: note = /note="Variant residues given in the sequence have nopreference with respect to those in the annotationsfor variant positions"
  REGION 1..11: note = source = /note="Description of Artificial Sequence: Syntheticpeptide"
SSYTSSSTLYV

SEQ ID NO: 133 - protein, 6 aa, synthetic construct
  VARIANT 3: note = /replace="Lys"
  SITE 1..6: note = /note="Variant residues given in the sequence have nopreference with respect to those in the annotationsfor variant positions"
  REGION 1..6: note = source = /note="Description of Artificial Sequence: Syntheticpeptide"
SYDGSN

SEQ ID NO: 134 - skipped

SEQ ID NO: 135 - protein, 8 aa, synthetic construct
  VARIANT 6: note = /replace="Ala"
  SITE 1..8: note = /note="Variant residues given in the sequence have nopreference with respect to those in the annotationsfor variant positions"
  REGION 1..8: note = source = /note="Description of Artificial Sequence: Syntheticpeptide"
YTSSSTLY

SEQ ID NO: 136 - protein, 8 aa, synthetic construct
  VARIANT 4: note = /replace="Lys"
  SITE 1..8: note = /note="Variant residues given in the sequence have nopreference with respect to those in the annotationsfor variant positions"
  REGION 1..8: note = source = /note="Description of Artificial Sequence: Syntheticpeptide"
ISYDGSNK

SEQ ID NO: 137 - protein, 5 aa, synthetic construct
  REGION 1..5: note = source = /note="Description of Artificial Sequence: Syntheticpeptide"
GFWMS

SEQ ID NO: 138 - protein, 17 aa, synthetic construct
  REGION 1..17: note = source = /note="Description of Artificial Sequence: Syntheticpeptide"
NIKQDGSEKYYVDSVRG

SEQ ID NO: 139 - protein, 9 aa, synthetic construct
  REGION 1..9: note = source = /note="Description of Artificial Sequence: Syntheticpeptide"
ALDYYGMDV

SEQ ID NO: 140 - protein, 7 aa, synthetic construct
  REGION 1..7: note = source = /note="Description of Artificial Sequence: Syntheticpeptide"
GFTFSGF

SEQ ID NO: 141 - protein, 6 aa, synthetic construct
  REGION 1..6: note = source = /note="Description of Artificial Sequence: Syntheticpeptide"
KQDGSE

SEQ ID NO: 142 - protein, 8 aa, synthetic construct
  REGION 1..8: note = source = /note="Description of Artificial Sequence: Syntheticpeptide"
GFTFSGFW

SEQ ID NO: 143 - protein, 8 aa, synthetic construct
  REGION 1..8: note = source = /note="Description of Artificial Sequence: Syntheticpeptide"
IKQDGSEK

SEQ ID NO: 144 - protein, 11 aa, synthetic construct
  REGION 1..11: note = source = /note="Description of Artificial Sequence: Syntheticpeptide"
ARALDYYGMDV

SEQ ID NO: 145 - protein, 118 aa, synthetic construct
  REGION 1..118: note = source = /note="Description of Artificial Sequence: Syntheticpolypeptide"
EVQLVESGGGLVQPGGSLRLSCAASGFTFSGFWMSWVRQAPGKGLEWVANIKQDGSEKYYVDSVRGRFTISRDNAKNSLYLQMNSLRAEDTAVYYCARALDYYGMDVWGQGTTVTVSS

SEQ ID NO: 146 - DNA, 354 bp, synthetic construct
  misc_feature 1..354: note = source = /note="Description of Artificial Sequence: Syntheticpolynucleotide"
  source 1..354: mol_type = other DNA
gaagtgcaactggtggagagcggtggagggcttgtccagcccggaggatcgctgcggctgtcctgtgctgcgtccgggttcaccttctccggcttctggatgtcctgggtcagacaggcaccgggaaagggcctcgaatgggtggccaacatcaagcaggatggctccgagaagtactacgtcgactccgtgagaggccgcttcaccatctcccgggacaacgccaagaactcgctgtacctccaaatgaatagcctcagggcggaagatactgctgtgtattactgcgcacgcgcccttgactactacggcatggacgtctggggccaagggaccactgtgaccgtgtctagc

SEQ ID NO: 147 - protein, 17 aa, synthetic construct
  REGION 1..17: note = source = /note="Description of Artificial Sequence: Syntheticpeptide"
RSSQSLLDSDDGNTYLD

SEQ ID NO: 148 - protein, 7 aa, synthetic construct
  REGION 1..7: note = source = /note="Description of Artificial Sequence: Syntheticpeptide"
TLSYRAS

SEQ ID NO: 149 - protein, 10 aa, synthetic construct
  REGION 1..10: note = source = /note="Description of Artificial Sequence: Syntheticpeptide"
TQRLEFPSIT

SEQ ID NO: 150 - protein, 13 aa, synthetic construct
  REGION 1..13: note = source = /note="Description of Artificial Sequence: Syntheticpeptide"
SQSLLDSDDGNTY

SEQ ID NO: 151 - skipped

SEQ ID NO: 152 - protein, 7 aa, synthetic construct
  REGION 1..7: note = source = /note="Description of Artificial Sequence: Syntheticpeptide"
RLEFPSI

SEQ ID NO: 153 - protein, 12 aa, synthetic construct
  REGION 1..12: note = source = /note="Description of Artificial Sequence: Syntheticpeptide"
QSLLDSDDGNTY

SEQ ID NO: 154 - protein, 114 aa, synthetic construct
  REGION 1..114: note = source = /note="Description of Artificial Sequence: Syntheticpolypeptide"
DIVMTQTPLSLPVTPGEPASISCRSSQSLLDSDDGNTYLDWYLQKPGQSPRLLIYTLSYRASGVPDRFSGSGSGTDFTLKISRVEAEDVGLYYCTQRLEFPSITFGQGTRLEIK

SEQ ID NO: 155 - DNA, 342 bp, synthetic construct
  misc_feature 1..342: note = source = /note="Description of Artificial Sequence: Syntheticpolynucleotide"
  source 1..342: mol_type = other DNA
gatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcgcctgctgatctataccctgtcataccgggcctcaggagtgcctgaccgcttctcgggatcagggagcgggaccgatttcaccctgaaaatttcccgagtggaagccgaggacgtcggactgtactactgcacccagcgcctcgaattcccgtcgattacgtttggacagggtacccggcttgagatcaag

SEQ ID NO: 156 - protein, 252 aa, synthetic construct
  REGION 1..252: note = source = /note="Description of Artificial Sequence: Syntheticpolypeptide"
EVQLVESGGGLVQPGGSLRLSCAASGFTFSGFWMSWVRQAPGKGLEWVANIKQDGSEKYYVDSVRGRFTISRDNAKNSLYLQMNSLRAEDTAVYYCARALDYYGMDVWGQGTTVTVSSGGGGSGGGGSGGGGSGGGGSDIVMTQTPLSLPVTPGEPASISCRSSQSLLDSDDGNTYLDWYLQKPGQSPRLLIYTLSYRASGVPDRFSGSGSGTDFTLKISRVEAEDVGLYYCTQRLEFPSITFGQGTRLEIK

SEQ ID NO: 157 - DNA, 756 bp, synthetic construct
  misc_feature 1..756: note = source = /note="Description of Artificial Sequence: Syntheticpolynucleotide"
  source 1..756: mol_type = other DNA
gaagtgcaactggtggagagcggtggagggcttgtccagcccggaggatcgctgcggctgtcctgtgctgcgtccgggttcaccttctccggcttctggatgtcctgggtcagacaggcaccgggaaagggcctcgaatgggtggccaacatcaagcaggatggctccgagaagtactacgtcgactccgtgagaggccgcttcaccatctcccgggacaacgccaagaactcgctgtacctccaaatgaatagcctcagggcggaagatactgctgtgtattactgcgcacgcgcccttgactactacggcatggacgtctggggccaagggaccactgtgaccgtgtctagcggaggcggaggttcagggggcggtggatcaggcggaggaggatcggggggtggtggatcggatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcgcctgctgatctataccctgtcataccgggcctcaggagtgcctgaccgcttctcgggatcagggagcgggaccgatttcaccctgaaaatttcccgagtggaagccgaggacgtcggactgtactactgcacccagcgcctcgaattcccgtcgattacgtttggacagggtacccggcttgagatcaag

SEQ ID NO: 158 - protein, 475 aa, synthetic construct
  REGION 1..475: note = source = /note="Description of Artificial Sequence: Syntheticpolypeptide"
EVQLVESGGGLVQPGGSLRLSCAASGFTFSGFWMSWVRQAPGKGLEWVANIKQDGSEKYYVDSVRGRFTISRDNAKNSLYLQMNSLRAEDTAVYYCARALDYYGMDVWGQGTTVTVSSGGGGSGGGGSGGGGSGGGGSDIVMTQTPLSLPVTPGEPASISCRSSQSLLDSDDGNTYLDWYLQKPGQSPRLLIYTLSYRASGVPDRFSGSGSGTDFTLKISRVEAEDVGLYYCTQRLEFPSITFGQGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 159 - DNA, 1425 bp, synthetic construct
  misc_feature 1..1425: note = source = /note="Description of Artificial Sequence: Syntheticpolynucleotide"
  source 1..1425: mol_type = other DNA
gaagtgcaactggtggagagcggtggagggcttgtccagcccggaggatcgctgcggctgtcctgtgctgcgtccgggttcaccttctccggcttctggatgtcctgggtcagacaggcaccgggaaagggcctcgaatgggtggccaacatcaagcaggatggctccgagaagtactacgtcgactccgtgagaggccgcttcaccatctcccgggacaacgccaagaactcgctgtacctccaaatgaatagcctcagggcggaagatactgctgtgtattactgcgcacgcgcccttgactactacggcatggacgtctggggccaagggaccactgtgaccgtgtctagcggaggcggaggttcagggggcggtggatcaggcggaggaggatcggggggtggtggatcggatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcgcctgctgatctataccctgtcataccgggcctcaggagtgcctgaccgcttctcgggatcagggagcgggaccgatttcaccctgaaaatttcccgagtggaagccgaggacgtcggactgtactactgcacccagcgcctcgaattcccgtcgattacgtttggacagggtacccggcttgagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 160 - protein, 5 aa, synthetic construct
  REGION 1..5: note = source = /note="Description of Artificial Sequence: Syntheticpeptide"
SFRMN

SEQ ID NO: 161 - protein, 17 aa, synthetic construct
  REGION 1..17: note = source = /note="Description of Artificial Sequence: Syntheticpeptide"
SISSSSSYIYYADSVKG

SEQ ID NO: 162 - protein, 9 aa, synthetic construct
  REGION 1..9: note = source = /note="Description of Artificial Sequence: Syntheticpeptide"
WLSYYGMDV

SEQ ID NO: 163 - protein, 7 aa, synthetic construct
  REGION 1..7: note = source = /note="Description of Artificial Sequence: Syntheticpeptide"
GFTFSSF

SEQ ID NO: 164 - protein, 6 aa, synthetic construct
  REGION 1..6: note = source = /note="Description of Artificial Sequence: Syntheticpeptide"
SSSSSY

SEQ ID NO: 165 - protein, 8 aa, synthetic construct
  REGION 1..8: note = source = /note="Description of Artificial Sequence: Syntheticpeptide"
GFTFSSFR

SEQ ID NO: 166 - protein, 8 aa, synthetic construct
  REGION 1..8: note = source = /note="Description of Artificial Sequence: Syntheticpeptide"
ISSSSSYI

SEQ ID NO: 167 - protein, 11 aa, synthetic construct
  REGION 1..11: note = source = /note="Description of Artificial Sequence: Syntheticpeptide"
ARWLSYYGMDV

SEQ ID NO: 168 - protein, 118 aa, synthetic construct
  REGION 1..118: note = source = /note="Description of Artificial Sequence: Syntheticpolypeptide"
EVQLVESGGGLVKPGGSLRLSCAASGFTFSSFRMNWVRQAPGKGLEWVSSISSSSSYIYYADSVKGRFTISRDNAKNSLYLQMNSLRAEDTAVYYCARWLSYYGMDVWGQGTTVTVSS

SEQ ID NO: 169 - DNA, 354 bp, synthetic construct
  misc_feature 1..354: note = source = /note="Description of Artificial Sequence: Syntheticpolynucleotide"
  source 1..354: mol_type = other DNA
gaagtgcaactggtggagagcggtggagggcttgtcaagcccggaggatcgctgcggctgtcctgtgctgcgtccgggttcaccttctcctcgttccgcatgaactgggtcagacaggcaccgggaaagggcctcgaatgggtgtcctcaatctcatcgtcctcgtcctacatctactacgccgactccgtgaaaggccgcttcaccatctcccgggacaacgccaagaactcgctgtacctccaaatgaatagcctcagggcggaagatactgctgtgtattactgcgcacgctggctttcctactacggcatggacgtctggggccaagggaccactgtgaccgtgtctagc

SEQ ID NO: 170 - protein, 7 aa, synthetic construct
  REGION 1..7: note = source = /note="Description of Artificial Sequence: Syntheticpeptide"
TLSFRAS

SEQ ID NO: 171 - protein, 9 aa, synthetic construct
  REGION 1..9: note = source = /note="Description of Artificial Sequence: Syntheticpeptide"
MQRIGFPIT

SEQ ID NO: 172 - protein, 6 aa, synthetic construct
  REGION 1..6: note = source = /note="Description of Artificial Sequence: Syntheticpeptide"
RIGFPI

SEQ ID NO: 173 - protein, 113 aa, synthetic construct
  REGION 1..113: note = source = /note="Description of Artificial Sequence: Syntheticpolypeptide"
DIVMTQTPLSLPVTPGEPASISCRSSQSLLDSDDGNTYLDWYLQKPGQSPQLLIYTLSFRASGVPDRFSGSGSGTDFTLKIRRVEAEDVGVYYCMQRIGFPITFGQGTRLEIK

SEQ ID NO: 174 - DNA, 339 bp, synthetic construct
  misc_feature 1..339: note = source = /note="Description of Artificial Sequence: Syntheticpolynucleotide"
  source 1..339: mol_type = other DNA
gatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcagctgctgatctataccctgtcattccgggcctcaggagtgcctgaccgcttctcgggatcagggagcgggaccgatttcaccctgaaaattaggcgagtggaagccgaggacgtcggagtgtactactgcatgcagcgcatcggcttcccgattacgtttggacagggtacccggcttgagatcaag

SEQ ID NO: 175 - protein, 251 aa, synthetic construct
  REGION 1..251: note = source = /note="Description of Artificial Sequence: Syntheticpolypeptide"
EVQLVESGGGLVKPGGSLRLSCAASGFTFSSFRMNWVRQAPGKGLEWVSSISSSSSYIYYADSVKGRFTISRDNAKNSLYLQMNSLRAEDTAVYYCARWLSYYGMDVWGQGTTVTVSSGGGGSGGGGSGGGGSGGGGSDIVMTQTPLSLPVTPGEPASISCRSSQSLLDSDDGNTYLDWYLQKPGQSPQLLIYTLSFRASGVPDRFSGSGSGTDFTLKIRRVEAEDVGVYYCMQRIGFPITFGQGTRLEIK

SEQ ID NO: 176 - DNA, 753 bp, synthetic construct
  misc_feature 1..753: note = source = /note="Description of Artificial Sequence: Syntheticpolynucleotide"
  source 1..753: mol_type = other DNA
gaagtgcaactggtggagagcggtggagggcttgtcaagcccggaggatcgctgcggctgtcctgtgctgcgtccgggttcaccttctcctcgttccgcatgaactgggtcagacaggcaccgggaaagggcctcgaatgggtgtcctcaatctcatcgtcctcgtcctacatctactacgccgactccgtgaaaggccgcttcaccatctcccgggacaacgccaagaactcgctgtacctccaaatgaatagcctcagggcggaagatactgctgtgtattactgcgcacgctggctttcctactacggcatggacgtctggggccaagggaccactgtgaccgtgtctagcggaggcggaggttcagggggcggtggatcaggcggaggaggatcggggggtggtggatcggatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcagctgctgatctataccctgtcattccgggcctcaggagtgcctgaccgcttctcgggatcagggagcgggaccgatttcaccctgaaaattaggcgagtggaagccgaggacgtcggagtgtactactgcatgcagcgcatcggcttcccgattacgtttggacagggtacccggcttgagatcaag

SEQ ID NO: 177 - protein, 474 aa, synthetic construct
  REGION 1..474: note = source = /note="Description of Artificial Sequence: Syntheticpolypeptide"
EVQLVESGGGLVKPGGSLRLSCAASGFTFSSFRMNWVRQAPGKGLEWVSSISSSSSYIYYADSVKGRFTISRDNAKNSLYLQMNSLRAEDTAVYYCARWLSYYGMDVWGQGTTVTVSSGGGGSGGGGSGGGGSGGGGSDIVMTQTPLSLPVTPGEPASISCRSSQSLLDSDDGNTYLDWYLQKPGQSPQLLIYTLSFRASGVPDRFSGSGSGTDFTLKIRRVEAEDVGVYYCMQRIGFPITFGQGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 178 - DNA, 1422 bp, synthetic construct
  misc_feature 1..1422: note = source = /note="Description of Artificial Sequence: Syntheticpolynucleotide"
  source 1..1422: mol_type = other DNA
gaagtgcaactggtggagagcggtggagggcttgtcaagcccggaggatcgctgcggctgtcctgtgctgcgtccgggttcaccttctcctcgttccgcatgaactgggtcagacaggcaccgggaaagggcctcgaatgggtgtcctcaatctcatcgtcctcgtcctacatctactacgccgactccgtgaaaggccgcttcaccatctcccgggacaacgccaagaactcgctgtacctccaaatgaatagcctcagggcggaagatactgctgtgtattactgcgcacgctggctttcctactacggcatggacgtctggggccaagggaccactgtgaccgtgtctagcggaggcggaggttcagggggcggtggatcaggcggaggaggatcggggggtggtggatcggatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcagctgctgatctataccctgtcattccgggcctcaggagtgcctgaccgcttctcgggatcagggagcgggaccgatttcaccctgaaaattaggcgagtggaagccgaggacgtcggagtgtactactgcatgcagcgcatcggcttcccgattacgtttggacagggtacccggcttgagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 179 - protein, 5 aa, synthetic construct
  VARIANT 1: note = /replace="Ser"
  VARIANT 3: note = /replace="Arg"
  VARIANT 5: note = /replace="Asn"
  SITE 1..5: note = /note="Variant residues given in the sequence have nopreference with respect to those in the annotationsfor variant positions"
  REGION 1..5: note = source = /note="Description of Artificial Sequence: Syntheticpeptide"
GFWMS

SEQ ID NO: 180 - protein, 17 aa, synthetic construct
  VARIANT 1: note = /replace="Ser"
  VARIANT 3: note = /replace="Ser"
  VARIANT 4: note = /replace="Ser"
  VARIANT 5: note = /replace="Ser"
  VARIANT 6: note = /replace="Ser"
  VARIANT 8: note = /replace="Tyr"
  VARIANT 9: note = /replace="Ile"
  VARIANT 12: note = /replace="Ala"
  VARIANT 16: note = /replace="Lys"
  SITE 1..17: note = /note="Variant residues given in the sequence have nopreference with respect to those in the annotationsfor variant positions"
  REGION 1..17: note = source = /note="Description of Artificial Sequence: Syntheticpeptide"
NIKQDGSEKYYVDSVRG

SEQ ID NO: 181 - protein, 9 aa, synthetic construct
  VARIANT 1: note = /replace="Trp"
  VARIANT 3: note = /replace="Ser"
  SITE 1..9: note = /note="Variant residues given in the sequence have nopreference with respect to those in the annotationsfor variant positions"
  REGION 1..9: note = source = /note="Description of Artificial Sequence: Syntheticpeptide"
ALDYYGMDV

SEQ ID NO: 182 - protein, 7 aa, synthetic construct
  VARIANT 4: note = /replace="Phe"
  SITE 1..7: note = /note="Variant residues given in the sequence have nopreference with respect to those in the annotationsfor variant positions"
  REGION 1..7: note = source = /note="Description of Artificial Sequence: Syntheticpeptide"
TLSYRAS

SEQ ID NO: 183 - protein, 10 aa, synthetic construct
  VARIANT 1: note = /replace="Met"
  VARIANT 4: note = /replace="Ile"
  VARIANT 5: note = /replace="Gly"
  VARIANT 8: note = /replace=" "
  SITE 1..10: note = /note="Variant residues given in the sequence have nopreference with respect to those in the annotationsfor variant positions"
  REGION 1..10: note = source = /note="Description of Artificial Sequence: Syntheticpeptide"
TQRLEFPSIT

SEQ ID NO: 184 - protein, 7 aa, synthetic construct
  VARIANT 6: note = /replace="Ser"
  SITE 1..7: note = /note="Variant residues given in the sequence have nopreference with respect to those in the annotationsfor variant positions"
  REGION 1..7: note = source = /note="Description of Artificial Sequence: Syntheticpeptide"
GFTFSGF

SEQ ID NO: 185 - protein, 6 aa, synthetic construct
  VARIANT 1: note = /replace="Ser"
  VARIANT 2: note = /replace="Ser"
  VARIANT 3: note = /replace="Ser"
  VARIANT 4: note = /replace="Ser"
  VARIANT 6: note = /replace="Tyr"
  SITE 1..6: note = /note="Variant residues given in the sequence have nopreference with respect to those in the annotationsfor variant positions"
  REGION 1..6: note = source = /note="Description of Artificial Sequence: Syntheticpeptide"
KQDGSE

SEQ ID NO: 186 - protein, 7 aa, synthetic construct
  VARIANT 2: note = /replace="Ile"
  VARIANT 3: note = /replace="Gly"
  VARIANT 6: note = /replace=" "
  SITE 1..7: note = /note="Variant residues given in the sequence have nopreference with respect to those in the annotationsfor variant positions"
  REGION 1..7: note = source = /note="Description of Artificial Sequence: Syntheticpeptide"
RLEFPSI

SEQ ID NO: 187 - protein, 8 aa, synthetic construct
  VARIANT 6: note = /replace="Ser"
  VARIANT 8: note = /replace="Arg"
  SITE 1..8: note = /note="Variant residues given in the sequence have nopreference with respect to those in the annotationsfor variant positions"
  REGION 1..8: note = source = /note="Description of Artificial Sequence: Syntheticpeptide"
GFTFSGFW

SEQ ID NO: 188 - protein, 8 aa, synthetic construct
  VARIANT 2: note = /replace="Ser"
  VARIANT 3: note = /replace="Ser"
  VARIANT 4: note = /replace="Ser"
  VARIANT 5: note = /replace="Ser"
  VARIANT 7: note = /replace="Tyr"
  VARIANT 8: note = /replace="Ile"
  SITE 1..8: note = /note="Variant residues given in the sequence have nopreference with respect to those in the annotationsfor variant positions"
  REGION 1..8: note = source = /note="Description of Artificial Sequence: Syntheticpeptide"
IKQDGSEK

SEQ ID NO: 189 - protein, 11 aa, synthetic construct
  VARIANT 3: note = /replace="Trp"
  VARIANT 5: note = /replace="Ser"
  SITE 1..11: note = /note="Variant residues given in the sequence have nopreference with respect to those in the annotationsfor variant positions"
  REGION 1..11: note = source = /note="Description of Artificial Sequence: Syntheticpeptide"
ARALDYYGMDV

SEQ ID NO: 190 - DNA, 521 bp, unidentified
  misc_feature 1..521: note = source = /note="Description of Unknown:PGK Promoter sequence"
  source 1..521: mol_type = other DNA
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccgcggcgacgcaaagggccttggtgcgggtctcgtcggcgcagggacgcgtttgggtcccgacggaaccttttccgcgttggggttggggcaccataagct

SEQ ID NO: 191 - DNA, 221 bp, synthetic construct
  misc_feature 1..221: note = source = /note="Description of Artificial Sequence: Syntheticpolynucleotide"
  source 1..221: mol_type = other DNA
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacg

SEQ ID NO: 192 - DNA, 324 bp, synthetic construct
  misc_feature 1..324: note = source = /note="Description of Artificial Sequence: Syntheticpolynucleotide"
  source 1..324: mol_type = other DNA
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccg

SEQ ID NO: 193 - DNA, 422 bp, synthetic construct
  misc_feature 1..422: note = source = /note="Description of Artificial Sequence: Syntheticpolynucleotide"
  source 1..422: mol_type = other DNA
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccg

SEQ ID NO: 194-197 - skipped (4 entries)

SEQ ID NO: 198 - DNA, 118 bp, synthetic construct
  misc_feature 1..118: note = source = /note="Description of Artificial Sequence: Syntheticpolynucleotide"
  source 1..118: mol_type = other DNA
acccctctctccagccactaagccagttgctccctcggctgacggctgcacgcgaggcctccgaacgtcttacgccttgtggcgcgcccgtccttgtcccgggtgtgatggcggggtg

SEQ ID NO: 199 - DNA, 63 bp, synthetic construct
  misc_feature 1..63: note = source = /note="Description of Artificial Sequence: Syntheticoligonucleotide"
  source 1..63: mol_type = other DNA
atggccctccctgtcaccgccctgctgcttccgctggctcttctgctccacgccgctcggccc

SEQ ID NO: 200-201 - skipped (2 entries)

SEQ ID NO: 202 - protein, 69 aa, synthetic construct
  REGION 1..69: note = source = /note="Description of Artificial Sequence: Syntheticpolypeptide"
TTTPAPRPPTPAPTIASQPLSLRPEACRPAAGGAVHTRGLDFACDIYIWAPLAGTCGVLLLSLVITLYC

SEQ ID NO: 203-224 - skipped (22 entries)

SEQ ID NO: 225 - protein, 465 aa, synthetic construct
  REGION 1..465: note = source = /note="Description of Artificial Sequence: Syntheticpolypeptide"
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 226-249 - skipped (24 entries)

SEQ ID NO: 250 - protein, 120 aa, synthetic construct
  REGION 1..120: note = source = /note="Description of Artificial Sequence: Syntheticpolypeptide"
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 251 - protein, 107 aa, synthetic construct
  REGION 1..107: note = source = /note="Description of Artificial Sequence: Syntheticpolypeptide"
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 252 - DNA, 63 bp, synthetic construct
  misc_feature 1..63: note = source = /note="Description of Artificial Sequence: Syntheticoligonucleotide"
  source 1..63: mol_type = other DNA
atggccctccctgtcaccgctctgttgctgccgcttgctctgctgctccacgcagcgcgaccg

SEQ ID NO: 253 - DNA, 747 bp, synthetic construct
  misc_feature 1..747: note = source = /note="Description of Artificial Sequence: Syntheticpolynucleotide"
  source 1..747: mol_type = other DNA
caggtacaattgcaggagtctggaggcggtgtggtgcaacccggtcgcagcttgcgcctgagttgtgctgcgtctggatttacattttcatcttacggaatgcattgggtacgccaggcaccggggaaaggccttgaatgggtggctgtaatttcatacgatggttccaacaaatactatgctgactcagtcaagggtcgatttacaattagtcgggacaactccaagaacaccctttatcttcaaatgaattcccttagagcagaggatacggcggtctattactgtggtggcagtggttatgcacttcatgatgattactatggcttggatgtctgggggcaagggacgcttgtaactgtatcctctggtggtggtggtagtggtgggggaggctccggcggtggcggctctcaatctgctctgactcaaccagcaagcgtatcagggtcaccgggacagagtattaccataagttgcacggggacctctagcgatgtaggggggtataattatgtatcttggtatcaacaacaccccgggaaagcccctaaattgatgatctacgacgtgagcaatcgacctagtggcgtatcaaatcgcttctctggtagcaagagtgggaatacggcgtcccttactattagcggattgcaagcagaagatgaggccgattactactgcagctcctatactagctcttctacattgtacgtctttgggagcggaacaaaagtaacagtactc

SEQ ID NO: 254 - DNA, 207 bp, synthetic construct
  misc_feature 1..207: note = source = /note="Description of Artificial Sequence: Syntheticpolynucleotide"
  source 1..207: mol_type = other DNA
acaacaacacctgccccgagaccgcctacaccagccccgactattgccagccagcctctgagcctcaggcctgaggcctgtaggcccgcagcgggcggcgcagttcatacacggggcttggatttcgcttgtgatatttatatttgggctcctttggcggggacatgtggcgtgctgcttctgtcacttgttattacactgtactgt

SEQ ID NO: 255 - DNA, 126 bp, synthetic construct
  misc_feature 1..126: note = source = /note="Description of Artificial Sequence: Syntheticpolynucleotide"
  source 1..126: mol_type = other DNA
aaacgcgggcgaaaaaaattgctgtatatttttaagcagccatttatgaggcccgttcagacgacgcaggaggaggacggttgctcttgcaggttcccagaagaggaagaagggggctgtgaattg

SEQ ID NO: 256 - DNA, 336 bp, synthetic construct
  misc_feature 1..336: note = source = /note="Description of Artificial Sequence: Syntheticpolynucleotide"
  source 1..336: mol_type = other DNA
cgggttaaattttcaagatccgcagacgctccagcataccaacagggacaaaaccaactctataacgagctgaatcttggaagaagggaggaatatgatgtgctggataaacggcgcggtagagatccggagatgggcggaaaaccaaggcgaaaaaaccctcaggagggactctacaacgaactgcagaaagacaaaatggcggaggcttattccgaaataggcatgaagggcgagcggaggcgagggaaagggcacgacggactgtatcaaggcctctcaaccgcgactaaggatacgtacgacgccctgcacatgcaggccctgcctccgaga

SEQ ID NO: 257 - protein, 493 aa, synthetic construct
  REGION 1..493: note = source = /note="Description of Artificial Sequence: Syntheticpolypeptide"
MALPVTALLLPLALLLHAARPQVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 258 - DNA, 1479 bp, synthetic construct
  misc_feature 1..1479: note = source = /note="Description of Artificial Sequence: Syntheticpolynucleotide"
  source 1..1479: mol_type = other DNA
atggccctccctgtcaccgctctgttgctgccgcttgctctgctgctccacgcagcgcgaccgcaggtacaattgcaggagtctggaggcggtgtggtgcaacccggtcgcagcttgcgcctgagttgtgctgcgtctggatttacattttcatcttacggaatgcattgggtacgccaggcaccggggaaaggccttgaatgggtggctgtaatttcatacgatggttccaacaaatactatgctgactcagtcaagggtcgatttacaattagtcgggacaactccaagaacaccctttatcttcaaatgaattcccttagagcagaggatacggcggtctattactgtggtggcagtggttatgcacttcatgatgattactatggcttggatgtctgggggcaagggacgcttgtaactgtatcctctggtggtggtggtagtggtgggggaggctccggcggtggcggctctcaatctgctctgactcaaccagcaagcgtatcagggtcaccgggacagagtattaccataagttgcacggggacctctagcgatgtaggggggtataattatgtatcttggtatcaacaacaccccgggaaagcccctaaattgatgatctacgacgtgagcaatcgacctagtggcgtatcaaatcgcttctctggtagcaagagtgggaatacggcgtcccttactattagcggattgcaagcagaagatgaggccgattactactgcagctcctatactagctcttctacattgtacgtctttgggagcggaacaaaagtaacagtactcacaacaacacctgccccgagaccgcctacaccagccccgactattgccagccagcctctgagcctcaggcctgaggcctgtaggcccgcagcgggcggcgcagttcatacacggggcttggatttcgcttgtgatatttatatttgggctcctttggcggggacatgtggcgtgctgcttctgtcacttgttattacactgtactgtaaacgcgggcgaaaaaaattgctgtatatttttaagcagccatttatgaggcccgttcagacgacgcaggaggaggacggttgctcttgcaggttcccagaagaggaagaagggggctgtgaattgcgggttaaattttcaagatccgcagacgctccagcataccaacagggacaaaaccaactctataacgagctgaatcttggaagaagggaggaatatgatgtgctggataaacggcgcggtagagatccggagatgggcggaaaaccaaggcgaaaaaaccctcaggagggactctacaacgaactgcagaaagacaaaatggcggaggcttattccgaaataggcatgaagggcgagcggaggcgagggaaagggcacgacggactgtatcaaggcctctcaaccgcgactaaggatacgtacgacgccctgcacatgcaggccctgcctccgaga

SEQ ID NO: 259 - DNA, 1416 bp, synthetic construct
  misc_feature 1..1416: note = source = /note="Description of Artificial Sequence: Syntheticpolynucleotide"
  source 1..1416: mol_type = other DNA
caggtacaattgcaggagtctggaggcggtgtggtgcaacccggtcgcagcttgcgcctgagttgtgctgcgtctggatttacattttcatcttacggaatgcattgggtacgccaggcaccggggaaaggccttgaatgggtggctgtaatttcatacgatggttccaacaaatactatgctgactcagtcaagggtcgatttacaattagtcgggacaactccaagaacaccctttatcttcaaatgaattcccttagagcagaggatacggcggtctattactgtggtggcagtggttatgcacttcatgatgattactatggcttggatgtctgggggcaagggacgcttgtaactgtatcctctggtggtggtggtagtggtgggggaggctccggcggtggcggctctcaatctgctctgactcaaccagcaagcgtatcagggtcaccgggacagagtattaccataagttgcacggggacctctagcgatgtaggggggtataattatgtatcttggtatcaacaacaccccgggaaagcccctaaattgatgatctacgacgtgagcaatcgacctagtggcgtatcaaatcgcttctctggtagcaagagtgggaatacggcgtcccttactattagcggattgcaagcagaagatgaggccgattactactgcagctcctatactagctcttctacattgtacgtctttgggagcggaacaaaagtaacagtactcacaacaacacctgccccgagaccgcctacaccagccccgactattgccagccagcctctgagcctcaggcctgaggcctgtaggcccgcagcgggcggcgcagttcatacacggggcttggatttcgcttgtgatatttatatttgggctcctttggcggggacatgtggcgtgctgcttctgtcacttgttattacactgtactgtaaacgcgggcgaaaaaaattgctgtatatttttaagcagccatttatgaggcccgttcagacgacgcaggaggaggacggttgctcttgcaggttcccagaagaggaagaagggggctgtgaattgcgggttaaattttcaagatccgcagacgctccagcataccaacagggacaaaaccaactctataacgagctgaatcttggaagaagggaggaatatgatgtgctggataaacggcgcggtagagatccggagatgggcggaaaaccaaggcgaaaaaaccctcaggagggactctacaacgaactgcagaaagacaaaatggcggaggcttattccgaaataggcatgaagggcgagcggaggcgagggaaagggcacgacggactgtatcaaggcctctcaaccgcgactaaggatacgtacgacgccctgcacatgcaggccctgcctccgaga

SEQ ID NO: 260 - DNA, 369 bp, synthetic construct
  misc_feature 1..369: note = source = /note="Description of Artificial Sequence: Syntheticpolynucleotide"
  source 1..369: mol_type = other DNA
caggtacaattgcaggagtctggaggcggtgtggtgcaacccggtcgcagcttgcgcctgagttgtgctgcgtctggatttacattttcatcttacggaatgcattgggtacgccaggcaccggggaaaggccttgaatgggtggctgtaatttcatacgatggttccaacaaatactatgctgactcagtcaagggtcgatttacaattagtcgggacaactccaagaacaccctttatcttcaaatgaattcccttagagcagaggatacggcggtctattactgtggtggcagtggttatgcacttcatgatgattactatggcttggatgtctgggggcaagggacgcttgtaactgtatcctct

SEQ ID NO: 261 - DNA, 333 bp, synthetic construct
  misc_feature 1..333: note = source = /note="Description of Artificial Sequence: Syntheticpolynucleotide"
  source 1..333: mol_type = other DNA
caatctgctctgactcaaccagcaagcgtatcagggtcaccgggacagagtattaccataagttgcacggggacctctagcgatgtaggggggtataattatgtatcttggtatcaacaacaccccgggaaagcccctaaattgatgatctacgacgtgagcaatcgacctagtggcgtatcaaatcgcttctctggtagcaagagtgggaatacggcgtcccttactattagcggattgcaagcagaagatgaggccgattactactgcagctcctatactagctcttctacattgtacgtctttgggagcggaacaaaagtaacagtactc

SEQ ID NO: 262-274 - skipped (13 entries)

SEQ ID NO: 275 - protein, 132 aa, synthetic construct
  REGION 1..132: note = source = /note="Description of Artificial Sequence: Syntheticpolypeptide"
DVPDYASLGGPSSPKKKRKVSRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSY

SEQ ID NO: 276 - protein, 108 aa, synthetic construct
  REGION 1..108: note = source = /note="Description of Artificial Sequence: Syntheticpolypeptide"
VQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETS

SEQ ID NO: 277 - protein, 93 aa, Homo sapiens [sequence omitted]

SEQ ID NO: 278 - protein, 95 aa, synthetic construct
  REGION 1..95: note = source = /note="Description of Artificial Sequence: Syntheticpolypeptide"
ILWHEMWHEGLIEASRLYFGERNVKGMFEVLEPLHAMMERGPQTLKETSFNQAYGRDLMEAQEWCRKYMKSGNVKDLTQAWDLYYHVFRRISKTS

SEQ ID NO: 279 - protein, 95 aa, synthetic construct
  REGION 1..95: note = source = /note="Description of Artificial Sequence: Syntheticpolypeptide"
ILWHEMWHEGLLEASRLYFGERNVKGMFEVLEPLHAMMERGPQTLKETSFNQAYGRDLMEAQEWCRKYMKSGNVKDLTQAWDLYYHVFRRISKTS

SEQ ID NO: 280 - protein, 95 aa, synthetic construct
  REGION 1..95: note = source = /note="Description of Artificial Sequence: Syntheticpolypeptide"
ILWHEMWHEGLEEASRLYFGERNVKGMFEVLEPLHAMMERGPQTLKETSFNQAYGRDLMEAQEWCRKYMKSGNVKDLLQAWDLYYHVFRRISKTS

SEQ ID NO: 281 - protein, 95 aa, synthetic construct
  MOD_RES 12: note = Any amino acid
  MOD_RES 78: note = Any amino acid
  REGION 1..95: note = source = /note="Description of Artificial Sequence: Syntheticpolypeptide"
ILWHEMWHEGLXEASRLYFGERNVKGMFEVLEPLHAMMERGPQTLKETSFNQAYGRDLMEAQEWCRKYMKSGNVKDLXQAWDLYYHVFRRISKTS

SEQ ID NO: 282 - protein, 95 aa, synthetic construct
  REGION 1..95: note = source = /note="Description of Artificial Sequence: Syntheticpolypeptide"
ILWHEMWHEGLIEASRLYFGERNVKGMFEVLEPLHAMMERGPQTLKETSFNQAYGRDLMEAQEWCRKYMKSGNVKDLLQAWDLYYHVFRRISKTS

SEQ ID NO: 283 - protein, 95 aa, synthetic construct
  REGION 1..95: note = source = /note="Description of Artificial Sequence: Syntheticpolypeptide"
ILWHEMWHEGLLEASRLYFGERNVKGMFEVLEPLHAMMERGPQTLKETSFNQAYGRDLMEAQEWCRKYMKSGNVKDLLQAWDLYYHVFRRISKTS

SEQ ID NO: 284 - protein, 1132 aa, Homo sapiens [sequence omitted]

SEQ ID NO: 285 - protein, 253 aa, synthetic construct
  REGION 1..253: note = source = /note="Description of Artificial Sequence: Syntheticpolypeptide"
EVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSSGGGGSGGGGSGGGGSQSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVL

SEQ ID NO: 286 - protein, 127 aa, synthetic construct
  REGION 1..127: note = source = /note="Description of Artificial Sequence: Syntheticpolypeptide"
EVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSS

SEQ ID NO: 287 - protein, 111 aa, synthetic construct
  REGION 1..111: note = source = /note="Description of Artificial Sequence: Syntheticpolypeptide"
QSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVL

SEQ ID NO: 288 - protein, 12 aa, synthetic construct
  REGION 1..12: note = source = /note="Description of Artificial Sequence: Syntheticpeptide"
GDSMLSNSDTWN

SEQ ID NO: 289 - protein, 7 aa, synthetic construct
  REGION 1..7: note = source = /note="Description of Artificial Sequence: Syntheticpeptide"
SNSDTWN

SEQ ID NO: 290 - protein, 18 aa, synthetic construct
  REGION 1..18: note = source = /note="Description of Artificial Sequence: Syntheticpeptide"
RTYHRSTWYDDYASSVRG

SEQ ID NO: 291 - protein, 15 aa, synthetic construct
  REGION 1..15: note = source = /note="Description of Artificial Sequence: Syntheticpeptide"
VRLQDGNSWSDAFDV

SEQ ID NO: 292 - protein, 465 aa, synthetic construct
  REGION 1..465: note = source = /note="Description of Artificial Sequence: Syntheticpolypeptide"
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 293 - protein, 242 aa, synthetic construct
  REGION 1..242: note = source = /note="Description of Artificial Sequence: Syntheticpolypeptide"
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 294 - protein, 465 aa, synthetic construct
  REGION 1..465: note = source = /note="Description of Artificial Sequence: Syntheticpolypeptide"
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 295 - protein, 5 aa, synthetic construct
  REGION 1..5: note = source = /note="Description of Artificial Sequence: Syntheticpeptide"
DYGVS

SEQ ID NO: 296 - protein, 16 aa, synthetic construct
  REGION 1..16: note = source = /note="Description of Artificial Sequence: Syntheticpeptide"
VIWGSETTYYNSALKS

SEQ ID NO: 297 - protein, 12 aa, synthetic construct
  REGION 1..12: note = source = /note="Description of Artificial Sequence: Syntheticpeptide"
HYYYGGSYAMDY

SEQ ID NO: 298 - protein, 11 aa, synthetic construct
  REGION 1..11: note = source = /note="Description of Artificial Sequence: Syntheticpeptide"
RASQDISKYLN

SEQ ID NO: 299 - protein, 7 aa, synthetic construct
  REGION 1..7: note = source = /note="Description of Artificial Sequence: Syntheticpeptide"
HTSRLHS

SEQ ID NO: 300 - protein, 9 aa, synthetic construct
  REGION 1..9: note = source = /note="Description of Artificial Sequence: Syntheticpeptide"
QQGNTLPYT

SEQ ID NO: 301 - protein, 486 aa, synthetic construct
  REGION 1..486: note = source = /note="Description of Artificial Sequence: Syntheticpolypeptide"
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 302 - DNA, 1458 bp, synthetic construct
  misc_feature 1..1458: note = source = /note="Description of Artificial Sequence: Syntheticpolynucleotide"
  source 1..1458: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 303 - protein, 242 aa, synthetic construct
  REGION 1..242: note = source = /note="Description of Artificial Sequence: Syntheticpolypeptide"
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 304 - protein, 16 aa, synthetic construct
  REGION 1..16: note = source = /note="Description of Artificial Sequence: Syntheticpeptide"
VIWGSETTYYQSSLKS

SEQ ID NO: 305 - DNA, 813 bp, synthetic construct
  misc_feature 1..813: note = source = /note="Description of Artificial Sequence: Syntheticpolynucleotide"
  source 1..813: mol_type = other DNA
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagccaccaccatcatcaccatcaccat

SEQ ID NO: 306 - protein, 486 aa, synthetic construct
  REGION 1..486: note = source = /note="Description of Artificial Sequence: Syntheticpolypeptide"
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 307 - DNA, 1458 bp, synthetic construct
  misc_feature 1..1458: note = source = /note="Description of Artificial Sequence: Syntheticpolynucleotide"
  source 1..1458: mol_type = other DNA
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 308 - protein, 7 aa, synthetic construct
  REGION 1..7: note = source = /note="Description of Artificial Sequence: Syntheticpeptide"
LAEAAAK

SEQ ID NO: 309 - protein, 284 aa, synthetic construct
  REGION 1..284: note = source = /note="Description of Artificial Sequence: Syntheticpolypeptide"
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 310-330 - skipped (21 entries)

SEQ ID NO: 331 - protein, 120 aa, synthetic construct
  REGION 1..120: note = source = /note="Description of Artificial Sequence: Syntheticpolypeptide"
QVQLQESGPGLVKPSETLSLTCTVSGVSLPDYGVSWIRQPPGKCLEWIGVIWGSETTYYQSSLKSRVTISKDNSKNQVSLKLSSVTAADTAVYYCAKHYYYGGSYAMDYWGQGTLVTVSS

SEQ ID NO: 332 - protein, 107 aa, synthetic construct
  REGION 1..107: note = source = /note="Description of Artificial Sequence: Syntheticpolypeptide"
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGCGTKLEIK

SEQ ID NO: 333-348 - skipped (16 entries)

SEQ ID NO: 349 - protein, 486 aa, synthetic construct
  REGION 1..486: note = source = /note="Description of Artificial Sequence: Syntheticpolypeptide"
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 350-353 - skipped (4 entries)

SEQ ID NO: 354 - DNA, 1461 bp, synthetic construct
  misc_feature 1..1461: note = source = /note="Description of Artificial Sequence: Syntheticpolynucleotide"
  source 1..1461: mol_type = other DNA
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 355 - DNA, 1458 bp, synthetic construct
  misc_feature 1..1458: note = source = /note="Description of Artificial Sequence: Syntheticpolynucleotide"
  source 1..1458: mol_type = other DNA
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 356 - DNA, 1398 bp, synthetic construct
  misc_feature 1..1398: note = source = /note="Description of Artificial Sequence: Syntheticpolynucleotide"
  source 1..1398: mol_type = other DNA
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 357-415 - skipped (59 entries)

SEQ ID NO: 416 - DNA, 1485 bp, synthetic construct
  misc_feature 1..1485: note = source = /note="Description of Artificial Sequence: Syntheticpolynucleotide"
  source 1..1485: mol_type = other DNA
atggccctccctgtcaccgctctgttgctgccgcttgctctgctgctccacgcagcgcgaccgcaggtacaattgcaggagtctggaggcggtgtggtgcaacccggtcgcagcttgcgcctgagttgtgctgcgtctggatttacattttcatcttacggaatgcattgggtacgccaggcaccggggaaaggccttgaatgggtggctgtaatttcatacgatggttccaacaaatactatgctgactcagtcaagggtcgatttacaattagtcgggacaactccaagaacaccctttatcttcaaatgaattcccttagagcagaggatacggcggtctattactgtggtggcagtggttatgcacttcatgatgattactatggcttggatgtctgggggcaagggacgcttgtaactgtatcctctggtggtggtggtagtggtgggggaggctccggcggtggcggctctcaatctgctctgactcaaccagcaagcgtatcagggtcaccgggacagagtattaccataagttgcacggggacctctagcgatgtaggggggtataattatgtatcttggtatcaacaacaccccgggaaagcccctaaattgatgatctacgacgtgagcaatcgacctagtggcgtatcaaatcgcttctctggtagcaagagtgggaatacggcgtcccttactattagcggattgcaagcagaagatgaggccgattactactgcagctcctatactagctcttctacattgtacgtctttgggagcggaacaaaagtaacagtactcacaacaacacctgccccgagaccgcctacaccagccccgactattgccagccagcctctgagcctcaggcctgaggcctgtaggcccgcagcgggcggcgcagttcatacacggggcttggatttcgcttgtgatatttatatttgggctcctttggcggggacatgtggcgtgctgcttctgtcacttgttattacactgtactgtaaacgcgggcgaaaaaaattgctgtatatttttaagcagccatttatgaggcccgttcagacgacgcaggaggaggacggttgctcttgcaggttcccagaagaggaagaagggggctgtgaattgcgggttaaattttcaagatccgcagacgctccagcataccaacagggacaaaaccaactctataacgagctgaatcttggaagaagggaggaatatgatgtgctggataaacggcgcggtagagatccggagatgggcggaaaaccaaggcgaaaaaaccctcaggagggactctacaacgaactgcagaaagacaaaatggcggaggcttattccgaaataggcatgaagggcgagcggaggcgagggaaagggcacgacggactgtatcaaggcctctcaaccgcgactaaggatacgtacgacgccctgcacatgcaggccctgcctccgagatgataa

SEQ ID NO: 417 - DNA, 1395 bp, synthetic construct
  misc_feature 1..1395: note = source = /note="Description of Artificial Sequence: Syntheticpolynucleotide"
  source 1..1395: mol_type = other DNA
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 418 - DNA, 19 bp, synthetic construct
  misc_feature 1..19: note = source = /note="Description of Artificial Sequence: Syntheticoligonucleotide"
  source 1..19: mol_type = other DNA
gggtaagccaagaaagaaa

SEQ ID NO: 419 - DNA, 19 bp, synthetic construct
  misc_feature 1..19: note = source = /note="Description of Artificial Sequence: Syntheticoligonucleotide"
  source 1..19: mol_type = other DNA
tttctttcttggcttaccc

SEQ ID NO: 420 - protein, 10 aa, synthetic construct
  REGION 1..10: note = source = /note="Description of Artificial Sequence: Syntheticpeptide"
SASSSVSYMN

SEQ ID NO: 421 - protein, 7 aa, synthetic construct
  REGION 1..7: note = source = /note="Description of Artificial Sequence: Syntheticpeptide"
DTSKLAS

SEQ ID NO: 422 - protein, 9 aa, synthetic construct
  REGION 1..9: note = source = /note="Description of Artificial Sequence: Syntheticpeptide"
QQWSSNPFT

SEQ ID NO: 423 - protein, 6 aa, synthetic construct
  REGION 1..6: note = source = /note="Description of Artificial Sequence: Syntheticpeptide"
SSSVSY

SEQ ID NO: 424 - skipped

SEQ ID NO: 425 - protein, 6 aa, synthetic construct
  REGION 1..6: note = source = /note="Description of Artificial Sequence: Syntheticpeptide"
WSSNPF

SEQ ID NO: 426 - protein, 5 aa, synthetic construct
  REGION 1..5: note = source = /note="Description of Artificial Sequence: Syntheticpeptide"
SSVSY

SEQ ID NO: 427 - protein, 106 aa, synthetic construct [sequence omitted]

SEQ ID NO: 428 - protein, 10 aa, synthetic construct
  REGION 1..10: note = source = /note="Description of Artificial Sequence: Syntheticpeptide"
GYTFTRYTMH

SEQ ID NO: 429 - protein, 17 aa, synthetic construct
  REGION 1..17: note = source = /note="Description of Artificial Sequence: Syntheticpeptide"
YINPSRGYTNYNQKVKD

SEQ ID NO: 430 - protein, 10 aa, synthetic construct
  REGION 1..10: note = source = /note="Description of Artificial Sequence: Syntheticpeptide"
YYDDHYSLDY

SEQ ID NO: 431 - protein, 5 aa, synthetic construct
  REGION 1..5: note = source = /note="Description of Artificial Sequence: Syntheticpeptide"
RYTMH

SEQ ID NO: 432 - protein, 7 aa, synthetic construct
  REGION 1..7: note = source = /note="Description of Artificial Sequence: Syntheticpeptide"
GYTFTRY

SEQ ID NO: 433 - protein, 6 aa, synthetic construct
  REGION 1..6: note = source = /note="Description of Artificial Sequence: Syntheticpeptide"
NPSRGY

SEQ ID NO: 434 - protein, 8 aa, synthetic construct
  REGION 1..8: note = source = /note="Description of Artificial Sequence: Syntheticpeptide"
GYTFTRYT

SEQ ID NO: 435 - protein, 8 aa, synthetic construct
  REGION 1..8: note = source = /note="Description of Artificial Sequence: Syntheticpeptide"
INPSRGYT

SEQ ID NO: 436 - protein, 12 aa, synthetic construct
  REGION 1..12: note = source = /note="Description of Artificial Sequence: Syntheticpeptide"
ARYYDDHYSLDY

SEQ ID NO: 437 - protein, 119 aa, synthetic construct
  REGION 1..119: note = source = /note="Description of Artificial Sequence: Syntheticpolypeptide"
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSS

SEQ ID NO: 438 - protein, 14 aa, synthetic construct
  REGION 1..14: note = source = /note="Description of Artificial Sequence: Syntheticpeptide"
GSSTGAVTSGNYPN

SEQ ID NO: 439 - protein, 7 aa, synthetic construct
  REGION 1..7: note = source = /note="Description of Artificial Sequence: Syntheticpeptide"
GTKFLAP

SEQ ID NO: 440 - protein, 9 aa, synthetic construct
  REGION 1..9: note = source = /note="Description of Artificial Sequence: Syntheticpeptide"
VLWYSNRWV

SEQ ID NO: 441 - protein, 10 aa, synthetic construct
  REGION 1..10: note = source = /note="Description of Artificial Sequence: Syntheticpeptide"
STGAVTSGNY

SEQ ID NO: 442 - skipped

SEQ ID NO: 443 - protein, 6 aa, synthetic construct
  REGION 1..6: note = source = /note="Description of Artificial Sequence: Syntheticpeptide"
WYSNRW

SEQ ID NO: 444 - protein, 9 aa, synthetic construct
  REGION 1..9: note = source = /note="Description of Artificial Sequence: Syntheticpeptide"
TGAVTSGNY

SEQ ID NO: 445 - protein, 109 aa, synthetic construct
  REGION 1..109: note = source = /note="Description of Artificial Sequence: Syntheticpolypeptide"
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 446 - protein, 109 aa, synthetic construct
  REGION 1..109: note = source = /note="Description of Artificial Sequence: Syntheticpolypeptide"
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGCGTKLTVL

SEQ ID NO: 447 - protein, 10 aa, synthetic construct
  REGION 1..10: note = source = /note="Description of Artificial Sequence: Syntheticpeptide"
GFTFNKYAMN

SEQ ID NO: 448 - protein, 19 aa, synthetic construct
  REGION 1..19: note = source = /note="Description of Artificial Sequence: Syntheticpeptide"
RIRSKYNNYATYYADSVKD

SEQ ID NO: 449 - protein, 14 aa, synthetic construct
  REGION 1..14: note = source = /note="Description of Artificial Sequence: Syntheticpeptide"
HGNFGNSYISYWAY

SEQ ID NO: 450 - protein, 5 aa, synthetic construct
  REGION 1..5: note = source = /note="Description of Artificial Sequence: Syntheticpeptide"
KYAMN

SEQ ID NO: 451 - protein, 7 aa, synthetic construct
  REGION 1..7: note = source = /note="Description of Artificial Sequence: Syntheticpeptide"
GFTFNKY

SEQ ID NO: 452 - protein, 8 aa, synthetic construct
  REGION 1..8: note = source = /note="Description of Artificial Sequence: Syntheticpeptide"
RSKYNNYA

SEQ ID NO: 453 - protein, 8 aa, synthetic construct
  REGION 1..8: note = source = /note="Description of Artificial Sequence: Syntheticpeptide"
GFTFNKYA

SEQ ID NO: 454 - protein, 10 aa, synthetic construct
  REGION 1..10: note = source = /note="Description of Artificial Sequence: Syntheticpeptide"
IRSKYNNYAT

SEQ ID NO: 455 - protein, 16 aa, synthetic construct
  REGION 1..16: note = source = /note="Description of Artificial Sequence: Syntheticpeptide"
VRHGNFGNSYISYWAY

SEQ ID NO: 456 - protein, 125 aa, synthetic construct
  REGION 1..125: note = source = /note="Description of Artificial Sequence: Syntheticpolypeptide"
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 457 - protein, 125 aa, synthetic construct
  REGION 1..125: note = source = /note="Description of Artificial Sequence: Syntheticpolypeptide"
EVQLVESGGGLVQPGGSLKLSCAASGFTFNKYAMNWVRQAPGKCLEWVARIRSKYNNYATYYADSVKDRFTISRDDSKNTAYLQMNNLKTEDTAVYYCVRHGNFGNSYISYWAYWGQGTLVTVSS

SEQ ID NO: 458 - protein, 14 aa, synthetic construct
  REGION 1..14: note = source = /note="Description of Artificial Sequence: Syntheticpeptide"
RSSTGAVTTSNYAN

SEQ ID NO: 459 - protein, 7 aa, synthetic construct
  REGION 1..7: note = source = /note="Description of Artificial Sequence: Syntheticpeptide"
GTNKRAP

SEQ ID NO: 460 - protein, 9 aa, synthetic construct
  REGION 1..9: note = source = /note="Description of Artificial Sequence: Syntheticpeptide"
ALWYSNLWV

SEQ ID NO: 461 - protein, 10 aa, synthetic construct
  REGION 1..10: note = source = /note="Description of Artificial Sequence: Syntheticpeptide"
STGAVTTSNY

SEQ ID NO: 462 - skipped

SEQ ID NO: 463 - protein, 6 aa, synthetic construct
  REGION 1..6: note = source = /note="Description of Artificial Sequence: Syntheticpeptide"
WYSNLW

SEQ ID NO: 464 - protein, 9 aa, synthetic construct
  REGION 1..9: note = source = /note="Description of Artificial Sequence: Syntheticpeptide"
TGAVTTSNY

SEQ ID NO: 465 - skipped

SEQ ID NO: 466 - protein, 7 aa, synthetic construct
  REGION 1..7: note = source = /note="Description of Artificial Sequence: Syntheticpeptide"
ALWYSNL

SEQ ID NO: 467 - protein, 109 aa, synthetic construct
  REGION 1..109: note = source = /note="Description of Artificial Sequence: Syntheticpolypeptide"
QAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVL

SEQ ID NO: 468 - protein, 109 aa, synthetic construct
  REGION 1..109: note = source = /note="Description of Artificial Sequence: Syntheticpolypeptide"
QAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGCGTKLTVL

SEQ ID NO: 469 - protein, 10 aa, synthetic construct
  REGION 1..10: note = source = /note="Description of Artificial Sequence: Syntheticpeptide"
GFTFNTYAMN

SEQ ID NO: 470 - protein, 14 aa, synthetic construct
  REGION 1..14: note = source = /note="Description of Artificial Sequence: Syntheticpeptide"
HGNFGNSYVSWFAY

SEQ ID NO: 471 - protein, 5 aa, synthetic construct
  REGION 1..5: note = source = /note="Description of Artificial Sequence: Syntheticpeptide"
TYAMN

SEQ ID NO: 472 - protein, 7 aa, synthetic construct
  REGION 1..7: note = source = /note="Description of Artificial Sequence: Syntheticpeptide"
GFTFNTY

SEQ ID NO: 473 - protein, 8 aa, synthetic construct
  REGION 1..8: note = source = /note="Description of Artificial Sequence: Syntheticpeptide"
GFTFNTYA

SEQ ID NO: 474 - protein, 16 aa, synthetic construct
  REGION 1..16: note = source = /note="Description of Artificial Sequence: Syntheticpeptide"
VRHGNFGNSYVSWFAY

SEQ ID NO: 475 - protein, 125 aa, synthetic construct
  REGION 1..125: note = source = /note="Description of Artificial Sequence: Syntheticpolypeptide"
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSS

SEQ ID NO: 476 - protein, 125 aa, synthetic construct
  REGION 1..125: note = source = /note="Description of Artificial Sequence: Syntheticpolypeptide"
EVQLVESGGGLVQPGGSLKLSCAASGFTFNTYAMNWVRQASGKCLEWVGRIRSKYNNYATYYADSVKDRFTISRDDSKSTLYLQMNSLKTEDTAVYYCVRHGNFGNSYVSWFAYWGQGTLVTVSS

SEQ ID NO: 477 - protein, 16 aa, synthetic construct
  REGION 1..16: note = source = /note="Description of Artificial Sequence: Syntheticpeptide"
RSSQSLVRSEGTTYFN

SEQ ID NO: 478 - protein, 7 aa, synthetic construct
  REGION 1..7: note = source = /note="Description of Artificial Sequence: Syntheticpeptide"
RVSNRFS

SEQ ID NO: 479 - protein, 9 aa, synthetic construct
  REGION 1..9: note = source = /note="Description of Artificial Sequence: Syntheticpeptide"
LQSSHFPWT

SEQ ID NO: 480 - protein, 12 aa, synthetic construct
  REGION 1..12: note = source = /note="Description of Artificial Sequence: Syntheticpeptide"
SQSLVRSEGTTY

SEQ ID NO: 481 - skipped

SEQ ID NO: 482 - protein, 6 aa, synthetic construct
  REGION 1..6: note = source = /note="Description of Artificial Sequence: Syntheticpeptide"
SSHFPW

SEQ ID NO: 483 - protein, 11 aa, synthetic construct
  REGION 1..11: note = source = /note="Description of Artificial Sequence: Syntheticpeptide"
QSLVRSEGTTY

SEQ ID NO: 484 - protein, 112 aa, synthetic construct
  REGION 1..112: note = source = /note="Description of Artificial Sequence: Syntheticpolypeptide"
DILVTQTPVSLPVSLGGHVSISCRSSQSLVRSEGTTYFNWYLQKPGQSPQLLIYRVSNRFSGVPDRFSGSGSGTDFTLKISRVEPEDLGVYYCLQSSHFPWTFGGGTKLELK

SEQ ID NO: 485 - protein, 10 aa, synthetic construct
  REGION 1..10: note = source = /note="Description of Artificial Sequence: Syntheticpeptide"
GFTFSKQGMH

SEQ ID NO: 486 - protein, 17 aa, synthetic construct
  REGION 1..17: note = source = /note="Description of Artificial Sequence: Syntheticpeptide"
MIYYDSSKMYYADTVKG

SEQ ID NO: 487 - protein, 9 aa, synthetic construct
  REGION 1..9: note = source = /note="Description of Artificial Sequence: Syntheticpeptide"
FWWDLDFDH

SEQ ID NO: 488 - protein, 5 aa, synthetic construct
  REGION 1..5: note = source = /note="Description of Artificial Sequence: Syntheticpeptide"
KQGMH

SEQ ID NO: 489 - protein, 7 aa, synthetic construct
  REGION 1..7: note = source = /note="Description of Artificial Sequence: Syntheticpeptide"
GFTFSKQ

SEQ ID NO: 490 - protein, 6 aa, synthetic construct
  REGION 1..6: note = source = /note="Description of Artificial Sequence: Syntheticpeptide"
YYDSSK

SEQ ID NO: 491 - protein, 8 aa, synthetic construct
  REGION 1..8: note = source = /note="Description of Artificial Sequence: Syntheticpeptide"
GFTFSKQG

SEQ ID NO: 492 - protein, 8 aa, synthetic construct
  REGION 1..8: note = source = /note="Description of Artificial Sequence: Syntheticpeptide"
IYYDSSKM

SEQ ID NO: 493 - protein, 11 aa, synthetic construct
  REGION 1..11: note = source = /note="Description of Artificial Sequence: Syntheticpeptide"
ASFWWDLDFDH

SEQ ID NO: 494 - protein, 118 aa, synthetic construct
  REGION 1..118: note = source = /note="Description of Artificial Sequence: Syntheticpolypeptide"
EVKLVESGGDLVQPGDSLTLSCVASGFTFSKQGMHWIRQAPKKGLEWIAMIYYDSSKMYYADTVKGRFTISRDNSKNTLYLEMNSLRSEDTAMYYCASFWWDLDFDHWGQGVMVTVSS

SEQ ID NO: 495 - protein, 10 aa, synthetic construct
  REGION 1..10: note = source = /note="Description of Artificial Sequence: Syntheticpeptide"
SATSSVSYMH

SEQ ID NO: 496 - protein, 9 aa, synthetic construct
  REGION 1..9: note = source = /note="Description of Artificial Sequence: Syntheticpeptide"
QQWSSNPLT

SEQ ID NO: 497 - protein, 6 aa, synthetic construct
  REGION 1..6: note = source = /note="Description of Artificial Sequence: Syntheticpeptide"
TSSVSY

SEQ ID NO: 498 - protein, 6 aa, synthetic construct
  REGION 1..6: note = source = /note="Description of Artificial Sequence: Syntheticpeptide"
WSSNPL

SEQ ID NO: 499 - protein, 106 aa, synthetic construct
  REGION 1..106: note = source = /note="Description of Artificial Sequence: Syntheticpolypeptide"
QIVLTQSPAIMSASPGEKVTMTCSATSSVSYMHWYQQKSGTSPKRWIYDTSKLASGVPARFSGSGSGTSYSLTISSMEAEDAATYYCQQWSSNPLTFGAGTKLELK

SEQ ID NO: 500 - protein, 10 aa, synthetic construct
  REGION 1..10: note = source = /note="Description of Artificial Sequence: Syntheticpeptide"
GYKFTSYVMH

SEQ ID NO: 501 - protein, 17 aa, synthetic construct
  REGION 1..17: note = source = /note="Description of Artificial Sequence: Syntheticpeptide"
YINPYNDVTKYNEKFKG

SEQ ID NO: 502 - protein, 11 aa, synthetic construct
  REGION 1..11: note = source = /note="Description of Artificial Sequence: Syntheticpeptide"
GSYYDYDGFVY

SEQ ID NO: 503 - protein, 5 aa, synthetic construct
  REGION 1..5: note = source = /note="Description of Artificial Sequence: Syntheticpeptide"
SYVMH

SEQ ID NO: 504 - protein, 7 aa, synthetic construct
  REGION 1..7: note = source = /note="Description of Artificial Sequence: Syntheticpeptide"
GYKFTSY

SEQ ID NO: 505 - protein, 6 aa, synthetic construct
  REGION 1..6: note = source = /note="Description of Artificial Sequence: Syntheticpeptide"
NPYNDV

SEQ ID NO: 506 - protein, 8 aa, synthetic construct
  REGION 1..8: note = source = /note="Description of Artificial Sequence: Syntheticpeptide"
GYKFTSYV

SEQ ID NO: 507 - protein, 8 aa, synthetic construct
  REGION 1..8: note = source = /note="Description of Artificial Sequence: Syntheticpeptide"
INPYNDVT

SEQ ID NO: 508 - protein, 13 aa, synthetic construct
  REGION 1..13: note = source = /note="Description of Artificial Sequence: Syntheticpeptide"
ARGSYYDYDGFVY

SEQ ID NO: 509 - protein, 120 aa, synthetic construct
  REGION 1..120: note = source = /note="Description of Artificial Sequence: Syntheticpolypeptide"
EVQLQQSGPELVKPGASVKMSCKASGYKFTSYVMHWVKQKPGQGLEWIGYINPYNDVTKYNEKFKGKATLTSDKSSSTAYMELSSLTSEDSAVHYCARGSYYDYDGFVYWGQGTLVTVSA

SEQ ID NO: 510 - protein, 11 aa, synthetic construct
  REGION 1..11: note = source = /note="Description of Artificial Sequence: Syntheticpeptide"
HASQNIYVWLN

SEQ ID NO: 511 - protein, 7 aa, synthetic construct
  REGION 1..7: note = source = /note="Description of Artificial Sequence: Syntheticpeptide"
KASNLHT

SEQ ID NO: 512 - protein, 9 aa, synthetic construct
  REGION 1..9: note = source = /note="Description of Artificial Sequence: Syntheticpeptide"
QQGQTYPYT

SEQ ID NO: 513 - protein, 7 aa, synthetic construct
  REGION 1..7: note = source = /note="Description of Artificial Sequence: Syntheticpeptide"
SQNIYVW

SEQ ID NO: 514 - skipped

SEQ ID NO: 515 - protein, 6 aa, synthetic construct
  REGION 1..6: note = source = /note="Description of Artificial Sequence: Syntheticpeptide"
GQTYPY

SEQ ID NO: 516 - protein, 6 aa, synthetic construct
  REGION 1..6: note = source = /note="Description of Artificial Sequence: Syntheticpeptide"
QNIYVW

SEQ ID NO: 517 - protein, 107 aa, synthetic construct
  REGION 1..107: note = source = /note="Description of Artificial Sequence: Syntheticpolypeptide"
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 518 - protein, 214 aa, synthetic construct
  REGION 1..214: note = source = /note="Description of Artificial Sequence: Syntheticpolypeptide"
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 519 - protein, 10 aa, synthetic construct
  REGION 1..10: note = source = /note="Description of Artificial Sequence: Syntheticpeptide"
GYTFTSYYIH

SEQ ID NO: 520 - protein, 17 aa, synthetic construct
  REGION 1..17: note = source = /note="Description of Artificial Sequence: Syntheticpeptide"
CIYPGNVNTNYNEKFKD

SEQ ID NO: 521 - protein, 11 aa, synthetic construct
  REGION 1..11: note = source = /note="Description of Artificial Sequence: Syntheticpeptide"
SHYGLDWNFDV

SEQ ID NO: 522 - protein, 5 aa, synthetic construct
  REGION 1..5: note = source = /note="Description of Artificial Sequence: Syntheticpeptide"
SYYIH

SEQ ID NO: 523 - protein, 7 aa, synthetic construct
  REGION 1..7: note = source = /note="Description of Artificial Sequence: Syntheticpeptide"
GYTFTSY

SEQ ID NO: 524 - protein, 6 aa, synthetic construct
  REGION 1..6: note = source = /note="Description of Artificial Sequence: Syntheticpeptide"
YPGNVN

SEQ ID NO: 525 - protein, 8 aa, synthetic construct
  REGION 1..8: note = source = /note="Description of Artificial Sequence: Syntheticpeptide"
GYTFTSYY

SEQ ID NO: 526 - protein, 8 aa, synthetic construct
  REGION 1..8: note = source = /note="Description of Artificial Sequence: Syntheticpeptide"
IYPGNVNT

SEQ ID NO: 527 - protein, 13 aa, synthetic construct
  REGION 1..13: note = source = /note="Description of Artificial Sequence: Syntheticpeptide"
TRSHYGLDWNFDV

SEQ ID NO: 528 - protein, 120 aa, synthetic construct
  REGION 1..120: note = source = /note="Description of Artificial Sequence: Syntheticpolypeptide"
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS

SEQ ID NO: 529 - protein, 223 aa, synthetic construct
  REGION 1..223: note = source = /note="Description of Artificial Sequence: Syntheticpolypeptide"
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 530 - protein, 13 aa, synthetic construct
  REGION 1..13: note = source = /note="Description of Artificial Sequence: Syntheticpeptide"
SGSSSNIVSNYVN

SEQ ID NO: 531 - protein, 7 aa, synthetic construct
  REGION 1..7: note = source = /note="Description of Artificial Sequence: Syntheticpeptide"
DNNKRPS

SEQ ID NO: 532 - protein, 11 aa, synthetic construct
  REGION 1..11: note = source = /note="Description of Artificial Sequence: Syntheticpeptide"
QSYAIGSYSVV

SEQ ID NO: 533 - protein, 9 aa, synthetic construct
  REGION 1..9: note = source = /note="Description of Artificial Sequence: Syntheticpeptide"
SSSNIVSNY

SEQ ID NO: 534 - skipped

SEQ ID NO: 535 - protein, 8 aa, synthetic construct
  REGION 1..8: note = source = /note="Description of Artificial Sequence: Syntheticpeptide"
YAIGSYSV

SEQ ID NO: 536 - protein, 8 aa, synthetic construct
  REGION 1..8: note = source = /note="Description of Artificial Sequence: Syntheticpeptide"
SSNIVSNY

SEQ ID NO: 537 - protein, 110 aa, synthetic construct
  REGION 1..110: note = source = /note="Description of Artificial Sequence: Syntheticpolypeptide"
DIVLTQPPSVSGAPGQRVTISCSGSSSNIVSNYVNWYQQLPGTAPKLLIYDNNKRPSGVPDRFSGSKSGTSASLAITGLQSEDEADYYCQSYAIGSYSVVFGGGTKLTVL

SEQ ID NO: 538 - protein, 10 aa, synthetic construct
  REGION 1..10: note = source = /note="Description of Artificial Sequence: Syntheticpeptide"
GFTFSTYGMS

SEQ ID NO: 539 - protein, 17 aa, synthetic construct
  REGION 1..17: note = source = /note="Description of Artificial Sequence: Syntheticpeptide"
SIFYTGSSTYYADSVKG

SEQ ID NO: 540 - protein, 11 aa, synthetic construct
  REGION 1..11: note = source = /note="Description of Artificial Sequence: Syntheticpeptide"
IGYAGDSKYAI

SEQ ID NO: 541 - protein, 5 aa, synthetic construct
  REGION 1..5: note = source = /note="Description of Artificial Sequence: Syntheticpeptide"
TYGMS

SEQ ID NO: 542 - protein, 7 aa, synthetic construct
  REGION 1..7: note = source = /note="Description of Artificial Sequence: Syntheticpeptide"
GFTFSTY

SEQ ID NO: 543 - protein, 6 aa, synthetic construct
  REGION 1..6: note = source = /note="Description of Artificial Sequence: Syntheticpeptide"
FYTGSS

SEQ ID NO: 544 - protein, 8 aa, synthetic construct
  REGION 1..8: note = source = /note="Description of Artificial Sequence: Syntheticpeptide"
GFTFSTYG

SEQ ID NO: 545 - protein, 8 aa, synthetic construct
  REGION 1..8: note = source = /note="Description of Artificial Sequence: Syntheticpeptide"
IFYTGSST

SEQ ID NO: 546 - protein, 13 aa, synthetic construct
  REGION 1..13: note = source = /note="Description of Artificial Sequence: Syntheticpeptide"
ARIGYAGDSKYAI

SEQ ID NO: 547 - protein, 120 aa, synthetic construct
  REGION 1..120: note = source = /note="Description of Artificial Sequence: Syntheticpolypeptide"
QVQLVESGGGLVQPGGSLRLSCAASGFTFSTYGMSWVRQAPGKGLEWVSSIFYTGSSTYYADSVKGRFTISRDNSKNTLYLQMNSLRAEDTAVYYCARIGYAGDSKYAIWGQGTLVTVSS

SEQ ID NO: 548 - protein, 120 aa, synthetic construct
  REGION 1..120: note = source = /note="Description of Artificial Sequence: Syntheticpolypeptide"
EVQLVESGGGLVQPGGSLRLSCAASGFTFSTYGMSWVRQAPGKGLEWVSSIFYTGSSTYYADSVKGRFTISRDNSKNTLYLQMNSLRAEDTAVYYCARIGYAGDSKYAIWGQGTLVTVSS

SEQ ID NO: 549 - protein, 15 aa, synthetic construct
  REGION 1..15: note = source = /note="Description of Artificial Sequence: Syntheticpeptide"
KSSQSLLSGSFNYLT

SEQ ID NO: 550 - protein, 7 aa, synthetic construct
  REGION 1..7: note = source = /note="Description of Artificial Sequence: Syntheticpeptide"
YASTRHT

SEQ ID NO: 551 - protein, 9 aa, synthetic construct
  REGION 1..9: note = source = /note="Description of Artificial Sequence: Syntheticpeptide"
HHHYNAPPT

SEQ ID NO: 552 - protein, 11 aa, synthetic construct
  REGION 1..11: note = source = /note="Description of Artificial Sequence: Syntheticpeptide"
SQSLLSGSFNY

SEQ ID NO: 553 - skipped

SEQ ID NO: 554 - protein, 6 aa, synthetic construct
  REGION 1..6: note = source = /note="Description of Artificial Sequence: Syntheticpeptide"
HYNAPP

SEQ ID NO: 555 - protein, 10 aa, synthetic construct
  REGION 1..10: note = source = /note="Description of Artificial Sequence: Syntheticpeptide"
QSLLSGSFNY

SEQ ID NO: 556 - protein, 111 aa, synthetic construct
  REGION 1..111: note = source = /note="Description of Artificial Sequence: Syntheticpolypeptide"
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIK

SEQ ID NO: 557 - protein, 218 aa, synthetic construct
  REGION 1..218: note = source = /note="Description of Artificial Sequence: Syntheticpolypeptide"
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 558 - protein, 10 aa, synthetic construct
  REGION 1..10: note = source = /note="Description of Artificial Sequence: Syntheticpeptide"
GFTFSDYWMD

SEQ ID NO: 559 - protein, 17 aa, synthetic construct
  REGION 1..17: note = source = /note="Description of Artificial Sequence: Syntheticpeptide"
NIDEDGSITEYSPFVKG

SEQ ID NO: 560 - protein, 8 aa, synthetic construct
  REGION 1..8: note = source = /note="Description of Artificial Sequence: Syntheticpeptide"
WGRFGFDS

SEQ ID NO: 561 - protein, 5 aa, synthetic construct
  REGION 1..5: note = source = /note="Description of Artificial Sequence: Syntheticpeptide"
DYWMD

SEQ ID NO: 562 - protein, 7 aa, synthetic construct
  REGION 1..7: note = source = /note="Description of Artificial Sequence: Syntheticpeptide"
GFTFSDY

SEQ ID NO: 563 - protein, 6 aa, synthetic construct
  REGION 1..6: note = source = /note="Description of Artificial Sequence: Syntheticpeptide"
DEDGSI

SEQ ID NO: 564 - protein, 8 aa, synthetic construct
  REGION 1..8: note = source = /note="Description of Artificial Sequence: Syntheticpeptide"
GFTFSDYW

SEQ ID NO: 565 - protein, 8 aa, synthetic construct
  REGION 1..8: note = source = /note="Description of Artificial Sequence: Syntheticpeptide"
IDEDGSIT

SEQ ID NO: 566 - protein, 10 aa, synthetic construct
  REGION 1..10: note = source = /note="Description of Artificial Sequence: Syntheticpeptide"
TRWGRFGFDS

SEQ ID NO: 567 - protein, 117 aa, synthetic construct
  REGION 1..117: note = source = /note="Description of Artificial Sequence: Syntheticpolypeptide"
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSS

SEQ ID NO: 568 - protein, 11 aa, synthetic construct
  REGION 1..11: note = source = /note="Description of Artificial Sequence: Syntheticpeptide"
RASQGISRWLA

SEQ ID NO: 569 - protein, 9 aa, synthetic construct
  REGION 1..9: note = source = /note="Description of Artificial Sequence: Syntheticpeptide"
QQYNTYPRT

SEQ ID NO: 570 - protein, 7 aa, synthetic construct
  REGION 1..7: note = source = /note="Description of Artificial Sequence: Syntheticpeptide"
SQGISRW

SEQ ID NO: 571 - protein, 6 aa, synthetic construct
  REGION 1..6: note = source = /note="Description of Artificial Sequence: Syntheticpeptide"
YNTYPR

SEQ ID NO: 572 - protein, 6 aa, synthetic construct
  REGION 1..6: note = source = /note="Description of Artificial Sequence: Syntheticpeptide"
QGISRW

SEQ ID NO: 573 - protein, 107 aa, synthetic construct
  REGION 1..107: note = source = /note="Description of Artificial Sequence: Syntheticpolypeptide"
DIQMTQSPSSLSASVGDRVTITCRASQGISRWLAWYQQKPEKAPKSLIYAASSLQSGVPSRFSGSGSGTDFTLTISSLQPEDFATYYCQQYNTYPRTFGQGTKVEIK

SEQ ID NO: 574 - protein, 10 aa, synthetic construct
  REGION 1..10: note = source = /note="Description of Artificial Sequence: Syntheticpeptide"
GFTFSSYDMH

SEQ ID NO: 575 - protein, 17 aa, synthetic construct
  REGION 1..17: note = source = /note="Description of Artificial Sequence: Syntheticpeptide"
VIWYDGSNKYYADSVKG

SEQ ID NO: 576 - protein, 10 aa, synthetic construct
  REGION 1..10: note = source = /note="Description of Artificial Sequence: Syntheticpeptide"
GSGNWGFFDY

SEQ ID NO: 577 - protein, 5 aa, synthetic construct
  REGION 1..5: note = source = /note="Description of Artificial Sequence: Syntheticpeptide"
SYDMH

SEQ ID NO: 578 - protein, 6 aa, synthetic construct
  REGION 1..6: note = source = /note="Description of Artificial Sequence: Syntheticpeptide"
WYDGSN

SEQ ID NO: 579 - protein, 8 aa, synthetic construct
  REGION 1..8: note = source = /note="Description of Artificial Sequence: Syntheticpeptide"
GFTFSSYD

SEQ ID NO: 580 - protein, 8 aa, synthetic construct
  REGION 1..8: note = source = /note="Description of Artificial Sequence: Syntheticpeptide"
IWYDGSNK

SEQ ID NO: 581 - protein, 12 aa, synthetic construct
  REGION 1..12: note = source = /note="Description of Artificial Sequence: Syntheticpeptide"
ARGSGNWGFFDY

SEQ ID NO: 582 - protein, 118 aa, synthetic construct
  REGION 1..118: note = source = /note="Description of Artificial Sequence: Syntheticpolypeptide"
QVQLVESGGGVVQPGRSLRLSCAASGFTFSSYDMHWVRQAPGGLEWVAVIWYDGSNKYYADSVKGRFTISRDNSKNTLYLQMNSLRAEDTAVYYCARGSGNWGFFDYWGQGTLVTVSS

SEQ ID NO: 583 - protein, 10 aa, synthetic construct
  REGION 1..10: note = source = /note="Description of Artificial Sequence: Syntheticpeptide"
SASSSRSYMQ

SEQ ID NO: 584 - protein, 7 aa, synthetic construct
  REGION 1..7: note = source = /note="Description of Artificial Sequence: Syntheticpeptide"
HQRSSYT

SEQ ID NO: 585 - protein, 6 aa, synthetic construct
  REGION 1..6: note = source = /note="Description of Artificial Sequence: Syntheticpeptide"
SSSRSY

SEQ ID NO: 586 - protein, 4 aa, synthetic construct
  REGION 1..4: note = source = /note="Description of Artificial Sequence: Syntheticpeptide"
RSSY

SEQ ID NO: 587 - protein, 5 aa, synthetic construct
  REGION 1..5: note = source = /note="Description of Artificial Sequence: Syntheticpeptide"
SSRSY

SEQ ID NO: 588 - protein, 104 aa, synthetic construct [sequence omitted]

SEQ ID NO: 589 - protein, 10 aa, synthetic construct
  REGION 1..10: note = source = /note="Description of Artificial Sequence: Syntheticpeptide"
GYSFTRYWMH

SEQ ID NO: 590 - protein, 17 aa, synthetic construct
  REGION 1..17: note = source = /note="Description of Artificial Sequence: Syntheticpeptide"
AIYPGNSDTSYNQKFEG

SEQ ID NO: 591 - protein, 8 aa, synthetic construct
  REGION 1..8: note = source = /note="Description of Artificial Sequence: Syntheticpeptide"
DYGYYFDF

SEQ ID NO: 592 - protein, 5 aa, synthetic construct
  REGION 1..5: note = source = /note="Description of Artificial Sequence: Syntheticpeptide"
RYWMH

SEQ ID NO: 593 - protein, 7 aa, synthetic construct
  REGION 1..7: note = source = /note="Description of Artificial Sequence: Syntheticpeptide"
GYSFTRY

SEQ ID NO: 594 - protein, 6 aa, synthetic construct
  REGION 1..6: note = source = /note="Description of Artificial Sequence: Syntheticpeptide"
YPGNSD

SEQ ID NO: 595 - protein, 8 aa, synthetic construct
  REGION 1..8: note = source = /note="Description of Artificial Sequence: Syntheticpeptide"
GYSFTRYW

SEQ ID NO: 596 - protein, 8 aa, synthetic construct
  REGION 1..8: note = source = /note="Description of Artificial Sequence: Syntheticpeptide"
IYPGNSDT

SEQ ID NO: 597 - protein, 10 aa, synthetic construct
  REGION 1..10: note = source = /note="Description of Artificial Sequence: Syntheticpeptide"
SRDYGYYFDF

SEQ ID NO: 598 - protein, 117 aa, synthetic construct
  REGION 1..117: note = source = /note="Description of Artificial Sequence: Syntheticpolypeptide"
EVQLQQSGTVLARPGASVKMSCKASGYSFTRYWMHWIKQRPGQGLEWIGAIYPGNSDTSYNQKFEGKAKLTAVTSASTAYMELSSLTHEDSAVYYCSRDYGYYFDFWGQGTTLTVSS

SEQ ID NO: 599 - protein, 15 aa, synthetic construct
  REGION 1..15: note = source = /note="Description of Artificial Sequence: Syntheticpeptide"
KASQSVDYDGDSYMN

SEQ ID NO: 600 - protein, 7 aa, synthetic construct
  REGION 1..7: note = source = /note="Description of Artificial Sequence: Syntheticpeptide"
AASNLES

SEQ ID NO: 601 - protein, 9 aa, synthetic construct
  REGION 1..9: note = source = /note="Description of Artificial Sequence: Syntheticpeptide"
QQSNEDPYT

SEQ ID NO: 602 - protein, 11 aa, synthetic construct
  REGION 1..11: note = source = /note="Description of Artificial Sequence: Syntheticpeptide"
SQSVDYDGDSY

SEQ ID NO: 603 - protein, 6 aa, synthetic construct
  REGION 1..6: note = source = /note="Description of Artificial Sequence: Syntheticpeptide"
SNEDPY

SEQ ID NO: 604 - protein, 10 aa, synthetic construct
  REGION 1..10: note = source = /note="Description of Artificial Sequence: Syntheticpeptide"
QSVDYDGDSY

SEQ ID NO: 605 - protein, 111 aa, synthetic construct
  REGION 1..111: note = source = /note="Description of Artificial Sequence: Syntheticpolypeptide"
DIVLTQSPLSLPVTLGQPASISCKASQSVDYDGDSYMNWYQQRPGQSPRLLIYAASNLESGVPDRFSGSGSGTDFTLKISRVEAEDVGVYYCQQSNEDPYTFGGGTKVEIK

SEQ ID NO: 606 - protein, 218 aa, synthetic construct
  REGION 1..218: note = source = /note="Description of Artificial Sequence: Syntheticpolypeptide"
DIVLTQSPLSLPVTLGQPASISCKASQSVDYDGDSYMNWYQQRPGQSPRLLIYAASNLESGVPDRFSGSGSGTDFTLKISRVEAEDVGVYYCQQSNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 607 - protein, 10 aa, synthetic construct
  REGION 1..10: note = source = /note="Description of Artificial Sequence: Syntheticpeptide"
GYAFTNYLIE

SEQ ID NO: 608 - protein, 17 aa, synthetic construct
  REGION 1..17: note = source = /note="Description of Artificial Sequence: Syntheticpeptide"
VINPGSGGTNYNEKFKG

SEQ ID NO: 609 - protein, 12 aa, synthetic construct
  REGION 1..12: note = source = /note="Description of Artificial Sequence: Syntheticpeptide"
WRGDGYYAYFDV

SEQ ID NO: 610 - protein, 5 aa, synthetic construct
  REGION 1..5: note = source = /note="Description of Artificial Sequence: Syntheticpeptide"
NYLIE

SEQ ID NO: 611 - protein, 7 aa, synthetic construct
  REGION 1..7: note = source = /note="Description of Artificial Sequence: Syntheticpeptide"
GYAFTNY

SEQ ID NO: 612 - protein, 6 aa, synthetic construct
  REGION 1..6: note = source = /note="Description of Artificial Sequence: Syntheticpeptide"
NPGSGG

SEQ ID NO: 613 - protein, 8 aa, synthetic construct
  REGION 1..8: note = source = /note="Description of Artificial Sequence: Syntheticpeptide"
GYAFTNYL

SEQ ID NO: 614 - protein, 8 aa, synthetic construct
  REGION 1..8: note = source = /note="Description of Artificial Sequence: Syntheticpeptide"
INPGSGGT

SEQ ID NO: 615 - protein, 14 aa, synthetic construct
  REGION 1..14: note = source = /note="Description of Artificial Sequence: Syntheticpeptide"
ARWRGDGYYAYFDV

SEQ ID NO: 616 - protein, 121 aa, synthetic construct
  REGION 1..121: note = source = /note="Description of Artificial Sequence: Syntheticpolypeptide"
EVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGDGYYAYFDVWGQGTTVTVSS

SEQ ID NO: 617 - protein, 224 aa, synthetic construct
  REGION 1..224: note = source = /note="Description of Artificial Sequence: Syntheticpolypeptide"
EVQLVQSGAEVKKPGESLKISCKGSGYAFTNYLIEWVRQMPGKGLEWMGVINPGSGGTNYNEKFKGQVTISADKSISTAYLQWSSLKASDTAMYYCARWRGDGYYAYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 618 - protein, 11 aa, synthetic construct
  REGION 1..11: note = source = /note="Description of Artificial Sequence: Syntheticpeptide"
RASQSVSSYLA

SEQ ID NO: 619 - protein, 7 aa, synthetic construct
  REGION 1..7: note = source = /note="Description of Artificial Sequence: Syntheticpeptide"
DASNRAT

SEQ ID NO: 620 - protein, 11 aa, synthetic construct
  REGION 1..11: note = source = /note="Description of Artificial Sequence: Syntheticpeptide"
QQRSNWPPALT

SEQ ID NO: 621 - protein, 7 aa, synthetic construct
  REGION 1..7: note = source = /note="Description of Artificial Sequence: Syntheticpeptide"
SQSVSSY

SEQ ID NO: 622 - skipped

SEQ ID NO: 623 - protein, 8 aa, synthetic construct
  REGION 1..8: note = source = /note="Description of Artificial Sequence: Syntheticpeptide"
RSNWPPAL

SEQ ID NO: 624 - protein, 6 aa, synthetic construct
  REGION 1..6: note = source = /note="Description of Artificial Sequence: Syntheticpeptide"
QSVSSY

SEQ ID NO: 625 - protein, 109 aa, synthetic construct
  REGION 1..109: note = source = /note="Description of Artificial Sequence: Syntheticpolypeptide"
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFCGGTKVEIK

SEQ ID NO: 626 - protein, 10 aa, synthetic construct
  REGION 1..10: note = source = /note="Description of Artificial Sequence: Syntheticpeptide"
GGSFSGYYWS

SEQ ID NO: 627 - protein, 16 aa, synthetic construct
  REGION 1..16: note = source = /note="Description of Artificial Sequence: Syntheticpeptide"
EINHGGYVTYNPSLES

SEQ ID NO: 628 - protein, 13 aa, synthetic construct
  REGION 1..13: note = source = /note="Description of Artificial Sequence: Syntheticpeptide"
DYGPGNYDWYFDL

SEQ ID NO: 629 - protein, 5 aa, synthetic construct
  REGION 1..5: note = source = /note="Description of Artificial Sequence: Syntheticpeptide"
GYYWS

SEQ ID NO: 630 - protein, 7 aa, synthetic construct
  REGION 1..7: note = source = /note="Description of Artificial Sequence: Syntheticpeptide"
GGSFSGY

SEQ ID NO: 631 - protein, 5 aa, synthetic construct
  REGION 1..5: note = source = /note="Description of Artificial Sequence: Syntheticpeptide"
NHGGY

SEQ ID NO: 632 - protein, 8 aa, synthetic construct
  REGION 1..8: note = source = /note="Description of Artificial Sequence: Syntheticpeptide"
GGSFSGYY

SEQ ID NO: 633 - protein, 7 aa, synthetic construct
  REGION 1..7: note = source = /note="Description of Artificial Sequence: Syntheticpeptide"
INHGGYV

SEQ ID NO: 634 - protein, 15 aa, synthetic construct
  REGION 1..15: note = source = /note="Description of Artificial Sequence: Syntheticpeptide"
ARDYGPGNYDWYFDL

SEQ ID NO: 635 - protein, 121 aa, synthetic construct [sequence omitted]

SEQ ID NO: 636 - protein, 11 aa, synthetic construct
  REGION 1..11: note = source = /note="Description of Artificial Sequence: Syntheticpeptide"
RASQDISSYLN

SEQ ID NO: 637 - protein, 7 aa, synthetic construct
  REGION 1..7: note = source = /note="Description of Artificial Sequence: Syntheticpeptide"
YTSRLHS

SEQ ID NO: 638 - protein, 7 aa, synthetic construct
  REGION 1..7: note = source = /note="Description of Artificial Sequence: Syntheticpeptide"
SQDISSY

SEQ ID NO: 639 - skipped

SEQ ID NO: 640 - protein, 6 aa, synthetic construct
  REGION 1..6: note = source = /note="Description of Artificial Sequence: Syntheticpeptide"
GNTLPY

SEQ ID NO: 641 - protein, 6 aa, synthetic construct
  REGION 1..6: note = source = /note="Description of Artificial Sequence: Syntheticpeptide"
QDISSY

SEQ ID NO: 642 - protein, 107 aa, synthetic construct [sequence omitted]

SEQ ID NO: 643 - protein, 11 aa, synthetic construct
  REGION 1..11: note = source = /note="Description of Artificial Sequence: Syntheticpeptide"
GYSITSDHAWS

SEQ ID NO: 644 - protein, 16 aa, synthetic construct
  REGION 1..16: note = source = /note="Description of Artificial Sequence: Syntheticpeptide"
YISYSGITTYNPSLKS

SEQ ID NO: 645 - protein, 10 aa, synthetic construct
  REGION 1..10: note = source = /note="Description of Artificial Sequence: Syntheticpeptide"
SLARTTAMDY

SEQ ID NO: 646 - protein, 6 aa, synthetic construct
  REGION 1..6: note = source = /note="Description of Artificial Sequence: Syntheticpeptide"
SDHAWS

SEQ ID NO: 647 - protein, 8 aa, synthetic construct
  REGION 1..8: note = source = /note="Description of Artificial Sequence: Syntheticpeptide"
GYSITSDH

SEQ ID NO: 648 - protein, 5 aa, synthetic construct
  REGION 1..5: note = source = /note="Description of Artificial Sequence: Syntheticpeptide"
SYSGI

SEQ ID NO: 649 - protein, 9 aa, synthetic construct
  REGION 1..9: note = source = /note="Description of Artificial Sequence: Syntheticpeptide"
GYSITSDHA

SEQ ID NO: 650 - protein, 7 aa, synthetic construct
  REGION 1..7: note = source = /note="Description of Artificial Sequence: Syntheticpeptide"
ISYSGIT

SEQ ID NO: 651 - protein, 12 aa, synthetic construct
  REGION 1..12: note = source = /note="Description of Artificial Sequence: Syntheticpeptide"
ARSLARTTAMDY

SEQ ID NO: 652 - protein, 119 aa, synthetic construct [sequence omitted]

SEQ ID NO: 653 - protein, 10 aa, synthetic construct
  REGION 1..10: note = source = /note="Description of Artificial Sequence: Syntheticpeptide"
QQSYSTPPIT

SEQ ID NO: 654 - protein, 7 aa, synthetic construct
  REGION 1..7: note = source = /note="Description of Artificial Sequence: Syntheticpeptide"
SYSTPPI

SEQ ID NO: 655 - protein, 108 aa, synthetic construct
  REGION 1..108: note = source = /note="Description of Artificial Sequence: Syntheticpolypeptide"
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 656 - protein, 10 aa, synthetic construct
  REGION 1..10: note = source = /note="Description of Artificial Sequence: Syntheticpeptide"
GFTFSDYYMT

SEQ ID NO: 657 - protein, 17 aa, synthetic construct
  REGION 1..17: note = source = /note="Description of Artificial Sequence: Syntheticpeptide"
YISSSGTNKYNADSVKG

SEQ ID NO: 658 - protein, 8 aa, synthetic construct
  REGION 1..8: note = source = /note="Description of Artificial Sequence: Syntheticpeptide"
DPPWGMDV

SEQ ID NO: 659 - protein, 5 aa, synthetic construct
  REGION 1..5: note = source = /note="Description of Artificial Sequence: Syntheticpeptide"
DYYMT

SEQ ID NO: 660 - protein, 6 aa, synthetic construct
  REGION 1..6: note = source = /note="Description of Artificial Sequence: Syntheticpeptide"
SSSGTN

SEQ ID NO: 661 - protein, 8 aa, synthetic construct
  REGION 1..8: note = source = /note="Description of Artificial Sequence: Syntheticpeptide"
GFTFSDYY

SEQ ID NO: 662 - protein, 8 aa, synthetic construct
  REGION 1..8: note = source = /note="Description of Artificial Sequence: Syntheticpeptide"
ISSSGTNK

SEQ ID NO: 663 - protein, 10 aa, synthetic construct
  REGION 1..10: note = source = /note="Description of Artificial Sequence: Syntheticpeptide"
VRDPPWGMDV

SEQ ID NO: 664 - protein, 117 aa, synthetic construct
  REGION 1..117: note = source = /note="Description of Artificial Sequence: Syntheticpolypeptide"
QVQLVESGGGLVKPGGSLRLSCAASGFTFSDYYMTWIRQTPGKGLDWVSYISSSGTNKYNADSVKGRFTISRDNAKNSLYLQMNSLRAEDTAVYYCVRDPPWGMDVWGQGTTVTVSS

SEQ ID NO: 665 - protein, 16 aa, synthetic construct
  REGION 1..16: note = source = /note="Description of Artificial Sequence: Syntheticpeptide"
RSSQSLLHSSGNTYLN

SEQ ID NO: 666 - protein, 7 aa, synthetic construct
  REGION 1..7: note = source = /note="Description of Artificial Sequence: Syntheticpeptide"
LVSKLES

SEQ ID NO: 667 - protein, 9 aa, synthetic construct
  REGION 1..9: note = source = /note="Description of Artificial Sequence: Syntheticpeptide"
MQFTHYPYT

SEQ ID NO: 668 - protein, 12 aa, synthetic construct
  REGION 1..12: note = source = /note="Description of Artificial Sequence: Syntheticpeptide"
SQSLLHSSGNTY

SEQ ID NO: 669 - skipped

SEQ ID NO: 670 - protein, 6 aa, synthetic construct
  REGION 1..6: note = source = /note="Description of Artificial Sequence: Syntheticpeptide"
FTHYPY

SEQ ID NO: 671 - protein, 11 aa, synthetic construct
  REGION 1..11: note = source = /note="Description of Artificial Sequence: Syntheticpeptide"
QSLLHSSGNTY

SEQ ID NO: 672 - protein, 112 aa, synthetic construct
  REGION 1..112: note = source = /note="Description of Artificial Sequence: Syntheticpolypeptide"
DVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIK

SEQ ID NO: 673 - protein, 219 aa, synthetic construct
  REGION 1..219: note = source = /note="Description of Artificial Sequence: Syntheticpolypeptide"
DVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 674 - protein, 10 aa, synthetic construct
  REGION 1..10: note = source = /note="Description of Artificial Sequence: Syntheticpeptide"
GYIFTEYYMY

SEQ ID NO: 675 - protein, 17 aa, synthetic construct
  REGION 1..17: note = source = /note="Description of Artificial Sequence: Syntheticpeptide"
RIDPEDGSIDYVEKFKK

SEQ ID NO: 676 - protein, 9 aa, synthetic construct
  REGION 1..9: note = source = /note="Description of Artificial Sequence: Syntheticpeptide"
GKFNYRFAY

SEQ ID NO: 677 - protein, 5 aa, synthetic construct
  REGION 1..5: note = source = /note="Description of Artificial Sequence: Syntheticpeptide"
EYYMY

SEQ ID NO: 678 - protein, 7 aa, synthetic construct
  REGION 1..7: note = source = /note="Description of Artificial Sequence: Syntheticpeptide"
GYIFTEY

SEQ ID NO: 679 - protein, 6 aa, synthetic construct
  REGION 1..6: note = source = /note="Description of Artificial Sequence: Syntheticpeptide"
DPEDGS

SEQ ID NO: 680 - protein, 8 aa, synthetic construct
  REGION 1..8: note = source = /note="Description of Artificial Sequence: Syntheticpeptide"
GYIFTEYY

SEQ ID NO: 681 - protein, 8 aa, synthetic construct
  REGION 1..8: note = source = /note="Description of Artificial Sequence: Syntheticpeptide"
IDPEDGSI

SEQ ID NO: 682 - protein, 11 aa, synthetic construct
  REGION 1..11: note = source = /note="Description of Artificial Sequence: Syntheticpeptide"
ARGKFNYRFAY

SEQ ID NO: 683 - protein, 118 aa, synthetic construct
  REGION 1..118: note = source = /note="Description of Artificial Sequence: Syntheticpolypeptide"
QVQLVQSGAEVQRPGASVKVSCKASGYIFTEYYMYWVRQAPGQGLELVGRIDPEDGSIDYVEKFKKKVTLTADTSSSTAYMELSSLTSDDTAVYYCARGKFNYRFAYWGQGTLVTVSS

SEQ ID NO: 684 - protein, 221 aa, synthetic construct
  REGION 1..221: note = source = /note="Description of Artificial Sequence: Syntheticpolypeptide"
QVQLVQSGAEVQRPGASVKVSCKASGYIFTEYYMYWVRQAPGQGLELVGRIDPEDGSIDYVEKFKKKVTLTADTSSSTAYMELSSLTSDDTAVYYCARGKFNYRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 685-700 - skipped (16 entries)

SEQ ID NO: 701 - protein, 227 aa, synthetic construct
  REGION 1..227: note = source = /note="Description of Artificial Sequence: Syntheticpolypeptide"
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 702 - protein, 227 aa, synthetic construct
  REGION 1..227: note = source = /note="Description of Artificial Sequence: Syntheticpolypeptide"
DKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 703 - protein, 711 aa, synthetic construct
  REGION 1..711: note = source = /note="Description of Artificial Sequence: Syntheticpolypeptide"
QVQLVQSGAEVQRPGASVKVSCKASGYIFTEYYMYWVRQAPGQGLELVGRIDPEDGSIDYVEKFKKKVTLTADTSSSTAYMELSSLTSDDTAVYYCARGKFNYRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQIT

SEQ ID NO: 704 - protein, 713 aa, synthetic construct
  REGION 1..713: note = source = /note="Description of Artificial Sequence: Syntheticpolypeptide"
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQIT

SEQ ID NO: 705 - protein, 226 aa, synthetic construct
  REGION 1..226: note = source = /note="Description of Artificial Sequence: Syntheticpolypeptide"
DIVLTQPPSVSGAPGQRVTISCSGSSSNIVSNYVNWYQQLPGTAPKLLIYDNNKRPSGVPDRFSGSKSGTSASLAITGLQSEDEADYYCQSYAIGSYSVVFGGGTKLTVLGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 706 - protein, 713 aa, synthetic construct
  REGION 1..713: note = source = /note="Description of Artificial Sequence: Syntheticpolypeptide"
QVQLVESGGGLVQPGGSLRLSCAASGFTFSTYGMSWVRQAPGKGLEWVSSIFYTGSSTYYADSVKGRFTISRDNSKNTLYLQMNSLRAEDTAVYYCARIGYAGDSKYAIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQIT

SEQ ID NO: 707 - protein, 720 aa, synthetic construct
  REGION 1..720: note = source = /note="Description of Artificial Sequence: Syntheticpolypeptide"
QVQLVQSGAEVQRPGASVKVSCKASGYIFTEYYMYWVRQAPGQGLELVGRIDPEDGSIDYVEKFKKKVTLTADTSSSTAYMELSSLTSDDTAVYYCARGKFNYRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSEVQLVESGGGLVQPGGSLKLSCAASGFTFNKYAMNWVRQAPGKCLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGCGTKLTVL

SEQ ID NO: 708 - protein, 715 aa, synthetic construct
  REGION 1..715: note = source = /note="Description of Artificial Sequence: Syntheticpolypeptide"
QVQLVQSGAEVQRPGASVKVSCKASGYIFTEYYMYWVRQAPGQGLELVGRIDPEDGSIDYVEKFKKKVTLTADTSSSTAYMELSSLTSDDTAVYYCARGKFNYRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSEVQLVESGGGLVQPGGSLKLSCAASGFTFNTYAMNWVRQASGKCLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGCGTKLTVL

SEQ ID NO: 709 - protein, 722 aa, synthetic construct
  REGION 1..722: note = source = /note="Description of Artificial Sequence: Syntheticpolypeptide"
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSEVQLVESGGGLVQPGGSLKLSCAASGFTFNKYAMNWVRQAPGKCLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGCGTKLTVL

SEQ ID NO: 710 - protein, 717 aa, synthetic construct
  REGION 1..717: note = source = /note="Description of Artificial Sequence: Syntheticpolypeptide"
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSEVQLVESGGGLVQPGGSLKLSCAASGFTFNTYAMNWVRQASGKCLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGCGTKLTVL

SEQ ID NO: 711 - protein, 713 aa, synthetic construct
  REGION 1..713: note = source = /note="Description of Artificial Sequence: Syntheticpolypeptide"
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITGGGGSGGGGSGGGGSGGGGSQVQLVQSGAEVQRPGASVKVSCKASGYIFTEYYMYWVRQAPGQGLELVGRIDPEDGSIDYVEKFKKKVTLTADTSSSTAYMELSSLTSDDTAVYYCARGKFNYRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 712 - protein, 715 aa, synthetic construct
  REGION 1..715: note = source = /note="Description of Artificial Sequence: Syntheticpolypeptide"
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITGGGGSGGGGSGGGGSGGGGSQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 713 - protein, 711 aa, synthetic construct
  REGION 1..711: note = source = /note="Description of Artificial Sequence: Syntheticpolypeptide"
QVQLVQSGAEVQRPGASVKVSCKASGYIFTEYYMYWVRQAPGQGLELVGRIDPEDGSIDYVEKFKKKVTLTADTSSSTAYMELSSLTSDDTAVYYCARGKFNYRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQIT

SEQ ID NO: 714 - protein, 713 aa, synthetic construct
  REGION 1..713: note = source = /note="Description of Artificial Sequence: Syntheticpolypeptide"
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQIT

SEQ ID NO: 715 - protein, 723 aa, synthetic construct
  REGION 1..723: note = source = /note="Description of Artificial Sequence: Syntheticpolypeptide"
QVQLVQSGAEVQRPGASVKVSCKASGYIFTEYYMYWVRQAPGQGLELVGRIDPEDGSIDYVEKFKKKVTLTADTSSSTAYMELSSLTSDDTAVYYCARGKFNYRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 716 - protein, 725 aa, synthetic construct
  REGION 1..725: note = source = /note="Description of Artificial Sequence: Syntheticpolypeptide"
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 717 - protein, 716 aa, synthetic construct
  REGION 1..716: note = source = /note="Description of Artificial Sequence: Syntheticpolypeptide"
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSQVQLVQSGAEVQRPGASVKVSCKASGYIFTEYYMYWVRQAPGQGLELVGRIDPEDGSIDYVEKFKKKVTLTADTSSSTAYMELSSLTSDDTAVYYCARGKFNYRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 718 - protein, 718 aa, synthetic construct
  REGION 1..718: note = source = /note="Description of Artificial Sequence: Syntheticpolypeptide"
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 719 - protein, 448 aa, synthetic construct
  REGION 1..448: note = source = /note="Description of Artificial Sequence: Syntheticpolypeptide"
QVQLVQSGAEVQRPGASVKVSCKASGYIFTEYYMYWVRQAPGQGLELVGRIDPEDGSIDYVEKFKKKVTLTADTSSSTAYMELSSLTSDDTAVYYCARGKFNYRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 720 - protein, 477 aa, synthetic construct
  REGION 1..477: note = source = /note="Description of Artificial Sequence: Syntheticpolypeptide"
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 721 - protein, 450 aa, synthetic construct
  REGION 1..450: note = source = /note="Description of Artificial Sequence: Syntheticpolypeptide"
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 722 - protein, 227 aa, synthetic construct
  REGION 1..227: note = source = /note="Description of Artificial Sequence: Syntheticpolypeptide"
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 723 - protein, 534 aa, synthetic construct
  REGION 1..534: note = source = /note="Description of Artificial Sequence: Syntheticpolypeptide"
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEEDPCACESLVKFQAKVEGLLQALTRKLEAVSKRLAILENTVV

SEQ ID NO: 724 - protein, 529 aa, synthetic construct
  REGION 1..529: note = source = /note="Description of Artificial Sequence: Syntheticpolypeptide"
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSDLGPQMLRELQETNAALQDVRELLRQQVREITFLKNTVMECDACGMQQ

SEQ ID NO: 725 - DNA, 2238 bp, synthetic construct
  misc_feature 1..2238: note = source = /note="Description of Artificial Sequence: Syntheticpolynucleotide"
  source 1..2238: mol_type = other DNA
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcttggcagaagccgccgcgaaagaagtgcagcttcaacaatcaggaccaggactcgtcaaaccatcacagaccctctccctcacatgtgccatctccggggactccatgttgagcaattccgacacttggaattggattagacaaagcccgtcccggggtctggaatggttgggacgcacctaccaccggtctacttggtacgacgactacgcgtcatccgtgcggggaagagtgtccatcaacgtggacacctccaagaaccagtacagcctgcagcttaatgccgtgactcctgaggatacgggcgtctactactgcgcccgcgtccgcctgcaagacgggaacagctggagcgatgcattcgatgtctggggccagggaactatggtcaccgtgtcgtctgggggcggtggatcgggtggcgggggttcggggggcggcggctctcagtccgctcttacccaaccggcctcagcctcggggagccccggccagagcgtgaccatttcctgcaccggcacttcatccgacgtgggcggctacaactacgtgtcctggtaccaacagcacccgggaaaggcccccaagctcatgatctacgacgtgtccaacaggccctcgggagtgtccaaccggttctcgggttcgaaatcgggaaacacagccagcctgaccatcagcggactgcaggctgaagatgaagccgactactactgctcctcctacacctcgtcatccacgctctacgtgttcggcactggaactcagctgactgtgctgaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 726 - protein, 746 aa, synthetic construct
  REGION 1..746: note = source = /note="Description of Artificial Sequence: Syntheticpolypeptide"
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSLAEAAAKEVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSSGGGGSGGGGSGGGGSQSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 727 - DNA, 2232 bp, synthetic construct
  misc_feature 1..2232: note = source = /note="Description of Artificial Sequence: Syntheticpolynucleotide"
  source 1..2232: mol_type = other DNA
atggccctccctgtcaccgccctgctgcttccgctggctcttctgctccacgccgctcggcccgaagtgcagcttcaacaatcaggaccaggactcgtcaaaccatcacagaccctctccctcacatgtgccatctccggggactccatgttgagcaattccgacacttggaattggattagacaaagcccgtcccggggtctggaatggttgggacgcacctaccaccggtctacttggtacgacgactacgcgtcatccgtgcggggaagagtgtccatcaacgtggacacctccaagaaccagtacagcctgcagcttaatgccgtgactcctgaggatacgggcgtctactactgcgcccgcgtccgcctgcaagacgggaacagctggagcgatgcattcgatgtctggggccagggaactatggtcaccgtgtcgtctgggggcggtggatcgggtggcgggggttcggggggcggcggctctcagtccgctcttacccaaccggcctcagcctcggggagccccggccagagcgtgaccatttcctgcaccggcacttcatccgacgtgggcggctacaactacgtgtcctggtaccaacagcacccgggaaaggcccccaagctcatgatctacgacgtgtccaacaggccctcgggagtgtccaaccggttctcgggttcgaaatcgggaaacacagccagcctgaccatcagcggactgcaggctgaagatgaagccgactactactgctcctcctacacctcgtcatccacgctctacgtgttcggcactggaactcagctgactgtgctgggagggggagggagtgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 728 - protein, 744 aa, synthetic construct
  REGION 1..744: note = source = /note="Description of Artificial Sequence: Syntheticpolypeptide"
MALPVTALLLPLALLLHAARPEVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSSGGGGSGGGGSGGGGSQSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVLGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 729 - DNA, 2202 bp, synthetic construct
  misc_feature 1..2202: note = source = /note="Description of Artificial Sequence: Syntheticpolynucleotide"
  source 1..2202: mol_type = other DNA
atggccctccctgtcaccgccctgctgcttccgctggctcttctgctccacgccgctcggccccagtccgctcttacccaaccggcctcagcctcggggagccccggccagagcgtgaccatttcctgcaccggcacttcatccgacgtgggcggctacaactacgtgtcctggtaccaacagcacccgggaaaggcccccaagctcatgatctacgacgtgtccaacaggccctcgggagtgtccaaccggttctcgggttcgaaatcgggaaacacagccagcctgaccatcagcggactgcaggctgaagatgaagccgactactactgctcctcctacacctcgtcatccacgctctacgtgttcggcactggaactcagctgactgtgctgggcggaggaggctccgaagtgcagcttcaacaatcaggaccaggactcgtcaaaccatcacagaccctctccctcacatgtgccatctccggggactccatgttgagcaattccgacacttggaattggattagacaaagcccgtcccggggtctggaatggttgggacgcacctaccaccggtctacttggtacgacgactacgcgtcatccgtgcggggaagagtgtccatcaacgtggacacctccaagaaccagtacagcctgcagcttaatgccgtgactcctgaggatacgggcgtctactactgcgcccgcgtccgcctgcaagacgggaacagctggagcgatgcattcgatgtctggggccagggaactatggtcaccgtgtcgtctggagggggagggagtgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 730 - protein, 734 aa, synthetic construct
  REGION 1..734: note = source = /note="Description of Artificial Sequence: Syntheticpolypeptide"
MALPVTALLLPLALLLHAARPQSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVLGGGGSEVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 731 - DNA, 2232 bp, synthetic construct
  misc_feature 1..2232: note = source = /note="Description of Artificial Sequence: Syntheticpolynucleotide"
  source 1..2232: mol_type = other DNA
atggccctgcccgtgactgcgctcctgcttccgttggccctgctcctgcatgccgccagacctcagtccgctctgactcagccggcctcagcttcggggtcccctggtcaaagcgtcactatttcctgtaccggaacctcatcagacgtgggcggctacaattacgtgtcctggtaccaacagcaccccggaaaggctcctaagcttatgatctacgacgtgtccaaccggccgtcaggagtgtccaacagattctccggctccaagagcggaaacactgccagcttgaccattagcggcttgcaggccgaggacgaagccgactactactgctctagctacacatcctcgtctaccctctacgtgtttggaacggggacccagctgactgtgctcgggggtggaggatcagaggtgcaactccagcagtccggtcctggcctcgtgaaaccgtcccaaaccctgtccctgacttgcgccatctcgggcgactccatgctgtccaattccgacacctggaactggattagacaatcgcctagccggggactcgaatggctgggccggacctaccaccggtccacgtggtatgacgactacgcaagctccgtccggggaagggtgtccattaacgtcgatacctccaagaaccagtacagccttcagctgaacgctgtgacccccgaggataccggcgtctactactgtgcaagagtgcgattgcaggatggaaactcgtggtcggacgcattcgatgtctggggacagggaactatggtgaccgtgtcctcgggcggaggcgggagcggaggaggaggctctggcggaggaggaagcgagattgtcatgactcagtccccggccacactctccctgtcacccggagaaagagcaaccctgagctgcagggcgtcccaggacatctcgaagtacctgaactggtaccagcagaagcctggacaagcaccccgcctcctgatctaccacacctcgcggctgcattcgggaatccccgccagattctcagggagcggatcaggaaccgactacaccctgactatctcgagcctgcaaccagaggatttcgccgtgtacttctgccagcaaggaaacaccctgccctacacctttggacagggaaccaagctcgagattaaggggggtggtggatcgggagggggtggatcaggaggaggcggctcacaagtccagctgcaagaatccggtccgggacttgtgaagccgtccgaaaccctgtcactgacttgcactgtgtccggggtgtcattgcccgactacggcgtgagctggattcggcagccccctggaaagggattggaatggatcggcgtgatctggggttcggaaactacctactatcagtcctcactgaagtcccgcgtgaccatcagcaaggataattccaaaaaccaagtgtctctgaagctctccagcgtcactgccgccgatactgccgtgtactactgcgccaagcactactattacggcggttcgtacgccatggactactggggccaagggacactcgtgaccgtgtcatccaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 732 - protein, 744 aa, synthetic construct
  REGION 1..744: note = source = /note="Description of Artificial Sequence: Syntheticpolypeptide"
MALPVTALLLPLALLLHAARPQSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVLGGGGSEVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 733 - DNA, 2232 bp, synthetic construct
  misc_feature 1..2232: note = source = /note="Description of Artificial Sequence: Syntheticpolynucleotide"
  source 1..2232: mol_type = other DNA
atggccctgcccgtgactgcgctcctgcttccgttggccctgctcctgcatgccgccagacctcagtccgctctgactcagccggcctcagcttcggggtcccctggtcaaagcgtcactatttcctgtaccggaacctcatcagacgtgggcggctacaattacgtgtcctggtaccaacagcaccccggaaaggctcctaagcttatgatctacgacgtgtccaaccggccgtcaggagtgtccaacagattctccggctccaagagcggaaacactgccagcttgaccattagcggcttgcaggccgaggacgaagccgactactactgctctagctacacatcctcgtctaccctctacgtgtttggaacggggacccagctgactgtgctcgggggtggaggatcagaggtgcaactccagcagtccggtcctggcctcgtgaaaccgtcccaaaccctgtccctgacttgcgccatctcgggcgactccatgctgtccaattccgacacctggaactggattagacaatcgcctagccggggactcgaatggctgggccggacctaccaccggtccacgtggtatgacgactacgcaagctccgtccggggaagggtgtccattaacgtcgatacctccaagaaccagtacagccttcagctgaacgctgtgacccccgaggataccggcgtctactactgtgcaagagtgcgattgcaggatggaaactcgtggtcggacgcattcgatgtctggggacagggaactatggtcactgtgtcctccggcggtggaggctcgggggggggcggctcaggaggaggcggctcacaagtccagctgcaagaatccggtccgggacttgtgaagccgtccgaaaccctgtcactgacttgcactgtgtccggggtgtcattgcccgactacggcgtgagctggattcggcagccccctggaaagggattggaatggatcggcgtgatctggggttcggaaactacctactatcagtcctcactgaagtcccgcgtgaccatcagcaaggataattccaaaaaccaagtgtctctgaagctctccagcgtcactgccgccgatactgccgtgtactactgcgccaagcactactattacggcggttcgtacgccatggactactggggacaaggcactcttgtgactgtgtcaagcggcggtggagggagcggtgggggcggttcaggaggaggcggatcagagatcgtgatgacccaatccccagccaccctgtccctcagccctggagaaagagccaccctgagctgccgggcctcccaggatatcagcaagtacttgaactggtaccaacaaaagccggggcaggcgccccggctcctgatctaccacacctcgcgcctccactcaggtatccccgccagattctcagggagcggctccggtactgactacaccctgactatttcctcactgcagccagaggactttgccgtgtacttctgccagcagggaaacactctgccgtacaccttcgggcagggaacgaagcttgaaattaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 734 - protein, 744 aa, synthetic construct
  REGION 1..744: note = source = /note="Description of Artificial Sequence: Syntheticpolypeptide"
MALPVTALLLPLALLLHAARPQSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVLGGGGSEVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 735 - DNA, 2985 bp, synthetic construct
  misc_feature 1..2985: note = source = /note="Description of Artificial Sequence: Syntheticpolynucleotide"
  source 1..2985: mol_type = other DNA
atggccctccctgtcaccgccctgctgcttccgctggctcttctgctccacgccgctcggcccgaagtgcagctgcagcagtcagggcctggcctggtcaagccgtcgcagaccctctccctgacatgcgccattagcggggactccatgctgagcaactcggacacctggaactggattcggcagtccccttcccggggactcgagtggctcggacgcacctaccatcggagcacttggtacgacgactacgcctcctccgtgagaggtcgcgtgtcgatcaacgtggatacctcgaagaaccagtatagcttgcaactgaacgccgtgacccctgaggataccggagtgtactattgtgcgagagtcaggctgcaagacggaaactcctggtccgacgcatttgatgtctggggacagggtactatggtcacggtgtcatctggaggcggaggatcgcaaagcgccctgactcagccggcttcggctagcggttcaccggggcagtccgtgactatctcctgcaccgggacttcctccgacgtgggaggctacaattacgtgtcctggtaccagcaacaccccggcaaagccccaaagctgatgatctacgacgtcagcaacagacccagcggagtgtccaaccggttcagcggctccaagtccggcaacaccgcctccctgaccatcagcgggcttcaggccgaagatgaggcggattactactgctcctcgtacacctcaagctcaactctgtacgtgttcggcaccggtactcagctcaccgtgctgaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggggaagcggagctactaacttcagcctgctgaagcaggctggagacgtggaggagaaccctggacctatggccttaccagtgaccgccttgctcctgccgctggccttgctgctccacgccgccaggccggaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 736 - protein, 995 aa, synthetic construct
  REGION 1..995: note = source = /note="Description of Artificial Sequence: Syntheticpolypeptide"
MALPVTALLLPLALLLHAARPEVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSSGGGGSQSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 737 - protein, 508 aa, synthetic construct
  REGION 1..508: note = source = /note="Description of Artificial Sequence: Syntheticpolypeptide"
MALPVTALLLPLALLLHAARPEVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSSGGGGSQSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG

SEQ ID NO: 738 - protein, 487 aa, synthetic construct
  REGION 1..487: note = source = /note="Description of Artificial Sequence: Syntheticpolypeptide"
PMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 739 - DNA, 2985 bp, synthetic construct
  misc_feature 1..2985: note = source = /note="Description of Artificial Sequence: Syntheticpolynucleotide"
  source 1..2985: mol_type = other DNA
atggccttaccagtgaccgccttgctcctgccgctggccttgctgctccacgccgccaggccggaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagctactaacttcagcctgctgaagcaggctggagacgtggaggagaaccctggacctatggccctccctgtcaccgccctgctgcttccgctggctcttctgctccacgccgctcggcccgaagtgcagctgcagcagtcagggcctggcctggtcaagccgtcgcagaccctctccctgacatgcgccattagcggggactccatgctgagcaactcggacacctggaactggattcggcagtccccttcccggggactcgagtggctcggacgcacctaccatcggagcacttggtacgacgactacgcctcctccgtgagaggtcgcgtgtcgatcaacgtggatacctcgaagaaccagtatagcttgcaactgaacgccgtgacccctgaggataccggagtgtactattgtgcgagagtcaggctgcaagacggaaactcctggtccgacgcatttgatgtctggggacagggtactatggtcacggtgtcatctggaggcggaggatcgcaaagcgccctgactcagccggcttcggctagcggttcaccggggcagtccgtgactatctcctgcaccgggacttcctccgacgtgggaggctacaattacgtgtcctggtaccagcaacaccccggcaaagccccaaagctgatgatctacgacgtcagcaacagacccagcggagtgtccaaccggttcagcggctccaagtccggcaacaccgcctccctgaccatcagcgggcttcaggccgaagatgaggcggattactactgctcctcgtacacctcaagctcaactctgtacgtgttcggcaccggtactcagctcaccgtgctgaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 740 - protein, 995 aa, synthetic construct
  REGION 1..995: note = source = /note="Description of Artificial Sequence: Syntheticpolypeptide"
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPEVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSSGGGGSQSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 741 - protein, 507 aa, synthetic construct
  REGION 1..507: note = source = /note="Description of Artificial Sequence: Syntheticpolypeptide"
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG

SEQ ID NO: 742 - protein, 488 aa, synthetic construct
  REGION 1..488: note = source = /note="Description of Artificial Sequence: Syntheticpolypeptide"
PMALPVTALLLPLALLLHAARPEVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSSGGGGSQSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 743 - DNA, 2985 bp, synthetic construct
  misc_feature 1..2985: note = source = /note="Description of Artificial Sequence: Syntheticpolynucleotide"
  source 1..2985: mol_type = other DNA
atggcacttcccgtcaccgccctgctgctcccactcgccctccttctgcacgccgcccgccccgaagtgcagctgcagcagtcaggaccgggcctggtcaaaccttcgcagactctgtccctgacttgcgctataagcggggactccatgctgagcaattcggacacttggaactggattcgccaaagccccagccggggtctggaatggctgggaaggacctaccatcgctctacttggtacgacgactacgccagctccgtgcgaggacgcgtgtccatcaacgtggacacctccaagaaccagtactcgcttcaactcaacgcagtgacccctgaagataccggagtctactattgcgcccgcgtgcggctccaggacgggaactcctggtcggacgctttcgatgtctggggacagggcactatggtcaccgtcagctccggcggcggcggtagccaatcggcgctgacacagccggcttccgcctcgggatcgcctggacagtcggtgaccatctcgtgcactggaacctcctccgacgtgggcggctacaattatgtgtcatggtaccagcagcacccgggaaaggcccctaagctgatgatctacgacgtgtccaatagacctagcggggtgtcaaacagattctccggatccaaatccggaaacactgcctccctgaccatttccggactgcaggccgaggacgaagccgattactactgctcctcttacacctcctcatccaccctctacgtgtttgggactgggacccagctgaccgtcctcactaccaccccggccccgcggccccctacaccggcaccgactattgccagccagcctctctcgctgcggccggaggcctgccgcccagccgccggcggagccgtgcacacccgcggtctggacttcgcgtgcgatatctacatctgggctccgctggccgggacttgtggcgtgctgctgctgtctctggtcatcacactgtactgcaagcgcggaagaaagaagctgctctacatcttcaagcaacccttcatgcggcctgtgcagaccacccaggaagaggatggctgctcctgccggttcccggaggaagaagagggcggatgcgaactgcgcgtgaagttcagccgaagcgccgacgccccggcctaccagcagggccagaaccaactgtacaacgaactcaacctgggtcggagagaagagtacgacgtgctggacaaaagacgcggcagggaccccgagatgggcggaaagcctcgccgcaagaacccgcaggagggcctctacaacgagctgcagaaggacaagatggccgaagcctactcagagatcggcatgaagggggagcggaggcgcgggaagggccacgacggtttgtaccaaggactttccactgcgaccaaggacacctacgatgccctccatatgcaagccctgccgccccggggttccggagctaccaacttctcgctgttgaagcaggccggagatgtcgaggaaaacccgggacctatggccctgccagtgaccgcgctcctgctgcccctggctctgctgcttcacgcggcccggcctgagattgtgatgactcagagcccggcgaccctgtccctgtcccccggggagagagcaaccctgtcgtgccgggcctcccaagacatctcaaagtacctcaattggtatcagcagaagccaggacaggctccacggttgctgatctaccacacttcgagactgcactcaggaatccccgcgcggttttccggttccggctccgggaccgactacaccctgaccatcagctcgctccagcctgaggatttcgcagtgtacttctgtcagcaaggaaacacccttccatacaccttcggacagggtaccaagctggaaatcaagggaggaggaggatctgggggcggtggttccggaggcggtggaagccaagtgcagctccaggaaagcggacccgggctggtcaagccgagcgaaaccctctcactgacttgtactgtgtccggagtgtccctgcctgactatggagtgtcctggatccgacagccccccggaaagggtctggagtggattggggtcatctggggctccgaaactacctactaccagagcagcctcaagagccgggtcaccatttcaaaggataactccaagaatcaagtgtccctgaagctgtcctcagtgacagccgcagacaccgccgtgtactactgcgccaagcactactactacggaggctcctacgcaatggactactggggacaaggcactttggtcactgtgtcaagcaccaccacccctgcgcctcggcctcctaccccggctcccactatcgcgagccagccgctgagcctgcggcctgaggcttgccgaccggccgctggcggcgccgtgcatactcggggcctcgactttgcctgtgacatctacatctgggcccccctggccggaacgtgcggagtgctgctgctgtcgctggtcattaccctgtattgcaaacgcggaaggaagaagctgttgtacattttcaagcagcccttcatgcgcccggtgcaaactactcaggaggaagatggctgttcctgtcggttccccgaagaggaagaaggcggctgcgagttgagggtcaagttctcccggtccgccgatgctcccgcctaccaacaggggcagaaccagctttataacgaactgaacctgggcaggagggaggaatatgatgtgttggataagcgccggggccgggacccagaaatggggggaaagcccagaagaaagaaccctcaagagggactttacaacgaattgcagaaagacaaaatggccgaggcctactccgagattgggatgaagggcgaaagacggagaggaaaggggcacgacgggctctaccagggactcagcaccgccaccaaagatacctacgacgccctgcatatgcaggcgctgccgccgcgc

SEQ ID NO: 744 - protein, 9 aa, synthetic construct
  REGION 1..9: note = source = /note="Description of Artificial Sequence: Syntheticpeptide"
GDSMLSNSD

SEQ ID NO: 745 - protein, 7 aa, synthetic construct
  REGION 1..7: note = source = /note="Description of Artificial Sequence: Syntheticpeptide"
YHRSTWY

SEQ ID NO: 746 - protein, 10 aa, synthetic construct
  REGION 1..10: note = source = /note="Description of Artificial Sequence: Syntheticpeptide"
GDSMLSNSDT

SEQ ID NO: 747 - protein, 9 aa, synthetic construct
  REGION 1..9: note = source = /note="Description of Artificial Sequence: Syntheticpeptide"
TYHRSTWYD

SEQ ID NO: 748 - protein, 17 aa, synthetic construct
  REGION 1..17: note = source = /note="Description of Artificial Sequence: Syntheticpeptide"
ARVRLQDGNSWSDAFDV

SEQ ID NO: 749 - protein, 10 aa, synthetic construct
  REGION 1..10: note = source = /note="Description of Artificial Sequence: Syntheticpeptide"
GVSLPDYGVS

SEQ ID NO: 750 - protein, 16 aa, synthetic construct
  REGION 1..16: note = source = /note="Description of Artificial Sequence: Syntheticpeptide"
VIWGSETTYYSSSLKS

SEQ ID NO: 751 - protein, 16 aa, synthetic construct
  REGION 1..16: note = source = /note="Description of Artificial Sequence: Syntheticpeptide"
VIWGSETTYYNSSLKS

SEQ ID NO: 752 - DNA, 726 bp, synthetic construct
  misc_feature 1..726: note = source = /note="Description of Artificial Sequence: Syntheticpolynucleotide"
  source 1..726: mol_type = other DNA
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagc

SEQ ID NO: 753 - DNA, 726 bp, synthetic construct
  misc_feature 1..726: note = source = /note="Description of Artificial Sequence: Syntheticpolynucleotide"
  source 1..726: mol_type = other DNA
gagattgtcatgactcagtccccggccacactctccctgtcacccggagaaagagcaaccctgagctgcagggcgtcccaggacatctcgaagtacctgaactggtaccagcagaagcctggacaagcaccccgcctcctgatctaccacacctcgcggctgcattcgggaatccccgccagattctcagggagcggatcaggaaccgactacaccctgactatctcgagcctgcaaccagaggatttcgccgtgtacttctgccagcaaggaaacaccctgccctacacctttggacagggaaccaagctcgagattaaggggggtggtggatcgggagggggtggatcaggaggaggcggctcacaagtccagctgcaagaatccggtccgggacttgtgaagccgtccgaaaccctgtcactgacttgcactgtgtccggggtgtcattgcccgactacggcgtgagctggattcggcagccccctggaaagggattggaatggatcggcgtgatctggggttcggaaactacctactatcagtcctcactgaagtcccgcgtgaccatcagcaaggataattccaaaaaccaagtgtctctgaagctctccagcgtcactgccgccgatactgccgtgtactactgcgccaagcactactattacggcggttcgtacgccatggactactggggccaagggacactcgtgaccgtgtcatcc

SEQ ID NO: 754 - DNA, 726 bp, synthetic construct
  misc_feature 1..726: note = source = /note="Description of Artificial Sequence: Syntheticpolynucleotide"
  source 1..726: mol_type = other DNA
caagtccagctgcaagaatccggtccgggacttgtgaagccgtccgaaaccctgtcactgacttgcactgtgtccggggtgtcattgcccgactacggcgtgagctggattcggcagccccctggaaagggattggaatggatcggcgtgatctggggttcggaaactacctactatcagtcctcactgaagtcccgcgtgaccatcagcaaggataattccaaaaaccaagtgtctctgaagctctccagcgtcactgccgccgatactgccgtgtactactgcgccaagcactactattacggcggttcgtacgccatggactactggggacaaggcactcttgtgactgtgtcaagcggcggtggagggagcggtgggggcggttcaggaggaggcggatcagagatcgtgatgacccaatccccagccaccctgtccctcagccctggagaaagagccaccctgagctgccgggcctcccaggatatcagcaagtacttgaactggtaccaacaaaagccggggcaggcgccccggctcctgatctaccacacctcgcgcctccactcaggtatccccgccagattctcagggagcggctccggtactgactacaccctgactatttcctcactgcagccagaggactttgccgtgtacttctgccagcagggaaacactctgccgtacaccttcgggcagggaacgaagcttgaaattaag

SEQ ID NO: 755 - protein, 242 aa, synthetic construct
  REGION 1..242: note = source = /note="Description of Artificial Sequence: Syntheticpolypeptide"
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 756 - DNA, 726 bp, synthetic construct
  misc_feature 1..726: note = source = /note="Description of Artificial Sequence: Syntheticpolynucleotide"
  source 1..726: mol_type = other DNA
gagattgtgatgactcagagcccggcgaccctgtccctgtcccccggggagagagcaaccctgtcgtgccgggcctcccaagacatctcaaagtacctcaattggtatcagcagaagccaggacaggctccacggttgctgatctaccacacttcgagactgcactcaggaatccccgcgcggttttccggttccggctccgggaccgactacaccctgaccatcagctcgctccagcctgaggatttcgcagtgtacttctgtcagcaaggaaacacccttccatacaccttcggacagggtaccaagctggaaatcaagggaggaggaggatctgggggcggtggttccggaggcggtggaagccaagtgcagctccaggaaagcggacccgggctggtcaagccgagcgaaaccctctcactgacttgtactgtgtccggagtgtccctgcctgactatggagtgtcctggatccgacagccccccggaaagggtctggagtggattggggtcatctggggctccgaaactacctactaccagagcagcctcaagagccgggtcaccatttcaaaggataactccaagaatcaagtgtccctgaagctgtcctcagtgacagccgcagacaccgccgtgtactactgcgccaagcactactactacggaggctcctacgcaatggactactggggacaaggcactttggtcactgtgtcaagc

SEQ ID NO: 757 - DNA, 729 bp, synthetic construct
  misc_feature 1..729: note = source = /note="Description of Artificial Sequence: Syntheticpolynucleotide"
  source 1..729: mol_type = other DNA
gaagtgcagctgcagcagtcagggcctggcctggtcaagccgtcgcagaccctctccctgacatgcgccattagcggggactccatgctgagcaactcggacacctggaactggattcggcagtccccttcccggggactcgagtggctcggacgcacctaccatcggagcacttggtacgacgactacgcctcctccgtgagaggtcgcgtgtcgatcaacgtggatacctcgaagaaccagtatagcttgcaactgaacgccgtgacccctgaggataccggagtgtactattgtgcgagagtcaggctgcaagacggaaactcctggtccgacgcatttgatgtctggggacagggtactatggtcacggtgtcatctggaggcggaggatcgcaaagcgccctgactcagccggcttcggctagcggttcaccggggcagtccgtgactatctcctgcaccgggacttcctccgacgtgggaggctacaattacgtgtcctggtaccagcaacaccccggcaaagccccaaagctgatgatctacgacgtcagcaacagacccagcggagtgtccaaccggttcagcggctccaagtccggcaacaccgcctccctgaccatcagcgggcttcaggccgaagatgaggcggattactactgctcctcgtacacctcaagctcaactctgtacgtgttcggcaccggtactcagctcaccgtgctg

SEQ ID NO: 758 - protein, 243 aa, synthetic construct
  REGION 1..243: note = source = /note="Description of Artificial Sequence: Syntheticpolypeptide"
EVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSSGGGGSQSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVL

SEQ ID NO: 759 - DNA, 729 bp, synthetic construct
  misc_feature 1..729: note = source = /note="Description of Artificial Sequence: Syntheticpolynucleotide"
  source 1..729: mol_type = other DNA
gaagtgcagctgcagcagtcaggaccgggcctggtcaaaccttcgcagactctgtccctgacttgcgctataagcggggactccatgctgagcaattcggacacttggaactggattcgccaaagccccagccggggtctggaatggctgggaaggacctaccatcgctctacttggtacgacgactacgccagctccgtgcgaggacgcgtgtccatcaacgtggacacctccaagaaccagtactcgcttcaactcaacgcagtgacccctgaagataccggagtctactattgcgcccgcgtgcggctccaggacgggaactcctggtcggacgctttcgatgtctggggacagggcactatggtcaccgtcagctccggcggcggcggtagccaatcggcgctgacacagccggcttccgcctcgggatcgcctggacagtcggtgaccatctcgtgcactggaacctcctccgacgtgggcggctacaattatgtgtcatggtaccagcagcacccgggaaaggcccctaagctgatgatctacgacgtgtccaatagacctagcggggtgtcaaacagattctccggatccaaatccggaaacactgcctccctgaccatttccggactgcaggccgaggacgaagccgattactactgctcctcttacacctcctcatccaccctctacgtgtttgggactgggacccagctgaccgtcctc

SEQ ID NO: 760 - DNA, 759 bp, synthetic construct
  misc_feature 1..759: note = source = /note="Description of Artificial Sequence: Syntheticpolynucleotide"
  source 1..759: mol_type = other DNA
gaagtgcagcttcaacaatcaggaccaggactcgtcaaaccatcacagaccctctccctcacatgtgccatctccggggactccatgttgagcaattccgacacttggaattggattagacaaagcccgtcccggggtctggaatggttgggacgcacctaccaccggtctacttggtacgacgactacgcgtcatccgtgcggggaagagtgtccatcaacgtggacacctccaagaaccagtacagcctgcagcttaatgccgtgactcctgaggatacgggcgtctactactgcgcccgcgtccgcctgcaagacgggaacagctggagcgatgcattcgatgtctggggccagggaactatggtcaccgtgtcgtctgggggcggtggatcgggtggcgggggttcggggggcggcggctctcagtccgctcttacccaaccggcctcagcctcggggagccccggccagagcgtgaccatttcctgcaccggcacttcatccgacgtgggcggctacaactacgtgtcctggtaccaacagcacccgggaaaggcccccaagctcatgatctacgacgtgtccaacaggccctcgggagtgtccaaccggttctcgggttcgaaatcgggaaacacagccagcctgaccatcagcggactgcaggctgaagatgaagccgactactactgctcctcctacacctcgtcatccacgctctacgtgttcggcactggaactcagctgactgtgctg

SEQ ID NO: 761 - DNA, 729 bp, synthetic construct
  misc_feature 1..729: note = source = /note="Description of Artificial Sequence: Syntheticpolynucleotide"
  source 1..729: mol_type = other DNA
cagtccgctcttacccaaccggcctcagcctcggggagccccggccagagcgtgaccatttcctgcaccggcacttcatccgacgtgggcggctacaactacgtgtcctggtaccaacagcacccgggaaaggcccccaagctcatgatctacgacgtgtccaacaggccctcgggagtgtccaaccggttctcgggttcgaaatcgggaaacacagccagcctgaccatcagcggactgcaggctgaagatgaagccgactactactgctcctcctacacctcgtcatccacgctctacgtgttcggcactggaactcagctgactgtgctgggcggaggaggctccgaagtgcagcttcaacaatcaggaccaggactcgtcaaaccatcacagaccctctccctcacatgtgccatctccggggactccatgttgagcaattccgacacttggaattggattagacaaagcccgtcccggggtctggaatggttgggacgcacctaccaccggtctacttggtacgacgactacgcgtcatccgtgcggggaagagtgtccatcaacgtggacacctccaagaaccagtacagcctgcagcttaatgccgtgactcctgaggatacgggcgtctactactgcgcccgcgtccgcctgcaagacgggaacagctggagcgatgcattcgatgtctggggccagggaactatggtcaccgtgtcgtct

SEQ ID NO: 762 - protein, 243 aa, synthetic construct
  REGION 1..243: note = source = /note="Description of Artificial Sequence: Syntheticpolypeptide"
QSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVLGGGGSEVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSS

SEQ ID NO: 763 - DNA, 729 bp, synthetic construct
  misc_feature 1..729: note = source = /note="Description of Artificial Sequence: Syntheticpolynucleotide"
  source 1..729: mol_type = other DNA
cagtccgctctgactcagccggcctcagcttcggggtcccctggtcaaagcgtcactatttcctgtaccggaacctcatcagacgtgggcggctacaattacgtgtcctggtaccaacagcaccccggaaaggctcctaagcttatgatctacgacgtgtccaaccggccgtcaggagtgtccaacagattctccggctccaagagcggaaacactgccagcttgaccattagcggcttgcaggccgaggacgaagccgactactactgctctagctacacatcctcgtctaccctctacgtgtttggaacggggacccagctgactgtgctcgggggtggaggatcagaggtgcaactccagcagtccggtcctggcctcgtgaaaccgtcccaaaccctgtccctgacttgcgccatctcgggcgactccatgctgtccaattccgacacctggaactggattagacaatcgcctagccggggactcgaatggctgggccggacctaccaccggtccacgtggtatgacgactacgcaagctccgtccggggaagggtgtccattaacgtcgatacctccaagaaccagtacagccttcagctgaacgctgtgacccccgaggataccggcgtctactactgtgcaagagtgcgattgcaggatggaaactcgtggtcggacgcattcgatgtctggggacagggaactatggtgaccgtgtcctcg

SEQ ID NO: 764 - DNA, 729 bp, synthetic construct
  misc_feature 1..729: note = source = /note="Description of Artificial Sequence: Syntheticpolynucleotide"
  source 1..729: mol_type = other DNA
cagtccgctctgactcagccggcctcagcttcggggtcccctggtcaaagcgtcactatttcctgtaccggaacctcatcagacgtgggcggctacaattacgtgtcctggtaccaacagcaccccggaaaggctcctaagcttatgatctacgacgtgtccaaccggccgtcaggagtgtccaacagattctccggctccaagagcggaaacactgccagcttgaccattagcggcttgcaggccgaggacgaagccgactactactgctctagctacacatcctcgtctaccctctacgtgtttggaacggggacccagctgactgtgctcgggggtggaggatcagaggtgcaactccagcagtccggtcctggcctcgtgaaaccgtcccaaaccctgtccctgacttgcgccatctcgggcgactccatgctgtccaattccgacacctggaactggattagacaatcgcctagccggggactcgaatggctgggccggacctaccaccggtccacgtggtatgacgactacgcaagctccgtccggggaagggtgtccattaacgtcgatacctccaagaaccagtacagccttcagctgaacgctgtgacccccgaggataccggcgtctactactgtgcaagagtgcgattgcaggatggaaactcgtggtcggacgcattcgatgtctggggacagggaactatggtcactgtgtcctcc

SEQ ID NO: 765 - DNA, 63 bp, synthetic construct
  misc_feature 1..63: note = source = /note="Description of Artificial Sequence: Syntheticoligonucleotide"
  source 1..63: mol_type = other DNA
atggccctgcccgtgactgcgctcctgcttccgttggccctgctcctgcatgccgccagacct

SEQ ID NO: 766 - DNA, 63 bp, synthetic construct
  misc_feature 1..63: note = source = /note="Description of Artificial Sequence: Syntheticoligonucleotide"
  source 1..63: mol_type = other DNA
atggccttaccagtgaccgccttgctcctgccgctggccttgctgctccacgccgccaggccg

SEQ ID NO: 767 - DNA, 63 bp, synthetic construct
  misc_feature 1..63: note = source = /note="Description of Artificial Sequence: Syntheticoligonucleotide"
  source 1..63: mol_type = other DNA
atggcacttcccgtcaccgccctgctgctcccactcgccctccttctgcacgccgcccgcccc

SEQ ID NO: 768 - DNA, 63 bp, synthetic construct
  misc_feature 1..63: note = source = /note="Description of Artificial Sequence: Syntheticoligonucleotide"
  source 1..63: mol_type = other DNA
atggccctgccagtgaccgcgctcctgctgcccctggctctgctgcttcacgcggcccggcct

SEQ ID NO: 769 - DNA, 207 bp, synthetic construct
  misc_feature 1..207: note = source = /note="Description of Artificial Sequence: Syntheticpolynucleotide"
  source 1..207: mol_type = other DNA
accactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgt

SEQ ID NO: 770 - DNA, 207 bp, synthetic construct
  misc_feature 1..207: note = source = /note="Description of Artificial Sequence: Syntheticpolynucleotide"
  source 1..207: mol_type = other DNA
actaccaccccggccccgcggccccctacaccggcaccgactattgccagccagcctctctcgctgcggccggaggcctgccgcccagccgccggcggagccgtgcacacccgcggtctggacttcgcgtgcgatatctacatctgggctccgctggccgggacttgtggcgtgctgctgctgtctctggtcatcacactgtactgc

SEQ ID NO: 771 - DNA, 207 bp, synthetic construct
  misc_feature 1..207: note = source = /note="Description of Artificial Sequence: Syntheticpolynucleotide"
  source 1..207: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgc

SEQ ID NO: 772 - DNA, 207 bp, synthetic construct
  misc_feature 1..207: note = source = /note="Description of Artificial Sequence: Syntheticpolynucleotide"
  source 1..207: mol_type = other DNA
accaccacccctgcgcctcggcctcctaccccggctcccactatcgcgagccagccgctgagcctgcggcctgaggcttgccgaccggccgctggcggcgccgtgcatactcggggcctcgactttgcctgtgacatctacatctgggcccccctggccggaacgtgcggagtgctgctgctgtcgctggtcattaccctgtattgc

SEQ ID NO: 773 - DNA, 126 bp, synthetic construct
  misc_feature 1..126: note = source = /note="Description of Artificial Sequence: Syntheticpolynucleotide"
  source 1..126: mol_type = other DNA
aagcgcggtcggaagaagctgctgtacatctttaagcaacccttcatgaggcctgtgcagactactcaagaggaggacggctgttcatgccggttcccagaggaggaggaaggcggctgcgaactg

SEQ ID NO: 774 - DNA, 126 bp, synthetic construct
  misc_feature 1..126: note = source = /note="Description of Artificial Sequence: Syntheticpolynucleotide"
  source 1..126: mol_type = other DNA
aagcgcggaagaaagaagctgctctacatcttcaagcaacccttcatgcggcctgtgcagaccacccaggaagaggatggctgctcctgccggttcccggaggaagaagagggcggatgcgaactg

SEQ ID NO: 775 - DNA, 126 bp, synthetic construct
  misc_feature 1..126: note = source = /note="Description of Artificial Sequence: Syntheticpolynucleotide"
  source 1..126: mol_type = other DNA
aaacgcggaaggaagaagctgttgtacattttcaagcagcccttcatgcgcccggtgcaaactactcaggaggaagatggctgttcctgtcggttccccgaagaggaagaaggcggctgcgagttg

SEQ ID NO: 776 - DNA, 336 bp, synthetic construct
  misc_feature 1..336: note = source = /note="Description of Artificial Sequence: Syntheticpolynucleotide"
  source 1..336: mol_type = other DNA
cgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 777 - DNA, 336 bp, synthetic construct
  misc_feature 1..336: note = source = /note="Description of Artificial Sequence: Syntheticpolynucleotide"
  source 1..336: mol_type = other DNA
cgcgtgaagttcagccgaagcgccgacgccccggcctaccagcagggccagaaccaactgtacaacgaactcaacctgggtcggagagaagagtacgacgtgctggacaaaagacgcggcagggaccccgagatgggcggaaagcctcgccgcaagaacccgcaggagggcctctacaacgagctgcagaaggacaagatggccgaagcctactcagagatcggcatgaagggggagcggaggcgcgggaagggccacgacggtttgtaccaaggactttccactgcgaccaaggacacctacgatgccctccatatgcaagccctgccgccccgg

SEQ ID NO: 778 - DNA, 336 bp, synthetic construct
  misc_feature 1..336: note = source = /note="Description of Artificial Sequence: Syntheticpolynucleotide"
  source 1..336: mol_type = other DNA
agggtcaagttctcccggtccgccgatgctcccgcctaccaacaggggcagaaccagctttataacgaactgaacctgggcaggagggaggaatatgatgtgttggataagcgccggggccgggacccagaaatggggggaaagcccagaagaaagaaccctcaagagggactttacaacgaattgcagaaagacaaaatggccgaggcctactccgagattgggatgaagggcgaaagacggagaggaaaggggcacgacgggctctaccagggactcagcaccgccaccaaagatacctacgacgccctgcatatgcaggcgctgccgccgcgc

SEQ ID NO: 779 - DNA, 21 bp, synthetic construct
  misc_feature 1..21: note = source = /note="Description of Artificial Sequence: Syntheticoligonucleotide"
  source 1..21: mol_type = other DNA
ttggcagaagccgccgcgaaa

SEQ ID NO: 780 - DNA, 45 bp, synthetic construct
  misc_feature 1..45: note = source = /note="Description of Artificial Sequence: Syntheticoligonucleotide"
  source 1..45: mol_type = other DNA
ggtggaggtggcagcggaggaggtgggtccggcggtggaggaagc

SEQ ID NO: 781 - DNA, 45 bp, synthetic construct
  misc_feature 1..45: note = source = /note="Description of Artificial Sequence: Syntheticoligonucleotide"
  source 1..45: mol_type = other DNA
ggcggaggcgggagcggaggaggaggctctggcggaggaggaagc

SEQ ID NO: 782 - DNA, 45 bp, synthetic construct
  misc_feature 1..45: note = source = /note="Description of Artificial Sequence: Syntheticoligonucleotide"
  source 1..45: mol_type = other DNA
ggcggtggaggctcgggggggggcggctcaggaggaggcggctca

SEQ ID NO: 783 - DNA, 66 bp, synthetic construct
  misc_feature 1..66: note = source = /note="Description of Artificial Sequence: Syntheticoligonucleotide"
  source 1..66: mol_type = other DNA
ggaagcggagctactaacttcagcctgctgaagcaggctggagacgtggaggagaaccctggacct

SEQ ID NO: 784 - protein, 22 aa, synthetic construct
  REGION 1..22: note = source = /note="Description of Artificial Sequence: Syntheticpeptide"
GSGATNFSLLKQAGDVEENPGP

SEQ ID NO: 785 - DNA, 66 bp, synthetic construct
  misc_feature 1..66: note = source = /note="Description of Artificial Sequence: Syntheticoligonucleotide"
  source 1..66: mol_type = other DNA
ggttccggagctaccaacttctcgctgttgaagcaggccggagatgtcgaggaaaacccgggacct

SEQ ID NO: 786 - DNA, 15 bp, synthetic construct
  misc_feature 1..15: note = source = /note="Description of Artificial Sequence: Syntheticoligonucleotide"
  source 1..15: mol_type = other DNA
ggtggaggtggcagc

SEQ ID NO: 787 - DNA, 29 bp, synthetic construct
  misc_feature 1..29: note = source = /note="Description of Artificial Sequence: Syntheticoligonucleotide"
  source 1..29: mol_type = other DNA
tatatatcttgtggaaaggacgaaacacc

SEQ ID NO: 788 - DNA, 30 bp, synthetic construct
  misc_feature 1..30: note = source = /note="Description of Artificial Sequence: Syntheticoligonucleotide"
  source 1..30: mol_type = other DNA
tatatatcttgtggaaaggacgaaacaccg

SEQ ID NO: 789 - DNA, 30 bp, synthetic construct
  misc_feature 1..30: note = source = /note="Description of Artificial Sequence: Syntheticoligonucleotide"
  source 1..30: mol_type = other DNA
ttatatatcttgtggaaaggacgaaacacc

SEQ ID NO: 790 - DNA, 31 bp, synthetic construct
  misc_feature 1..31: note = source = /note="Description of Artificial Sequence: Syntheticoligonucleotide"
  source 1..31: mol_type = other DNA
ttatatatcttgtggaaaggacgaaacacca

SEQ ID NO: 791 - DNA, 31 bp, synthetic construct
  misc_feature 1..31: note = source = /note="Description of Artificial Sequence: Syntheticoligonucleotide"
  source 1..31: mol_type = other DNA
ttatatatcttgtggaaaggacgaaacaccg

SEQ ID NO: 792 - DNA, 15 bp, synthetic construct
  misc_feature 1..15: note = source = /note="Description of Artificial Sequence: Syntheticoligonucleotide"
  source 1..15: mol_type = other DNA
gttaatattcatagc

SEQ ID NO: 793 - skipped